(19) United States
(12) Patent Application Publication
(10) Pub. No.: US 2020/0063182 A1
(43) Pub. Date: Feb. 27, 2020

(54) FLUOROPHORE-BASED KINASE SENSORS

(71) Applicant: AssayQuant Technologies, Inc., Marlborough, MA (US)

(72) Inventors: Erik M. Schaefer, Hopkinton, MA (US); Barbara Imperiali, Cambridge, MA (US)

(21) Appl. No.: 16/099,044

(22) PCT Filed: May 4, 2017
(86) PCT No.: PCT/US17/31059

§ 371 Date: Nov. 5, 2018

Related U.S. Application Data
(60) Provisional application No. 62/331,903, filed on May 4, 2016.

(51) Int. Cl. C12Q 1/48; C07K 7/06; C07D 215/36; C07D 215/26; C07D 221/08

(52) U.S. Cl. CPC C12Q 1/485; C07K 7/06; A61K 38/00; C07D 215/26; C07D 221/08; C07D 215/36

(57) ABSTRACT

Disclosed are metal-binding analytical reagents that exhibit chelation-enhanced fluorescence (CHEF) upon binding to Mg²⁺. Also disclosed are methods of using the reagents for detecting the presence of or quantifying the activity of individual kinases or groups of kinases in a sample.

### RELATED APPLICATION

This application claims the benefit of priority to U.S. Provisional Patent Application Ser. No. 62/331,903, filed May 4, 2016.

### SEQUENCE LISTING

The instant application contains a Sequence Listing which has been submitted electronically in ASCII format and is hereby incorporated by reference in its entirety. Said ASCII copy, created on Jul. 14, 2017, is named “AQT-Sequence-References.TXT” and is 3,451 bytes in size.

### BACKGROUND OF THE INVENTION

Protein kinases are involved in all aspects of regulation within cells. A protein kinase catalyzes the transfer of a phosphoryl group from adenosine triphosphate (ATP) to a serine, threonine or tyrosine residue in a peptide or protein sequence. Each kinase is specific for the amino acids surrounding the residue to be phosphorylated. The traditional method for assaying kinase activity is discontinuous and requires ³²P-labelled ATP, which requires special handling.

Many companies market specialized fluorescent kinase assay systems, all of which are discontinuous, requiring sampling of the reaction mixture or additional handling steps or use of antibodies to identify the product of the reaction or create the fluorescent signal (e.g., Promega. Thermo Fisher, Calbiochem. Cell Signaling Technology, Molecular Devices/Danaher, DiscoveRx. EMD-Millipore, PerkinElmer). A continuous fluorescent assay that can be performed in real time is of great utility. Currently, few examples of sensors capable of such assays exist. Approaches include: environment-sensitive fluorophores near the phosphorylation site (Wright, D. E. et al. Proc. Natl. Acad. Sci. USA 1981, 78, 6048-6050; Mcllroy, B. K. et al. Anal. Biochem. 1991, 195, 148-152; Higashi, H. et al. FEBS Lett. 1997, 414, 55-60; Post, P. L. et al. J. Biol. Chem. 1994, 269, 12880-12887), FRET pairs flanking a sequence which undergoes a conformational change upon phosphorylation (Nagai, Y. et al. Nat. Biotech. 2000, 18, 313-316; Ohuchi, Y. et al. Analyst 2000, 125, 1905-1907; Zhang, J. et al. Proc. Natl. Acad. Sci. USA 2001, 98, 14997-15002; Ting, A. Y. et al. Proc. Natl. Acad. Sci. USA 2001, 98, 15003-15008; Hofmann, R. M. et al. Bioorg. Med. Chem. Lett. 2001, 11, 3091-3094: Kurokawa, K. at el. J. Biol. Chem. 2001, 276, 31305-31310. Sato, M. et al. Nat. Biotech. 2002, 20, 287-294; Violin, J. D. et al. J. Cell Biol. 2003, 161, 899-909), or Ca²⁺ chelation between the phosphate and internal chelator causing disruption of PET-quenching (Chen, C.-A.; et al. J. Am. Chem. Soc. 2002, 124, 3840-3841). A majority of these sensors have very modest fluorescence increases or sometimes decreases, with the notable exception of 1.5-2.5-fold increases in the probes reported by Lawrence and coworkers (Chen 2002, supra; Yeh, R.-H.; et al. J. Biol. Chem. 2002, 277, 11527-11532; Wang, Q.; et al. J. Am. Chem. Soc. 2006, 128, 1808-1809). However, these types of probes, with fluorophores adjacent to the phosphorylated residue or using very large fluorophores, may interfere with their recognition by and reactivity with certain kinases.

### SUMMARY OF THE INVENTION

The present invention provides metal-binding analytical reagents that exhibit chelation-enhanced fluorescence (CHEF) upon binding to Mg²⁺. The present invention further provides methods of using the reagents for detecting the presence of or quantifying the activity of individual kinases or groups of kinases in a sample. The corresponding phosphopeptides can also be used as substrates to monitor the activity of protein phosphatases, where removal of the phosphate group on the targeted serine, threonine or tyrosine residue will result in a reduction in fluorescence.

### BRIEF DESCRIPTION OF THE FIGURES

FIG. 1 is a graph of soluble tyrosine kinase (Soluble-TK) activity.

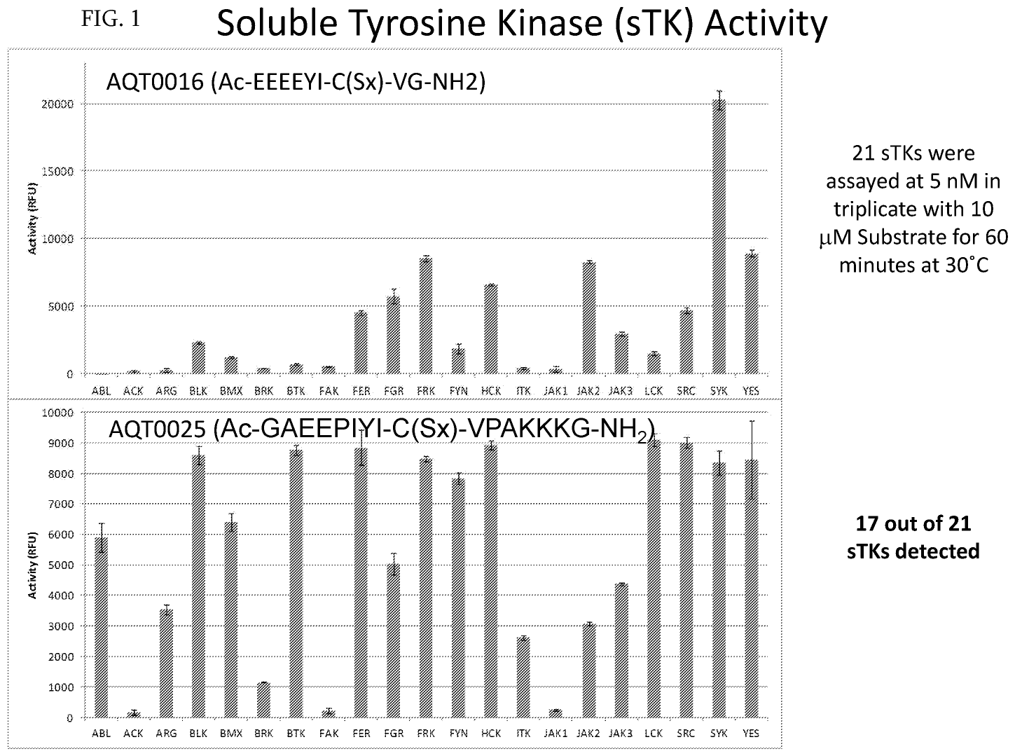

FIG. 2 is a graph of receptor tyrosine kinase (RTK) activity.

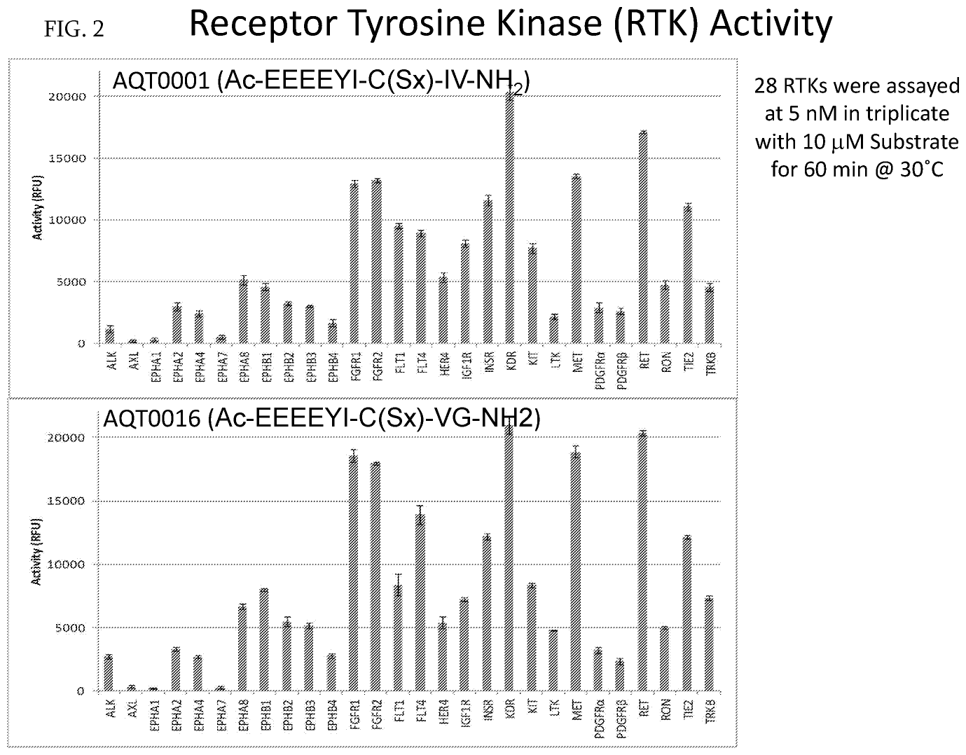

FIG. 3 is a graph of receptor tyrosine kinase activity dramatic improvement in selectivity after 1 round of optimization.

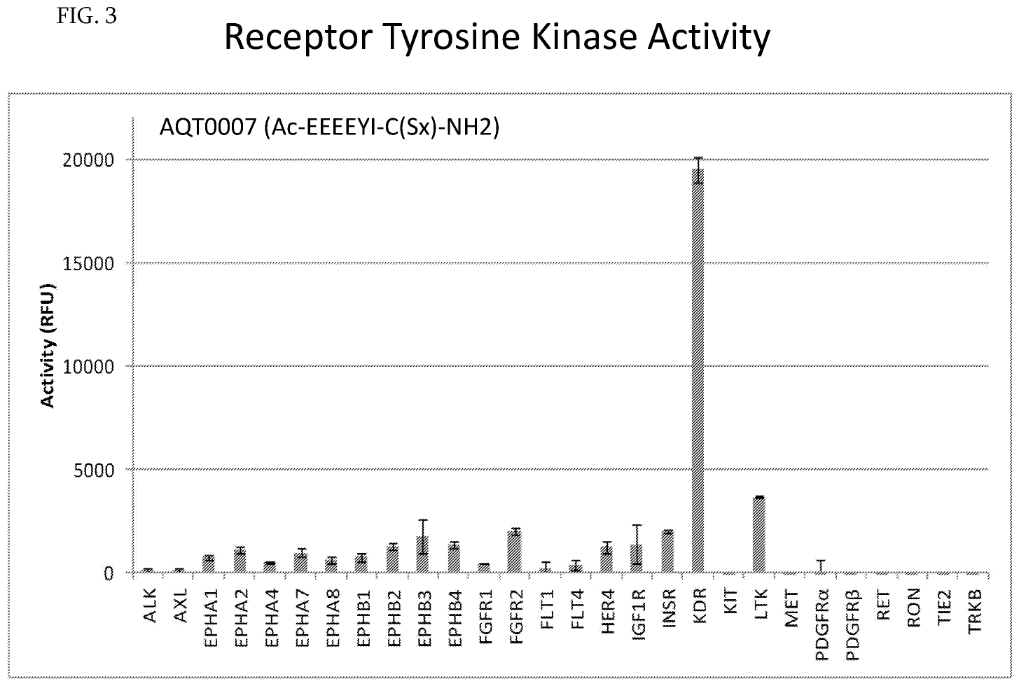

FIG. 4 is a graph of screen of kinase activity with Omnia Y12 substrate.

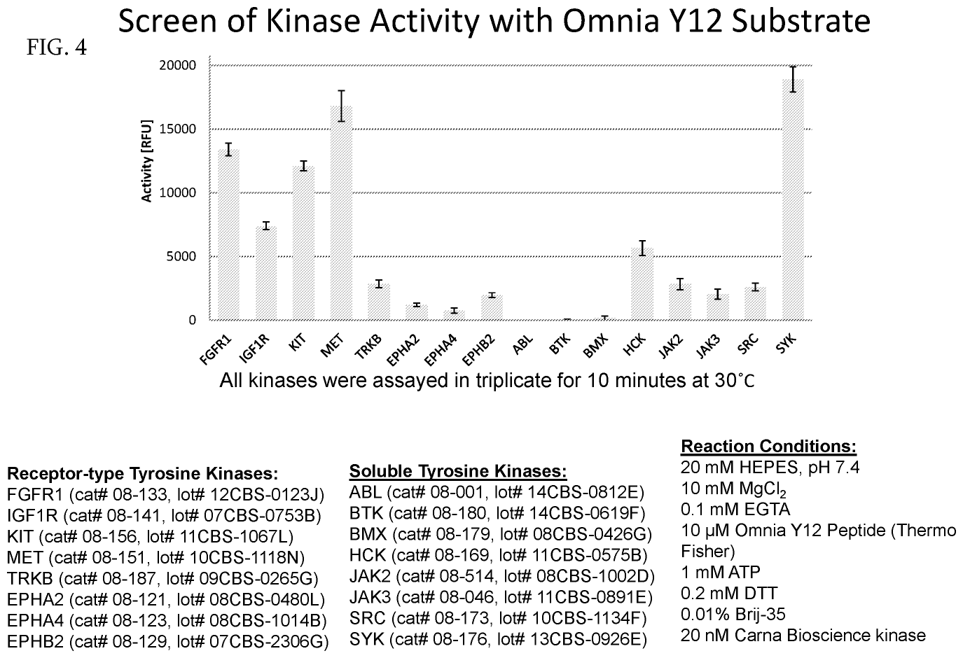

FIG. 5 is a graph of receptor tyrosine kinase (RTK) activity.

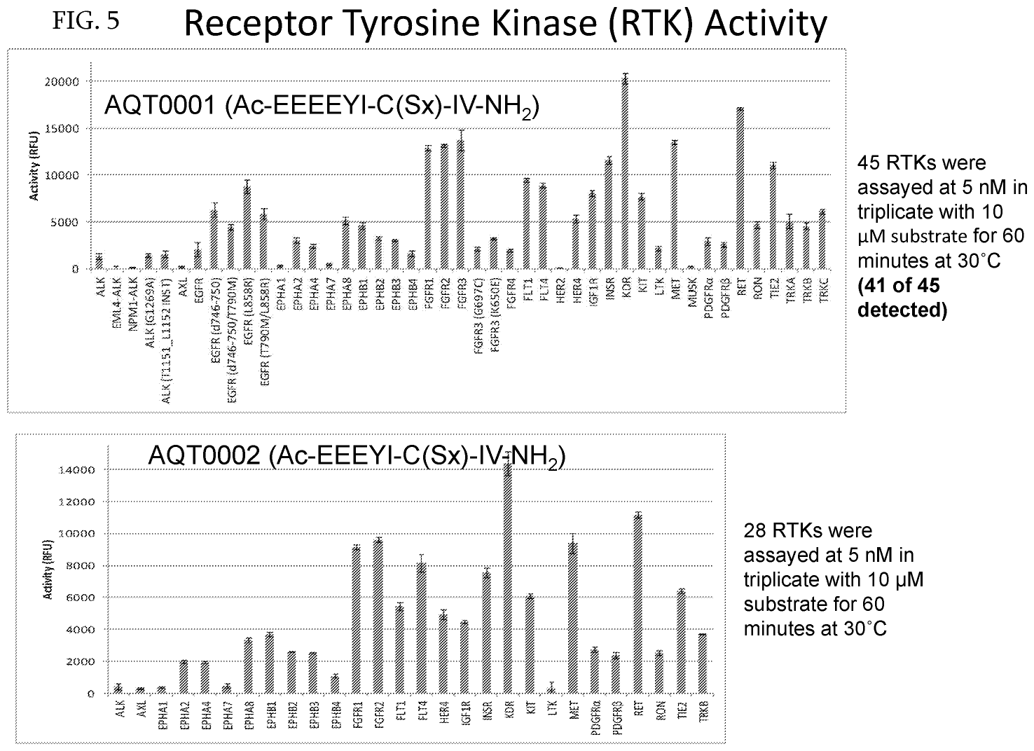

FIG. 6 is a graph of receptor tyrosine kinase (RTK) activity.

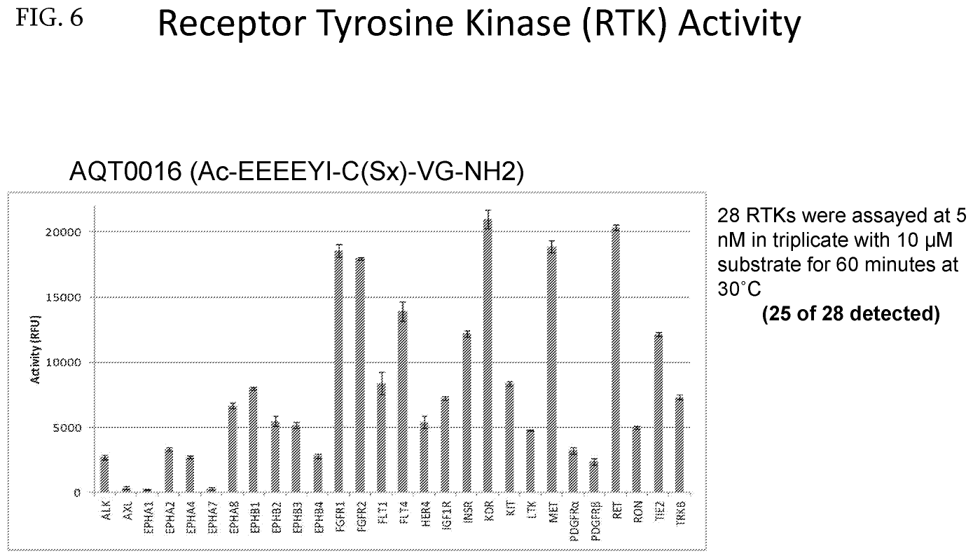

FIG. 7 is a graph of receptor tyrosine kinase (RTK) activity.

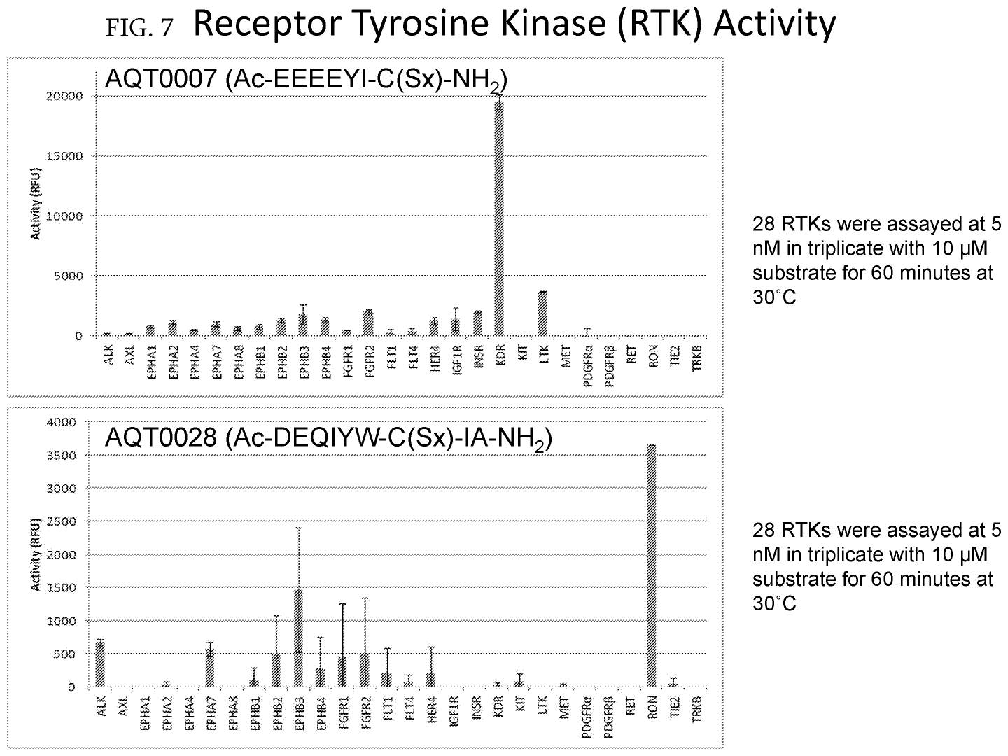

FIG. 8 is a graph of receptor tyrosine kinase (RTK) activity.

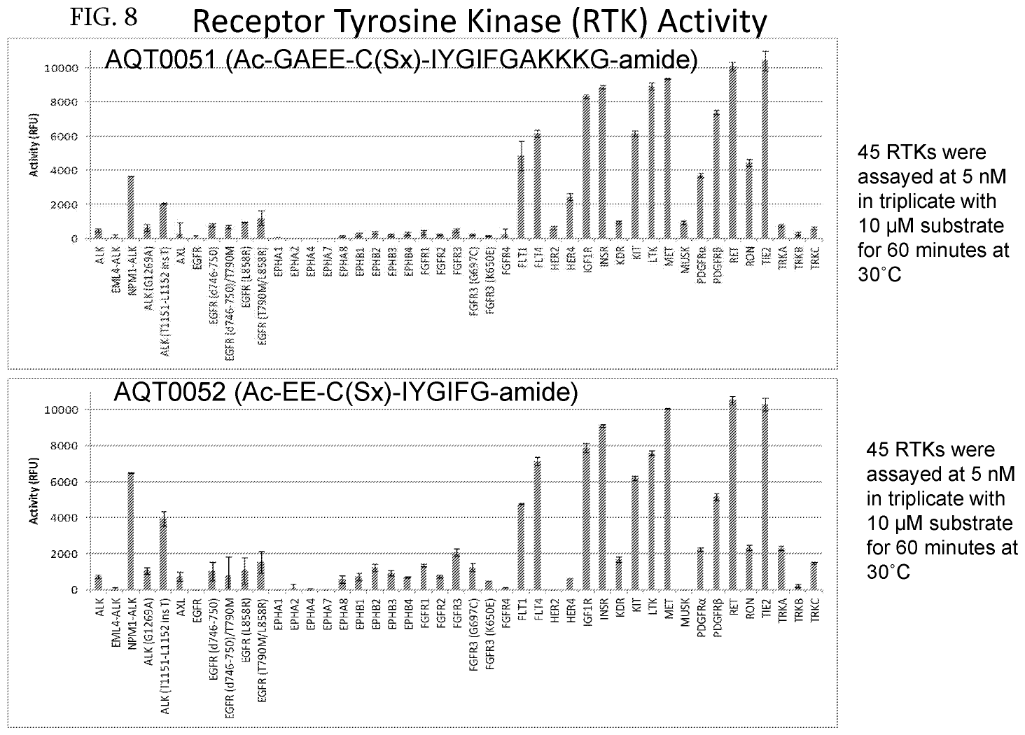

FIG. 9 is a graph of receptor tyrosine kinase (RTK) activity.

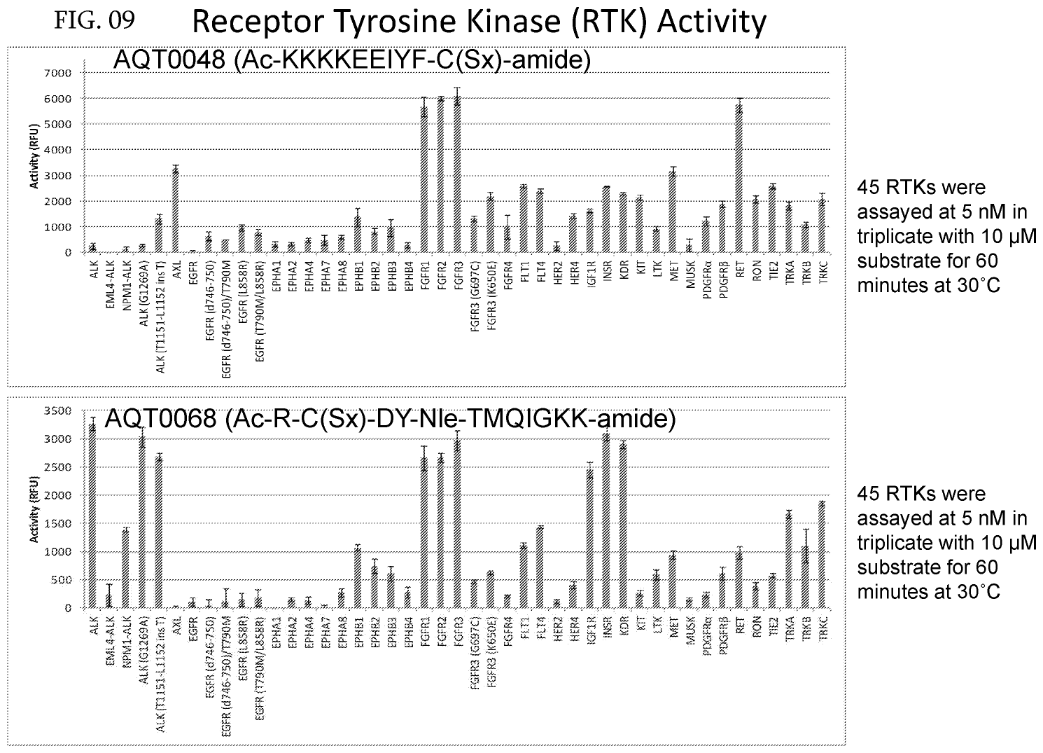

FIG. 10 is a graph of receptor tyrosine kinase (RTK) activity.

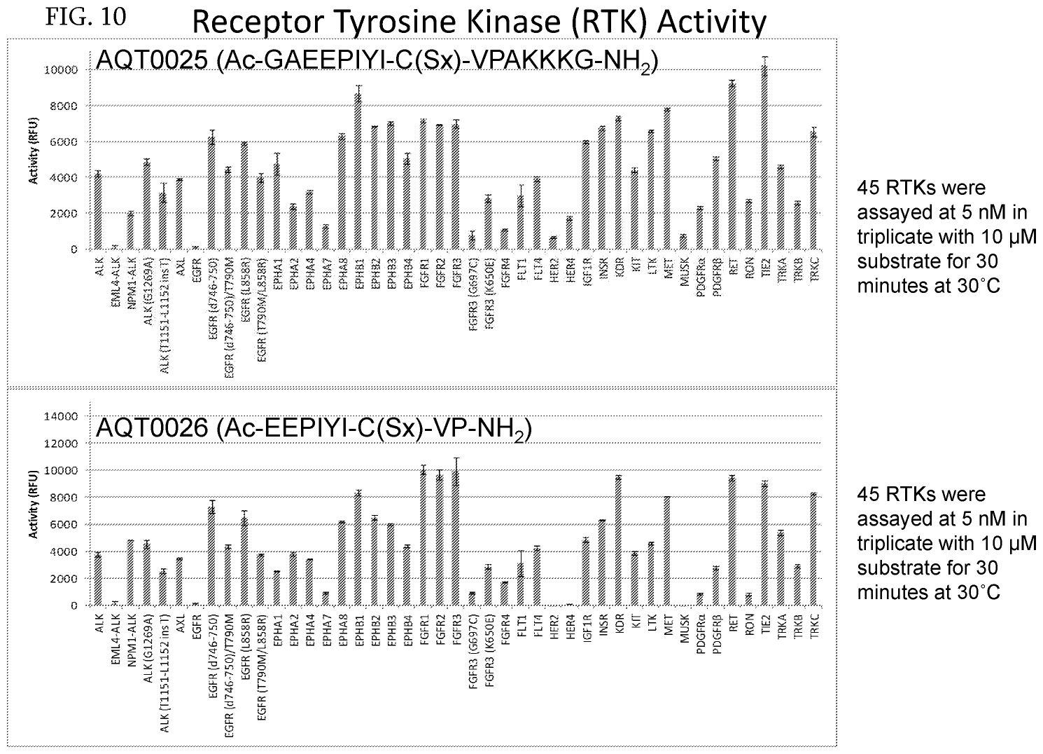

FIG. 11 is a graph of receptor tyrosine kinase (RTK) activity.

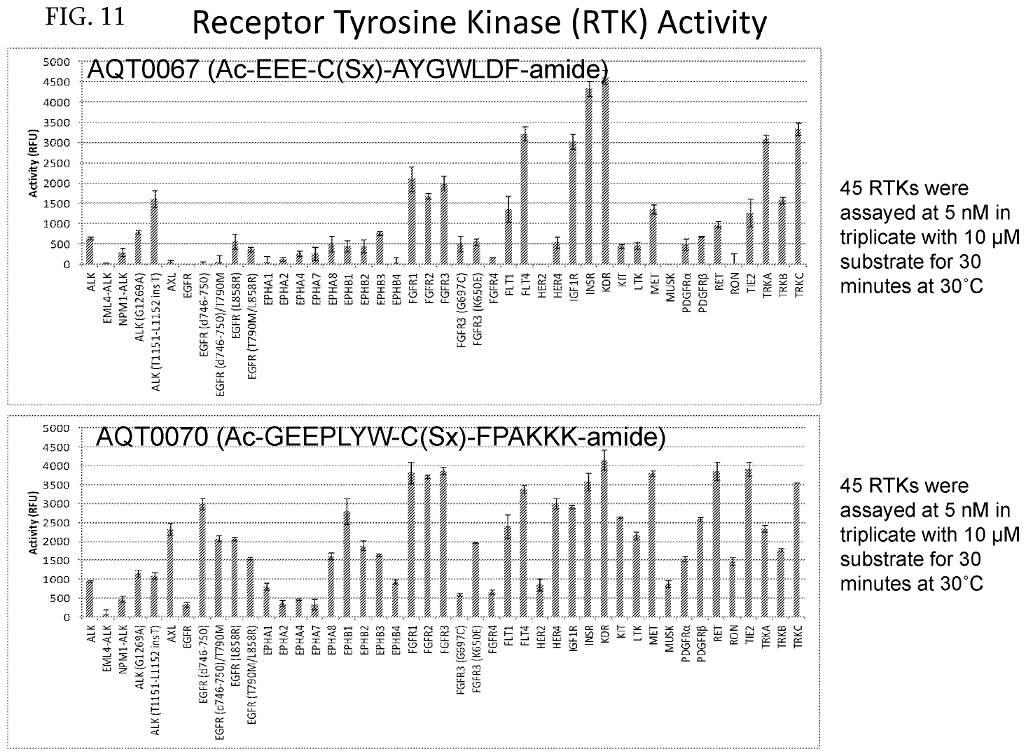

FIG. 12 is a graph of receptor tyrosine kinase (RTK) activity.

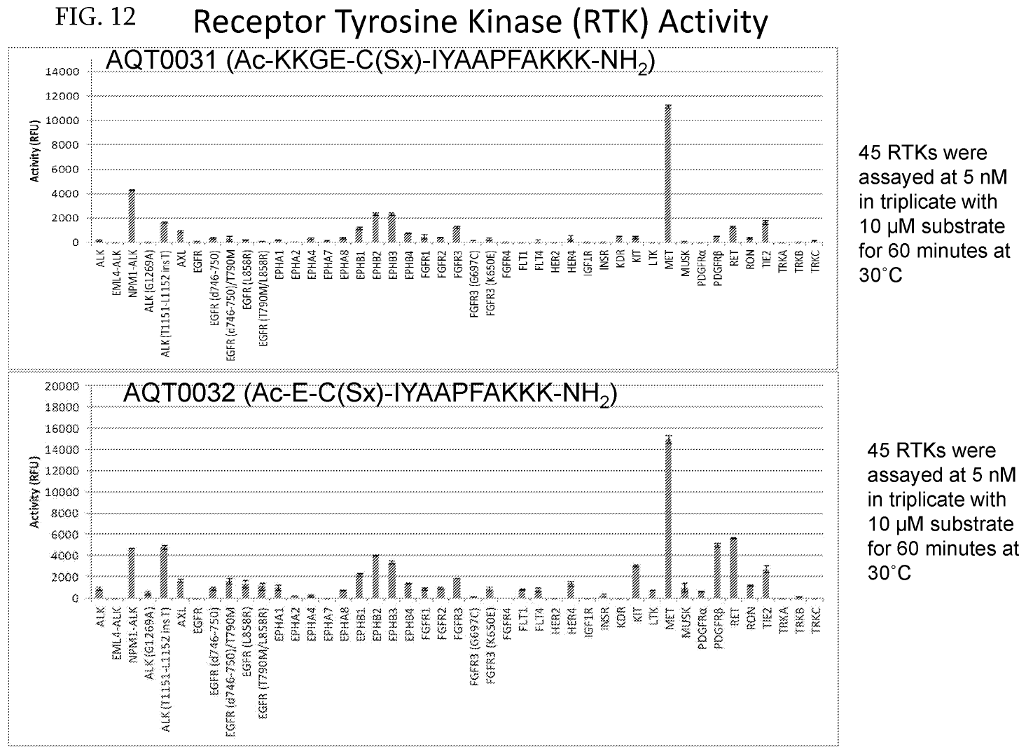

FIG. 13 is a graph of receptor tyrosine kinase (RTK) activity.

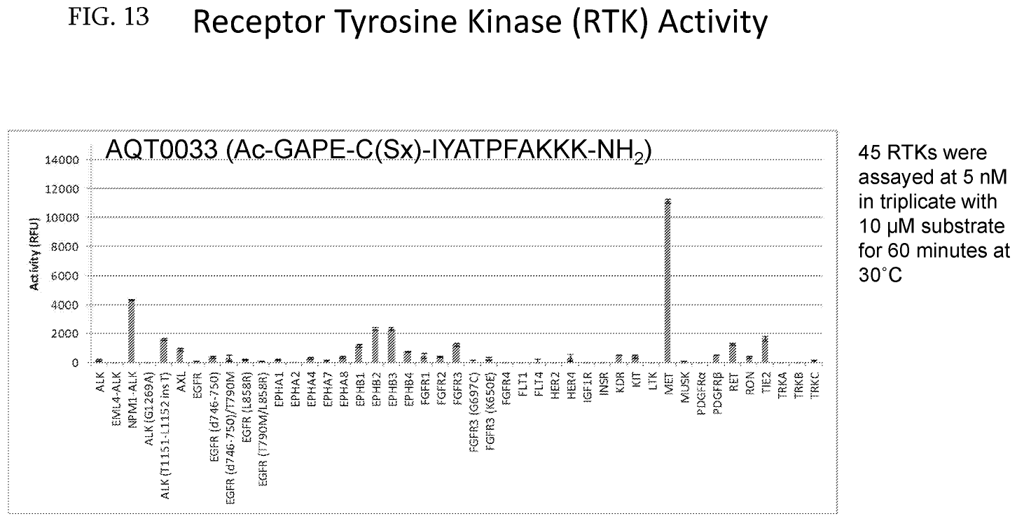

FIG. 14 is a graph of soluble tyrosine kinase (Soluble-TK) activity.

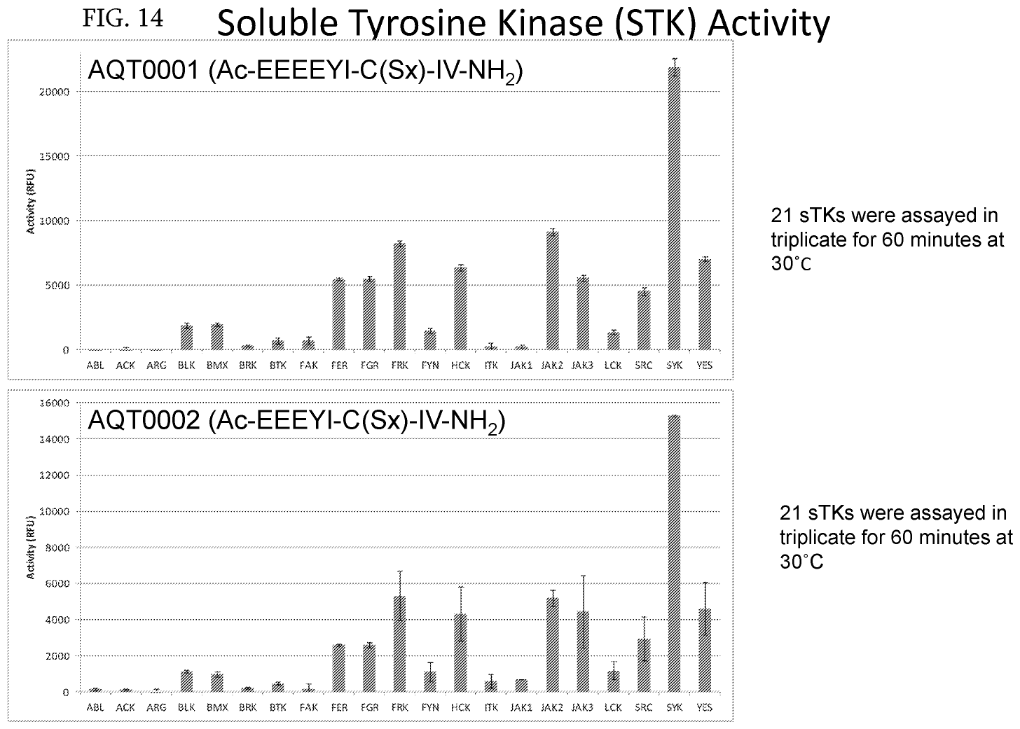

FIG. 15 is a graph of soluble tyrosine kinase (Soluble-TK) activity.

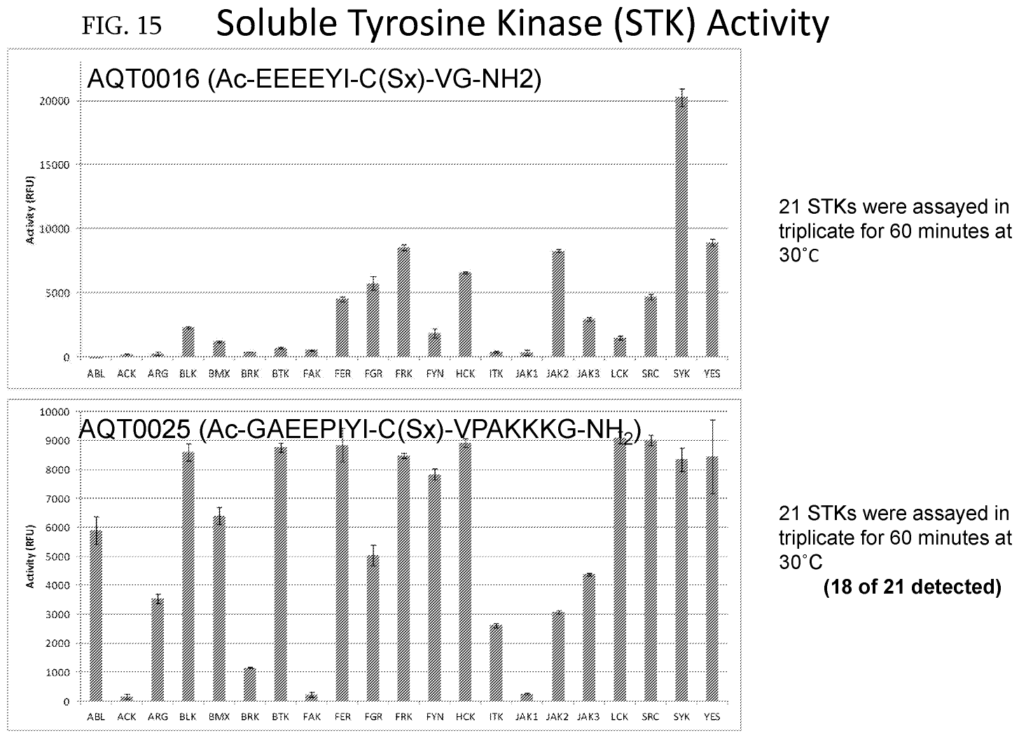

FIG. 16 is a graph of soluble tyrosine kinase (Soluble-TK) activity.

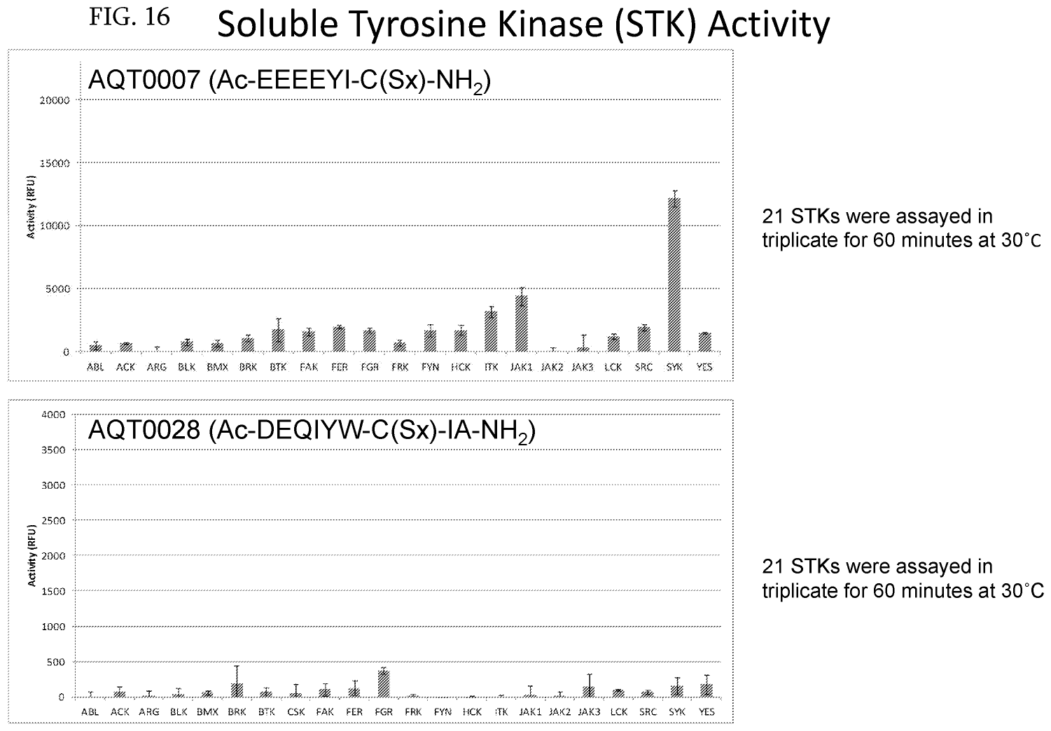

FIG. 17 is a graph of soluble tyrosine kinase (Soluble-TK) activity.

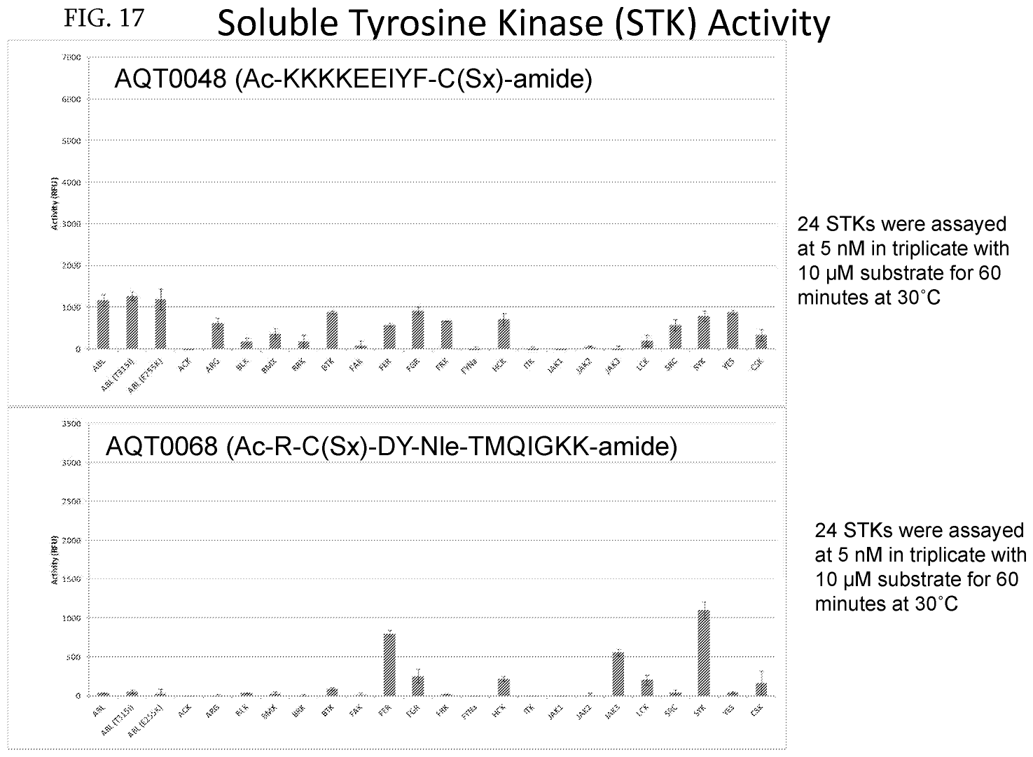

FIG. 18 is a graph of soluble tyrosine kinase (Soluble-TK) activity.

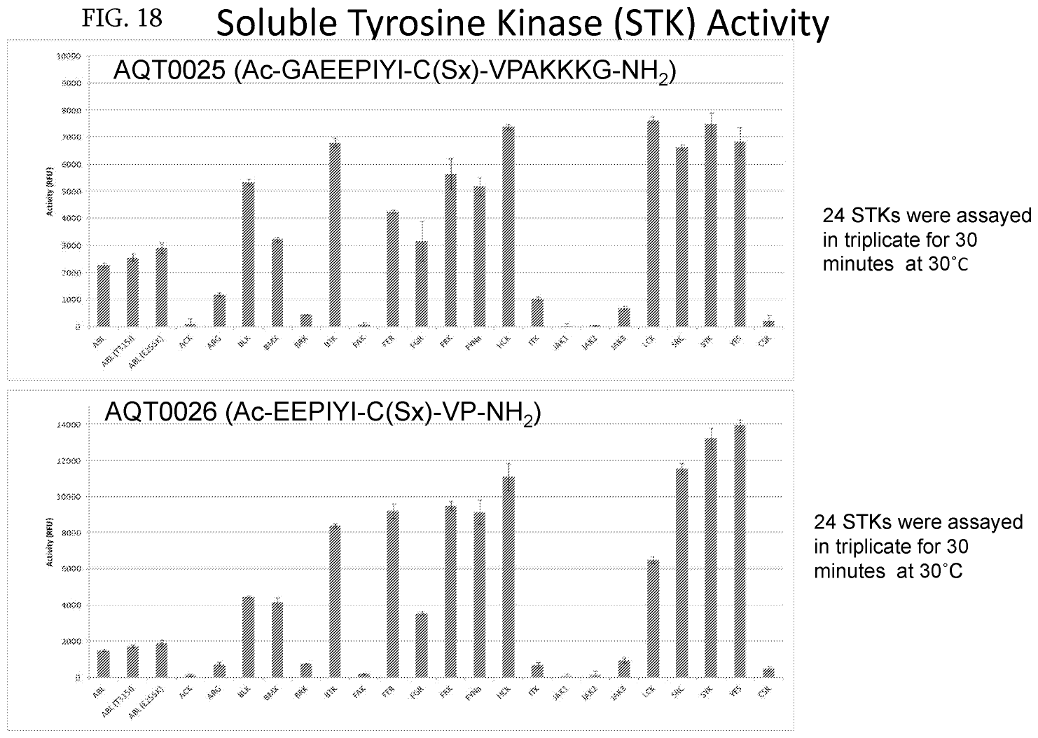

FIG. 19 is a graph of soluble tyrosine kinase (Soluble-TK) activity.

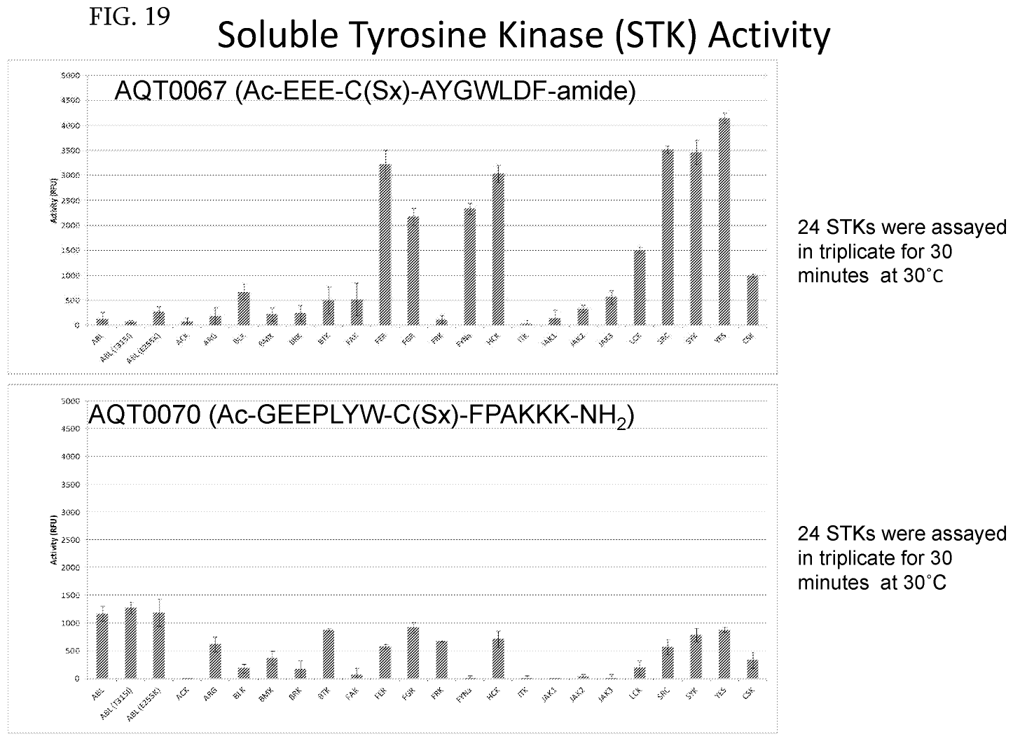

FIG. 20 is a graph of soluble tyrosine kinase (Soluble-TK) activity.

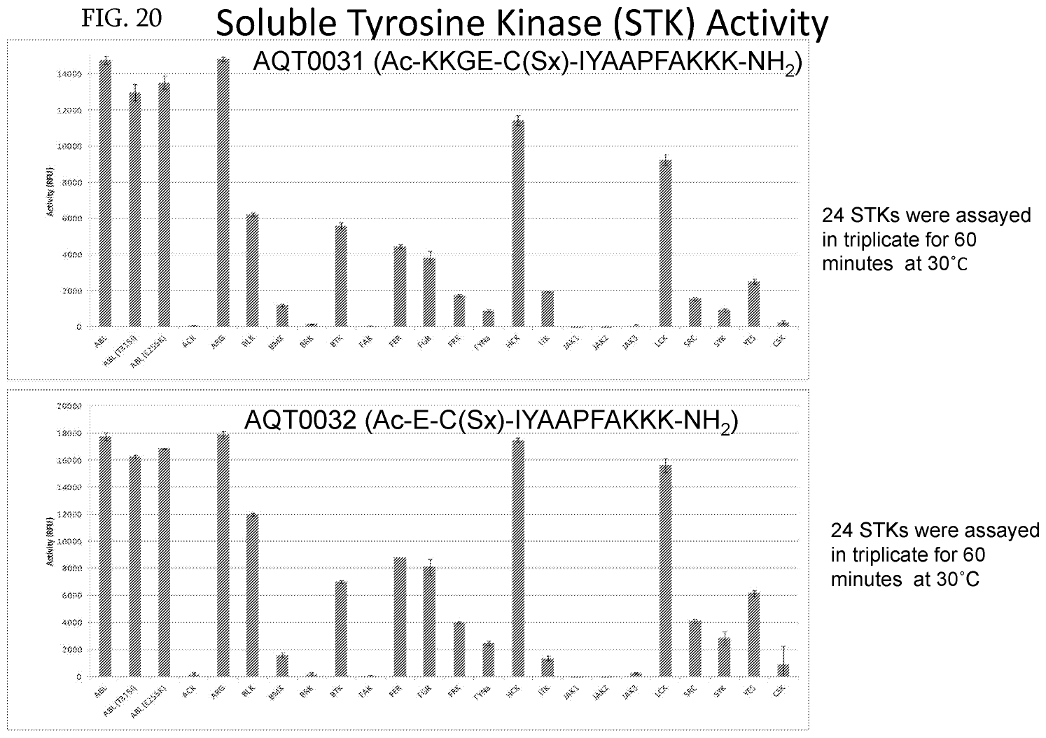

FIG. 21 is a graph of soluble tyrosine kinase (Soluble-TK) activity.

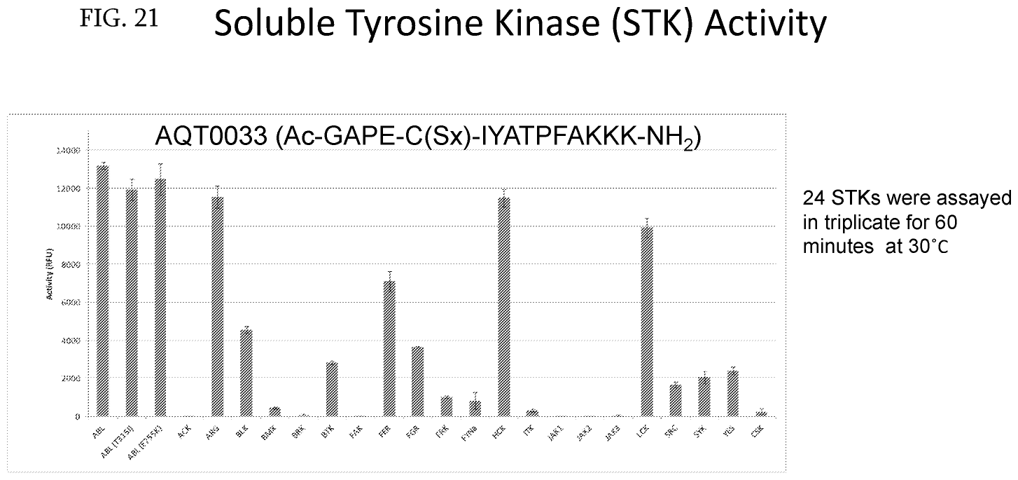

FIG. 22 is a graph of tyrosine kinase activity for AQT0001 (Ac-EEEEYI-C(Sx)-IV-NH₂).

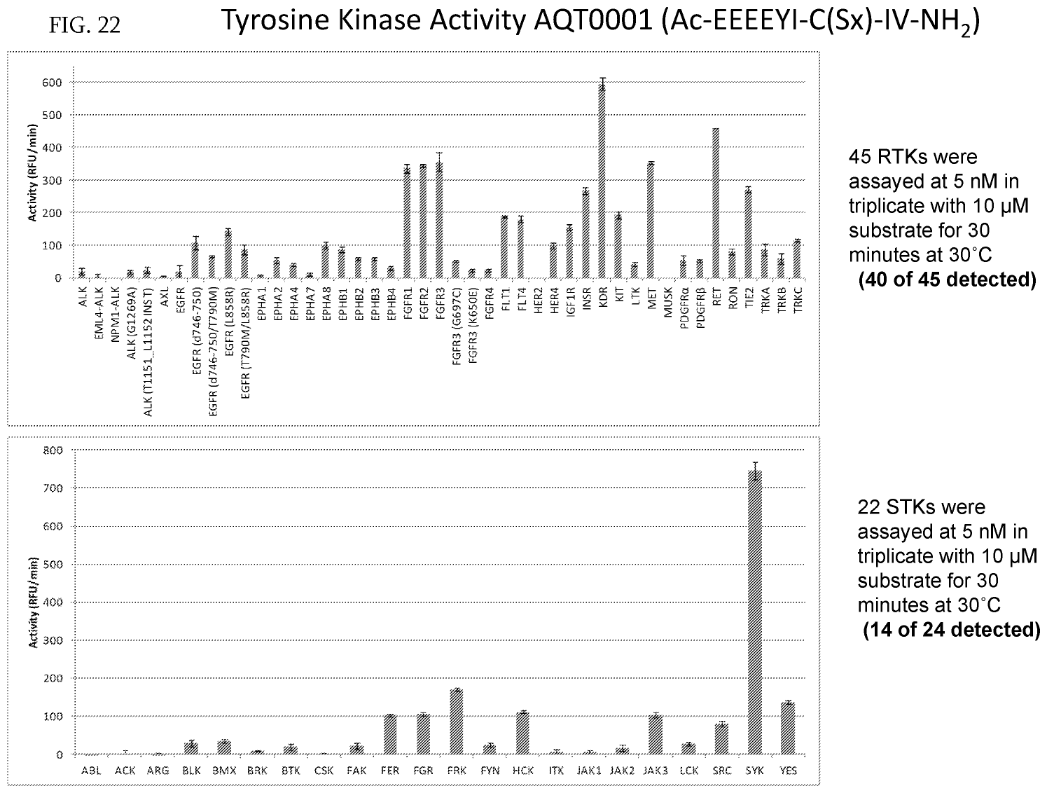

FIG. 23 is a chart of receptor tyrosine kinase (RTK) activity for AQT0001.

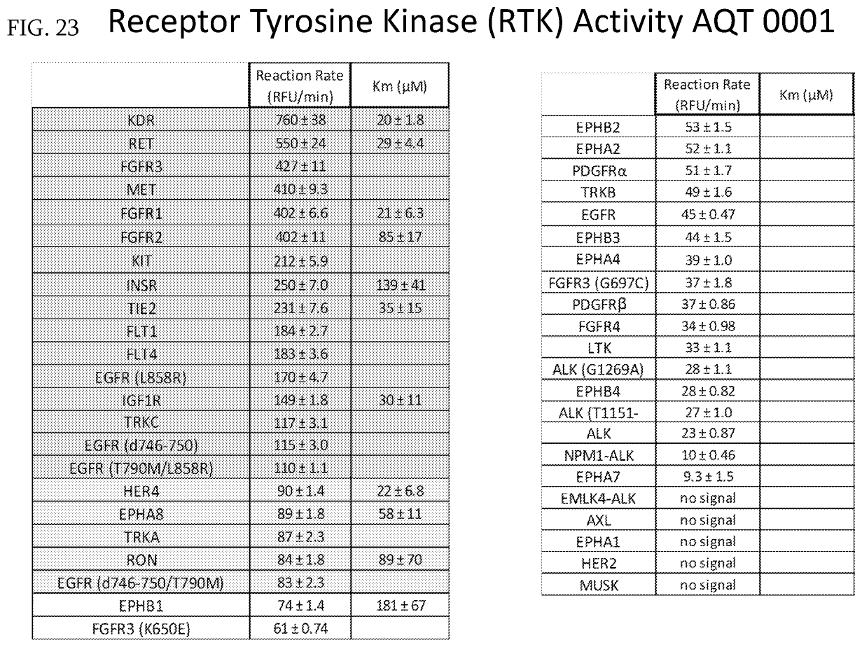

FIG. 24 is a chart of soluble tyrosine kinase (Soluble-TK) activity for AQT0001.

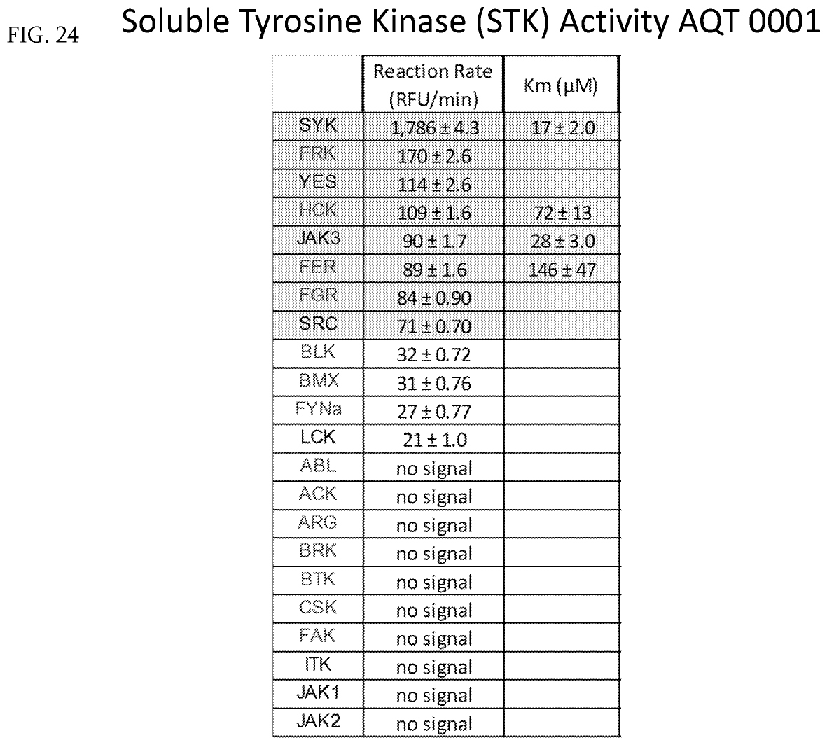

FIG. 25 is a graph of tyrosine kinase activity for AQT0032 (Ac-E-C(Sx)-IYAAPFAKKK-NH₂).

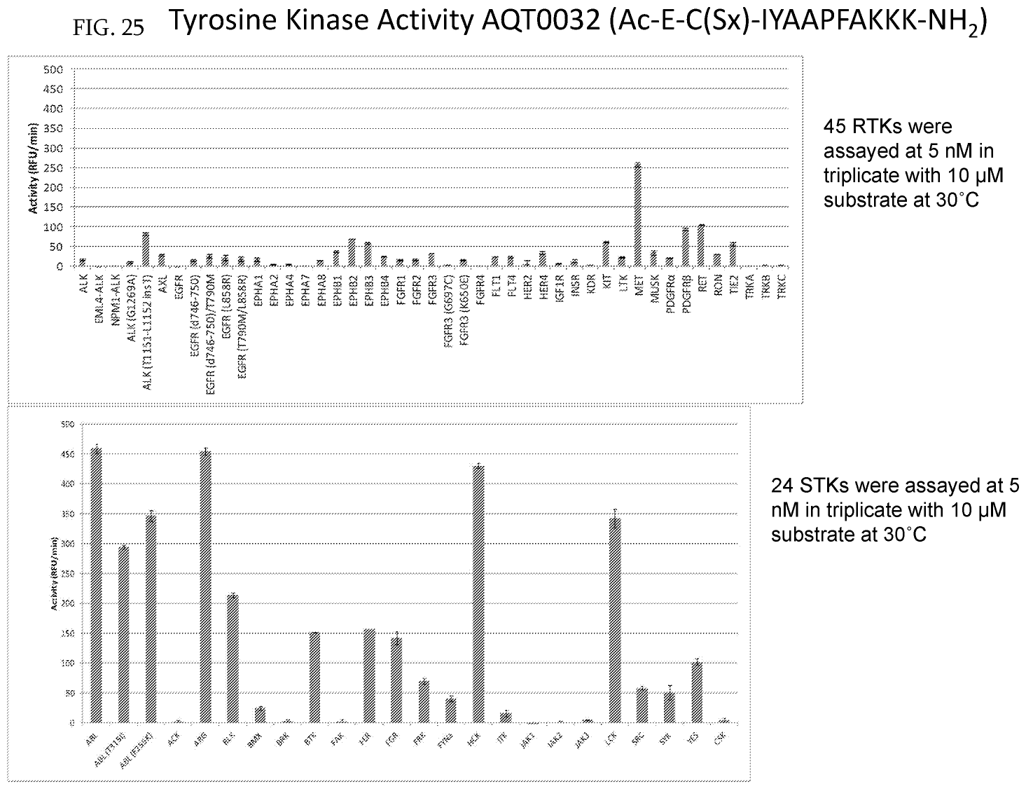

FIG. 26 is a chart of receptor tyrosine kinase (RTK) activity for AQT0032.

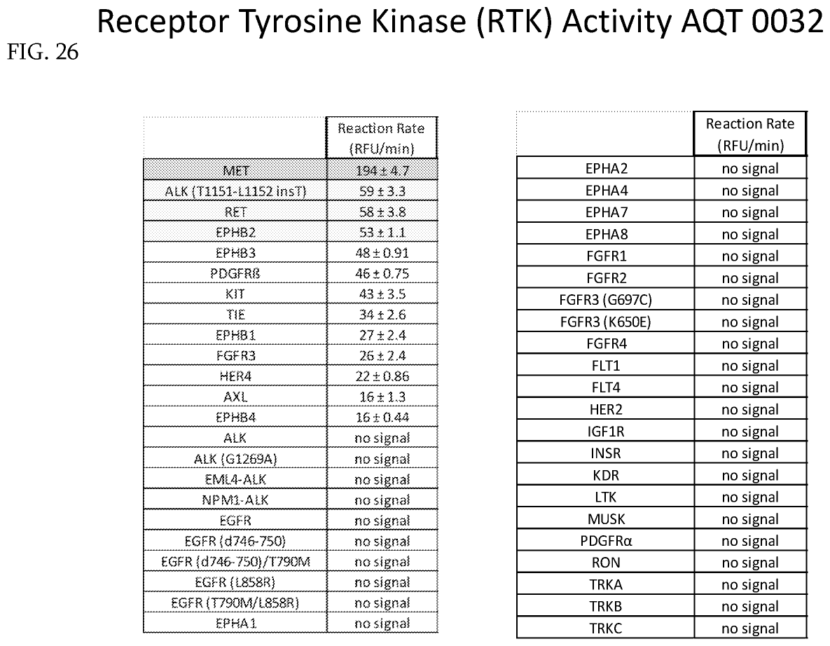

FIG. 27 is a chart of soluble tyrosine kinase (Soluble-TK) activity for AQT0032.

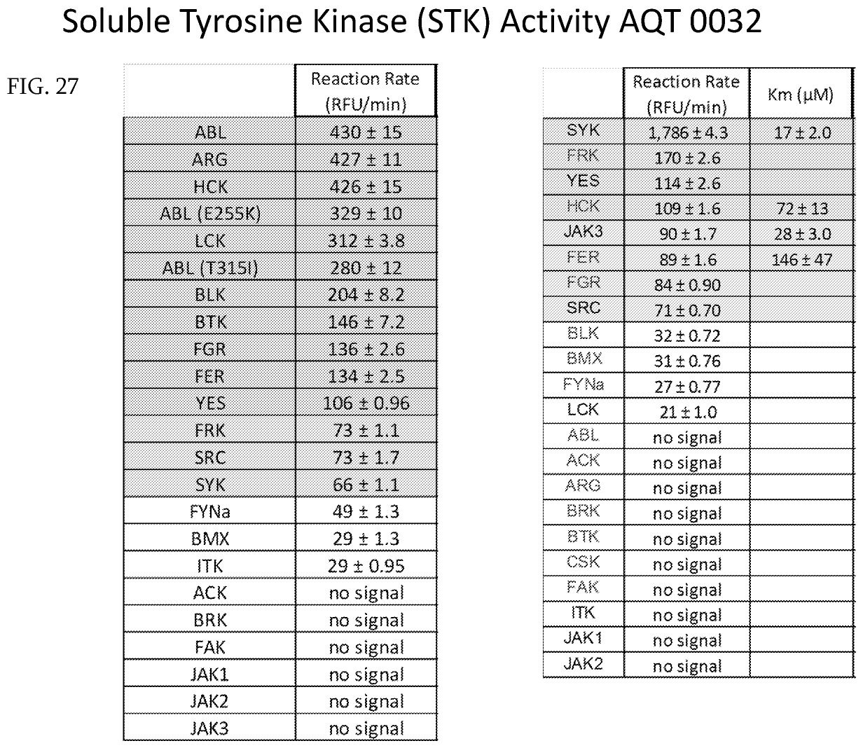

FIG. 28 is a graph of AGC Serine/Threonine kinase activity.

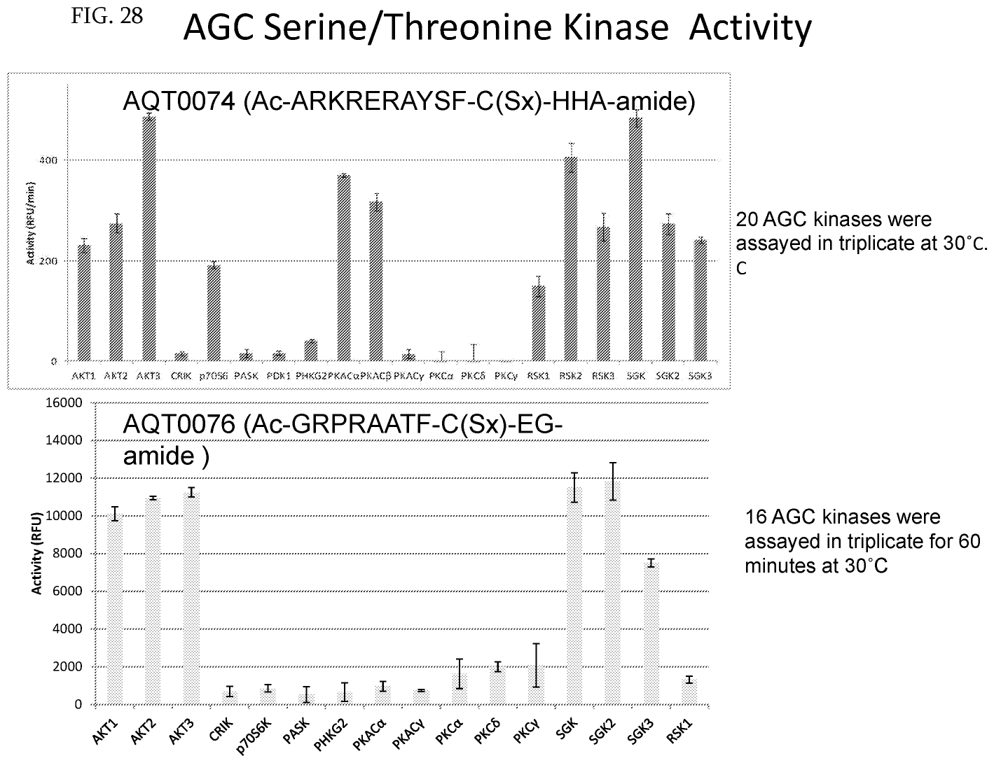

FIG. 29 is a graph of CMGC Serine/Threonine kinase activity.

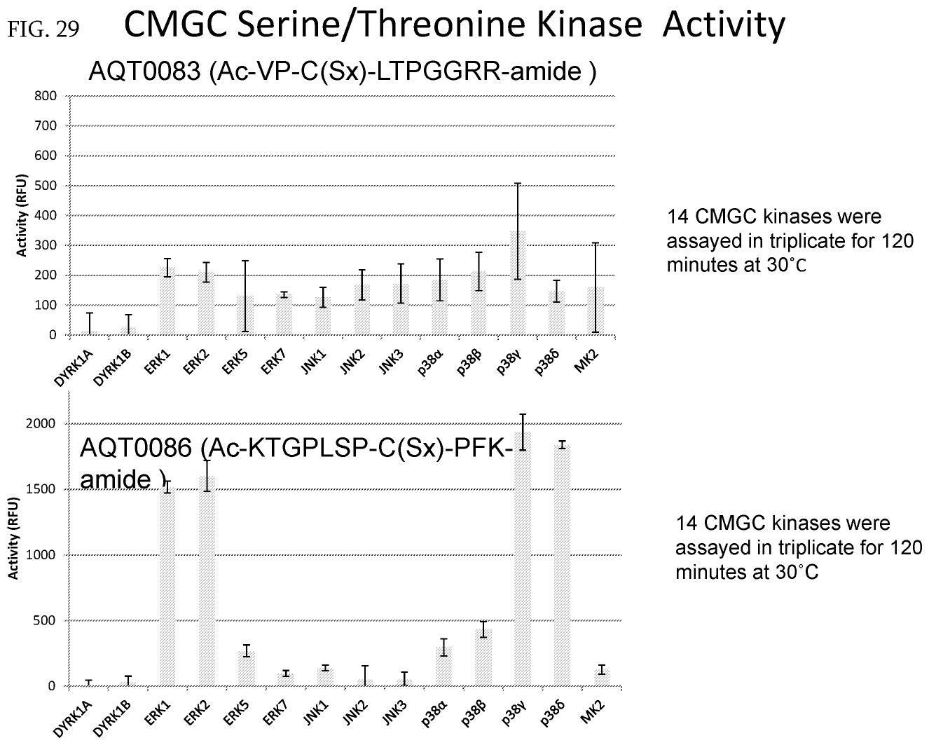

FIG. 30 is a graph of CMGC Serine/Threonine kinase activity.

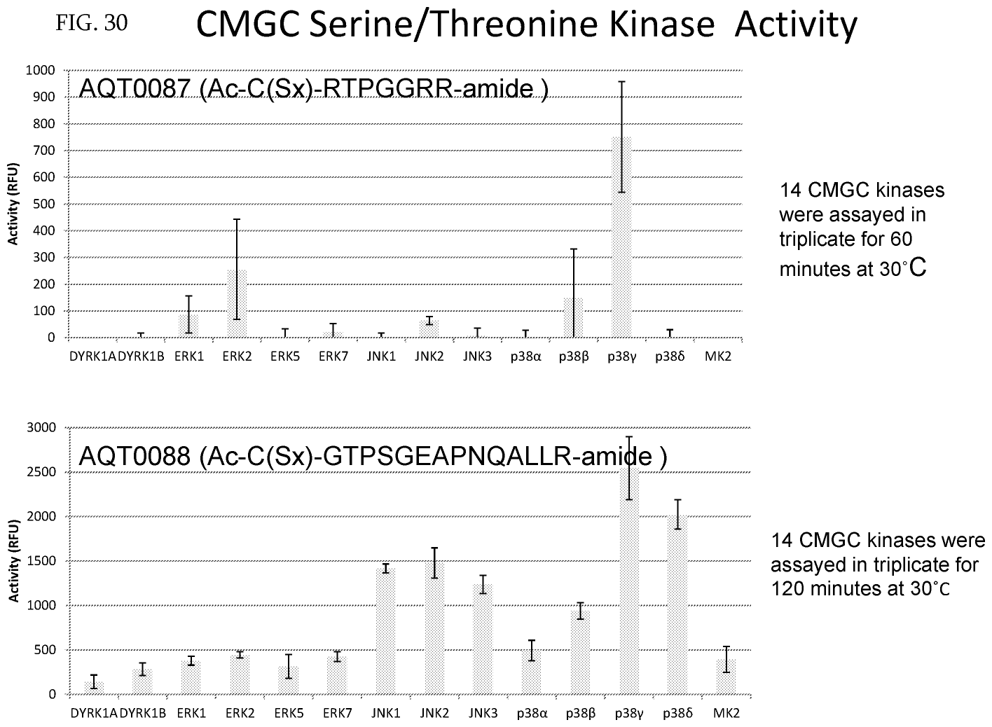

FIG. 31 is a graph of CMGC Serine/Threonine kinase activity.

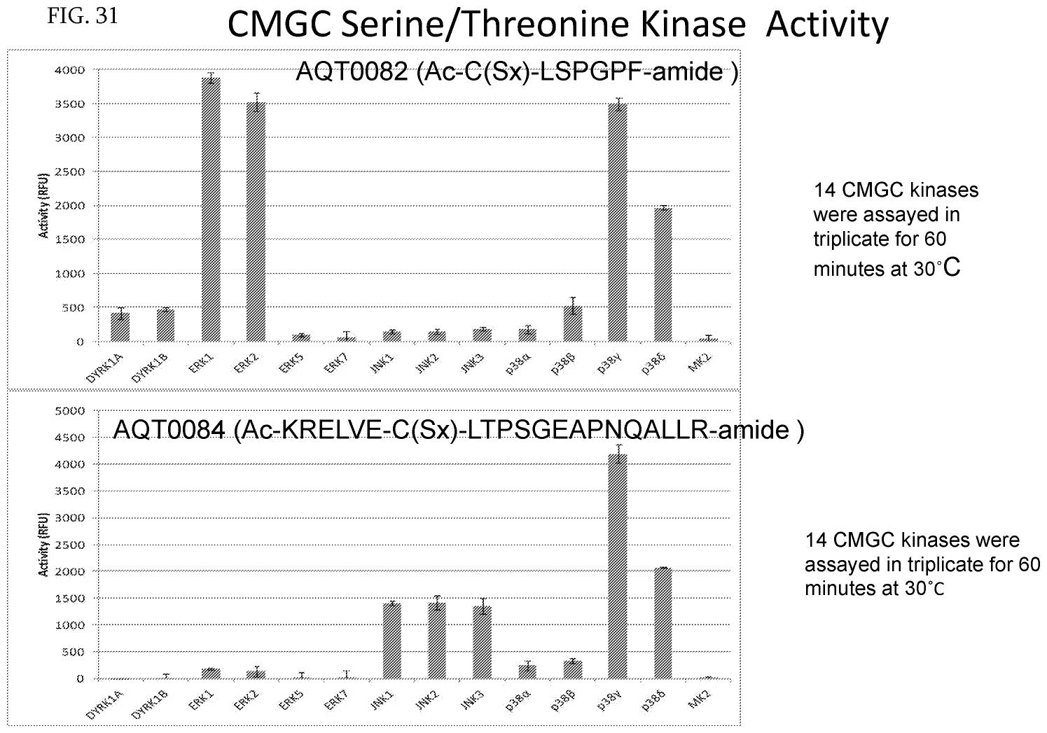

FIG. 32 is a chart of AGC Serine/Threonine kinase activity for AQT0074 (Ac-ARKRERAYSF-C(Sx)-HHA-amide).

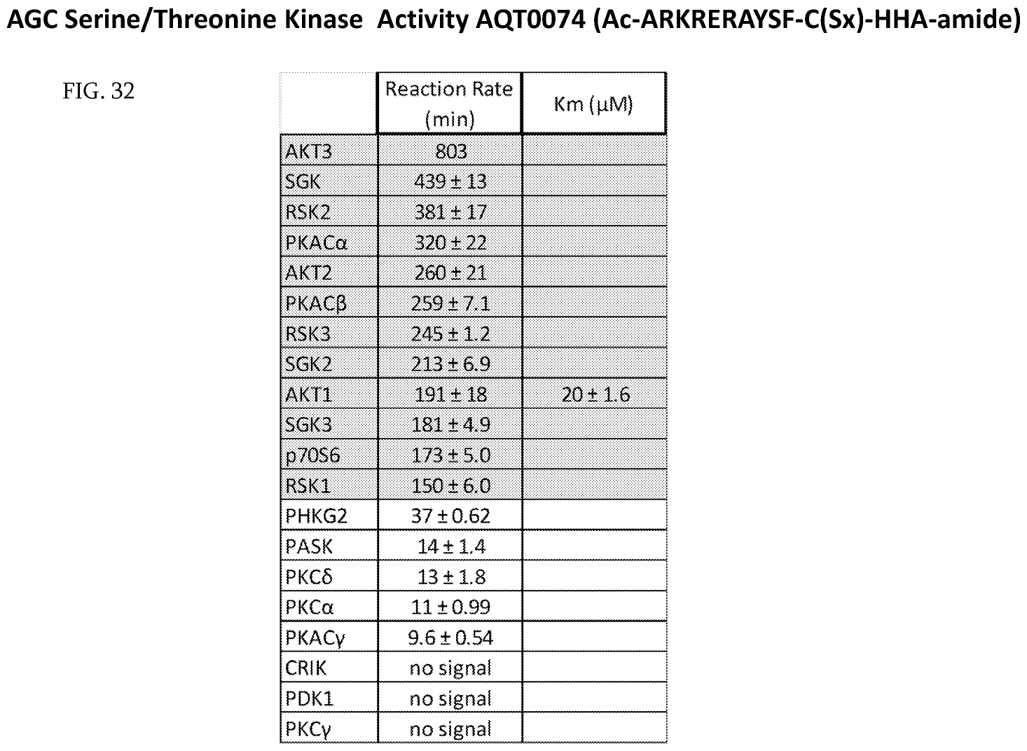

FIG. 33 is a chart of AKT1, 2 & 3 Serine/Threonine Kinase Activity Measured Using AQT's Panel of Substrates.

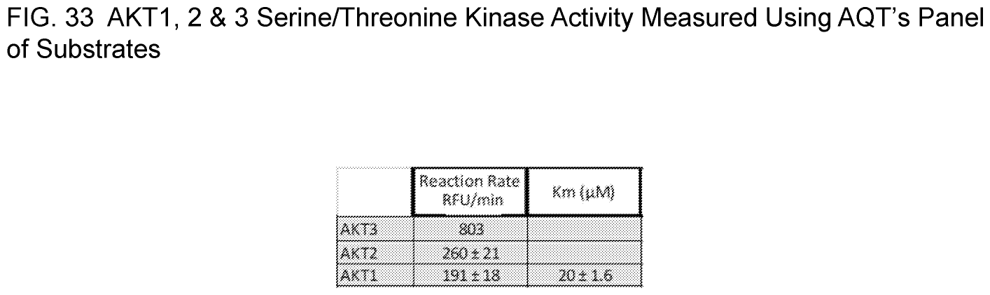

FIG. 34 is a chart of EGFR Substrate Optimization (Mutant EGFRs).

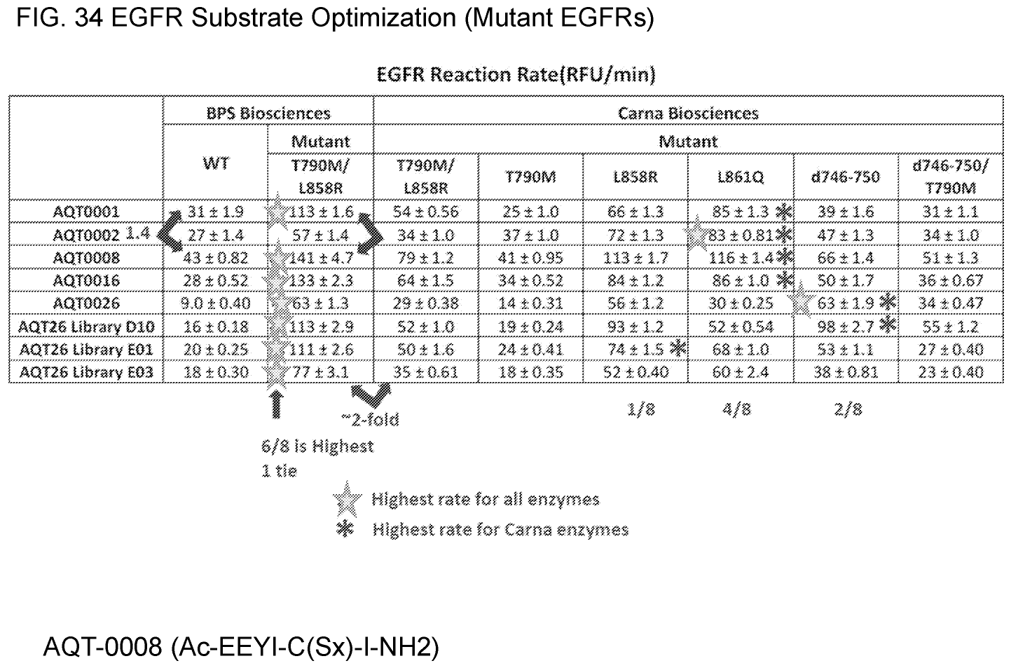

FIG. 35 is a chart of Broad AGC Serine/Threonine Kinase Activity Measured Using PKA-S4 Including SGK1/2/3 and PRKACA/B.

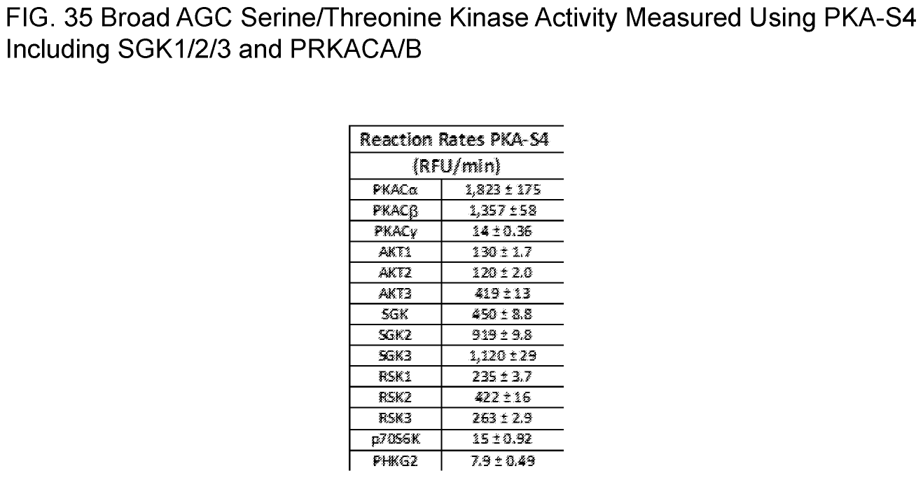

FIG. 36 is a chart of Substrates for SGK1, SGK2, SGK3 Identified from AQT's CSx Panel.

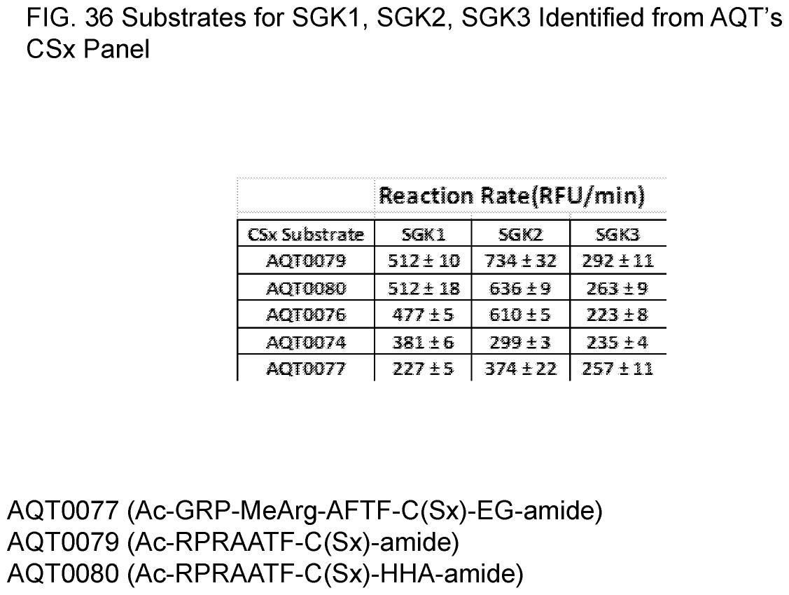

FIG. 37 is a chart FGFR Substrate Screening with Wild-type & Mutant FGFRs

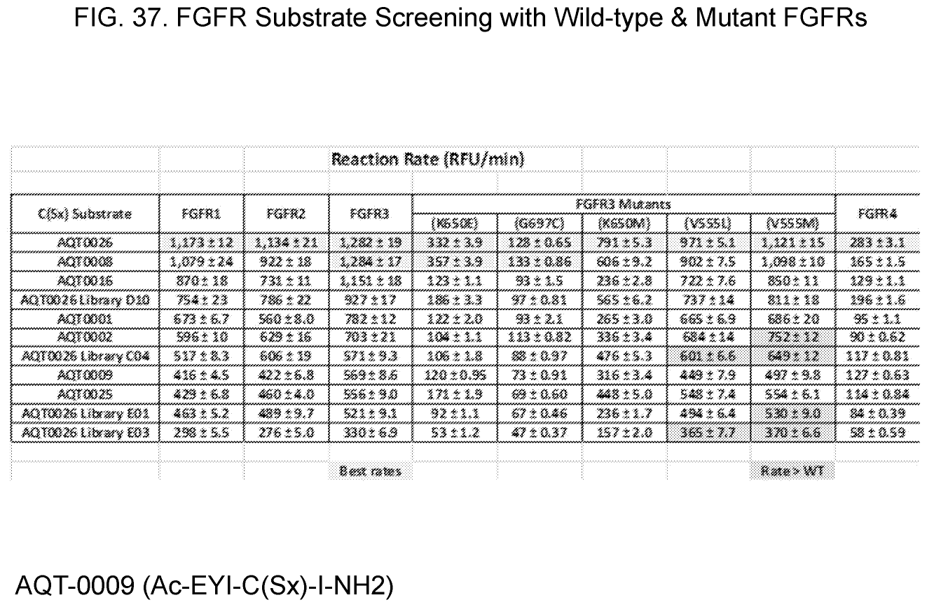

### DETAILED DESCRIPTION OF THE INVENTION

The present invention provides metal-binding analytical reagents that are useful for detecting and quantifying kinase activities. The metal binding compounds of the invention are peptidyl sensors which include a metal-binding peptide of the present invention and at least one kinase recognition sequence comprising an amino acid (e.g., serine, threonine, or tyrosine) that can be phosphorylated in the presence of a kinase.

The present invention provides superior Sox-based substrates by allowing for flanking sequence recognition determinants on either side of the Sox moiety/phosphorylation site. This approach is used to generate both highly-generic substrates (for use with a range of purified kinases) or highly-selective substrates (for use with unfractionated cell or tissue lysates).

U.S. Pat. No. 7,262,282 (incorporated by reference) discloses linear Sox peptide sensors that include a metal binding amino acid residue and a kinase recognition sequence with a hydroxyamino acid that can be phosphorylated in the presence of a kinase. The metal-binding amino acid residue is located on either side (N-terminally or C-terminally) of the hydroxyamino acid and is preferably separated from that recognition sequence by an amino acid or peptide that is capable of assuming a β-turn conformation (“a β-turn sequence”). In some cases, the β-turn sequence is separated from the hydroxyamino acid by one or more amino acids.

U.S. Pat. No. 7,964,729 (incorporated by reference) discloses metal binding compounds that exhibit chelation-enhanced fluorescence (CHEF) upon binding to Mg²⁺. This patent further provides peptidyl sensors which include a metal-binding component and at least one kinase recognition sequence with a hydroxyamino acid that can be phosphorylated by the kinase. The peptide sensors may also be used to detect sulfation of hydroxyamino acids.

### Definitions

The term “hydroxyl” or “hydroxy” means the —OH group.

The term “amino” means the —NR′R″ group, where R′ and R″ are each independently hydrogen or alkyl.

The term “halogen” means a chlorine, bromine, iodine, or fluorine atom.

The term “alkyl” means a hydrocarbon group that may be linear, cyclic, or branched or a combination thereof having the number of carbon atoms designated (i.e., C₁₋₈ means one to eight carbon atoms). Examples of alkyl groups include methyl, ethyl, n-propyl, isopropyl, n-butyl, t-butyl, isobutyl, sec-butyl, cyclohexyl, cyclopentyl, (cyclohexyl)methyl, cyclopropylmethyl, bicyclo[2.2.1]heptane, bicyclo[2.2.2]octane, etc. Alkyl groups can be substituted or unsubstituted, unless otherwise indicated. Examples of substituted alkyl groups include haloalkyl, thioalkyl, aminoalkyl, and the like.

The term “substituent” means an atom or a group that replaces another atom or group in a molecule.

The term “N-terminal protecting group” refers to a group that prevents undesirable reaction of the amino functional group during subsequent transformations, and includes, but is not limited to, benzyl, substituted benzyl, benzyloxycarbonyl (Cbz), tert-butoxycarbonyl (Boc), 9-fluorenylmethoxycarbonyl (Fmoc), trityl, N-veratyloxycarbonyl (N-Voc), N-allyloxycarbonyl (N-Alloc) and N-pentenoyl (N-Pent).

The term “C-terminal protecting group” refers to a group that prevents undesirable reaction of the carboxyl functional group and includes, but is not limited to, C₁₋₁₂ alkyl (e.g., tert-butyl) and C₁₋₁₂ haloalkyl.

The term “chelation-enhanced fluorescence (CHEF)” means fluorescence enhancement of a compound as a result of metal ion binding (chelation) to that compound.

The term “capping group” means a chemical group connected to the N- or C-terminus of a peptide to prevent the peptide from degrading or being otherwise inappropriately modified.

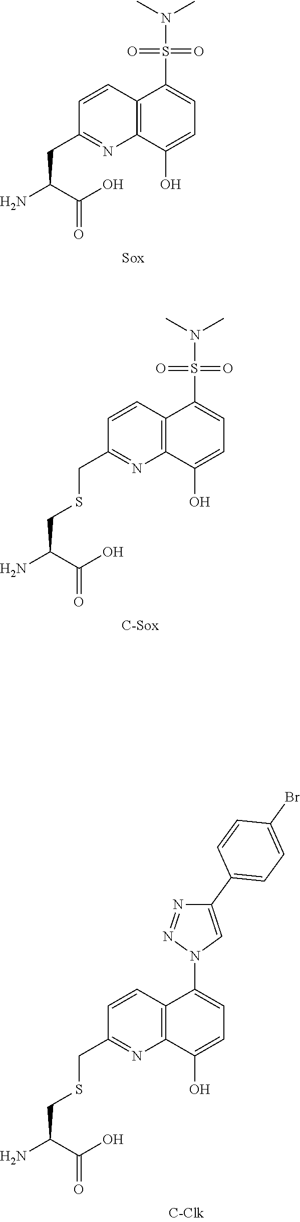 [SMILES: CN(C)S(=O)(=O)c1ccc(O)c2nc(C[C@H](N)C(=O)O)ccc12 ; CN(C)S(=O)(=O)c1ccc(O)c2nc(CSC[C@H](N)C(=O)O)ccc12 ; N[C@@H](CSCc1ccc2c(-n3cc(-c4ccc(Br)cc4)nn3)ccc(O)c2n1)C(=O)O]

C(Sx) represents an amino acid of formula (I), (II) or (III) as described below. C-Sox is a species of C(Sx).

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

S* represents S or (phospho)S; and
T* represents T or (phospho)T; and
Y* represents Y or (phospho)Y; and

### Compounds

The compounds of the present invention contain a metal binding moiety, referred to as the metal binding peptide, which comprises a metal-binding amino acid residue. In some embodiments, the compounds of the invention are oligopeptide comprising a fluorophore; and a (phospho)S, a (phospho)T, or a (phospho)Y residue at the +2 or −2 position relative to the fluorophore. In some embodiments, the position may be the +3 or −3 position. In some embodiments, a (phospho)S, a (phospho)T, or a (phospho)Y residue may be used at a certain position to facilitate kinase recognition and phosphorylation of the target amino acid (i.e., phospho-primed substrates).

In some embodiments, the metal-binding compound of the present invention is selected from the group consisting of: X-EEEEY*I-C(Sx)-IV-Z (SEQ ID NO: 1); X-EEEY*I-C(Sx)-IV-Z (SEQ ID NO: 2), X-EEY*I-C(Sx)-IV-Z (SEQ ID NO: 3), X-EY*I-C(Sx)-IV-Z (SEQ ID NO: 4), X-Y*I-C(Sx)-IV-Z (SEQ ID NO: 5), X-EEEEY*I-C(Sx)-I-Z (SEQ ID NO: 6), X-EEEY*I-C(Sx)-I-Z (SEQ ID NO: 7), X-EEEEY*I-C(Sx)-Z (SEQ ID NO: 8), X-EEY*I-C(Sx)-I-Z (SEQ ID NO: 9), X-EY*I-C(Sx)-I-Z (SEQ ID NO: 10), X-Y*I-C(Sx)-I-Z (SEQ ID NO: 11), X-EEEY*I-C(Sx)-Z (SEQ ID NO: 12), X-EEY*I-C(Sx)-Z (SEQ ID NO: 13), X-EY*I-C(Sx)-Z (SEQ ID NO: 14), and X-Y*I-C(Sx)-Z (SEQ ID NO: 15); wherein X is H or acetyl; and Z is OH or NH2.

In some embodiments, the metal-binding compound of the present invention is selected from the group consisting of: X-EEEIY*F-C(Sx)-dP-(2-Nal)-G-Z (SEQ ID NO: 16), X-EEEIY*F-C(Sx)-Z (SEQ ID NO: 17), X-EEIY*F-C(Sx)-Z (SEQ ID NO: 18), X-EIY*F-C(Sx)-Z (SEQ ID NO: 19), X-IY*F-C(Sx)-Z (SEQ ID NO: 20), X-Y*F-C(Sx)-Z (SEQ ID NO: 21), X-EEEEY*I-C(Sx)-dP-(2-Nal)-G-Z (SEQ ID NO: 22), X-EEEEY*I-C(Sx)-VG-Z (SEQ ID NO: 23), X-DPQEY*I-C(Sx)-LP-Z (SEQ ID NO: 24), X-EDEDY*E-C(Sx)-VG-Z (SEQ ID NO: 25), X-EAEAIY*A-C(Sx)-dP-(2-Nal)-G-Z (SEQ ID NO: 26), X-EAEAIY*A-C(Sx)-PF-Z (SEQ ID NO: 27), X-EAEAIY*A-C(Sx)-P-Z (SEQ ID NO: 28), X-EAEAIY*A-C(Sx)-Z (SEQ ID NO: 29), X-EEPIY*I-C(Sx)-VP-Z (SEQ ID NO: 30), X-DEQIY*W-C(Sx)-IA-Z (SEQ ID NO: 31), X-KKGEAIY*A-C(Sx)-dP-(2-Nal)-G-Z (SEQ ID NO: 32), X-KKGEAIY*A-C(Sx)-PFAKKK-Z (SEQ ID NO: 33), X-KKGE-C(Sx)-IY*AAPFAKKK-Z (SEQ ID NO: 34), X-E-C(Sx)-IY*AAPF-Z (SEQ ID NO: 35), X-E-C(Sx)-IY*A-Z (SEQ ID NO: 36), X-E-C(Sx)-IY*-Z (SEQ ID NO: 37), X-C(Sx)-IY*-Z (SEQ ID NO: 38), X-KKGEAIY*A-C(Sx)-PF-Z (SEQ ID NO: 39), X-PE-C(Sx)-IY*ATPG-Z (SEQ ID NO: 40), and X-PEVIY*A-C(Sx)-PG-Z (SEQ ID NO: 41); X-GAEEPIY*I-C(Sx)-VPAKKKG-Z (SEQ ID NO: 42) wherein X is H or acetyl; and Z is OH or NH2.

In some embodiments, the metal-binding compound of the present invention is selected from the group consisting of: X-KKKKEEIY*F-C(Sx)-Z (SEQ ID NO: 43), X-AEE-C(Sx)-IY*GELEA-Z (SEQ ID NO: 44), X-EE-C(Sx)-IY*GIFG-Z (SEQ ID NO: 45), X-C(Sx)-IY*ETDYYRKG-Z (SEQ ID NO: 46), X-EEEEY*I-C(Sx)-DYYRKG-Z (SEQ ID NO: 47), X-ELEDDY*E-C(Sx)-Z (SEQ ID NO: 48), X-EE-C(Sx)-DY*VEFKKK-Z (SEQ ID NO: 49), X-EEEEY*I-C(Sx)-FKKK-Z (SEQ ID NO: 50), X-RRRRRRSETDDY*A-C(Sx)-IIDEEDT-Z (SEQ ID NO: 51), X-RAFIY*A-C(Sx)-IP-Z (SEQ ID NO: 52), X-DDEPIY*A-C(Sx)-LADIT-Z (SEQ ID NO: 53), X-C(Sx)-IY*GVIE-Z (SEQ ID NO: 54), X-EEE-C(Sx)-AY*GWLDF-Z (SEQ ID NO: 55), X-C(Sx)-EY*VNIEFG-Z (SEQ ID NO: 56), X-GEEPLY*W-C(Sx)-FPAKKK-Z (SEQ ID NO: 57), X-ESSDDY*V-C(Sx)-Z (SEQ ID NO: 58), and X-RAHEEIY*H-C(Sx)-Z (SEQ ID NO: 59); wherein X is H or acetyl; and Z is OH or NH2.

In some embodiments, the metal-binding compound of the present invention is selected from the group consisting of: X-VYS-C(Sx)-DY*[pY]RLFNPS-Z (SEQ ID NO: 60), X-DE-C(Sx)-DY*[pY]EIP-Z (SEQ ID NO: 61), X-PD-C(Sx)-QY*[pY]NDF-Z (SEQ ID NO: 62), X-GAGEE-C(Sx)-DY*[pY][pY]IWAGKK-Z (SEQ ID NO: 63), and X-GEE-C(Sx)-DY*[pY][pY]IWA-Z (SEQ ID NO: 64); wherein X is H or acetyl; and Z is OH or NH2.

In some embodiments, the metal-binding compound of the present invention is selected from the group consisting of: X-EEEEY*F-C(Sx)-LV-Z (SEQ ID NO: 65), X-GADPQEY*I-C(Sx)-LPAKKKG-Z (SEQ ID NO: 66), X-GAEDEDY*E-C(Sx)-VGAKKKG-Z (SEQ ID NO: 67), X-GAEEPIY*I-C(Sx)-VPAKKKG-Z (SEQ ID NO: 42), X-GADEQIY*W-C(Sx)-IAAKKKG-Z (SEQ ID NO: 68), X-GAPE-C(Sx)-IY*ATPGAKKK-Z (SEQ ID NO: 69), and X-GAPEVIY*A-C(Sx)-PGAKKK-Z (SEQ ID NO: 70); wherein X is H or acetyl; and Z is OH or NH2.

In some embodiments, the metal-binding compound of the present invention is selected from the group consisting of: X-GAEE-C(Sx)-IY*GIFGAKKKG-Z (SEQ ID NO: 71), X-C(Sx)-DY*VEFKKK-Z (SEQ ID NO: 72), X-RRRRRRSETDDY*A-C(Sx)-IID-Z (SEQ ID NO: 73), X-RRRRRRSET-C(Sx)-DY*AEIID-Z (SEQ ID NO: 74), and X-GARAFIY*A-C(Sx)-IPAKKKG-Z (SEQ ID NO: 75); wherein X is H or acetyl; and Z is OH or NH2.

In some embodiments, the metal-binding compound of the present invention is selected from the group consisting of: X-VYS-C(Sx)-DY*YRLFNPS-Z (SEQ ID NO: 76), X-A-C(Sx)-EY*LIPQQ-Z (SEQ ID NO: 77), X-DE-C(Sx)-DY*YEIP-Z (SEQ ID NO: 78), X-PD-C(Sx)-QY*YNDF-Z (SEQ ID NO: 79), X-EGDEDGIY*V-C(Sx)-FEPKT-Z (SEQ ID NO: 80), X-EGDED-C(Sx)-IY*VNFEPKT-Z (SEQ ID NO: 81), X-PAAENPEY*L-C(Sx)-QQDPV-Z (SEQ ID NO: 82), X-PAAEN-C(Sx)-EY*LWQQDPV-Z (SEQ ID NO: 83), X-IYS-C(Sx)-DY*YR-Z (SEQ ID NO: 84), X-KGGEE-C(Sx)-EY*FELVKK-Z (SEQ ID NO: 85), X-KGGEEEEY*F-C(Sx)-LVKK-Z (SEQ ID NO: 86), X-KVEKIGE-C(Sx)-TY*GVVYK-Z (SEQ ID NO: 87), X-RRLIEDNEY*T-C(Sx)-RG-Z (SEQ ID NO: 88), X-RRLIEDAEY*A-C(Sx)-RG-Z (SEQ ID NO: 89), X-EF-C(Sx)-IY*DFLPAKKK-Z (SEQ ID NO: 90), X-EFPIY*D-C(Sx)-LPAKKK-Z (SEQ ID NO: 91), X-GEE-C(Sx)-LY*WSFPAKKK-Z (SEQ ID NO: 92), X-GEEPLY*W-C(Sx)-FPAKKK-Z (SEQ ID NO: 57), X-E-C(Sx)-LY*WSFPA-Z (SEQ ID NO: 93), X-EPLY*W-C(Sx)-FPA-Z (SEQ ID NO: 94), and X-C(Sx)-IY*GSFK-Z (SEQ ID NO: 95), X-KKKKEEIY*F-C(Sx)-FG-Z (SEQ ID NO: 96), X-KKKKEEIY*F-C(Sx)-FG-Z (SEQ ID NO: 96); wherein X is H or acetyl; and Z is OH or NH2.

In some embodiments, the metal-binding compound of the present invention is selected from the group consisting of: X-ETSK-C(Sx)-IY*DFIEK-Z (SEQ ID NO: 97), and X-ETSKVIY*D-C(Sx)-IEK-Z (SEQ ID NO: 98); wherein X is H or acetyl; and Z is OH or NH2.

In some embodiments, the metal-binding compound of the present invention is selected from the group consisting of: X-ARKRERTY*SF-C(Sx)-HH-Z (SEQ ID NO: 99), X-VP-C(Sx)-LS*PGPF-Z (SEQ ID NO: 100), X-C(Sx)-LS*PGPF-Z (SEQ ID NO: 101), X-C(Sx)-RT*PGGRR-Z (SEQ ID NO: 102), and X-C(Sx)-GT*PSGEAPNQALLR-Z (SEQ ID NO: 103); wherein X is H or acetyl; and Z is OH or NH2.

In some embodiments, the metal-binding compound of the present invention is selected from the group consisting of: X-VLTQMGSPSI-C(Sx)-SS*[pS]VS-Z (SEQ ID NO: 104), X-ESTSAPLS*P-C(Sx)-VTTSP-Z (SEQ ID NO: 105), X-GFLSRPLS*P-C(Sx)-FTTSP-Z (SEQ ID NO: 106), X-PSLLRPLS*P-C(Sx)-FFGAY-Z (SEQ ID NO: 107), X-STPSSPSS*P-C(Sx)-SSVAY-Z (SEQ ID NO: 108), X-SHGSAPLS*P-C(Sx)-APLTS-Z (SEQ ID NO: 109), X-SPFSVLSS*P-C(Sx)-SGHSN-Z (SEQ ID NO: 110), X-ALKLVRYPS*F-C(Sx)-IT-Z (SEQ ID NO: 111), X-ALKLSRYPS*F-C(Sx)-I-Z (SEQ ID NO: 112), X-RKKFGEREKT*K-C(Sx)-RIRL-Z (SEQ ID NO: 113), X-DYMR-C(Sx)-SS*RRRIRMAHQT-Z (SEQ ID NO: 114), X-DYMSAR-C(Sx)-SS*RRRIRHQT-Z (SEQ ID NO: 115), X-AHLQR-C(Sx)-LS*FRRR-Z (SEQ ID NO: 116), X-GRTGR-C(Sx)-NS*FRRR-Z (SEQ ID NO: 117), X-ALKLSR-C(Sx)-PS*FRRR-Z (SEQ ID NO: 118), X-RKRDR-C(Sx)-GT*FRRR-Z (SEQ ID NO: 119), and X-ARKRER-C(Sx)-YS*FRRR-Z (SEQ ID NO: 120); wherein X is H or acetyl; and Z is OH or NH2.

In some embodiments, the metal-binding compound of the present invention is selected from the group consisting of: X-HMRSAMS*V-C(Sx)-HLVK-Z (SEQ ID NO: 121), X-HMRSSMS*V-C(Sx)-HLVKRR-Z (SEQ ID NO: 122), X-HMRSSMS*V-C(Sx)-HLVK-Z (SEQ ID NO: 123), X-HMRSAMS*V-C(Sx)-HLV-Z (SEQ ID NO: 124), X-LRRAS*L-C(Sx)-Z (SEQ ID NO: 125), X-RRREEEEES*A-(cSx)-AA-Z (SEQ ID NO: 126), X-LSNLYHQGKFLQT*F-C(Sx)-GSPLY-Z (SEQ ID NO: 127), X-QGKF-C(Sx)-QT*FSGSPLYRRR-Z (SEQ ID NO: 128), X-KKRNRRLS*V-C(Sx)-Z (SEQ ID NO: 129), and X-AKRRRLAS*L-C(Sx)ASTSK-Z (SEQ ID NO: 130); wherein X is H or acetyl; and Z is OH or NH2.

In some embodiments, the metal-binding compound of the present invention is selected from the group consisting of: X-GRPRAAT*F-C(Sx)-EG-Z (SEQ ID NO: 131), X-GRP-MeArg-AFT*F-C(Sx)-EG-Z (SEQ ID NO: 132), X-GRP-MeArg-C(Sx)-FT*F-Sarc-EG-Z (SEQ ID NO: 133), X-RPRAAT*F-C(Sx)-Z (SEQ ID NO: 134), X-RPRAAT*F-C(Sx)-HHA-Z (SEQ ID NO: 135), X-FFKKIVTPRT*P-C(Sx)-P-Z (SEQ ID NO: 136), X-FFKNIVTPRT*P-C(Sx)-PSQGK-Z (SEQ ID NO: 137), and X-APRT*P-C(Sx)-GRR-Z (SEQ ID NO: 138); wherein X is H or acetyl; and Z is OH or NH2.

In some embodiments, the metal-binding compound of the present invention is selected from the group consisting of: X-KKKVLTQMGSPSIR-C(Sx)-SS*VS-Z (SEQ ID NO: 139), X-KKKVLTQMGSPSI-C(Sx)-SS*[pS]VS-Z (SEQ ID NO: 140), X-SGRSRRKS*P-C(Sx)-PSPHP-Z (SEQ ID NO: 141), X-SGRSR-C(Sx)-KS*PRPSPHP-Z (SEQ ID NO: 142), X-SGRSR-C(Sx)-KS*VRRSPHP-Z (SEQ ID NO: 143), X-KKLNRTLS*F-C(Sx)-EPG-Z (SEQ ID NO: 144), X-KKRPQRATS*N-C(Sx)-FAM-Z (SEQ ID NO: 145), X-RGRSRRDS*F-C(Sx)-VSPHY-Z (SEQ ID NO: 146), X-RKRSRRKS*F-C(Sx)-VLSSL-Z (SEQ ID NO: 147), X-AKRRRLSSLRASTSK-C(Sx)-ES*SQK-Z (SEQ ID NO: 148), X-AKRRRLSSLRASTSKSES*S-C(Sx)-K-Z (SEQ ID NO: 149), X-KMQPPR-C(Sx)-RS*SIMSIT-Z (SEQ ID NO: 150), X-KMQPPRSRS*S-C(Sx)-MSIT-Z (SEQ ID NO: 151), X-SGRSRPLS*P-C(Sx)-PSPHY-Z (SEQ ID NO: 152), X-LIDS*M-C(Sx)-NSFVG-Z (SEQ ID NO: 153), X-LIDSM-C(Sx)-NS*FVG-Z (SEQ ID NO: 154), X-PKRSR-C(Sx)-SS*YPNGHRD-Z (SEQ ID NO: 155), X-PKRSRSSS*V-C(Sx)-PSHSY-Z (SEQ ID NO: 156), X-SGLEDDDY*V-C(Sx)-PGGHW-Z (SEQ ID NO: 157), X-ALKLVRYPS*F-C(Sx)-ITAKKK-Z (SEQ ID NO: 158), X-AMRLERQDS*I-C(Sx)-YPKKK-Z (SEQ ID NO: 159), X-KKKVSRSGLYRSPS*M-C(Sx)-ENLNRPR-Z (SEQ ID NO: 160), X-KKVSRSGLYRSPS*M-C(Sx)-E-Z (SEQ ID NO: 161), X-KKVSRSGLYR-C(Sx)-PS*MPE-Z (SEQ ID NO: 162), X-GRPRTSS*F-C(Sx)-EPG-Z (SEQ ID NO: 163), X-LRREILS*R-C(Sx)-PSYRK-Z (SEQ ID NO: 164), X-LRREILSR-C(Sx)-PS*YRK-Z (SEQ ID NO: 165), X-LRRE-C(Sx)-LS*RRPSYRK-Z (SEQ ID NO: 166), X-LRREILSRRPS*Y-C(Sx)-K-Z (SEQ ID NO: 167), X-FQREGFGRQS*M-C(Sx)-EKR-Z (SEQ ID NO: 168), X-FQREGFG-C(Sx)-QS*MSEKR-Z (SEQ ID NO: 169), X-RS*R-C(Sx)-RSRSRSRSRSR-Z (SEQ ID NO: 170), X-RSR-C(Sx)-RS*RSRSRSRSR-Z (SEQ ID NO: 171), X-RSRSRS*R-C(Sx)-RSRSRSR-Z (SEQ ID NO: 172), X-RSRSRSR-C(Sx)-RS*RSRSR-Z (SEQ ID NO: 173), X-RSRSRSRSRS*R-C(Sx)-RSR-Z (SEQ ID NO: 174), X-RSRSRSRSRSR-C(Sx)-RS*R-Z (SEQ ID NO: 175), X-ILLSELS*R-C(Sx)-RIR-Z (SEQ ID NO: 176), X-ILLSESS*R-C(Sx)-RIR-Z (SEQ ID NO: 177), X-ILLR-C(Sx)-SS*RRIRR-Z (SEQ ID NO: 178), X-ILLS-C(Sx)-SS*RRRIRSGNKLGRIGRN-Z (SEQ ID NO: 179), X-ILLS-C(Sx)-SS*RRRIRSGNKLGRIGRN-Z (SEQ ID NO: 179), X-GKRGD-C(Sx)-KS*VSRSSSS-Z (SEQ ID NO: 180), X-SGRSR-C(Sx)-DS*PRPGTHK-Z (SEQ ID NO: 181), X-SKRSR-C(Sx)-KS*PRRSLSY-Z (SEQ ID NO: 182), X-R-C(Sx)-LS*FRRR-Z (SEQ ID NO: 183), X-PLSRT*L-C(Sx)-VAAKK-Z (SEQ ID NO: 184), X-KKKKK-C(Sx)-FS*FKKSFKLSGFSFKKNKK-Z (SEQ ID NO: 185), X-KKKKKRFS*F-C(Sx)-KSFKLSGFSFKKNKK-Z (SEQ ID NO: 186), X-KKKKKRFSF-C(Sx)-KS*FKLSGFSFKKNKK-Z (SEQ ID NO: 187), X-KKKKKRFSFKKS*F-C(Sx)-LSGFSFKKNKK-Z (SEQ ID NO: 188), X-KKKKKRFSFKKSF-C(Sx)-LS*GFSFKKNKK-Z (SEQ ID NO: 189), X-KKKKKRFSFKKSFKLSGFS*F-C(Sx)-KNKK-Z (SEQ ID NO: 190), X-KGTLVQTKGTGASGS*F-C(Sx)-LNKK-Z (SEQ ID NO: 191), X-RRR-C(Sx)-SS*LRA-Z (SEQ ID NO: 192), X-RR-C(Sx)-SS*LRA-Z (SEQ ID NO: 193), X-RRLSS*L-C(Sx)-A-Z (SEQ ID NO: 194), X-RT-C(Sx)-RS*GSVYEPLKI-Z (SEQ ID NO: 195), X-RFAVRDMRQT*V-C(Sx)-VGVIKAVDKK-Z (SEQ ID NO: 196), X-AAKI-C(Sx)-AS*FRGHMARKK-Z (SEQ ID NO: 197), X-RR-C(Sx)-RT*GRGRRGIFR-Z (SEQ ID NO: 198), X-QK-C(Sx)-PS*QRSKYL-Z (SEQ ID NO: 199), X-STASQDVANRFARKGS*L-C(Sx)-QKNV-Z (SEQ ID NO: 200), X-MFAVRDRRQT*V-C(Sx)-KGVIKAVDAV-Z (SEQ ID NO: 201), X-RLGRDK-C(Sx)-KT*LRQIRQ-Z (SEQ ID NO: 202), X-RLGRDKYKT*L-C(Sx)-QIRQ-Z (SEQ ID NO: 203), X-ERMRPRKRQGS*V-C(Sx)RRV-Z (SEQ ID NO: 204), X-HAT*P-C(Sx)-KKKRK-Z (SEQ ID NO: 205), X-GGG-C(Sx)-AT*PKKAKKL-Z (SEQ ID NO: 206), X-C(Sx)-QS*PKKG-Z (SEQ ID NO: 207), X-RRRFRPAS*P-C(Sx)-RGPPK-Z (SEQ ID NO: 208), X-RRRFRPAS*P-C(Sx)-RGPPK-Z (SEQ ID NO: 208), X-IPTS*P-C(Sx)-TITYFFF-Z (SEQ ID NO: 209), X-I-C(Sx)-TS*PITITYFFF-Z (SEQ ID NO: 210), X-IPTS*P-C(Sx)-TITYFFFKKK-Z (SEQ ID NO: 211), X-C(Sx)-KT*PKKAKKL-Z (SEQ ID NO: 212), and X-GGG-C(Sx)-AT*PKKAKKL-Z (SEQ ID NO: 206); wherein X is H or acetyl; and Z is OH or NH2.

In some embodiments, the metal-binding compound of the present invention is selected from the group consisting of: X-HMRSAMS*G-C(Sx)-HLVKRR-Z (SEQ ID NO: 213), X-HMRS-C(Sx)-MS*GLHLVKRR-Z (SEQ ID NO: 214), X-AMARAAS*A-C(Sx)-ALARRR-Z (SEQ ID NO: 215), X-KKKALSRQFS*V-C(Sx)-Z (SEQ ID NO: 216), X-GRTGRRNS*I-C(Sx)-Z (SEQ ID NO: 217), X-FLAKRRRLSS*L-C(Sx)-A-Z (SEQ ID NO: 218), X-KKRNRTLT*V-C(Sx)-Z (SEQ ID NO: 219), X-C(Sx)-FS*LRRKAK-Z (SEQ ID NO: 220), X-KEIYNT*I-C(Sx)-Z (SEQ ID NO: 221), X-RKIS*A-C(Sx)-EFDRPLR-Z (SEQ ID NO: 222), X-R-C(Sx)-IS*ASEFDRPLR-Z (SEQ ID NO: 223), X-RKIS*A-C(Sx)-EFDRPLR-Z (SEQ ID NO: 222), X-RKRS*R-C(Sx)-E-Z (SEQ ID NO: 224), X-RARTLS*F-C(Sx)-EPG-Z (SEQ ID NO: 225), X-KRRE-C(Sx)-LS*RRP[pS]YR-Z (SEQ ID NO: 226), X-RRAAEE-C(Sx)-DS*RAG[pS]PQL-Z (SEQ ID NO: 227), X-RYR-C(Sx)-PT*GMYY-Z (SEQ ID NO: 228), X-RYRHPT*R-C(Sx)-YY-Z (SEQ ID NO: 229), X-KPDRKKRY*TV-C(Sx)-GNPY-Z (SEQ ID NO: 230), X-RRRLS*F-C(Sx)-EPG-Z (SEQ ID NO: 231), X-PL-C(Sx)-RT*LSVAGLPGKK-Z (SEQ ID NO: 232), X-KKLNRT*L-C(Sx)-VA-Z (SEQ ID NO: 233), X-KKALRRQET*V-C(Sx)-AL-Z (SEQ ID NO: 234), X-LSNLYHQGKFLQT*F-C(Sx)-GSPLYRRR-Z (SEQ ID NO: 235), X-RISDELMDAT*F-C(Sx)-DQEAK-Z (SEQ ID NO: 236), X-DELMEFS*F-C(Sx)-DQEAKV-Z (SEQ ID NO: 237), X-SGEDT*L-C(Sx)-DSDDED-Z (SEQ ID NO: 238), X-GRHD[pS]GLDS*M-C(Sx)-Z (SEQ ID NO: 239), X-KRRRALS[pS]VAS*L-C(Sx)-Z (SEQ ID NO: 240), X-RRKDLHDDEEDEAMS*I-C(Sx)-A-Z (SEQ ID NO: 241), X-RRA-C(Sx)-DS*DDDDD-Z (SEQ ID NO: 242), X-LGKEF-C(Sx)-RS*YRLRYSRDGRR-Z (SEQ ID NO: 243), X-LGKEFSRS*Y-C(Sx)LRYSRDGRR-Z (SEQ ID NO: 244), X-GRPRTSS*F-C(Sx)-EG-Z (SEQ ID NO: 245), X-PRTSS*F-C(Sx)-E-Z (SEQ ID NO: 246), X-KRRRLAS*L-C(Sx)-A-Z (SEQ ID NO: 247), X-KKRNRTLS*V-C(Sx)-Z (SEQ ID NO: 248), X-RKKFGES-C(Sx)-KT*KTKEFL-Z (SEQ ID NO: 249), and X-RKKFGESEKT*K-C(Sx)-KEFL-Z (SEQ ID NO: 250); wherein X is H or acetyl; and Z is OH or NH2.

In some embodiments, the metal-binding compound of the present invention is selected from the group consisting of: X-EDPDY*F-C(Sx)-FG-Z (SEQ ID NO: 251), X-EDPDY*F-C(Sx)-FK-Z (SEQ ID NO: 252), X-EDPDY*F-C(Sx)-FP-Z (SEQ ID NO: 253), X-EDPDY*F-C(Sx)-IG-Z (SEQ ID NO: 254), X-EDPDY*F-C(Sx)-IK-Z (SEQ ID NO: 255), X-EDPDY*F-C(Sx)-IP-Z (SEQ ID NO: 256), X-EDPDY*F-C(Sx)-MG-Z (SEQ ID NO: 257), X-EDPDY*F-C(Sx)-MK-Z (SEQ ID NO: 258), X-EDPDY*F-C(Sx)-MP-Z (SEQ ID NO: 259), X-EDPDY*F-C(Sx)-VG-Z (SEQ ID NO: 260), X-EDPDY*F-C(Sx)-VK-Z (SEQ ID NO: 261), and X-EDPDY*F-C(Sx)-VP-Z (SEQ ID NO: 262); wherein X is H or acetyl; and Z is OH or NH2.

In some embodiments, the metal-binding compound of the present invention is selected from the group consisting of: X-EDPDY*I-C(Sx)-FG-Z (SEQ ID NO: 263), X-EDPDY*I-C(Sx)-FK-Z (SEQ ID NO: 264), X-EDPDY*I-C(Sx)-FP-Z (SEQ ID NO: 265), X-EDPDY*I-C(Sx)-IG-Z (SEQ ID NO: 266), X-EDPDY*I-C(Sx)-IK-Z (SEQ ID NO: 267), X-EDPDY*I-C(Sx)-IP-Z (SEQ ID NO: 268), X-EDPDY*I-C(Sx)-MG-Z (SEQ ID NO: 269), X-EDPDY*I-C(Sx)-MK-Z (SEQ ID NO: 270), X-EDPDY*I-C(Sx)-MP-Z (SEQ ID NO: 271), X-EDPDY*I-C(Sx)-VG-Z (SEQ ID NO: 272), X-EDPDY*I-C(Sx)-VK-Z (SEQ ID NO: 273), and X-EDPDY*I-C(Sx)-VP-Z (SEQ ID NO: 274); wherein X is H or acetyl; and Z is OH or NH2.

In some embodiments, the metal-binding compound of the present invention is selected from the group consisting of: X-EDPDY*V-C(Sx)-FG-Z (SEQ ID NO: 275), X-EDPDY*V-C(Sx)-FK-Z (SEQ ID NO: 276), X-EDPDY*V-C(Sx)-FP-Z (SEQ ID NO: 277), X-EDPDY*V-C(Sx)-IG-Z (SEQ ID NO: 278), X-EDPDY*V-C(Sx)-IK-Z (SEQ ID NO: 279), X-EDPDY*V-C(Sx)-IP-Z (SEQ ID NO: 280), X-EDPDY*V-C(Sx)-MG-Z (SEQ ID NO: 281), X-EDPDY*V-C(Sx)-MK-Z (SEQ ID NO: 282), X-EDPDY*V-C(Sx)-MP-Z (SEQ ID NO: 283), X-EDPDY*V-C(Sx)-VG-Z (SEQ ID NO: 284), X-EDPDY*V-C(Sx)-VK-Z (SEQ ID NO: 285), and X-EDPDY*V-C(Sx)-VP-Z (SEQ ID NO: 286); wherein X is H or acetyl; and Z is OH or NH2.

In some embodiments, the metal-binding compound of the present invention is selected from the group consisting of: X-EDPEY*F-C(Sx)-FG-Z (SEQ ID NO: 287), X-EDPEY*F-C(Sx)-FK-Z (SEQ ID NO: 288), X-EDPEY*F-C(Sx)-FP-Z (SEQ ID NO: 289), X-EDPEY*F-C(Sx)-IG-Z (SEQ ID NO: 290), X-EDPEY*F-C(Sx)-IK-Z (SEQ ID NO: 291), X-EDPEY*F-C(Sx)-IP-Z (SEQ ID NO: 292), X-EDPEY*F-C(Sx)-MG-Z (SEQ ID NO: 293), X-EDPEY*F-C(Sx)-MK-Z (SEQ ID NO: 294), X-EDPEY*F-C(Sx)-MP-Z (SEQ ID NO: 295), X-EDPEY*F-C(Sx)-VG-Z (SEQ ID NO: 296), X-EDPEY*F-C(Sx)-VK-Z (SEQ ID NO: 297), and X-EDPEY*F-C(Sx)-VP-Z (SEQ ID NO: 298); wherein X is H or acetyl; and Z is OH or NH2.

In some embodiments, the metal-binding compound of the present invention is selected from the group consisting of: X-EDPEY*I-C(Sx)-FG-Z (SEQ ID NO: 299), X-EDPEY*I-C(Sx)-FK-Z (SEQ ID NO: 300), X-EDPEY*I-C(Sx)-FP-Z (SEQ ID NO: 301), X-EDPEY*I-C(Sx)-IG-Z (SEQ ID NO: 302), X-EDPEY*I-C(Sx)-IK-Z (SEQ ID NO: 303), X-EDPEY*I-C(Sx)-IP-Z (SEQ ID NO: 304), X-EDPEY*I-C(Sx)-MG-Z (SEQ ID NO: 305), X-EDPEY*I-C(Sx)-MK-Z (SEQ ID NO: 306), X-EDPEY*I-C(Sx)-MP-Z (SEQ ID NO: 307), X-EDPEY*I-C(Sx)-VG-Z (SEQ ID NO: 308), X-EDPEY*I-C(Sx)-VK-Z (SEQ ID NO: 309), and X-EDPEY*I-C(Sx)-VP-Z (SEQ ID NO: 310); wherein X is H or acetyl; and Z is OH or NH2.

In some embodiments, the metal-binding compound of the present invention is selected from the group consisting of: X-EDPEY*V-C(Sx)-FG-Z (SEQ ID NO: 311), X-EDPEY*V-C(Sx)-FK-Z (SEQ ID NO: 312), X-EDPEY*V-C(Sx)-FP-Z (SEQ ID NO: 313), X-EDPEY*V-C(Sx)-IG-Z (SEQ ID NO: 314), X-EDPEY*V-C(Sx)-IK-Z (SEQ ID NO: 315), X-EDPEY*V-C(Sx)-IP-Z (SEQ ID NO: 316), X-EDPEY*V-C(Sx)-MG-Z (SEQ ID NO: 317), X-EDPEY*V-C(Sx)-MK-Z (SEQ ID NO: 318), X-EDPEY*V-C(Sx)-MP-Z (SEQ ID NO: 319), X-EDPEY*V-C(Sx)-VG-Z (SEQ ID NO: 320), X-EDPEY*V-C(Sx)-VK-Z (SEQ ID NO: 321), and X-EDPEY*V-C(Sx)-VP-Z (SEQ ID NO: 322); wherein X is H or acetyl; and Z is OH or NH2.

In some embodiments, the metal-binding compound of the present invention is selected from the group consisting of: X-EDPIY*F-C(Sx)-FG-Z (SEQ ID NO: 323), X-EDPIY*F-C(Sx)-FK-Z (SEQ ID NO: 324), X-EDPIY*F-C(Sx)-FP-Z (SEQ ID NO: 325), X-EDPIY*F-C(Sx)-IG-Z (SEQ ID NO: 326), X-EDPIY*F-C(Sx)-IK-Z (SEQ ID NO: 327), X-EDPIY*F-C(Sx)-IP-Z (SEQ ID NO: 328), X-EDPIY*F-C(Sx)-MG-Z (SEQ ID NO: 329), X-EDPIY*F-C(Sx)-MK-Z (SEQ ID NO: 330), X-EDPIY*F-C(Sx)-MP-Z (SEQ ID NO: 331), X-EDPIY*F-C(Sx)-VG-Z (SEQ ID NO: 332), X-EDPIY*F-C(Sx)-VK-Z (SEQ ID NO: 333), and X-EDPIY*F-C(Sx)-VP-Z (SEQ ID NO: 334); wherein X is H or acetyl; and Z is OH or NH2.

In some embodiments, the metal-binding compound of the present invention is selected from the group consisting of: X-EDPIY*I-C(Sx)-FG-Z (SEQ ID NO: 335), X-EDPIY*I-C(Sx)-FK-Z (SEQ ID NO: 336), X-EDPIY*I-C(Sx)-FP-Z (SEQ ID NO: 337), X-EDPIY*I-C(Sx)-IG-Z (SEQ ID NO: 338), X-EDPIY*I-C(Sx)-IK-Z (SEQ ID NO: 339), X-EDPIY*I-C(Sx)-IP-Z (SEQ ID NO: 340), X-EDPIY*I-C(Sx)-MG-Z (SEQ ID NO: 341), X-EDPIY*I-C(Sx)-MK-Z (SEQ ID NO: 342), X-EDPIY*I-C(Sx)-MP-Z (SEQ ID NO: 343), X-EDPIY*I-C(Sx)-VG-Z (SEQ ID NO: 344), X-EDPIY*I-C(Sx)-VK-Z (SEQ ID NO: 345), and X-EDPIY*I-C(Sx)-VP-Z (SEQ ID NO: 346); wherein X is H or acetyl; and Z is OH or NH2.

In some embodiments, the metal-binding compound of the present invention is selected from the group consisting of: X-EDPIY*V-C(Sx)-FG-Z (SEQ ID NO: 347), X-EDPIY*V-C(Sx)-FK-Z (SEQ ID NO: 348), X-EDPIY*V-C(Sx)-FP-Z (SEQ ID NO: 349), X-EDPIY*V-C(Sx)-IG-Z (SEQ ID NO: 350), X-EDPIY*V-C(Sx)-IK-Z (SEQ ID NO: 351), X-EDPIY*V-C(Sx)-IP-Z (SEQ ID NO: 352), X-EDPIY*V-C(Sx)-MG-Z (SEQ ID NO: 353), X-EDPIY*V-C(Sx)-MK-Z (SEQ ID NO: 354), X-EDPIY*V-C(Sx)-MP-Z (SEQ ID NO: 355), X-EDPIY*V-C(Sx)-VG-Z (SEQ ID NO: 356), X-EDPIY*V-C(Sx)-VK-Z (SEQ ID NO: 357), and X-EDPIY*V-C(Sx)-VP-Z (SEQ ID NO: 358); wherein X is H or acetyl; and Z is OH or NH2.

In some embodiments, the metal-binding compound of the present invention is selected from the group consisting of: X-EEPDY*F-C(Sx)-FG-Z (SEQ ID NO: 359), X-EEPDY*F-C(Sx)-FK-Z (SEQ ID NO: 360), X-EEPDY*F-C(Sx)-FP-Z (SEQ ID NO: 361), X-EEPDY*F-C(Sx)-IG-Z (SEQ ID NO: 362), X-EEPDY*F-C(Sx)-IK-Z (SEQ ID NO: 363), X-EEPDY*F-C(Sx)-IP-Z (SEQ ID NO: 364), X-EEPDY*F-C(Sx)-MG-Z (SEQ ID NO: 365), X-EEPDY*F-C(Sx)-MK-Z (SEQ ID NO: 366), X-EEPDY*F-C(Sx)-MP-Z (SEQ ID NO: 367), X-EEPDY*F-C(Sx)-VG-Z (SEQ ID NO: 368), X-EEPDY*F-C(Sx)-VK-Z (SEQ ID NO: 369), and X-EEPDY*F-C(Sx)-VP-Z (SEQ ID NO: 370); wherein X is H or acetyl; and Z is OH or NH2.

In some embodiments, the metal-binding compound of the present invention is selected from the group consisting of: X-EEPDY*I-C(Sx)-FG-Z (SEQ ID NO: 371), X-EEPDY*I-C(Sx)-FK-Z (SEQ ID NO: 372), X-EEPDY*I-C(Sx)-FP-Z (SEQ ID NO: 373), X-EEPDY*I-C(Sx)-IG-Z (SEQ ID NO: 374), X-EEPDY*I-C(Sx)-IK-Z (SEQ ID NO: 375), X-EEPDY*I-C(Sx)-IP-Z (SEQ ID NO: 376), X-EEPDY*I-C(Sx)-MG-Z (SEQ ID NO: 377), X-EEPDY*I-C(Sx)-MK-Z (SEQ ID NO: 378), X-EEPDY*I-C(Sx)-MP-Z (SEQ ID NO: 379), X-EEPDY*I-C(Sx)-VG-Z (SEQ ID NO: 380), X-EEPDY*I-C(Sx)-VK-Z (SEQ ID NO: 381), and X-EEPDY*I-C(Sx)-VP-Z (SEQ ID NO: 382); wherein X is H or acetyl; and Z is OH or NH2.

In some embodiments, the metal-binding compound of the present invention is selected from the group consisting of: X-EEPDY*V-C(Sx)-FG-Z (SEQ ID NO: 383), X-EEPDY*V-C(Sx)-FK-Z (SEQ ID NO: 384), X-EEPDY*V-C(Sx)-FP-Z (SEQ ID NO: 385), X-EEPDY*V-C(Sx)-IG-Z (SEQ ID NO: 386), X-EEPDY*V-C(Sx)-IK-Z (SEQ ID NO: 387), X-EEPDY*V-C(Sx)-IP-Z (SEQ ID NO: 388), X-EEPDY*V-C(Sx)-MG-Z (SEQ ID NO: 389), X-EEPDY*V-C(Sx)-MK-Z (SEQ ID NO: 390), X-EEPDY*V-C(Sx)-MP-Z (SEQ ID NO: 391), X-EEPDY*V-C(Sx)-VG-Z (SEQ ID NO: 392), X-EEPDY*V-C(Sx)-VK-Z (SEQ ID NO: 393), and X-EEPDY*V-C(Sx)-VP-Z (SEQ ID NO: 394); wherein X is H or acetyl; and Z is OH or NH2.

In some embodiments, the metal-binding compound of the present invention is selected from the group consisting of: X-EEPEY*F-C(Sx)-FG-Z (SEQ ID NO: 395), X-EEPEY*F-C(Sx)-FK-Z (SEQ ID NO: 396), X-EEPEY*F-C(Sx)-FP-Z (SEQ ID NO: 397), X-EEPEY*F-C(Sx)-IG-Z (SEQ ID NO: 398), X-EEPEY*F-C(Sx)-IK-Z (SEQ ID NO: 399), X-EEPEY*F-C(Sx)-IP-Z (SEQ ID NO: 400), X-EEPEY*F-C(Sx)-MG-Z (SEQ ID NO: 401), X-EEPEY*F-C(Sx)-MK-Z (SEQ ID NO: 402), X-EEPEY*F-C(Sx)-MP-Z (SEQ ID NO: 403), X-EEPEY*F-C(Sx)-VG-Z (SEQ ID NO: 404), X-EEPEY*F-C(Sx)-VK-Z (SEQ ID NO: 405), and X-EEPEY*F-C(Sx)-VP-Z (SEQ ID NO: 406); wherein X is H or acetyl; and Z is OH or NH2.

In some embodiments, the metal-binding compound of the present invention is selected from the group consisting of: X-EEPEY*I-C(Sx)-FG-Z (SEQ ID NO: 407), X-EEPEY*I-C(Sx)-FK-Z (SEQ ID NO: 408), X-EEPEY*I-C(Sx)-FP-Z (SEQ ID NO: 409), X-EEPEY*I-C(Sx)-IG-Z (SEQ ID NO: 410), X-EEPEY*I-C(Sx)-IK-Z (SEQ ID NO: 411), X-EEPEY*I-C(Sx)-IP-Z (SEQ ID NO: 412), X-EEPEY*I-C(Sx)-MG-Z (SEQ ID NO: 413), X-EEPEY*I-C(Sx)-MK-Z (SEQ ID NO: 414), X-EEPEY*I-C(Sx)-MP-Z (SEQ ID NO: 415), X-EEPEY*I-C(Sx)-VG-Z (SEQ ID NO: 416), X-EEPEY*I-C(Sx)-VK-Z (SEQ ID NO: 417), and X-EEPEY*I-C(Sx)-VP-Z (SEQ ID NO: 418); wherein X is H or acetyl; and Z is OH or NH2.

In some embodiments, the metal-binding compound of the present invention is selected from the group consisting of: X-EEPEY*V-C(Sx)-FG-Z (SEQ ID NO: 419), X-EEPEY*V-C(Sx)-FK-Z (SEQ ID NO: 420), X-EEPEY*V-C(Sx)-FP-Z (SEQ ID NO: 421), X-EEPEY*V-C(Sx)-IG-Z (SEQ ID NO: 422), X-EEPEY*V-C(Sx)-IK-Z (SEQ ID NO: 423), X-EEPEY*V-C(Sx)-IP-Z (SEQ ID NO: 424), X-EEPEY*V-C(Sx)-MG-Z (SEQ ID NO: 425), X-EEPEY*V-C(Sx)-MK-Z (SEQ ID NO: 426), X-EEPEY*V-C(Sx)-MP-Z (SEQ ID NO: 427), X-EEPEY*V-C(Sx)-VG-Z (SEQ ID NO: 428), X-EEPEY*V-C(Sx)-VK-Z (SEQ ID NO: 429), and X-EEPEY*V-C(Sx)-VP-Z (SEQ ID NO: 430); wherein X is H or acetyl; and Z is OH or NH2.

In some embodiments, the metal-binding compound of the present invention is selected from the group consisting of: X-EEPIY*A-C(Sx)-[Nle]-K-Z (SEQ ID NO: 431), X-EEPIY*A-C(Sx)-[Nle]-M-Z (SEQ ID NO: 432), X-EEPIY*A-C(Sx)-[Nle]-P-Z (SEQ ID NO: 433), X-EEPIY*A-C(Sx)-FK-Z (SEQ ID NO: 434), X-EEPIY*A-C(Sx)-FM-Z (SEQ ID NO: 435), X-EEPIY*A-C(Sx)-FP-Z (SEQ ID NO: 436), X-EEPIY*A-C(Sx)-IK-Z (SEQ ID NO: 437), X-EEPIY*A-C(Sx)-IM-Z (SEQ ID NO: 438), X-EEPIY*A-C(Sx)-IP-Z (SEQ ID NO: 439), X-EEPIY*A-C(Sx)-LK-Z (SEQ ID NO: 440), X-EEPIY*A-C(Sx)-LM-Z (SEQ ID NO: 441), X-EEPIY*A-C(Sx)-LP-Z (SEQ ID NO: 442), X-EEPIY*A-C(Sx)-MK-Z (SEQ ID NO: 443), X-EEPIY*A-C(Sx)-MM-Z (SEQ ID NO: 444), X-EEPIY*A-C(Sx)-MP-Z (SEQ ID NO: 445), X-EEPIY*A-C(Sx)-VK-Z (SEQ ID NO: 446), X-EEPIY*A-C(Sx)-VM-Z (SEQ ID NO: 447), X-EEPIY*A-C(Sx)-VP-Z (SEQ ID NO: 448), X-EEPIY*A-C(Sx)-WK-Z (SEQ ID NO: 449), X-EEPIY*A-C(Sx)-WM-Z (SEQ ID NO: 450), X-EEPIY*A-C(Sx)-WP-Z (SEQ ID NO: 451), X-EEPIY*A-C(Sx)-XK-Z (SEQ ID NO: 452), X-EEPIY*A-C(Sx)-XM-Z (SEQ ID NO: 453), and X-EEPIY*A-C(Sx)-XP-Z (SEQ ID NO: 454); wherein X is H or acetyl; and Z is OH or NH2.

In some embodiments, the metal-binding compound of the present invention is selected from the group consisting of: X-EEPIY*F-C(Sx)-[Nle]-K-Z (SEQ ID NO: 455), X-EEPIY*F-C(Sx)-[Nle]-M-Z (SEQ ID NO: 456), X-EEPIY*F-C(Sx)-[Nle]-P-Z (SEQ ID NO: 457), X-EEPIY*F-C(Sx)-FG-Z (SEQ ID NO: 458), X-EEPIY*F-C(Sx)-FK-Z (SEQ ID NO: 459), X-EEPIY*F-C(Sx)-FM-Z (SEQ ID NO: 460), X-EEPIY*F-C(Sx)-FP-Z (SEQ ID NO: 461), X-EEPIY*F-C(Sx)-IG-Z (SEQ ID NO: 462), X-EEPIY*F-C(Sx)-IK-Z (SEQ ID NO: 463), X-EEPIY*F-C(Sx)-IM-Z (SEQ ID NO: 464), X-EEPIY*F-C(Sx)-IP-Z (SEQ ID NO: 465), X-EEPIY*F-C(Sx)-LK-Z (SEQ ID NO: 466), X-EEPIY*F-C(Sx)-LM-Z (SEQ ID NO: 467), X-EEPIY*F-C(Sx)-LP-Z (SEQ ID NO: 468), X-EEPIY*F-C(Sx)-MG-Z (SEQ ID NO: 469), X-EEPIY*F-C(Sx)-MK-Z (SEQ ID NO: 470), X-EEPIY*F-C(Sx)-MM-Z (SEQ ID NO: 471), X-EEPIY*F-C(Sx)-MP-Z (SEQ ID NO: 472), X-EEPIY*F-C(Sx)-VG-Z (SEQ ID NO: 473), X-EEPIY*F-C(Sx)-VK-Z (SEQ ID NO: 474), X-EEPIY*F-C(Sx)-VM-Z (SEQ ID NO: 475), X-EEPIY*F-C(Sx)-VP-Z (SEQ ID NO: 476), X-EEPIY*F-C(Sx)-WK-Z (SEQ ID NO: 477), X-EEPIY*F-C(Sx)-WM-Z (SEQ ID NO: 478), and X-EEPIY*F-C(Sx)-WP-Z (SEQ ID NO: 479); wherein X is H or acetyl; and Z is OH or NH2.

In some embodiments, the metal-binding compound of the present invention is selected from the group consisting of: X-EEPIY*I-C(Sx)-[Nle]-K-Z (SEQ ID NO: 480), X-EEPIY*I-C(Sx)-[Nle]-M-Z (SEQ ID NO: 481), X-EEPIY*I-C(Sx)-[Nle]-P-Z (SEQ ID NO: 482), X-EEPIY*I-C(Sx)-FG-Z (SEQ ID NO: 483), X-EEPIY*I-C(Sx)-FK-Z (SEQ ID NO: 484), X-EEPIY*I-C(Sx)-FM-Z (SEQ ID NO: 485), X-EEPIY*I-C(Sx)-FP-Z (SEQ ID NO: 486), X-EEPIY*I-C(Sx)-IG-Z (SEQ ID NO: 487), X-EEPIY*I-C(Sx)-IK-Z (SEQ ID NO: 488), X-EEPIY*I-C(Sx)-IM-Z (SEQ ID NO: 489), X-EEPIY*I-C(Sx)-IP-Z (SEQ ID NO: 490), X-EEPIY*I-C(Sx)-LK-Z (SEQ ID NO: 491), X-EEPIY*I-C(Sx)-LM-Z (SEQ ID NO: 492), X-EEPIY*I-C(Sx)-LP-Z (SEQ ID NO: 493), X-EEPIY*I-C(Sx)-MG-Z (SEQ ID NO: 494), X-EEPIY*I-C(Sx)-MK-Z (SEQ ID NO: 495), X-EEPIY*I-C(Sx)-MK-Z (SEQ ID NO: 495), X-EEPIY*I-C(Sx)-MM-Z (SEQ ID NO: 496), X-EEPIY*I-C(Sx)-MP-Z (SEQ ID NO: 497), X-EEPIY*I-C(Sx)-VG-Z (SEQ ID NO: 498), X-EEPIY*I-C(Sx)-VK-Z (SEQ ID NO: 499), X-EEPIY*I-C(Sx)-VM-Z (SEQ ID NO: 500), X-EEPIY*I-C(Sx)-WK-Z (SEQ ID NO: 501), X-EEPIY*I-C(Sx)-WM-Z (SEQ ID NO: 502), X-EEPIY*I-C(Sx)-WP-Z (SEQ ID NO: 503), X-EEPIY*I-C(Sx)-XK-Z (SEQ ID NO: 504), X-EEPIY*I-C(Sx)-XM-Z (SEQ ID NO: 505), and X-EEPIY*I-C(Sx)-XP-Z (SEQ ID NO: 506); wherein X is H or acetyl; and Z is OH or NH2.

In some embodiments, the metal-binding compound of the present invention is selected from the group consisting of: X-EEPIY*M-C(Sx)-[Nle]-K-Z (SEQ ID NO: 507), X-EEPIY*M-C(Sx)-[Nle]-M-Z (SEQ ID NO: 508), X-EEPIY*M-C(Sx)-[Nle]-P-Z (SEQ ID NO: 509), X-EEPIY*M-C(Sx)-FK-Z (SEQ ID NO: 510), X-EEPIY*M-C(Sx)-FM-Z (SEQ ID NO: 511), X-EEPIY*M-C(Sx)-FP-Z (SEQ ID NO: 512), X-EEPIY*M-C(Sx)-IK-Z (SEQ ID NO: 513), X-EEPIY*M-C(Sx)-IM-Z (SEQ ID NO: 514), X-EEPIY*M-C(Sx)-IP-Z (SEQ ID NO: 515), X-EEPIY*M-C(Sx)-LK-Z (SEQ ID NO: 516), X-EEPIY*M-C(Sx)-LM-Z (SEQ ID NO: 517), X-EEPIY*M-C(Sx)-LP-Z (SEQ ID NO: 518), X-EEPIY*M-C(Sx)-MK-Z (SEQ ID NO: 519), X-EEPIY*M-C(Sx)-MM-Z (SEQ ID NO: 520), X-EEPIY*M-C(Sx)-MP-Z (SEQ ID NO: 521), X-EEPIY*M-C(Sx)-VK-Z (SEQ ID NO: 522), X-EEPIY*M-C(Sx)-VM-Z (SEQ ID NO: 523), X-EEPIY*M-C(Sx)-VP-Z (SEQ ID NO: 524), X-EEPIY*M-C(Sx)-WK-Z (SEQ ID NO: 525), X-EEPIY*M-C(Sx)-WM-Z (SEQ ID NO: 526), and X-EEPIY*M-C(Sx)-WP-Z (SEQ ID NO: 527); wherein X is H or acetyl; and Z is OH or NH2.

In some embodiments, the metal-binding compound of the present invention is selected from the group consisting of: X-EEPIY*V-C(Sx)-[Nle]-K-Z (SEQ ID NO: 528), X-EEPIY*V-C(Sx)-[Nle]-M-Z (SEQ ID NO: 529), X-EEPIY*V-C(Sx)-[Nle]-P-Z (SEQ ID NO: 530), X-EEPIY*V-C(Sx)-FG-Z (SEQ ID NO: 531), X-EEPIY*V-C(Sx)-FK-Z (SEQ ID NO: 532), X-EEPIY*V-C(Sx)-FM-Z (SEQ ID NO: 533), X-EEPIY*V-C(Sx)-FP-Z (SEQ ID NO: 534), X-EEPIY*V-C(Sx)-IG-Z (SEQ ID NO: 535), X-EEPIY*V-C(Sx)-IK-Z (SEQ ID NO: 536), X-EEPIY*V-C(Sx)-IM-Z (SEQ ID NO: 537), X-EEPIY*V-C(Sx)-IP-Z (SEQ ID NO: 538), X-EEPIY*V-C(Sx)-LK-Z (SEQ ID NO: 539), X-EEPIY*V-C(Sx)-LM-Z (SEQ ID NO: 540), X-EEPIY*V-C(Sx)-LP-Z (SEQ ID NO: 541), X-EEPIY*V-C(Sx)-MG-Z (SEQ ID NO: 542), X-EEPIY*V-C(Sx)-MK-Z (SEQ ID NO: 543), X-EEPIY*V-C(Sx)-MM-Z (SEQ ID NO: 544), X-EEPIY*V-C(Sx)-MP-Z (SEQ ID NO: 545), X-EEPIY*V-C(Sx)-VG-Z (SEQ ID NO: 546), X-EEPIY*V-C(Sx)-VK-Z (SEQ ID NO: 547), X-EEPIY*V-C(Sx)-VM-Z (SEQ ID NO: 548), X-EEPIY*V-C(Sx)-VP-Z (SEQ ID NO: 549), X-EEPIY*V-C(Sx)-WK-Z (SEQ ID NO: 550), X-EEPIY*V-C(Sx)-WM-Z (SEQ ID NO: 551), X-EEPIY*V-C(Sx)-WP-Z (SEQ ID NO: 552), X-EEPIY*V-C(Sx)-XK-Z (SEQ ID NO: 553), X-EEPIY*V-C(Sx)-XM-Z (SEQ ID NO: 554), and X-EEPIY*V-C(Sx)-XP-Z (SEQ ID NO: 555); wherein X is H or acetyl; and Z is OH or NH2.

In some embodiments, the metal-binding compound of the present invention is selected from the group consisting of: X-EEPIY*W-C(Sx)-[Nle]-K-Z (SEQ ID NO: 556), X-EEPIY*W-C(Sx)-[Nle]-M-Z (SEQ ID NO: 557), X-EEPIY*W-C(Sx)-[Nle]-P-Z (SEQ ID NO: 558), X-EEPIY*W-C(Sx)-FK-Z (SEQ ID NO: 559), X-EEPIY*W-C(Sx)-FM-Z (SEQ ID NO: 560), X-EEPIY*W-C(Sx)-FP-Z (SEQ ID NO: 561), X-EEPIY*W-C(Sx)-IK-Z (SEQ ID NO: 562), X-EEPIY*W-C(Sx)-IM-Z (SEQ ID NO: 563), X-EEPIY*W-C(Sx)-IP-Z (SEQ ID NO: 564), X-EEPIY*W-C(Sx)-LK-Z (SEQ ID NO: 565), X-EEPIY*W-C(Sx)-LM-Z (SEQ ID NO: 566), X-EEPIY*W-C(Sx)-LP-Z (SEQ ID NO: 567), X-EEPIY*W-C(Sx)-MK-Z (SEQ ID NO: 568), X-EEPIY*W-C(Sx)-MM-Z (SEQ ID NO: 569), X-EEPIY*W-C(Sx)-MP-Z (SEQ ID NO: 570), X-EEPIY*W-C(Sx)-VK-Z (SEQ ID NO: 571), X-EEPIY*W-C(Sx)-VM-Z (SEQ ID NO: 572), X-EEPIY*W-C(Sx)-VP-Z (SEQ ID NO: 573), X-EEPIY*W-C(Sx)-WK-Z (SEQ ID NO: 574), X-EEPIY*W-C(Sx)-WM-Z (SEQ ID NO: 575), and X-EEPIY*W-C(Sx)-WP-Z (SEQ ID NO: 576); wherein X is H or acetyl; and Z is OH or NH2.

In some embodiments, the metal-binding compound of the present invention is selected from the group consisting of: X-RARRRLS*F-C(Sx)-GFGSR-Z (SEQ ID NO: 577), X-LKKLRRRLS*D-C(Sx)-VA-Z (SEQ ID NO: 578), X-SPS-C(Sx)-QS*SMVARTQTVR-Z (SEQ ID NO: 579), X-RKRDRLGT*L-C(Sx)-I-Z (SEQ ID NO: 580), X-KRRRLAS*L-C(Sx)-GHMAR-Z (SEQ ID NO: 581), X-ARARPRTYT*F-C(Sx)-HH-Z (SEQ ID NO: 582), X-KKRNRTLS*V-C(Sx)-VG-Z (SEQ ID NO: 583), X-KKRPQRRYS*N-C(Sx)-FS-Z (SEQ ID NO: 584), X-ARKRERTYT*F-C(Sx)-HH-Z (SEQ ID NO: 585), X-ARKRERAYS*F-C(Sx)-HH-Z (SEQ ID NO: 586), X-KKKRFS*F-C(Sx)-KSFKLSGFSFKK-Z (SEQ ID NO: 587), X-KKKRFSF-C(Sx)-KS*FKLSGFSFKK-Z (SEQ ID NO: 588), X-MFAVRDRRQT*V-C(Sx)-KGVIKAVD-Z (SEQ ID NO: 589), X-KGTLVQTKGTGASGS*F-C(Sx)-LNK-Z (SEQ ID NO: 590), X-KKKRFSFKKS*F-C(Sx)-LSGFSFKK-Z (SEQ ID NO: 591), X-KKKRFSFKKSF-C(Sx)-LS*GFSFKK-Z (SEQ ID NO: 592), X-RFAVRDMRQT*V-C(Sx)-VGVIKAVD-Z (SEQ ID NO: 593), X-KKK-C(Sx)-FS*FKKSFKLSGFSFKK-Z (SEQ ID NO: 594), X-KKRFSFKKSFKLSGFS*F-C(Sx)-KN-Z (SEQ ID NO: 595), X-TASQDVANRFARKGS*L-C(Sx)-QKN-Z (SEQ ID NO: 596), X-RFARKGS*L-C(Sx)-QKN-Z (SEQ ID NO: 597); wherein X is H or acetyl; and Z is OH or NH2.

In some embodiments, the metal-binding compound of the present invention is selected from the group consisting of: X-KEVPR-C(Sx)-KS*LVGTPYWMAPE-Z (SEQ ID NO: 598), X-KEVPR-C(Sx)-KS*LVGTPYWMAPE-Z (SEQ ID NO: 598); wherein X is H or acetyl; and Z is OH or NH2.

In some embodiments, the metal-binding compound of the present invention is selected from the group consisting of: X-KKALRRQET*V-C(Sx)-AL-Z (SEQ ID NO: 234), X-KLNRVFS*V-C(Sx)-VA-Z (SEQ ID NO: 599), X-TRPRKRQGS*F-C(Sx)-RR-Z (SEQ ID NO: 600), X-KKRFSFKKT*F-C(Sx)-LSGFSFKK-Z (SEQ ID NO: 601), X-ARKRERAYS*F-C(Sx)-VG-Z (SEQ ID NO: 602), X-ARKKWKQS*V-C(Sx)-LIS-Z (SEQ ID NO: 603), X-DNKLRRYT*T-C(Sx)-SKRKT-Z (SEQ ID NO: 604), X-EPPLSQET*F-C(Sx)-DLWKL-Z (SEQ ID NO: 605), X-IIMDSSIS*K-C(Sx)-ALSEI-Z (SEQ ID NO: 606), X-KTIERRYS*D-C(Sx)-TTLVA-Z (SEQ ID NO: 607), X-LSRRL-C(Sx)-VT*GDLFDIM-Z (SEQ ID NO: 608), X-NKLRRQHS*Y-C(Sx)-TFVDL-Z (SEQ ID NO: 609), X-RREER-C(Sx)-LS*APGNLLT-Z (SEQ ID NO: 610), X-KRPQRATS*N-C(Sx)-FAMFD-Z (SEQ ID NO: 611), X-VRRRWKLS*F-C(Sx)-IVSL-Z (SEQ ID NO: 612), MSSKRTKT*K-C(Sx)-KKRPQ-Z (SEQ ID NO: 613), X-ARQSRRST*Q-C(Sx)-VTLTD-Z (SEQ ID NO: 614), X-DKKGNFNY*I-C(Sx)-FTRIL-Z (SEQ ID NO: 615), X-EFKNIFGT*P-C(Sx)-FVAPE-Z (SEQ ID NO: 616), X-EYTIKSHS*S-C(Sx)-PPNNS-Z (SEQ ID NO: 617), X-KDPKRRMT*I-C(Sx)-QSLEH-Z (SEQ ID NO: 618), X-KKRPQRAT*S-C(Sx)-VFAMF-Z (SEQ ID NO: 619), X-KQTAR-C(Sx)-ST*GGKAPRK-Z (SEQ ID NO: 620), X-LGETKQET*L-C(Sx)-NISAV-Z (SEQ ID NO: 621), X-PERRRLKT*T-C(Sx)-LKEYT-Z (SEQ ID NO: 622), X-PYHFRAPS*R-C(Sx)-FWRTV-Z (SEQ ID NO: 623), X-QGRKRRQT*S-C(Sx)-TDFYH-Z (SEQ ID NO: 624), X-RPREKRRS*T-C(Sx)-VSFWT-Z (SEQ ID NO: 625), X-RQRKRHKS*D-C(Sx)-ISLSF-Z (SEQ ID NO: 626), X-SSRRR-C(Sx)-IS*ETEENSD-Z (SEQ ID NO: 627), X-TIGRSRST*R-C(Sx)-REQEN-Z (SEQ ID NO: 628), X-TEMGDRFT*D-C(Sx)-EVDEL-Z (SEQ ID NO: 629), X-TTRLK-C(Sx)-YT*IKSHSSL-Z (SEQ ID NO: 630), X-PLSQETFS*D-C(Sx)-WKLLP-Z (SEQ ID NO: 631), X-ENIPEDSS*M-C(Sx)-SKRFR-Z (SEQ ID NO: 632), X-PEDSSMVS*K-C(Sx)-FRFDD-Z (SEQ ID NO: 633), X-RFDCR-C(Sx)-IS*GLLTITP-Z (SEQ ID NO: 634), X-RRRFD-C(Sx)-RS*ISGLLTT-Z (SEQ ID NO: 635), X-SEDKTSKS*S-C(Sx)-NGT-Z (SEQ ID NO: 636), X-SKSS-C(Sx)-NGT*CGDREDK-Z (SEQ ID NO: 637), X-KKRAARASTS*N-C(Sx)-RR-Z (SEQ ID NO: 638), X-ILSR-C(Sx)-PS*YRKILN-Z (SEQ ID NO: 639), X-PLRRTLS*V-C(Sx)-ASPS-Z (SEQ ID NO: 640); wherein X is H or acetyl; and Z is OH or NH2.

In some embodiments, the metal-binding compound of the present invention is selected from the group consisting of: X-HAAIGDDDDAYS*I-C(Sx)-A-Z (SEQ ID NO: 641); wherein X is H or acetyl; and Z is OH or NH2.

In some embodiments, the metal-binding compound of the present invention is selected from the group consisting of: X-KK-C(Sx)-RLS*PGRRRK-Z (SEQ ID NO: 642), X-QGKFLQT*F-C(Sx)-GSPLYRRR-Z (SEQ ID NO: 643), X-GRDK-C(Sx)-KS*LRQIRQ-Z (SEQ ID NO: 644), X-LSR-C(Sx)-SS*PHQ[pS]EDEEE-Z (SEQ ID NO: 645), X-SR-C(Sx)-SS*PHQ[pS]EDEEEPRD-Z (SEQ ID NO: 646), X-GT*F-C(Sx)-AAIRRLAARRR-Z (SEQ ID NO: 647), X-KTGPLS*P-C(Sx)-PFK-Z (SEQ ID NO: 648), X-GLYRS*P-C(Sx)-MPENLNRPRL-Z (SEQ ID NO: 649), X-KRELVE-C(Sx)-LT*PSGEAPNQALLR-Z (SEQ ID NO: 650); wherein X is H or acetyl; and Z is OH or NH2.

In some embodiments, the metal-binding compound of the present invention is selected from the group consisting of: X-GDQDYLS*L-C(Sx)-VG-Z (SEQ ID NO: 651), X-ILSRRPS*Y-C(Sx)-KILN-Z (SEQ ID NO: 652), X-QLIDSMANS*F-C(Sx)-GTRRR-Z (SEQ ID NO: 653), X-KKERLLDDRHDS*G-C(Sx)-DSMDEE-Z (SEQ ID NO: 654), X-KKERLLDDRHDSGLDS*M-C(Sx)-EE-Z (SEQ ID NO: 655); wherein X is H or acetyl; and Z is OH or NH2.

In some embodiments, the metal-binding compound of the present invention is selected from the group consisting of: X-QDLDE-C(Sx)-VS*QEDVPLV-Z (SEQ ID NO: 656), X-ISAQIGAT*L-C(Sx)-RRLSF-Z (SEQ ID NO: 657), X-WHKTTQMS*A-C(Sx)-GTYA-Z (SEQ ID NO: 658), X-SGRPRTTS*F-C(Sx)-ESCKP-Z (SEQ ID NO: 659), X-RSGL-C(Sx)-RS*PSMPENLNRPRLK-Z (SEQ ID NO: 660), X-KDQEAKVSRSGLYRS*P-C(Sx)-MPEN-Z (SEQ ID NO: 661), X-DE-C(Sx)-EGT*FRSSIRRLSSRRR-Z (SEQ ID NO: 662), X-DEE-C(Sx)-GT*FRSSIRRLSSRRR-Z (SEQ ID NO: 663), X-DEEEGT*F-C(Sx)-SSIRRLSSRRR-Z (SEQ ID NO: 664), X-DE-C(Sx)-EGT*FRAAIRRLAARRR-Z (SEQ ID NO: 665), X-DEE-C(Sx)-GT*FRAAIRRLAARRR-Z (SEQ ID NO: 666), X-DEEEGT*F-C(Sx)-AAIRRLAARRR-Z (SEQ ID NO: 667), X-QGKF-C(Sx)-QT*FCGSPLYRRR-Z (SEQ ID NO: 668), X-HVNATY*V-C(Sx)-VKCVAP-Z (SEQ ID NO: 669), X-CKRRRLAS*L-C(Sx)-Z (SEQ ID NO: 670), X-NTFIG-C(Sx)-PY*WMAPEVI-Z (SEQ ID NO: 671), X-RRNTFIGT*P-C(Sx)-WMAPE-Z (SEQ ID NO: 672), X-RTVGRRNT*F-C(Sx)-GTPYW-Z (SEQ ID NO: 673); wherein X is H or acetyl; and Z is OH or NH2.

In some embodiments, the metal-binding compound of the present invention is selected from the group consisting of: X-GRDK-C(Sx)-KT*LRQIRQ-Z (SEQ ID NO: 674), X-QLIDS*M-C(Sx)-NSFVGTRRR-Z (SEQ ID NO: 675), X-QLIDSM-C(Sx)-NS*FVGTRRR-Z (SEQ ID NO: 676), X-RAL-C(Sx)-DS*FRRRDTAQ-Z (SEQ ID NO: 677), X-KLRALTDS*F-C(Sx)-RR-Z (SEQ ID NO: 678); wherein X is H or acetyl; and Z is OH or NH2.

In some embodiments, the metal-binding compound of the present invention is selected from the group consisting of: X-SFLSRSKS*F-C(Sx)-PLLHY-Z (SEQ ID NO: 679), X-SFLSR-C(Sx)-KS*FSPLLHY-Z (SEQ ID NO: 680), X-SFLSRSKS*F-C(Sx)-PLLHL-Z (SEQ ID NO: 681), X-SFLSR-C(Sx)-KS*FRPLLHL-Z (SEQ ID NO: 682), X-SGLSRSKS*F-C(Sx)-RLSHQ-Z (SEQ ID NO: 683), X-SGLSR-C(Sx)-KS*FRRLSHQ-Z (SEQ ID NO: 684), X-QHLRLSTS*S-C(Sx)-RLLYA-Z (SEQ ID NO: 685), X-QHLRL-C(Sx)-TS*SGRLLYA-Z (SEQ ID NO: 686), X-PLSTWSGS*P-C(Sx)-YAAPE-Z (SEQ ID NO: 687), X-PLST-C(Sx)-SGS*PPYAAPE-Z (SEQ ID NO: 688), X-PLSTW-C(Sx)-GS*PPYAAPE-Z (SEQ ID NO: 689), X-PESFSSGS*Q-C(Sx)-VHQKR-Z (SEQ ID NO: 690), X-PESF-C(Sx)-SGS*QDVHQKR-Z (SEQ ID NO: 691), X-LLATWSGS*P-C(Sx)-YAAPE-Z (SEQ ID NO: 692), X-SALNRTSS*D-C(Sx)-ALHTS-Z (SEQ ID NO: 693), X-SALNR-C(Sx)-SS*DSALHTS-Z (SEQ ID NO: 694), X-AMARAAS*A-C(Sx)-ALARRR-Z (SEQ ID NO: 215), X-AMAR-C(Sx)-AS*AAALARRR-Z (SEQ ID NO: 695), X-ALNRTSS*D-C(Sx)-ALHRRR-Z (SEQ ID NO: 696), X-ALNR-C(Sx)-SS*DSALHRRR-Z (SEQ ID NO: 697); wherein X is H or acetyl; and Z is OH or NH2.

In some embodiments, the metal-binding compound of the present invention is selected from the group consisting of: X-PKRSRSSS*F-C(Sx)-PGTRK-Z (SEQ ID NO: 698), X-PKRSR-C(Sx)-SS*FPPGTRK-Z (SEQ ID NO: 699), X-APNVH-C(Sx)-NT*IEPVNID-Z (SEQ ID NO: 700), X-PKALQRLS*R-C(Sx)-IVRSR-Z (SEQ ID NO: 701), X-ALQRLSRS*I-C(Sx)-RSRAH-Z (SEQ ID NO: 702), X-LSRSIVRS*R-C(Sx)-HSTAV-Z (SEQ ID NO: 703), X-IVRSRAHS*T-C(Sx)-VGIFS-Z (SEQ ID NO: 704), X-EIRSR-C(Sx)-SS*YPAGTED-Z (SEQ ID NO: 705), X-ARQSRRST*Q-C(Sx)-VTLTD-Z (SEQ ID NO: 614), X-SQRQRSTS*T-C(Sx)-NVH[Nle]V-Z (SEQ ID NO: 706), X-PKINRSAS*E-C(Sx)-SLHRA-Z (SEQ ID NO: 707), X-PKINR-C(Sx)-AS*EPSLHRA-Z (SEQ ID NO: 708), X-RKSGKRQT*E-C(Sx)-EKKKK-Z (SEQ ID NO: 709), X-RPRGQRDS*S-C(Sx)-YWEIE-Z (SEQ ID NO: 710), X-RPRGQ-C(Sx)-DS*SYYWEIE-Z (SEQ ID NO: 711), X-VSGQLIDS*[Nle]-C(Sx)-NSFVG-Z (SEQ ID NO: 712), X-VSGQL-C(Sx)-DS*[Nle]ANSFVG-Z (SEQ ID NO: 713), X-LIDS[Nle]ANS*F-C(Sx)-GTRSY-Z (SEQ ID NO: 714), X-LIDS[Nle]-C(Sx)-NS*FVGTRSY-Z (SEQ ID NO: 715), X-SPRSDRDS*F-C(Sx)-PLSSL-Z (SEQ ID NO: 716), X-SPRSD-C(Sx)-DS*FRPLSSL-Z (SEQ ID NO: 717), X-NVH[Nle]VSTT*L-C(Sx)-VDSR-Z (SEQ ID NO: 718), X-NVH[Nle]V-C(Sx)-TT*LPVDSR-Z (SEQ ID NO: 719), X-RRRSDDDS*P-C(Sx)-PSTHK-Z (SEQ ID NO: 720), X-RRRSD-C(Sx)-DS*PRPSTHK-Z (SEQ ID NO: 721), X-QNRSGAMS*P-C(Sx)-SWNSD-Z (SEQ ID NO: 722), X-AKELDQGS*L-C(Sx)-TSFVG-Z (SEQ ID NO: 723), X-AGTSF[Nle][Nle]T*P-C(Sx)-VVTRY-Z (SEQ ID NO: 724), X-AGTSF-C(Sx)-[Nle]T*PYVVTRY-Z (SEQ ID NO: 725), X-AGTSF-C(Sx)-[Nle]T*P[pY]VVTRY-Z (SEQ ID NO: 726), X-TSF[Nle][Nle]TPY*V-C(Sx)-TRYYR-Z (SEQ ID NO: 727), X-TSF[Nle][Nle][pT]PY*V-C(Sx)-TRYYR-Z (SEQ ID NO: 728), X-ETPPRPRT*P-C(Sx)-RPLSS-Z (SEQ ID NO: 729), X-ARLASKGT*GA-C(Sx)-EFQG-Z (SEQ ID NO: 730), X-FQGQLLGT*I-C(Sx)-F[Nle]APE-Z (SEQ ID NO: 731), X-NTIDLP[Nle]S*P-C(Sx)-TLDSL-Z (SEQ ID NO: 732), X-DPGSA-C(Sx)-PY*LKTKFI-Z (SEQ ID NO: 733), X-ISGQLVDS*I-C(Sx)-KTRDA-Z (SEQ ID NO: 734), X-ISGQL-C(Sx)-DS*IAKTRDA-Z (SEQ ID NO: 735), X-ISGRLVDS*K-C(Sx)-KTRSA-Z (SEQ ID NO: 736), X-ISGRL-C(Sx)-DS*KAKTRSA-Z (SEQ ID NO: 737), X-ISGYLVDS*V-C(Sx)-KTIDA-Z (SEQ ID NO: 738), X-ISGYL-C(Sx)-DS*VAKTIDA-Z (SEQ ID NO: 739), X-LVDSIAKT*R-C(Sx)-AGSRP-Z (SEQ ID NO: 740), X-LVDSI-C(Sx)-KT*RDAGSRP-Z (SEQ ID NO: 741), X-LVDSKAKT*R-C(Sx)-AGSAA-Z (SEQ ID NO: 742), X-LVDSK-C(Sx)-KT*RSAGSAA-Z (SEQ ID NO: 743), X-LVDSVAKT*I-C(Sx)-AGSKP-Z (SEQ ID NO: 744), X-LVDSV-C(Sx)-KT*IDAGSKP-Z (SEQ ID NO: 745), X-SQKLLDAS*K-C(Sx)-AKNFV-Z (SEQ ID NO: 746), X-YEHQRVYT*Y-C(Sx)-QSRFY-Z (SEQ ID NO: 747), X-WHKITKMS*A-C(Sx)-GTYAW-Z (SEQ ID NO: 748), X-LAREW-C(Sx)-KT*TK[Nle]SAAG-Z (SEQ ID NO: 749), X-TKQTARKS*T-C(Sx)-GKAPR-Z (SEQ ID NO: 750), X-KQTAR-C(Sx)-ST*GGKAPRK-Z (SEQ ID NO: 620), X-KRPQRATS*N-C(Sx)-FA[Nle]FD-Z (SEQ ID NO: 751), X-KKRPQRAT*S-C(Sx)-VFA[Nle]F-Z (SEQ ID NO: 752), X-TVGNKLDT*F-C(Sx)-GSPPY-Z (SEQ ID NO: 753), X-TFGNKLDT*F-C(Sx)-GSPPY-Z (SEQ ID NO: 754), X-VSTQLVNS*I-C(Sx)-KTYVG-Z (SEQ ID NO: 755), X-LVNSIAKT*Y-C(Sx)-GTNAY-Z (SEQ ID NO: 756), X-S[Nle]DGLFGT*I-C(Sx)-YLPPE-Z (SEQ ID NO: 757), X-PDKQFLIS*P-C(Sx)-ASPPV-Z (SEQ ID NO: 758), X-FLISPPAS*P-C(Sx)-VGWKQ-Z (SEQ ID NO: 759), X-LVDSVAKT*[Nle]-C(Sx)-AGSKP-Z (SEQ ID NO: 760), X-KLKNN-C(Sx)-QT*[Nle]PGEVNS-Z (SEQ ID NO: 761), X-DENFE-C(Sx)-HS*APPSPEE-Z (SEQ ID NO: 762), X-NEYLR-C(Sx)-IS*LPVPVLV-Z (SEQ ID NO: 763), X-GDNVLINT*Y-C(Sx)-GVLKI-Z (SEQ ID NO: 764), X-GINPSTET*F-C(Sx)-GTLQY-Z (SEQ ID NO: 765), X-TETFTGT*L-C(Sx)-Y[Nle]APE-Z (SEQ ID NO: 766), X-IPERDSRY*S-C(Sx)-PLHEE-Z (SEQ ID NO: 767), X-DNVLINTY*S-C(Sx)-VLKIS-Z (SEQ ID NO: 768), X-GGRRPGTS*P-C(Sx)-LLQGT-Z (SEQ ID NO: 769), X-LVQGI-C(Sx)-FS*QPTSPDH-Z (SEQ ID NO: 770), X-EDQPR-C(Sx)-QS*LDSALLE-Z (SEQ ID NO: 771), X-FPKDLRGT*E-C(Sx)-Y[Nle]SPE-Z (SEQ ID NO: 772), X-LELSRRRS*L-C(Sx)-ELHKR-Z (SEQ ID NO: 773), X-YDGERKKT*L-C(Sx)-GTPNY-Z (SEQ ID NO: 774), X-RTPGRPLS*S-C(Sx)-G[Nle]DSR-Z (SEQ ID NO: 775), X-PGRPL-C(Sx)-SY*G[Nle]DSRPP-Z (SEQ ID NO: 776), X-DGR[Nle]VQLS*P-C(Sx)-ALAAP-Z (SEQ ID NO: 777), X-SYTPTPRS*P-C(Sx)-FIGRR-Z (SEQ ID NO: 778), X-HDHTGFLT*E-C(Sx)-VATRW-Z (SEQ ID NO: 779), X-HDHTG-C(Sx)-LT*EYVATRW-Z (SEQ ID NO: 780), X-HDHTG-C(Sx)-LT*E[pY]VATRW-Z (SEQ ID NO: 781), X-HTGFLTEY*V-C(Sx)-TRWYR-Z (SEQ ID NO: 782), X-HTGFL[pT]EY*V-C(Sx)-TRWYR-Z (SEQ ID NO: 783), X-RGEPNVSY*I-C(Sx)-SRYYR-Z (SEQ ID NO: 784), X-TVNQSLLS*P-C(Sx)-VLEVD-Z (SEQ ID NO: 785), X-SPLNITST*P-C(Sx)-PDASS-Z (SEQ ID NO: 786); wherein X is H or acetyl; and Z is OH or NH2.

In some embodiments, the metal-binding compound of the present invention is selected from the group consisting of: X-AAFRLFKY*GV-C(Sx)-LYKN-Z (SEQ ID NO: 787), X-AAFRL-C(Sx)-KY*GVQLYKN-Z (SEQ ID NO: 788), X-SIESDIY*A-C(Sx)-IPDET-Z (SEQ ID NO: 789), X-DSQQTNDY*[Nle]-C(Sx)-PEEDW-Z (SEQ ID NO: 790), X-DVYEEDSY*V-C(Sx)-RSQGR-Z (SEQ ID NO: 791), X-EELA[Nle]DVY*D-C(Sx)-VDRRE-Z (SEQ ID NO: 792), X-EGGW[Nle]EDY*D-C(Sx)-VHLQG-Z (SEQ ID NO: 793), X-EIYDRREY*S-C(Sx)-FEKEQ-Z (SEQ ID NO: 794), X-ELFDDPSY*V-C(Sx)-VQNLD-Z (SEQ ID NO: 795), X-ERSW[Nle]DDY*D-C(Sx)-VHLQG-Z (SEQ ID NO: 796), X-FGIED-C(Sx)-DY*VEPSGFT-Z (SEQ ID NO: 797), X-FGLGSRAY*P-C(Sx)-FAA-Z (SEQ ID NO: 798), X-FLSEETPY*S-C(Sx)-PTGNH-Z (SEQ ID NO: 799), X-FLSEE-C(Sx)-PY*SYPTGNH-Z (SEQ ID NO: 800), X-FRKNFAKY*E-C(Sx)-FKSSP-Z (SEQ ID NO: 801), X-GW[Nle]EDYDY*V-C(Sx)-LQGKE-Z (SEQ ID NO: 802), X-IIDEEDTY*T-C(Sx)-PSTRD-Z (SEQ ID NO: 803), X-IIDEE-C(Sx)-TY*T[Nle]PSTRD-Z (SEQ ID NO: 804), X-KDNDSHVY*A-C(Sx)-IEDT-Z (SEQ ID NO: 805), X-LDGEVTPY*A-C(Sx)-TNNNA-Z (SEQ ID NO: 806), X-LPQDKEYY*K-C(Sx)-KEPGE-Z (SEQ ID NO: 807), X-NSTPESDY*D-C(Sx)-TPND[Nle]-Z (SEQ ID NO: 808), X-PAPLTPAY*H-C(Sx)-LPHPS-Z (SEQ ID NO: 809), X-PSPATDLY*Q-C(Sx)-PPGPG-Z (SEQ ID NO: 810), X-QDNDQPDY*D-C(Sx)-VASDE-Z (SEQ ID NO: 811), X-QPSSKAL*YD-C(Sx)-EPEND-Z (SEQ ID NO: 812), X-RDKQRLSY*GA-C(Sx)-TNQI-Z (SEQ ID NO: 813), X-RDKQR-C(Sx)-SY*GAFTNQI-Z (SEQ ID NO: 814), X-RETSKVIY*D-C(Sx)-IEKTG-Z (SEQ ID NO: 815), X-RLIEDNEY*T-C(Sx)-RQGAK-Z (SEQ ID NO: 816), X-RLIED-C(Sx)-EY*TARQGAK-Z (SEQ ID NO: 817), X-RY[Nle]ED-C(Sx)-TY*YKASKGK-Z (SEQ ID NO: 818), X-SGDKDHLY*S-C(Sx)-VAKPR-Z (SEQ ID NO: 819), X-SIREDDDY*D-C(Sx)-IIDHG-Z (SEQ ID NO: 820), X-SVSETDDY*A-C(Sx)-IIDEE-Z (SEQ ID NO: 821), X-VDPDNEAY*E-C(Sx)-PSEEG-Z (SEQ ID NO: 822), X-VGEEEHVY*S-C(Sx)-PNKQK-Z (SEQ ID NO: 823), X-VGEEE-C(Sx)-VY*SFPNKQK-Z (SEQ ID NO: 824), X-VLPQD-C(Sx)-EY*YKVKEPG-Z (SEQ ID NO: 825), X-YMEDSTYY*K-C(Sx)-SKGKL-Z (SEQ ID NO: 826), X-YRLSVEIY*D-C(Sx)-REYSR-Z (SEQ ID NO: 827), [Nle]EAIAKY*D-C(Sx)-KATAD-Z (SEQ ID NO: 828), X-AEPLPPSY*V-C(Sx)-AS-Z (SEQ ID NO: 829), X-AEPLP-C(Sx)-SY*VASS-Z (SEQ ID NO: 830), X-AIETDKEY*Y-C(Sx)-VKDDR-Z (SEQ ID NO: 831), X-AIYNSISY*K-C(Sx)-FLPK-Z (SEQ ID NO: 832), X-AVPEG-C(Sx)-EY*YRVREDG-Z (SEQ ID NO: 833), X-DGFIPKNY*I-C(Sx)-[Nle]KPHP-Z (SEQ ID NO: 834), X-DGPKGTGY*I-C(Sx)-TELIS-Z (SEQ ID NO: 835), X-DGPK-C(Sx)-TGY*IKTELIS-Z (SEQ ID NO: 836), X-DPGSA-C(Sx)-PY*LKTKFI-Z (SEQ ID NO: 733), X-DQTPL-C(Sx)-IY*NSISYKT-Z (SEQ ID NO: 837), X-DRLSQGAY*GG-C(Sx)-SDRP-Z (SEQ ID NO: 838), X-DRLSQ-C(Sx)-AY*GGLSDRP-Z (SEQ ID NO: 839), X-EESDQNWY*K-C(Sx)-ELNGK-Z (SEQ ID NO: 840), X-EESEDPDY*Y-C(Sx)-YNIQG-Z (SEQ ID NO: 841), X-ESQEELHY*A-C(Sx)-LNFPG-Z (SEQ ID NO: 842), X-EYLGTKRY*I-C(Sx)-RDLAT-Z (SEQ ID NO: 843), X-GSNQEEAY*V-C(Sx)-MSSFY-Z (SEQ ID NO: 844), X-GSVE[Nle]-C(Sx)-RY*DPLQDNT-Z (SEQ ID NO: 845), X-GVKKP-C(Sx)-RY*RPGTVAL-Z (SEQ ID NO: 846), X-HRIDGKIY*V-C(Sx)-KRVKY-Z (SEQ ID NO: 847), X-HTGFLTEY*V-C(Sx)-TRWYR-Z (SEQ ID NO: 782), X-HWKKYDIY*E-C(Sx)-QTKEE-Z (SEQ ID NO: 848), X-IDENSLLS*P-C(Sx)-AGEDD-Z (SEQ ID NO: 849), X-IETDKEYY*T-C(Sx)-KDDRD-Z (SEQ ID NO: 850), X-ILAIH-C(Sx)-SY*KPEFHSD-Z (SEQ ID NO: 851), X-ILLTIDRY*L-C(Sx)-IVHAV-Z (SEQ ID NO: 852), X-IPERDSRY*S-C(Sx)-PLHEE-Z (SEQ ID NO: 767), X-IRAKIQDY*H-C(Sx)-LTRKR-Z (SEQ ID NO: 853), X-IREFLDQY*D-C(Sx)-PL-Z (SEQ ID NO: 854), X-IREFL-C(Sx)-QY*DAPL-Z (SEQ ID NO: 855), X-KDISY-C(Sx)-RY*IPETLNK-Z (SEQ ID NO: 856), X-KFGSLDTY*L-C(Sx)-KNKN-Z (SEQ ID NO: 857), X-LAKAVDGY*V-C(Sx)-PQIKQ-Z (SEQ ID NO: 858), X-LAKA-C(Sx)-DGY*VKPQIKQ-Z (SEQ ID NO: 859), X-LILEDDDY*V-C(Sx)-VFFHQ-Z (SEQ ID NO: 860), X-LI[Nle]EY-C(Sx)-PY*GSLRDYL-Z (SEQ ID NO: 861), X-LLPLDKDY*Y-C(Sx)-VREPG-Z (SEQ ID NO: 862), X-LPLDKDYY*V-C(Sx)-REPGQ-Z (SEQ ID NO: 863), X-LPQDKEYY*K-C(Sx)-KEPGE-Z (SEQ ID NO: 807), X-[Nle]APPSEET*P-C(Sx)-IPQRS-Z (SEQ ID NO: 864), X-NHIGHTGY*L-C(Sx)-TVTVS-Z (SEQ ID NO: 865), X-NHIG-C(Sx)-TGY*LNTVTVS-Z (SEQ ID NO: 866), X-NLKLI-C(Sx)-EY*LPYGSLR-Z (SEQ ID NO: 867), X-NLQERRKY*L-C(Sx)-HRLI-Z (SEQ ID NO: 868), X-NLQER-C(Sx)-KY*LKHRLI-Z (SEQ ID NO: 869), X-NSKPKKSY*I-C(Sx)-TQG-Z (SEQ ID NO: 870), X-NSLFTPDY*E-C(Sx)-LTEND-Z (SEQ ID NO: 871), X-NSLISSDY*E-C(Sx)-LSDPT-Z (SEQ ID NO: 872), X-PEHTKSVY*T-C(Sx)-SVIEP-Z (SEQ ID NO: 873), X-PKGTQADY*A-C(Sx)-VKFQ-Z (SEQ ID NO: 874), X-PKGTQ-C(Sx)-DY*AEVKFQ-Z (SEQ ID NO: 875), X-PNEPVSDY*I-C(Sx)-ANI-Z (SEQ ID NO: 876), X-PPHLKYLY*L-C(Sx)-VSDSG-Z (SEQ ID NO: 877), X-PSSSIDEY*F-C(Sx)-EQPLK-Z (SEQ ID NO: 878), X-PTPPHLKY*L-C(Sx)-LVVSD-Z (SEQ ID NO: 879), X-PYGSLRDY*L-C(Sx)-KHKER-Z (SEQ ID NO: 880), X-QHDNIVKY*K-C(Sx)-VAYSA-Z (SEQ ID NO: 881), X-QISKG[Nle]EY*L-C(Sx)-SRRSV-Z (SEQ ID NO: 882), X-QISKG-C(Sx)-EY*LGTKRYI-Z (SEQ ID NO: 883), X-RHSTILDY*I-C(Sx)-VVPTA-Z (SEQ ID NO: 884), X-SFLSRSLY*F-C(Sx)-PLLHY-Z (SEQ ID NO: 885), X-SGGER-C(Sx)-DY*VAPSGSY-Z (SEQ ID NO: 886), X-SLRLV-C(Sx)-EY*LPSGSLR-Z (SEQ ID NO: 887), X-SPLTIGVY*V-C(Sx)-[Nle]PPTE-Z (SEQ ID NO: 888), X-TAKAVDGY*V-C(Sx)-PQIKQ-Z (SEQ ID NO: 889), X-TEQDL-C(Sx)-LY*SDFPNII-Z (SEQ ID NO: 890), X-TGMFPRNY*V-C(Sx)-PVNRN-Z (SEQ ID NO: 891), X-TPENN-C(Sx)-EY*AKVSGV[Nle]-Z (SEQ ID NO: 892), X-TSDEKVDY*V-C(Sx)-VDKEK-Z (SEQ ID NO: 893), X-VAKFIDDY*V-C(Sx)-VQIKQ-Z (SEQ ID NO: 894), X-VFLRSINY*V-C(Sx)-FPSLK-Z (SEQ ID NO: 895), X-VHPLSAPY*P-C(Sx)-LDTPE-Z (SEQ ID NO: 896), X-VHPLS-C(Sx)-PY*PPLDTPE-Z (SEQ ID NO: 897), X-VKYKGVAY*S-C(Sx)-GRRNL-Z (SEQ ID NO: 898), X-VLNQEEAY*V-C(Sx)-[Nle]ASFY-Z (SEQ ID NO: 899), X-VLPQD-C(Sx)-EY*YKVKEPG-Z (SEQ ID NO: 825), X-VLYRIRFY*F-C(Sx)-RWYSS-Z (SEQ ID NO: 900), X-VNGHLDSY*E-C(Sx)-VTQLP-Z (SEQ ID NO: 901), X-VPEGHEYY*R-C(Sx)-REDGD-Z (SEQ ID NO: 902), X-VRREVGDY*GQ-C(Sx)-HETE-Z (SEQ ID NO: 903), X-G[Nle]E-C(Sx)-IY*FEF[Nle]GGKKK-Z (SEQ ID NO: 904), X-G[Nle]EDIY*F-C(Sx)-F[Nle]GGKKK-Z (SEQ ID NO: 905), X-EAIY*A-C(Sx)-PFAKKK-Z (SEQ ID NO: 906), X-E-C(Sx)-IY*AAPFAKKK-Z (SEQ ID NO: 907); wherein X is H or acetyl; and Z is OH or NH2.

In some embodiments, the metal-binding compound of the present invention is selected from the group consisting of: X-RRAEEEEY*I-C(Sx)-LVA-Z (SEQ ID NO: 908), X-RAEEEEY*I-C(Sx)-LVA-Z (SEQ ID NO: 909), X-KKKAEEEEY*I-C(Sx)-L-Z (SEQ ID NO: 910), X-KKKAEEEEY*I-C(Sx)-Z (SEQ ID NO: 911), X-VYS-C(Sx)-DY*YRLFNPSKKK-Z (SEQ ID NO: 912), X-VYS-C(Sx)-DY*[pY]RLFNPSKKK-Z (SEQ ID NO: 913), X-GAGEE-C(Sx)-DY*[pY][pY]IWAGKKKK-Z (SEQ ID NO: 914), X-KKKK*E-C(Sx)-IYFFFG-Z (SEQ ID NO: 915), X-KKKRAHEEIY*F-C(Sx)-FWG-Z (SEQ ID NO: 916), X-KKKEEEIY*F-C(Sx)-FWG-Z (SEQ ID NO: 917), X-KKKKEEEIY*F-C(Sx)-FWG-Z (SEQ ID NO: 918), X-FTDRL-C(Sx)-QY*ISTRGLG-Z (SEQ ID NO: 919), X-FTDRLQQY*I-C(Sx)-TRGLG-Z (SEQ ID NO: 920), X-GC(Sx)-NY*RGYSLGNWV-Z (SEQ ID NO: 921), X-EEEIY*F-C(Sx)-FWG-Z (SEQ ID NO: 922), X-KKEEEIY*F-C(Sx)-FWG-Z (SEQ ID NO: 923), X-RAHEEIY*F-C(Sx)-FWG-Z (SEQ ID NO: 924); wherein X— is H or acetyl; and Z is OH or NH2.

In some embodiments, the metal-binding compound of the present invention is selected from the group consisting of: X-IENEEQEY*V-C(Sx)-TVKSS-Z (SEQ ID NO: 925), X-AVLADVSY*L-C(Sx)-AMEKS-Z (SEQ ID NO: 926), X-ARPLVEFY*E-C(Sx)-IKKYE-Z (SEQ ID NO: 927), X-FYEEIKKY*E-C(Sx)-LETEE-Z (SEQ ID NO: 928), X-DGDGQVNY*E-C(Sx)-FVQMM-Z (SEQ ID NO: 929), X-FDKDGNGY*I-C(Sx)-AAELR-Z (SEQ ID NO: 930), X-TQEQYELY*S-C(Sx)-MGSTF-Z (SEQ ID NO: 931), X-MAEEKKAY*K-C(Sx)-AKERE-Z (SEQ ID NO: 932), X-TDGEDADY*T-C(Sx)-FTNQQ-Z (SEQ ID NO: 933), X-SSHKGFHY*K-C(Sx)-G-Z (SEQ ID NO: 934), X-SSHKG-C(Sx)-HY*KH-Z (SEQ ID NO: 935), X-GGPEPGPY*A-C(Sx)-PSVNT-Z (SEQ ID NO: 936), X-RNEGVATY*A-C(Sx)-AVLFR-Z (SEQ ID NO: 937), X-DVVDADEY*L-C(Sx)-PQQGF-Z (SEQ ID NO: 938), X-DADEY*L-C(Sx)-PQQG-Z (SEQ ID NO: 939), X-EDSFLQRY*S-C(Sx)-DPTGA-Z (SEQ ID NO: 940), X-PVPEY*I-C(Sx)-QSV-Z (SEQ ID NO: 941), X-PVPEY*I-C(Sx)-QSV-Z (SEQ ID NO: 941), X-TFLPVPEY*I-C(Sx)-QSVPK-Z (SEQ ID NO: 942), X-TFLPV-C(Sx)-EY*INQSVPK-Z (SEQ ID NO: 943), X-ISLDNPDY*Q-C(Sx)-DFFPK-Z (SEQ ID NO: 944), X-AENAEY*L-C(Sx)-VA-Z (SEQ ID NO: 945), X-AENAEY*L-C(Sx)-VA-Z (SEQ ID NO: 945), X-STAENAEY*L-C(Sx)-VAPQS-Z (SEQ ID NO: 946), X-STAEN-C(Sx)-EY*LRVAPQS-Z (SEQ ID NO: 947), X-RLDGE-C(Sx)-IY*IRHSNLM-Z (SEQ ID NO: 948), X-NFANFSAY*P-C(Sx)-EEDMI-Z (SEQ ID NO: 949), X-KEVHKSGY*L-C(Sx)-SERLI-Z (SEQ ID NO: 950), X-LMLRLQDY*E-C(Sx)-KTKKA-Z (SEQ ID NO: 951), X-TEADGELY*V-C(Sx)-NTPSG-Z (SEQ ID NO: 952), X-MRHVSISY*D-C(Sx)-PPTPG-Z (SEQ ID NO: 953), X-SEELDENY*V-C(Sx)-MNPNS-Z (SEQ ID NO: 954), X-EPIQEANY*V-C(Sx)-MTPGT-Z (SEQ ID NO: 955), X-SHDSEENY*V-C(Sx)-MNPNL-Z (SEQ ID NO: 956), X-KGDKQVEY*L-C(Sx)-LDLDS-Z (SEQ ID NO: 957), X-VADERVDY*V-C(Sx)-VDQQK-Z (SEQ ID NO: 958), X-RNEEENIY*S-C(Sx)-PHDST-Z (SEQ ID NO: 959), X-SSTDR-C(Sx)-PY*EKVSAGN-Z (SEQ ID NO: 960), X-QLTFALRY*L-C(Sx)-FFTKA-Z (SEQ ID NO: 961), X-SSEPVGIY*Q-C(Sx)-FEKKT-Z (SEQ ID NO: 962), X-MQDNS-C(Sx)-TY*GKIWEGS-Z (SEQ ID NO: 963), X-TDVE-C(Sx)-TY*ADFIASG-Z (SEQ ID NO: 964), X-EGSFESRY*Q-C(Sx)-PFEDF-Z (SEQ ID NO: 965), X-IGTAE-C(Sx)-DY*GALYEGR-Z (SEQ ID NO: 966), X-EGRNPGFY*V-C(Sx)-ANPMP-Z (SEQ ID NO: 967), X-VTRRT-C(Sx)-DY*FL-Z (SEQ ID NO: 968), X-LHQEDNDY*I-C(Sx)-ASLIK-Z (SEQ ID NO: 969), X-LHQEDNDY*IN-C(Sx)-SLIK-Z (SEQ ID NO: 970), X-TVDGKEIY*N-C(Sx)-IRRKT-Z (SEQ ID NO: 971), X-TLMEKDSY*P-C(Sx)-FLKSP-Z (SEQ ID NO: 972), X-EEPPD-C(Sx)-QY*YNDFPGK-Z (SEQ ID NO: 973), X-EEPPD-C(Sx)-QY*[pY]NDFPGK-Z (SEQ ID NO: 974), X-EPPDHQYY*N-C(Sx)-FPGKE-Z (SEQ ID NO: 975), X-ELFDDPSY*V-C(Sx)-VQNLD-Z (SEQ ID NO: 795), X-GDGSD-C(Sx)-PY*YNSIPSK-Z (SEQ ID NO: 976), X-GDGSD-C(Sx)-PY*[pY]NSIPSK-Z (SEQ ID NO: 977), X-DGSDHPYY*N-C(Sx)-IPSKM-Z (SEQ ID NO: 978), X-FAGKE-C(Sx)-TY*YQGRHLG-Z (SEQ ID NO: 979), X-FAGKE-C(Sx)-TY*[pY]QGRHLG-Z (SEQ ID NO: 980), X-AGKEQTYY*Q-C(Sx)-RHLGD-Z (SEQ ID NO: 981), X-RQGSSDIY*S-C(Sx)-PEGKL-Z (SEQ ID NO: 982), X-PTGEAPTY*V-C(Sx)-TQQIP-Z (SEQ ID NO: 983), X-TENDDDVY*R-C(Sx)-LEELA-Z (SEQ ID NO: 984), X-HDLGEDIY*D-C(Sx)-VP-Z (SEQ ID NO: 985), X-HDLGEDIY*D-C(Sx)-VP-Z (SEQ ID NO: 985), X-EDGGDDIY*E-C(Sx)-IIKVE-Z (SEQ ID NO: 986), X-EDGGDDIY*E-C(Sx)-IIKVE-Z (SEQ ID NO: 986), X-RNEGVATY*A-C(Sx)-AVLFR-Z (SEQ ID NO: 937), X-SPQPEY*V-C(Sx)-QPDVR-Z (SEQ ID NO: 987), X-SPAFD-C(Sx)-LY*YWDQDPP-Z (SEQ ID NO: 988), X-SPAFD-C(Sx)-LY*[pY]WDQDPP-Z (SEQ ID NO: 989), X-PAFDNLYY*W-C(Sx)-QDPPE-Z (SEQ ID NO: 990), X-PTAENPEY*L-C(Sx)-LDVPV-Z (SEQ ID NO: 991), X-QALDNPEY*H-C(Sx)-ASNGP-Z (SEQ ID NO: 992); wherein X— is H or acetyl; and Z is OH or NH2.

In some embodiments, the metal-binding compound of the present invention is selected from the group consisting of: X-RRRAEEEEY*I-C(Sx)-LVA-Z (SEQ ID NO: 993), X-KKKAEEEEY*I-C(Sx)-LVA-Z (SEQ ID NO: 994), X-KKAEEEEY*I-C(Sx)-LVA-Z (SEQ ID NO: 995), X-KAEEEEY*I-C(Sx)-LVA-Z (SEQ ID NO: 996), X-EEPIY*V-C(Sx)-VP-Z (SEQ ID NO: 549), X-EEPIY*V-C(Sx)-IK-Z (SEQ ID NO: 536), X-EEPIY*V-C(Sx)-[Nle]P-Z (SEQ ID NO: 530), X-EEPIY*I-C(Sx)-VK-Z (SEQ ID NO: 499), X-EEPIY*V-C(Sx)-VK-Z (SEQ ID NO: 547), X-EEPIY*I-C(Sx)-WP-Z (SEQ ID NO: 503), X-EEPIY*V-C(Sx)-WK-Z (SEQ ID NO: 550), X-EEPDY*I-C(Sx)-VP-Z (SEQ ID NO: 382), X-EEPEY*I-C(Sx)-VP-Z (SEQ ID NO: 418), X-EEPDY*V-C(Sx)-MK-Z (SEQ ID NO: 390), X-DEQIY*W-C(Sx)-IA-Z (SEQ ID NO: 31), X-KKKDEQIY*W-C(Sx)-IA-Z (SEQ ID NO: 997), X-SSKRAKAKTTKKRPQRATS*N-C(Sx)-FS-Z (SEQ ID NO: 998), X-KAKTTKKRPQRATS*N-C(Sx)-FS-Z (SEQ ID NO: 999), X-KTTKKRPQRATS*N-C(Sx)-FS-Z (SEQ ID NO: 1000), X-KKRPQRATS*N-C(Sx)-FAM-Z (SEQ ID NO: 145), X-QETFS*D-C(Sx)-WKLLP-Z (SEQ ID NO: 1001), X-QE-C(Sx)-FS*DLWKLLP-Z (SEQ ID NO: 1002), X-VSGQL-C(Sx)-DS*MANSFVGTRSYKKK-Z (SEQ ID NO: 1003), X-KKKVSGQLIDS*M-C(Sx)-NSFVGTRSY-Z (SEQ ID NO: 1004), X-KKKVSGQLIDSM-C(Sx)-NS*FVGTRSY-Z (SEQ ID NO: 1005), X-KKKVSGQLIDSMANS*F-C(Sx)-GTRSY-Z (SEQ ID NO: 1006), X-KKCVSGQLIDS*M-C(Sx)-NSFVGTRSYCKK-Z (SEQ ID NO: 1007), X-KKCVSGQLIDSM-C(Sx)-NS*FVGTRSYCKK-Z (SEQ ID NO: 1008), X-GYAKDV-C(Sx)-QGS*LCTSFVGTLQYLAKKK-Z (SEQ ID NO: 1009), X-GYAKDVDQGS*L-C(Sx)-TSFVGTLQYLAKKK-Z (SEQ ID NO: 1010), X-GYAKDVDQGSL-C(Sx)-TS*FVGTLQYLAKKK-Z (SEQ ID NO: 1011), X-GYAKDVDQGSLCTS*F-C(Sx)-GTLQYLA-Z (SEQ ID NO: 1012), X-DLGYAKDV-C(Sx)-QGS*LCTSFVGTLQYLAPEKKK-Z (SEQ ID NO: 1013), X-DLGYAKDVDQGS*L-C(Sx)-TSFVGTLQYLAPE-Z (SEQ ID NO: 1014), X-DLGYAKDVDQGSL-C(Sx)-TS*FVGTLQYLAPE-Z (SEQ ID NO: 1015), X-DLGYAKDVDQGSLCTS*F-C(Sx)-GTLQYLAPE-Z (SEQ ID NO: 1016), X-KKCDLGYAKDVDQGS*L-C(Sx)-TSFVGTLQYLAPECKK-Z (SEQ ID NO: 1017), X-KKCDLGYAKDVDQGSL-C(Sx)-TS*FVGTLQYLAPECKK-Z (SEQ ID NO: 1018), X-C(Sx)LS*FRRR-Z (SEQ ID NO: 1019), X-GKKALRRQES*V-C(Sx)-ALGW-Z (SEQ ID NO: 1020), X-GNLLPMS*P-C(Sx)-EFD-Z (SEQ ID NO: 1021); wherein X— is H or acetyl; and Z is OH or NH2.

In some embodiments, the metal-binding compound of the present invention is selected from the group consisting of: X-IPTS*P-C(Sx)-TITYFFF-Z (SEQ ID NO: 209), X-I-C(Sx)-TS*PITITYFFF-Z (SEQ ID NO: 210), X-IPTS*P-C(Sx)-TITYFFFKKK-Z (SEQ ID NO: 211), X-VADQTPT*P-C(Sx)-RFLKN-Z (SEQ ID NO: 1022), X-VA-C(Sx)-QT*P[pT]PTRFLKN-Z (SEQ ID NO: 1023), X-IDQGDLMT*P-C(Sx)-FTPYY-Z (SEQ ID NO: 1024), X-IDQGD-C(Sx)-MT*PQFTPYY-Z (SEQ ID NO: 1025), X-TPPEELPS*P-C(Sx)-ASSLG-Z (SEQ ID NO: 1026), X-TPPEE-C(Sx)-PS*PSASSLG-Z (SEQ ID NO: 1027), X-NTIDLPMS*P-C(Sx)-TLDSL-Z (SEQ ID NO: 1028), X-NTIDL-C(Sx)-MS*PRTLDSL-Z (SEQ ID NO: 1029), X-KKKESTSAPLS*P-C(Sx)-VTITSP-Z (SEQ ID NO: 1030), X-GFLSRPLS*P-C(Sx)-FTTSP-Z (SEQ ID NO: 106), X-PSLLRPLS*P-C(Sx)-FFGAY-Z (SEQ ID NO: 107), X-KKKSTPSSPSS*P-C(Sx)-SSVAY-Z (SEQ ID NO: 1031), X-KKKSHGSAPLS*P-C(Sx)-APLTS-Z (SEQ ID NO: 1032), X-KKKSPFSVLSS*P-C(Sx)-SGHSN-Z (SEQ ID NO: 1033), X-TEERLPSS*P-C(Sx)-YEDAA-Z (SEQ ID NO: 1034), X-TEERL-C(Sx)-SS*PVYEDAA-Z (SEQ ID NO: 1035), X-SNGRRKRS*P-C(Sx)-QSFRF-Z (SEQ ID NO: 1036), X-SNGRR-C(Sx)-RS*PTQSFRF-Z (SEQ ID NO: 1037), X-EENVSDGS*P-C(Sx)-AGSVE-Z (SEQ ID NO: 1038), X-EENV-C(Sx)-DGS*PNAGSVE-Z (SEQ ID NO: 1039), X-APEKGLPT*P-C(Sx)-VLQRN-Z (SEQ ID NO: 1040), X-APEKG-C(Sx)-PT*PQVLQRN-Z (SEQ ID NO: 1041), X-LIDATGDT*P-C(Sx)-AEDDE-Z (SEQ ID NO: 1042), X-LIDAT-C(Sx)-DT*PGAEDDE-Z (SEQ ID NO: 1043), X-VEVDPMLT*P-C(Sx)-ERHLN-Z (SEQ ID NO: 1044), X-VEVDP-C(Sx)-LT*PEERHLN-Z (SEQ ID NO: 1045), X-EEYETPES*P-C(Sx)-PPARS-Z (SEQ ID NO: 1046), X-EEYET-C(Sx)-ES*PVPPARS-Z (SEQ ID NO: 1047), X-PAADAIMS*P-C(Sx)-EELDG-Z (SEQ ID NO: 1048), X-PAADA-C(Sx)-MS*PEEELDG-Z (SEQ ID NO: 1049), X-VLSRQITS*P-C(Sx)-SGING-Z (SEQ ID NO: 1050), X-VLSRQ-C(Sx)-TS*PVSGING-Z (SEQ ID NO: 1051), X-RSKSAPTS*P-C(Sx)-DQEIK-Z (SEQ ID NO: 1052), X-TPPQEHIS*P-C(Sx)-ITNEV-Z (SEQ ID NO: 1053), X-TPPQE-C(Sx)-IS*PQITNEV-Z (SEQ ID NO: 1054), X-LSTPVVLS*P-C(Sx)-PQKP-Z (SEQ ID NO: 1055), X-LSTPV-C(Sx)-LS*PGPQKP-Z (SEQ ID NO: 1056), X-QLSKWPGS*P-C(Sx)-SRSSD-Z (SEQ ID NO: 1057), X-KNIVTPRT*P-C(Sx)-PSQGK-Z (SEQ ID NO: 1058), X-KNIVT-C(Sx)-RT*PPPSQGK-Z (SEQ ID NO: 1059), X-SGVEVRVT*P-C(Sx)-RTEII-Z (SEQ ID NO: 1060), X-SGVEV-C(Sx)-VT*PTRTEII-Z (SEQ ID NO: 1061), X-SRRSGLSS*P-C(Sx)-YVAVT-Z (SEQ ID NO: 1062), X-SRRSG-C(Sx)-SS*PSYVAVT-Z (SEQ ID NO: 1063), X-TFDSLPSS*P-C(Sx)-SATPH-Z (SEQ ID NO: 1064), X-TFDSL-C(Sx)-SS*PSSATPH-Z (SEQ ID NO: 1065), X-PVVSGDTS*P-C(Sx)-HLSNV-Z (SEQ ID NO: 1066), X-PVVSG-C(Sx)-TS*PRHLSNV-Z (SEQ ID NO: 1067); wherein X— is H or acetyl; and Z is OH or NH2.

In some embodiments, the metal-binding compound of the present invention is selected from the group consisting of: X-PTIPGVTS*P-C(Sx)-SDEPP-Z (SEQ ID NO: 1068), X-SRSHSAKT*P-C(Sx)-FSVQS-Z (SEQ ID NO: 1069), X-GHVEEPAS*P-C(Sx)-AAYQK-Z (SEQ ID NO: 1070), X-QGPAPVGT*P-C(Sx)-FNRQH-Z (SEQ ID NO: 1071), X-LLSNASAT*P-C(Sx)-GRRGR-Z (SEQ ID NO: 1072), X-QARAKTQT*P-C(Sx)-VSPAP-Z (SEQ ID NO: 1073), X-QARAK-C(Sx)-QT*PPVSPAP-Z (SEQ ID NO: 1074), X-VSYEDPPT*A-C(Sx)-AAVEW-Z (SEQ ID NO: 1075), X-QMAGTPIT*P-C(Sx)-KDGFT-Z (SEQ ID NO: 1076), X-QMAGTPIT*P-C(Sx)-KDGFT-Z (SEQ ID NO: 1076), X-DADRSILS*P-C(Sx)-GSCGP-Z (SEQ ID NO: 1077), X-LVEKLPDS*P-C(Sx)-LAKKA-Z (SEQ ID NO: 1078), X-FGESPPLS*P-C(Sx)-DMDTQ-Z (SEQ ID NO: 1079), X-EVPDVTAT*P-C(Sx)-RLLFF-Z (SEQ ID NO: 1080), X-SAVNGTSS*A-C(Sx)-TNLEA-Z (SEQ ID NO: 1081), X-LGGLRISS*D-C(Sx)-SSDIE-Z (SEQ ID NO: 1082), X-LAGISRS*V-C(Sx)-VTVAY-Z (SEQ ID NO: 1083), X-NPQDSVGS*P-C(Sx)-SRVGE-Z (SEQ ID NO: 1084), X-KDEILPT*P-C(Sx)-SEQKG-Z (SEQ ID NO: 1085), X-KDEIL-C(Sx)-T*PISEQKG-Z (SEQ ID NO: 1086), X-HQLFRGFS*F-C(Sx)-AITSD-Z (SEQ ID NO: 1087), X-RGGPEPLS*P-C(Sx)-PVSPL-Z (SEQ ID NO: 1088), X-SGDYMPMS*P-C(Sx)-SVSAP-Z (SEQ ID NO: 1089), X-YMPMSPKS*V-C(Sx)-APQQI-Z (SEQ ID NO: 1090), X-QAATGFGS*P-C(Sx)-QYSAD-Z (SEQ ID NO: 1091), X-SFITPPIT*P-C(Sx)-LRRHT-Z (SEQ ID NO: 1092), X-TKLKPPRT*P-C(Sx)-PPSRK-Z (SEQ ID NO: 1093), X-ESPEKVS*P-C(Sx)-KICDF-Z (SEQ ID NO: 1094), X-GLSGRKSS*T-C(Sx)-SPTSP-Z (SEQ ID NO: 1095), X-PSVNPSIS*P-C(Sx)-HGVAR-Z (SEQ ID NO: 1096), X-YKRRYVET*P-C(Sx)-VHISS-Z (SEQ ID NO: 1097), X-NVRVSNGS*P-C(Sx)-LERMD-Z (SEQ ID NO: 1098), X-DDIEQWFT*E-C(Sx)-PGPDE-Z (SEQ ID NO: 1099), X-EPPPRPKT*P-C(Sx)-IFRAL-Z (SEQ ID NO: 1100), X-EPPPRPKT*P-C(Sx)-IFRAL-Z (SEQ ID NO: 1100), X-ARVSPPES*P-C(Sx)-ARAAA-Z (SEQ ID NO: 1101), X-ARVSPPES*P-C(Sx)-ARAAA-Z (SEQ ID NO: 1101), X-KKKLAPNPSFS*P-C(Sx)-PGFTP-Z (SEQ ID NO: 1102); wherein X— is H or acetyl; and Z is OH or NH2.

In some embodiments, the metal-binding compound of the present invention is selected from the group consisting of: X-EIRSRHSS*Y-C(Sx)-AGTED-Z (SEQ ID NO: 1103), X-SRDPVART*S-C(Sx)-LQTPA-Z (SEQ ID NO: 1104), X-PSWHLADS*P-C(Sx)-VNGAT-Z (SEQ ID NO: 1105), X-PTSPRRYS*P-C(Sx)-AKDLL-Z (SEQ ID NO: 1106), X-IHFWSSLS*P-C(Sx)-APLSP-Z (SEQ ID NO: 1107), X-LSPVAPLS*P-C(Sx)-RLQGP-Z (SEQ ID NO: 1108), X-PQPLRS*P-C(Sx)-LDNPT-Z (SEQ ID NO: 1109), X-GSASASGS*P-C(Sx)-DPGFM-Z (SEQ ID NO: 1110), X-RTDSLAAT*P-C(Sx)-AAK-Z (SEQ ID NO: 1111), X-RRVSGNNS*P-C(Sx)-LSNGG-Z (SEQ ID NO: 1112), X-RPASVNGS*P-C(Sx)-ATSES-Z (SEQ ID NO: 1113), X-PSRPPPPT*P-C(Sx)-RPASV-Z (SEQ ID NO: 1114), X-DRVSRSSS*P-C(Sx)-KTGEQ-Z (SEQ ID NO: 1115), X-PLKTGEQT*P-C(Sx)-HEHI-Z (SEQ ID NO: 1116), X-ATEEIYLT*P-C(Sx)-QRPPD-Z (SEQ ID NO: 1117), X-SNGH IT*P-C(Sx)-PTQFL-Z (SEQ ID NO: 1118), X-GHITITPT*P-C(Sx)-QFL-Z (SEQ ID NO: 1119), X-KKKSNGLVTIT*P-C(Sx)-SSQFL-Z (SEQ ID NO: 1120), X-PSAPALST*P-C(Sx)-IRDSA-Z (SEQ ID NO: 1121), X-SIMQQ-C(Sx)-DS*PHVVKYY-Z (SEQ ID NO: 1122), X-EYSLPALT*P-C(Sx)-LDEVK-Z (SEQ ID NO: 1123), X-RGSMAAPS*L-C(Sx)-PSLGP-Z (SEQ ID NO: 1124), X-KRKLP-C(Sx)-DT*PGQGLT-Z (SEQ ID NO: 1125), X-PRTSPIMS*P-C(Sx)-TSLAE-Z (SEQ ID NO: 1126), X-GKKATQAS*Q-C(Sx)-Y-Z (SEQ ID NO: 1127), X-NSKYNAES*T-C(Sx)-RESQD-Z (SEQ ID NO: 1128), X-SKYNAES*T-C(Sx)-RESQDT-Z (SEQ ID NO: 1129), X-SGYSSPGS*P-C(Sx)-TPGSR-Z (SEQ ID NO: 1130), X-GQSSAAAT*P-C(Sx)-TTGTK-Z (SEQ ID NO: 1131), X-ITGTKSNT*P-C(Sx)-SSVPS-Z (SEQ ID NO: 1132), X-TTGTK-C(Sx)-NT*PTSSVPS-Z (SEQ ID NO: 1133), X-SVPSAAVT*P-C(Sx)-NESLQ-Z (SEQ ID NO: 1134); wherein X— is H or acetyl; and Z is OH or NH2.

In some embodiments, the metal-binding compound of the present invention is selected from the group consisting of: X-SNTGQAIS*P-C(Sx)-MKRRI-Z (SEQ ID NO: 1135), X-SNTGQ-C(Sx)-IS*PGMKRRI-Z (SEQ ID NO: 1136), X-QSMVVPQT*P-C(Sx)-HTARV-Z (SEQ ID NO: 1137), X-QSMVV-C(Sx)-QT*PLHTARV-Z (SEQ ID NO: 1138), X-TTLHRNVS*P-C(Sx)-APQRP-Z (SEQ ID NO: 1139), X-TTLHR-C(Sx)-VS*PGAPQRP-Z (SEQ ID NO: 1140), X-SIKSEPIS*P-C(Sx)-RDRMT-Z (SEQ ID NO: 1141), X-SIKSE-C(Sx)-IS*PPRDRMT-Z (SEQ ID NO: 1142), X-DPRGDFHS*P-C(Sx)-VLGRP-Z (SEQ ID NO: 1143), X-DPRGD-C(Sx)-HS*PIVLGRP-Z (SEQ ID NO: 1144), X-PNTEDRES*P-C(Sx)-VKRMR-Z (SEQ ID NO: 1145), X-PNTED-C(Sx)-ES*PSVKRMR-Z (SEQ ID NO: 1146), X-KEHRG-C(Sx)-DS*PDPDTSY-Z (SEQ ID NO: 1147), X-PDTSYVLT*P-C(Sx)-TEEKY-Z (SEQ ID NO: 1148), X-PDTSY-C(Sx)-LT*PHTEEKY-Z (SEQ ID NO: 1149), X-SAKSR-C(Sx)-QT*APVPMPD-Z (SEQ ID NO: 1150), X-SAKSR-C(Sx)-QT*APVPMPD-Z (SEQ ID NO: 1150), X-STSVT-C(Sx)-HS*PSSPVGS-Z (SEQ ID NO: 1151), X-KMEVEELS*P-C(Sx)-ALGRF-Z (SEQ ID NO: 1152), X-S*T-C(Sx)-SEPLSPTSSLGE-Z (SEQ ID NO: 1153), X-GVRRRRLS*N-C(Sx)-SLTGV-Z (SEQ ID NO: 1154), X-GVRRR-C(Sx)-LS*NVSLTGV-Z (SEQ ID NO: 1155), X-RRRLSNVS*L-C(Sx)-GVSTI-Z (SEQ ID NO: 1156), X-RRRLS-C(Sx)-VS*LTGVSTI-Z (SEQ ID NO: 1157), X-GREKRSNS*Q-C(Sx)-YIGRP-Z (SEQ ID NO: 1158), X-GREKR-C(Sx)-NS*QSYIGRP-Z (SEQ ID NO: 1159), X-EDANRTIS*P-C(Sx)-TSNNI-Z (SEQ ID NO: 1160), X-EDANRTISP-C(Sx)-TS*NNI-Z (SEQ ID NO: 1161), X-RTISPSTS*N-C(Sx)-IPLMR-Z (SEQ ID NO: 1162), X-RTISP-C(Sx)-TS*NNIPLMR-Z (SEQ ID NO: 1163), X-GEKSFRRS*V-C(Sx)-GTPAY-Z (SEQ ID NO: 1164), X-GEKSF-C(Sx)-RS*VVGTPAY-Z (SEQ ID NO: 1165), X-EPGPVPSS*P-C(Sx)-QEPPT-Z (SEQ ID NO: 1166), X-PLQRQPSS*P-C(Sx)-PTPRN-Z (SEQ ID NO: 1167); wherein X— is H or acetyl; and Z is OH or NH2.

In some embodiments, the metal-binding compound of the present invention is selected from the group consisting of: X-YSHKGHLS*E-C(Sx)-LVTKW-Z (SEQ ID NO: 1168), X-PPYNRAVS*L-C(Sx)-SPVSV-Z (SEQ ID NO: 1169), X-PDVEMPS*P-C(Sx)-APSGD-Z (SEQ ID NO: 1170), X-SRLRD-C(Sx)-PS*APLEAPE-Z (SEQ ID NO: 1171), X-DPLPP-C(Sx)-FS*GTPKGSG-Z (SEQ ID NO: 1172), X-VKAEPAHT*A-C(Sx)-SVAAK-Z (SEQ ID NO: 1173), X-DSLSYYHS*P-C(Sx)-DSFSS-Z (SEQ ID NO: 1174), X-SALQGFNS*P-C(Sx)-MLSLG-Z (SEQ ID NO: 1175), X-LTDPRLLS*P-C(Sx)-QPALQ-Z (SEQ ID NO: 1176), X-VNTRA-C(Sx)-PS*QHSSPAV-Z (SEQ ID NO: 1177), X-QHSSPAVS*D-C(Sx)-LPSNS-Z (SEQ ID NO: 1178), X-AVSDSLPS*N-C(Sx)-LKKSS-Z (SEQ ID NO: 1179), X-SNSLKKSS*A-C(Sx)-LKKIL-Z (SEQ ID NO: 1180), X-LPGPPS*P-C(Sx)-SDAES-Z (SEQ ID NO: 1181), X-SPLSDPSS*P-C(Sx)-ASEDF-Z (SEQ ID NO: 1182), X-ANPSPPPS*P-C(Sx)-QQINL-Z (SEQ ID NO: 1183), X-AHEGV-C(Sx)-LS*VPEYRSR-Z (SEQ ID NO: 1184), X-EKGPEGVS*P-C(Sx)-QAHLH-Z (SEQ ID NO: 1185), X-GPEDQAVT*E-C(Sx)-VATRW-Z (SEQ ID NO: 1186), X-GGSSGT*S-C(Sx)-EKGPE-Z (SEQ ID NO: 1187), X-DPQLPSRT*P-C(Sx)-QGPRP-Z (SEQ ID NO: 1188), X-APEVL-C(Sx)-SS*HRYTLGV-Z (SEQ ID NO: 1189), X-RADPQ-C(Sx)-PS*RTPVQGP-Z (SEQ ID NO: 1190), X-SSKRAKAKTTKKRPQRATS*N-C(Sx)-FS-Z (SEQ ID NO: 998), X-KAKTTKKRPQRATS*N-C(Sx)-FS-Z (SEQ ID NO: 999), X-KTTKKRPQRATS*N-C(Sx)-FS-Z (SEQ ID NO: 1000), X-KKRPQRATS*N-C(Sx)-FAM-Z (SEQ ID NO: 145), X-QETFS*D-C(Sx)-WKLLP-Z (SEQ ID NO: 1001), X-QE-C(Sx)-FS*DLWKLLP-Z (SEQ ID NO: 1002), X-GYAKDVDQGSL-C(Sx)-TS*FVGTLQYLAKKK-Z (SEQ ID NO: 1011), X-GYAKDVDQGS*L-C(Sx)-TSFVGTLQYLAKKK-Z (SEQ ID NO: 1010), X-R-C(Sx)-FS*LRRKAK-Z (SEQ ID NO: 1191), X-A-C(Sx)-KTPKKAKKP-Z (SEQ ID NO: 1192), X-I-C(Sx)-TS*PITI-YFFFKKK-Z (SEQ ID NO: 1193), X-WD-C(Sx)-DS*DDDDDAAA-Z (SEQ ID NO: 1194), X-WD-C(Sx)-DS*DDDDDAAAKKK-Z (SEQ ID NO: 1195), X-VSGQL-C(Sx)-DS*MANSFVGTRSYKKK-Z (SEQ ID NO: 1003), X-KKKVSGQLIDSM-C(Sx)-NS*FVGTRSY-Z (SEQ ID NO: 1005), KKKVSGQLIDS*M-C(Sx)-NSFVGTRSY-Z (SEQ ID NO: 1004), X-KKKVSGQLIDSMANSF-C(Sx)-GT*RSY-Z (SEQ ID NO: 1196), X-KKKVSGQLIDSMANS*F-C(Sx)-GTRSY-Z (SEQ ID NO: 1006), X-QYHFVASSST*I-C(Sx)-RDRQRPYS-Z (SEQ ID NO: 1197), X-QYHFVAS-C(Sx)-ST*IERDRQRPYS-Z (SEQ ID NO: 1198), X-DANPLMANGT*L-C(Sx)-RRHQNGRF-Z (SEQ ID NO: 1199), X-DANPLMA-C(Sx)-GT*LTRRHQNGRF-Z (SEQ ID NO: 1200), X-DANPLM-C(Sx)-NGT*LTRRHQNGRF-Z (SEQ ID NO: 1201), X-DANPLMA-C(Sx)-GT*LARRHQNGRF-Z (SEQ ID NO: 1202), X-DANPLM-C(Sx)-NGT*LARRHQNGRF-Z (SEQ ID NO: 1203); wherein X— is H or acetyl; and Z is OH or NH2.

In some embodiments, the metal-binding compound of the present invention is selected from the group consisting of: X-[Nle]AKTTKKRPQRATS*N-C(Sx)-FS-Z (SEQ ID NO: 1204), X-[Nle]AKITKKRPQRATSN-C(Sx)-FS*-Z (SEQ ID NO: 1205), X-[Nle]ANPLMA-C(Sx)-GT*LTRRHQNGRF-Z (SEQ ID NO: 1206), X-[Nle]-C(Sx)-DS*LRRKAK-Z (SEQ ID NO: 1207), X-[Nle]-C(Sx)-LS*PGPF-Z (SEQ ID NO: 1208), X-[Nle]KRPQRAT*S-C(Sx)-VFAMF-Z (SEQ ID NO: 1209), X-[Nle]LLR-C(Sx)-SS*RRIRR-Z (SEQ ID NO: 1210), X-[Nle]LPWWR-C(Sx)-YT*WVVERDVNTKQR-Z (SEQ ID NO: 1211), X-[Nle]QREGFGRQS*M-C(Sx)-EKR-Z (SEQ ID NO: 1212), X-[Nle]RR-C(Sx)-AS*FRRR-Z (SEQ ID NO: 1213), X-AA-C(Sx)-IS*PLGALQHGFFR-Z (SEQ ID NO: 1214), X-AANPLMA-C(Sx)-GT*LTRRHQNGRF-Z (SEQ ID NO: 1215), X-A-C(Sx)-DS*LRRKAK-Z (SEQ ID NO: 1216), X-A-C(Sx)-IS*PLGALQHGFFR-Z (SEQ ID NO: 1217), X-A-C(Sx)-KT*PKKAKKP-Z (SEQ ID NO: 1218), X-A-C(Sx)-LS*PGPF-Z (SEQ ID NO: 1219), X-ADPDHDHT*G-C(Sx)-LTEYVATRWRR-Z (SEQ ID NO: 1220), X-ADPDHDHTG-C(Sx)-LT*EYVATRWRR-Z (SEQ ID NO: 1221), X-AE-C(Sx)-QS*LNAADVENLHLPL-Z (SEQ ID NO: 1222), X-AESQS*L-C(Sx)-AADVENLHLPL-Z (SEQ ID NO: 1223), X-AFRLER-C(Sx)-DS*IFYPRR-Z (SEQ ID NO: 1224), X-AGTS*F-C(Sx)-[Nle]TP[pY]VVTRKKK-Z (SEQ ID NO: 1225), X-AGTS*F-C(Sx)-[Nle]TPYVVTRKKK-Z (SEQ ID NO: 1226), X-AGTSF-C(Sx)-[Nle]T*P[pY]VVTRKKK-Z (SEQ ID NO: 1227), X-AGTSF-C(Sx)-[Nle]T*PYVVTRKKK-Z (SEQ ID NO: 1228), X-AHEGV-C(Sx)-LS*VPEYRSR-Z (SEQ ID NO: 1184), X-AHLQRQLS*I-C(Sx)-EN-Z (SEQ ID NO: 1229), X-AHLQRQLS*I-C(Sx)-EP-Z (SEQ ID NO: 1230), X-AHLQRQLS*I-C(Sx)-FA-Z (SEQ ID NO: 1231), X-AHLQRQLS*I-C(Sx)-FS-Z (SEQ ID NO: 1232), X-AHLQRQLS*I-C(Sx)-HH-Z (SEQ ID NO: 1233), X-AHLQRQLSI-C(Sx)-FS*-Z (SEQ ID NO: 1234), X-AKRPQRAT*S-C(Sx)-VFA-Z (SEQ ID NO: 1235), X-AKRPQRAT*S-C(Sx)-VF-Z (SEQ ID NO: 1236), X-AKRPQRATS*N-C(Sx)-FA-Z (SEQ ID NO: 1237), X-AKRPQRATS*N-C(Sx)-F-Z (SEQ ID NO: 1238), X-AKRRRLAS*L-C(Sx)-ASTSK-Z (SEQ ID NO: 130), X-AKRRRLASL-C(Sx)-AS*TSK-Z (SEQ ID NO: 1239), X-ALKLSRYS*F-C(Sx)-ITAK-Z (SEQ ID NO: 1240), X-ALKLSRYSF-C(Sx)-IT*AK-Z (SEQ ID NO: 1241), X-ALLR-C(Sx)-SS*RRIRR-Z (SEQ ID NO: 1242), X-ALPWWR-C(Sx)-YT*WVVERDVNTKQR-Z (SEQ ID NO: 1243), X-ALRRAS*L-C(Sx)-AA-Z (SEQ ID NO: 1244), X-ALRRFS*L-C(Sx)-GA-Z (SEQ ID NO: 1245), X-AP-C(Sx)-PS*QAMDDLMLSPDD-Z (SEQ ID NO: 1246), and X-APEKG-C(Sx)-PT*PQVLQRN-Z (SEQ ID NO: 1041); wherein X is H or acetyl; and Z is OH or NH2.

In some embodiments, the metal-binding compound of the present invention is selected from the group consisting of: X-APEKGLPT*P-C(Sx)-VLQRN-Z (SEQ ID NO: 1040), X-APEVL-C(Sx)-SS*HRYTLGV-Z (SEQ ID NO: 1189), X-APPSEET*P-C(Sx)-IPQRS-Z (SEQ ID NO: 1247), X-AQAEGFGRQS*[Nle]-C(Sx)-EKR[pT]K-Z (SEQ ID NO: 1248), X-AQAEGFGRQS*[Nle]-C(Sx)-EKRTK-Z (SEQ ID NO: 1249), X-AQAEGFGRQS*M-C(Sx)-EKR[pT]K-Z (SEQ ID NO: 1250), X-AQAEGFGRQS*M-C(Sx)-EKRTK-Z (SEQ ID NO: 1251), X-AQREGFGRQS*M-C(Sx)-EKR-Z (SEQ ID NO: 1252), X-AR-C(Sx)-LS*FAEG-Z (SEQ ID NO: 1253), X-AR-C(Sx)-LS*FAEPG-Z (SEQ ID NO: 1254), X-ARKRERAYS*[Nle][dP]-C(Sx)-G-Z (SEQ ID NO: 1255), X-ARKRERAYS*[Nle]-C(Sx)-G-Z (SEQ ID NO: 1256), X-ARKRERAYS*A-C(Sx)-G-Z (SEQ ID NO: 1257), X-ARKRERAYS*E[dP]-C(Sx)-G-Z (SEQ ID NO: 1258), X-ARKRERAYS*F[dP]-C(Sx)-G-Z (SEQ ID NO: 1259), X-ARKRERAYS*FA-C(Sx)-G-Z (SEQ ID NO: 1260), X-ARKRERAYS*F-C(Sx)-G-Z (SEQ ID NO: 1261), X-ARKRERAYS*FD-C(Sx)-G-Z (SEQ ID NO: 1262), X-ARKRERAYS*FE-C(Sx)-G-Z (SEQ ID NO: 1263), X-ARKRERAYS*FF-C(Sx)-G-Z (SEQ ID NO: 1264), X-ARKRERAYS*FG-C(Sx)-G-Z (SEQ ID NO: 1265), X-ARKRERAYS*FH-C(Sx)-G-Z (SEQ ID NO: 1266), X-ARKRERAYS*FI-C(Sx)-G-Z (SEQ ID NO: 1267), X-ARKRERAYS*FK-C(Sx)-G-Z (SEQ ID NO: 1268), X-ARKRERAYS*FL-C(Sx)-G-Z (SEQ ID NO: 1269), X-ARKRERAYS*FN-C(Sx)-G-Z (SEQ ID NO: 1270), X-ARKRERAYS*FP-C(Sx)-G-Z (SEQ ID NO: 1271), X-ARKRERAYS*FQ-C(Sx)-G-Z (SEQ ID NO: 1272), X-ARKRERAYS*FR-C(Sx)-G-Z (SEQ ID NO: 1273), X-ARKRERAYS*FV-C(Sx)-G-Z (SEQ ID NO: 1274), X-ARKRERAYS*FW-C(Sx)-G-Z (SEQ ID NO: 1275), X-ARKRERAYS*G[dP]-C(Sx)-G-Z (SEQ ID NO: 1276), X-ARKRERAYS*G-C(Sx)-G-Z (SEQ ID NO: 1277), X-ARKRERAYS*GG-C(Sx)-G-Z (SEQ ID NO: 1278), X-ARKRERAYS*H[dP]-C(Sx)-G-Z (SEQ ID NO: 1279), X-ARKRERAYS*H-C(Sx)-G-Z (SEQ ID NO: 1280), X-ARKRERAYS*I[dP]-C(Sx)-G-Z (SEQ ID NO: 1281), X-ARKRERAYS*I-C(Sx)-G-Z (SEQ ID NO: 1282), X-ARKRERAYS*K[dP]-C(Sx)-G-Z (SEQ ID NO: 1283), X-ARKRERAYS*K-C(Sx)-G-Z (SEQ ID NO: 1284), X-ARKRERAYS*L[dP]-C(Sx)-G-Z (SEQ ID NO: 1285), X-ARKRERAYS*L-C(Sx)-G-Z (SEQ ID NO: 1286), X-ARKRERAYS*N-C(Sx)-G-Z (SEQ ID NO: 1287), X-ARKRERAYS*P-C(Sx)-G-Z (SEQ ID NO: 1288), X-ARKRERAYS*Q[dP]-C(Sx)-G-Z (SEQ ID NO: 1289), X-ARKRERAYS*Q-C(Sx)-G-Z (SEQ ID NO: 1290), X-ARKRERAYS*R[dP]-C(Sx)-G-Z (SEQ ID NO: 1291), X-ARKRERAYS*R-C(Sx)-G-Z (SEQ ID NO: 1292), X-ARKRERAYS*V[dP]-C(Sx)-G-Z (SEQ ID NO: 1293), X-ARKRERAYS*V-C(Sx)-G-Z (SEQ ID NO: 1294), X-ARKRERAYS*W[dP]-C(Sx)-G-Z (SEQ ID NO: 1295), X-ARKRERAYS*W-C(Sx)-G-Z (SEQ ID NO: 1296), X-ARKRERAYSN[dP]-C(Sx)-G-Z (SEQ ID NO: 1297), X-ARKRERAYSP[dP]-C(Sx)-G-Z (SEQ ID NO: 1298), X-ARKRRRHPS*G-C(Sx)-PTA-Z (SEQ ID NO: 1299), and X-ARR-C(Sx)-AS*FRRR-Z (SEQ ID NO: 1300); wherein X— is H or acetyl; and Z is OH or NH2.

In some embodiments, the metal-binding compound of the present invention is selected from the group consisting of: X-ART*K-C(Sx)-TARKSTGGKAPRK-Z (SEQ ID NO: 1301), X-ART*K-C(Sx)-TARKSTGGK-Z (SEQ ID NO: 1302), X-ARTKQT*A-C(Sx)-KSTGGKAPRK-Z (SEQ ID NO: 1303), X-ARTKQT*A-C(Sx)-KSTGGK-Z (SEQ ID NO: 1304), X-ARTKQTARKS*T-C(Sx)-GKAPRK-Z (SEQ ID NO: 1305), X-ARTKQTARKS*T-C(Sx)-GK-Z (SEQ ID NO: 1306), X-ARVS*P-C(Sx)-ESPRARAAA-Z (SEQ ID NO: 1307), X-ARVSP-C(Sx)-ES*PRARAAA-Z (SEQ ID NO: 1308), X-ARVSPPES*P-C(Sx)-ARAAA-Z (SEQ ID NO: 1101), X-ATEEIYLT*P-C(Sx)-QRPPD-Z (SEQ ID NO: 1117), X-AVSDSLPS*N-C(Sx)-LKKSS-Z (SEQ ID NO: 1179), X-C(Sx)-[Nle]S*LRRKAK-Z (SEQ ID NO: 1309), X-C(Sx)-AS*LRRKAK-Z (SEQ ID NO: 1310), X-C(Sx)-DS*LRRKAK-Z (SEQ ID NO: 1311), X-C(Sx)-ES*LRRKAK-Z (SEQ ID NO: 1312), X-C(Sx)-ES*QEAFADLWKK-Z (SEQ ID NO: 1313), X-C(Sx)-ES*QETFSDLWKK-Z (SEQ ID NO: 1314), X-C(Sx)-FS*[Nle]RRKAK-Z (SEQ ID NO: 1315), X-C(Sx)-FS*ARRKAK-Z (SEQ ID NO: 1316), X-C(Sx)-FS*DRRKAK-Z (SEQ ID NO: 1317), X-C(Sx)-FS*ERRKAK-Z (SEQ ID NO: 1318), X-C(Sx)-FS*FRRKAK-Z (SEQ ID NO: 1319), X-C(Sx)-FS*GRRKAK-Z (SEQ ID NO: 1320), X-C(Sx)-FS*HRRKAK-Z (SEQ ID NO: 1321), X-C(Sx)-FS*IRRKAK-Z (SEQ ID NO: 1322), X-C(Sx)-FS*KRRKAK-Z (SEQ ID NO: 1323), X-C(Sx)-FS*L[Nle]RKAK-Z (SEQ ID NO: 1324), X-C(Sx)-FS*LARKAK-Z (SEQ ID NO: 1325), X-C(Sx)-FS*LDRKAK-Z (SEQ ID NO: 1326), X-C(Sx)-FS*LERKAK-Z (SEQ ID NO: 1327), X-C(Sx)-FS*LFRKAK-Z (SEQ ID NO: 1328), X-C(Sx)-FS*LGRKAK-Z (SEQ ID NO: 1329), X-C(Sx)-FS*LHRKAK-Z (SEQ ID NO: 1330), X-C(Sx)-FS*LIRKAK-Z (SEQ ID NO: 1331), X-C(Sx)-FS*LKRKAK-Z (SEQ ID NO: 1332), X-C(Sx)-FS*LLRKAK-Z (SEQ ID NO: 1333), X-C(Sx)-FS*LNRKAK-Z (SEQ ID NO: 1334), X-C(Sx)-FS*LPRGAK-Z (SEQ ID NO: 1335), X-C(Sx)-FS*LPRKAK-Z (SEQ ID NO: 1336), X-C(Sx)-FS*LQRKAK-Z (SEQ ID NO: 1337), X-C(Sx)-FS*LR[Nle]KAK-Z (SEQ ID NO: 1338), X-C(Sx)-FS*LRAKAK-Z (SEQ ID NO: 1339), X-C(Sx)-FS*LRDKAK-Z (SEQ ID NO: 1340), X-C(Sx)-FS*LREKAK-Z (SEQ ID NO: 1341), X-C(Sx)-FS*LRFKAK-Z (SEQ ID NO: 1342), X-C(Sx)-FS*LRGKAK-Z (SEQ ID NO: 1343), X-C(Sx)-FS*LRHKAK-Z (SEQ ID NO: 1344), X-C(Sx)-FS*LRIKAK-Z (SEQ ID NO: 1345), X-C(Sx)-FS*LRKKAK-Z (SEQ ID NO: 1346), X-C(Sx)-FS*LRLKAK-Z (SEQ ID NO: 1347), X-C(Sx)-FS*LRNKAK-Z (SEQ ID NO: 1348), X-C(Sx)-FS*LRPKAK-Z (SEQ ID NO: 1349), X-C(Sx)-FS*LRQKAK-Z (SEQ ID NO: 1350), X-C(Sx)-FS*LRR[Nle]AK-Z (SEQ ID NO: 1351), X-C(Sx)-FS*LRRAAK-Z (SEQ ID NO: 1352), X-C(Sx)-FS*LRRDAK-Z (SEQ ID NO: 1353), X-C(Sx)-FS*LRREAK-Z (SEQ ID NO: 1354), X-C(Sx)-FS*LRRFAK-Z (SEQ ID NO: 1355), X-C(Sx)-FS*LRRGAK-Z (SEQ ID NO: 1356), X-C(Sx)-FS*LRRHAK-Z (SEQ ID NO: 1357), X-C(Sx)-FS*LRRIAK-Z (SEQ ID NO: 1358), X-C(Sx)-FS*LRRK[Nle]K-Z (SEQ ID NO: 1359), and X-C(Sx)-FS*LRRKA[Nle]-Z (SEQ ID NO: 1360); wherein X is H or acetyl; and Z is OH or NH2.

In some embodiments, the metal-binding compound of the present invention is selected from the group consisting of: X-C(Sx)-FS*LRRKAA-Z (SEQ ID NO: 1361), X-C(Sx)-FS*LRRKAD-Z (SEQ ID NO: 1362), X-C(Sx)-FS*LRRKAE-Z (SEQ ID NO: 1363), X-C(Sx)-FS*LRRKAF-Z (SEQ ID NO: 1364), X-C(Sx)-FS*LRRKAG-Z (SEQ ID NO: 1365), X-C(Sx)-FS*LRRKAH-Z (SEQ ID NO: 1366), X-C(Sx)-FS*LRRKAI-Z (SEQ ID NO: 1367), X-C(Sx)-FS*LRRKAL-Z (SEQ ID NO: 1368), X-C(Sx)-FS*LRRKAN-Z (SEQ ID NO: 1369), X-C(Sx)-FS*LRRKAP-Z (SEQ ID NO: 1370), X-C(Sx)-FS*LRRKAQ-Z (SEQ ID NO: 1371), X-C(Sx)-FS*LRRKAR-Z (SEQ ID NO: 1372), X-C(Sx)-FS*LRRKAV-Z (SEQ ID NO: 1373), X-C(Sx)-FS*LRRKAW-Z (SEQ ID NO: 1374), X-C(Sx)-FS*LRRKDK-Z (SEQ ID NO: 1375), X-C(Sx)-FS*LRRKEK-Z (SEQ ID NO: 1376), X-C(Sx)-FS*LRRKFK-Z (SEQ ID NO: 1377), X-C(Sx)-FS*LRRKGK-Z (SEQ ID NO: 1378), X-C(Sx)-FS*LRRKHK-Z (SEQ ID NO: 1379), X-C(Sx)-FS*LRRKIK-Z (SEQ ID NO: 1380), X-C(Sx)-FS*LRRKKK-Z (SEQ ID NO: 1381), X-C(Sx)-FS*LRRKLK-Z (SEQ ID NO: 1382), X-C(Sx)-FS*LRRKNK-Z (SEQ ID NO: 1383), X-C(Sx)-FS*LRRKPK-Z (SEQ ID NO: 1384), X-C(Sx)-FS*LRRKQK-Z (SEQ ID NO: 1385), X-C(Sx)-FS*LRRKRK-Z (SEQ ID NO: 1386), X-C(Sx)-FS*LRRKVK-Z (SEQ ID NO: 1387), X-C(Sx)-FS*LRRKWK-Z (SEQ ID NO: 1388), X-C(Sx)-FS*LRRLAK-Z (SEQ ID NO: 1389), X-C(Sx)-FS*LRRNAK-Z (SEQ ID NO: 1390), X-C(Sx)-FS*LRRPAK-Z (SEQ ID NO: 1391), X-C(Sx)-FS*LRRQAK-Z (SEQ ID NO: 1392), X-C(Sx)-FS*LRRRAK-Z (SEQ ID NO: 1393), X-C(Sx)-FS*LRRVAK-Z (SEQ ID NO: 1394), X-C(Sx)-FS*LRRWAK-Z (SEQ ID NO: 1395), X-C(Sx)-FS*LRVKAK-Z (SEQ ID NO: 1396), X-C(Sx)-FS*LRWKAK-Z (SEQ ID NO: 1397), X-C(Sx)-FS*LVRKAK-Z (SEQ ID NO: 1398), X-C(Sx)-FS*LWRKAK-Z (SEQ ID NO: 1399), X-C(Sx)-FS*NRRKAK-Z (SEQ ID NO: 1400), X-C(Sx)-FS*PRRKAK-Z (SEQ ID NO: 1401), X-C(Sx)-FS*QRRKAK-Z (SEQ ID NO: 1402), X-C(Sx)-FS*RRRKAK-Z (SEQ ID NO: 1403), X-C(Sx)-FS*VRRKAK-Z (SEQ ID NO: 1404), X-C(Sx)-FS*WRRKAK-Z (SEQ ID NO: 1405), X-C(Sx)-GS*LRRKAK-Z (SEQ ID NO: 1406), X-C(Sx)-HS*LKRGNR-Z (SEQ ID NO: 1407), X-C(Sx)-HS*LKRKNR-Z (SEQ ID NO: 1408), X-C(Sx)-HS*LPRFNR-Z (SEQ ID NO: 1409), X-C(Sx)-HS*LPRKNK-Z (SEQ ID NO: 1410), X-C(Sx)-HS*LRRKAK-Z (SEQ ID NO: 1411), X-C(Sx)-IS*LRRKAK-Z (SEQ ID NO: 1412), X-C(Sx)-KS*LRRKAK-Z (SEQ ID NO: 1413), X-C(Sx)-KS*PAKEKAKSPEK-Z (SEQ ID NO: 1414), X-C(Sx)-LS*FAEG-Z (SEQ ID NO: 1415), X-C(Sx)-LS*FAEPG-Z (SEQ ID NO: 1416), X-C(Sx)-LS*FRRR-Z (SEQ ID NO: 1019), X-C(Sx)-LS*LRRKAK-Z (SEQ ID NO: 1417), X-C(Sx)-LS*QEAFADLWKK-Z (SEQ ID NO: 1418), X-C(Sx)-NS*LRRKAK-Z (SEQ ID NO: 1419), X-C(Sx)-PS*LRRKAK-Z (SEQ ID NO: 1420), X-C(Sx)-QS*LRRKAK-Z (SEQ ID NO: 1421), X-C(Sx)-QS*QEAFADLWKK-Z (SEQ ID NO: 1422), X-C(Sx)-QS*QETFS*DLWKK-Z (SEQ ID NO: 1423), and X-C(Sx)-RS*LRRKAK-Z (SEQ ID NO: 1424); wherein X— is H or acetyl; and Z is OH or NH2.

In some embodiments, the metal-binding compound of the present invention is selected from the group consisting of: X-C(Sx)-RT*KQTARKSTGGKAPRK-Z (SEQ ID NO: 1425), X-C(Sx)-RT*KQTARKSTGGK-Z (SEQ ID NO: 1426), X-C(Sx)-VS*LRRKAK-Z (SEQ ID NO: 1427), X-C(Sx)-WS*LRRKAK-Z (SEQ ID NO: 1428), X-D[Nle]NPLMA-C(Sx)-GT*LTRRHQNGRF-Z (SEQ ID NO: 1429), X-DA[Nle]PLMA-C(Sx)-GT*LTRRHQNGRF-Z (SEQ ID NO: 1430), X-DAAPLMA-C(Sx)-GT*LTRRHQNGRF-Z (SEQ ID NO: 1431), X-DADPLMA-C(Sx)-GT*LTRRHQNGRF-Z (SEQ ID NO: 1432), X-DADRSILS*P-C(Sx)-GSSGP-Z (SEQ ID NO: 1433), X-DADRSILSP-C(Sx)-GS*SGP-Z (SEQ ID NO: 1434), X-DAEPLMA-C(Sx)-GT*LTRRHQNGRF-Z (SEQ ID NO: 1435), X-DAFPLMA-C(Sx)-GT*LTRRHQNGRF-Z (SEQ ID NO: 1436), X-DAGPLMA-C(Sx)-GT*LTRRHQNGRF-Z (SEQ ID NO: 1437), X-DAHPLMA-C(Sx)-GT*LTRRHQNGRF-Z (SEQ ID NO: 1438), X-DAIPLMA-C(Sx)-GT*LTRRHQNGRF-Z (SEQ ID NO: 1439), X-DAKPLMA-C(Sx)-GT*LTRRHQNGRF-Z (SEQ ID NO: 1440), X-DAKTTKKRPQRATS*N-C(Sx)-FS-Z (SEQ ID NO: 1441), X-DAKTTKKRPQRATSN-C(Sx)-FS*-Z (SEQ ID NO: 1442), X-DALPLMA-C(Sx)-GT*LTRRHQNGRF-Z (SEQ ID NO: 1443), X-DAN[Nle]LMA-C(Sx)-GT*LTRRHQNGRF-Z (SEQ ID NO: 1444), X-DANALMA-C(Sx)-GT*LTRRHQNGRF-Z (SEQ ID NO: 1445), X-DANDLMA-C(Sx)-GT*LTRRHQNGRF-Z (SEQ ID NO: 1446), X-DANELMA-C(Sx)-GT*LTRRHQNGRF-Z (SEQ ID NO: 1447), X-DANFLMA-C(Sx)-GT*LTRRHQNGRF-Z (SEQ ID NO: 1448), X-DANGLMA-C(Sx)-GT*LTRRHQNGRF-Z (SEQ ID NO: 1449), X-DANHLMA-C(Sx)-GT*LTRRHQNGRF-Z (SEQ ID NO: 1450), X-DANILMA-C(Sx)-GT*LTRRHQNGRF-Z (SEQ ID NO: 1451), X-DANKLMA-C(Sx)-GT*LTRRHQNGRF-Z (SEQ ID NO: 1452), X-DANLLMA-C(Sx)-GT*LTRRHQNGRF-Z (SEQ ID NO: 1453), X-DANNLMA-C(Sx)-GT*LTRRHQNGRF-Z (SEQ ID NO: 1454), X-DANP[Nle]MA-C(Sx)-GT*LTRRHQNGRF-Z (SEQ ID NO: 1455), X-DANPAMA-C(Sx)-GT*LTRRHQNGRF-Z (SEQ ID NO: 1456), X-DANPDMA-C(Sx)-GT*LTRRHQNGRF-Z (SEQ ID NO: 1457), X-DANPEMA-C(Sx)-GT*LTRRHQNGRF-Z (SEQ ID NO: 1458), X-DANPFMA-C(Sx)-GT*LTRRHQNGRF-Z (SEQ ID NO: 1459), X-DANPGMA-C(Sx)-GT*LTRRHQNGRF-Z (SEQ ID NO: 1460), X-DANPHMA-C(Sx)-GT*LTRRHQNGRF-Z (SEQ ID NO: 1461), X-DANPIMA-C(Sx)-GT*LTRRHQNGRF-Z (SEQ ID NO: 1462), X-DANPKMA-C(Sx)-GT*LTRRHQNGRF-Z (SEQ ID NO: 1463), X-DANPL[Nle]A-C(Sx)-GT*LTRRHQNGRF-Z (SEQ ID NO: 1464), X-DANPLAA-C(Sx)-GT*LTRRHQNGRF-Z (SEQ ID NO: 1465), X-DANPLDA-C(Sx)-GT*LTRRHQNGRF-Z (SEQ ID NO: 1466), X-DANPLEA-C(Sx)-GT*LTRRHQNGRF-Z (SEQ ID NO: 1467), X-DANPLFA-C(Sx)-GT*LTRRHQNGRF-Z (SEQ ID NO: 1468), X-DANPLGA-C(Sx)-GT*LTRRHQNGRF-Z (SEQ ID NO: 1469), X-DANPLHA-C(Sx)-GT*LTRRHQNGRF-Z (SEQ ID NO: 1470), X-DANPLIA-C(Sx)-GT*LTRRHQNGRF-Z (SEQ ID NO: 1471), X-DANPLKA-C(Sx)-GT*LTRRHQNGRF-Z (SEQ ID NO: 1472), X-DANPLLA-C(Sx)-GT*LTRRHQNGRF-Z (SEQ ID NO: 1473), X-DANPLM[Nle]-C(Sx)-GT*LTRRHQNGRF-Z (SEQ ID NO: 1474), X-DANPLMA-C(Sx)-[Nle]T*LTRRHQNGRF-Z (SEQ ID NO: 1475), X-DANPLMA-C(Sx)-AT*LTRRHQNGRF-Z (SEQ ID NO: 1476), X-DANPLMA-C(Sx)-DT*LTRRHQNGRF-Z (SEQ ID NO: 1477), X-DANPLMA-C(Sx)-ET*LTRRHQNGRF-Z (SEQ ID NO: 1478), and X-DANPLMA-C(Sx)-FT*LTRRHQNGRF-Z (SEQ ID NO: 1479); wherein X— is H or acetyl; and Z is OH or NH2.

In some embodiments, the metal-binding compound of the present invention is selected from the group consisting of: X-DANPLMA-C(Sx)-GT*ATRRHQNGRF-Z (SEQ ID NO: 1480), X-DANPLMA-C(Sx)-GT*DTRRHQNGRF-Z (SEQ ID NO: 1481), X-DANPLMA-C(Sx)-GT*ETRRHQNGRF-Z (SEQ ID NO: 1482), X-DANPLMA-C(Sx)-GT*FTRRHQNGRF-Z (SEQ ID NO: 1483), X-DANPLMA-C(Sx)-GT*GT*RRHQNGRF-Z (SEQ ID NO: 1484), X-DANPLMA-C(Sx)-GT*HTRRHQNGRF-Z (SEQ ID NO: 1485), X-DANPLMA-C(Sx)-GT*ITRRHQNGRF-Z (SEQ ID NO: 1486), X-DANPLMA-C(Sx)-GT*KTRRHQNGRF-Z (SEQ ID NO: 1487), X-DANPLMA-C(Sx)-GT*L[Nle]RRHQNGRF-Z (SEQ ID NO: 1488), X-DANPLMA-C(Sx)-GT*LARRHQNGRF-Z (SEQ ID NO: 1202), X-DANPLMA-C(Sx)-GT*LDRRHQNGRF-Z (SEQ ID NO: 1489), X-DANPLMA-C(Sx)-GT*LERRHQNGRF-Z (SEQ ID NO: 1490), X-DANPLMA-C(Sx)-GT*LFRRHQNGRF-Z (SEQ ID NO: 1491), X-DANPLMA-C(Sx)-GT*LGRRHQNGRF-Z (SEQ ID NO: 1492), X-DANPLMA-C(Sx)-GT*LHRRHQNGRF-Z (SEQ ID NO: 1493), X-DANPLMA-C(Sx)-GT*LIRRHQNGRF-Z (SEQ ID NO: 1494), X-DANPLMA-C(Sx)-GT*LKRRHQNGRF-Z (SEQ ID NO: 1495), X-DANPLMA-C(Sx)-GT*LLRRHQNGRF-Z (SEQ ID NO: 1496), X-DANPLMA-C(Sx)-GT*LNRRHQNGRF-Z (SEQ ID NO: 1497), X-DANPLMA-C(Sx)-GT*LPRRHQNGRF-Z (SEQ ID NO: 1498), X-DANPLMA-C(Sx)-GT*LQRRHQNGRF-Z (SEQ ID NO: 1499), X-DANPLMA-C(Sx)-GT*LRRRHQNGRF-Z (SEQ ID NO: 1500), X-DANPLMA-C(Sx)-GT*LT[Nle]RHQNGRF-Z (SEQ ID NO: 1501), X-DANPLMA-C(Sx)-GT*LTARHQNGRF-Z (SEQ ID NO: 1502), X-DANPLMA-C(Sx)-GT*LTDRHQNGRF-Z (SEQ ID NO: 1503), X-DANPLMA-C(Sx)-GT*LTERHQNGRF-Z (SEQ ID NO: 1504), X-DANPLMA-C(Sx)-GT*LTFRHQNGRF-Z (SEQ ID NO: 1505), X-DANPLMA-C(Sx)-GT*LTGRHQNGRF-Z (SEQ ID NO: 1506), X-DANPLMA-C(Sx)-GT*LTHRHQNGRF-Z (SEQ ID NO: 1507), X-DANPLMA-C(Sx)-GT*LTIRHQNGRF-Z (SEQ ID NO: 1508), X-DANPLMA-C(Sx)-GT*LTKRHQNGRF-Z (SEQ ID NO: 1509), X-DANPLMA-C(Sx)-GT*LTLRHQNGRF-Z (SEQ ID NO: 1510), X-DANPLMA-C(Sx)-GT*LTNRHQNGRF-Z (SEQ ID NO: 1511), X-DANPLMA-C(Sx)-GT*LTPRHQNGRF-Z (SEQ ID NO: 1512), X-DANPLMA-C(Sx)-GT*LTQRHQNGRF-Z (SEQ ID NO: 1513), X-DANPLMA-C(Sx)-GT*LTR[Nle]HQNGRF-Z (SEQ ID NO: 1514), X-DANPLMA-C(Sx)-GT*LTRAHQNGRF-Z (SEQ ID NO: 1515), X-DANPLMA-C(Sx)-GT*LTRDHQNGRF-Z (SEQ ID NO: 1516), X-DANPLMA-C(Sx)-GT*LTREHQNGRF-Z (SEQ ID NO: 1517), X-DANPLMA-C(Sx)-GT*LTRFHQNGRF-Z (SEQ ID NO: 1518), X-DANPLMA-C(Sx)-GT*LTRGHQNGRF-Z (SEQ ID NO: 1519), X-DANPLMA-C(Sx)-GT*LTRHHQNGRF-Z (SEQ ID NO: 1520), X-DANPLMA-C(Sx)-GT*LTRIHQNGRF-Z (SEQ ID NO: 1521), X-DANPLMA-C(Sx)-GT*LTRKHQNGRF-Z (SEQ ID NO: 1522), X-DANPLMA-C(Sx)-GT*LTRLHQNGRF-Z (SEQ ID NO: 1523), X-DANPLMA-C(Sx)-GT*LTRNHQNGRF-Z (SEQ ID NO: 1524), X-DANPLMA-C(Sx)-GT*LTRPHQNGRF-Z (SEQ ID NO: 1525), X-DANPLMA-C(Sx)-GT*LTRQHQNGRF-Z (SEQ ID NO: 1526), X-DANPLMA-C(Sx)-GT*LTRR[Nle]QNGRF-Z (SEQ ID NO: 1527), X-DANPLMA-C(Sx)-GT*LTRRAQNGRF-Z (SEQ ID NO: 1528), X-DANPLMA-C(Sx)-GT*LTRRDQNGRF-Z (SEQ ID NO: 1529), X-DANPLMA-C(Sx)-GT*LTRREQNGRF-Z (SEQ ID NO: 1530), X-DANPLMA-C(Sx)-GT*LTRRFQNGRF-Z (SEQ ID NO: 1531), X-DANPLMA-C(Sx)-GT*LTRRGQNGRF-Z (SEQ ID NO: 1532), and X-DANPLMA-C(Sx)-GT*LTRRH[Nle]NGRF-Z (SEQ ID NO: 1533); wherein X is H or acetyl; and Z is OH or NH2.

In some embodiments, the metal-binding compound of the present invention is selected from the group consisting of: X-DANPLMA-C(Sx)-GT*LTRRHANGRF-Z (SEQ ID NO: 1534), X-DANPLMA-C(Sx)-GT*LTRRHDNGRF-Z (SEQ ID NO: 1535), X-DANPLMA-C(Sx)-GT*LTRRHENGRF-Z (SEQ ID NO: 1536), X-DANPLMA-C(Sx)-GT*LTRRHFNGRF-Z (SEQ ID NO: 1537), X-DANPLMA-C(Sx)-GT*LTRRHGNGRF-Z (SEQ ID NO: 1538), X-DANPLMA-C(Sx)-GT*LTRRHHNGRF-Z (SEQ ID NO: 1539), X-DANPLMA-C(Sx)-GT*LTRRHINGRF-Z (SEQ ID NO: 1540), X-DANPLMA-C(Sx)-GT*LTRRHKNGRF-Z (SEQ ID NO: 1541), X-DANPLMA-C(Sx)-GT*LTRRHLNGRF-Z (SEQ ID NO: 1542), X-DANPLMA-C(Sx)-GT*LTRRHNNGRF-Z (SEQ ID NO: 1543), X-DANPLMA-C(Sx)-GT*LTRRHPNGRF-Z (SEQ ID NO: 1544), X-DANPLMA-C(Sx)-GT*LTRRHQ[Nle]GRF-Z (SEQ ID NO: 1545), X-DANPLMA-C(Sx)-GT*LTRRHQAGRF-Z (SEQ ID NO: 1546), X-DANPLMA-C(Sx)-GT*LTRRHQDGRF-Z (SEQ ID NO: 1547), X-DANPLMA-C(Sx)-GT*LTRRHQEGRF-Z (SEQ ID NO: 1548), X-DANPLMA-C(Sx)-GT*LTRRHQFGRF-Z (SEQ ID NO: 1549), X-DANPLMA-C(Sx)-GT*LTRRHQGGRF-Z (SEQ ID NO: 1550), X-DANPLMA-C(Sx)-GT*LTRRHQHGRF-Z (SEQ ID NO: 1551), X-DANPLMA-C(Sx)-GT*LTRRHQIGRF-Z (SEQ ID NO: 1552), X-DANPLMA-C(Sx)-GT*LTRRHQKGRF-Z (SEQ ID NO: 1553), X-DANPLMA-C(Sx)-GT*LTRRHQLGRF-Z (SEQ ID NO: 1554), X-DANPLMA-C(Sx)-GT*LTRRHQN[Nle]RF-Z (SEQ ID NO: 1555), X-DANPLMA-C(Sx)-GT*LTRRHQNARF-Z (SEQ ID NO: 1556), X-DANPLMA-C(Sx)-GT*LTRRHQNDRF-Z (SEQ ID NO: 1557), X-DANPLMA-C(Sx)-GT*LTRRHQNERF-Z (SEQ ID NO: 1558), X-DANPLMA-C(Sx)-GT*LTRRHQNFRF-Z (SEQ ID NO: 1559), X-DANPLMA-C(Sx)-GT*LTRRHQNG[Nle]F-Z (SEQ ID NO: 1560), X-DANPLMA-C(Sx)-GT*LTRRHQNGAF-Z (SEQ ID NO: 1561), X-DANPLMA-C(Sx)-GT*LTRRHQNGDF-Z (SEQ ID NO: 1562), X-DANPLMA-C(Sx)-GT*LTRRHQNGEF-Z (SEQ ID NO: 1563), X-DANPLMA-C(Sx)-GT*LTRRHQNGFF-Z (SEQ ID NO: 1564), X-DANPLMA-C(Sx)-GT*LTRRHQNGGF-Z (SEQ ID NO: 1565), X-DANPLMA-C(Sx)-GT*LTRRHQNGHF-Z (SEQ ID NO: 1566), X-DANPLMA-C(Sx)-GT*LTRRHQNGIF-Z (SEQ ID NO: 1567), X-DANPLMA-C(Sx)-GT*LTRRHQNGKF-Z (SEQ ID NO: 1568), X-DANPLMA-C(Sx)-GT*LTRRHQNGLF-Z (SEQ ID NO: 1569), X-DANPLMA-C(Sx)-GT*LTRRHQNGNF-Z (SEQ ID NO: 1570), X-DANPLMA-C(Sx)-GT*LTRRHQNGPF-Z (SEQ ID NO: 1571), X-DANPLMA-C(Sx)-GT*LTRRHQNGQF-Z (SEQ ID NO: 1572), X-DANPLMA-C(Sx)-GT*LTRRHQNGR[Nle]-Z (SEQ ID NO: 1573), X-DANPLMA-C(Sx)-GT*LTRRHQNGRA-Z (SEQ ID NO: 1574), X-DANPLMA-C(Sx)-GT*LTRRHQNGRD-Z (SEQ ID NO: 1575), X-DANPLMA-C(Sx)-GT*LTRRHQNGRE-Z (SEQ ID NO: 1576), X-DANPLMA-C(Sx)-GT*LTRRHQNGRF-Z (SEQ ID NO: 1200), X-DANPLMA-C(Sx)-GT*LTRRHQNGRG-Z (SEQ ID NO: 1577), X-DANPLMA-C(Sx)-GT*LTRRHQNGRH-Z (SEQ ID NO: 1578), X-DANPLMA-C(Sx)-GT*LTRRHQNGRI-Z (SEQ ID NO: 1579), X-DANPLMA-C(Sx)-GT*LTRRHQNGRK-Z (SEQ ID NO: 1580), X-DANPLMA-C(Sx)-GT*LTRRHQNGRL-Z (SEQ ID NO: 1581), X-DANPLMA-C(Sx)-GT*LTRRHQNGRN-Z (SEQ ID NO: 1582), and X-DANPLMA-C(Sx)-GT*LTRRHQNGRP-Z (SEQ ID NO: 1583); wherein X is H or acetyl; and Z is OH or NH2.

In some embodiments, the metal-binding compound of the present invention is selected from the group consisting of: X-DANPLMA-C(Sx)-GT*LTRRHQNGRQ-Z (SEQ ID NO: 1584), X-DANPLMA-C(Sx)-GT*LTRRHQNGRR-Z (SEQ ID NO: 1585), X-DANPLMA-C(Sx)-GT*LTRRHQNGRV-Z (SEQ ID NO: 1586), X-DANPLMA-C(Sx)-GT*LTRRHQNGRW-Z (SEQ ID NO: 1587), X-DANPLMA-C(Sx)-GT*LTRRHQNGVF-Z (SEQ ID NO: 1588), X-DANPLMA-C(Sx)-GT*LTRRHQNGWF-Z (SEQ ID NO: 1589), X-DANPLMA-C(Sx)-GT*LTRRHQNHRF-Z (SEQ ID NO: 1590), X-DANPLMA-C(Sx)-GT*LTRRHQNIRF-Z (SEQ ID NO: 1591), X-DANPLMA-C(Sx)-GT*LTRRHQNKRF-Z (SEQ ID NO: 1592), X-DANPLMA-C(Sx)-GT*LTRRHQNLRF-Z (SEQ ID NO: 1593), X-DANPLMA-C(Sx)-GT*LTRRHQNNRF-Z (SEQ ID NO: 1594), X-DANPLMA-C(Sx)-GT*LTRRHQNPRF-Z (SEQ ID NO: 1595), X-DANPLMA-C(Sx)-GT*LTRRHQNQRF-Z (SEQ ID NO: 1596), X-DANPLMA-C(Sx)-GT*LTRRHQNRRF-Z (SEQ ID NO: 1597), X-DANPLMA-C(Sx)-GT*LTRRHQNVRF-Z (SEQ ID NO: 1598), X-DANPLMA-C(Sx)-GT*LTRRHQNWRF-Z (SEQ ID NO: 1599), X-DANPLMA-C(Sx)-GT*LTRRHQPGRF-Z (SEQ ID NO: 1600), X-DANPLMA-C(Sx)-GT*LTRRHQQGRF-Z (SEQ ID NO: 1601), X-DANPLMA-C(Sx)-GT*LTRRHQRGRF-Z (SEQ ID NO: 1602), X-DANPLMA-C(Sx)-GT*LTRRHQVGRF-Z (SEQ ID NO: 1603), X-DANPLMA-C(Sx)-GT*LTRRHQWGRF-Z (SEQ ID NO: 1604), X-DANPLMA-C(Sx)-GT*LTRRHRNGRF-Z (SEQ ID NO: 1605), X-DANPLMA-C(Sx)-GT*LTRRHVNGRF-Z (SEQ ID NO: 1606), X-DANPLMA-C(Sx)-GT*LTRRHWNGRF-Z (SEQ ID NO: 1607), X-DANPLMA-C(Sx)-GT*LTRRIQNGRF-Z (SEQ ID NO: 1608), X-DANPLMA-C(Sx)-GT*LTRRKQNGRF-Z (SEQ ID NO: 1609), X-DANPLMA-C(Sx)-GT*LTRRLQNGRF-Z (SEQ ID NO: 1610), X-DANPLMA-C(Sx)-GT*LTRRNQNGRF-Z (SEQ ID NO: 1611), X-DANPLMA-C(Sx)-GT*LTRRPQNGRF-Z (SEQ ID NO: 1612), X-DANPLMA-C(Sx)-GT*LTRRQQNGRF-Z (SEQ ID NO: 1613), X-DANPLMA-C(Sx)-GT*LTRRRQNGRF-Z (SEQ ID NO: 1614), X-DANPLMA-C(Sx)-GT*LTRRVQNGRF-Z (SEQ ID NO: 1615), X-DANPLMA-C(Sx)-GT*LTRRWQNGRF-Z (SEQ ID NO: 1616), X-DANPLMA-C(Sx)-GT*LTRVHQNGRF-Z (SEQ ID NO: 1617), X-DANPLMA-C(Sx)-GT*LTRWHQNGRF-Z (SEQ ID NO: 1618), X-DANPLMA-C(Sx)-GT*LTVRHQNGRF-Z (SEQ ID NO: 1619), X-DANPLMA-C(Sx)-GT*LTWRHQNGRF-Z (SEQ ID NO: 1620), X-DANPLMA-C(Sx)-GT*LVRRHQNGRF-Z (SEQ ID NO: 1621), X-DANPLMA-C(Sx)-GT*LWRRHQNGRF-Z (SEQ ID NO: 1622), X-DANPLMA-C(Sx)-GT*NTRRHQNGRF-Z (SEQ ID NO: 1623), X-DANPLMA-C(Sx)-GT*PTRRHQNGRF-Z (SEQ ID NO: 1624), X-DANPLMA-C(Sx)-GT*QTRRHQNGRF-Z (SEQ ID NO: 1625), X-DANPLMA-C(Sx)-GT*RTRRHQNGRF-Z (SEQ ID NO: 1626), X-DANPLMA-C(Sx)-GT*VTRRHQNGRF-Z (SEQ ID NO: 1627), X-DANPLMA-C(Sx)-GT*WTRRHQNGRF-Z (SEQ ID NO: 1628), X-DANPLMA-C(Sx)-GT[Nle]TRRHQNGRF-Z (SEQ ID NO: 1629), X-DANPLMA-C(Sx)-HT*LTRRHQNGRF-Z (SEQ ID NO: 1630), and X-DANPLMA-C(Sx)-IT*LTRRHQNGRF-Z (SEQ ID NO: 1631); wherein X is H or acetyl; and Z is OH or NH2.

In some embodiments, the metal-binding compound of the present invention is selected from the group consisting of: X-DANPLMA-C(Sx)-KT*LTRRHQNGRF-Z (SEQ ID NO: 1632), X-DANPLMA-C(Sx)-LT*LTRRHQNGRF-Z (SEQ ID NO: 1633), X-DANPLMA-C(Sx)-NT*LTRRHQNGRF-Z (SEQ ID NO: 1634), X-DANPLMA-C(Sx)-PT*LTRRHQNGRF-Z (SEQ ID NO: 1635), X-DANPLMA-C(Sx)-QT*LTRRHQNGRF-Z (SEQ ID NO: 1636), X-DANPLMA-C(Sx)-RT*LTRRHQNGRF-Z (SEQ ID NO: 1637), X-DANPLMA-C(Sx)-VT*LTRRHQNGRF-Z (SEQ ID NO: 1638), X-DANPLMA-C(Sx)-WT*LTRRHQNGRF-Z (SEQ ID NO: 1639), X-DANPLMANGT*L-C(Sx)-RRHQNGRF-Z (SEQ ID NO: 1199), X-DANPLM-C(Sx)-NGT*LARRHQNGRF-Z (SEQ ID NO: 1203), X-DANPLM-C(Sx)-NGT*LTRRHQNGRF-Z (SEQ ID NO: 1201), X-DANPLMD-C(Sx)-GT*LTRRHQNGRF-Z (SEQ ID NO: 1640), X-DANPLME-C(Sx)-GT*LTRRHQNGRF-Z (SEQ ID NO: 1641), X-DANPLMF-C(Sx)-GT*LTRRHQNGRF-Z (SEQ ID NO: 1642), X-DANPLMG-C(Sx)-GT*LTRRHQNGRF-Z (SEQ ID NO: 1643), X-DANPLMH-C(Sx)-GT*LTRRHQNGRF-Z (SEQ ID NO: 1644), X-DANPLMI-C(Sx)-GT*LTRRHQNGRF-Z (SEQ ID NO: 1645), X-DANPLMK-C(Sx)-GT*LTRRHQNGRF-Z (SEQ ID NO: 1646), X-DANPLML-C(Sx)-GT*LTRRHQNGRF-Z (SEQ ID NO: 1647), X-DANPLMN-C(Sx)-GT*LTRRHQNGRF-Z (SEQ ID NO: 1648), X-DANPLMP-C(Sx)-GT*LTRRHQNGRF-Z (SEQ ID NO: 1649), X-DANPLMQ-C(Sx)-GT*LTRRHQNGRF-Z (SEQ ID NO: 1650), X-DANPLMR-C(Sx)-GT*LTRRHQNGRF-Z (SEQ ID NO: 1651), X-DANPLMV-C(Sx)-GT*LTRRHQNGRF-Z (SEQ ID NO: 1652), X-DANPLMW-C(Sx)-GT*LTRRHQNGRF-Z (SEQ ID NO: 1653), X-DANPLNA-C(Sx)-GT*LTRRHQNGRF-Z (SEQ ID NO: 1654), X-DANPLPA-C(Sx)-GT*LTRRHQNGRF-Z (SEQ ID NO: 1655), X-DANPLQA-C(Sx)-GT*LTRRHQNGRF-Z (SEQ ID NO: 1656), X-DANPLRA-C(Sx)-GT*LTRRHQNGRF-Z (SEQ ID NO: 1657), X-DANPLVA-C(Sx)-GT*LTRRHQNGRF-Z (SEQ ID NO: 1658), X-DANPLWA-C(Sx)-GT*LTRRHQNGRF-Z (SEQ ID NO: 1659), X-DANPNMA-C(Sx)-GT*LTRRHQNGRF-Z (SEQ ID NO: 1660), X-DANPPMA-C(Sx)-GT*LTRRHQNGRF-Z (SEQ ID NO: 1661), X-DANPQMA-C(Sx)-GT*LTRRHQNGRF-Z (SEQ ID NO: 1662), X-DANPRMA-C(Sx)-GT*LTRRHQNGRF-Z (SEQ ID NO: 1663), X-DANPVMA-C(Sx)-GT*LTRRHQNGRF-Z (SEQ ID NO: 1664), X-DANPWMA-C(Sx)-GT*LTRRHQNGRF-Z (SEQ ID NO: 1665), X-DANQLMA-C(Sx)-GT*LTRRHQNGRF-Z (SEQ ID NO: 1666), X-DANRLMA-C(Sx)-GT*LTRRHQNGRF-Z (SEQ ID NO: 1667), X-DANVLMA-C(Sx)-GT*LTRRHQNGRF-Z (SEQ ID NO: 1668), X-DANWLMA-C(Sx)-GT*LTRRHQNGRF-Z (SEQ ID NO: 1669), X-DAPPLMA-C(Sx)-GT*LTRRHQNGRF-Z (SEQ ID NO: 1670), X-DAQPLMA-C(Sx)-GT*LTRRHQNGRF-Z (SEQ ID NO: 1671), X-DARPLMA-C(Sx)-GT*LTRRHQNGRF-Z (SEQ ID NO: 1672), X-DAVPLMA-C(Sx)-GT*LTRRHQNGRF-Z (SEQ ID NO: 1673), X-DAWPLMA-C(Sx)-GT*LTRRHQNGRF-Z (SEQ ID NO: 1674), X-D-C(Sx)-DS*LRRKAK-Z (SEQ ID NO: 1675), X-D-C(Sx)-LS*PGPF-Z (SEQ ID NO: 1676), X-DDIEQWFTE-C(Sx)-PGPDE-Z (SEQ ID NO: 1677), X-DDNPLMA-C(Sx)-GT*LTRRHQNGRF-Z (SEQ ID NO: 1678), X-DE-C(Sx)-EGT*FRAAIRRLAARRR-Z (SEQ ID NO: 665), X-DE-C(Sx)-EGT*FRSSIRRLSSRRR-Z (SEQ ID NO: 662), and X-DENPLMA-C(Sx)-GT*LTRRHQNGRF-Z (SEQ ID NO: 1679); wherein X— is H or acetyl; and Z is OH or NH2.

In some embodiments, the metal-binding compound of the present invention is selected from the group consisting of: X-DFGLSKWR[Nle][Nle]S*L-C(Sx)-QSRSSKS-Z (SEQ ID NO: 1680), X-DFGLSKWR[Nle][Nle]SL-C(Sx)-QS*RSSKS-Z (SEQ ID NO: 1681), X-DFGLSKWRMMS*L-C(Sx)-QSRSSKS-Z (SEQ ID NO: 1682), X-DFGLSKWRMMS*L-C(Sx)-QSR-Z (SEQ ID NO: 1683), X-DFGLSKWRMMS*L-C(Sx)-Q-Z (SEQ ID NO: 1684), X-DFGLSKWRMMSL-C(Sx)-QS*RSSKS-Z (SEQ ID NO: 1685), X-DFGLSKWRMMSL-C(Sx)-QS*R-Z (SEQ ID NO: 1686), X-DFNPLMA-C(Sx)-GT*LTRRHQNGRF-Z (SEQ ID NO: 1687), X-DGNPLMA-C(Sx)-GT*LTRRHQNGRF-Z (SEQ ID NO: 1688), X-DHNPLMA-C(Sx)-GT*LTRRHQNGRF-Z (SEQ ID NO: 1689), X-DINPLMA-C(Sx)-GT*LTRRHQNGRF-Z (SEQ ID NO: 1690), X-DKNPLMA-C(Sx)-GT*LTRRHQNGRF-Z (SEQ ID NO: 1691), X-DKRPQRAT*S-C(Sx)-VFAMF-Z (SEQ ID NO: 1692), X-DLGYAKDV-C(Sx)-QGS*LCTSFVGTLQYLAPEKKK-Z (SEQ ID NO: 1013), X-DLGYAKDVDQGS*L-C(Sx)-TSFVGTLQYLAPE-Z (SEQ ID NO: 1014), X-DLGYAKDVDQGSL-C(Sx)-TS*FVGTLQYLAPE-Z (SEQ ID NO: 1015), X-DLGYAKDVDQGSLCTS*F-C(Sx)-GTLQYLAPE-Z (SEQ ID NO: 1016), X-DLGYAKDVDQGSLCTSF-C(Sx)-GT*LQYLAPE-Z (SEQ ID NO: 1693), X-DLI[Nle]KEDYE-C(Sx)-VS*TKPTR-Z (SEQ ID NO: 1694), X-DLIMKEDYE-C(Sx)-VS*TKPTR-Z (SEQ ID NO: 1695), X-DLLR-C(Sx)-SS*RRIRR-Z (SEQ ID NO: 1696), X-DLNPLMA-C(Sx)-GT*LTRRHQNGRF-Z (SEQ ID NO: 1697), X-DLPDM-C(Sx)-ET*KYTVDKRFGM-Z (SEQ ID NO: 1698), X-DLPDMKET*K-C(Sx)-TVDKRFGM-Z (SEQ ID NO: 1699), X-DLPDMKET-C(Sx)-YT*VDKRFGM-Z (SEQ ID NO: 1700), X-DLPDMKETKYT*V-C(Sx)-KRFGM-Z (SEQ ID NO: 1701), X-DLPWWR-C(Sx)-YT*WVVERDVNTKQR-Z (SEQ ID NO: 1702), X-DNNPLMA-C(Sx)-GT*LTRRHQNGRF-Z (SEQ ID NO: 1703), X-DPLPP-C(Sx)-FS*GTPKGSGKKK-Z (SEQ ID NO: 1704), X-DPNPLMA-C(Sx)-GT*LTRRHQNGRF-Z (SEQ ID NO: 1705), X-DPQLPSRT*P-C(Sx)-QGPRP-Z (SEQ ID NO: 1188), X-DPRGD-C(Sx)-HS*PIVLGRP-Z (SEQ ID NO: 1144), X-DPRGDFHS*P-C(Sx)-VLGRP-Z (SEQ ID NO: 1143), X-DQNPLMA-C(Sx)-GT*LTRRHQNGRF-Z (SEQ ID NO: 1706), X-DQREGFGRQS*M-C(Sx)-EKR-Z (SEQ ID NO: 1707), X-DRNPLMA-C(Sx)-GT*LTRRHQNGRF-Z (SEQ ID NO: 1708), X-DRR-C(Sx)-AS*FRRR-Z (SEQ ID NO: 1709), X-DRVS*R-C(Sx)-SSPLKTGEQ-Z (SEQ ID NO: 1710), X-DRVSR-C(Sx)-SS*PLKTGEQ-Z (SEQ ID NO: 1711), X-DSLSYYHS*P-C(Sx)-DSFSS-Z (SEQ ID NO: 1174), X-DSLSYYHSP-C(Sx)-DS*FSS-Z (SEQ ID NO: 1712), X-DVNPLMA-C(Sx)-GT*LTRRHQNGRF-Z (SEQ ID NO: 1713), and X-DWNPLMA-C(Sx)-GT*LTRRHQNGRF-Z (SEQ ID NO: 1714); wherein X is H or acetyl; and Z is OH or NH2.

In some embodiments, the metal-binding compound of the present invention is selected from the group consisting of: X-EAKTTKKRPQRATS*N-C(Sx)-FS-Z (SEQ ID NO: 1715), X-EAKTTKKRPQRATSN-C(Sx)-FS*-Z (SEQ ID NO: 1716), X-EANPLMA-C(Sx)-GT*LTRRHQNGRF-Z (SEQ ID NO: 1717), X-E-C(Sx)-DS*LRRKAK-Z (SEQ ID NO: 1718), X-E-C(Sx)-LS*PGPF-Z (SEQ ID NO: 1719), X-EDFSS*I-C(Sx)-DMDFSALLSQISS-Z (SEQ ID NO: 1720), X-EDPDYE-C(Sx)-PS*A-Z (SEQ ID NO: 1721), X-EENV-C(Sx)-DGS*PNAGSVE-Z (SEQ ID NO: 1039), X-EENVSDGS*P-C(Sx)-AGSVE-Z (SEQ ID NO: 1038), X-EEYET-C(Sx)-ES*PVPPARS-Z (SEQ ID NO: 1047), X-EEYETPES*P-C(Sx)-PPARS-Z (SEQ ID NO: 1046), X-EGMI-C(Sx)-LS*ESSRRRIRSGNKL-Z (SEQ ID NO: 1722), X-EGMILLS*E-C(Sx)-SRRRIRSGNKL-Z (SEQ ID NO: 1723), X-EIRSRHSS*Y-C(Sx)-AGTED-Z (SEQ ID NO: 1103), X-EKGPEGVS*P-C(Sx)-QAHLH-Z (SEQ ID NO: 1185), X-EKRPQRAT*S-C(Sx)-VFAMF-Z (SEQ ID NO: 1724), X-ELLR-C(Sx)-SS*RRIRR-Z (SEQ ID NO: 1725), X-ELPWWR-C(Sx)-YT*WVVERDVNTKQR-Z (SEQ ID NO: 1726), X-ELRKT-C(Sx)-TS*[Nle]SLGTTREKTD-Z (SEQ ID NO: 1727), X-ELRKTQTS[Nle]S*L-C(Sx)-TTREKTD-Z (SEQ ID NO: 1728), X-ELRKTQTS[Nle]SL-C(Sx)-TT*REKTD-Z (SEQ ID NO: 1729), X-EPAA-C(Sx)-IS*PLGALQHGFFR-Z (SEQ ID NO: 1730), X-EPAARIS*P-C(Sx)-GALQHGFFR-Z (SEQ ID NO: 1731), X-EP-C(Sx)-LS*QEAFADLWKK-Z (SEQ ID NO: 1732), X-EP-C(Sx)-LS*QETFSDLWKK-Z (SEQ ID NO: 1733), X-EP-C(Sx)-QS*QEAFADLWKK-Z (SEQ ID NO: 1734), X-EP-C(Sx)-QS*QETFSDLWK-Z (SEQ ID NO: 1735), X-EPGPVPSS*P-C(Sx)-QEPPT-Z (SEQ ID NO: 1166), X-EPIQEANYV-C(Sx)-MT*PGT-Z (SEQ ID NO: 1736), X-EPPPR-C(Sx)-KT*PEIFRAL-Z (SEQ ID NO: 1737), X-EPPPRPKT*P-C(Sx)-IFRAL-Z (SEQ ID NO: 1100), X-EQREGFGRQS*M-C(Sx)-EKR-Z (SEQ ID NO: 1738), X-ERR-C(Sx)-AS*FRRR-Z (SEQ ID NO: 1739), and X-ESPEKVS*P-C(Sx)-KISDF-Z (SEQ ID NO: 1740); wherein X— is H or acetyl; and Z is OH or NH2.

In some embodiments, the metal-binding compound of the present invention is selected from the group consisting of: X-ET-C(Sx)-ST*QELYSIPEDQE-Z (SEQ ID NO: 1741), X-EVPDVTAT*P-C(Sx)-RLLFF-Z (SEQ ID NO: 1080), X-EYSLPALT*P-C(Sx)-LDEVK-Z (SEQ ID NO: 1123), X-EYSLPALT*PG-C(Sx)-DEVK-Z (SEQ ID NO: 1742), X-EYSLP-C(Sx)-LT*PGLDEVK-Z (SEQ ID NO: 1743), X-F[Nle]REGFGRQS*M-C(Sx)-EKR-Z (SEQ ID NO: 1744), X-FAKTTKKRPQRATS*N-C(Sx)-FS-Z (SEQ ID NO: 1745), X-FAKTTKKRPQRATSN-C(Sx)-FS*-Z (SEQ ID NO: 1746), X-FANPLMA-C(Sx)-GT*LTRRHQNGRF-Z (SEQ ID NO: 1747), X-FAREGFGRQS*M-C(Sx)-EKR-Z (SEQ ID NO: 1748), X-F-C(Sx)-DS*LRRKAK-Z (SEQ ID NO: 1749), X-F-C(Sx)-LS*PGPF-Z (SEQ ID NO: 1750), X-FDREGFGRQS*M-C(Sx)-EKR-Z (SEQ ID NO: 1751), X-FEREGFGRQS*M-C(Sx)-EKR-Z (SEQ ID NO: 1752), X-FFKK-C(Sx)-VT*PRTP-Z (SEQ ID NO: 1753), X-FFKKIVT*P-C(Sx)-TP-Z (SEQ ID NO: 1754), X-FFKNIVT-C(Sx)-RT*PPPSQGK-Z (SEQ ID NO: 1755), X-FFREGFGRQS*M-C(Sx)-EKR-Z (SEQ ID NO: 1756), X-FGESPPLS*P-C(Sx)-DMDTQ-Z (SEQ ID NO: 1079), X-FGREGFGRQS*M-C(Sx)-EKR-Z (SEQ ID NO: 1757), X-FHREGFGRQS*M-C(Sx)-EKR-Z (SEQ ID NO: 1758), X-FIREGFGRQS*M-C(Sx)-EKR-Z (SEQ ID NO: 1759), X-FKREGFGRQS*M-C(Sx)-EKR-Z (SEQ ID NO: 1760), X-FKRPQRAT*S-C(Sx)-VFAMF-Z (SEQ ID NO: 1761), X-FKTEG-C(Sx)-DS*D-Z (SEQ ID NO: 1762), X-FKTEGPDS*D-C(Sx)-Z (SEQ ID NO: 1763), X-FLLR-C(Sx)-SS*RRIRR-Z (SEQ ID NO: 1764), X-FLPWWR-C(Sx)-YT*WVVERDVNTKQR-Z (SEQ ID NO: 1765), X-FLREGFGRQS*M-C(Sx)-EKR-Z (SEQ ID NO: 1766), X-FNREGFGRQS*M-C(Sx)-EKR-Z (SEQ ID NO: 1767), X-FP-C(Sx)-FS*YSASGTA-Z (SEQ ID NO: 1768), X-FPREGFGRQS*M-C(Sx)-EKR-Z (SEQ ID NO: 1769), X-FPRGSSSRST*F-C(Sx)-GEGLR-Z (SEQ ID NO: 1770), X-FQ[Nle]EGFGRQS*M-C(Sx)-EKR-Z (SEQ ID NO: 1771), X-FQAEGFGRQS*M-C(Sx)-EKR-Z (SEQ ID NO: 1772), X-FQDEGFGRQS*M-C(Sx)-EKR-Z (SEQ ID NO: 1773), X-FQEEGFGRQS*M-C(Sx)-EKR-Z (SEQ ID NO: 1774), X-FQFEGFGRQS*M-C(Sx)-EKR-Z (SEQ ID NO: 1775), X-FQGEGFGRQS*M-C(Sx)-EKR-Z (SEQ ID NO: 1776), X-FQHEGFGRQS*M-C(Sx)-EKR-Z (SEQ ID NO: 1777), X-FQIEGFGRQS*M-C(Sx)-EKR-Z (SEQ ID NO: 1778), X-FQKEGFGRQS*M-C(Sx)-EKR-Z (SEQ ID NO: 1779), X-FQLEGFGRQS*M-C(Sx)-EKR-Z (SEQ ID NO: 1780), X-FQNEGFGRQS*M-C(Sx)-EKR-Z (SEQ ID NO: 1781), X-FQPEGFGRQS*M-C(Sx)-EKR-Z (SEQ ID NO: 1782), X-FQQEGFGRQS*M-C(Sx)-EKR-Z (SEQ ID NO: 1783), X-FQR[Nle]GFGRQS*M-C(Sx)-EKR-Z (SEQ ID NO: 1784), X-FQRAGFGRQS*M-C(Sx)-EKR-Z (SEQ ID NO: 1785), X-FQRDGFGRQS*M-C(Sx)-EKR-Z (SEQ ID NO: 1786), X-FQRE[Nle]FGRQS*M-C(Sx)-EKR-Z (SEQ ID NO: 1787), X-FQREAFGRQS*M-C(Sx)-EKR-Z (SEQ ID NO: 1788), X-FQREDFGRQS*M-C(Sx)-EKR-Z (SEQ ID NO: 1789), X-FQREEFGRQS*M-C(Sx)-EKR-Z (SEQ ID NO: 1790), X-FQREFFGRQS*M-C(Sx)-EKR-Z (SEQ ID NO: 1791), X-FQREG[Nle]GRQS*M-C(Sx)-EKR-Z (SEQ ID NO: 1792), X-FQREGAGRQS*M-C(Sx)-EKR-Z (SEQ ID NO: 1793), X-FQREGDGRQS*M-C(Sx)-EKR-Z (SEQ ID NO: 1794), X-FQREGEGRQS*M-C(Sx)-EKR-Z (SEQ ID NO: 1795), X-FQREGF[Nle]RQS*M-C(Sx)-EKR-Z (SEQ ID NO: 1796), X-FQREGFARQS*M-C(Sx)-EKR-Z (SEQ ID NO: 1797), and X-FQREGFDRQS*M-C(Sx)-EKR-Z (SEQ ID NO: 1798); wherein X is H or acetyl; and Z is OH or NH2.

In some embodiments, the metal-binding compound of the present invention is selected from the group consisting of: X-FQREGFERQS*M-C(Sx)-EKR-Z (SEQ ID NO: 1799), X-FQREGFFRQS*M-C(Sx)-EKR-Z (SEQ ID NO: 1800), X-FQREGFG[Nle]QS*M-C(Sx)-EKR-Z (SEQ ID NO: 1801), X-FQREGFGAQS*M-C(Sx)-EKR-Z (SEQ ID NO: 1802), X-FQREGFGDQS*M-C(Sx)-EKR-Z (SEQ ID NO: 1803), X-FQREGFGEQS*M-C(Sx)-EKR-Z (SEQ ID NO: 1804), X-FQREGFGFQS*M-C(Sx)-EKR-Z (SEQ ID NO: 1805), X-FQREGFGGQS*M-C(Sx)-EKR-Z (SEQ ID NO: 1806), X-FQREGFGHQS*M-C(Sx)-EKR-Z (SEQ ID NO: 1807), X-FQREGFGIQS*M-C(Sx)-EKR-Z (SEQ ID NO: 1808), X-FQREGFGKQS*M-C(Sx)-EKR-Z (SEQ ID NO: 1809), X-FQREGFGLQS*M-C(Sx)-EKR-Z (SEQ ID NO: 1810), X-FQREGFGNQS*M-C(Sx)-EKR-Z (SEQ ID NO: 1811), X-FQREGFGPQS*M-C(Sx)-EKR-Z (SEQ ID NO: 1812), X-FQREGFGQQS*M-C(Sx)-EKR-Z (SEQ ID NO: 1813), X-FQREGFGR[Nle]S*M-C(Sx)-EKR-Z (SEQ ID NO: 1814), X-FQREGFGRAS*M-C(Sx)-EKR-Z (SEQ ID NO: 1815), X-FQREGFGRDS*M-C(Sx)-EKR-Z (SEQ ID NO: 1816), X-FQREGFGRES*M-C(Sx)-EKR-Z (SEQ ID NO: 1817), X-FQREGFGRFS*M-C(Sx)-EKR-Z (SEQ ID NO: 1818), X-FQREGFGRGS*M-C(Sx)-EKR-Z (SEQ ID NO: 1819), X-FQREGFGRHS*M-C(Sx)-EKR-Z (SEQ ID NO: 1820), X-FQREGFGRIS*M-C(Sx)-EKR-Z (SEQ ID NO: 1821), X-FQREGFGRKS*M-C(Sx)-EKR-Z (SEQ ID NO: 1822), X-FQREGFGRLS*M-C(Sx)-EKR-Z (SEQ ID NO: 1823), X-FQREGFGRNS*M-C(Sx)-EKR-Z (SEQ ID NO: 1824), X-FQREGFGRPS*M-C(Sx)-EKR-Z (SEQ ID NO: 1825), X-FQREGFGRQS*[Nle]-C(Sx)-EKR-Z (SEQ ID NO: 1826), X-FQREGFGRQS*A-C(Sx)-EKR-Z (SEQ ID NO: 1827), X-FQREGFGRQS*D-C(Sx)-EKR-Z (SEQ ID NO: 1828), X-FQREGFGRQS*E-C(Sx)-EKR-Z (SEQ ID NO: 1829), X-FQREGFGRQS*F-C(Sx)-EKR-Z (SEQ ID NO: 1830), X-FQREGFGRQS*G-C(Sx)-EKR-Z (SEQ ID NO: 1831), X-FQREGFGRQS*H-C(Sx)-EKR-Z (SEQ ID NO: 1832), X-FQREGFGRQS*I-C(Sx)-EKR-Z (SEQ ID NO: 1833), X-FQREGFGRQS*K-C(Sx)-EKR-Z (SEQ ID NO: 1834), X-FQREGFGRQS*L-C(Sx)-EKR-Z (SEQ ID NO: 1835), X-FQREGFGRQS*M-C(Sx)-[Nle]KR-Z (SEQ ID NO: 1836), X-FQREGFGRQS*M-C(Sx)-AKR-Z (SEQ ID NO: 1837), X-FQREGFGRQS*M-C(Sx)-DKR-Z (SEQ ID NO: 1838), X-FQREGFGRQS*M-C(Sx)-E[Nle]R-Z (SEQ ID NO: 1839), X-FQREGFGRQS*M-C(Sx)-EAATK-Z (SEQ ID NO: 1840), X-FQREGFGRQS*M-C(Sx)-EAR-Z (SEQ ID NO: 1841), X-FQREGFGRQS*M-C(Sx)-EDR-Z (SEQ ID NO: 1842), X-FQREGFGRQS*M-C(Sx)-EER-Z (SEQ ID NO: 1843), X-FQREGFGRQS*M-C(Sx)-EFR-Z (SEQ ID NO: 1844), X-FQREGFGRQS*M-C(Sx)-EGR-Z (SEQ ID NO: 1845), X-FQREGFGRQS*M-C(Sx)-EHR-Z (SEQ ID NO: 1846), X-FQREGFGRQS*M-C(Sx)-EIR-Z (SEQ ID NO: 1847), X-FQREGFGRQS*M-C(Sx)-EK[Nle]amide-Z (SEQ ID NO: 1848), X-FQREGFGRQS*M-C(Sx)-EKA-Z (SEQ ID NO: 1849), X-FQREGFGRQS*M-C(Sx)-EKD-Z (SEQ ID NO: 1850), X-FQREGFGRQS*M-C(Sx)-EKE-Z (SEQ ID NO: 1851), X-FQREGFGRQS*M-C(Sx)-EKF-Z (SEQ ID NO: 1852), X-FQREGFGRQS*M-C(Sx)-EKG-Z (SEQ ID NO: 1853), X-FQREGFGRQS*M-C(Sx)-EKH-Z (SEQ ID NO: 1854), and X-FQREGFGRQS*M-C(Sx)-EKI-Z (SEQ ID NO: 1855); wherein X is H or acetyl; and Z is OH or NH2.

In some embodiments, the metal-binding compound of the present invention is selected from the group consisting of: X-FQREGFGRQS*M-C(Sx)-EKK-Z (SEQ ID NO: 1856), X-FQREGFGRQS*M-C(Sx)-EKL-Z (SEQ ID NO: 1857), X-FQREGFGRQS*M-C(Sx)-EKN-Z (SEQ ID NO: 1858), X-FQREGFGRQS*M-C(Sx)-EKP-Z (SEQ ID NO: 1859), X-FQREGFGRQS*M-C(Sx)-EKQ-Z (SEQ ID NO: 1860), X-FQREGFGRQS*M-C(Sx)-EKR[pT]K-Z (SEQ ID NO: 1861), X-FQREGFGRQS*M-C(Sx)-EKRTK-Z (SEQ ID NO: 1862), X-FQREGFGRQS*M-C(Sx)-EKV-Z (SEQ ID NO: 1863), X-FQREGFGRQS*M-C(Sx)-EKW-Z (SEQ ID NO: 1864), X-FQREGFGRQS*M-C(Sx)-ELR-Z (SEQ ID NO: 1865), X-FQREGFGRQS*M-C(Sx)-ENR-Z (SEQ ID NO: 1866), X-FQREGFGRQS*M-C(Sx)-EPR-Z (SEQ ID NO: 1867), X-FQREGFGRQS*M-C(Sx)-EQR-Z (SEQ ID NO: 1868), X-FQREGFGRQS*M-C(Sx)-ERR-Z (SEQ ID NO: 1869), X-FQREGFGRQS*M-C(Sx)-EVR-Z (SEQ ID NO: 1870), X-FQREGFGRQS*M-C(Sx)-EWR-Z (SEQ ID NO: 1871), X-FQREGFGRQS*M-C(Sx)-FKR-Z (SEQ ID NO: 1872), X-FQREGFGRQS*M-C(Sx)-GKR-Z (SEQ ID NO: 1873), X-FQREGFGRQS*M-C(Sx)-HKR-Z (SEQ ID NO: 1874), X-FQREGFGRQS*M-C(Sx)-IKR-Z (SEQ ID NO: 1875), X-FQREGFGRQS*M-C(Sx)-KKR-Z (SEQ ID NO: 1876), X-FQREGFGRQS*M-C(Sx)-LKR-Z (SEQ ID NO: 1877), X-FQREGFGRQS*M-C(Sx)-NKR-Z (SEQ ID NO: 1878), X-FQREGFGRQS*M-C(Sx)-PKR-Z (SEQ ID NO: 1879), X-FQREGFGRQS*M-C(Sx)-QKR-Z (SEQ ID NO: 1880), X-FQREGFGRQS*M-C(Sx)-RKR-Z (SEQ ID NO: 1881), X-FQREGFGRQS*M-C(Sx)-VKR-Z (SEQ ID NO: 1882), X-FQREGFGRQS*M-C(Sx)-WKR-Z (SEQ ID NO: 1883), X-FQREGFGRQS*N-C(Sx)-EKR-Z (SEQ ID NO: 1884), X-FQREGFGRQS*P-C(Sx)-EKR-Z (SEQ ID NO: 1885), X-FQREGFGRQS*Q-C(Sx)-EKR-Z (SEQ ID NO: 1886), X-FQREGFGRQS*R-C(Sx)-EKR-Z (SEQ ID NO: 1887), X-FQREGFGRQS*V-C(Sx)-EKR-Z (SEQ ID NO: 1888), X-FQREGFGRQS*W-C(Sx)-EKR-Z (SEQ ID NO: 1889), X-FQREGFGRRS*M-C(Sx)-EKR-Z (SEQ ID NO: 1890), X-FQREGFGRVS*M-C(Sx)-EKR-Z (SEQ ID NO: 1891), X-FQREGFGRWS*M-C(Sx)-EKR-Z (SEQ ID NO: 1892), X-FQREGFGVQS*M-C(Sx)-EKR-Z (SEQ ID NO: 1893), X-FQREGFGWQS*M-C(Sx)-EKR-Z (SEQ ID NO: 1894), X-FQREGFHRQS*M-C(Sx)-EKR-Z (SEQ ID NO: 1895), X-FQREGFIRQS*M-C(Sx)-EKR-Z (SEQ ID NO: 1896), X-FQREGFKRQS*M-C(Sx)-EKR-Z (SEQ ID NO: 1897), X-FQREGFLRQS*M-C(Sx)-EKR-Z (SEQ ID NO: 1898), X-FQREGFNRQS*M-C(Sx)-EKR-Z (SEQ ID NO: 1899), X-FQREGFPRQS*M-C(Sx)-EKR-Z (SEQ ID NO: 1900), X-FQREGFQRQS*M-C(Sx)-EKR-Z (SEQ ID NO: 1901), X-FQREGFRRQS*M-C(Sx)-EKR-Z (SEQ ID NO: 1902), X-FQREGFVRQS*M-C(Sx)-EKR-Z (SEQ ID NO: 1903), X-FQREGFWRQS*M-C(Sx)-EKR-Z (SEQ ID NO: 1904), X-FQREGGGRQS*M-C(Sx)-EKR-Z (SEQ ID NO: 1905), X-FQREGHGRQS*M-C(Sx)-EKR-Z (SEQ ID NO: 1906), X-FQREGIGRQS*M-C(Sx)-EKR-Z (SEQ ID NO: 1907), X-FQREGKGRQS*M-C(Sx)-EKR-Z (SEQ ID NO: 1908), X-FQREGLGRQS*M-C(Sx)-EKR-Z (SEQ ID NO: 1909), X-FQREGNGRQS*M-C(Sx)-EKR-Z (SEQ ID NO: 1910), X-FQREGPGRQS*M-C(Sx)-EKR-Z (SEQ ID NO: 1911), X-FQREGQGRQS*M-C(Sx)-EKR-Z (SEQ ID NO: 1912), X-FQREGRGRQS*M-C(Sx)-EKR-Z (SEQ ID NO: 1913), X-FQREGVGRQS*M-C(Sx)-EKR-Z (SEQ ID NO: 1914), X-FQREGWGRQS*M-C(Sx)-EKR-Z (SEQ ID NO: 1915), X-FQREHFGRQS*M-C(Sx)-EKR-Z (SEQ ID NO: 1916), X-FQREIFGRQS*M-C(Sx)-EKR-Z (SEQ ID NO: 1917), X-FQREKFGRQS*M-C(Sx)-EKR-Z (SEQ ID NO: 1918), X-FQRELFGRQS*M-C(Sx)-EKR-Z (SEQ ID NO: 1919), X-FQRENFGRQS*M-C(Sx)-EKR-Z (SEQ ID NO: 1920), X-FQREPFGRQS*M-C(Sx)-EKR-Z (SEQ ID NO: 1921), X-FQREQFGRQS*M-C(Sx)-EKR-Z (SEQ ID NO: 1922), X-FQRERFGRQS*M-C(Sx)-EKR-Z (SEQ ID NO: 1923), X-FQREVFGRQS*M-C(Sx)-EKR-Z (SEQ ID NO: 1924), X-FQREWFGRQS*M-C(Sx)-EKR-Z (SEQ ID NO: 1925), X-FQRFGFGRQS*M-C(Sx)-EKR-Z (SEQ ID NO: 1926), X-FQRGGFGRQS*M-C(Sx)-EKR-Z (SEQ ID NO: 1927), X-FQRHGFGRQS*M-C(Sx)-EKR-Z (SEQ ID NO: 1928), X-FQRIGFGRQS*M-C(Sx)-EKR-Z (SEQ ID NO: 1929), X-FQRKGFGRQS*M-C(Sx)-EKR-Z (SEQ ID NO: 1930), X-FQRLGFGRQS*M-C(Sx)-EKR-Z (SEQ ID NO: 1931), X-FQRNGFGRQS*M-C(Sx)-EKR-Z (SEQ ID NO: 1932), X-FQRPGFGRQS*M-C(Sx)-EKR-Z (SEQ ID NO: 1933), X-FQRQGFGRQS*M-C(Sx)-EKR-Z (SEQ ID NO: 1934), X-FQRRGFGRQS*M-C(Sx)-EKR-Z (SEQ ID NO: 1935), X-FQRVGFGRQS*M-C(Sx)-EKR-Z (SEQ ID NO: 1936), X-FQRWGFGRQS*M-C(Sx)-EKR-Z (SEQ ID NO: 1937), X-FQVEGFGRQS*M-C(Sx)-EKR-Z (SEQ ID NO: 1938), X-FQWEGFGRQS*M-C(Sx)-EKR-Z (SEQ ID NO: 1939), X-FRR-C(Sx)-AS*FRRR-Z (SEQ ID NO: 1940), X-FRREGFGRQS*M-C(Sx)-EKR-Z (SEQ ID NO: 1941), X-FVREGFGRQS*M-C(Sx)-EKR-Z (SEQ ID NO: 1942), and X-FWREGFGRQS*M-C(Sx)-EKR-Z (SEQ ID NO: 1943); wherein X— is H or acetyl; and Z is OH or NH2.

In some embodiments, the metal-binding compound of the present invention is selected from the group consisting of: X-GAKTTKKRPQRATS*N-C(Sx)-FS-Z (SEQ ID NO: 1944), X-GAKTTKKRPQRATSN-C(Sx)-FS*-Z (SEQ ID NO: 1945), X-GANPLMA-C(Sx)-GT*LTRRHQNGRF-Z (SEQ ID NO: 1946), X-G-C(Sx)-DS*LRRKAK-Z (SEQ ID NO: 1947), X-G-C(Sx)-LS*PGPF-Z (SEQ ID NO: 1948), X-GDGS*D-C(Sx)-PY[pY]NSIPSK-Z (SEQ ID NO: 1949), X-GDGS*D-C(Sx)-PYYNSIPSK-Z (SEQ ID NO: 1950), X-GEKS*F-C(Sx)-RSVVGTPAY-Z (SEQ ID NO: 1951), X-GEKSF-C(Sx)-RS*VVGTPAY-Z (SEQ ID NO: 1165), X-GEKSFRRS*V-C(Sx)-GTPAY-Z (SEQ ID NO: 1164), X-GEKSFRRSV-C(Sx)-GT*PAY-Z (SEQ ID NO: 1952), X-GETSLMRT*L-C(Sx)-GTPTY-Z (SEQ ID NO: 1953), X-GETSLMRTL-C(Sx)-GT*PTY-Z (SEQ ID NO: 1954), X-GGPEPGPYA-C(Sx)-PS*VNT-Z (SEQ ID NO: 1955), X-GGSSGT*S-C(Sx)-EKGPE-Z (SEQ ID NO: 1187), X-GHVEEPAS*P-C(Sx)-AAYQK-Z (SEQ ID NO: 1070), X-GKKALRRQES*V-C(Sx)-ALGW-Z (SEQ ID NO: 1020), X-GKKAT-C(Sx)-AS*QEY-Z (SEQ ID NO: 1956), X-GKKATQAS*Q-C(Sx)-Y-Z (SEQ ID NO: 1127), X-GKRPQRAT*S-C(Sx)-VFAMF-Z (SEQ ID NO: 1957), X-GLLR-C(Sx)-SS*RRIRR-Z (SEQ ID NO: 1958), X-GLSGRKSS*T-C(Sx)-SPTSP-Z (SEQ ID NO: 1095), X-GNLLP[Nle]S*P-C(Sx)-EFD-Z (SEQ ID NO: 1959), X-GQL-C(Sx)-DS*[Nle]ANSFVGTR-Z (SEQ ID NO: 1960), X-GQLIDS*[Nle]-C(Sx)-NSFVGTR-Z (SEQ ID NO: 1961), X-GQLIDS[Nle]ANS*F-C(Sx)-GTR-Z (SEQ ID NO: 1962), X-GQLIDS[Nle]ANSF-C(Sx)-GT*R-Z (SEQ ID NO: 1963), X-GQLIDS[Nle]-C(Sx)-NS*FVGTR-Z (SEQ ID NO: 1964), X-GQREGFGRQS*M-C(Sx)-EKR-Z (SEQ ID NO: 1965), X-GRDK-C(Sx)-KT*LRQIRQG-Z (SEQ ID NO: 1966), X-GRDKYKT*L-C(Sx)-QIRQG-Z (SEQ ID NO: 1967), X-GREKR-C(Sx)-NS*QSYIGRP-Z (SEQ ID NO: 1159), X-GREKRSNS*Q-C(Sx)-YIGRP-Z (SEQ ID NO: 1158), X-GRPR-C(Sx)-SS*FAEG-Z (SEQ ID NO: 1968), X-GRR-C(Sx)-AS*FRRR-Z (SEQ ID NO: 1969), X-GSASASGS*P-C(Sx)-DPGF-Z (SEQ ID NO: 1970), X-GSNQEEAYV-C(Sx)-[Nle]S*SFY-Z (SEQ ID NO: 1971), X-GVRRR-C(Sx)-LS*NVSLTGV-Z (SEQ ID NO: 1155), X-GVRRRRLS*N-C(Sx)-SLTGV-Z (SEQ ID NO: 1154), X-GYAKDV-C(Sx)-QGS*LCTSFVGTLQYLAKKK-Z (SEQ ID NO: 1009), X-GYAKDVDQGS*L-C(Sx)-TSFVGTLQYLAKKK-Z (SEQ ID NO: 1010), X-GYAKDVDQGSL-C(Sx)-TS*FVGTLQYLAKKK-Z (SEQ ID NO: 1011), X-GYAKDVDQGSLCTS*F-C(Sx)-GTLQYLA-Z (SEQ ID NO: 1012), X-GYAKDVDQGSLCTSF-C(Sx)-GT*LQYLA-Z (SEQ ID NO: 1972), X-HAKTTKKRPQRATS*N-C(Sx)-FS-Z (SEQ ID NO: 1973), X-HAKTTKKRPQRATSN-C(Sx)-FS*-Z (SEQ ID NO: 1974), X-HANPLMA-C(Sx)-GT*LTRRHQNGRF-Z (SEQ ID NO: 1975), X-H-C(Sx)-DS*LRRKAK-Z (SEQ ID NO: 1976), X-H-C(Sx)-LS*PGPF-Z (SEQ ID NO: 1977), X-HKRPQRAT*S-C(Sx)-VFAMF-Z (SEQ ID NO: 1978), X-HLLR-C(Sx)-SS*RRIRR-Z (SEQ ID NO: 1979), X-HLPWWR-C(Sx)-YT*WVVERDVNTKQR-Z (SEQ ID NO: 1980), X-HQREGFGRQS*M-C(Sx)-EKR-Z (SEQ ID NO: 1981), X-HRR-C(Sx)-AS*FRRR-Z (SEQ ID NO: 1982), X-I[Nle]LR-C(Sx)-SS*RRIRR-Z (SEQ ID NO: 1983), X-IADLG-C(Sx)-AS*FK[Nle]WSKL-Z (SEQ ID NO: 1984), X-IADLG-C(Sx)-AS*FKMWSKL-Z (SEQ ID NO: 1985), X-IADLGLAS*F-C(Sx)-[Nle]WSKL-Z (SEQ ID NO: 1986), X-IAKTTKKRPQRATS*N-C(Sx)-FS-Z (SEQ ID NO: 1987), X-IAKTTKKRPQRATSN-C(Sx)-FS*-Z (SEQ ID NO: 1988), X-IALR-C(Sx)-SS*RRIRR-Z (SEQ ID NO: 1989), X-IANPLMA-C(Sx)-GT*LTRRHQNGRF-Z (SEQ ID NO: 1990), X-I-C(Sx)-DS*LRRKAK-Z (SEQ ID NO: 1991), X-I-C(Sx)-LS*PGPF-Z (SEQ ID NO: 1992), X-I-C(Sx)-TS*PITTYFFFKKK-Z (SEQ ID NO: 1193), X-I-C(Sx)-TT*PITITYFFFKKK-Z (SEQ ID NO: 1993), X-IDLR-C(Sx)-SS*RRIRR-Z (SEQ ID NO: 1994), X-IDQGD-C(Sx)-MT*PQFTPYY-Z (SEQ ID NO: 1025), X-IDQGDLMT*P-C(Sx)-FTPYY-Z (SEQ ID NO: 1024); wherein X— is H or acetyl; and Z is OH or NH2.

In some embodiments, the metal-binding compound of the present invention is selected from the group consisting of: X-IELR-C(Sx)-SS*RRIRR-Z (SEQ ID NO: 1995), X-IFLR-C(Sx)-SS*RRIRR-Z (SEQ ID NO: 1996), X-IGLR-C(Sx)-SS*RRIRR-Z (SEQ ID NO: 1997), X-IHLR-C(Sx)-SS*RRIRR-Z (SEQ ID NO: 1998), X-IIHRD-C(Sx)-KS*NNIFLHE-Z (SEQ ID NO: 1999), X-IIHRDLKS*N-C(Sx)-IFLHE-Z (SEQ ID NO: 2000), X-IILR-C(Sx)-SS*RRIRR-Z (SEQ ID NO: 2001), X-IKLR-C(Sx)-SS*RRIRR-Z (SEQ ID NO: 2002), X-IKRPQRAT*S-C(Sx)-VFAMF-Z (SEQ ID NO: 2003), X-IL[Nle]R-C(Sx)-SS*RRIRR-Z (SEQ ID NO: 2004), X-ILAR-C(Sx)-SS*RRIRR-Z (SEQ ID NO: 2005), X-ILDR-C(Sx)-SS*RRIRR-Z (SEQ ID NO: 2006), X-ILER-C(Sx)-SS*RRIRR-Z (SEQ ID NO: 2007), X-ILFR-C(Sx)-SS*RRIRR-Z (SEQ ID NO: 2008), X-ILGR-C(Sx)-SS*RRIRR-Z (SEQ ID NO: 2009), X-ILHR-C(Sx)-SS*RRIRR-Z (SEQ ID NO: 2010), X-ILIR-C(Sx)-SS*RRIRR-Z (SEQ ID NO: 2011), X-ILKR-C(Sx)-SS*RRIRR-Z (SEQ ID NO: 2012), X-ILL[Nle]-C(Sx)-SS*RRIRR-Z (SEQ ID NO: 2013), X-ILL[Nle]ESS*R-C(Sx)-RIRSGNKLGRIGRN-Z (SEQ ID NO: 2014), X-ILL[pS]ESS*R-C(Sx)-RIRSGNKLGR-Z (SEQ ID NO: 2015), X-ILLA-C(Sx)-SS*RRIRR-Z (SEQ ID NO: 2016), X-ILLAESS*R-C(Sx)-RIRSGNKLGRIGRN-Z (SEQ ID NO: 2017), X-ILLD-C(Sx)-SS*RRIRR-Z (SEQ ID NO: 2018), X-ILLDESS*R-C(Sx)-RIRSGNKLGRIGRN-Z (SEQ ID NO: 2019), X-ILLE-C(Sx)-SS*RRIRR-Z (SEQ ID NO: 2020), X-ILLEESS*R-C(Sx)-RIRSGNKLGRIGRN-Z (SEQ ID NO: 2021), X-ILLF-C(Sx)-SS*RRIRR-Z (SEQ ID NO: 2022), X-ILLFESS*R-C(Sx)-RIRSGNKLGRIGRN-Z (SEQ ID NO: 2023), X-ILLG-C(Sx)-SS*RRIRR-Z (SEQ ID NO: 2024), X-ILLGESS*R-C(Sx)-RIRSGNKLGRIGRN-Z (SEQ ID NO: 2025), X-ILLH-C(Sx)-SS*RRIRR-Z (SEQ ID NO: 2026), X-ILLHESS*R-C(Sx)-RIRSGNKLGRIGRN-Z (SEQ ID NO: 2027), X-ILLI-C(Sx)-SS*RRIRR-Z (SEQ ID NO: 2028), X-ILLIESS*R-C(Sx)-RIRSGNKLGRIGRN-Z (SEQ ID NO: 2029), X-ILLK-C(Sx)-SS*RRIRR-Z (SEQ ID NO: 2030), X-ILLKESS*R-C(Sx)-RIRSGNKLGRIGRN-Z (SEQ ID NO: 2031), X-ILLL-C(Sx)-SS*RRIRR-Z (SEQ ID NO: 2032), X-ILLLESS*R-C(Sx)-RIRSGNKLGRIGRN-Z (SEQ ID NO: 2033), X-ILLN-C(Sx)-SS*RRIRR-Z (SEQ ID NO: 2034), X-ILLNESS*R-C(Sx)-RIRSGNKLGRIGRN-Z (SEQ ID NO: 2035), X-ILLP-C(Sx)-SS*RRIRR-Z (SEQ ID NO: 2036), X-ILLPESS*R-C(Sx)-RIRSGNKLGRIGRN-Z (SEQ ID NO: 2037), X-ILLQ-C(Sx)-SS*RRIRR-Z (SEQ ID NO: 2038), X-ILLQESS*R-C(Sx)-RIRSGNKLGRIGRN-Z (SEQ ID NO: 2039), X-ILLR-C(Sx)-[Nle]S*RRIRR-Z (SEQ ID NO: 2040), X-ILLR-C(Sx)-AS*RRIRR-Z (SEQ ID NO: 2041), X-ILLR-C(Sx)-DS*RRIRR-Z (SEQ ID NO: 2042), X-ILLR-C(Sx)-ES*RRIRR-Z (SEQ ID NO: 2043), X-ILLR-C(Sx)-FS*RRIRR-Z (SEQ ID NO: 2044), X-ILLR-C(Sx)-GS*RRIRR-Z (SEQ ID NO: 2045), X-ILLR-C(Sx)-HS*RRIRR-Z (SEQ ID NO: 2046), X-ILLR-C(Sx)-IS*RRIRR-Z (SEQ ID NO: 2047), X-ILLR-C(Sx)-KS*RRIRR-Z (SEQ ID NO: 2048), X-ILLR-C(Sx)-LS**RRIRR-Z (SEQ ID NO: 2049), X-ILLR-C(Sx)-NS*RRIRR-Z (SEQ ID NO: 2050), X-ILLR-C(Sx)-PS*RRIRR-Z (SEQ ID NO: 2051), X-ILLR-C(Sx)-QS*RRIRR-Z (SEQ ID NO: 2052), X-ILLR-C(Sx)-RS*RRIRR-Z (SEQ ID NO: 2053), X-ILLR-C(Sx)-SS*[Nle]RIRR-Z (SEQ ID NO: 2054), X-ILLR-C(Sx)-SS*ARIRR-Z (SEQ ID NO: 2055), X-ILLR-C(Sx)-SS*DRIRR-Z (SEQ ID NO: 2056), X-ILLR-C(Sx)-SS*ERIRR-Z (SEQ ID NO: 2057), X-ILLR-C(Sx)-SS*FRIRR-Z (SEQ ID NO: 2058), X-ILLR-C(Sx)-SS*GRIRR-Z (SEQ ID NO: 2059), X-ILLR-C(Sx)-SS*HRIRR-Z (SEQ ID NO: 2060), X-ILLR-C(Sx)-SS*IRIRR-Z (SEQ ID NO: 2061), X-ILLR-C(Sx)-SS*KRIRR-Z (SEQ ID NO: 2062), X-ILLR-C(Sx)-SS*LRIRR-Z (SEQ ID NO: 2063), X-ILLR-C(Sx)-SS*NRIRR-Z (SEQ ID NO: 2064), X-ILLR-C(Sx)-SS*PRIRR-Z (SEQ ID NO: 2065), X-ILLR-C(Sx)-SS*QRIRR-Z (SEQ ID NO: 2066), X-ILLR-C(Sx)-SS*R[Nle]IRR-Z (SEQ ID NO: 2067), and X-ILLR-C(Sx)-SS*RAIRR-Z (SEQ ID NO: 2068), X-ILLR-C(Sx)-SS*RDIRR-Z (SEQ ID NO: 2069); wherein X is H or acetyl; and Z is OH or NH2.

In some embodiments, the metal-binding compound of the present invention is selected from the group consisting of: X-ILLR-C(Sx)-SS*REIRR-Z (SEQ ID NO: 2070), X-ILLR-C(Sx)-SS*RFIRR-Z (SEQ ID NO: 2071), X-ILLR-C(Sx)-SS*RGIRR-Z (SEQ ID NO: 2072), X-ILLR-C(Sx)-SS*RHIRR-Z (SEQ ID NO: 2073), X-ILLR-C(Sx)-SS*RIIRR-Z (SEQ ID NO: 2074), X-ILLR-C(Sx)-SS*RKIRR-Z (SEQ ID NO: 2075), X-ILLR-C(Sx)-SS*RLIRR-Z (SEQ ID NO: 2076), X-ILLR-C(Sx)-SS*RNIRR-Z (SEQ ID NO: 2077), X-ILLR-C(Sx)-SS*RPIRR-Z (SEQ ID NO: 2078), X-ILLR-C(Sx)-SS*RQIRR-Z (SEQ ID NO: 2079), X-ILLR-C(Sx)-SS*RR[Nle]RR-Z (SEQ ID NO: 2080), X-ILLR-C(Sx)-SS*RRARR-Z (SEQ ID NO: 2081), X-ILLR-C(Sx)-SS*RRDRR-Z (SEQ ID NO: 2082), X-ILLR-C(Sx)-SS*RRERR-Z (SEQ ID NO: 2083), X-ILLR-C(Sx)-SS*RRFRR-Z (SEQ ID NO: 2084), X-ILLR-C(Sx)-SS*RRGRR-Z (SEQ ID NO: 2085), X-ILLR-C(Sx)-SS*RRHRR-Z (SEQ ID NO: 2086), X-ILLR-C(Sx)-SS*RRI[Nle]R-Z (SEQ ID NO: 2087), X-ILLR-C(Sx)-SS*RRIAR-Z (SEQ ID NO: 2088), X-ILLR-C(Sx)-SS*RRIDR-Z (SEQ ID NO: 2089), X-ILLR-C(Sx)-SS*RRIER-Z (SEQ ID NO: 2090), X-ILLR-C(Sx)-SS*RRIFR-Z (SEQ ID NO: 2091), X-ILLR-C(Sx)-SS*RRIGR-Z (SEQ ID NO: 2092), X-ILLR-C(Sx)-SS*RRIHR-Z (SEQ ID NO: 2093), X-ILLR-C(Sx)-SS*RRIIR-Z (SEQ ID NO: 2094), X-ILLR-C(Sx)-SS*RRIKR-Z (SEQ ID NO: 2095), X-ILLR-C(Sx)-SS*RRILR-Z (SEQ ID NO: 2096), X-ILLR-C(Sx)-SS*RRINR-Z (SEQ ID NO: 2097), X-ILLR-C(Sx)-SS*RRIPR-Z (SEQ ID NO: 2098), X-ILLR-C(Sx)-SS*RRIQR-Z (SEQ ID NO: 2099), X-ILLR-C(Sx)-SS*RRIR[Nle]-Z (SEQ ID NO: 2100), X-ILLR-C(Sx)-SS*RRIRA-Z (SEQ ID NO: 2101), X-ILLR-C(Sx)-SS*RRIRD-Z (SEQ ID NO: 2102), X-ILLR-C(Sx)-SS*RRIRE-Z (SEQ ID NO: 2103), X-ILLR-C(Sx)-SS*RRIRF-Z (SEQ ID NO: 2104), X-ILLR-C(Sx)-SS*RRIRG-Z (SEQ ID NO: 2105), X-ILLR-C(Sx)-SS*RRIRH-Z (SEQ ID NO: 2106), X-ILLR-C(Sx)-SS*RRIRI-Z (SEQ ID NO: 2107), X-ILLR-C(Sx)-SS*RRIRK-Z (SEQ ID NO: 2108), X-ILLR-C(Sx)-SS*RRIRL-Z (SEQ ID NO: 2109), X-ILLR-C(Sx)-SS*RRIRN-Z (SEQ ID NO: 2110), X-ILLR-C(Sx)-SS*RRIRP-Z (SEQ ID NO: 2111), X-ILLR-C(Sx)-SS*RRIRQ-Z (SEQ ID NO: 2112), X-ILLR-C(Sx)-SS*RRIRV-Z (SEQ ID NO: 2113), X-ILLR-C(Sx)-SS*RRIRW-Z (SEQ ID NO: 2114), X-ILLR-C(Sx)-SS*RRIVR-Z (SEQ ID NO: 2115), X-ILLR-C(Sx)-SS*RRIWR-Z (SEQ ID NO: 2116), X-ILLR-C(Sx)-SS*RRKRR-Z (SEQ ID NO: 2117), X-ILLR-C(Sx)-SS*RRLRR-Z (SEQ ID NO: 2118), X-ILLR-C(Sx)-SS*RRNRR-Z (SEQ ID NO: 2119), X-ILLR-C(Sx)-SS*RRPRR-Z (SEQ ID NO: 2120), X-ILLR-C(Sx)-SS*RRQRR-Z (SEQ ID NO: 2121), X-ILLR-C(Sx)-SS*RRRRR-Z (SEQ ID NO: 2122), X-ILLR-C(Sx)-SS*RRVRR-Z (SEQ ID NO: 2123), X-ILLR-C(Sx)-SS*RRWRR-Z (SEQ ID NO: 2124), X-ILLR-C(Sx)-SS*RVIRR-Z (SEQ ID NO: 2125), X-ILLR-C(Sx)-SS*RWIRR-Z (SEQ ID NO: 2126), X-ILLR-C(Sx)-SS*VRIRR-Z (SEQ ID NO: 2127), X-ILLR-C(Sx)-SS*WRIRR-Z (SEQ ID NO: 2128), X-ILLR-C(Sx)-VS*RRIRR-Z (SEQ ID NO: 2129), X-ILLR-C(Sx)-WS*RRIRR-Z (SEQ ID NO: 2130), X-ILLRESS*R-C(Sx)-RIRSGNKLGRIGRN-Z (SEQ ID NO: 2131), X-ILLS [Nle]SS*R-C(Sx)-RIRSGNKLGRIGRN-Z (SEQ ID NO: 2132), X-ILLSASS*R-C(Sx)-RIRSGNKLGRIGRN-Z (SEQ ID NO: 2133), X-ILLS-C(Sx)-LS*RRR-Z (SEQ ID NO: 2134), X-ILLSDSS*R-C(Sx)-RIRSGNKLGRIGRN-Z (SEQ ID NO: 2135), X-ILLSE[Nle]S*R-C(Sx)-RIRSGNKLGRIGRN-Z (SEQ ID NO: 2136), X-ILLSEAS*R-C(Sx)-RIRSGNKLGRIGRN-Z (SEQ ID NO: 2137), X-ILLSEDS*R-C(Sx)-RIRSGNKLGRIGRN-Z (SEQ ID NO: 2138), X-ILLSEES*R-C(Sx)-RIRSGNKLGRIGRN-Z (SEQ ID NO: 2139), X-ILLSEFS*R-C(Sx)-RIRSGNKLGRIGRN-Z (SEQ ID NO: 2140), X-ILLSEGS*R-C(Sx)-RIRSGNKLGRIGRN-Z (SEQ ID NO: 2141), X-ILLSEHS*R-C(Sx)-RIRSGNKLGRIGRN-Z (SEQ ID NO: 2142), X-ILLSEIS*R-C(Sx)-RIRSGNKLGRIGRN-Z (SEQ ID NO: 2143), X-ILLSEKS*R-C(Sx)-RIRSGNKLGRIGRN-Z (SEQ ID NO: 2144), X-ILLSELS*R-C(Sx)-RIRSGNKLGRIGRN-Z (SEQ ID NO: 2145), X-ILLSENS*R-C(Sx)-RIRSGNKLGRIGRN-Z (SEQ ID NO: 2146), X-ILLSEPS*R-C(Sx)-RIRSGNKLGRIGRN-Z (SEQ ID NO: 2147), X-ILLSEQS*R-C(Sx)-RIRSGNKLGRIGRN-Z (SEQ ID NO: 2148), X-ILLSERS*R-C(Sx)-RIRSGNKLGRIGRN-Z (SEQ ID NO: 2149); wherein X— is H or acetyl; and Z is OH or NH2.

In some embodiments, the metal-binding compound of the present invention is selected from the group consisting of: X-ILLSESS*[Nle]-C(Sx)-RIRSGNKLGRIGRN-Z (SEQ ID NO: 2150), X-ILLSESS*A-C(Sx)-RIRSGNKLGRIGRN-Z (SEQ ID NO: 2151), X-ILLSESS*D-C(Sx)-RIRSGNKLGRIGRN-Z (SEQ ID NO: 2152), X-ILLSESS*E-C(Sx)-RIRSGNKLGRIGRN-Z (SEQ ID NO: 2153), X-ILLSESS*F-C(Sx)-RIRSGNKLGRIGRN-Z (SEQ ID NO: 2154), X-ILLSESS*G-C(Sx)-RIRSGNKLGRIGRN-Z (SEQ ID NO: 2155), X-ILLSESS*H-C(Sx)-RIRSGNKLGRIGRN-Z (SEQ ID NO: 2156), X-ILLSESS*I-C(Sx)-RIRSGNKLGRIGRN-Z (SEQ ID NO: 2157), X-ILLSESS*K-C(Sx)-RIRSGNKLGRIGRN-Z (SEQ ID NO: 2158), X-ILLSESS*L-C(Sx)-RIRSGNKLGRIGRN-Z (SEQ ID NO: 2159), X-ILLSESS*N-C(Sx)-RIRSGNKLGRIGRN-Z (SEQ ID NO: 2160), X-ILLSESS*P-C(Sx)-RIRSGNKLGRIGRN-Z (SEQ ID NO: 2161), X-ILLSESS*Q-C(Sx)-RIRSGNKLGRIGRN-Z (SEQ ID NO: 2162), X-ILLSESS*R-C(Sx)-[Nle]IRSGNKLGRIGRN-Z (SEQ ID NO: 2163), X-ILLSESS*R-C(Sx)-AIRSGNKLGRIGRN-Z (SEQ ID NO: 2164), X-ILLSESS*R-C(Sx)-DIRSGNKLGRIGRN-Z (SEQ ID NO: 2165), X-ILLSESS*R-C(Sx)-EIRSGNKLGRIGRN-Z (SEQ ID NO: 2166), X-ILLSESS*R-C(Sx)-FIRSGNKLGRIGRN-Z (SEQ ID NO: 2167), X-ILLSESS*R-C(Sx)-GIRSGNKLGRIGRN-Z (SEQ ID NO: 2168), X-ILLSESS*R-C(Sx)-HIRSGNKLGRIGRN-Z (SEQ ID NO: 2169), X-ILLSESS*R-C(Sx)-IIRSGNKLGRIGRN-Z (SEQ ID NO: 2170), X-ILLSESS*R-C(Sx)-KIRSGNKLGRIGRN-Z (SEQ ID NO: 2171), X-ILLSESS*R-C(Sx)-LIRSGNKLGRIGRN-Z (SEQ ID NO: 2172), X-ILLSESS*R-C(Sx)-NIRSGNKLGRIGRN-Z (SEQ ID NO: 2173), X-ILLSESS*R-C(Sx)-PIRSGNKLGRIGRN-Z (SEQ ID NO: 2174), X-ILLSESS*R-C(Sx)-QIRSGNKLGRIGRN-Z (SEQ ID NO: 2175), X-ILLSESS*R-C(Sx)-R[Nle]RSGNKLGRIGRN-Z (SEQ ID NO: 2176), X-ILLSESS*R-C(Sx)-RARSGNKLGRIGRN-Z (SEQ ID NO: 2177), X-ILLSESS*R-C(Sx)-RDRSGNKLGRIGRN-Z (SEQ ID NO: 2178), X-ILLSESS*R-C(Sx)-RERSGNKLGRIGRN-Z (SEQ ID NO: 2179), X-ILLSESS*R-C(Sx)-RFRSGNKLGRIGRN-Z (SEQ ID NO: 2180), X-ILLSESS*R-C(Sx)-RGRSGNKLGRIGRN-Z (SEQ ID NO: 2181), X-ILLSESS*R-C(Sx)-RHRSGNKLGRIGRN-Z (SEQ ID NO: 2182), X-ILLSESS*R-C(Sx)-RI[Nle]SGNKLGRIGRN-Z (SEQ ID NO: 2183), X-ILLSESS*R-C(Sx)-RIASGNKLGRIGRN-Z (SEQ ID NO: 2184), X-ILLSESS*R-C(Sx)-RIDSGNKLGRIGRN-Z (SEQ ID NO: 2185), X-ILLSESS*R-C(Sx)-RIESGNKLGRIGRN-Z (SEQ ID NO: 2186), X-ILLSESS*R-C(Sx)-RIFSGNKLGRIGRN-Z (SEQ ID NO: 2187), X-ILLSESS*R-C(Sx)-RIGSGNKLGRIGRN-Z (SEQ ID NO: 2188), X-ILLSESS*R-C(Sx)-RIHSGNKLGRIGRN-Z (SEQ ID NO: 2189), X-ILLSESS*R-C(Sx)-RIISGNKLGRIGRN-Z (SEQ ID NO: 2190), X-ILLSESS*R-C(Sx)-RIKSGNKLGRIGRN-Z (SEQ ID NO: 2191), X-ILLSESS*R-C(Sx)-RILSGNKLGRIGRN-Z (SEQ ID NO: 2192), X-ILLSESS*R-C(Sx)-RINSGNKLGRIGRN-Z (SEQ ID NO: 2193), X-ILLSESS*R-C(Sx)-RIPSGNKLGRIGRN-Z (SEQ ID NO: 2194), X-ILLSESS*R-C(Sx)-RIQSGNKLGRIGRN-Z (SEQ ID NO: 2195), X-ILLSESS*R-C(Sx)-RIR[Nle]GNKLGRIGRN-Z (SEQ ID NO: 2196), X-ILLSESS*R-C(Sx)-RIRAGNKLGRIGRN-Z (SEQ ID NO: 2197), X-ILLSESS*R-C(Sx)-RIRDGNKLGRIGRN-Z (SEQ ID NO: 2198), X-ILLSESS*R-C(Sx)-RIREGNKLGRIGRN-Z (SEQ ID NO: 2199), X-ILLSESS*R-C(Sx)-RIRFGNKLGRIGRN-Z (SEQ ID NO: 2200), X-ILLSESS*R-C(Sx)-RIRGGNKLGRIGRN-Z (SEQ ID NO: 2201), X-ILLSESS*R-C(Sx)-RIRHGNKLGRIGRN-Z (SEQ ID NO: 2202), X-ILLSESS*R-C(Sx)-RIRIGNKLGRIGRN-Z (SEQ ID NO: 2203), X-ILLSESS*R-C(Sx)-RIRKGNKLGRIGRN-Z (SEQ ID NO: 2204), X-ILLSESS*R-C(Sx)-RIRLGNKLGRIGRN-Z (SEQ ID NO: 2205), X-ILLSESS*R-C(Sx)-RIRNGNKLGRIGRN-Z (SEQ ID NO: 2206), X-ILLSESS*R-C(Sx)-RIRPGNKLGRIGRN-Z (SEQ ID NO: 2207), X-ILLSESS*R-C(Sx)-RIRQGNKLGRIGRN-Z (SEQ ID NO: 2208), X-ILLSESS*R-C(Sx)-RIRRGNKLGRIGRN-Z (SEQ ID NO: 2209), and X-ILLSESS*R-C(Sx)-RIRS[Nle]NKLGRIGRN-Z (SEQ ID NO: 2210); wherein X is H or acetyl; and Z is OH or NH2.

In some embodiments, the metal-binding compound of the present invention is selected from the group consisting of: X-ILLSESS*R-C(Sx)-RIRSANKLGRIGRN-Z (SEQ ID NO: 2211), X-ILLSESS*R-C(Sx)-RIRSDNKLGRIGRN-Z (SEQ ID NO: 2212), X-ILLSESS*R-C(Sx)-RIRSENKLGRIGRN-Z (SEQ ID NO: 2213), X-ILLSESS*R-C(Sx)-RIRSFNKLGRIGRN-Z (SEQ ID NO: 2214), X-ILLSESS*R-C(Sx)-RIRSG[Nle]KLGRIGRN-Z (SEQ ID NO: 2215), X-ILLSESS*R-C(Sx)-RIRSGAKLGRIGRN-Z (SEQ ID NO: 2216), X-ILLSESS*R-C(Sx)-RIRSGDKLGRIGRN-Z (SEQ ID NO: 2217), X-ILLSESS*R-C(Sx)-RIRSGEKLGRIGRN-Z (SEQ ID NO: 2218), X-ILLSESS*R-C(Sx)-RIRSGFKLGRIGRN-Z (SEQ ID NO: 2219), X-ILLSESS*R-C(Sx)-RIRSGGKLGRIGRN-Z (SEQ ID NO: 2220), X-ILLSESS*R-C(Sx)-RIRSGHKLGRIGRN-Z (SEQ ID NO: 2221), X-ILLSESS*R-C(Sx)-RIRSGIKLGRIGRN-Z (SEQ ID NO: 2222), X-ILLSESS*R-C(Sx)-RIRSGKKLGRIGRN-Z (SEQ ID NO: 2223), X-ILLSESS*R-C(Sx)-RIRSGLKLGRIGRN-Z (SEQ ID NO: 2224), X-ILLSESS*R-C(Sx)-RIRSGN[Nle]LGRIGRN-Z (SEQ ID NO: 2225), X-ILLSESS*R-C(Sx)-RIRSGNALGRIGRN-Z (SEQ ID NO: 2226), X-ILLSESS*R-C(Sx)-RIRSGNDLGRIGRN-Z (SEQ ID NO: 2227), X-ILLSESS*R-C(Sx)-RIRSGNELGRIGRN-Z (SEQ ID NO: 2228), X-ILLSESS*R-C(Sx)-RIRSGNFLGRIGRN-Z (SEQ ID NO: 2229), X-ILLSESS*R-C(Sx)-RIRSGNGLGRIGRN-Z (SEQ ID NO: 2230), X-ILLSESS*R-C(Sx)-RIRSGNHLGRIGRN-Z (SEQ ID NO: 2231), X-ILLSESS*R-C(Sx)-RIRSGNILGRIGRN-Z (SEQ ID NO: 2232), X-ILLSESS*R-C(Sx)-RIRSGNK[Nle]GRIGRN-Z (SEQ ID NO: 2233), X-ILLSESS*R-C(Sx)-RIRSGNKAGRIGRN-Z (SEQ ID NO: 2234), X-ILLSESS*R-C(Sx)-RIRSGNKDGRIGRN-Z (SEQ ID NO: 2235), X-ILLSESS*R-C(Sx)-RIRSGNKEGRIGRN-Z (SEQ ID NO: 2236), X-ILLSESS*R-C(Sx)-RIRSGNKFGRIGRN-Z (SEQ ID NO: 2237), X-ILLSESS*R-C(Sx)-RIRSGNKGGRIGRN-Z (SEQ ID NO: 2238), X-ILLSESS*R-C(Sx)-RIRSGNKHGRIGRN-Z (SEQ ID NO: 2239), X-ILLSESS*R-C(Sx)-RIRSGNKIGRIGRN-Z (SEQ ID NO: 2240), X-ILLSESS*R-C(Sx)-RIRSGNKKGRIGRN-Z (SEQ ID NO: 2241), X-ILLSESS*R-C(Sx)-RIRSGNKL[Nle]RIGRN-Z (SEQ ID NO: 2242), X-ILLSESS*R-C(Sx)-RIRSGNKLARIGRN-Z (SEQ ID NO: 2243), X-ILLSESS*R-C(Sx)-RIRSGNKLDRIGRN-Z (SEQ ID NO: 2244), X-ILLSESS*R-C(Sx)-RIRSGNKLERIGRN-Z (SEQ ID NO: 2245), X-ILLSESS*R-C(Sx)-RIRSGNKLFRIGRN-Z (SEQ ID NO: 2246), X-ILLSESS*R-C(Sx)-RIRSGNKLG[Nle]IGRN-Z (SEQ ID NO: 2247), X-ILLSESS*R-C(Sx)-RIRSGNKLGAIGRN-Z (SEQ ID NO: 2248), X-ILLSESS*R-C(Sx)-RIRSGNKLGDIGRN-Z (SEQ ID NO: 2249), X-ILLSESS*R-C(Sx)-RIRSGNKLGEIGRN-Z (SEQ ID NO: 2250), X-ILLSESS*R-C(Sx)-RIRSGNKLGFIGRN-Z (SEQ ID NO: 2251), X-ILLSESS*R-C(Sx)-RIRSGNKLGGIGRN-Z (SEQ ID NO: 2252), X-ILLSESS*R-C(Sx)-RIRSGNKLGHIGRN-Z (SEQ ID NO: 2253), X-ILLSESS*R-C(Sx)-RIRSGNKLGIIGRN-Z (SEQ ID NO: 2254), X-ILLSESS*R-C(Sx)-RIRSGNKLGKIGRN-Z (SEQ ID NO: 2255), X-ILLSESS*R-C(Sx)-RIRSGNKLGLIGRN-Z (SEQ ID NO: 2256), X-ILLSESS*R-C(Sx)-RIRSGNKLGNIGRN-Z (SEQ ID NO: 2257), X-ILLSESS*R-C(Sx)-RIRSGNKLGPIGRN-Z (SEQ ID NO: 2258), X-ILLSESS*R-C(Sx)-RIRSGNKLGQIGRN-Z (SEQ ID NO: 2259), X-ILLSESS*R-C(Sx)-RIRSGNKLGR[Nle]GRN-Z (SEQ ID NO: 2260), X-ILLSESS*R-C(Sx)-RIRSGNKLGRAGRN-Z (SEQ ID NO: 2261), X-ILLSESS*R-C(Sx)-RIRSGNKLGRDGRN-Z (SEQ ID NO: 2262), X-ILLSESS*R-C(Sx)-RIRSGNKLGREGRN-Z (SEQ ID NO: 2263), X-ILLSESS*R-C(Sx)-RIRSGNKLGRFGRN-Z (SEQ ID NO: 2264), X-ILLSESS*R-C(Sx)-RIRSGNKLGRGGRN-Z (SEQ ID NO: 2265), X-ILLSESS*R-C(Sx)-RIRSGNKLGRHGRN-Z (SEQ ID NO: 2266), X-ILLSESS*R-C(Sx)-RIRSGNKLGRIGRN-Z (SEQ ID NO: 2267), X-ILLSESS*R-C(Sx)-RIRSGNKLGRKGRN-Z (SEQ ID NO: 2268), X-ILLSESS*R-C(Sx)-RIRSGNKLGRLGRN-Z (SEQ ID NO: 2269), X-ILLSESS*R-C(Sx)-RIRSGNKLGRNGRN-Z (SEQ ID NO: 2270), X-ILLSESS*R-C(Sx)-RIRSGNKLGRPGRN-Z (SEQ ID NO: 2271), and X-ILLSESS*R-C(Sx)-RIRSGNKLGRQGRN-Z (SEQ ID NO: 2272); wherein X is H or acetyl; and Z is OH or NH2.

In some embodiments, the metal-binding compound of the present invention is selected from the group consisting of: X-ILLSESS*R-C(Sx)-RIRSGNKLGRRGRN-Z (SEQ ID NO: 2273), X-ILLSESS*R-C(Sx)-RIRSGNKLGRVGRN-Z (SEQ ID NO: 2274), X-ILLSESS*R-C(Sx)-RIRSGNKLGRWGRN-Z (SEQ ID NO: 2275), X-ILLSESS*R-C(Sx)-RIRSGNKLGR-Z (SEQ ID NO: 2276), X-ILLSESS*R-C(Sx)-RIRSGNKLGVIGRN-Z (SEQ ID NO: 2277), X-ILLSESS*R-C(Sx)-RIRSGNKLGWIGRN-Z (SEQ ID NO: 2278), X-ILLSESS*R-C(Sx)-RIRSGNKLHRIGRN-Z (SEQ ID NO: 2279), X-ILLSESS*R-C(Sx)-RIRSGNKLIRIGRN-Z (SEQ ID NO: 2280), X-ILLSESS*R-C(Sx)-RIRSGNKLKRIGRN-Z (SEQ ID NO: 2281), X-ILLSESS*R-C(Sx)-RIRSGNKLLRIGRN-Z (SEQ ID NO: 2282), X-ILLSESS*R-C(Sx)-RIRSGNKLNRIGRN-Z (SEQ ID NO: 2283), X-ILLSESS*R-C(Sx)-RIRSGNKLPRIGRN-Z (SEQ ID NO: 2284), X-ILLSESS*R-C(Sx)-RIRSGNKLQRIGRN-Z (SEQ ID NO: 2285), X-ILLSESS*R-C(Sx)-RIRSGNKLRRIGRN-Z (SEQ ID NO: 2286), X-ILLSESS*R-C(Sx)-RIRSGNKLVRIGRN-Z (SEQ ID NO: 2287), X-ILLSESS*R-C(Sx)-RIRSGNKLWRIGRN-Z (SEQ ID NO: 2288), X-ILLSESS*R-C(Sx)-RIRSGNKNGRIGRN-Z (SEQ ID NO: 2289), X-ILLSESS*R-C(Sx)-RIRSGNKPGRIGRN-Z (SEQ ID NO: 2290), X-ILLSESS*R-C(Sx)-RIRSGNKQGRIGRN-Z (SEQ ID NO: 2291), X-ILLSESS*R-C(Sx)-RIRSGNKRGRIGRN-Z (SEQ ID NO: 2292), X-ILLSESS*R-C(Sx)-RIRSGNKVGRIGRN-Z (SEQ ID NO: 2293), X-ILLSESS*R-C(Sx)-RIRSGNKWGRIGRN-Z (SEQ ID NO: 2294), X-ILLSESS*R-C(Sx)-RIRSGNLLGRIGRN-Z (SEQ ID NO: 2295), X-ILLSESS*R-C(Sx)-RIRSGNNLGRIGRN-Z (SEQ ID NO: 2296), X-ILLSESS*R-C(Sx)-RIRSGNPLGRIGRN-Z (SEQ ID NO: 2297), X-ILLSESS*R-C(Sx)-RIRSGNQLGRIGRN-Z (SEQ ID NO: 2298), X-ILLSESS*R-C(Sx)-RIRSGNRLGRIGRN-Z (SEQ ID NO: 2299), X-ILLSESS*R-C(Sx)-RIRSGNVLGRIGRN-Z (SEQ ID NO: 2300), X-ILLSESS*R-C(Sx)-RIRSGNWLGRIGRN-Z (SEQ ID NO: 2301), X-ILLSESS*R-C(Sx)-RIRSGPKLGRIGRN-Z (SEQ ID NO: 2302), X-ILLSESS*R-C(Sx)-RIRSGQKLGRIGRN-Z (SEQ ID NO: 2303), X-ILLSESS*R-C(Sx)-RIRSGRKLGRIGRN-Z (SEQ ID NO: 2304), X-ILLSESS*R-C(Sx)-RIRSGVKLGRIGRN-Z (SEQ ID NO: 2305), X-ILLSESS*R-C(Sx)-RIRSGWKLGRIGRN-Z (SEQ ID NO: 2306), X-ILLSESS*R-C(Sx)-RIRSHNKLGRIGRN-Z (SEQ ID NO: 2307), X-ILLSESS*R-C(Sx)-RIRSINKLGRIGRN-Z (SEQ ID NO: 2308), X-ILLSESS*R-C(Sx)-RIRSKNKLGRIGRN-Z (SEQ ID NO: 2309), X-ILLSESS*R-C(Sx)-RIRSLNKLGRIGRN-Z (SEQ ID NO: 2310), X-ILLSESS*R-C(Sx)-RIRSNNKLGRIGRN-Z (SEQ ID NO: 2311), X-ILLSESS*R-C(Sx)-RIRSPNKLGRIGRN-Z (SEQ ID NO: 2312), X-ILLSESS*R-C(Sx)-RIRSQNKLGRIGRN-Z (SEQ ID NO: 2313), X-ILLSESS*R-C(Sx)-RIRSRNKLGRIGRN-Z (SEQ ID NO: 2314), X-ILLSESS*R-C(Sx)-RIRSVNKLGRIGRN-Z (SEQ ID NO: 2315), X-ILLSESS*R-C(Sx)-RIRSWNKLGRIGRN-Z (SEQ ID NO: 2316), X-ILLSESS*R-C(Sx)-RIRVGNKLGRIGRN-Z (SEQ ID NO: 2317), X-ILLSESS*R-C(Sx)-RIRWGNKLGRIGRN-Z (SEQ ID NO: 2318), X-ILLSESS*R-C(Sx)-RIVSGNKLGRIGRN-Z (SEQ ID NO: 2319), X-ILLSESS*R-C(Sx)-RIWSGNKLGRIGRN-Z (SEQ ID NO: 2320), X-ILLSESS*R-C(Sx)-RKRSGNKLGRIGRN-Z (SEQ ID NO: 2321), X-ILLSESS*R-C(Sx)-RLRSGNKLGRIGRN-Z (SEQ ID NO: 2322), and X-ILLSESS*R-C(Sx)-RNRSGNKLGRIGRN-Z (SEQ ID NO: 2323); wherein X is H or acetyl; and Z is OH or NH2.

In some embodiments, the metal-binding compound of the present invention is selected from the group consisting of: X-ILLSESS*R-C(Sx)-RPRSGNKLGRIGRN-Z (SEQ ID NO: 2324), X-ILLSESS*R-C(Sx)-RQRSGNKLGRIGRN-Z (SEQ ID NO: 2325), X-ILLSESS*R-C(Sx)-RRRSGNKLGRIGRN-Z (SEQ ID NO: 2326), X-ILLSESS*R-C(Sx)-RVRSGNKLGRIGRN-Z (SEQ ID NO: 2327), X-ILLSESS*R-C(Sx)-RWRSGNKLGRIGRN-Z (SEQ ID NO: 2328), X-ILLSESS*R-C(Sx)-VIRSGNKLGRIGRN-Z (SEQ ID NO: 2329), X-ILLSESS*R-C(Sx)-WIRSGNKLGRIGRN-Z (SEQ ID NO: 2330), X-ILLSESS*V-C(Sx)-RIRSGNKLGRIGRN-Z (SEQ ID NO: 2331), X-ILLSESS*W-C(Sx)-RIRSGNKLGRIGRN-Z (SEQ ID NO: 2332), X-ILLSEVS*R-C(Sx)-RIRSGNKLGRIGRN-Z (SEQ ID NO: 2333), X-ILLSEWS*R-C(Sx)-RIRSGNKLGRIGRN-Z (SEQ ID NO: 2334), X-ILLSFSS*R-C(Sx)-RIRSGNKLGRIGRN-Z (SEQ ID NO: 2335), X-ILLSGSS*R-C(Sx)-RIRSGNKLGRIGRN-Z (SEQ ID NO: 2336), X-ILLSHSS*R-C(Sx)-RIRSGNKLGRIGRN-Z (SEQ ID NO: 2337), X-ILLSISS*R-C(Sx)-RIRSGNKLGRIGRN-Z (SEQ ID NO: 2338), X-ILLSKSS*R-C(Sx)-RIRSGNKLGRIGRN-Z (SEQ ID NO: 2339), X-ILLSLSS*R-C(Sx)-RIRSGNKLGRIGRN-Z (SEQ ID NO: 2340), X-ILLSNSS*R-C(Sx)-RIRSGNKLGRIGRN-Z (SEQ ID NO: 2341), X-ILLSPSS*R-C(Sx)-RIRSGNKLGRIGRN-Z (SEQ ID NO: 2342), X-ILLSQSS*R-C(Sx)-RIRSGNKLGRIGRN-Z (SEQ ID NO: 2343), X-ILLSRSS*R-C(Sx)-RIRSGNKLGRIGRN-Z (SEQ ID NO: 2344), X-ILLSVSS*R-C(Sx)-RIRSGNKLGRIGRN-Z (SEQ ID NO: 2345), X-ILLSWSS*R-C(Sx)-RIRSGNKLGRIGRN-Z (SEQ ID NO: 2346), X-ILLV-C(Sx)-SS*RRIRR-Z (SEQ ID NO: 2347), X-ILLVESS*R-C(Sx)-RIRSGNKLGRIGRN-Z (SEQ ID NO: 2348), X-ILLW-C(Sx)-SS*RRIRR-Z (SEQ ID NO: 2349), X-ILLWESS*R-C(Sx)-RIRSGNKLGRIGRN-Z (SEQ ID NO: 2350), X-ILNR-C(Sx)-SS*RRIRR-Z (SEQ ID NO: 2351), X-ILPR-C(Sx)-SS*RRIRR-Z (SEQ ID NO: 2352), X-ILPWWR-C(Sx)-YT*WVVERDVNTKQR-Z (SEQ ID NO: 2353), X-ILQR-C(Sx)-SS*RRIRR-Z (SEQ ID NO: 2354), X-ILRR-C(Sx)-SS*RRIRR-Z (SEQ ID NO: 2355), X-ILVR-C(Sx)-SS*RRIRR-Z (SEQ ID NO: 2356), X-ILWR-C(Sx)-SS*RRIRR-Z (SEQ ID NO: 2357), X-INLR-C(Sx)-SS*RRIRR-Z (SEQ ID NO: 2358), X-IPLR-C(Sx)-SS*RRIRR-Z (SEQ ID NO: 2359); wherein X— is H or acetyl; and Z is OH or NH2.

In some embodiments, the metal-binding compound of the present invention is selected from the group consisting of: X-IPTT*P-C(Sx)-TTTYFFFKKK-Z (SEQ ID NO: 2360), X-IPTTP-C(Sx)-TT*TYFFFKKK-Z (SEQ ID NO: 2361), X-IQLR-C(Sx)-SS*RRIRR-Z (SEQ ID NO: 2362), X-IQREGFGRQS*M-C(Sx)-EKR-Z (SEQ ID NO: 2363), X-IRLR-C(Sx)-SS*RRIRR-Z (SEQ ID NO: 2364), X-IRR-C(Sx)-AS*FRRR-Z (SEQ ID NO: 2365), X-IS*G-C(Sx)-LSPI[Nle]TEQ-Z (SEQ ID NO: 2366), X-ISG-C(Sx)-LS*PI[Nle]TEQ-Z (SEQ ID NO: 2367), X-ISGRLS*P-C(Sx)-[Nle]TEQ-Z (SEQ ID NO: 2368), X-ISGRLSP-C(Sx)-[Nle]T*EQ-Z (SEQ ID NO: 2369), X-IVLR-C(Sx)-SS*RRIRR-Z (SEQ ID NO: 2370), X-IWLR-C(Sx)-SS*RRIRR-Z (SEQ ID NO: 2371), X-K[Nle]EVEELS*P-C(Sx)-ALGRF-Z (SEQ ID NO: 2372), X-K[Nle]KTTKKRPQRATS*N-C(Sx)-FS-Z (SEQ ID NO: 2373), X-K[Nle]KTTKKRPQRATSN-C(Sx)-FS*-Z (SEQ ID NO: 2374), X-K[Nle]RPQRAT*S-C(Sx)-VFAMF-Z (SEQ ID NO: 2375), X-KA[Nle]TTKKRPQRATS*N-C(Sx)-FS-Z (SEQ ID NO: 2376), X-KA[Nle]TTKKRPQRATSN-C(Sx)-FS*-Z (SEQ ID NO: 2377), X-KADTTKKRPQRATS*N-C(Sx)-FS-Z (SEQ ID NO: 2378), X-KADTTKKRPQRATSN-C(Sx)-FS*-Z (SEQ ID NO: 2379), X-KAETTKKRPQRATS*N-C(Sx)-FS-Z (SEQ ID NO: 2380), X-KAETTKKRPQRATSN-C(Sx)-FS*-Z (SEQ ID NO: 2381), X-KAFTTKKRPQRATS*N-C(Sx)-FS-Z (SEQ ID NO: 2382), X-KAFTTKKRPQRATSN-C(Sx)-FS*-Z (SEQ ID NO: 2383), X-KAGTTKKRPQRATS*N-C(Sx)-FS-Z (SEQ ID NO: 2384), X-KAGTTKKRPQRATSN-C(Sx)-FS*-Z (SEQ ID NO: 2385), X-KAHTTKKRPQRATS*N-C(Sx)-FS-Z (SEQ ID NO: 2386), X-KAHTTKKRPQRATSN-C(Sx)-FS*-Z (SEQ ID NO: 2387), X-KAITTKKRPQRATS*N-C(Sx)-FS-Z (SEQ ID NO: 2388), X-KAITTKKRPQRATSN-C(Sx)-FS*-Z (SEQ ID NO: 2389), X-KAK[Nle]TKKRPQRATS*N-C(Sx)-FS-Z (SEQ ID NO: 2390), X-KAK[Nle]TKKRPQRATSN-C(Sx)-FS*-Z (SEQ ID NO: 2391), X-KAKDTKKRPQRATS*N-C(Sx)-FS-Z (SEQ ID NO: 2392), X-KAKDTKKRPQRATSN-C(Sx)-FS*-Z (SEQ ID NO: 2393), X-KAKETKKRPQRATS*N-C(Sx)-FS-Z (SEQ ID NO: 2394), X-KAKETKKRPQRATSN-C(Sx)-FS*-Z (SEQ ID NO: 2395), X-KAKFTKKRPQRATS*N-C(Sx)-FS-Z (SEQ ID NO: 2396), X-KAKFTKKRPQRATSN-C(Sx)-FS*-Z (SEQ ID NO: 2397), X-KAKGTKKRPQRATS*N-C(Sx)-FS-Z (SEQ ID NO: 2398), X-KAKGTKKRPQRATSN-C(Sx)-FS*-Z (SEQ ID NO: 2399), X-KAKHTKKRPQRATS*N-C(Sx)-FS-Z (SEQ ID NO: 2400), X-KAKHTKKRPQRATSN-C(Sx)-FS*-Z (SEQ ID NO: 2401), X-KAKITKKRPQRATS*N-C(Sx)-FS-Z (SEQ ID NO: 2402), X-KAKITKKRPQRATSN-C(Sx)-FS*-Z (SEQ ID NO: 2403), X-KAKKKKKRPQRADS*D-C(Sx)-FA-Z (SEQ ID NO: 2404), X-KAKKKKKRPQRADS*D-C(Sx)-FS-Z (SEQ ID NO: 2405), X-KAKKKKKRPQRADS*N-C(Sx)-FA-Z (SEQ ID NO: 2406), X-KAKKKKKRPQRADS*N-C(Sx)-FS-Z (SEQ ID NO: 2407), X-KAKKKKKRPQRADSD-C(Sx)-FS*-Z (SEQ ID NO: 2408), X-KAKKKKKRPQRADSN-C(Sx)-FS*-Z (SEQ ID NO: 2409), X-KAKKKKKRPQRAES*D-C(Sx)-FA-Z (SEQ ID NO: 2410), X-KAKKKKKRPQRAES*D-C(Sx)-FS-Z (SEQ ID NO: 2411), X-KAKKKKKRPQRAES*N-C(Sx)-FA-Z (SEQ ID NO: 2412), X-KAKKKKKRPQRAES*N-C(Sx)-FS-Z (SEQ ID NO: 2413), X-KAKKKKKRPQRAESD-C(Sx)-FS*-Z (SEQ ID NO: 2414), X-KAKKKKKRPQRAESN-C(Sx)-FS*-Z (SEQ ID NO: 2415), X-KAKKKKKRPQRAHS*D-C(Sx)-FA-Z (SEQ ID NO: 2416), X-KAKKKKKRPQRAHS*D-C(Sx)-FS-Z (SEQ ID NO: 2417), X-KAKKKKKRPQRAHS*N-C(Sx)-FA-Z (SEQ ID NO: 2418), X-KAKKKKKRPQRAHS*N-C(Sx)-FS-Z (SEQ ID NO: 2419), and X-KAKKKKKRPQRAHSD-C(Sx)-FS*-Z (SEQ ID NO: 2420); wherein X is H or acetyl; and Z is OH or NH2.

In some embodiments, the metal-binding compound of the present invention is selected from the group consisting of: X-KAKKKKKRPQRAHSN-C(Sx)-FS*-Z (SEQ ID NO: 2421), X-KAKKKKKRPQRATS*D-C(Sx)-FA-Z (SEQ ID NO: 2422), X-KAKKKKKRPQRATS*D-C(Sx)-FS-Z (SEQ ID NO: 2423), X-KAKKKKKRPQRATS*N-C(Sx)-FA-Z (SEQ ID NO: 2424), X-KAKKKKKRPQRATS*N-C(Sx)-FS-Z (SEQ ID NO: 2425), X-KAKKKKKRPQRATSD-C(Sx)-FS*-Z (SEQ ID NO: 2426), X-KAKKKKKRPQRATSN-C(Sx)-FS*-Z (SEQ ID NO: 2427), X-KAKKRAGGANS*N-C(Sx)-FS[Nle]F-Z (SEQ ID NO: 2428), X-KAKKRAGGANS*N-C(Sx)-FSMF-Z (SEQ ID NO: 2429), X-KAKKRAGGANS*N-C(Sx)-FS-Z (SEQ ID NO: 2430), X-KAKKRAGGANSN-C(Sx)-FS*[Nle]F-Z (SEQ ID NO: 2431), X-KAKKRAGGANSN-C(Sx)-FS*MF-Z (SEQ ID NO: 2432), X-KAKKRAGGANSN-C(Sx)-FS*-Z (SEQ ID NO: 2433), X-KAKKRAGGANSNVFS*[Nle]-C(Sx)-EQ-Z (SEQ ID NO: 2434), X-KAKKRAGGANSNVFS*[Nle]-C(Sx)-Z (SEQ ID NO: 2435), X-KAKKRAGGANSNVFS*M-C(Sx)-EQ-Z (SEQ ID NO: 2436), X-KAKKRAGGANSNVFS*M-C(Sx)-Z (SEQ ID NO: 2437), X-KAKKRAGG-C(Sx)-NS*NVFS[Nle]F-Z (SEQ ID NO: 2438), X-KAKKRAGG-C(Sx)-NS*NVFSMF-Z (SEQ ID NO: 2439), X-KAKKRAGG-C(Sx)-NS*NVFS-Z (SEQ ID NO: 2440), X-KCAKKRKKRPQRADS*D-C(Sx)-FA-Z (SEQ ID NO: 2441), X-KCAKKRKKRPQRADS*D-C(Sx)-FS-Z (SEQ ID NO: 2442), X-KCAKKRKKRPQRADS*N-C(Sx)-FA-Z (SEQ ID NO: 2443), X-KAKKRKKRPQRADS*N-C(Sx)-FS-Z (SEQ ID NO: 2444), X-KCAKKRKKRPQRADSD-C(Sx)-FS*-Z (SEQ ID NO: 2445), X-KAKKRKKRPQRADSN-C(Sx)-FS*-Z (SEQ ID NO: 2446), X-KAKKRKKRPQRAES*D-C(Sx)-FA-Z (SEQ ID NO: 2447), X-KAKKRKKRPQRAES*D-C(Sx)-FS-Z (SEQ ID NO: 2448), X-KCAKKRKKRPQRAES*N-C(Sx)-FA-Z (SEQ ID NO: 2449), X-KAKKRKKRPQRAES*N-C(Sx)-FS-Z (SEQ ID NO: 2450), X-KCAKKRKKRPQRAESD-C(Sx)-FS*-Z (SEQ ID NO: 2451), X-KAKKRKKRPQRAESN-C(Sx)-FS*-Z (SEQ ID NO: 2452), X-KAKKRKKRPQRAHS*D-C(Sx)-FA-Z (SEQ ID NO: 2453), X-KAKKRKKRPQRAHS*D-C(Sx)-FS-Z (SEQ ID NO: 2454), X-KCAKKRKKRPQRAHS*N-C(Sx)-FA-Z (SEQ ID NO: 2455), X-KAKKRKKRPQRAHS*N-C(Sx)-FS-Z (SEQ ID NO: 2456), X-KCAKKRKKRPQRAHSD-C(Sx)-FS*-Z (SEQ ID NO: 2457), X-KAKKRKKRPQRAHSN-C(Sx)-FS*-Z (SEQ ID NO: 2458), X-KAKKRKKRPQRATS*D-C(Sx)-FA-Z (SEQ ID NO: 2459), X-KAKKRKKRPQRATS*D-C(Sx)-FS-Z (SEQ ID NO: 2460), X-KAKKRKKRPQRATS*N-C(Sx)-FA-Z (SEQ ID NO: 2461), X-KAKKRKKRPQRATS*N-C(Sx)-FS-Z (SEQ ID NO: 2462), X-KCAKKRKKRPQRATSD-C(Sx)-FS*-Z (SEQ ID NO: 2463), X-KAKKRKKRPQRATSN-C(Sx)-FS*-Z (SEQ ID NO: 2464), X-KKKTKKRPQRADS*D-(Sx)-FA-Z (SEQ ID NO: 2465), X-KAKKTKKRPQRADS*D-C(Sx)-FS-Z (SEQ ID NO: 2466), X-KAKKTKKRPQRADS*N-C(Sx)-FA-Z (SEQ ID NO: 2467), X-KAKKTKKRPQRADS*N-C(Sx)-FS-Z (SEQ ID NO: 2468), X-KAKKTKKRPQRADSD-C(Sx)-FS*-Z (SEQ ID NO: 2469), X-KAKKTKKRPQRADSN-C(Sx)-FS*-Z (SEQ ID NO: 2470), X-KAKKTKKRPQRAES*D-C(Sx)-FA-Z (SEQ ID NO: 2471), X-KAKKTKKRPQRAES*D-C(Sx)-FS-Z (SEQ ID NO: 2472), X-KAKKTKKRPQRAES*N-C(Sx)-FA-Z (SEQ ID NO: 2473), X-KAKKTKKRPQRAES*N-C(Sx)-FS-Z (SEQ ID NO: 2474), X-KAKKTKKRPQRAESD-C(Sx)-FS*-Z (SEQ ID NO: 2475), X-KAKKTKKRPQRAESN-C(Sx)-FS*-Z (SEQ ID NO: 2476), X-KKKTKKRPQRAHS*D-(Sx)-FA-Z (SEQ ID NO: 2477), X-KAKKTKKRPQRAHS*D-C(Sx)-FS-Z (SEQ ID NO: 2478), X-KAKKTKKRPQRAHS*N-C(Sx)-FA-Z (SEQ ID NO: 2479), X-KAKKTKKRPQRAHS*N-C(Sx)-FS-Z (SEQ ID NO: 2480), X-KAKKTKKRPQRAHSD-C(Sx)-FS*-Z (SEQ ID NO: 2481), X-KAKKTKKRPQRAHSN-C(Sx)-FS*-Z (SEQ ID NO: 2482), X-KAKKTKKRPQRATS*D-C(Sx)-FA-Z (SEQ ID NO: 2483), X-KAKKTKKRPQRATS*D-C(Sx)-FS-Z (SEQ ID NO: 2484), X-KAKKTKKRPQRATS*N-C(Sx)-FA-Z (SEQ ID NO: 2485), X-KAKKTKKRPQRATS*N-C(Sx)-FS-Z (SEQ ID NO: 2486), X-KAKKTKKRPQRATSD-C(Sx)-FS*-Z (SEQ ID NO: 2487), X-KAKKTKKRPQRATSN-C(Sx)-FS*-Z (SEQ ID NO: 2488), X-KAKKVKKRPQRADS*D-C(Sx)-FA-Z (SEQ ID NO: 2489), X-KAKKVKKRPQRADS*D-C(Sx)-FS-Z (SEQ ID NO: 2490), X-KAKKVKKRPQRADS*N-C(Sx)-FA-Z (SEQ ID NO: 2491), X-KAKKVKKRPQRADS*N-C(Sx)-FS-Z (SEQ ID NO: 2492), X-KAKKVKKRPQRADSD-C(Sx)-FS*-Z (SEQ ID NO: 2493), X-KAKKVKKRPQRADSN-C(Sx)-FS*-Z (SEQ ID NO: 2494), X-KAKKVKKRPQRAES*D-C(Sx)-FA-Z (SEQ ID NO: 2495), X-KAKKVKKRPQRAES*D-C(Sx)-FS-Z (SEQ ID NO: 2496), X-KAKKVKKRPQRAES*N-C(Sx)-FA-Z (SEQ ID NO: 2497), X-KAKKVKKRPQRAES*N-C(Sx)-FS-Z (SEQ ID NO: 2498), X-KAKKVKKRPQRAESD-C(Sx)-FS*-Z (SEQ ID NO: 2499), X-KAKKVKKRPQRAESN-C(Sx)-FS*-Z (SEQ ID NO: 2500), X-KAKKVKKRPQRAHS*D-C(Sx)-FA-Z (SEQ ID NO: 2501), X-KAKKVKKRPQRAHS*D-C(Sx)-FS-Z (SEQ ID NO: 2502), X-KAKKVKKRPQRAHS*N-C(Sx)-FA-Z (SEQ ID NO: 2503), and X-KAKKVKKRPQRAHS*N-C(Sx)-FS-Z (SEQ ID NO: 2504); wherein X— is H or acetyl; and Z is OH or NH2.

In some embodiments, the metal-binding compound of the present invention is selected from the group consisting of: X-KAKKVKKRPQRAHSD-C(Sx)-FS*-Z (SEQ ID NO: 2505), X-KAKKVKKRPQRAHSN-C(Sx)-FS*-Z (SEQ ID NO: 2506), X-KAKKVKKRPQRATS*D-C(Sx)-FA-Z (SEQ ID NO: 2507), X-KAKKVKKRPQRATS*D-C(Sx)-FS-Z (SEQ ID NO: 2508), X-KAKKVKKRPQRATS*N-C(Sx)-FA-Z (SEQ ID NO: 2509), X-KAKKVKKRPQRATS*N-C(Sx)-FS-Z (SEQ ID NO: 2510), X-KAKKVKKRPQRATSD-C(Sx)-FS*-Z (SEQ ID NO: 2511), X-KAKKVKKRPQRATSN-C(Sx)-FS*-Z (SEQ ID NO: 2512), X-KAKLTKKRPQRATS*N-C(Sx)-FS-Z (SEQ ID NO: 2513), X-KAKLTKKRPQRATSN-C(Sx)-FS*-Z (SEQ ID NO: 2514), X-KAKNTKKRPQRATS*N-C(Sx)-FS-Z (SEQ ID NO: 2515), X-KAKNTKKRPQRATSN-C(Sx)-FS*-Z (SEQ ID NO: 2516), X-KAKPTKKRPQRATS*N-C(Sx)-FS-Z (SEQ ID NO: 2517), X-KAKPTKKRPQRATSN-C(Sx)-FS*-Z (SEQ ID NO: 2518), X-KAKRTKKRPQRATS*N-C(Sx)-FS-Z (SEQ ID NO: 2519), X-KAKRTKKRPQRATSN-C(Sx)-FS*-Z (SEQ ID NO: 2520), X-KAKT[Nle]KKRPQRATS*N-C(Sx)-FS-Z (SEQ ID NO: 2521), X-KAKT[Nle]KKRPQRATSN-C(Sx)-FS*-Z (SEQ ID NO: 2522), X-KAKTDKKRPQRATS*N-C(Sx)-FS-Z (SEQ ID NO: 2523), X-KAKTDKKRPQRATSN-C(Sx)-FS*-Z (SEQ ID NO: 2524), X-KAKTEKKRPQRATS*N-C(Sx)-FS-Z (SEQ ID NO: 2525), X-KAKTEKKRPQRATSN-C(Sx)-FS*-Z (SEQ ID NO: 2526), X-KAKTFKKRPQRATS*N-C(Sx)-FS-Z (SEQ ID NO: 2527), X-KAKTFKKRPQRATSN-C(Sx)-FS*-Z (SEQ ID NO: 2528), X-KAKTGKKRPQRATS*N-C(Sx)-FS-Z (SEQ ID NO: 2529), X-KAKTGKKRPQRATSN-C(Sx)-FS*-Z (SEQ ID NO: 2530), X-KAKTHKKRPQRATS*N-C(Sx)-FS-Z (SEQ ID NO: 2531), X-KAKTHKKRPQRATSN-C(Sx)-FS*-Z (SEQ ID NO: 2532), X-KAKTIKKRPQRATS*N-C(Sx)-FS-Z (SEQ ID NO: 2533), X-KAKTIKKRPQRATSN-C(Sx)-FS*-Z (SEQ ID NO: 2534), X-KAKTKKKRPQRADS*D-C(Sx)-FA-Z (SEQ ID NO: 2535), X-KAKTKKKRPQRADS*D-C(Sx)-FS-Z (SEQ ID NO: 2536), X-KAKTKKKRPQRADS*N-C(Sx)-FA-Z (SEQ ID NO: 2537), X-KAKTKKKRPQRADS*N-C(Sx)-FS-Z (SEQ ID NO: 2538), X-KAKTKKKRPQRADSD-C(Sx)-FS*-Z (SEQ ID NO: 2539), X-KAKTKKKRPQRADSN-C(Sx)-FS*-Z (SEQ ID NO: 2540), X-KAKTKKKRPQRAES*D-C(Sx)-FA-Z (SEQ ID NO: 2541), X-KAKTKKKRPQRAES*D-C(Sx)-FS-Z (SEQ ID NO: 2542), X-KAKTKKKRPQRAES*N-C(Sx)-FA-Z (SEQ ID NO: 2543), X-KAKTKKKRPQRAES*N-C(Sx)-FS-Z (SEQ ID NO: 2544), X-KAKTKKKRPQRAESD-C(Sx)-FS*-Z (SEQ ID NO: 2545), X-KAKTKKKRPQRAESN-C(Sx)-FS*-Z (SEQ ID NO: 2546), X-KAKTKKKRPQRAHS*D-C(Sx)-FA-Z (SEQ ID NO: 2547), X-KAKTKKKRPQRAHS*D-C(Sx)-FS-Z (SEQ ID NO: 2548), X-KAKTKKKRPQRAHS*N-C(Sx)-FA-Z (SEQ ID NO: 2549), X-KAKTKKKRPQRAHS*N-C(Sx)-FS-Z (SEQ ID NO: 2550), X-KAKTKKKRPQRAHSD-C(Sx)-FS*-Z (SEQ ID NO: 2551), and X-KAKTKKKRPQRAHSN-C(Sx)-FS*-Z (SEQ ID NO: 2552), wherein X is H or acetyl; and Z is OH or NH2.

In some embodiments, the metal-binding compound of the present invention is selected from the group consisting of: X-KAKTKKKRPQRATS*D-C(Sx)-FA-Z (SEQ ID NO: 2553), X-KAKTKKKRPQRATS*D-C(Sx)-FS-Z (SEQ ID NO: 2554), X-KAKTKKKRPQRATS*N-C(Sx)-FA-Z (SEQ ID NO: 2555), X-KAKTKKKRPQRATS*N-C(Sx)-FS-Z (SEQ ID NO: 2556), X-KAKTKKKRPQRATSD-C(Sx)-FS*-Z (SEQ ID NO: 2557), X-KAKTKKKRPQRATSN-C(Sx)-FS*-Z (SEQ ID NO: 2558), X-KAKTLKKRPQRATS*N-C(Sx)-FS-Z (SEQ ID NO: 2559), X-KAKTLKKRPQRATSN-C(Sx)-FS*-Z (SEQ ID NO: 2560), X-KAKTNKKRPQRATS*N-C(Sx)-FS-Z (SEQ ID NO: 2561), X-KAKTNKKRPQRATSN-C(Sx)-FS*-Z (SEQ ID NO: 2562), X-KAKTPKKRPQRATS*N-C(Sx)-FS-Z (SEQ ID NO: 2563), X-KAKTPKKRPQRATSN-C(Sx)-FS*-Z (SEQ ID NO: 2564), X-KAKTRKKRPQRADS*D-C(Sx)-FA-Z (SEQ ID NO: 2565), X-KAKTRKKRPQRADS*D-C(Sx)-FS-Z (SEQ ID NO: 2566), X-KAKTRKKRPQRADS*N-C(Sx)-FA-Z (SEQ ID NO: 2567), X-KAKTRKKRPQRADS*N-C(Sx)-FS-Z (SEQ ID NO: 2568), X-KAKTRKKRPQRADSD-C(Sx)-FS*-Z (SEQ ID NO: 2569), X-KAKTRKKRPQRADSN-C(Sx)-FS*-Z (SEQ ID NO: 2570), X-KAKTRKKRPQRAES*D-C(Sx)-FA-Z (SEQ ID NO: 2571), X-KAKTRKKRPQRAES*D-C(Sx)-FS-Z (SEQ ID NO: 2572), X-KAKTRKKRPQRAES*N-C(Sx)-FA-Z (SEQ ID NO: 2573), X-KAKTRKKRPQRAES*N-C(Sx)-FS-Z (SEQ ID NO: 2574), X-KAKTRKKRPQRAESD-C(Sx)-FS*-Z (SEQ ID NO: 2575), X-KAKTRKKRPQRAESN-C(Sx)-FS*-Z (SEQ ID NO: 2576), X-KAKTRKKRPQRAHS*D-C(Sx)-FA-Z (SEQ ID NO: 2577), X-KAKTRKKRPQRAHS*D-C(Sx)-FS-Z (SEQ ID NO: 2578), X-KAKTRKKRPQRAHS*N-C(Sx)-FA-Z (SEQ ID NO: 2579), X-KAKTRKKRPQRAHS*N-C(Sx)-FS-Z (SEQ ID NO: 2580), X-KAKTRKKRPQRAHSD-C(Sx)-FS*-Z (SEQ ID NO: 2581), X-KAKTRKKRPQRAHSN-C(Sx)-FS*-Z (SEQ ID NO: 2582), X-KAKTRKKRPQRATS*D-C(Sx)-FA-Z (SEQ ID NO: 2583), X-KAKTRKKRPQRATS*D-C(Sx)-FS-Z (SEQ ID NO: 2584), X-KAKTRKKRPQRATS*N-C(Sx)-FA-Z (SEQ ID NO: 2585), X-KAKTRKKRPQRATS*N-C(Sx)-FS-Z (SEQ ID NO: 2586); wherein X— is H or acetyl; and Z is OH or NH2.

In some embodiments, the metal-binding compound of the present invention is selected from the group consisting of: X-KAKTRKKRPQRATSD-C(Sx)-FS*-Z (SEQ ID NO: 2587), X-KAKTRKKRPQRATSN-C(Sx)-FS*-Z (SEQ ID NO: 2588), X-KAKTT[Nle]KRPQRATS*N-C(Sx)-FS-Z (SEQ ID NO: 2589), X-KAKTT[Nle]KRPQRATSN-C(Sx)-FS*-Z (SEQ ID NO: 2590), X-KAKTTDKRPQRATS*N-C(Sx)-FS-Z (SEQ ID NO: 2591), X-KAKTDKRPQRATSN-C(Sx)-FS*-Z (SEQ ID NO: 2592), X-KAKTTEKRPQRATS*N-C(Sx)-FS-Z (SEQ ID NO: 2593), X-KAKTTEKRPQRATSN-C(Sx)-FS*-Z (SEQ ID NO: 2594), X-KAKTIFKRPQRATS*N-C(Sx)-FS-Z (SEQ ID NO: 2595), X-KAKTFKRPQRATSN-C(Sx)-FS*-Z (SEQ ID NO: 2596), X-KAKTTGKRPQRATS*N-C(Sx)-FS-Z (SEQ ID NO: 2597), X-KAKTTGKRPQRATSN-C(Sx)-FS*-Z (SEQ ID NO: 2598), X-KAKITHKRPQRATS*N-C(Sx)-FS-Z (SEQ ID NO: 2599), X-KAKITHKRPQRATSN-C(Sx)-FS*-Z (SEQ ID NO: 2600), X-KAKTTIKRPQRATS*N-C(Sx)-FS-Z (SEQ ID NO: 2601), X-KAKTIKRPQRATSN-C(Sx)-FS*-Z (SEQ ID NO: 2602), X-KAKTTK[Nle]RPQRATS*N-C(Sx)-FS-Z (SEQ ID NO: 2603), X-KAKTTK[Nle]RPQRATSN-C(Sx)-FS*-Z (SEQ ID NO: 2604), X-KAKTTKDRPQRATS*N-C(Sx)-FS-Z (SEQ ID NO: 2605), X-KAKTTKDRPQRATSN-C(Sx)-FS*-Z (SEQ ID NO: 2606), X-KAKTTKERPQRATS*N-C(Sx)-FS-Z (SEQ ID NO: 2607), X-KAKTTKERPQRATSN-C(Sx)-FS*-Z (SEQ ID NO: 2608), X-KAKTTKFRPQRATS*N-C(Sx)-FS-Z (SEQ ID NO: 2609), X-KAKTKFRPQRATSN-C(Sx)-FS*-Z (SEQ ID NO: 2610), X-KAKTTKGRPQRATS*N-C(Sx)-FS-Z (SEQ ID NO: 2611), X-KAKTTKGRPQRATSN-C(Sx)-FS*-Z (SEQ ID NO: 2612), X-KAKTTKHRPQRATS*N-C(Sx)-FS-Z (SEQ ID NO: 2613), X-KAKTTKHRPQRATSN-C(Sx)-FS*-Z (SEQ ID NO: 2614), X-KAKTTKIRPQRATS*N-C(Sx)-FS-Z (SEQ ID NO: 2615), X-KAKTTKIRPQRATSN-C(Sx)-FS*-Z (SEQ ID NO: 2616), X-KAKTTKK[Nle]PQRATS*N-C(Sx)-FS-Z (SEQ ID NO: 2617), X-KAKTTKK[Nle]PQRATSN-C(Sx)-FS*-Z (SEQ ID NO: 2618), X-KAKTTKKDPQRATS*N-C(Sx)-FS-Z (SEQ ID NO: 2619), X-KAKTKKDPQRATSN-C(Sx)-FS*-Z (SEQ ID NO: 2620), X-KAKTTKKEPQRATS*N-C(Sx)-FS-Z (SEQ ID NO: 2621), X-KAKTTKKEPQRATSN-C(Sx)-FS*-Z (SEQ ID NO: 2622), X-KAKTTKKFPQRATS*N-C(Sx)-FS-Z (SEQ ID NO: 2623), X-KAKTTKKFPQRATSN-C(Sx)-FS*-Z (SEQ ID NO: 2624), X-KAKTTKKGPQRATS*N-C(Sx)-FS-Z (SEQ ID NO: 2625), X-KAKTTKKGPQRATSN-C(Sx)-FS*-Z (SEQ ID NO: 2626), X-KAKTTKKHPQRATS*N-C(Sx)-FS-Z (SEQ ID NO: 2627), X-KAKTTKKHPQRATSN-C(Sx)-FS*-Z (SEQ ID NO: 2628), X-KAKTTKKIPQRATS*N-C(Sx)-FS-Z (SEQ ID NO: 2629), X-KAKTTKKIPQRATSN-C(Sx)-FS*-Z (SEQ ID NO: 2630), X-KAKTTKKKPQRATS*N-C(Sx)-FS-Z (SEQ ID NO: 2631), X-KAKTTKKKPQRATSN-C(Sx)-FS*-Z (SEQ ID NO: 2632), X-KAKTTKKLPQRATS*N-C(Sx)-FS-Z (SEQ ID NO: 2633), X-KAKTTKKLPQRATSN-C(Sx)-FS*-Z (SEQ ID NO: 2634), X-KAKTTKKNPQRATS*N-C(Sx)-FS-Z (SEQ ID NO: 2635), X-KAKTTKKNPQRATSN-C(Sx)-FS*-Z (SEQ ID NO: 2636), X-KAKTTKKPPQRATS*N-C(Sx)-FS-Z (SEQ ID NO: 2637), X-KAKTTKKPPQRATSN-C(Sx)-FS*-Z (SEQ ID NO: 2638), X-KAKTTKKR[Nle]QRATS*N-C(Sx)-FS-Z (SEQ ID NO: 2639), X-KAKTTKKR[Nle]QRATSN-C(Sx)-FS*-Z (SEQ ID NO: 2640), X-KAKTTKKRDQRATS*N-C(Sx)-FS-Z (SEQ ID NO: 2641), X-KAKTTKKRDQRATSN-C(Sx)-FS*-Z (SEQ ID NO: 2642), X-KAKTTKKREQRATS*N-C(Sx)-FS-Z (SEQ ID NO: 2643), X-KAKTTKKREQRATSN-C(Sx)-FS*-Z (SEQ ID NO: 2644), X-KAKTTKKRFQRATS*N-C(Sx)-FS-Z (SEQ ID NO: 2645), X-KAKTTKKRFQRATSN-C(Sx)-FS*-Z (SEQ ID NO: 2646), X-KAKTTKKRGQRATS*N-C(Sx)-FS-Z (SEQ ID NO: 2647), X-KAKTTKKRGQRATSN-C(Sx)-FS*-Z (SEQ ID NO: 2648), X-KAKTTKKRHQRATS*N-C(Sx)-FS-Z (SEQ ID NO: 2649), X-KAKTITKKRHQRATSN-C(Sx)-FS*-Z (SEQ ID NO: 2650), X-KAKTTKKRIQRATS*N-C(Sx)-FS-Z (SEQ ID NO: 2651), X-KAKTTKKRIQRATSN-C(Sx)-FS*-Z (SEQ ID NO: 2652), X-KAKTTKKRKQRATS*N-C(Sx)-FS-Z (SEQ ID NO: 2653), X-KAKTrKKRKQRATSN-C(Sx)-FS*-Z (SEQ ID NO: 2654), X-KAKTTKKRLQRATS*N-C(Sx)-FS-Z (SEQ ID NO: 2655), X-KAKTTKKRLQRATSN-C(Sx)-FS*-Z (SEQ ID NO: 2656), X-KAKTTKKRNQRATS*N-C(Sx)-FS-Z (SEQ ID NO: 2657), X-KAKTrKKRNQRATSN-C(Sx)-FS*-Z (SEQ ID NO: 2658), X-KAKTTKKRP[Nle]RATS*N-C(Sx)-FS-Z (SEQ ID NO: 2659), X-KAKTTKKRP[Nle]RATSN-C(Sx)-FS*-Z (SEQ ID NO: 2660), and X-KAKTTKKRPDRATS*N-C(Sx)-FS-Z (SEQ ID NO: 2661); wherein X is H or acetyl; and Z is OH or NH2.

In some embodiments, the metal-binding compound of the present invention is selected from the group consisting of: X-KAKTTKKRPDRATSN-C(Sx)-FS*-Z (SEQ ID NO: 2662), X-KAKTTKKRPERATS*N-C(Sx)-FS-Z (SEQ ID NO: 2663), X-KAKTTKKRPERATSN-C(Sx)-FS*-Z (SEQ ID NO: 2664), X-KAKTTKKRPFRATS*N-C(Sx)-FS-Z (SEQ ID NO: 2665), X-KAKTIKKRPFRATSN-C(Sx)-FS*-Z (SEQ ID NO: 2666), X-KAKTTKKRPGRATS*N-C(Sx)-FS-Z (SEQ ID NO: 2667), X-KAKTITKKRPGRATSN-C(Sx)-FS*-Z (SEQ ID NO: 2668), X-KAKTTKKRPHRATS*N-C(Sx)-FS-Z (SEQ ID NO: 2669), X-KAKTIKKRPHRATSN-C(Sx)-FS*-Z (SEQ ID NO: 2670), X-KAKTTKKRPIRATS*N-C(Sx)-FS-Z (SEQ ID NO: 2671), X-KAKTTKKRPIRATSN-C(Sx)-FS*-Z (SEQ ID NO: 2672), X-KAKTTKKRPKRATS*N-C(Sx)-FS-Z (SEQ ID NO: 2673), X-KAKTIKKRPKRATSN-C(Sx)-FS*-Z (SEQ ID NO: 2674), X-KAKTTKKRPLRATS*N-C(Sx)-FS-Z (SEQ ID NO: 2675), X-KAKTTKKRPLRATSN-C(Sx)-FS*-Z (SEQ ID NO: 2676), X-KAKTTKKRPNRATS*N-C(Sx)-FS-Z (SEQ ID NO: 2677), X-KAKTIKKRPNRATSN-C(Sx)-FS*-Z (SEQ ID NO: 2678), X-KAKTTKKRPPRATS*N-C(Sx)-FS-Z (SEQ ID NO: 2679), X-KAKTTKKRPPRATSN-C(Sx)-FS*-Z (SEQ ID NO: 2680), X-KAKTTKKRPQ[Nle]ATS*N-C(Sx)-FS-Z (SEQ ID NO: 2681), X-KAKTTKKRPQ[Nle]ATSN-C(Sx)-FS*-Z (SEQ ID NO: 2682), X-KAKTTKKRPQDATS*N-C(Sx)-FS-Z (SEQ ID NO: 2683), X-KAKTITKKRPQDATSN-C(Sx)-FS*-Z (SEQ ID NO: 2684), X-KAKTTKKRPQEATS*N-C(Sx)-FS-Z (SEQ ID NO: 2685), X-KAKTIKKRPQEATSN-C(Sx)-FS*-Z (SEQ ID NO: 2686), X-KAKTTKKRPQFATS*N-C(Sx)-FS-Z (SEQ ID NO: 2687), X-KAKTTKKRPQFATSN-C(Sx)-FS*-Z (SEQ ID NO: 2688), X-KAKTTKKRPQGATS*N-C(Sx)-FS-Z (SEQ ID NO: 2689), X-KAKTTKKRPQGATSN-C(Sx)-FS*-Z (SEQ ID NO: 2690), X-KAKTTKKRPQHATS*N-C(Sx)-FS-Z (SEQ ID NO: 2691), X-KAKTTKKRPQHATSN-C(Sx)-FS*-Z (SEQ ID NO: 2692), X-KAKTKKRPQIATS*N-C(Sx)-FS-Z (SEQ ID NO: 2693), X-KAKTFKKRPQIATSN-C(Sx)-FS*-Z (SEQ ID NO: 2694), X-KAKTTKKRPQKATS*N-C(Sx)-FS-Z (SEQ ID NO: 2695), X-KAKTTKKRPQKATSN-C(Sx)-FS*-Z (SEQ ID NO: 2696), X-KAKTTKKRPQLATS*N-C(Sx)-FS-Z (SEQ ID NO: 2697), X-KAKTFKKRPQLATSN-C(Sx)-FS*-Z (SEQ ID NO: 2698), X-KAKTTKKRPQNATS*N-C(Sx)-FS-Z (SEQ ID NO: 2699), X-KAKTTKKRPQNATSN-C(Sx)-FS*-Z (SEQ ID NO: 2700), X-KAKTTKKRPQPATS*N-C(Sx)-FS-Z (SEQ ID NO: 2701), X-KAKTTKKRPQPATSN-C(Sx)-FS*-Z (SEQ ID NO: 2702); wherein X— is H or acetyl; and Z is OH or NH2.

In some embodiments, the metal-binding compound of the present invention is selected from the group consisting of: X-KAKTFKKRPQR[Nle]TS*N-C(Sx)-FS-Z (SEQ ID NO: 2703), X-KAKTFKKRPQR[Nle]TSN-C(Sx)-FS*-Z (SEQ ID NO: 2704), X-KAKTTKKRPQRA[Nle]S*N-C(Sx)-FS-Z (SEQ ID NO: 2705), X-KAKTTKKRPQRA[Nle]SN-C(Sx)-FS*-Z (SEQ ID NO: 2706), X-KAKTTKKRPQRADS*D-C(Sx)-FA-Z (SEQ ID NO: 2707), X-KAKTFKKRPQRADS*D-C(Sx)-FS-Z (SEQ ID NO: 2708), X-KAKTFKKRPQRADS*N-C(Sx)-FA-Z (SEQ ID NO: 2709), X-KAKTFKKRPQRADS*N-C(Sx)-FS-Z (SEQ ID NO: 2710), X-KAKTTKKRPQRADSD-C(Sx)-FS*-Z (SEQ ID NO: 2711), X-KAKTKKRPQRADSN-C(Sx)-FS*-Z (SEQ ID NO: 2712), X-KAKTTKKRPQRAES*D-C(Sx)-FA-Z (SEQ ID NO: 2713), X-KAKTFKKRPQRAES*D-C(Sx)-FS-Z (SEQ ID NO: 2714), X-KAKTTKKRPQRAES*N-C(Sx)-FA-Z (SEQ ID NO: 2715), X-KAKTTKKRPQRAES*N-C(Sx)-FS-Z (SEQ ID NO: 2716), X-KAKTFKKRPQRAESD-C(Sx)-FS*-Z (SEQ ID NO: 2717), X-KAKTFKKRPQRAESN-C(Sx)-FS*-Z (SEQ ID NO: 2718), X-KAKTTKKRPQRAFS*N-C(Sx)-FS-Z (SEQ ID NO: 2719), X-KAKTFKKRPQRAFSN-C(Sx)-FS*-Z (SEQ ID NO: 2720), X-KAKTTKKRPQRAGS*N-C(Sx)-FS-Z (SEQ ID NO: 2721), X-KAKTFKKRPQRAGSN-C(Sx)-FS*-Z (SEQ ID NO: 2722), X-KAKTTKKRPQRAHS*D-C(Sx)-FA-Z (SEQ ID NO: 2723), X-KAKTFKKRPQRAHS*D-C(Sx)-FS-Z (SEQ ID NO: 2724), X-KAKTFKKRPQRAHS*N-C(Sx)-FA-Z (SEQ ID NO: 2725), X-KAKTFKKRPQRAHS*N-C(Sx)-FS-Z (SEQ ID NO: 2726), X-KAKTTKKRPQRAHSD-C(Sx)-FS*-Z (SEQ ID NO: 2727), X-KAKTKKRPQRAHSN-C(Sx)-FS*-Z (SEQ ID NO: 2728), X-KAKTTKKRPQRAIS*N-C(Sx)-FS-Z (SEQ ID NO: 2729), X-KAKTTKKRPQRAISN-C(Sx)-FS*-Z (SEQ ID NO: 2730), X-KAKTTKKRPQRAKS*N-C(Sx)-FS-Z (SEQ ID NO: 2731), X-KAKTTKKRPQRAKSN-C(Sx)-FS*-Z (SEQ ID NO: 2732), X-KAKTTKKRPQRALS*N-C(Sx)-FS-Z (SEQ ID NO: 2733), X-KAKTTKKRPQRALSN-C(Sx)-FS*-Z (SEQ ID NO: 2734), X-KAKTTKKRPQRANS*N-C(Sx)-FS-Z (SEQ ID NO: 2735), X-KAKTTKKRPQRANSN-C(Sx)-FS*-Z (SEQ ID NO: 2736), X-KAKTTKKRPQRAPS*N-C(Sx)-FS-Z (SEQ ID NO: 2737), X-KAKTTKKRPQRAPSN-C(Sx)-FS*-Z (SEQ ID NO: 2738), X-KAKTTKKRPQRARS*N-C(Sx)-FS-Z (SEQ ID NO: 2739), X-KAKTTKKRPQRARSN-C(Sx)-FS*-Z (SEQ ID NO: 2740); wherein X— is H or acetyl; and Z is OH or NH2.

In some embodiments, the metal-binding compound of the present invention is selected from the group consisting of: X-KAKTTKKRPQRAT*S-C(Sx)-VFAMF-Z (SEQ ID NO: 2741), X-KAKTTKKRPQRAT*S-C(Sx)-VFS-Z (SEQ ID NO: 2742), X-KAKTTKKRPQRATS*[Nle]-C(Sx)-FS-Z (SEQ ID NO: 2743), X-KAKTTKKRPQRATS*D-C(Sx)-FA-Z (SEQ ID NO: 2744), X-KAKTTKKRPQRATS*D-C(Sx)-FS-Z (SEQ ID NO: 2745), X-KAKTTKKRPQRATS*E-C(Sx)-FS-Z (SEQ ID NO: 2746), X-KAKTTKKRPQRATS*F-C(Sx)-FS-Z (SEQ ID NO: 2747), X-KAKTTKKRPQRATS*G-C(Sx)-FS-Z (SEQ ID NO: 2748), X-KAKTTKKRPQRATS*H-C(Sx)-FS-Z (SEQ ID NO: 2749), X-KAKTTKKRPQRATS*I-C(Sx)-FS-Z (SEQ ID NO: 2750), X-KAKTTKKRPQRATS*K-C(Sx)-FS-Z (SEQ ID NO: 2751), X-KAKTTKKRPQRATS*L-C(Sx)-FS-Z (SEQ ID NO: 2752), X-KAKTTKKRPQRATS*N-C(Sx)-[Nle]S-Z (SEQ ID NO: 2753), X-KAKTTKKRPQRATS*N-C(Sx)-DS-Z (SEQ ID NO: 2754), X-KAKTTKKRPQRATS*N-C(Sx)-ES-Z (SEQ ID NO: 2755), X-KAKTTKKRPQRATS*N-C(Sx)-F[Nle]-Z (SEQ ID NO: 2756), X-KAKTTKKRPQRATS*N-C(Sx)-FA-Z (SEQ ID NO: 2757), X-KAKTTKKRPQRATS*N-C(Sx)-FD-Z (SEQ ID NO: 2758), X-KAKTTKKRPQRATS*N-C(Sx)-FE-Z (SEQ ID NO: 2759), X-KAKTTKKRPQRATS*N-C(Sx)-FF-Z (SEQ ID NO: 2760), X-KAKTTKKRPQRATS*N-C(Sx)-FG-Z (SEQ ID NO: 2761), X-KAKTTKKRPQRATS*N-C(Sx)-FH-Z (SEQ ID NO: 2762), X-KAKTTKKRPQRATS*N-C(Sx)-FI-Z (SEQ ID NO: 2763), X-KAKTTKKRPQRATS*N-C(Sx)-FK-Z (SEQ ID NO: 2764), X-KAKTTKKRPQRATS*N-C(Sx)-FL-Z (SEQ ID NO: 2765), X-KAKTTKKRPQRATS*N-C(Sx)-FN-Z (SEQ ID NO: 2766), X-KAKTTKKRPQRATS*N-C(Sx)-FP-Z (SEQ ID NO: 2767), X-KAKTTKKRPQRATS*N-C(Sx)-FR-Z (SEQ ID NO: 2768), X-KAKTTKKRPQRATS*N-C(Sx)-FS-Z (SEQ ID NO: 999), X-KAKTTKKRPQRATS*N-C(Sx)-FV-Z (SEQ ID NO: 2769), X-KAKTTKKRPQRATS*N-C(Sx)-FW-Z (SEQ ID NO: 2770), X-KAKTTKKRPQRATS*N-C(Sx)-F-Z (SEQ ID NO: 2771), X-KAKTTKKRPQRATS*N-C(Sx)-GS-Z (SEQ ID NO: 2772), X-KAKTTKKRPQRATS*N-C(Sx)-HS-Z (SEQ ID NO: 2773), X-KAKTTKKRPQRATS*N-C(Sx)-IS-Z (SEQ ID NO: 2774), X-KAKTTKKRPQRATS*N-C(Sx)-KS-Z (SEQ ID NO: 2775), X-KAKTTKKRPQRATS*N-C(Sx)-LS-Z (SEQ ID NO: 2776), X-KAKTTKKRPQRATS*N-C(Sx)-NS-Z (SEQ ID NO: 2777), X-KAKTTKKRPQRATS*N-C(Sx)-PS-Z (SEQ ID NO: 2778), X-KAKTTKKRPQRATS*N-C(Sx)-RS-Z (SEQ ID NO: 2779), X-KAKTTKKRPQRATS*N-C(Sx)-VS-Z (SEQ ID NO: 2780), X-KAKTTKKRPQRATS*N-C(Sx)-WS-Z (SEQ ID NO: 2781), X-KAKTTKKRPQRATS*P-C(Sx)-FS-Z (SEQ ID NO: 2782), X-KAKTTKKRPQRATS*R-C(Sx)-FS-Z (SEQ ID NO: 2783), X-KAKTTKKRPQRATS*V-C(Sx)-FS-Z (SEQ ID NO: 2784), X-KAKTTKKRPQRATS*W-C(Sx)-FS-Z (SEQ ID NO: 2785), X-KAKTTKKRPQRATS[Nle]-C(Sx)-FS*-Z (SEQ ID NO: 2786), X-KAKTTKKRPQRATSD-C(Sx)-FS*-Z (SEQ ID NO: 2787), X-KAKTTKKRPQRATSE-C(Sx)-FS*-Z (SEQ ID NO: 2788), X-KAKTTKKRPQRATSF-C(Sx)-FS*-Z (SEQ ID NO: 2789), X-KAKTTKKRPQRATSG-C(Sx)-FS*-Z (SEQ ID NO: 2790), X-KAKTTKKRPQRATSH-C(Sx)-FS*-Z (SEQ ID NO: 2791), X-KAKTTKKRPQRATSI-C(Sx)-FS*-Z (SEQ ID NO: 2792), X-KAKTTKKRPQRATSK-C(Sx)-FS*-Z (SEQ ID NO: 2793), X-KAKTTKKRPQRATSL-C(Sx)-FS*-Z (SEQ ID NO: 2794), X-KAKTTKKRPQRATSN-C(Sx)-DS*-Z (SEQ ID NO: 2795), X-KAKTTKKRPQRATSN-C(Sx)-ES*-Z (SEQ ID NO: 2796), X-KAKTTKKRPQRATSN-C(Sx)-FS*-Z (SEQ ID NO: 2797), X-KAKTTKKRPQRATSN-C(Sx)-GS*-Z (SEQ ID NO: 2798), X-KAKTTKKRPQRATSN-C(Sx)-HS*-Z (SEQ ID NO: 2799), X-KAKTTKKRPQRATSN-C(Sx)-IS*-Z (SEQ ID NO: 2800), X-KAKTKKRPQRATSN-C(Sx)-KS*-Z (SEQ ID NO: 2801), X-KAKTTKKRPQRATSN-C(Sx)-LS*-Z (SEQ ID NO: 2802), X-KAKTTKKRPQRATSN-C(Sx)-NS*-Z (SEQ ID NO: 2803), X-KAKTTKKRPQRATSN-C(Sx)-PS*-Z (SEQ ID NO: 2804), X-KAKTKKRPQRATSN-C(Sx)-RS*-Z (SEQ ID NO: 2805), X-KAKTTKKRPQRATSN-C(Sx)-VS*-Z (SEQ ID NO: 2806), X-KAKTTKKRPQRATSN-C(Sx)-WS*-Z (SEQ ID NO: 2807), X-KAKTTKKRPQRATSP-C(Sx)-FS*-Z (SEQ ID NO: 2808), X-KAKTTKKRPQRATSR-C(Sx)-FS*-Z (SEQ ID NO: 2809), X-KAKTTKKRPQRATSV-C(Sx)-FS*-Z (SEQ ID NO: 2810), X-KAKTTKKRPQRATSW-C(Sx)-FS*-Z (SEQ ID NO: 2811), X-KAKTTKKRPQRAVS*N-C(Sx)-FS-Z (SEQ ID NO: 2812), X-KAKTITKKRPQRAVSN-C(Sx)-FS*-Z (SEQ ID NO: 2813), X-KAKTTKKRPQRAWS*N-C(Sx)-FS-Z (SEQ ID NO: 2814), X-KAKTTKKRPQRAWSN-C(Sx)-FS*-Z (SEQ ID NO: 2815), X-KAKTTKKRPQRDTS*N-C(Sx)-FS-Z (SEQ ID NO: 2816), X-KAKTTKKRPQRDTSN-C(Sx)-FS*-Z (SEQ ID NO: 2817), X-KAKTTKKRPQRETS*N-C(Sx)-FS-Z (SEQ ID NO: 2818), X-KAKTTKKRPQRETSN-C(Sx)-FS*-Z (SEQ ID NO: 2819), and X-KAKTTKKRPQRFTS*N-C(Sx)-FS-Z (SEQ ID NO: 2820); wherein X is H or acetyl; and Z is OH or NH2.

In some embodiments, the metal-binding compound of the present invention is selected from the group consisting of: X-KAKTTKKRPQRFTSN-C(Sx)-FS*-Z (SEQ ID NO: 2821), X-KAKTTKKRPQRGTS*N-C(Sx)-FS-Z (SEQ ID NO: 2822), X-KAKTIKKRPQRGTSN-C(Sx)-FS*-Z (SEQ ID NO: 2823), X-KAKTTKKRPQRHTS*N-C(Sx)-FS-Z (SEQ ID NO: 2824), X-KAKTIKKRPQRHTSN-C(Sx)-FS*-Z (SEQ ID NO: 2825), X-KAKTTKKRPQRITS*N-C(Sx)-FS-Z (SEQ ID NO: 2826), X-KAKTTKKRPQRITSN-C(Sx)-FS*-Z (SEQ ID NO: 2827), X-KAKTTKKRPQRKTS*N-C(Sx)-FS-Z (SEQ ID NO: 2828), X-KAKTIKKRPQRKTSN-C(Sx)-FS*-Z (SEQ ID NO: 2829), X-KAKTTKKRPQRLTS*N-C(Sx)-FS-Z (SEQ ID NO: 2830), X-KAKTTKKRPQRLTSN-C(Sx)-FS*-Z (SEQ ID NO: 2831), X-KAKTTKKRPQRNTS*N-C(Sx)-FS-Z (SEQ ID NO: 2832), X-KAKTIKKRPQRNTSN-C(Sx)-FS*-Z (SEQ ID NO: 2833), X-KAKTTKKRPQRPTS*N-C(Sx)-FS-Z (SEQ ID NO: 2834), X-KAKTTKKRPQRPTSN-C(Sx)-FS*-Z (SEQ ID NO: 2835), X-KAKTTKKRPQRRTS*N-C(Sx)-FS-Z (SEQ ID NO: 2836), X-KAKTIKKRPQRRTSN-C(Sx)-FS*-Z (SEQ ID NO: 2837), X-KAKTTKKRPQRVTS*N-C(Sx)-FS-Z (SEQ ID NO: 2838), X-KAKTTKKRPQRVTSN-C(Sx)-FS*-Z (SEQ ID NO: 2839), X-KAKTTKKRPQRWTS*N-C(Sx)-FS-Z (SEQ ID NO: 2840), X-KAKTTKKRPQRWTSN-C(Sx)-FS*-Z (SEQ ID NO: 2841), X-KAKTTKKRPQVATS*N-C(Sx)-FS-Z (SEQ ID NO: 2842), X-KAKTTKKRPQVATSN-C(Sx)-FS*-Z (SEQ ID NO: 2843), X-KAKTTKKRPQWATS*N-C(Sx)-FS-Z (SEQ ID NO: 2844), X-KAKTTKKRPQWATSN-C(Sx)-FS*-Z (SEQ ID NO: 2845), X-KAKTTKKRPRRATS*N-C(Sx)-FS-Z (SEQ ID NO: 2846), X-KAKTITKKRPRRATSN-C(Sx)-FS*-Z (SEQ ID NO: 2847), X-KAKTTKKRPVRATS*N-C(Sx)-FS-Z (SEQ ID NO: 2848), X-KAKTIKKRPVRATSN-C(Sx)-FS*-Z (SEQ ID NO: 2849), X-KAKTTKKRPWRATS*N-C(Sx)-FS-Z (SEQ ID NO: 2850), X-KAKTTKKRPWRATSN-C(Sx)-FS*-Z (SEQ ID NO: 2851), X-KAKTTKKRRQRATS*N-C(Sx)-FS-Z (SEQ ID NO: 2852), X-KAKTTKKRRQRATSN-C(Sx)-FS*-Z (SEQ ID NO: 2853), X-KAKTTKKRVQRATS*N-C(Sx)-FS-Z (SEQ ID NO: 2854), X-KAKTKKRVQRATSN-C(Sx)-FS*-Z (SEQ ID NO: 2855), X-KAKTTKKRWQRATS*N-C(Sx)-FS-Z (SEQ ID NO: 2856), X-KAKTTKKRWQRATSN-C(Sx)-FS*-Z (SEQ ID NO: 2857), X-KAKTTKKVPQRATS*N-C(Sx)-FS-Z (SEQ ID NO: 2858), X-KAKTTKKVPQRATSN-C(Sx)-FS*-Z (SEQ ID NO: 2859), X-KAKTTKKWPQRATS*N-C(Sx)-FS-Z (SEQ ID NO: 2860), X-KAKTTKKWPQRATSN-C(Sx)-FS*-Z (SEQ ID NO: 2861), X-KAKTTKLRPQRATS*N-C(Sx)-FS-Z (SEQ ID NO: 2862), X-KAKTTKLRPQRATSN-C(Sx)-FS*-Z (SEQ ID NO: 2863), X-KAKTTKNRPQRATS*N-C(Sx)-FS-Z (SEQ ID NO: 2864), X-KAKTTKNRPQRATSN-C(Sx)-FS*-Z (SEQ ID NO: 2865), X-KAKTTKPRPQRATS*N-C(Sx)-FS-Z (SEQ ID NO: 2866), X-KAKTTKPRPQRATSN-C(Sx)-FS*-Z (SEQ ID NO: 2867), X-KAKTTKRRPQRATS*N-C(Sx)-FS-Z (SEQ ID NO: 2868), X-KAKTTKRRPQRATSN-C(Sx)-FS*-Z (SEQ ID NO: 2869), X-KAKTTKVRPQRATS*N-C(Sx)-FS-Z (SEQ ID NO: 2870), X-KAKTTKVRPQRATSN-C(Sx)-FS*-Z (SEQ ID NO: 2871), X-KAKTTKWRPQRATS*N-C(Sx)-FS-Z (SEQ ID NO: 2872), X-KAKTTKWRPQRATSN-C(Sx)-FS*-Z (SEQ ID NO: 2873), X-KAKTTLKRPQRATS*N-C(Sx)-FS-Z (SEQ ID NO: 2874), X-KAKTITLKRPQRATSN-C(Sx)-FS*-Z (SEQ ID NO: 2875), X-KAKTTNKRPQRATS*N-C(Sx)-FS-Z (SEQ ID NO: 2876), X-KAKTTNKRPQRATSN-C(Sx)-FS*-Z (SEQ ID NO: 2877), X-KAKTTPKRPQRATS*N-C(Sx)-FS-Z (SEQ ID NO: 2878), X-KAKTITPKRPQRATSN-C(Sx)-FS*-Z (SEQ ID NO: 2879), X-KAKTTRKRPQRATS*N-C(Sx)-FS-Z (SEQ ID NO: 2880), X-KAKTTRKRPQRATSN-C(Sx)-FS*-Z (SEQ ID NO: 2881), X-KAKTTVKRPQRATS*N-C(Sx)-FS-Z (SEQ ID NO: 2882), X-KAKTTVKRPQRATSN-C(Sx)-FS*-Z (SEQ ID NO: 2883), X-KAKTTWKRPQRATS*N-C(Sx)-FS-Z (SEQ ID NO: 2884), X-KAKTTWKRPQRATSN-C(Sx)-FS*-Z (SEQ ID NO: 2885); wherein X— is H or acetyl; and Z is OH or NH2.

In some embodiments, the metal-binding compound of the present invention is selected from the group consisting of: X-KAKTVKKRPQRADS*D-C(Sx)-FA-Z (SEQ ID NO: 2886), X-KAKTVKKRPQRADS*D-C(Sx)-FS-Z (SEQ ID NO: 2887), X-KAKTVKKRPQRADS*N-C(Sx)-FA-Z (SEQ ID NO: 2888), X-KAKTVKKRPQRADS*N-C(Sx)-FS-Z (SEQ ID NO: 2889), X-KAKTVKKRPQRADSD-C(Sx)-FS*-Z (SEQ ID NO: 2890), X-KAKTVKKRPQRADSN-C(Sx)-FS*-Z (SEQ ID NO: 2891), X-KAKTVKKRPQRAES*D-C(Sx)-FA-Z (SEQ ID NO: 2892), X-KAKTVKKRPQRAES*D-C(Sx)-FS-Z (SEQ ID NO: 2893), X-KAKTVKKRPQRAES*N-C(Sx)-FA-Z (SEQ ID NO: 2894), X-KAKTVKKRPQRAES*N-C(Sx)-FS-Z (SEQ ID NO: 2895), X-KAKTVKKRPQRAESD-C(Sx)-FS*-Z (SEQ ID NO: 2896), X-KAKTVKKRPQRAESN-C(Sx)-FS*-Z (SEQ ID NO: 2897), X-KAKTVKKRPQRAHS*D-C(Sx)-FA-Z (SEQ ID NO: 2898), X-KAKTVKKRPQRAHS*D-C(Sx)-FS-Z (SEQ ID NO: 2899), X-KAKTVKKRPQRAHS*N-C(Sx)-FA-Z (SEQ ID NO: 2900), X-KAKTVKKRPQRAHS*N-C(Sx)-FS-Z (SEQ ID NO: 2901), X-KAKTVKKRPQRAHSD-C(Sx)-FS*-Z (SEQ ID NO: 2902), X-KAKTVKKRPQRAHSN-C(Sx)-FS*-Z (SEQ ID NO: 2903), X-KAKTVKKRPQRATS*D-C(Sx)-FA-Z (SEQ ID NO: 2904), X-KAKTVKKRPQRATS*D-C(Sx)-FS-Z (SEQ ID NO: 2905), X-KAKTVKKRPQRATS*N-C(Sx)-FA-Z (SEQ ID NO: 2906), X-KAKTVKKRPQRATS*N-C(Sx)-FS-Z (SEQ ID NO: 2907), X-KAKTVKKRPQRATSD-C(Sx)-FS*-Z (SEQ ID NO: 2908), X-KAKTVKKRPQRATSN-C(Sx)-FS*-Z (SEQ ID NO: 2909), X-KAKTWKKRPQRATS*N-C(Sx)-FS-Z (SEQ ID NO: 2910), X-KAKTWKKRPQRATSN-C(Sx)-FS*-Z (SEQ ID NO: 2911), X-KAKVTKKRPQRATS*N-C(Sx)-FS-Z (SEQ ID NO: 2912), X-KAKVTKKRPQRATSN-C(Sx)-FS*-Z (SEQ ID NO: 2913), X-KAKWTKKRPQRATS*N-C(Sx)-FS-Z (SEQ ID NO: 2914), X-KAKWTKKRPQRATSN-C(Sx)-FS*-Z (SEQ ID NO: 2915), X-KALTTKKRPQRATS*N-C(Sx)-FS-Z (SEQ ID NO: 2916), X-KALTKKRPQRATSN-C(Sx)-FS*-Z (SEQ ID NO: 2917), X-KANPLMA-C(Sx)-GT*LTRRHQNGRF-Z (SEQ ID NO: 2918), X-KANTTKKRPQRATS*N-C(Sx)-FS-Z (SEQ ID NO: 2919), X-KANTTKKRPQRATSN-C(Sx)-FS*-Z (SEQ ID NO: 2920), X-KAPTTKKRPQRATS*N-C(Sx)-FS-Z (SEQ ID NO: 2921), X-KAPTTKKRPQRATSN-C(Sx)-FS*-Z (SEQ ID NO: 2922), X-KARTTKKRPQRATS*N-C(Sx)-FS-Z (SEQ ID NO: 2923), X-KARTTKKRPQRATSN-C(Sx)-FS*-Z (SEQ ID NO: 2924), X-KAVTTKKRPQRATS*N-C(Sx)-FS-Z (SEQ ID NO: 2925), X-KAVTTKKRPQRATSN-C(Sx)-FS*-Z (SEQ ID NO: 2926), X-KAWTTKKRPQRATS*N-C(Sx)-FS-Z (SEQ ID NO: 2927), X-KAWTTKKRPQRATSN-C(Sx)-FS*-Z (SEQ ID NO: 2928), X-K-C(Sx)-DS*LRRKAK-Z (SEQ ID NO: 2929), X-K-C(Sx)-HS*LPRGNK-Z (SEQ ID NO: 2930), X-K-C(Sx)-LS*PGPF-Z (SEQ ID NO: 2931), X-KDEIL-C(Sx)-TT*PISEQKG-Z (SEQ ID NO: 1086), X-KDEILPTT*P-C(Sx)-SEQKG-Z (SEQ ID NO: 1085), X-KDKTFKKRPQRATS*N-C(Sx)-FS-Z (SEQ ID NO: 2932), X-KDKTFKKRPQRATSN-C(Sx)-FS*-Z (SEQ ID NO: 2933), X-KDRPQRAT*S-C(Sx)-VFAMF-Z (SEQ ID NO: 2934), X-KEEQSQIT*S-C(Sx)-VTGQIGWR-Z (SEQ ID NO: 2935), X-KEEQSQITS-C(Sx)-VT*GQIGWR-Z (SEQ ID NO: 2936), X-KEEQSQITSQVT*G-C(Sx)-IGWR-Z (SEQ ID NO: 2937), X-KEEQSQITSQVT*GQ-C(Sx)-GWR-Z (SEQ ID NO: 2938), X-KEHRG-C(Sx)-DS*PDPDTSY-Z (SEQ ID NO: 1147), X-KEKKAVSPL-C(Sx)-LT*TTNSSEG-Z (SEQ ID NO: 2939), X-KEKKAVSPLL-C(Sx)-TT*TNSSEG-Z (SEQ ID NO: 2940), X-KEKKAVSPLLL-C(Sx)-TI*NSSEG-Z (SEQ ID NO: 2941), X-KEKKAVSPLLLT*T-C(Sx)-NSSEG-Z (SEQ ID NO: 2942), X-KEKKAVSPLLLT*T-C(Sx)-SSEG-Z (SEQ ID NO: 2943), and X-KEKKAVSPLLLTI-C(Sx)-NS*SEG-Z (SEQ ID NO: 2944); wherein X is H or acetyl; and Z is OH or NH2.

In some embodiments, the metal-binding compound of the present invention is selected from the group consisting of: X-KEKKAVSPLLLTIT*N-C(Sx)-SEG-Z (SEQ ID NO: 2945), X-KEKKAVSPLLLTIT-C(Sx)-SS*EG-Z (SEQ ID NO: 2946), X-KEKTTKKRPQRATS*N-C(Sx)-FS-Z (SEQ ID NO: 2947), X-KEKTTKKRPQRATSN-C(Sx)-FS*-Z (SEQ ID NO: 2948), X-KERPQRAT*S-C(Sx)-VFAMF-Z (SEQ ID NO: 2949), X-KEVPRRKS*L-C(Sx)-GTPYW[Nle]APE-Z (SEQ ID NO: 2950), X-KEVPRRKSL-C(Sx)-GT*PYW[Nle]APE-Z (SEQ ID NO: 2951), X-KF-C(Sx)-KT*FKWM-Z (SEQ ID NO: 2952), X-KFFKT*F-C(Sx)-WM-Z (SEQ ID NO: 2953), X-KFKTTKKRPQRATS*N-C(Sx)-FS-Z (SEQ ID NO: 2954), X-KFKTTKKRPQRATSN-C(Sx)-FS*-Z (SEQ ID NO: 2955), X-KFRPQRAT*S-C(Sx)-VFAMF-Z (SEQ ID NO: 2956), X-KGKKKKKRPQRADS*D-C(Sx)-FA-Z (SEQ ID NO: 2957), X-KGKKKKKRPQRADS*D-C(Sx)-FS-Z (SEQ ID NO: 2958), X-KGKKKKKRPQRADS*N-C(Sx)-FA-Z (SEQ ID NO: 2959), X-KGKKKKKRPQRADS*N-C(Sx)-FS-Z (SEQ ID NO: 2960), X-KGKKKKKRPQRADSD-C(Sx)-FS*-Z (SEQ ID NO: 2961), X-KGKKKKKRPQRADSN-C(Sx)-FS*-Z (SEQ ID NO: 2962), X-KGKKKKKRPQRAES*D-C(Sx)-FA-Z (SEQ ID NO: 2963), X-KGKKKKKRPQRAES*D-C(Sx)-FS-Z (SEQ ID NO: 2964), X-KGKKKKKRPQRAES*N-C(Sx)-FA-Z (SEQ ID NO: 2965), X-KGKKKKKRPQRAES*N-C(Sx)-FS-Z (SEQ ID NO: 2966), X-KGKKKKKRPQRAESD-C(Sx)-FS*-Z (SEQ ID NO: 2967), X-KGKKKKKRPQRAESN-C(Sx)-FS*-Z (SEQ ID NO: 2968), X-KGKKKKKRPQRAHS*D-C(Sx)-FA-Z (SEQ ID NO: 2969), X-KGKKKKKRPQRAHS*D-C(Sx)-FS-Z (SEQ ID NO: 2970), X-KGKKKKKRPQRAHS*N-C(Sx)-FA-Z (SEQ ID NO: 2971), X-KGKKKKKRPQRAHS*N-C(Sx)-FS-Z (SEQ ID NO: 2972), X-KGKKKKKRPQRAHSD-C(Sx)-FS*-Z (SEQ ID NO: 2973), X-KGKKKKKRPQRAHSN-C(Sx)-FS*-Z (SEQ ID NO: 2974), X-KGKKKKKRPQRATS*D-C(Sx)-FA-Z (SEQ ID NO: 2975), X-KGKKKKKRPQRATS*D-C(Sx)-FS-Z (SEQ ID NO: 2976), X-KGKKKKKRPQRATS*N-C(Sx)-FA-Z (SEQ ID NO: 2977), X-KGKKKKKRPQRATS*N-C(Sx)-FS-Z (SEQ ID NO: 2978), X-KGKKKKKRPQRATSD-C(Sx)-FS*-Z (SEQ ID NO: 2979), X-KGKKKKKRPQRATSN-C(Sx)-FS*-Z (SEQ ID NO: 2980), X-KGKKRKKRPQRADS*D-C(Sx)-FA-Z (SEQ ID NO: 2981), X-KGKKRKKRPQRADS*D-C(Sx)-FS-Z (SEQ ID NO: 2982), X-KGKKRKKRPQRADS*N-C(Sx)-FA-Z (SEQ ID NO: 2983), X-KGKKRKKRPQRADS*N-C(Sx)-FS-Z (SEQ ID NO: 2984), X-KGKKRKKRPQRADSD-C(Sx)-FS*-Z (SEQ ID NO: 2985), X-KGKKRKKRPQRADSN-C(Sx)-FS*-Z (SEQ ID NO: 2986), and X-KGKKRKKRPQRAES*D-C(Sx)-FA-Z (SEQ ID NO: 2987); wherein X is H or acetyl; and Z is OH or NH2.

In some embodiments, the metal-binding compound of the present invention is selected from the group consisting of: X-KGKKRKKRPQRAES*D-C(Sx)-FS-Z (SEQ ID NO: 2988), X-KGKKRKKRPQRAES*N-C(Sx)-FA-Z (SEQ ID NO: 2989), X-KGKKRKKRPQRAES*N-C(Sx)-FS-Z (SEQ ID NO: 2990), X-KGKKRKKRPQRAESD-C(Sx)-FS*-Z (SEQ ID NO: 2991), X-KGKKRKKRPQRAESN-C(Sx)-FS*-Z (SEQ ID NO: 2992), X-KGKKRKKRPQRAHS*D-C(Sx)-FA-Z (SEQ ID NO: 2993), X-KGKKRKKRPQRAHS*D-C(Sx)-FS-Z (SEQ ID NO: 2994), X-KGKKRKKRPQRAHS*N-C(Sx)-FA-Z (SEQ ID NO: 2995), X-KGKKRKKRPQRAHS*N-C(Sx)-FS-Z (SEQ ID NO: 2996), X-KGKKRKKRPQRAHSD-C(Sx)-FS*-Z (SEQ ID NO: 2997), X-KGKKRKKRPQRAHSN-C(Sx)-FS*-Z (SEQ ID NO: 2998), X-KGKKRKKRPQRATS*D-C(Sx)-FA-Z (SEQ ID NO: 2999), X-KGKKRKKRPQRATS*D-C(Sx)-FS-Z (SEQ ID NO: 3000), X-KGKKRKKRPQRATS*N-C(Sx)-FA-Z (SEQ ID NO: 3001), X-KGKKRKKRPQRATS*N-C(Sx)-FS-Z (SEQ ID NO: 3002), X-KGKKRKKRPQRATSD-C(Sx)-FS*-Z (SEQ ID NO: 3003), X-KGKKRKKRPQRATSN-C(Sx)-FS*-Z (SEQ ID NO: 3004), X-KGKKTKKRPQRADS*D-C(Sx)-FA-Z (SEQ ID NO: 3005), X-KGKKTKKRPQRADS*D-C(Sx)-FS-Z (SEQ ID NO: 3006), X-KGKKTKKRPQRADS*N-C(Sx)-FA-Z (SEQ ID NO: 3007), X-KGKKTKKRPQRADS*N-C(Sx)-FS-Z (SEQ ID NO: 3008), X-KGKKTKKRPQRADSD-C(Sx)-FS*-Z (SEQ ID NO: 3009), X-KGKKTKKRPQRADSN-C(Sx)-FS*-Z (SEQ ID NO: 3010), X-KGKKTKKRPQRAES*D-C(Sx)-FA-Z (SEQ ID NO: 3011), X-KGKKTKKRPQRAES*D-C(Sx)-FS-Z (SEQ ID NO: 3012), X-KGKKTKKRPQRAES*N-C(Sx)-FA-Z (SEQ ID NO: 3013), X-KGKKTKKRPQRAES*N-C(Sx)-FS-Z (SEQ ID NO: 3014), X-KGKKTKKRPQRAESD-C(Sx)-FS*-Z (SEQ ID NO: 3015), X-KGKKTKKRPQRAESN-C(Sx)-FS*-Z (SEQ ID NO: 3016), X-KGKKTKKRPQRAHS*D-C(Sx)-FA-Z (SEQ ID NO: 3017), X-KGKKTKKRPQRAHS*D-C(Sx)-FS-Z (SEQ ID NO: 3018), X-KGKKTKKRPQRAHS*N-C(Sx)-FA-Z (SEQ ID NO: 3019), X-KGKKTKKRPQRAHS*N-C(Sx)-FS-Z (SEQ ID NO: 3020), X-KGKKTKKRPQRAHSD-C(Sx)-FS*-Z (SEQ ID NO: 3021), X-KGKKTKKRPQRAHSN-C(Sx)-FS*-Z (SEQ ID NO: 3022), X-KGKKTKKRPQRATS*D-C(Sx)-FA-Z (SEQ ID NO: 3023), X-KGKKTKKRPQRATS*D-C(Sx)-FS-Z (SEQ ID NO: 3024), X-KGKKTKKRPQRATS*N-C(Sx)-FA-Z (SEQ ID NO: 3025), X-KGKKTKKRPQRATS*N-C(Sx)-FS-Z (SEQ ID NO: 3026), X-KGKKTKKRPQRATSD-C(Sx)-FS*-Z (SEQ ID NO: 3027), X-KGKKTKKRPQRATSN-C(Sx)-FS*-Z (SEQ ID NO: 3028), X-KGKKVKKRPQRADS*D-C(Sx)-FA-Z (SEQ ID NO: 3029), X-KGKKVKKRPQRADS*D-C(Sx)-FS-Z (SEQ ID NO: 3030), X-KGKKVKKRPQRADS*N-C(Sx)-FA-Z (SEQ ID NO: 3031), X-KGKKVKKRPQRADS*N-C(Sx)-FS-Z (SEQ ID NO: 3032), X-KGKKVKKRPQRADSD-C(Sx)-FS*-Z (SEQ ID NO: 3033), X-KGKKVKKRPQRADSN-C(Sx)-FS*-Z (SEQ ID NO: 3034), X-KGKKVKKRPQRAES*D-C(Sx)-FA-Z (SEQ ID NO: 3035), X-KGKKVKKRPQRAES*D-C(Sx)-FS-Z (SEQ ID NO: 3036), X-KGKKVKKRPQRAES*N-C(Sx)-FA-Z (SEQ ID NO: 3037), X-KGKKVKKRPQRAES*N-C(Sx)-FS-Z (SEQ ID NO: 3038), X-KGKKVKKRPQRAESD-C(Sx)-FS*-Z (SEQ ID NO: 3039), X-KGKKVKKRPQRAESN-C(Sx)-FS*-Z (SEQ ID NO: 3040), X-KGKKVKKRPQRAHS*D-C(Sx)-FA-Z (SEQ ID NO: 3041), X-KGKKVKKRPQRAHS*D-C(Sx)-FS-Z (SEQ ID NO: 3042), X-KGKKVKKRPQRAHS*N-C(Sx)-FA-Z (SEQ ID NO: 3043), X-KGKKVKKRPQRAHS*N-C(Sx)-FS-Z (SEQ ID NO: 3044), and X-KGKKVKKRPQRAHSD-C(Sx)-FS*-Z (SEQ ID NO: 3045); wherein X is H or acetyl; and Z is OH or NH2.

In some embodiments, the metal-binding compound of the present invention is selected from the group consisting of: X-KGKKVKKRPQRAHSN-C(Sx)-FS*-Z (SEQ ID NO: 3046), X-KGKKVKKRPQRATS*D-C(Sx)-FA-Z (SEQ ID NO: 3047), X-KGKKVKKRPQRATS*D-C(Sx)-FS-Z (SEQ ID NO: 3048), X-KGKKVKKRPQRATS*N-C(Sx)-FA-Z (SEQ ID NO: 3049), X-KGKKVKKRPQRATS*N-C(Sx)-FS-Z (SEQ ID NO: 3050), X-KGKKVKKRPQRATSD-C(Sx)-FS*-Z (SEQ ID NO: 3051), X-KGKKVKKRPQRATSN-C(Sx)-FS*-Z (SEQ ID NO: 3052), X-KGKTKKKRPQRADS*D-C(Sx)-FA-Z (SEQ ID NO: 3053), X-KGKTKKKRPQRADS*D-C(Sx)-FS-Z (SEQ ID NO: 3054), X-KGKTKKKRPQRADS*N-C(Sx)-FA-Z (SEQ ID NO: 3055), X-KGKTKKKRPQRADS*N-C(Sx)-FS-Z (SEQ ID NO: 3056), X-KGKTKKKRPQRADSD-C(Sx)-FS*-Z (SEQ ID NO: 3057), X-KGKTKKKRPQRADSN-C(Sx)-FS*-Z (SEQ ID NO: 3058), X-KGKTKKKRPQRAES*D-C(Sx)-FA-Z (SEQ ID NO: 3059), X-KGKTKKKRPQRAES*D-C(Sx)-FS-Z (SEQ ID NO: 3060), X-KGKTKKKRPQRAES*N-C(Sx)-FA-Z (SEQ ID NO: 3061), X-KGKTKKKRPQRAES*N-C(Sx)-FS-Z (SEQ ID NO: 3062), X-KGKTKKKRPQRAESD-C(Sx)-FS*-Z (SEQ ID NO: 3063), X-KGKTKKKRPQRAESN-C(Sx)-FS*-Z (SEQ ID NO: 3064), X-KGKTKKKRPQRAHS*D-C(Sx)-FA-Z (SEQ ID NO: 3065), X-KGKTKKKRPQRAHS*D-C(Sx)-FS-Z (SEQ ID NO: 3066), X-KGKTKKKRPQRAHS*N-C(Sx)-FA-Z (SEQ ID NO: 3067), X-KGKTKKKRPQRAHS*N-C(Sx)-FS-Z (SEQ ID NO: 3068), X-KGKTKKKRPQRAHSD-C(Sx)-FS*-Z (SEQ ID NO: 3069), X-KGKTKKKRPQRAHSN-C(Sx)-FS*-Z (SEQ ID NO: 3070), X-KGKTKKKRPQRATS*D-C(Sx)-FA-Z (SEQ ID NO: 3071), X-KGKTKKKRPQRATS*D-C(Sx)-FS-Z (SEQ ID NO: 3072), X-KGKTKKKRPQRATS*N-C(Sx)-FA-Z (SEQ ID NO: 3073), X-KGKTKKKRPQRATS*N-C(Sx)-FS-Z (SEQ ID NO: 3074), X-KGKTKKKRPQRATSD-C(Sx)-FS*-Z (SEQ ID NO: 3075), X-KGKTKKKRPQRATSN-C(Sx)-FS*-Z (SEQ ID NO: 3076), X-KGKTRKKRPQRADS*D-C(Sx)-FA-Z (SEQ ID NO: 3077), X-KGKTRKKRPQRADS*D-C(Sx)-FS-Z (SEQ ID NO: 3078), X-KGKTRKKRPQRADS*N-C(Sx)-FA-Z (SEQ ID NO: 3079), X-KGKTRKKRPQRADS*N-C(Sx)-FS-Z (SEQ ID NO: 3080), X-KGKTRKKRPQRADSD-C(Sx)-FS*-Z (SEQ ID NO: 3081), X-KGKTRKKRPQRADSN-C(Sx)-FS*-Z (SEQ ID NO: 3082), X-KGKTRKKRPQRAES*D-C(Sx)-FA-Z (SEQ ID NO: 3083), X-KGKTRKKRPQRAES*D-C(Sx)-FS-Z (SEQ ID NO: 3084), X-KGKTRKKRPQRAES*N-C(Sx)-FA-Z (SEQ ID NO: 3085), X-KGKTRKKRPQRAES*N-C(Sx)-FS-Z (SEQ ID NO: 3086), X-KGKTRKKRPQRAESD-C(Sx)-FS*-Z (SEQ ID NO: 3087), X-KGKTRKKRPQRAESN-C(Sx)-FS*-Z (SEQ ID NO: 3088), X-KGKTRKKRPQRAHS*D-C(Sx)-FA-Z (SEQ ID NO: 3089), X-KGKTRKKRPQRAHS*D-C(Sx)-FS-Z (SEQ ID NO: 3090), X-KGKTRKKRPQRAHS*N-C(Sx)-FA-Z (SEQ ID NO: 3091), X-KGKTRKKRPQRAHS*N-C(Sx)-FS-Z (SEQ ID NO: 3092), X-KGKTRKKRPQRAHSD-C(Sx)-FS*-Z (SEQ ID NO: 3093), X-KGKTRKKRPQRAHSN-C(Sx)-FS*-Z (SEQ ID NO: 3094), X-KGKTRKKRPQRATS*D-C(Sx)-FA-Z (SEQ ID NO: 3095), X-KGKTRKKRPQRATS*D-C(Sx)-FS-Z (SEQ ID NO: 3096), X-KGKTRKKRPQRATS*N-C(Sx)-FA-Z (SEQ ID NO: 3097), X-KGKTRKKRPQRATS*N-C(Sx)-FS-Z (SEQ ID NO: 3098), X-KGKTRKKRPQRATSD-C(Sx)-FS*-Z (SEQ ID NO: 3099), X-KGKTRKKRPQRATSN-C(Sx)-FS*-Z (SEQ ID NO: 3100), X-KGKTITKKRPQRADS*D-C(Sx)-FA-Z (SEQ ID NO: 3101), X-KGKTTKKRPQRADS*D-C(Sx)-FS-Z (SEQ ID NO: 3102), and X-KGKTTKKRPQRADS*N-C(Sx)-FA-Z (SEQ ID NO: 3103); wherein X is H or acetyl; and Z is OH or NH2.

In some embodiments, the metal-binding compound of the present invention is selected from the group consisting of: X-KGKTTKKRPQRADS*N-C(Sx)-FS-Z (SEQ ID NO: 3104), X-KGKTTKKRPQRADSD-C(Sx)-FS*-Z (SEQ ID NO: 3105), X-KGKTTKKRPQRADSN-C(Sx)-FS*-Z (SEQ ID NO: 3106), X-KGKTTKKRPQRAES*D-C(Sx)-FA-Z (SEQ ID NO: 3107), X-KGKTVKKRPQRAES*D-C(Sx)-FS-Z (SEQ ID NO: 3108), X-KGKTTKKRPQRAES*N-C(Sx)-FA-Z (SEQ ID NO: 3109), X-KGKTTKKRPQRAES*N-C(Sx)-FS-Z (SEQ ID NO: 3110), X-KGKTFKKRPQRAESD-C(Sx)-FS*-Z (SEQ ID NO: 3111), X-KGKTFKKRPQRAESN-C(Sx)-FS*-Z (SEQ ID NO: 3112), X-KGKTTKKRPQRAHS*D-C(Sx)-FA-Z (SEQ ID NO: 3113), X-KGKTKKRPQRAHS*D-C(Sx)-FS-Z (SEQ ID NO: 3114), X-KGKTVKKRPQRAHS*N-C(Sx)-FA-Z (SEQ ID NO: 3115), X-KGKTVKKRPQRAHS*N-C(Sx)-FS-Z (SEQ ID NO: 3116), X-KGKTTKKRPQRAHSD-C(Sx)-FS*-Z (SEQ ID NO: 3117), X-KGKTKKRPQRAHSN-C(Sx)-FS*-Z (SEQ ID NO: 3118), X-KGKTTKKRPQRATS*D-C(Sx)-FA-Z (SEQ ID NO: 3119), X-KGKTVKKRPQRATS*D-C(Sx)-FS-Z (SEQ ID NO: 3120), X-KGKTTKKRPQRATS*N-C(Sx)-FA-Z (SEQ ID NO: 3121), X-KGKTTKKRPQRATS*N-C(Sx)-FS-Z (SEQ ID NO: 3122), X-KGKTFKKRPQRATSD-C(Sx)-FS*-Z (SEQ ID NO: 3123), X-KGKTFKKRPQRATSN-C(Sx)-FS*-Z (SEQ ID NO: 3124), X-KGKTVKKRPQRADS*D-C(Sx)-FA-Z (SEQ ID NO: 3125), X-KGKTVKKRPQRADS*D-C(Sx)-FS-Z (SEQ ID NO: 3126), X-KGKTVKKRPQRADS*N-C(Sx)-FA-Z (SEQ ID NO: 3127), X-KGKTVKKRPQRADS*N-C(Sx)-FS-Z (SEQ ID NO: 3128), X-KGKTVKKRPQRADSD-C(Sx)-FS*-Z (SEQ ID NO: 3129), X-KGKTVKKRPQRADSN-C(Sx)-FS*-Z (SEQ ID NO: 3130), X-KGKTVKKRPQRAES*D-C(Sx)-FA-Z (SEQ ID NO: 3131), X-KGKTVKKRPQRAES*D-C(Sx)-FS-Z (SEQ ID NO: 3132), X-KGKTVKKRPQRAES*N-C(Sx)-FA-Z (SEQ ID NO: 3133), X-KGKTVKKRPQRAES*N-C(Sx)-FS-Z (SEQ ID NO: 3134), X-KGKTVKKRPQRAESD-C(Sx)-FS*-Z (SEQ ID NO: 3135), X-KGKTVKKRPQRAESN-C(Sx)-FS*-Z (SEQ ID NO: 3136), X-KGKTVKKRPQRAHS*D-C(Sx)-FA-Z (SEQ ID NO: 3137), X-KGKTVKKRPQRAHS*D-C(Sx)-FS-Z (SEQ ID NO: 3138), X-KGKTVKKRPQRAHS*N-C(Sx)-FA-Z (SEQ ID NO: 3139), X-KGKTVKKRPQRAHS*N-C(Sx)-FS-Z (SEQ ID NO: 3140), X-KGKTVKKRPQRAHSD-C(Sx)-FS*-Z (SEQ ID NO: 3141), X-KGKTVKKRPQRAHSN-C(Sx)-FS*-Z (SEQ ID NO: 3142), X-KGKTVKKRPQRATS*D-C(Sx)-FA-Z (SEQ ID NO: 3143), X-KGKTVKKRPQRATS*D-C(Sx)-FS-Z (SEQ ID NO: 3144), X-KGKTVKKRPQRATS*N-C(Sx)-FA-Z (SEQ ID NO: 3145), X-KGKTVKKRPQRATS*N-C(Sx)-FS-Z (SEQ ID NO: 3146), X-KGKTVKKRPQRATSD-C(Sx)-FS*-Z (SEQ ID NO: 3147), X-KGKTVKKRPQRATSN-C(Sx)-FS*-Z (SEQ ID NO: 3148), X-KGRPQRAT*S-C(Sx)-VFAMF-Z (SEQ ID NO: 3149), X-KGTLR-C(Sx)-MS*PEQISSQ-Z (SEQ ID NO: 3150), X-KGTLRYMS*P-C(Sx)-QISSQ-Z (SEQ ID NO: 3151), X-KHKKKKKRPQRADS*D-C(Sx)-FA-Z (SEQ ID NO: 3152), X-KHKKKKKRPQRADS*D-C(Sx)-FS-Z (SEQ ID NO: 3153), X-KHKKKKKRPQRADS*N-C(Sx)-FA-Z (SEQ ID NO: 3154), X-KHKKKKKRPQRADS*N-C(Sx)-FS-Z (SEQ ID NO: 3155), X-KHKKKKKRPQRADSD-C(Sx)-FS*-Z (SEQ ID NO: 3156), X-KHKKKKKRPQRADSN-C(Sx)-FS*-Z (SEQ ID NO: 3157); wherein X— is H or acetyl; and Z is OH or NH2.

In some embodiments, the metal-binding compound of the present invention is selected from the group consisting of: X-KHKKKKKRPQRAES*D-C(Sx)-FA-Z (SEQ ID NO: 3158), X-KHKKKKKRPQRAES*D-C(Sx)-FS-Z (SEQ ID NO: 3159), X-KHKKKKKRPQRAES*N-C(Sx)-FA-Z (SEQ ID NO: 3160), X-KHKKKKKRPQRAES*N-C(Sx)-FS-Z (SEQ ID NO: 3161), X-KHKKKKKRPQRAESD-C(Sx)-FS*-Z (SEQ ID NO: 3162), X-KHKKKKKRPQRAESN-C(Sx)-FS*-Z (SEQ ID NO: 3163), X-KHKKKKKRPQRAHS*D-C(Sx)-FA-Z (SEQ ID NO: 3164), X-KHKKKKKRPQRAHS*D-C(Sx)-FS-Z (SEQ ID NO: 3165), X-KHKKKKKRPQRAHS*N-C(Sx)-FA-Z (SEQ ID NO: 3166), X-KHKKKKKRPQRAHS*N-C(Sx)-FS-Z (SEQ ID NO: 3167), X-KHKKKKKKRPQRAHSD-C(Sx)-FS*-Z (SEQ ID NO: 3168), X-KHKKKKKRPQRAHSN-C(Sx)-FS*-Z (SEQ ID NO: 3169), X-KHKKKKKRPQRATS*D-C(Sx)-FA-Z (SEQ ID NO: 3170), X-KHKKKKKRPQRATS*D-C(Sx)-FS-Z (SEQ ID NO: 3171), X-KHKKKKKRPQRATS*N-C(Sx)-FA-Z (SEQ ID NO: 3172), X-KHKKKKKRPQRATS*N-C(Sx)-FS-Z (SEQ ID NO: 3173), X-KHKKKKKRPQRATSD-C(Sx)-FS*-Z (SEQ ID NO: 3174), X-KHKKKKKRPQRATSN-C(Sx)-FS*-Z (SEQ ID NO: 3175), X-KHKKRKKRPQRADS*D-C(Sx)-FA-Z (SEQ ID NO: 3176), X-KHKKRKKRPQRADS*D-C(Sx)-FS-Z (SEQ ID NO: 3177), X-KHKKRKKRPQRADS*N-C(Sx)-FA-Z (SEQ ID NO: 3178), X-KHKKRKKRPQRADS*N-C(Sx)-FS-Z (SEQ ID NO: 3179), X-KHKKRKKRPQRADSD-C(Sx)-FS*-Z (SEQ ID NO: 3180), X-KHKKRKKRPQRADSN-C(Sx)-FS*-Z (SEQ ID NO: 3181), X-KHKKRKKRPQRAES*D-C(Sx)-FA-Z (SEQ ID NO: 3182), X-KHKKRKKRPQRAES*D-C(Sx)-FS-Z (SEQ ID NO: 3183), X-KHKKRKKRPQRAES*N-C(Sx)-FA-Z (SEQ ID NO: 3184), X-KHKKRKKRPQRAES*N-C(Sx)-FS-Z (SEQ ID NO: 3185), X-KHKKRKKRPQRAESD-C(Sx)-FS*-Z (SEQ ID NO: 3186), X-KHKKRKKRPQRAESN-C(Sx)-FS*-Z (SEQ ID NO: 3187), X-KHKKRKKRPQRAHS*D-C(Sx)-FA-Z (SEQ ID NO: 3188), X-KHKKRKKRPQRAHS*D-C(Sx)-FS-Z (SEQ ID NO: 3189), X-KHKKRKKRPQRAHS*N-C(Sx)-FA-Z (SEQ ID NO: 3190), X-KHKKRKKRPQRAHS*N-C(Sx)-FS-Z (SEQ ID NO: 3191), X-KHKKRKKRPQRAHSD-C(Sx)-FS*-Z (SEQ ID NO: 3192), X-KHKKRKKRPQRAHSN-C(Sx)-FS*-Z (SEQ ID NO: 3193), X-KHKKRKKRPQRATS*D-C(Sx)-FA-Z (SEQ ID NO: 3194), X-KHKKRKKRPQRATS*D-C(Sx)-FS-Z (SEQ ID NO: 3195), X-KHKKRKKRPQRATS*N-C(Sx)-FA-Z (SEQ ID NO: 3196), X-KHKKRKKRPQRATS*N-C(Sx)-FS-Z (SEQ ID NO: 3197), X-KHKKRKKRPQRATSD-C(Sx)-FS*-Z (SEQ ID NO: 3198), X-KHKKRKKRPQRATSN-C(Sx)-FS*-Z (SEQ ID NO: 3199), X-KHKKTKKRPQRADS*D-C(Sx)-FA-Z (SEQ ID NO: 3200), X-KHKKTKKRPQRADS*D-C(Sx)-FS-Z (SEQ ID NO: 3201), X-KHKKTKKRPQRADS*N-C(Sx)-FA-Z (SEQ ID NO: 3202), X-KHKKTKKRPQRADS*N-C(Sx)-FS-Z (SEQ ID NO: 3203), X-KHKKTKKRPQRADSD-C(Sx)-FS*-Z (SEQ ID NO: 3204), X-KHKKTKKRPQRADSN-C(Sx)-FS*-Z (SEQ ID NO: 3205), X-KHKKTKKRPQRAES*D-C(Sx)-FA-Z (SEQ ID NO: 3206), X-KHKKTKKRPQRAES*D-C(Sx)-FS-Z (SEQ ID NO: 3207), X-KHKKTKKRPQRAES*N-C(Sx)-FA-Z (SEQ ID NO: 3208), X-KHKKTKKRPQRAES*N-C(Sx)-FS-Z (SEQ ID NO: 3209), X-KHKKTKKRPQRAESD-C(Sx)-FS*-Z (SEQ ID NO: 3210), X-KHKKTKKRPQRAESN-C(Sx)-FS*-Z (SEQ ID NO: 3211), and X-KHKKTKKRPQRAHS*D-C(Sx)-FA-Z (SEQ ID NO: 3212); wherein X is H or acetyl; and Z is OH or NH2.

In some embodiments, the metal-binding compound of the present invention is selected from the group consisting of: X-KHKKTKKRPQRAHS*D-C(Sx)-FS-Z (SEQ ID NO: 3213), X-KHKKTKKRPQRAHS*N-C(Sx)-FA-Z (SEQ ID NO: 3214), X-KHKKTKKRPQRAHS*N-C(Sx)-FS-Z (SEQ ID NO: 3215), X-KHKKTKKRPQRAHSD-C(Sx)-FS*-Z (SEQ ID NO: 3216), X-KHKKTKKRPQRAHSN-C(Sx)-FS*-Z (SEQ ID NO: 3217), X-KHKKTKKRPQRATS*D-C(Sx)-FA-Z (SEQ ID NO: 3218), X-KHKKTKKRPQRATS*D-C(Sx)-FS-Z (SEQ ID NO: 3219), X-KHKKTKKRPQRATS*N-C(Sx)-FA-Z (SEQ ID NO: 3220), X-KHKKTKKRPQRATS*N-C(Sx)-FS-Z (SEQ ID NO: 3221), X-KHKKTKKRPQRATSD-C(Sx)-FS*-Z (SEQ ID NO: 3222), X-KHKKTKKRPQRATSN-C(Sx)-FS*-Z (SEQ ID NO: 3223), X-KHKKVKKRPQRADS*D-C(Sx)-FA-Z (SEQ ID NO: 3224), X-KHKKVKKRPQRADS*D-C(Sx)-FS-Z (SEQ ID NO: 3225), X-KHKKVKKRPQRADS*N-C(Sx)-FA-Z (SEQ ID NO: 3226), X-KHKKVKKRPQRADS*N-C(Sx)-FS-Z (SEQ ID NO: 3227), X-KHKKVKKRPQRADSD-C(Sx)-FS*-Z (SEQ ID NO: 3228), X-KHKKVKKRPQRADSN-C(Sx)-FS*-Z (SEQ ID NO: 3229), X-KHKKVKKRPQRAES*D-C(Sx)-FA-Z (SEQ ID NO: 3230), X-KHKKVKKRPQRAES*D-C(Sx)-FS-Z (SEQ ID NO: 3231), X-KHKKVKKRPQRAES*N-C(Sx)-FA-Z (SEQ ID NO: 3232), X-KHKKVKKRPQRAES*N-C(Sx)-FS-Z (SEQ ID NO: 3233), X-KHKKVKKRPQRAESD-C(Sx)-FS*-Z (SEQ ID NO: 3234), X-KHKKVKKRPQRAESN-C(Sx)-FS*-Z (SEQ ID NO: 3235), X-KHKKVKKRPQRAHS*D-C(Sx)-FA-Z (SEQ ID NO: 3236), X-KHKKVKKRPQRAHS*D-C(Sx)-FS-Z (SEQ ID NO: 3237), X-KHKKVKKRPQRAHS*N-C(Sx)-FA-Z (SEQ ID NO: 3238), X-KHKKVKKRPQRAHS*N-C(Sx)-FS-Z (SEQ ID NO: 3239), X-KHKKVKKRPQRAHSD-C(Sx)-FS*-Z (SEQ ID NO: 3240), X-KHKKVKKRPQRAHSN-C(Sx)-FS*-Z (SEQ ID NO: 3241), X-KHKKVKKRPQRATS*D-C(Sx)-FA-Z (SEQ ID NO: 3242), X-KHKKVKKRPQRATS*D-C(Sx)-FS-Z (SEQ ID NO: 3243), X-KHKKVKKRPQRATS*N-C(Sx)-FA-Z (SEQ ID NO: 3244), X-KHKKVKKRPQRATS*N-C(Sx)-FS-Z (SEQ ID NO: 3245), X-KHKKVKKRPQRATSD-C(Sx)-FS*-Z (SEQ ID NO: 3246), X-KHKKVKKRPQRATSN-C(Sx)-FS*-Z (SEQ ID NO: 3247), X-KHKTKKKRPQRADS*D-C(Sx)-FA-Z (SEQ ID NO: 3248), X-KHKTKKKRPQRADS*D-C(Sx)-FS-Z (SEQ ID NO: 3249), X-KHKTKKKRPQRADS*N-C(Sx)-FA-Z (SEQ ID NO: 3250), X-KHKTKKKRPQRADS*N-C(Sx)-FS-Z (SEQ ID NO: 3251), X-KHKTKKKRPQRADSD-C(Sx)-FS*-Z (SEQ ID NO: 3252), X-KHKTKKKRPQRADSN-C(Sx)-FS*-Z (SEQ ID NO: 3253), X-KHKTKKKRPQRAES*D-C(Sx)-FA-Z (SEQ ID NO: 3254), X-KHKTKKKRPQRAES*D-C(Sx)-FS-Z (SEQ ID NO: 3255), X-KHKTKKKRPQRAES*N-C(Sx)-FA-Z (SEQ ID NO: 3256), X-KHKTKKKRPQRAES*N-C(Sx)-FS-Z (SEQ ID NO: 3257), X-KHKTKKKRPQRAESD-C(Sx)-FS*-Z (SEQ ID NO: 3258), X-KHKTKKKRPQRAESN-C(Sx)-FS*-Z (SEQ ID NO: 3259), X-KHKTKKKRPQRAHS*D-C(Sx)-FA-Z (SEQ ID NO: 3260), X-KHKTKKKRPQRAHS*D-C(Sx)-FS-Z (SEQ ID NO: 3261), X-KHKTKKKRPQRAHS*N-C(Sx)-FA-Z (SEQ ID NO: 3262), X-KHKTKKKRPQRAHS*N-C(Sx)-FS-Z (SEQ ID NO: 3263), X-KHKTKKKRPQRAHSD-C(Sx)-FS*-Z (SEQ ID NO: 3264), X-KHKTKKKRPQRAHSN-C(Sx)-FS*-Z (SEQ ID NO: 3265), X-KHKTKKKRPQRATS*D-C(Sx)-FA-Z (SEQ ID NO: 3266), X-KHKTKKKRPQRATS*D-C(Sx)-FS-Z (SEQ ID NO: 3267), X-KHKTKKKRPQRATS*N-C(Sx)-FA-Z (SEQ ID NO: 3268), X-KHKTKKKRPQRATS*N-C(Sx)-FS-Z (SEQ ID NO: 3269), X-KHKTKKKRPQRATSD-C(Sx)-FS*-Z (SEQ ID NO: 3270), X-KHKTKKKRPQRATSN-C(Sx)-FS*-Z (SEQ ID NO: 3271), X-KHKTRKKRPQRADS*D-C(Sx)-FA-Z (SEQ ID NO: 3272), X-KHKTRKKRPQRADS*D-C(Sx)-FS-Z (SEQ ID NO: 3273), X-KHKTRKKRPQRADS*N-C(Sx)-FA-Z (SEQ ID NO: 3274), X-KHKTRKKRPQRADS*N-C(Sx)-FS-Z (SEQ ID NO: 3275), X-KHKTRKKRPQRADSD-C(Sx)-FS*-Z (SEQ ID NO: 3276), X-KHKTRKKRPQRADSN-C(Sx)-FS*-Z (SEQ ID NO: 3277), X-KHKTRKKRPQRAES*D-C(Sx)-FA-Z (SEQ ID NO: 3278), X-KHKTRKKRPQRAES*D-C(Sx)-FS-Z (SEQ ID NO: 3279), and X-KHKTRKKRPQRAES*N-C(Sx)-FA-Z (SEQ ID NO: 3280); wherein X is H or acetyl; and Z is OH or NH2.

In some embodiments, the metal-binding compound of the present invention is selected from the group consisting of: X-KHKTRKKRPQRAES*N-C(Sx)-FS-Z (SEQ ID NO: 3281), X-KHKTRKKRPQRAESD-C(Sx)-FS*-Z (SEQ ID NO: 3282), X-KHKTRKKRPQRAESN-C(Sx)-FS*-Z (SEQ ID NO: 3283), X-KHKTRKKRPQRAHS*D-C(Sx)-FA-Z (SEQ ID NO: 3284), X-KHKTRKKRPQRAHS*D-C(Sx)-FS-Z (SEQ ID NO: 3285), X-KHKTRKKRPQRAHS*N-C(Sx)-FA-Z (SEQ ID NO: 3286), X-KHKTRKKRPQRAHS*N-C(Sx)-FS-Z (SEQ ID NO: 3287), X-KHKTRKKRPQRAHSD-C(Sx)-FS*-Z (SEQ ID NO: 3288), X-KHKTRKKRPQRAHSN-C(Sx)-FS*-Z (SEQ ID NO: 3289), X-KHKTRKKRPQRATS*D-C(Sx)-FA-Z (SEQ ID NO: 3290), X-KHKTRKKRPQRATS*D-C(Sx)-FS-Z (SEQ ID NO: 3291), X-KHKTRKKRPQRATS*N-C(Sx)-FA-Z (SEQ ID NO: 3292), X-KHKTRKKRPQRATS*N-C(Sx)-FS-Z (SEQ ID NO: 3293), X-KHKTRKKRPQRATSD-C(Sx)-FS*-Z (SEQ ID NO: 3294), X-KHKTRKKRPQRATSN-C(Sx)-FS*-Z (SEQ ID NO: 3295), X-KHKTIKKRPQRADS*D-C(Sx)-FA-Z (SEQ ID NO: 3296), X-KHKITKKRPQRADS*D-C(Sx)-FS-Z (SEQ ID NO: 3297), X-KHKTTKKRPQRADS*N-C(Sx)-FA-Z (SEQ ID NO: 3298), X-KHKITKKRPQRADS*N-C(Sx)-FS-Z (SEQ ID NO: 3299), X-KHKITKKRPQRADSD-C(Sx)-FS*-Z (SEQ ID NO: 3300), X-KHKTTKKRPQRADSN-C(Sx)-FS*-Z (SEQ ID NO: 3301), X-KHKTTKKRPQRAES*D-C(Sx)-FA-Z (SEQ ID NO: 3302), X-KHKTITKKRPQRAES*D-C(Sx)-FS-Z (SEQ ID NO: 3303), X-KHKITKKRPQRAES*N-C(Sx)-FA-Z (SEQ ID NO: 3304), X-KHKITKKRPQRAES*N-C(Sx)-FS-Z (SEQ ID NO: 3305), X-KHKTTKKRPQRAESD-C(Sx)-FS*-Z (SEQ ID NO: 3306), X-KHKITKKRPQRAESN-C(Sx)-FS*-Z (SEQ ID NO: 3307), X-KHKTIKKRPQRAHS*D-C(Sx)-FA-Z (SEQ ID NO: 3308), X-KHKITKKRPQRAHS*D-C(Sx)-FS-Z (SEQ ID NO: 3309), X-KHKTTKKRPQRAHS*N-C(Sx)-FA-Z (SEQ ID NO: 3310), X-KHKTKKRPQRAHS*N-C(Sx)-FS-Z (SEQ ID NO: 3311), X-KHKTTKKRPQRAHSD-C(Sx)-FS*-Z (SEQ ID NO: 3312), X-KHKTTKKRPQRAHSN-C(Sx)-FS*-Z (SEQ ID NO: 3313), X-KHKTTKKRPQRATS*D-C(Sx)-FA-Z (SEQ ID NO: 3314), X-KHKTTKKRPQRATS*D-C(Sx)-FS-Z (SEQ ID NO: 3315), X-KHKTTKKRPQRATS*N-C(Sx)-FA-Z (SEQ ID NO: 3316), X-KHKTVKKRPQRATS*N-C(Sx)-FS-Z (SEQ ID NO: 3317), X-KHKTTKKRPQRATSD-C(Sx)-FS*-Z (SEQ ID NO: 3318), X-KHKTITKKRPQRATSN-C(Sx)-FS*-Z (SEQ ID NO: 3319), X-KHKTVKKRPQRADS*D-C(Sx)-FA-Z (SEQ ID NO: 3320), X-KHKTVKKRPQRADS*D-C(Sx)-FS-Z (SEQ ID NO: 3321), X-KHKTVKKRPQRADS*N-C(Sx)-FA-Z (SEQ ID NO: 3322), X-KHKTVKKRPQRADS*N-C(Sx)-FS-Z (SEQ ID NO: 3323), and X-KHKTVKKRPQRADSD-C(Sx)-FS*-Z (SEQ ID NO: 3324); wherein X is H or acetyl; and Z is OH or NH2.

In some embodiments, the metal-binding compound of the present invention is selected from the group consisting of: X-KHKTVKKRPQRADSN-C(Sx)-FS*-Z (SEQ ID NO: 3325), X-KHKTVKKRPQRAES*D-C(Sx)-FA-Z (SEQ ID NO: 3326), X-KHKTVKKRPQRAES*D-C(Sx)-FS-Z (SEQ ID NO: 3327), X-KHKTVKKRPQRAES*N-C(Sx)-FA-Z (SEQ ID NO: 3328), X-KHKTVKKRPQRAES*N-C(Sx)-FS-Z (SEQ ID NO: 3329), X-KHKTVKKRPQRAESD-C(Sx)-FS*-Z (SEQ ID NO: 3330), X-KHKTVKKRPQRAESN-C(Sx)-FS*-Z (SEQ ID NO: 3331), X-KHKTVKKRPQRAHS*D-C(Sx)-FA-Z (SEQ ID NO: 3332), X-KHKTVKKRPQRAHS*D-C(Sx)-FS-Z (SEQ ID NO: 3333), X-KHKTVKKRPQRAHS*N-C(Sx)-FA-Z (SEQ ID NO: 3334), X-KHKTVKKRPQRAHS*N-C(Sx)-FS-Z (SEQ ID NO: 3335), X-KHKTVKKRPQRAHSD-C(Sx)-FS*-Z (SEQ ID NO: 3336), X-KHKTVKKRPQRAHSN-C(Sx)-FS*-Z (SEQ ID NO: 3337), X-KHKTVKKRPQRATS*D-C(Sx)-FA-Z (SEQ ID NO: 3338), X-KHKTVKKRPQRATS*D-C(Sx)-FS-Z (SEQ ID NO: 3339), X-KHKTVKKRPQRATS*N-C(Sx)-FA-Z (SEQ ID NO: 3340), X-KHKTVKKRPQRATS*N-C(Sx)-FS-Z (SEQ ID NO: 3341), X-KHKTVKKRPQRATSD-C(Sx)-FS*-Z (SEQ ID NO: 3342), X-KHKTVKKRPQRATSN-C(Sx)-FS*-Z (SEQ ID NO: 3343), X-KHRPQRAT*S-C(Sx)-VFAMF-Z (SEQ ID NO: 3344), X-KIKTTKKRPQRATS*N-C(Sx)-FS-Z (SEQ ID NO: 3345), X-KIKTTKKRPQRATSN-C(Sx)-FS*-Z (SEQ ID NO: 3346), X-KIRPQRAT*S-C(Sx)-VFAMF-Z (SEQ ID NO: 3347), X-KK[Nle]PQRAT*S-C(Sx)-VFAMF-Z (SEQ ID NO: 3348), X-KK-C(Sx)-AS*FKK-Z (SEQ ID NO: 3349), X-KK-C(Sx)-AS*FRR-Z (SEQ ID NO: 3350), X-KKCDLGYAKDVDQGS*L-C(Sx)-TSFVGTLQYLAPECKK-Z (SEQ ID NO: 1017), X-KKCDLGYAKDVDQGSL-C(Sx)-TS*FVGTLQYLAPECKK-Z (SEQ ID NO: 1018), X-KKCVSGQLIDS*M-C(Sx)-NSFVGTRSYCKK-Z (SEQ ID NO: 1007), X-KKCVSGQLIDSM-C(Sx)-NS*FVGTRSYCKK-Z (SEQ ID NO: 1008), X-KKDPQRAT*S-C(Sx)-VFAMF-Z (SEQ ID NO: 3351), X-KKEPQRAT*S-C(Sx)-VFAMF-Z (SEQ ID NO: 3352), X-KKERLLDDRHDS*G-C(Sx)-DSMKDEE-Z (SEQ ID NO: 3353), X-KKERLLDDRHDSG-C(Sx)-DS*MKDEE-Z (SEQ ID NO: 3354), X-KKERLLDDRHDSGLDS*M-C(Sx)-DEE-Z (SEQ ID NO: 3355), X-KKFPQRAT*S-C(Sx)-VFAMF-Z (SEQ ID NO: 3356), X-KKGPQRAT*S-C(Sx)-VFAMF-Z (SEQ ID NO: 3357), X-KKHPQRAT*S-C(Sx)-VFAMF-Z (SEQ ID NO: 3358), and X-KKIPQRAT*S-C(Sx)-VFAMF-Z (SEQ ID NO: 3359); wherein X— is H or acetyl; and Z is OH or NH2.

In some embodiments, the metal-binding compound of the present invention is selected from the group consisting of: X-KKIS*G-C(Sx)-LSPI[Nle]TEQ-Z (SEQ ID NO: 3360), X-KKISG-C(Sx)-LS*PI[Nle]TEQ-Z (SEQ ID NO: 3361), X-KKISGRLS*P-C(Sx)-[Nle]TEQ-Z (SEQ ID NO: 3362), X-KKISGRLSP-C(Sx)-[Nle]T*EQ-Z (SEQ ID NO: 3363), X-KKKADKQFLIS*P-C(Sx)-ASP-Z (SEQ ID NO: 3364), X-KKKADKQFLISP-C(Sx)-AS*P-Z (SEQ ID NO: 3365), X-KKKAEPLPPSYV-C(Sx)-AS*-Z (SEQ ID NO: 3366), X-KKKAGTSF[Nle][Nle]T*P-C(Sx)-VVTR-Z (SEQ ID NO: 3367), X-KKKANPSPPPS*P-C(Sx)-QQINL-Z (SEQ ID NO: 3368), X-KKKAPLSTWSGS*P-C(Sx)-YAAPE-Z (SEQ ID NO: 3369), X-KKKDGR[Nle]VQLS*P-C(Sx)-ALAAP-Z (SEQ ID NO: 3370), X-KKKFQGQLLGT*I-C(Sx)-F[Nle]APE-Z (SEQ ID NO: 3371), X-KKKGDNVLINT*Y-C(Sx)-GVLKI-Z (SEQ ID NO: 3372), X-KKKGHITITPT*P-C(Sx)-QFL-Z (SEQ ID NO: 3373), X-KKKGINPSTET*F-C(Sx)-GTLQY-Z (SEQ ID NO: 3374), X-KKKGINPSTETF-C(Sx)-GT*LQY-Z (SEQ ID NO: 3375), X-KKKGNLLPMS*P-C(Sx)-EFD-Z (SEQ ID NO: 3376), X-KKKGQSSAAAT*P-C(Sx)-TTGTK-Z (SEQ ID NO: 3377), X-KKKGTQREGVSS*L-C(Sx)-SSSL-Z (SEQ ID NO: 3378), X-KKKGTQREGVSSL-C(Sx)-SS*SL-Z (SEQ ID NO: 3379), X-KKKGVITITPT*P-C(Sx)-GQYFY-Z (SEQ ID NO: 3380), X-KKKHQLFRGFS*F-C(Sx)-AITSD-Z (SEQ ID NO: 3381), X-KKKIHFWSSLS*P-C(Sx)-APLSP-Z (SEQ ID NO: 3382), X-KKKKKKKRPQRADS*D-C(Sx)-FA-Z (SEQ ID NO: 3383), X-KKKKKKKRPQRADS*D-C(Sx)-FS-Z (SEQ ID NO: 3384), X-KKKKKKKRPQRADS*N-C(Sx)-FA-Z (SEQ ID NO: 3385), X-KKKKKKKRPQRADS*N-C(Sx)-FS-Z (SEQ ID NO: 3386), X-KKKKKKKRPQRADSD-C(Sx)-FS*-Z (SEQ ID NO: 3387), X-KKKKKKKRPQRADSN-C(Sx)-FS*-Z (SEQ ID NO: 3388), X-KKKKKKKKKKRPQRAES*D-C(Sx)-FA-Z (SEQ ID NO: 3389), X-KKKKKKKRPQRAES*D-C(Sx)-FS-Z (SEQ ID NO: 3390), X-KCKKKKKKRPQRAHS*N-C(Sx)-FA-Z (SEQ ID NO: 3391), X-KCKKKKKKRPQRAES*N-C(Sx)-FS-Z (SEQ ID NO: 3392), X-KCKKKKKKRPQRAESD-C(Sx)-FS*-Z (SEQ ID NO: 3393), X-KCKKKKKKRPQRAESN-C(Sx)-FS*-Z (SEQ ID NO: 3394), X-KKKKKKKRPQRAHS*D-C(Sx)-FA-Z (SEQ ID NO: 3395), X-KCKKKKKKRPQRAHS*D-C(Sx)-FS-Z (SEQ ID NO: 3396), X-KCKKKKKKRPQRAHS*N-C(Sx) FA-Z (SEQ ID NO: 3397), X-KCKKKKKKRPQRAHS*N-C(Sx)-FS-Z (SEQ ID NO: 3398), X-KCKKKKKKRPQRAHSD-C(Sx)-FS*-Z (SEQ ID NO: 3399), X-KKKKKKKRPQRAHSN-C(Sx)-FS*-Z (SEQ ID NO: 3400), X-KKKKKKKRPQRATS*D-C(Sx)-FA-Z (SEQ ID NO: 3401), X-KCKKKKKKRPQRATS*D-C(Sx)-FS-Z (SEQ ID NO: 3402), X-KCKKKKKKRPQRATS*N-C(Sx)-FA-Z (SEQ ID NO: 3403), X-KCKKKKKKRPQRATS*N-C(Sx)-FS-Z (SEQ ID NO: 3404), X-KCKKKKKKRPQRATSD-C(Sx)-FS*-Z (SEQ ID NO: 3405), X-KCKKKKKKRPQRATSN-C(Sx)-FS*-Z (SEQ ID NO: 3406), X-KCKKKRKKRPQRADS*D-C(Sx)-FA-Z (SEQ ID NO: 3407), X-KCKKKRKKRPQRADS*D-C(Sx)-FS-Z (SEQ ID NO: 3408), X-KKKKRKKRPQRADS*N-C(Sx)-FA-Z (SEQ ID NO: 3409), X-KCKKKRKKRPQRADS*N-C(Sx)-FS-Z (SEQ ID NO: 3410), X-KCKKKRKKRPQRADSD-C(Sx)-FS*-Z (SEQ ID NO: 3411), X-KKKKRKKRPQRADSN-C(Sx)-FS*-Z (SEQ ID NO: 3412), X-KCKKKRKKRPQRAES*D-C(Sx)-FA-Z (SEQ ID NO: 3413), X-KCKKKRKKRPQRAES*D-C(Sx)-FS-Z (SEQ ID NO: 3414), X-KCKKKRKKRPQRAES*N-C(Sx)-FA-Z (SEQ ID NO: 3415), X-KCKKKRKKRPQRAES*N-C(Sx)-FS-Z (SEQ ID NO: 3416), X-KCKKKRKKRPQRAESD-C(Sx)-FS*-Z (SEQ ID NO: 3417), X-KCKKKRKKRPQRAESN-C(Sx)-FS*-Z (SEQ ID NO: 3418), X-KCKKKRKKRPQRAHS*D-C(Sx)-FA-Z (SEQ ID NO: 3419), X-KCKKKKKKRPQRAHS*D-C(Sx)-FS-Z (SEQ ID NO: 3420), X-KKKKRKKRPQRAHS*N-C(Sx)-FA-Z (SEQ ID NO: 3421), X-KCKKKRKKRPQRAHS*N-C(Sx)-FS-Z (SEQ ID NO: 3422), X-KCKKKRKKRPQRAHSD-C(Sx)-FS*-Z (SEQ ID NO: 3423), X-KKKKRKKRPQRAHSN-C(Sx)-FS*-Z (SEQ ID NO: 3424), X-KKKKRKKRPQRATS*D-C(Sx)-FA-Z (SEQ ID NO: 3425), X-KCKKKRKKRPQRATS*D-C(Sx)-FS-Z (SEQ ID NO: 3426), X-KCKKKRKKRPQRATS*N-C(Sx)-FA-Z (SEQ ID NO: 3427), and X-KCKKKRKKRPQRATS*N-C(Sx)-FS-Z (SEQ ID NO: 3428); wherein X is H or acetyl; and Z is OH or NH2.

In some embodiments, the metal-binding compound of the present invention is selected from the group consisting of: X-KCKKKRKKRPQRATSD-C(Sx)-FS*-Z (SEQ ID NO: 3429), X-KKKKRKKRPQRATSN-C(Sx)-FS*-Z (SEQ ID NO: 3430), X-KKKKTKKRPQRADS*D-C(Sx)-FA-Z (SEQ ID NO: 3431), X-KKKKTKKRPQRADS*D-C(Sx)-FS-Z (SEQ ID NO: 3432), X-KKKKTKKRPQRADS*N-C(Sx)-FA-Z (SEQ ID NO: 3433), X-KKKKTKKRPQRADS*N-C(Sx)-FS-Z (SEQ ID NO: 3434), X-KKKKTKKRPQRADSD-C(Sx)-FS*-Z (SEQ ID NO: 3435), X-KKKKTKKRPQRADSN-C(Sx)-FS*-Z (SEQ ID NO: 3436), X-KKKKTKKRPQRAES*D-C(Sx)-FA-Z (SEQ ID NO: 3437), X-KKKKTKKRPQRAES*D-C(Sx)-FS-Z (SEQ ID NO: 3438), X-KKKKTKKRPQRAES*N-C(Sx)-FA-Z (SEQ ID NO: 3439), X-KKKKTKKRPQRAES*N-C(Sx)-FS-Z (SEQ ID NO: 3440), X-KKKKTKKRPQRAESD-C(Sx)-FS*-Z (SEQ ID NO: 3441), X-KKKKTKKRPQRAESN-C(Sx)-FS*-Z (SEQ ID NO: 3442), X-KKKKTKKRPQRAHS*D-C(Sx)-FA-Z (SEQ ID NO: 3443), X-KKKKTKKRPQRAHS*D-C(Sx)-FS-Z (SEQ ID NO: 3444), X-KKKKTKKRPQRAHS*N-C(Sx)-FA-Z (SEQ ID NO: 3445), X-KKKKTKKRPQRAHS*N-C(Sx)-FS-Z (SEQ ID NO: 3446), X-KKKKTKKRPQRAHSD-C(Sx)-FS*-Z (SEQ ID NO: 3447), X-KKKKTKKRPQRAHSN-C(Sx)-FS*-Z (SEQ ID NO: 3448), X-KKKKTKKRPQRATS*D-C(Sx)-FA-Z (SEQ ID NO: 3449), X-KKKKTKKRPQRATS*D-C(Sx)-FS-Z (SEQ ID NO: 3450), X-KKKKTKKRPQRATS*N-C(Sx)-FA-Z (SEQ ID NO: 3451), X-KKKKTKKRPQRATS*N-C(Sx)-FS-Z (SEQ ID NO: 3452), X-KKKKTKKRPQRATSD-C(Sx)-FS*-Z (SEQ ID NO: 3453), X-KKKKTKKRPQRATSN-C(Sx)-FS*-Z (SEQ ID NO: 3454), X-KKKKVKKRPQRA[hE]S*D-C(Sx)-FS-Z (SEQ ID NO: 3455), X-KKKKVKKRPQRA[hE]SD-C(Sx)-FS*-Z (SEQ ID NO: 3456), X-KKKKVKKRPQRADS*D-C(Sx)-FA-Z (SEQ ID NO: 3457), X-KKKKVKKRPQRADS*D-C(Sx)-FS-Z (SEQ ID NO: 3458), X-KKKKVKKRPQRADS*N-C(Sx)-FA-Z (SEQ ID NO: 3459), X-KKKKVKKRPQRADS*N-C(Sx)-FS-Z (SEQ ID NO: 3460), X-KKKKVKKRPQRADSD-C(Sx)-FS*-Z (SEQ ID NO: 3461), and X-KKKKVKKRPQRADSN-C(Sx)-FS*-Z (SEQ ID NO: 3462); wherein X is H or acetyl; and Z is OH or NH2.

In some embodiments, the metal-binding compound of the present invention is selected from the group consisting of: X-KKKKVKKRPQRAES*D-C(Sx)-FA-Z (SEQ ID NO: 3463), X-KKKKVKKRPQRAES*D-C(Sx)-FS-Z (SEQ ID NO: 3464), X-KKKKVKKRPQRAES*N-C(Sx)-FA-Z (SEQ ID NO: 3465), X-KKKKVKKRPQRAES*N-C(Sx)-FS-Z (SEQ ID NO: 3466), X-KKKKVKKRPQRAESD-C(Sx)-FS*-Z (SEQ ID NO: 3467), X-KKKKVKKRPQRAESN-C(Sx)-FS*-Z (SEQ ID NO: 3468), X-KKKKVKKRPQRAHS*D-C(Sx)-FA-Z (SEQ ID NO: 3469), X-KKKKVKKRPQRAHS*D-C(Sx)-FS-Z (SEQ ID NO: 3470), X-KKKKVKKRPQRAHS*N-C(Sx)-FA-Z (SEQ ID NO: 3471), X-KKKKVKKRPQRAHS*N-C(Sx)-FS-Z (SEQ ID NO: 3472), X-KKKKVKKRPQRAHSD-C(Sx)-FS*-Z (SEQ ID NO: 3473), X-KKKKVKKRPQRAHSN-C(Sx)-FS*-Z (SEQ ID NO: 3474), X-KKKKVKKRPQRATS*D-C(Sx)-FA-Z (SEQ ID NO: 3475), X-KKKKVKKRPQRATS*D-C(Sx)-FS-Z (SEQ ID NO: 3476), X-KKKKVKKRPQRATS*N-C(Sx)-FA-Z (SEQ ID NO: 3477), X-KKKKVKKRPQRATS*N-C(Sx)-FS-Z (SEQ ID NO: 3478), X-KKKKVKKRPQRATSD-C(Sx)-FS*-Z (SEQ ID NO: 3479), X-KKKKVKKRPQRATSN-C(Sx)-FS*-Z (SEQ ID NO: 3480), X-KKKLAPNPSFS*P-C(Sx)-PGFTP-Z (SEQ ID NO: 1102), X-KKKLIDS[Nle]ANS*F-C(Sx)-GTRSY-Z (SEQ ID NO: 3481), X-KKKLIDS[Nle]ANSF-C(Sx)-GT*RSY-Z (SEQ ID NO: 3482), X-KKKLPPVFSGT*P-C(Sx)-GSGAG-Z (SEQ ID NO: 3483), X-KKKLPPVFSGTP-C(Sx)-GS*GAG-Z (SEQ ID NO: 3484), X-KKKLSNSQGQS*P-C(Sx)-VPFPA-Z (SEQ ID NO: 3485), X-KKKLTDPRLLS*P-C(Sx)-QPALQ-Z (SEQ ID NO: 3486), X-KKKNVH[Nle]VSTT*L-C(Sx)-VDSR-Z (SEQ ID NO: 3487), X-KKKPLSPFPVS*P-C(Sx)-AGPGS-Z (SEQ ID NO: 3488), X-KKKPQPLRS*P-C(Sx)-LDNPT-Z (SEQ ID NO: 3489), X-KKKPQRAT*S-C(Sx)-VFAMF-Z (SEQ ID NO: 3490), X-KKKPSAPA-C(Sx)-ST*PGIRDSA-Z (SEQ ID NO: 3491), X-KKKPSAPALST*P-C(Sx)-IRDSA-Z (SEQ ID NO: 3492), X-KKKPSAPALST*PG-C(Sx)-RDSA-Z (SEQ ID NO: 3493), X-KKKQ[Nle]AGTPIT*P-C(Sx)-KDGFT-Z (SEQ ID NO: 3494), X-KKKQAATGFGS*P-C(Sx)-QYSAD-Z (SEQ ID NO: 3495), X-KKKQGPAPVGT*P-C(Sx)-FNRQH-Z (SEQ ID NO: 3496), X-KKKQLSKWPGS*P-C(Sx)-SRSSD-Z (SEQ ID NO: 3497), X-KKKQNRSGAMS*P-C(Sx)-SWNSD-Z (SEQ ID NO: 3498), X-KKKQS[Nle]VVPQT*P-C(Sx)-HTARV-Z (SEQ ID NO: 3499), X-KKKQS[Nle]VVPQTP-C(Sx)-HT*ARV-Z (SEQ ID NO: 3500), X-KKKQSAQS-C(Sx)-AT*PVVSVAT-Z (SEQ ID NO: 3501), X-KKKQSAQSLAT*P-C(Sx)-VSVAT-Z (SEQ ID NO: 3502), X-KKKQSAQSLATP-C(Sx)-VS*VAT-Z (SEQ ID NO: 3503), X-KKKRPASVNGS*P-C(Sx)-ATSES-Z (SEQ ID NO: 3504), X-KKKRPASVNGSP-C(Sx)-AT*SES-Z (SEQ ID NO: 3505), X-KKKS[Nle]DGLFGT*I-C(Sx)-YLP-Z (SEQ ID NO: 3506), X-KKKSALQG-C(Sx)-NS*PG[Nle]LSLG-Z (SEQ ID NO: 3507), X-KKKSALQGFNS*P-C(Sx)-[Nle]LSLG-Z (SEQ ID NO: 3508), X-KKKSASGSAFS*P-C(Sx)-PGSAA-Z (SEQ ID NO: 3509), X-KKKSAVNGTSS*A-C(Sx)-TNLEA-Z (SEQ ID NO: 3510), and X-KKKSGYS*S-C(Sx)-GSPGTPGSR-Z (SEQ ID NO: 3511); wherein X— is H or acetyl; and Z is OH or NH2.

In some embodiments, the metal-binding compound of the present invention is selected from the group consisting of: X-KKKSGYSS-C(Sx)-GS*PGTPGSR-Z (SEQ ID NO: 3512), X-KKKSGYSSPGS*P-C(Sx)-TPGSR-Z (SEQ ID NO: 3513), X-KKKSGYSSPGS*PG-C(Sx)-PGSR-Z (SEQ ID NO: 3514), X-KKKSNGHITIT*P-C(Sx)-PTQFL-Z (SEQ ID NO: 3515), X-KKKSNGHITITP-C(Sx)-PT*QFL-Z (SEQ ID NO: 3516), X-KKKSNGLVTIT*P-C(Sx)-SSQFL-Z (SEQ ID NO: 1120), X-KKKSNGLVTITP-C(Sx)-SS*QFL-Z (SEQ ID NO: 3517), X-KKKSNGVITIT*P-C(Sx)-PPGQY-Z (SEQ ID NO: 3518), X-KKKSNPALLSS*P-C(Sx)-YYSAA-Z (SEQ ID NO: 3519), X-KKKSPLNITST*P-C(Sx)-PDASS-Z (SEQ ID NO: 3520), X-KKKSVPSAAVT*P-C(Sx)-NESLQ-Z (SEQ ID NO: 3521), X-KKKSVSAATLT*P-C(Sx)-SQAVT-Z (SEQ ID NO: 3522), X-KKKTETFTGT*L-C(Sx)-Y[Nle]APE-Z (SEQ ID NO: 3523), X-KKKTKKKRPQRADS*D-C(Sx)-FA-Z (SEQ ID NO: 3524), X-KKKTKKKRPQRADS*D-C(Sx)-FS-Z (SEQ ID NO: 3525), X-KKKTKKKRPQRADS*N-C(Sx)-FA-Z (SEQ ID NO: 3526), X-K KTKKKRPQRADS*N-C(Sx)-FS-Z (SEQ ID NO: 3527), X-KKKTKKKRPQRADSD-C(Sx)-FS*-Z (SEQ ID NO: 3528), X-KKKTKKKRPQRADSN-C(Sx)-FS*-Z (SEQ ID NO: 3529), X-KKKTKKKRPQRAES*D-C(Sx)-FA-Z (SEQ ID NO: 3530), X-KTKTKKKRPQRAES*D-C(Sx)-FS-Z (SEQ ID NO: 3531), X-KKKTKKKRPQRAES*N-C(Sx)-FA-Z (SEQ ID NO: 3532), X-KKKTKKKRPQRAES*N-C(Sx)-FS-Z (SEQ ID NO: 3533), X-KKKTKKKRPQRAESD-C(Sx)-FS*-Z (SEQ ID NO: 3534), X-KTKTKKKRPQRAESN-C(Sx)-FS*-Z (SEQ ID NO: 3535), X-KKKTKKKRPQRAHS*D-C(Sx)-FA-Z (SEQ ID NO: 3536), X-KKKTKKKRPQRAHS*D-C(Sx)-FS-Z (SEQ ID NO: 3537), X-KKKTKKKRPQRAHS*N-C(Sx)-FA-Z (SEQ ID NO: 3538), X-K KTKKKRPQRAHS*N-C(Sx)-FS-Z (SEQ ID NO: 3539), X-KKKTKKKRPQRAHSD-C(Sx)-FS*-Z (SEQ ID NO: 3540), X-KKKTKKKRPQRAHSN-C(Sx)-FS*-Z (SEQ ID NO: 3541), X-KKKTKKKRPQRATS*D-C(Sx)-FA-Z (SEQ ID NO: 3542), X-KKKTKKKRPQRATS*D-C(Sx)-FS-Z (SEQ ID NO: 3543), X-KKKTKKKRPQRATS*N-C(Sx)-FA-Z (SEQ ID NO: 3544), X-KKKTKKKRPQRATS*N-C(Sx)-FS-Z (SEQ ID NO: 3545), X-KKKTKKKRPQRATSD-C(Sx)-FS*-Z (SEQ ID NO: 3546), X-KKKTKKKRPQRATSN-C(Sx)-FS*-Z (SEQ ID NO: 3547), X-KKKTPVVSVAT*P-C(Sx)-LPGQG-Z (SEQ ID NO: 3548), X-KKKTRKKRPQRADS*D-C(Sx)-FA-Z (SEQ ID NO: 3549), X-KKKTRKKRPQRADS*D-C(Sx)-FS-Z (SEQ ID NO: 3550), X-KKKTRKKRPQRADS*N-C(Sx)-FA-Z (SEQ ID NO: 3551), X-KKKTRKKRPQRADS*N-C(Sx)-FS-Z (SEQ ID NO: 3552), X-KKKTRKKRPQRADSD-C(Sx)-FS*-Z (SEQ ID NO: 3553), X-KKKTRKKRPQRADSN-C(Sx)-FS*-Z (SEQ ID NO: 3554), X-KKKTRKKRPQRAES*D-C(Sx)-FA-Z (SEQ ID NO: 3555), X-KKKTRKKRPQRAES*D-C(Sx)-FS-Z (SEQ ID NO: 3556), X-KKKTRKKRPQRAES*N-C(Sx)-FA-Z (SEQ ID NO: 3557), X-KKKTRKKRPQRAES*N-C(Sx)-FS-Z (SEQ ID NO: 3558), X-KKKTRKKRPQRAESD-C(Sx)-FS*-Z (SEQ ID NO: 3559), X-KKKTRKKRPQRAESN-C(Sx)-FS*-Z (SEQ ID NO: 3560), X-KKKTRKKRPQRAHS*D-C(Sx)-FA-Z (SEQ ID NO: 3561), X-KKKTRKKRPQRAHS*D-C(Sx)-FS-Z (SEQ ID NO: 3562), X-KKKTRKKRPQRAHS*N-C(Sx)-FA-Z (SEQ ID NO: 3563), X-KKKTRKKRPQRAHS*N-C(Sx)-FS-Z (SEQ ID NO: 3564), X-KKKTRKKRPQRAHSD-C(Sx)-FS*-Z (SEQ ID NO: 3565), X-KKKTRKKRPQRAHSN-C(Sx)-FS*-Z (SEQ ID NO: 3566), and X-KKKTRKKRPQRATS*D-C(Sx)-FA-Z (SEQ ID NO: 3567); wherein X is H or acetyl; and Z is OH or NH2.

In some embodiments, the metal-binding compound of the present invention is selected from the group consisting of: X-KKKTRKKRPQRATS*D-C(Sx)-FS-Z (SEQ ID NO: 3568), X-KKKTRKKRPQRATS*N-C(Sx)-FA-Z (SEQ ID NO: 3569), X-KKKTRKKRPQRATS*N-C(Sx)-FS-Z (SEQ ID NO: 3570), X-KKKTRKKRPQRATSD-C(Sx)-FS*-Z (SEQ ID NO: 3571), X-KKKTRKKRPQRATSN-C(Sx)-FS*-Z (SEQ ID NO: 3572), X-KKKTTGTKSNT*P-C(Sx)-SSVPS-Z (SEQ ID NO: 3573), X-KKKTTGTKSNTP-C(Sx)-SS*VPS-Z (SEQ ID NO: 3574), X-KKKTKKRPQRADS*D-C(Sx)-FA-Z (SEQ ID NO: 3575), X-KKKTTKKRPQRADS*D-C(Sx)-FS-Z (SEQ ID NO: 3576), X-KKKTTKKRPQRADS*N-C(Sx)-FA-Z (SEQ ID NO: 3577), X-KKKTTKKRPQRADS*N-C(Sx)-FS-Z (SEQ ID NO: 3578), X-KKKTTKKRPQRADSD-C(Sx)-FS*-Z (SEQ ID NO: 3579), X-KKKTTKKRPQRADSN-C(Sx)-FS*-Z (SEQ ID NO: 3580), X-KKKTKKRPQRAES*D-C(Sx)-FA-Z (SEQ ID NO: 3581), X-KKKITKKRPQRAES*D-C(Sx)-FS-Z (SEQ ID NO: 3582), X-KKKTKKRPQRAES*N-C(Sx)-FA-Z (SEQ ID NO: 3583), X-KKKTTKKRPQRAES*N-C(Sx)-FS-Z (SEQ ID NO: 3584), X-KKKTTKKRPQRAESD-C(Sx)-FS*-Z (SEQ ID NO: 3585), X-KKKTITKKRPQRAESN-C(Sx)-FS*-Z (SEQ ID NO: 3586), X-KKKTTKKRPQRAHS*D-C(Sx)-FA-Z (SEQ ID NO: 3587), X-KKKTTKKRPQRAHS*D-C(Sx)-FS-Z (SEQ ID NO: 3588), X-KKKTTKKRPQRAHS*N-C(Sx)-FA-Z (SEQ ID NO: 3589), X-KKKTTKKRPQRAHS*N-C(Sx)-FS-Z (SEQ ID NO: 3590), X-KKKTKKRPQRAHSD-C(Sx)-FS*-Z (SEQ ID NO: 3591), X-KKKTTKKRPQRAHSN-C(Sx)-FS*-Z (SEQ ID NO: 3592), X-KKKTTKKRPQRATS*D-C(Sx)-FA-Z (SEQ ID NO: 3593), X-KKKITKKRPQRATS*D-C(Sx)-FS-Z (SEQ ID NO: 3594), X-KKKTKKRPQRATS*N-C(Sx)-FA-Z (SEQ ID NO: 3595), X-KKKTKKRPQRATS*N-C(Sx)-FS-Z (SEQ ID NO: 3596), X-KKKTTKKRPQRATSD-C(Sx)-FS*-Z (SEQ ID NO: 3597), X-KKKTKKRPQRATSN-C(Sx)-FS*-Z (SEQ ID NO: 3598), X-KKKTQNALQT*P-C(Sx)-YTPYY-Z (SEQ ID NO: 3599), X-KKKTTQNALQTP-C(Sx)-YT*PYY-Z (SEQ ID NO: 3600), X-KKKTTSSTPSS*P-C(Sx)-PFPTS-Z (SEQ ID NO: 3601), X-KKKTVKKRPQRADS*D-C(Sx)-FA-Z (SEQ ID NO: 3602), X-KKKTVKKRPQRADS*D-C(Sx)-FS-Z (SEQ ID NO: 3603), X-KKKTVKKRPQRADS*N-C(Sx)-FA-Z (SEQ ID NO: 3604), X-KKKTVKKRPQRADS*N-C(Sx)-FS-Z (SEQ ID NO: 3605), X-KKKTVKKRPQRADSD-C(Sx)-FS*-Z (SEQ ID NO: 3606), X-KKKTVKKRPQRADSN-C(Sx)-FS*-Z (SEQ ID NO: 3607), X-KKKTVKKRPQRAES*D-C(Sx)-FA-Z (SEQ ID NO: 3608), X-KKKTVKKRPQRAES*D-C(Sx)-FS-Z (SEQ ID NO: 3609), X-KKKTVKKRPQRAES*N-C(Sx)-FA-Z (SEQ ID NO: 3610), X-KKKTVKKRPQRAES*N-C(Sx)-FS-Z (SEQ ID NO: 3611), X-KKKTVKKRPQRAESD-C(Sx)-FS*-Z (SEQ ID NO: 3612), X-KKKTVKKRPQRAESN-C(Sx)-FS*-Z (SEQ ID NO: 3613), X-KKKTVKKRPQRAHS*D-C(Sx)-FA-Z (SEQ ID NO: 3614), X-KKKTVKKRPQRAHS*D-C(Sx)-FS-Z (SEQ ID NO: 3615), X-KKKTVKKRPQRAHS*N-C(Sx)-FA-Z (SEQ ID NO: 3616), X-KKKTVKKRPQRAHS*N-C(Sx)-FS-Z (SEQ ID NO: 3617), X-KKKTVKKRPQRAHSD-C(Sx)-FS*-Z (SEQ ID NO: 3618), X-KKKTVKKRPQRAHSN-C(Sx)-FS*-Z (SEQ ID NO: 3619), X-KKKTVKKRPQRATS*D-C(Sx)-FA-Z (SEQ ID NO: 3620), X-KKKTVKKRPQRATS*D-C(Sx)-FS-Z (SEQ ID NO: 3621), X-KKKTVKKRPQRATS*N-C(Sx)-FA-Z (SEQ ID NO: 3622), X-KKKTVKKRPQRATS*N-C(Sx)-FS-Z (SEQ ID NO: 3623), X-KKKTVKKRPQRATSD-C(Sx)-FS*-Z (SEQ ID NO: 3624), X-KKKTVKKRPQRATSN-C(Sx)-FS*-Z (SEQ ID NO: 3625), and X-KKKTVNQSLLS*P-C(Sx)-VLEVD-Z (SEQ ID NO: 3626); wherein X— is H or acetyl; and Z is OH or NH2.

In some embodiments, the metal-binding compound of the present invention is selected from the group consisting of: X-KKKVLTQ[Nle]GSPS*I-C(Sx)-SS[pS]VS-Z (SEQ ID NO: 3627), X-KKKVLTQ[Nle]GSPSI-C(Sx)-SS*[pS]VS-Z (SEQ ID NO: 3628), X-KKKVPNGA-C(Sx)-SS*PVGNGFV-Z (SEQ ID NO: 3629), X-KKKVPNGAGSS*P-C(Sx)-GNGFV-Z (SEQ ID NO: 3630), X-KKKVSGQLID[Nle]M-C(Sx)-NS*FVGTRSY-Z (SEQ ID NO: 3631), X-KKKVSGQLIDAM-C(Sx)-NS*FVGTRSY-Z (SEQ ID NO: 3632), X-KKKVSGQLIDDM-C(Sx)-NS*FVGTRSY-Z (SEQ ID NO: 3633), X-KKKVSGQLIDEM-C(Sx)-NS*FVGTRSY-Z (SEQ ID NO: 3634), X-KKKVSGQLIDFM-C(Sx)-NS*FVGTRSY-Z (SEQ ID NO: 3635), X-KKKVSGQLIDGM-C(Sx)-NS*FVGTRSY-Z (SEQ ID NO: 3636), X-KKKVSGQLIDHM-C(Sx)-NS*FVGTRSY-Z (SEQ ID NO: 3637), X-KKKVSGQLIDIM-C(Sx)-NS*FVGTRSY-Z (SEQ ID NO: 3638), X-KKKVSGQLIDKM-C(Sx)-NS*FVGTRSY-Z (SEQ ID NO: 3639), X-KKKVSGQLIDLM-C(Sx)-NS*FVGTRSY-Z (SEQ ID NO: 3640), X-KKKVSGQLIDMM-C(Sx)-NS*FVGTRSY-Z (SEQ ID NO: 3641), X-KKKVSGQLIDNM-C(Sx)-NS*FVGTRSY-Z (SEQ ID NO: 3642), X-KKKVSGQLIDPM-C(Sx)-NS*FVGTRSY-Z (SEQ ID NO: 3643), X-KKKVSGQLIDQM-C(Sx)-NS*FVGTRSY-Z (SEQ ID NO: 3644), X-KKKVSGQLIDRM-C(Sx)-NS*FVGTRSY-Z (SEQ ID NO: 3645), X-KKKVSGQLIDS*[Nle]-C(Sx)-NSFVGTRSY-Z (SEQ ID NO: 3646), X-KKKVSGQLIDS*[Nle]-C(Sx)-NSFVG-Z (SEQ ID NO: 3647), X-KKKVSGQLIDS*M-C(Sx)-NAFVGTRSY-Z (SEQ ID NO: 3648), X-KKKVSGQLIDS*M-C(Sx)-NDFVGTRSY-Z (SEQ ID NO: 3649), X-KKKVSGQLIDS*M-C(Sx)-NEFVGTRSY-Z (SEQ ID NO: 3650), X-KKKVSGQLIDS*M-C(Sx)-NFFVGTRSY-Z (SEQ ID NO: 3651), X-KKKVSGQLIDS*M-C(Sx)-NIFVGTRSY-Z (SEQ ID NO: 3652), X-KKKVSGQLIDS*M-C(Sx)-NPFVGTRSY-Z (SEQ ID NO: 3653), X-KKKVSGQLIDS*M-C(Sx)-NS*FVGTRSY-Z (SEQ ID NO: 3654), X-KKKVSGQLIDS*M-C(Sx)-NSFVGTRS-Z (SEQ ID NO: 3655), X-KKKVSGQLIDS[Nle]-C(Sx)-NS*FVGTRSY-Z (SEQ ID NO: 3656), X-KKKVSGQLIDS[Nle]-C(Sx)-NS*FVG-Z (SEQ ID NO: 3657), X-KKKVSGQLIDSMANS*F-C(Sx)-GTRSY-Z (SEQ ID NO: 1006), X-KKKVSGQLIDSMANSF-C(Sx)-GT*RSY-Z (SEQ ID NO: 1196), X-KKKVSGQLIDSM-C(Sx)-NS*FVGTRS-Z (SEQ ID NO: 3658), X-KKKVSGQLIDVM-C(Sx)-NS*FVGTRSY-Z (SEQ ID NO: 3659), X-KKKVSGQLIDWM-C(Sx)-NS*FVGTRSY-Z (SEQ ID NO: 3660), X-KKKVSRSGLYRSPS*[Nle]-C(Sx)-ENLNRPR-Z (SEQ ID NO: 3661), X-KKKVSRSGLYRSPS*M-C(Sx)-ENLNRP-Z (SEQ ID NO: 3662), X-KKKVSRSGLYRSPS*M-C(Sx)-ENLNR-Z (SEQ ID NO: 3663), X-KKLGEDQAEEISDDLL-C(Sx)-DS*LSDEDE-Z (SEQ ID NO: 3664), X-KKLGEDQAEEISDDLLEDS*L-C(Sx)-DEDE-Z (SEQ ID NO: 3665), X-KKLNR-C(Sx)-LS*FAEG-Z (SEQ ID NO: 3666), X-KKLNR-C(Sx)-LS*FAEPG-Z (SEQ ID NO: 3667), X-KKLPQRAT*S-C(Sx)-VFAMF-Z (SEQ ID NO: 3668), X-KKLSTPV-C(Sx)-LS*PGPQKP-Z (SEQ ID NO: 3669), X-KKLSTPVVLS*P-C(Sx)-PQKP-Z (SEQ ID NO: 3670), and X-KKLSTPVVLS*PG-C(Sx)-QKP-Z (SEQ ID NO: 3671); wherein X is H or acetyl; and Z is OH or NH2.

In some embodiments, the metal-binding compound of the present invention is selected from the group consisting of: X-KKNPQRAT*S-C(Sx)-VFAMF-Z (SEQ ID NO: 3672), X-KKNVH[Nle]V-C(Sx)-TT*LPVDSRKK-Z (SEQ ID NO: 3673), X-KKPPQRAT*S-C(Sx)-VFAMF-Z (SEQ ID NO: 3674), X-KKPSVNPSIS*P-C(Sx)-HGVAR-Z (SEQ ID NO: 3675), X-KKQPQRAT*S-C(Sx)-VFAMF-Z (SEQ ID NO: 3676), X-KKR[Nle]QRAT*S-C(Sx)-VFAMF-Z (SEQ ID NO: 3677), X-KKRAA-C(Sx)-AT*SDVFA-Z (SEQ ID NO: 3678), X-KKRAARAT*S-C(Sx)-VFA-Z (SEQ ID NO: 3679), X-KKRAARATS*D-C(Sx)-FA-Z (SEQ ID NO: 3680), X-KKRAAR-C(Sx)-TS*DVFA-Z (SEQ ID NO: 3681), X-KKRDQRAT*S-C(Sx)-VFAMF-Z (SEQ ID NO: 3682), X-KKREQRAT*S-C(Sx)-VFAMF-Z (SEQ ID NO: 3683), X-KKRFQRAT*S-C(Sx)-VFAMF-Z (SEQ ID NO: 3684), X-KKRGQRAT*S-C(Sx)-VFAMF-Z (SEQ ID NO: 3685), X-KKRHQRAT*S-C(Sx)-VFAMF-Z (SEQ ID NO: 3686), X-KKRIQRAT*S-C(Sx)-VFAMF-Z (SEQ ID NO: 3687), X-KKRKQRAT*S-C(Sx)-VFAMF-Z (SEQ ID NO: 3688), X-KKRLQRAT*S-C(Sx)-VFAMF-Z (SEQ ID NO: 3689), X-KKRNQRAT*S-C(Sx)-VFAMF-Z (SEQ ID NO: 3690), X-KKRP[Nle]RAT*S-C(Sx)-VFAMF-Z (SEQ ID NO: 3691), X-KKRPDRAT*S-C(Sx)-VFAMF-Z (SEQ ID NO: 3692), X-KKRPERAT*S-C(Sx)-VFAMF-Z (SEQ ID NO: 3693), X-KKRPFRAT*S-C(Sx)-VFAMF-Z (SEQ ID NO: 3694), X-KKRPGRAT*S-C(Sx)-VFAMF-Z (SEQ ID NO: 3695), X-KKRPHRAT*S-C(Sx)-VFAMF-Z (SEQ ID NO: 3696), X-KKRPIRAT*S-C(Sx)-VFAMF-Z (SEQ ID NO: 3697), X-KKRPKRAT*S-C(Sx)-VFAMF-Z (SEQ ID NO: 3698), X-KKRPLRAT*S-C(Sx)-VFAMF-Z (SEQ ID NO: 3699), X-KKRPNRAT*S-C(Sx)-VFAMF-Z (SEQ ID NO: 3700), X-KKRPPRAT*S-C(Sx)-VFAMF-Z (SEQ ID NO: 3701), X-KKRPQ[Nle]AT*S-C(Sx)-VFAMF-Z (SEQ ID NO: 3702), X-KKRPQDAT*S-C(Sx)-VFAMF-Z (SEQ ID NO: 3703), X-KKRPQEAT*S-C(Sx)-VFAMF-Z (SEQ ID NO: 3704), X-KKRPQFAT*S-C(Sx)-VFAMF-Z (SEQ ID NO: 3705), X-KKRPQGAT*S-C(Sx)-VFAMF-Z (SEQ ID NO: 3706), X-KKRPQHAT*S-C(Sx)-VFAMF-Z (SEQ ID NO: 3707), X-KKRPQIAT*S-C(Sx)-VFAMF-Z (SEQ ID NO: 3708), X-KKRPQKAT*S-C(Sx)-VFAMF-Z (SEQ ID NO: 3709), X-KKRPQLAT*S-C(Sx)-VFAMF-Z (SEQ ID NO: 3710), X-KKRPQNAT*S-C(Sx)-VFAMF-Z (SEQ ID NO: 3711), X-KKRPQPAT*S-C(Sx)-VFAMF-Z (SEQ ID NO: 3712), X-KKRPQQAT*S-C(Sx)-VFAMF-Z (SEQ ID NO: 3713), X-KKRPQR[Nle]T*S-C(Sx)-VFAMF-Z (SEQ ID NO: 3714), X-KKRPQRAT*[Nle]-C(Sx)-VFAMF-Z (SEQ ID NO: 3715), X-KKRPQRAT*D-C(Sx)-VFAMF-Z (SEQ ID NO: 3716), X-KKRPQRAT*E-C(Sx)-VFAMF-Z (SEQ ID NO: 3717), X-KKRPQRAT*F-C(Sx)-VFAMF-Z (SEQ ID NO: 3718), X-KKRPQRAT*G-C(Sx)-VFAMF-Z (SEQ ID NO: 3719), X-KKRPQRAT*H-C(Sx)-VFAMF-Z (SEQ ID NO: 3720), X-KKRPQRAT*I-C(Sx)-VFAMF-Z (SEQ ID NO: 3721), X-KKRPQRAT*K-C(Sx)-VFAMF-Z (SEQ ID NO: 3722), X-KKRPQRAT*L-C(Sx)-VFAMF-Z (SEQ ID NO: 3723), X-KKRPQRAT*N-C(Sx)-VFAMF-Z (SEQ ID NO: 3724), X-KKRPQRAT*P-C(Sx)-VFAMF-Z (SEQ ID NO: 3725), X-KKRPQRAT*Q-C(Sx)-VFAMF-Z (SEQ ID NO: 3726), and X-KKRPQRAT*R-C(Sx)-VFAMF-Z (SEQ ID NO: 3727); wherein X— is H or acetyl; and Z is OH or NH2.

In some embodiments, the metal-binding compound of the present invention is selected from the group consisting of: X-KKRPQRAT*S-C(Sx)-[Nle]FAMF-Z (SEQ ID NO: 3728), X-KKRPQRAT*S-C(Sx)-DFAMF-Z (SEQ ID NO: 3729), X-KKRPQRAT*S-C(Sx)-EFAMF-Z (SEQ ID NO: 3730), X-KKRPQRAT*S-C(Sx)-FFAMF-Z (SEQ ID NO: 3731), X-KKRPQRAT*S-C(Sx)-GFAMF-Z (SEQ ID NO: 3732), X-KKRPQRAT*S-C(Sx)-HFAMF-Z (SEQ ID NO: 3733), X-KKRPQRAT*S-C(Sx)-IFAMF-Z (SEQ ID NO: 3734), X-KKRPQRAT*S-C(Sx)-KFAMF-Z (SEQ ID NO: 3735), X-KKRPQRAT*S-C(Sx)-LFAMF-Z (SEQ ID NO: 3736), X-KKRPQRAT*S-C(Sx)-NFAMF-Z (SEQ ID NO: 3737), X-KKRPQRAT*S-C(Sx)-PFAMF-Z (SEQ ID NO: 3738), X-KKRPQRAT*S-C(Sx)-QFAMF-Z (SEQ ID NO: 3739), X-KKRPQRAT*S-C(Sx)-RFAMF-Z (SEQ ID NO: 3740), X-KKRPQRAT*S-C(Sx)-V[Nle]AMF-Z (SEQ ID NO: 3741), X-KKRPQRAT*S-C(Sx)-VDAMF-Z (SEQ ID NO: 3742), X-KKRPQRAT*S-C(Sx)-VEAMF-Z (SEQ ID NO: 3743), X-KKRPQRAT*S-C(Sx)-VF[Nle]MF-Z (SEQ ID NO: 3744), X-KKRPQRAT*S-C(Sx)-VFADF-Z (SEQ ID NO: 3745), X-KKRPQRAT*S-C(Sx)-VFAEF-Z (SEQ ID NO: 3746), X-KKRPQRAT*S-C(Sx)-VFAFF-Z (SEQ ID NO: 3747), X-KKRPQRAT*S-C(Sx)-VFAGF-Z (SEQ ID NO: 3748), X-KKRPQRAT*S-C(Sx)-VFAHF-Z (SEQ ID NO: 3749), X-KKRPQRAT*S-C(Sx)-VFAIF-Z (SEQ ID NO: 3750), X-KKRPQRAT*S-C(Sx)-VFAKF-Z (SEQ ID NO: 3751), X-KKRPQRAT*S-C(Sx)-VFALF-Z (SEQ ID NO: 3752), X-KKRPQRAT*S-C(Sx)-VFAM[Nle]-Z (SEQ ID NO: 3753), X-KKRPQRAT*S-C(Sx)-VFAMD-Z (SEQ ID NO: 3754), X-KKRPQRAT*S-C(Sx)-VFAME-Z (SEQ ID NO: 3755), X-KKRPQRAT*S-C(Sx)-VFAMG-Z (SEQ ID NO: 3756), X-KKRPQRAT*S-C(Sx)-VFAMH-Z (SEQ ID NO: 3757), X-KKRPQRAT*S-C(Sx)-VFAMI-Z (SEQ ID NO: 3758), X-KKRPQRAT*S-C(Sx)-VFAMK-Z (SEQ ID NO: 3759), X-KKRPQRAT*S-C(Sx)-VFAML-Z (SEQ ID NO: 3760), X-KKRPQRAT*S-C(Sx)-VFAMN-Z (SEQ ID NO: 3761), X-KKRPQRAT*S-C(Sx)-VFAMP-Z (SEQ ID NO: 3762), X-KKRPQRAT*S-C(Sx)-VFAMQ-Z (SEQ ID NO: 3763), X-KKRPQRAT*S-C(Sx)-VFAMR-Z (SEQ ID NO: 3764), X-KKRPQRAT*S-C(Sx)-VFAMV-Z (SEQ ID NO: 3765), X-KKRPQRAT*S-C(Sx)-VFAMW-Z (SEQ ID NO: 3766), X-KKRPQRAT*S-C(Sx)-VFANF-Z (SEQ ID NO: 3767), X-KKRPQRAT*S-C(Sx)-VFAPF-Z (SEQ ID NO: 3768), X-KKRPQRAT*S-C(Sx)-VFAQF-Z (SEQ ID NO: 3769), X-KKRPQRAT*S-C(Sx)-VFARF-Z (SEQ ID NO: 3770), X-KKRPQRAT*S-C(Sx)-VFAVF-Z (SEQ ID NO: 3771), X-KKRPQRAT*S-C(Sx)-VFAWF-Z (SEQ ID NO: 3772), X-KKRPQRAT*S-C(Sx)-VFDMF-Z (SEQ ID NO: 3773), X-KKRPQRAT*S-C(Sx)-VFEMF-Z (SEQ ID NO: 3774), X-KKRPQRAT*S-C(Sx)-VFFMF-Z (SEQ ID NO: 3775), X-KKRPQRAT*S-C(Sx)-VFGMF-Z (SEQ ID NO: 3776), X-KKRPQRAT*S-C(Sx)-VFHMF-Z (SEQ ID NO: 3777), X-KKRPQRAT*S-C(Sx)-VFIMF-Z (SEQ ID NO: 3778), X-KKRPQRAT*S-C(Sx)-VFKMF-Z (SEQ ID NO: 3779), X-KKRPQRAT*S-C(Sx)-VFLMF-Z (SEQ ID NO: 3780), X-KKRPQRAT*S-C(Sx)-VFNMF-Z (SEQ ID NO: 3781), X-KKRPQRAT*S-C(Sx)-VFPMF-Z (SEQ ID NO: 3782), X-KKRPQRAT*S-C(Sx)-VFQMF-Z (SEQ ID NO: 3783), X-KKRPQRAT*S-C(Sx)-VFRMF-Z (SEQ ID NO: 3784), X-KKRPQRAT*S-C(Sx)-VFVMF-Z (SEQ ID NO: 3785), X-KKRPQRAT*S-C(Sx)-VFWMF-Z (SEQ ID NO: 3786), X-KKRPQRAT*S-C(Sx)-VGAMF-Z (SEQ ID NO: 3787), X-KKRPQRAT*S-C(Sx)-VHAMF-Z (SEQ ID NO: 3788), X-KKRPQRAT*S-C(Sx)-VIAMF-Z (SEQ ID NO: 3789), X-KKRPQRAT*S-C(Sx)-VKAMF-Z (SEQ ID NO: 3790), X-KKRPQRAT*S-C(Sx)-VLAMF-Z (SEQ ID NO: 3791), X-KKRPQRAT*S-C(Sx)-VNAMF-Z (SEQ ID NO: 3792), X-KKRPQRAT*S-C(Sx)-VPAMF-Z (SEQ ID NO: 3793), X-KKRPQRAT*S-C(Sx)-VQAMF-Z (SEQ ID NO: 3794), X-KKRPQRAT*S-C(Sx)-VRAMF-Z (SEQ ID NO: 3795), X-KKRPQRAT*S-C(Sx)-VVAMF-Z (SEQ ID NO: 3796), X-KKRPQRAT*S-C(Sx)-VWAMF-Z (SEQ ID NO: 3797), X-KKRPQRAT*S-C(Sx)-WFAMF-Z (SEQ ID NO: 3798), X-KKRPQRAT*V-C(Sx)-VFAMF-Z (SEQ ID NO: 3799), and X-KKRPQRAT*W-C(Sx)-VFAMF-Z (SEQ ID NO: 3800); wherein X is H or acetyl; and Z is OH or NH2.

In some embodiments, the metal-binding compound of the present invention is selected from the group consisting of: X-KKRPQR-C(Sx)-YS*NVF-Z (SEQ ID NO: 3801), X-KKRPQRDT*S-C(Sx)-VFAMF-Z (SEQ ID NO: 3802), X-KKRPQRET*S-C(Sx)-VFAMF-Z (SEQ ID NO: 3803), X-KKRPQRFT*S-C(Sx)-VFAMF-Z (SEQ ID NO: 3804), X-KKRPQRGT*S-C(Sx)-VFAMF-Z (SEQ ID NO: 3805), X-KKRPQRHT*S-C(Sx)-VFAMF-Z (SEQ ID NO: 3806), X-KKRPQRIT*S-C(Sx)-VFAMF-Z (SEQ ID NO: 3807), X-KKRPQRKT*S-C(Sx)-VFAMF-Z (SEQ ID NO: 3808), X-KKRPQRLT*S-C(Sx)-VFAMF-Z (SEQ ID NO: 3809), X-KKRPQRNT*S-C(Sx)-VFAMF-Z (SEQ ID NO: 3810), X-KKRPQRPT*S-C(Sx)-VFAMF-Z (SEQ ID NO: 3811), X-KKRPQRQT*S-C(Sx)-VFAMF-Z (SEQ ID NO: 3812), X-KKRPQRRT*S-C(Sx)-VFAMF-Z (SEQ ID NO: 3813), X-KKRPQRRYS*N-C(Sx)-F-Z (SEQ ID NO: 3814), X-KKRPQRVT*S-C(Sx)-VFAMF-Z (SEQ ID NO: 3815), X-KKRPQRWT*S-C(Sx)-VFAMF-Z (SEQ ID NO: 3816), X-KKRPQVAT*S-C(Sx)-VFAMF-Z (SEQ ID NO: 3817), X-KKRPQWAT*S-C(Sx)-VFAMF-Z (SEQ ID NO: 3818), X-KKRPRRAT*S-C(Sx)-VFAMF-Z (SEQ ID NO: 3819), X-KKRPVRAT*S-C(Sx)-VFAMF-Z (SEQ ID NO: 3820), X-KKRPWRAT*S-C(Sx)-VFAMF-Z (SEQ ID NO: 3821), X-KKRQQRAT*S-C(Sx)-VFAMF-Z (SEQ ID NO: 3822), X-KKRRQRAT*S-C(Sx)-VFAMF-Z (SEQ ID NO: 3823), X-KKRVQRAT*S-C(Sx)-VFAMF-Z (SEQ ID NO: 3824), X-KKRWQRAT*S-C(Sx)-VFAMF-Z (SEQ ID NO: 3825), X-KKS*R-C(Sx)-DY[Nle]T[Nle]QIG-Z (SEQ ID NO: 3826), X-KKVPQRAT*S-C(Sx)-VFAMF-Z (SEQ ID NO: 3827), X-KKVSRSGLYRSPS*[Nle]-C(Sx)-ENLNRP-Z (SEQ ID NO: 3828), X-KKVSRSGLYRSPS*[Nle]-C(Sx)-ENLNR-Z (SEQ ID NO: 3829), X-KKVSRSGLYRSPS*M-C(Sx)-ENLNRPR-Z (SEQ ID NO: 3830), X-KKVSRSGLYRSPS*M-C(Sx)-ENLNRP-Z (SEQ ID NO: 3831), X-KKVSRSGLYRSPS*M-C(Sx)-ENLNR-Z (SEQ ID NO: 3832), X-KKWPQRAT*S-C(Sx)-VFAMF-Z (SEQ ID NO: 3833), X-KLKTTKKRPQRATS*N-C(Sx)-FS-Z (SEQ ID NO: 3834), X-KLKTTKKRPQRATSN-C(Sx)-FS*-Z (SEQ ID NO: 3835), X-KLLR-C(Sx)-SS*RRIRR-Z (SEQ ID NO: 3836), X-KLPWWR-C(Sx)-YT*WVVERDVNTKQR-Z (SEQ ID NO: 3837), X-KLRPQRAT*S-C(Sx)-VFAMF-Z (SEQ ID NO: 3838), X-KNDG-C(Sx)-RT*RSKGTLRYMSPE-Z (SEQ ID NO: 3839), X-KNDGKRT*R-C(Sx)-KGTLRYMSPE-Z (SEQ ID NO: 3840), X-KNDGKRTRS-C(Sx)-GT*LRYMSPE-Z (SEQ ID NO: 3841), X-KNDGKRTRSKGT*L-C(Sx)-YMSPE-Z (SEQ ID NO: 3842), X-KNIVT-C(Sx)-RT*PPPSQGK-Z (SEQ ID NO: 1059), X-KNIVTPRT*P-C(Sx)-PSQGK-Z (SEQ ID NO: 1058), X-KNIVTPRTP-C(Sx)-PS*QGK-Z (SEQ ID NO: 3843), X-KNKTTKKRPQRATS*N-C(Sx)-FS-Z (SEQ ID NO: 3844), -KNKTTKKRPQRATSN-C(Sx)-FS*-Z (SEQ ID NO: 3845); wherein X— is H or acetyl; and Z is OH or NH2.

In some embodiments, the metal-binding compound of the present invention is selected from the group consisting of: X-KNRPQRAT*S-C(Sx)-VFAMF-Z (SEQ ID NO: 3846), X-KPARKKRYT*V-C(Sx)-GNPYWM-Z (SEQ ID NO: 3847), X-KPKKKKKRPQRADS*D-C(Sx)-FA-Z (SEQ ID NO: 3848), X-KPKKKKKRPQRADS*D-C(Sx)-FS-Z (SEQ ID NO: 3849), X-KPKKKKKRPQRADS*N-C(Sx)-FA-Z (SEQ ID NO: 3850), X-KPKKKKKRPQRADS*N-C(Sx)-FS-Z (SEQ ID NO: 3851), X-KPKKKKKRPQRADSD-C(Sx)-FS*-Z (SEQ ID NO: 3852), X-KPKKKKKRPQRADSN-C(Sx)-FS*-Z (SEQ ID NO: 3853), X-KPKKKKKRPQRAES*D-C(Sx)-FA-Z (SEQ ID NO: 3854), X-KPKKKKKRPQRAES*D-C(Sx)-FS-Z (SEQ ID NO: 3855), X-KPKKKKKRPQRAES*N-C(Sx)-FA-Z (SEQ ID NO: 3856), X-KPKKKKKRPQRAES*N-C(Sx)-FS-Z (SEQ ID NO: 3857), X-KPKKKKKRPQRAESD-C(Sx)-FS*-Z (SEQ ID NO: 3858), X-KPKKKKKRPQRAESN-C(Sx)-FS*-Z (SEQ ID NO: 3859), X-KPKKKKKRPQRAHS*D-C(Sx)-FA-Z (SEQ ID NO: 3860), X-KPKKKKKRPQRAHS*D-C(Sx)-FS-Z (SEQ ID NO: 3861), X-KPKKKKKRPQRAHS*N-C(Sx)-FA-Z (SEQ ID NO: 3862), X-KPKKKKKRPQRAHS*N-C(Sx)-FS-Z (SEQ ID NO: 3863), X-KPKKKKKRPQRAHSD-C(Sx)-FS*-Z (SEQ ID NO: 3864), X-KPKKKKKRPQRAHSN-C(Sx)-FS*-Z (SEQ ID NO: 3865), X-KPKKKKKRPQRATS*D-C(Sx)-FA-Z (SEQ ID NO: 3866), X-KPKKKKKRPQRATS*D-C(Sx)-FS-Z (SEQ ID NO: 3867), X-KPKKKKKRPQRATS*N-C(Sx)-FA-Z (SEQ ID NO: 3868), X-KPKKKKKRPQRATS*N-C(Sx)-FS-Z (SEQ ID NO: 3869), X-KPKKKKKRPQRATSD-C(Sx)-FS*-Z (SEQ ID NO: 3870), X-KPKKKKKRPQRATSN-C(Sx)-FS*-Z (SEQ ID NO: 3871), X-KPKKRKKRPQRADS*D-C(Sx)-FA-Z (SEQ ID NO: 3872), X-KPKKRKKRPQRADS*D-C(Sx)-FS-Z (SEQ ID NO: 3873), X-KPKKRKKRPQRADS*N-C(Sx)-FA-Z (SEQ ID NO: 3874), X-KPKKRKKRPQRADS*N-C(Sx)-FS-Z (SEQ ID NO: 3875), X-KPKKRKKRPQRADSD-C(Sx)-FS*-Z (SEQ ID NO: 3876), X-KPKKRKKRPQRADSN-C(Sx)-FS*-Z (SEQ ID NO: 3877), X-KPKKRKKRPQRAES*D-C(Sx)-FA-Z (SEQ ID NO: 3878), X-KPKKRKKRPQRAES*D-C(Sx)-FS-Z (SEQ ID NO: 3879), X-KPKKRKKRPQRAES*N-C(Sx)-FA-Z (SEQ ID NO: 3880), X-KPKKRKKRPQRAES*N-C(Sx)-FS-Z (SEQ ID NO: 3881), X-KPKKRKKRPQRAESD-C(Sx)-FS*-Z (SEQ ID NO: 3882), X-KPKKRKKRPQRAESN-C(Sx)-FS*-Z (SEQ ID NO: 3883), X-KPKKRKKRPQRAHS*D-C(Sx)-FA-Z (SEQ ID NO: 3884), X-KPKKRKKRPQRAHS*D-C(Sx)-FS-Z (SEQ ID NO: 3885), X-KPKKRKKRPQRAHS*N-C(Sx)-FA-Z (SEQ ID NO: 3886), X-KPKKRKKRPQRAHS*N-C(Sx)-FS-Z (SEQ ID NO: 3887), X-KPKKRKKRPQRAHSD-C(Sx)-FS*-Z (SEQ ID NO: 3888), X-KPKKRKKRPQRAHSN-C(Sx)-FS*-Z (SEQ ID NO: 3889), X-KPKKRKKRPQRATS*D-C(Sx)-FA-Z (SEQ ID NO: 3890), X-KPKKRKKRPQRATS*D-C(Sx)-FS-Z (SEQ ID NO: 3891), and X-KPKKRKKRPQRATS*N-C(Sx)-FA-Z (SEQ ID NO: 3892); wherein X is H or acetyl; and Z is OH or NH2.

In some embodiments, the metal-binding compound of the present invention is selected from the group consisting of: X-KPKKRKKRPQRATS*N-C(Sx)-FS-Z (SEQ ID NO: 3893), X-KPKKRKKRPQRATSD-C(Sx)-FS*-Z (SEQ ID NO: 3894), X-KPKKRKKRPQRATSN-C(Sx)-FS*-Z (SEQ ID NO: 3895), X-KPKKTKKRPQRADS*D-C(Sx)-FA-Z (SEQ ID NO: 3896), X-KPKKTKKRPQRADS*D-C(Sx)-FS-Z (SEQ ID NO: 3897), X-KPKKTKKRPQRADS*N-C(Sx)-FA-Z (SEQ ID NO: 3898), X-KPKKTKKRPQRADS*N-C(Sx)-FS-Z (SEQ ID NO: 3899), X-KPKKTKKRPQRADSD-C(Sx)-FS*-Z (SEQ ID NO: 3900), X-KPKKTKKRPQRADSN-C(Sx)-FS*-Z (SEQ ID NO: 3901), X-KPKKTKKRPQRAES*D-C(Sx)-FA-Z (SEQ ID NO: 3902), X-KPKKTKKRPQRAES*D-C(Sx)-FS-Z (SEQ ID NO: 3903), X-KPKKTKKRPQRAES*N-C(Sx)-FA-Z (SEQ ID NO: 3904), X-KPKKTKKRPQRAES*N-C(Sx)-FS-Z (SEQ ID NO: 3905), X-KPKKTKKRPQRAESD-C(Sx)-FS*-Z (SEQ ID NO: 3906), X-KPKKTKKRPQRAESN-C(Sx)-FS*-Z (SEQ ID NO: 3907), X-KPKKTKKRPQRAHS*D-C(Sx)-FA-Z (SEQ ID NO: 3908), X-KPKKTKKRPQRAHS*D-C(Sx)-FS-Z (SEQ ID NO: 3909), X-KPKKTKKRPQRAHS*N-C(Sx)-FA-Z (SEQ ID NO: 3910), X-KPKKTKKRPQRAHS*N-C(Sx)-FS-Z (SEQ ID NO: 3911), X-KPKKTKKRPQRAHSD-C(Sx)-FS*-Z (SEQ ID NO: 3912), X-KPKKTKKRPQRAHSN-C(Sx)-FS*-Z (SEQ ID NO: 3913), X-KPKKTKKRPQRATS*D-C(Sx)-FA-Z (SEQ ID NO: 3914), X-KPKKTKKRPQRATS*D-C(Sx)-FS-Z (SEQ ID NO: 3915), X-KPKKTKKRPQRATS*N-C(Sx)-FA-Z (SEQ ID NO: 3916), X-KPKKTKKRPQRATS*N-C(Sx)-FS-Z (SEQ ID NO: 3917), X-KPKKTKKRPQRATSD-C(Sx)-FS*-Z (SEQ ID NO: 3918), X-KPKKTKKRPQRATSN-C(Sx)-FS*-Z (SEQ ID NO: 3919), X-KPKKVKKRPQRADS*D-C(Sx)-FA-Z (SEQ ID NO: 3920), X-KPKKVKKRPQRADS*D-C(Sx)-FS-Z (SEQ ID NO: 3921), X-KPKKVKKRPQRADS*N-C(Sx)-FA-Z (SEQ ID NO: 3922), X-KPKKVKKRPQRADS*N-C(Sx)-FS-Z (SEQ ID NO: 3923), X-KPKKVKKRPQRADSD-C(Sx)-FS*-Z (SEQ ID NO: 3924), X-KPKKVKKRPQRADSN-C(Sx)-FS*-Z (SEQ ID NO: 3925), X-KPKKVKKRPQRAES*D-C(Sx)-FA-Z (SEQ ID NO: 3926), X-KPKKVKKRPQRAES*D-C(Sx)-FS-Z (SEQ ID NO: 3927), X-KPKKVKKRPQRAES*N-C(Sx)-FA-Z (SEQ ID NO: 3928), X-KPKKVKKRPQRAES*N-C(Sx)-FS-Z (SEQ ID NO: 3929), X-KPKKVKKRPQRAESD-C(Sx)-FS*-Z (SEQ ID NO: 3930), X-KPKKVKKRPQRAESN-C(Sx)-FS*-Z (SEQ ID NO: 3931), X-KPKKVKKRPQRAHS*D-C(Sx)-FA-Z (SEQ ID NO: 3932), X-KPKKVKKRPQRAHS*D-C(Sx)-FS-Z (SEQ ID NO: 3933), X-KPKKVKKRPQRAHS*N-C(Sx)-FA-Z (SEQ ID NO: 3934), X-KPKKVKKRPQRAHS*N-C(Sx)-FS-Z (SEQ ID NO: 3935), X-KPKKVKKRPQRAHSD-C(Sx)-FS*-Z (SEQ ID NO: 3936), X-KPKKVKKRPQRAHSN-C(Sx)-FS*-Z (SEQ ID NO: 3937), X-KPKKVKKRPQRATS*D-C(Sx)-FA-Z (SEQ ID NO: 3938), X-KPKKVKKRPQRATS*D-C(Sx)-FS-Z (SEQ ID NO: 3939), X-KPKKVKKRPQRATS*N-C(Sx)-FA-Z (SEQ ID NO: 3940), X-KPKKVKKRPQRATS*N-C(Sx)-FS-Z (SEQ ID NO: 3941), X-KPKKVKKRPQRATSD-C(Sx)-FS*-Z (SEQ ID NO: 3942), X-KPKKVKKRPQRATSN-C(Sx)-FS*-Z (SEQ ID NO: 3943), and X-KPKTKKKRPQRADS*D-C(Sx)-FA-Z (SEQ ID NO: 3944); wherein X is H or acetyl; and Z is OH or NH2.

In some embodiments, the metal-binding compound of the present invention is selected from the group consisting of: X-KPKTKKKRPQRADS*D-C(Sx)-FS-Z (SEQ ID NO: 3945), X-KPKTKKKRPQRADS*N-C(Sx)-FA-Z (SEQ ID NO: 3946), X-KPKTKKKRPQRADS*N-C(Sx)-FS-Z (SEQ ID NO: 3947), X-KPKTKKKRPQRADSD-C(Sx)-FS*-Z (SEQ ID NO: 3948), X-KPKTKKKRPQRADSN-C(Sx)-FS*-Z (SEQ ID NO: 3949), X-KPKTKKKRPQRAES*D-C(Sx)-FA-Z (SEQ ID NO: 3950), X-KPKTKKKRPQRAES*D-C(Sx)-FS-Z (SEQ ID NO: 3951), X-KPKTKKKRPQRAES*N-C(Sx)-FA-Z (SEQ ID NO: 3952), X-KPKTKKKRPQRAES*N-C(Sx)-FS-Z (SEQ ID NO: 3953), X-KPKTKKKRPQRAESD-C(Sx)-FS*-Z (SEQ ID NO: 3954), X-KPKTKKKRPQRAESN-C(Sx)-FS*-Z (SEQ ID NO: 3955), X-KPKTKKKRPQRAHS*D-C(Sx)-FA-Z (SEQ ID NO: 3956), X-KPKTKKKRPQRAHS*D-C(Sx)-FS-Z (SEQ ID NO: 3957), X-KPKTKKKRPQRAHS*N-C(Sx)-FA-Z (SEQ ID NO: 3958), X-KPKTKKKRPQRAHS*N-C(Sx)-FS-Z (SEQ ID NO: 3959), X-KPKTKKKRPQRAHSD-C(Sx)-FS*-Z (SEQ ID NO: 3960), X-KPKTKKKRPQRAHSN-C(Sx)-FS*-Z (SEQ ID NO: 3961), X-KPKTKKKRPQRATS*D-C(Sx)-FA-Z (SEQ ID NO: 3962), X-KPKTKKKRPQRATS*D-C(Sx)-FS-Z (SEQ ID NO: 3963), X-KPKTKKKRPQRATS*N-C(Sx)-FA-Z (SEQ ID NO: 3964), X-KPKTKKKRPQRATS*N-C(Sx)-FS-Z (SEQ ID NO: 3965), X-KPKTKKKRPQRATSD-C(Sx)-FS*-Z (SEQ ID NO: 3966), X-KPKTKKKRPQRATSN-C(Sx)-FS*-Z (SEQ ID NO: 3967), X-KPKTRKKRPQRADS*D-C(Sx)-FA-Z (SEQ ID NO: 3968), X-KPKTRKKRPQRADS*D-C(Sx)-FS-Z (SEQ ID NO: 3969), X-KPKTRKKRPQRADS*N-C(Sx)-FA-Z (SEQ ID NO: 3970), X-KPKTRKKRPQRADS*N-C(Sx)-FS-Z (SEQ ID NO: 3971), X-KPKTRKKRPQRADSD-C(Sx)-FS*-Z (SEQ ID NO: 3972), and X-KPKTRKKRPQRADSN-C(Sx)-FS*-Z (SEQ ID NO: 3973); wherein X— is H or acetyl; and Z is OH or NH2.

In some embodiments, the metal-binding compound of the present invention is selected from the group consisting of: X-KPKTRKKRPQRAES*D-C(Sx)-FA-Z (SEQ ID NO: 3974), X-KPKTRKKRPQRAES*D-C(Sx)-FS-Z (SEQ ID NO: 3975), X-KPKTRKKRPQRAES*N-C(Sx)-FA-Z (SEQ ID NO: 3976), X-KPKTRKKRPQRAES*N-C(Sx)-FS-Z (SEQ ID NO: 3977), X-KPKTRKKRPQRAESD-C(Sx)-FS*-Z (SEQ ID NO: 3978), X-KPKTRKKRPQRAESN-C(Sx)-FS*-Z (SEQ ID NO: 3979), X-KPKTRKKRPQRAHS*D-C(Sx)-FA-Z (SEQ ID NO: 3980), X-KPKTRKKRPQRAHS*D-C(Sx)-FS-Z (SEQ ID NO: 3981), X-KPKTRKKRPQRAHS*N-C(Sx)-FA-Z (SEQ ID NO: 3982), X-KPKTRKKRPQRAHS*N-C(Sx)-FS-Z (SEQ ID NO: 3983), X-KPKTRKKRPQRAHSD-C(Sx)-FS*-Z (SEQ ID NO: 3984), X-KPKTRKKRPQRAHSN-C(Sx)-FS*-Z (SEQ ID NO: 3985), X-KPKTRKKRPQRATS*D-C(Sx)-FA-Z (SEQ ID NO: 3986), X-KPKTRKKRPQRATS*D-C(Sx)-FS-Z (SEQ ID NO: 3987), X-KPKTRKKRPQRATS*N-C(Sx)-FA-Z (SEQ ID NO: 3988), X-KPKTRKKRPQRATS*N-C(Sx)-FS-Z (SEQ ID NO: 3989), X-KPKTRKKRPQRATSD-C(Sx)-FS*-Z (SEQ ID NO: 3990), X-KPKTRKKRPQRATSN-C(Sx)-FS*-Z (SEQ ID NO: 3991), X-KPKTKKRPQRADS*D-C(Sx)-FA-Z (SEQ ID NO: 3992), X-KPKTTKKRPQRADS*D-C(Sx)-FS-Z (SEQ ID NO: 3993), X-KPKTITKKRPQRADS*N-C(Sx)-FA-Z (SEQ ID NO: 3994), X-KPKTTKKRPQRADS*N-C(Sx)-FS-Z (SEQ ID NO: 3995), X-KPKTTKKRPQRADSD-C(Sx)-FS*-Z (SEQ ID NO: 3996), X-KPKTTKKRPQRADSN-C(Sx)-FS*-Z (SEQ ID NO: 3997), X-KPKTRKKRPQRAES*D-C(Sx)-FA-Z (SEQ ID NO: 3998), X-KPKTKKRPQRAES*D-C(Sx)-FS-Z (SEQ ID NO: 3999), X-KPKTKKRPQRAES*N-C(Sx)-FA-Z (SEQ ID NO: 4000), X-KPKTTKKRPQRAES*N-C(Sx)-FS-Z (SEQ ID NO: 4001), X-KPKTTKKRPQRAESD-C(Sx)-FS*-Z (SEQ ID NO: 4002), X-KPKTFKKRPQRAESN-C(Sx)-FS*-Z (SEQ ID NO: 4003), X-KPKTITKKRPQRAHS*D-C(Sx)-FA-Z (SEQ ID NO: 4004), X-KPKTTKKRPQRAHS*D-C(Sx)-FS-Z (SEQ ID NO: 4005), X-KPKTITKKRPQRAHS*N-C(Sx)-FA-Z (SEQ ID NO: 4006), X-KPKTTKKRPQRAHS*N-C(Sx)-FS-Z (SEQ ID NO: 4007), X-KPKTTKKRPQRAHSD-C(Sx)-FS*-Z (SEQ ID NO: 4008), X-KPKTTKKRPQRAHSN-C(Sx)-FS*-Z (SEQ ID NO: 4009), X-KPKTTKKRPQRATS*D-C(Sx)-FA-Z (SEQ ID NO: 4010), X-KPKTKKRPQRATS*D-C(Sx)-FS-Z (SEQ ID NO: 4011), X-KPKTKKRPQRATS*N-C(Sx)-FA-Z (SEQ ID NO: 4012), X-KPKTRKKRPQRATS*N-C(Sx)-FS-Z (SEQ ID NO: 4013), X-KPKTTKKRPQRATSD-C(Sx)-FS*-Z (SEQ ID NO: 4014), X-KPKTFKKRPQRATSN-C(Sx)-FS*-Z (SEQ ID NO: 4015), X-KPKTVKKRPQRADS*D-C(Sx)-FA-Z (SEQ ID NO: 4016), X-KPKTVKKRPQRADS*D-C(Sx)-FS-Z (SEQ ID NO: 4017), X-KPKTVKKRPQRADS*N-C(Sx)-FA-Z (SEQ ID NO: 4018), X-KPKTVKKRPQRADS*N-C(Sx)-FS-Z (SEQ ID NO: 4019), X-KPKTVKKRPQRADSD-C(Sx)-FS*-Z (SEQ ID NO: 4020), X-KPKTVKKRPQRADSN-C(Sx)-FS*-Z (SEQ ID NO: 4021), X-KPKTVKKRPQRAES*D-C(Sx)-FA-Z (SEQ ID NO: 4022), X-KPKTVKKRPQRAES*D-C(Sx)-FS-Z (SEQ ID NO: 4023), X-KPKTVKKRPQRAES*N-C(Sx)-FA-Z (SEQ ID NO: 4024), X-KPKTVKKRPQRAES*N-C(Sx)-FS-Z (SEQ ID NO: 4025), X-KPKTVKKRPQRAESD-C(Sx)-FS*-Z (SEQ ID NO: 4026), X-KPKTVKKRPQRAESN-C(Sx)-FS*-Z (SEQ ID NO: 4027), X-KPKTVKKRPQRAHS*D-C(Sx)-FA-Z (SEQ ID NO: 4028), X-KPKTVKKRPQRAHS*D-C(Sx)-FS-Z (SEQ ID NO: 4029), X-KPKTVKKRPQRAHS*N-C(Sx)-FA-Z (SEQ ID NO: 4030), X-KPKTVKKRPQRAHS*N-C(Sx)-FS-Z (SEQ ID NO: 4031), X-KPKTVKKRPQRAHSD-C(Sx)-FS*-Z (SEQ ID NO: 4032), X-KPKTVKKRPQRAHSN-C(Sx)-FS*-Z (SEQ ID NO: 4033), X-KPKTVKKRPQRATS*D-C(Sx)-FA-Z (SEQ ID NO: 4034), X-KPKTVKKRPQRATS*D-C(Sx)-FS-Z (SEQ ID NO: 4035), X-KPKTVKKRPQRATS*N-C(Sx)-FA-Z (SEQ ID NO: 4036), X-KPKTVKKRPQRATS*N-C(Sx)-FS-Z (SEQ ID NO: 4037), X-KPKTVKKRPQRATSD-C(Sx)-FS*-Z (SEQ ID NO: 4038), X-KPKTVKKRPQRATSN-C(Sx)-FS*-Z (SEQ ID NO: 4039), X-KPRPQRAT*S-C(Sx)-VFAMF-Z (SEQ ID NO: 4040), X-KQREGFGRQS*M-C(Sx)-EKR-Z (SEQ ID NO: 4041), and X-KQRPQRAT*S-C(Sx)-VFAMF-Z (SEQ ID NO: 4042); wherein X— is H or acetyl; and Z is OH or NH2.

In some embodiments, the metal-binding compound of the present invention is selected from the group consisting of: X-KRKLP-C(Sx)-DT*PGQGLT-Z (SEQ ID NO: 1125), X-KRKTTKKRPQRATS*N-C(Sx)-FS-Z (SEQ ID NO: 4043), X-KRKTTKKRPQRATSN-C(Sx)-FS*-Z (SEQ ID NO: 4044), X-KRR-C(Sx)-AS*FRRR-Z (SEQ ID NO: 4045), X-KRRPQRAT*S-C(Sx)-VFAMF-Z (SEQ ID NO: 4046), X-KRRRLAS*L-C(Sx)-G-Z (SEQ ID NO: 4047), X-KRS*R-C(Sx)-SSFPPGTRK-Z (SEQ ID NO: 4048), X-KRSR-C(Sx)-SS*FPPGTRK-Z (SEQ ID NO: 4049), X-KS*N-C(Sx)-ESGDSQQPSQPSQ-Z (SEQ ID NO: 4050), X-KSN-C(Sx)-ES*GDSQQPSQPSQ-Z (SEQ ID NO: 4051), X-KSNEESGDS*Q-C(Sx)-PSQPSQQPSV-Z (SEQ ID NO: 4052), X-KSNEESGDSQ-C(Sx)-PS*QPSQQPSV-Z (SEQ ID NO: 4053), X-KSNEESGDSQQPSQPS*Q-C(Sx)-PSV-Z (SEQ ID NO: 4054), X-KSNEESGDSQQPSQPSQ-C(Sx)-PS*V-Z (SEQ ID NO: 4055), X-KTTKKRPQRATS*N-C(Sx)-FS-Z (SEQ ID NO: 1000), X-KTTKKRPQRATSN-C(Sx)-FS*-Z (SEQ ID NO: 4056), X-KVKTTKKRPQRATS*N-C(Sx)-FS-Z (SEQ ID NO: 4057), X-KVKTTKKRPQRATSN-C(Sx)-FS*-Z (SEQ ID NO: 4058), X-KVRPQRAT*S-C(Sx)-VFAMF-Z (SEQ ID NO: 4059), X-KVSRSGLYRSPS*[Nle]-C(Sx)-ENLNRP-Z (SEQ ID NO: 4060), X-KVSRSGLYRSPS*[Nle]-C(Sx)-ENLNR-Z (SEQ ID NO: 4061), X-KVSRSGLYRSPS*M-C(Sx)-ENLNRPR-Z (SEQ ID NO: 4062), X-KVSRSGLYRSPS*M-C(Sx)-ENLNRP-Z (SEQ ID NO: 4063), X-KVSRSGLYRSPS*M-C(Sx)-ENLNR-Z (SEQ ID NO: 4064), X-KWKTTKKRPQRATS*N-C(Sx)-FS-Z (SEQ ID NO: 4065), X-KWKTTKKRPQRATSN-C(Sx)-FS*-Z (SEQ ID NO: 4066), X-KWR[Nle][Nle][Nle]S*L-C(Sx)-QSRSSKS-Z (SEQ ID NO: 4067), X-KWR[Nle][Nle][Nle]SL-C(Sx)-QS*RSSKS-Z (SEQ ID NO: 4068), X-KWR[Nle][Nle]S*L-C(Sx)-QSRSSKS-Z (SEQ ID NO: 4069), X-KWR[Nle][Nle]SL-C(Sx)-QS*RSSKS-Z (SEQ ID NO: 4070), X-KWR-C(Sx)-[Nle]S*LSQSRSSKS-Z (SEQ ID NO: 4071), X-KWR-C(Sx)-MS*LSQSRSSKSAPE-Z (SEQ ID NO: 4072), X-KWR-C(Sx)-MS*LSQSRSSKS-Z (SEQ ID NO: 4073), X-KWRMMS*L-C(Sx)-QSRSSKS-Z (SEQ ID NO: 4074), X-KWRMMSL-C(Sx)-QS*RSSKS-Z (SEQ ID NO: 4075), X-KWRPQRAT*S-C(Sx)-VFAMF-Z (SEQ ID NO: 4076), X-LAGISRS*V-C(Sx)-VTVAY-Z (SEQ ID NO: 1083), X-LAKTTKKRPQRATS*N-C(Sx)-FS-Z (SEQ ID NO: 4077), and X-LAKTTKKRPQRATSN-C(Sx)-FS*-Z (SEQ ID NO: 4078); wherein X is H or acetyl; and Z is OH or NH2.

In some embodiments, the metal-binding compound of the present invention is selected from the group consisting of: X-LANPLMA-C(Sx)-GT*LTRRHQNGRF-Z (SEQ ID NO: 4079), X-LASFK[Nle]WS*K-C(Sx)-NNEEH-Z (SEQ ID NO: 4080), X-LASFK-C(Sx)-WS*KLNNEEH-Z (SEQ ID NO: 4081), X-L-C(Sx)-DS*LRRKAK-Z (SEQ ID NO: 4082), X-L-C(Sx)-LS*PGPF-Z (SEQ ID NO: 4083), X-LGGLRISS*D-C(Sx)-SSDIE-Z (SEQ ID NO: 1082), X-LGGLRISSD-C(Sx)-SS*DIE-Z (SEQ ID NO: 4084), X-LHQEDNDYI-C(Sx)-AS*LIK-Z (SEQ ID NO: 4085), X-LIDAT-C(Sx)-DT*PGAEDDE-Z (SEQ ID NO: 1043), X-LIDATGDT*P-C(Sx)-AEDDE-Z (SEQ ID NO: 1042), X-LIDATGDT*PG-C(Sx)-EDDE-Z (SEQ ID NO: 4086), X-LIDS*[Nle]-C(Sx)-NSFVGTRSYAKKK-Z (SEQ ID NO: 4087), X-LIDS[Nle]-C(Sx)-NS*FVGTRSYAKKK-Z (SEQ ID NO: 4088), X-LKKERLLDDRHDS*G-C(Sx)-DSMKDEEY-Z (SEQ ID NO: 4089), X-LKKERLLDDRHDSG-C(Sx)-DS*MKDEEY-Z (SEQ ID NO: 4090), X-LKKERLLDDRHDSGLDS*M-C(Sx)-DEEY-Z (SEQ ID NO: 4091), X-LKR-C(Sx)-AS*FKKFA-Z (SEQ ID NO: 4092), X-LKRPQRAT*S-C(Sx)-VFAMF-Z (SEQ ID NO: 4093), X-LLDDR-C(Sx)-DS*GLDSMKD-Z (SEQ ID NO: 4094), X-LLDDRHDS*G-C(Sx)-DSMKD-Z (SEQ ID NO: 4095), X-LLDDRHDSG-C(Sx)-DS*MKD-Z (SEQ ID NO: 4096), X-LLLR-C(Sx)-SS*RRIRR-Z (SEQ ID NO: 4097), X-LLPWWR-C(Sx)-YT*WVVERDVNTKQR-Z (SEQ ID NO: 4098), X-LLSNASAT*P-C(Sx)-GRRGR-Z (SEQ ID NO: 1072), X-LMLRLQDYE-C(Sx)-KT*KKA-Z (SEQ ID NO: 4099), X-LPGPPS*P-C(Sx)-SDAES-Z (SEQ ID NO: 1181), X-LQREGFGRQS*M-C(Sx)-EKR-Z (SEQ ID NO: 4100), X-LRELGQGS*F-C(Sx)-MVYEG-Z (SEQ ID NO: 4101), X-LRRAS*L-C(Sx)-AA-Z (SEQ ID NO: 4102), X-LRR-C(Sx)-AS*FRRR-Z (SEQ ID NO: 4103), X-LSPVAPLS*P-C(Sx)-RLQGP-Z (SEQ ID NO: 1108), X-LSRDLIMKEDYE-C(Sx)-VS*TKPTR-Z (SEQ ID NO: 4104), X-LSRDLIMKEDYE-C(Sx)-VS*TK-Z (SEQ ID NO: 4105), X-LVEKLPDS*P-C(Sx)-LAKKA-Z (SEQ ID NO: 1078), X-M-C(Sx)-LS*PGPF-Z (SEQ ID NO: 4106), X-NAKTTKKRPQRATS*N-C(Sx)-FS-Z (SEQ ID NO: 4107), X-NAKTTKKRPQRATSN-C(Sx)-FS*-Z (SEQ ID NO: 4108), X-NANPLMA-C(Sx)-GT*LTRRHQNGRF-Z (SEQ ID NO: 4109), X-N-C(Sx)-DS*LRRKAK-Z (SEQ ID NO: 4110), X-N-C(Sx)-LS*PGPF-Z (SEQ ID NO: 4111), X-NKEKKAVS*P-C(Sx)-LLTIT-Z (SEQ ID NO: 4112), X-NKEKK-C(Sx)-VS*PLLLTIT-Z (SEQ ID NO: 4113), X-NKRPQRAT*S-C(Sx)-VFAMF-Z (SEQ ID NO: 4114), X-NLLR-C(Sx)-SS*RRIRR-Z (SEQ ID NO: 4115), X-NLPWWR-C(Sx)-YT*WVVERDVNTKQR-Z (SEQ ID NO: 4116), X-NPQDSVGS*P-C(Sx)-SRVGE-Z (SEQ ID NO: 1084), X-NQREGFGRQS*M-C(Sx)-EKR-Z (SEQ ID NO: 4117), X-NQRKA-C(Sx)-RS*LAPRFDL-Z (SEQ ID NO: 4118), X-NQRKAKRS*L-C(Sx)-PRFDL-Z (SEQ ID NO: 4119), X-NR-C(Sx)-LS*FAEG-Z (SEQ ID NO: 4120), X-NR-C(Sx)-LS*FAEPG-Z (SEQ ID NO: 4121), X-NRR-C(Sx)-AS*FRRR-Z (SEQ ID NO: 4122), X-NSKYNAES*T-C(Sx)-RESQDT-Z (SEQ ID NO: 4123), X-NTIDL-C(Sx)-MS*PRTLDSL-Z (SEQ ID NO: 1029), X-NTIDLPMS*P-C(Sx)-TLDSL-Z (SEQ ID NO: 1028), and X-NVLAP-C(Sx)-PS*QAMDDLM-Z (SEQ ID NO: 4124); wherein X— is H or acetyl; and Z is OH or NH2.

In some embodiments, the metal-binding compound of the present invention is selected from the group consisting of: X-NVRVSNGS*P-C(Sx)-LER[Nle]D-Z (SEQ ID NO: 4125), X-PAADA-C(Sx)-[Nle]S*PEEELDG-Z (SEQ ID NO: 4126), X-PAADAI[Nle]S*P-C(Sx)-EELDG-Z (SEQ ID NO: 4127), X-PAKTTKKRPQRATS*N-C(Sx)-FS-Z (SEQ ID NO: 4128), X-PAKTTKKRPQRATSN-C(Sx)-FS*-Z (SEQ ID NO: 4129), X-PANPLMA-C(Sx)-GT*LTRRHQNGRF-Z (SEQ ID NO: 4130), X-P-C(Sx)-DS*LRRKAK-Z (SEQ ID NO: 4131), X-P-C(Sx)-ES*QEAFADLWKK-Z (SEQ ID NO: 4132), X-P-C(Sx)-LS*PGPF-Z (SEQ ID NO: 4133), X-PDTS*Y-C(Sx)-LTPHTEEKY-Z (SEQ ID NO: 4134), X-PDTSY-C(Sx)-LT*PHTEEKY-Z (SEQ ID NO: 1149), X-PDTSYVLT*P-C(Sx)-TEEKY-Z (SEQ ID NO: 1148), X-PDVE[Nle]PS*P-C(Sx)-APSGD-Z (SEQ ID NO: 4135), X-PKRPQRAT*S-C(Sx)-VFAMF-Z (SEQ ID NO: 4136), X-PLKTGEQT*P-C(Sx)-HEHI-Z (SEQ ID NO: 1116), X-PLLR-C(Sx)-SS*RRIRR-Z (SEQ ID NO: 4137), X-PLPWWR-C(Sx)-YT*WVVERDVNTKQR-Z (SEQ ID NO: 4138), X-PLQRQPSS*P-C(Sx)-PTPRN-Z (SEQ ID NO: 1167), X-PLQRQPSSP-C(Sx)-PT*PRN-Z (SEQ ID NO: 4139), X-PLST*W-C(Sx)-GSPPYAAPEAKKK-Z (SEQ ID NO: 4140), X-PLSTW-C(Sx)-GS*PPYAAPEAKKK-Z (SEQ ID NO: 4141), X-PNTED-C(Sx)-ES*PSVKR[Nle]RKKK-Z (SEQ ID NO: 4142), X-PNTEDRES*P-C(Sx)-VKR[Nle]R-Z (SEQ ID NO: 4143), X-PPYNRAVS*L-C(Sx)-SPVSV-Z (SEQ ID NO: 1169), X-PQREGFGRQS*M-C(Sx)-EKR-Z (SEQ ID NO: 4144), X-PR-C(Sx)-KS*LVGTPYW[Nle]APELISR-Z (SEQ ID NO: 4145), X-PR-C(Sx)-KS*LVGTPYW[Nle]APE-Z (SEQ ID NO: 4146), X-PR-C(Sx)-KS*LVGTPYWMAPE-Z (SEQ ID NO: 4147), X-PR-C(Sx)-KS*LVGTPYWM-Z (SEQ ID NO: 4148), X-PR-C(Sx)-KT*PEIFRAL-Z (SEQ ID NO: 4149), X-PRPKT*P-C(Sx)-IFRAL-Z (SEQ ID NO: 4150), X-PRR-C(Sx)-AS*FRRR-Z (SEQ ID NO: 4151), X-PRTSPI[Nle]S*P-C(Sx)-TSLAE-Z (SEQ ID NO: 4152), X-PRTSPI[Nle]SP-C(Sx)-TS*LAE-Z (SEQ ID NO: 4153), X-PS*Y-C(Sx)-RSRSRSRSRSRSRSRSN-Z (SEQ ID NO: 4154), X-PSRPP-C(Sx)-PT*PRRPASV-Z (SEQ ID NO: 4155), X-PSVEP-C(Sx)-LS*QETFSDL-Z (SEQ ID NO: 4156), X-PSWHLADS*P-C(Sx)-VNGAT-Z (SEQ ID NO: 1105), X-PSY-C(Sx)-RS*RSRSRSRSRSRSRSN-Z (SEQ ID NO: 4157), X-PSYGR-C(Sx)-RS*RSRSRSRSRSRSN-Z (SEQ ID NO: 4158), X-PSYGRS*R-C(Sx)-RSRSRSRSRSRSN-Z (SEQ ID NO: 4159), X-PSYGRSR-C(Sx)-RS*RSRSRSRSRSN-Z (SEQ ID NO: 4160), X-PSYGRSRSRS*R-C(Sx)-RSRSRSRSN-Z (SEQ ID NO: 4161), X-PSYGRSRSRSR-C(Sx)-RS*RSRSRSN-Z (SEQ ID NO: 4162), X-PSYGRSRSRSRS*R-C(Sx)-RSRSRSN-Z (SEQ ID NO: 4163), X-PSYGRSRSRSRSR-C(Sx)-RS*RSRSN-Z (SEQ ID NO: 4164), X-PSYGRSRSRSRSRS*R-C(Sx)-RSRSN-Z (SEQ ID NO: 4165), X-PSYGRSRSRSRSRSR-C(Sx)-RS*RSN-Z (SEQ ID NO: 4166), X-PSYGRSRSRSRSRSRS*R-C(Sx)-RSN-Z (SEQ ID NO: 4167), X-PSYGRSRSRSRSRSRSR-C(Sx)-RS*N-Z (SEQ ID NO: 4168), X-PSYGRSRSRSRSRSRSRS*R-C(Sx)-N-Z (SEQ ID NO: 4169), X-PSYGRSRSRSRSRSRSRSRS*N-C(Sx)-Z (SEQ ID NO: 4170), X-PTIPGVTS*P-C(Sx)-SDEPP-Z (SEQ ID NO: 1068), X-PTSPRRYS*P-C(Sx)-AKDLL-Z (SEQ ID NO: 1106), X-PVPEYI-C(Sx)-QS*V-Z (SEQ ID NO: 4171), X-PVVS*G-C(Sx)-TSPRHLSNV-Z (SEQ ID NO: 4172), X-PVVSG-C(Sx)-TS*PRHLSNV-Z (SEQ ID NO: 1067), X-PVVSGDTS*P-C(Sx)-HLSNV-Z (SEQ ID NO: 1066), X-Q[Nle]AGT-C(Sx)-IT*PLKDGFTKKK-Z (SEQ ID NO: 4173), X-QALDNPEYH-C(Sx)-AS*NGP-Z (SEQ ID NO: 4174), X-QANPLMA-C(Sx)-GT*LTRRHQNGRF-Z (SEQ ID NO: 4175), X-QARAK-C(Sx)-QT*PPVSPAP-Z (SEQ ID NO: 1074), X-QARAKTQT*P-C(Sx)-VSPAP-Z (SEQ ID NO: 1073), X-QARAKTQTP-C(Sx)-VS*PAP-Z (SEQ ID NO: 4176), X-Q-C(Sx)-DS*LRRKAK-Z (SEQ ID NO: 4177), X-Q-C(Sx)-LS*PGPF-Z (SEQ ID NO: 4178), X-QE-C(Sx)-FS*DLWKLLP-Z (SEQ ID NO: 1002), X-QETFS*D-C(Sx)-WKLLP-Z (SEQ ID NO: 1001), X-QGV-C(Sx)-ES*PFTRTGEGLDIR-Z (SEQ ID NO: 4179), X-QGVPES*P-C(Sx)-TRTGEGLDIR-Z (SEQ ID NO: 4180), X-QGVPES-C(Sx)-FT*RTGEGLDIR-Z (SEQ ID NO: 4181), X-QGVPESPF-C(Sx)-RT*GEGLDIR-Z (SEQ ID NO: 4182), X-QGVPESPFT*R-C(Sx)-GEGLDIR-Z (SEQ ID NO: 4183), X-QGVPESPFTRT*G-C(Sx)-GLDIR-Z (SEQ ID NO: 4184), X-QHSSPAVS*D-C(Sx)-LPSNS-Z (SEQ ID NO: 1178), X-QKRPQRAT*S-C(Sx)-VFAMF-Z (SEQ ID NO: 4185), X-QLLR-C(Sx)-SS*RRIRR-Z (SEQ ID NO: 4186), X-QLPWWR-C(Sx)-YT*WVVERDVNTKQR-Z (SEQ ID NO: 4187), X-QLS*K-C(Sx)-PGSPTSRSSD-Z (SEQ ID NO: 4188), X-QMFHLDPS*L-C(Sx)-HTIFN-Z (SEQ ID NO: 4189), X-QMFHLDPSL-C(Sx)-HT*IFN-Z (SEQ ID NO: 4190), X-QP-C(Sx)-AS*PVV-Z (SEQ ID NO: 4191), X-QQREGFGRQS*M-C(Sx)-EKR-Z (SEQ ID NO: 4192), and X-QRE-C(Sx)-VS*SLDSSSLSLKKK-Z (SEQ ID NO: 4193); wherein X— is H or acetyl; and Z is OH or NH2.

In some embodiments, the metal-binding compound of the present invention is selected from the group consisting of: X-QRR-C(Sx)-AS*FRRR-Z (SEQ ID NO: 4194), X-QS[Nle]VV-C(Sx)-QT*PLHTARV-Z (SEQ ID NO: 4195), X-QYHFVAS-C(Sx)-ST*IERDRQRPYS-Z (SEQ ID NO: 1198), X-QYHFVASSST*I-C(Sx)-RDRQRPYS-Z (SEQ ID NO: 1197), X-R[Nle]PWWR-C(Sx)-YT*WVVERDVNTKQR-Z (SEQ ID NO: 4196), X-R[Nle]R-C(Sx)-AS*FRRR-Z (SEQ ID NO: 4197), X-RADPQ-C(Sx)-PS*RTPVQGP-Z (SEQ ID NO: 1190), X-RAKTTKKRPQRATS*N-C(Sx)-FS-Z (SEQ ID NO: 4198), X-RAKITKKRPQRATSN-C(Sx)-FS*-Z (SEQ ID NO: 4199), X-RANPLMA-C(Sx)-GT*LTRRHQNGRF-Z (SEQ ID NO: 4200), X-RAPWWR-C(Sx)-YT*WVVERDVNTKQR-Z (SEQ ID NO: 4201), X-RAR-C(Sx)-AS*FRRR-Z (SEQ ID NO: 4202), X-RAR-C(Sx)-LS*FAEG-Z (SEQ ID NO: 4203), X-RAR-C(Sx)-LS*FAEPG-Z (SEQ ID NO: 4204), X-RARR-C(Sx)-LS*FSGFGSR-Z (SEQ ID NO: 4205), X-R-C(Sx)-AS*FRRR-Z (SEQ ID NO: 4206), X-R-C(Sx)-AS*FRR-Z (SEQ ID NO: 4207), X-R-C(Sx)-AS*FR-Z (SEQ ID NO: 4208), X-R-C(Sx)-DS*LRRKAK-Z (SEQ ID NO: 4209), X-R-C(Sx)-FS*LRRKAK-Z (SEQ ID NO: 1191), X-R-C(Sx)-LS*FAEG-Z (SEQ ID NO: 4210), X-R-C(Sx)-LS*FAEPG-Z (SEQ ID NO: 4211), X-R-C(Sx)-LS*PGPF-Z (SEQ ID NO: 4212), X-RDPWWR-C(Sx)-YT*WVVERDVNTKQR-Z (SEQ ID NO: 4213), X-RDR-C(Sx)-AS*FRRR-Z (SEQ ID NO: 4214), X-RE-C(Sx)-LS*RRP[pS]YR-Z (SEQ ID NO: 4215), X-REPWWR-C(Sx)-YT*WVVERDVNTKQR-Z (SEQ ID NO: 4216), X-RER-C(Sx)-AS*FRRR-Z (SEQ ID NO: 4217), X-RFDS*R-C(Sx)-ISGLLTTAKKK-Z (SEQ ID NO: 4218), X-RFDSR-C(Sx)-IS*GLLTTAKKK-Z (SEQ ID NO: 4219), X-RFI-C(Sx)-PT*ALIRF-Z (SEQ ID NO: 4220), X-RFI-C(Sx)-PT*NRRRF-Z (SEQ ID NO: 4221), X-RFILPT*A-C(Sx)-IRF-Z (SEQ ID NO: 4222), X-RFILPT*N-C(Sx)-RRF-Z (SEQ ID NO: 4223), X-RFPWWR-C(Sx)-YT*WVVERDVNTKQR-Z (SEQ ID NO: 4224), X-RFR-C(Sx)-AS*FRRR-Z (SEQ ID NO: 4225), X-RGGPEPLS*P-C(Sx)-PVSPL-Z (SEQ ID NO: 1088), X-RGPWWR-C(Sx)-YT*WVVERDVNTKQR-Z (SEQ ID NO: 4226), X-RGR-C(Sx)-AS*FRRR-Z (SEQ ID NO: 4227), X-RGS[Nle]AAPS*L-C(Sx)-PSLGP-Z (SEQ ID NO: 4228), X-RGS[Nle]AAPSL-C(Sx)-PS*LGP-Z (SEQ ID NO: 4229), X-RHD[pS]GLDS*M-C(Sx)-DEEYE-Z (SEQ ID NO: 4230), X-RHPWWR-C(Sx)-YT*WVVERDVNTKQR-Z (SEQ ID NO: 4231), X-RHR-C(Sx)-AS*FRRR-Z (SEQ ID NO: 4232), X-RIPWWR-C(Sx)-YT*WVVERDVNTKQR-Z (SEQ ID NO: 4233), X-RIR-C(Sx)-AS*FRRR-Z (SEQ ID NO: 4234), X-RKKYKFN-C(Sx)-DT*ERRRFL-Z (SEQ ID NO: 4235), X-RKPWWR-C(Sx)-YT*WVVERDVNTKQR-Z (SEQ ID NO: 4236), X-RKR-C(Sx)-AS*FRRR-Z (SEQ ID NO: 4237), X-RKRKGS*F-C(Sx)-YGG-Z (SEQ ID NO: 4238), X-RKRPQRAT*S-C(Sx)-VFAMF-Z (SEQ ID NO: 4239), and X-RLGWWR-C(Sx)-FT*LRRARQGNTKQR-Z (SEQ ID NO: 4240); wherein X— is H or acetyl; and Z is OH or NH2.

In some embodiments, the metal-binding compound of the present invention is selected from the group consisting of: X-RLGWWR-C(Sx)-YT*LQRARDGNTKQR-Z (SEQ ID NO: 4241), X-RLGWWR-C(Sx)-YT*LQRARDVNTKQR-Z (SEQ ID NO: 4242), X-RLGWWR-C(Sx)-YT*LQRAREGNTKQR-Z (SEQ ID NO: 4243), X-RLGWWR-C(Sx)-YT*LQRAREVNTKQR-Z (SEQ ID NO: 4244), X-RLGWWR-C(Sx)-YT*LQRARQGNTKQR-Z (SEQ ID NO: 4245), X-RLGWWR-C(Sx)-YT*LQRARQVNTKQR-Z (SEQ ID NO: 4246), X-RLGWWR-C(Sx)-YT*LQRERDGNTKQR-Z (SEQ ID NO: 4247), X-RLGWWR-C(Sx)-YT*LQRERDVNTKQR-Z (SEQ ID NO: 4248), X-RLGWWR-C(Sx)-YT*LQREREGNTKQR-Z (SEQ ID NO: 4249), X-RLGWWR-C(Sx)-YT*LQREREVNTKQR-Z (SEQ ID NO: 4250), X-RLGWWR-C(Sx)-YT*LQRERQGNTKQR-Z (SEQ ID NO: 4251), X-RLGWWR-C(Sx)-YT*LQRERQVNTKQR-Z (SEQ ID NO: 4252), X-RLGWWR-C(Sx)-YT*LQVARDGNTKQR-Z (SEQ ID NO: 4253), X-RLGWWR-C(Sx)-YT*LQVARDVNTKQR-Z (SEQ ID NO: 4254), X-RLGWWR-C(Sx)-YT*LQVAREGNTKQR-Z (SEQ ID NO: 4255), X-RLGWWR-C(Sx)-YT*LQVAREVNTKQR-Z (SEQ ID NO: 4256), X-RLGWWR-C(Sx)-YT*LQVARQGNTKQR-Z (SEQ ID NO: 4257), X-RLGWWR-C(Sx)-YT*LQVARQVNTKQR-Z (SEQ ID NO: 4258), X-RLGWWR-C(Sx)-YT*LQVERDGNTKQR-Z (SEQ ID NO: 4259), X-RLGWWR-C(Sx)-YT*LQVERDVNTKQR-Z (SEQ ID NO: 4260), X-RLGWWR-C(Sx)-YT*LQVEREGNTKQR-Z (SEQ ID NO: 4261), X-RLGWWR-C(Sx)-YT*LQVEREVNTKQR-Z (SEQ ID NO: 4262), X-RLGWWR-C(Sx)-YT*LQVERQGNTKQR-Z (SEQ ID NO: 4263), X-RLGWWR-C(Sx)-YT*LQVERQVNTKQR-Z (SEQ ID NO: 4264), X-RLGWWR-C(Sx)-YT*LRRARDGNTKQR-Z (SEQ ID NO: 4265), X-RLGWWR-C(Sx)-YT*LRRARDVNTKQR-Z (SEQ ID NO: 4266), X-RLGWWR-C(Sx)-YT*LRRAREGNTKQR-Z (SEQ ID NO: 4267), X-RLGWWR-C(Sx)-YT*LRRAREVNTKQR-Z (SEQ ID NO: 4268), X-RLGWWR-C(Sx)-YT*LRRARQGNTKQR-Z (SEQ ID NO: 4269), X-RLGWWR-C(Sx)-YT*LRRARQVNTKQR-Z (SEQ ID NO: 4270), X-RLGWWR-C(Sx)-YT*LRRERDGNTKQR-Z (SEQ ID NO: 4271), X-RLGWWR-C(Sx)-YT*LRRERDVNTKQR-Z (SEQ ID NO: 4272), X-RLGWWR-C(Sx)-YT*LRREREGNTKQR-Z (SEQ ID NO: 4273), X-RLGWWR-C(Sx)-YT*LRREREVNTKQR-Z (SEQ ID NO: 4274), X-RLGWWR-C(Sx)-YT*LRRERQGNTKQR-Z (SEQ ID NO: 4275), X-RLGWWR-C(Sx)-YT*LRRERQVNTKQR-Z (SEQ ID NO: 4276), X-RLGWWR-C(Sx)-YT*LRVARDGNTKQR-Z (SEQ ID NO: 4277), X-RLGWWR-C(Sx)-YT*LRVARDVNTKQR-Z (SEQ ID NO: 4278), X-RLGWWR-C(Sx)-YT*LRVAREGNTKQR-Z (SEQ ID NO: 4279), X-RLGWWR-C(Sx)-YT*LRVAREVNTKQR-Z (SEQ ID NO: 4280), X-RLGWWR-C(Sx)-YT*LRVARQGNTKQR-Z (SEQ ID NO: 4281), X-RLGWWR-C(Sx)-YT*LRVARQVNTKQR-Z (SEQ ID NO: 4282), X-RLGWWR-C(Sx)-YT*LRVERDGNTKQR-Z (SEQ ID NO: 4283), X-RLGWWR-C(Sx)-YT*LRVERDVNTKQR-Z (SEQ ID NO: 4284), X-RLGWWR-C(Sx)-YT*LRVEREGNTKQR-Z (SEQ ID NO: 4285), X-RLGWWR-C(Sx)-YT*LRVEREVNTKQR-Z (SEQ ID NO: 4286), X-RLGWWR-C(Sx)-YT*LRVERQGNTKQR-Z (SEQ ID NO: 4287), X-RLGWWR-C(Sx)-YT*LRVERQVNTKQR-Z (SEQ ID NO: 4288), X-RLGWWR-C(Sx)-YT*LVRARDGNTKQR-Z (SEQ ID NO: 4289), X-RLGWWR-C(Sx)-YT*LVRARDVNTKQR-Z (SEQ ID NO: 4290), and X-RLGWWR-C(Sx)-YT*LVRAREGNTKQR-Z (SEQ ID NO: 4291); wherein X— is H or acetyl; and Z is OH or NH2.

In some embodiments, the metal-binding compound of the present invention is selected from the group consisting of: X-RLGWWR-C(Sx)-YT*LVRAREVNTKQR-Z (SEQ ID NO: 4292), X-RLGWWR-C(Sx)-YT*LVRARQGNTKQR-Z (SEQ ID NO: 4293), X-RLGWWR-C(Sx)-YT*LVRARQVNTKQR-Z (SEQ ID NO: 4294), X-RLGWWR-C(Sx)-YT*LVRERDGNTKQR-Z (SEQ ID NO: 4295), X-RLGWWR-C(Sx)-YT*LVRERDVNTKQR-Z (SEQ ID NO: 4296), X-RLGWWR-C(Sx)-YT*LVREREGNTKQR-Z (SEQ ID NO: 4297), X-RLGWWR-C(Sx)-YT*LVREREVNTKQR-Z (SEQ ID NO: 4298), X-RLGWWR-C(Sx)-YT*LVRERQGNTKQR-Z (SEQ ID NO: 4299), X-RLGWWR-C(Sx)-YT*LVRERQVNTKQR-Z (SEQ ID NO: 4300), X-RLGWWR-C(Sx)-YT*LVVARDGNTKQR-Z (SEQ ID NO: 4301), X-RLGWWR-C(Sx)-YT*LVVARDVNTKQR-Z (SEQ ID NO: 4302), X-RLGWWR-C(Sx)-YT*LVVAREGNTKQR-Z (SEQ ID NO: 4303), X-RLGWWR-C(Sx)-YT*LVVAREVNTKQR-Z (SEQ ID NO: 4304), X-RLGWWR-C(Sx)-YT*LVVARQGNTKQR-Z (SEQ ID NO: 4305), X-RLGWWR-C(Sx)-YT*LVVARQVNTKQR-Z (SEQ ID NO: 4306), X-RLGWWR-C(Sx)-YT*LVVERDGNTKQR-Z (SEQ ID NO: 4307), X-RLGWWR-C(Sx)-YT*LVVERDVNTKQR-Z (SEQ ID NO: 4308), X-RLGWWR-C(Sx)-YT*LVVEREGNTKQR-Z (SEQ ID NO: 4309), X-RLGWWR-C(Sx)-YT*LVVEREVNTKQR-Z (SEQ ID NO: 4310), X-RLGWWR-C(Sx)-YT*LVVERQGNTKQR-Z (SEQ ID NO: 4311), X-RLGWWR-C(Sx)-YT*LVVERQVNTKQR-Z (SEQ ID NO: 4312), X-RLGWWR-C(Sx)-YT*WQRARDGNTKQR-Z (SEQ ID NO: 4313), X-RLGWWR-C(Sx)-YT*WQRARDVNTKQR-Z (SEQ ID NO: 4314), X-RLGWWR-C(Sx)-YT*WQRAREGNTKQR-Z (SEQ ID NO: 4315), X-RLGWWR-C(Sx)-YT*WQRAREVNTKQR-Z (SEQ ID NO: 4316), X-RLGWWR-C(Sx)-YT*WQRARQGNTKQR-Z (SEQ ID NO: 4317), X-RLGWWR-C(Sx)-YT*WQRARQVNTKQR-Z (SEQ ID NO: 4318), X-RLGWWR-C(Sx)-YT*WQRERDGNTKQR-Z (SEQ ID NO: 4319), X-RLGWWR-C(Sx)-YT*WQRERDVNTKQR-Z (SEQ ID NO: 4320), X-RLGWWR-C(Sx)-YT*WQREREGNTKQR-Z (SEQ ID NO: 4321), X-RLGWWR-C(Sx)-YT*WQREREVNTKQR-Z (SEQ ID NO: 4322), X-RLGWWR-C(Sx)-YT*WQRERQGNTKQR-Z (SEQ ID NO: 4323), X-RLGWWR-C(Sx)-YT*WQRERQVNTKQR-Z (SEQ ID NO: 4324), X-RLGWWR-C(Sx)-YT*WQVARDGNTKQR-Z (SEQ ID NO: 4325), X-RLGWWR-C(Sx)-YT*WQVARDVNTKQR-Z (SEQ ID NO: 4326), X-RLGWWR-C(Sx)-YT*WQVAREGNTKQR-Z (SEQ ID NO: 4327), X-RLGWWR-C(Sx)-YT*WQVAREVNTKQR-Z (SEQ ID NO: 4328), X-RLGWWR-C(Sx)-YT*WQVARQGNTKQR-Z (SEQ ID NO: 4329), X-RLGWWR-C(Sx)-YT*WQVARQVNTKQR-Z (SEQ ID NO: 4330), X-RLGWWR-C(Sx)-YT*WQVERDGNTKQR-Z (SEQ ID NO: 4331), X-RLGWWR-C(Sx)-YT*WQVERDVNTKQR-Z (SEQ ID NO: 4332), X-RLGWWR-C(Sx)-YT*WQVEREGNTKQR-Z (SEQ ID NO: 4333), X-RLGWWR-C(Sx)-YT*WQVEREVNTKQR-Z (SEQ ID NO: 4334), X-RLGWWR-C(Sx)-YT*WQVERQGNTKQR-Z (SEQ ID NO: 4335), X-RLGWWR-C(Sx)-YT*WQVERQVNTKQR-Z (SEQ ID NO: 4336), X-RLGWWR-C(Sx)-YT*WRRARDGNTKQR-Z (SEQ ID NO: 4337), X-RLGWWR-C(Sx)-YT*WRRARDVNTKQR-Z (SEQ ID NO: 4338), X-RLGWWR-C(Sx)-YT*WRRAREGNTKQR-Z (SEQ ID NO: 4339), X-RLGWWR-C(Sx)-YT*WRRAREVNTKQR-Z (SEQ ID NO: 4340), X-RLGWWR-C(Sx)-YT*WRRARQGNTKQR-Z (SEQ ID NO: 4341), X-RLGWWR-C(Sx)-YT*WRRARQVNTKQR-Z (SEQ ID NO: 4342), X-RLGWWR-C(Sx)-YT*WRRERDGNTKQR-Z (SEQ ID NO: 4343), X-RLGWWR-C(Sx)-YT*WRRERDVNTKQR-Z (SEQ ID NO: 4344), X-RLGWWR-C(Sx)-YT*WRREREGNTKQR-Z (SEQ ID NO: 4345), X-RLGWWR-C(Sx)-YT*WRREREVNTKQR-Z (SEQ ID NO: 4346), X-RLGWWR-C(Sx)-YT*WRRERQGNTKQR-Z (SEQ ID NO: 4347), X-RLGWWR-C(Sx)-YT*WRRERQVNTKQR-Z (SEQ ID NO: 4348), X-RLGWWR-C(Sx)-YT*WRVARDGNTKQR-Z (SEQ ID NO: 4349), X-RLGWWR-C(Sx)-YT*WRVARDVNTKQR-Z (SEQ ID NO: 4350), X-RLGWWR-C(Sx)-YT*WRVAREGNTKQR-Z (SEQ ID NO: 4351), X-RLGWWR-C(Sx)-YT*WRVAREVNTKQR-Z (SEQ ID NO: 4352), X-RLGWWR-C(Sx)-YT*WRVARQGNTKQR-Z (SEQ ID NO: 4353), X-RLGWWR-C(Sx)-YT*WRVARQVNTKQR-Z (SEQ ID NO: 4354), X-RLGWWR-C(Sx)-YT*WRVERDGNTKQR-Z (SEQ ID NO: 4355), X-RLGWWR-C(Sx)-YT*WRVERDVNTKQR-Z (SEQ ID NO: 4356), X-RLGWWR-C(Sx)-YT*WRVEREGNTKQR-Z (SEQ ID NO: 4357), X-RLGWWR-C(Sx)-YT*WRVEREVNTKQR-Z (SEQ ID NO: 4358), X-RLGWWR-C(Sx)-YT*WRVERQGNTKQR-Z (SEQ ID NO: 4359), and X-RLGWWR-C(Sx)-YT*WRVERQVNTKQR-Z (SEQ ID NO: 4360); wherein X— is H or acetyl; and Z is OH or NH2.

In some embodiments, the metal-binding compound of the present invention is selected from the group consisting of: X-RLGWWR-C(Sx)-YT*WVRARDGNTKQR-Z (SEQ ID NO: 4361), X-RLGWWR-C(Sx)-YT*WVRARDVNTKQR-Z (SEQ ID NO: 4362), X-RLGWWR-C(Sx)-YT*WVRAREGNTKQR-Z (SEQ ID NO: 4363), X-RLGWWR-C(Sx)-YT*WVRAREVNTKQR-Z (SEQ ID NO: 4364), X-RLGWWR-C(Sx)-YT*WVRARQGNTKQR-Z (SEQ ID NO: 4365), X-RLGWWR-C(Sx)-YT*WVRARQVNTKQR-Z (SEQ ID NO: 4366), X-RLGWWR-C(Sx)-YT*WVRERDGNTKQR-Z (SEQ ID NO: 4367), X-RLGWWR-C(Sx)-YT*WVRERDVNTKQR-Z (SEQ ID NO: 4368), X-RLGWWR-C(Sx)-YT*WVREREGNTKQR-Z (SEQ ID NO: 4369), X-RLGWWR-C(Sx)-YT*WVREREVNTKQR-Z (SEQ ID NO: 4370), X-RLGWWR-C(Sx)-YT*WVRERQGNTKQR-Z (SEQ ID NO: 4371), X-RLGWWR-C(Sx)-YT*WVRERQVNTKQR-Z (SEQ ID NO: 4372), X-RLGWWR-C(Sx)-YT*WVVARDGNTKQR-Z (SEQ ID NO: 4373), X-RLGWWR-C(Sx)-YT*WVVARDVNTKQR-Z (SEQ ID NO: 4374), X-RLGWWR-C(Sx)-YT*WVVAREGNTKQR-Z (SEQ ID NO: 4375), X-RLGWWR-C(Sx)-YT*WVVAREVNTKQR-Z (SEQ ID NO: 4376), X-RLGWWR-C(Sx)-YT*WVVARQGNTKQR-Z (SEQ ID NO: 4377), X-RLGWWR-C(Sx)-YT*WVVARQVNTKQR-Z (SEQ ID NO: 4378), X-RLGWWR-C(Sx)-YT*WVVERDGNTKQR-Z (SEQ ID NO: 4379), X-RLGWWR-C(Sx)-YT*WVVERDVNTKQR-Z (SEQ ID NO: 4380), X-RLGWWR-C(Sx)-YT*WVVEREGNTKQR-Z (SEQ ID NO: 4381), X-RLGWWR-C(Sx)-YT*WVVEREVNTKQR-Z (SEQ ID NO: 4382), X-RLGWWR-C(Sx)-YT*WVVERQGNTKQR-Z (SEQ ID NO: 4383), X-RLGWWR-C(Sx)-YT*WVVERQVNTKQR-Z (SEQ ID NO: 4384), X-RLLR-C(Sx)-SS*RRIRR-Z (SEQ ID NO: 4385), X-RLPWWR-C(Sx)-FT*WVRERDVNTKQR-Z (SEQ ID NO: 4386), X-RLPWWR-C(Sx)-FT*WVVERDVNTKQR-Z (SEQ ID NO: 4387), X-RLPWWR-C(Sx)-YT*LQRARDGNTKQR-Z (SEQ ID NO: 4388), X-RLPWWR-C(Sx)-YT*LQRARDVNTKQR-Z (SEQ ID NO: 4389), X-RLPWWR-C(Sx)-YT*LQRAREGNTKQR-Z (SEQ ID NO: 4390), and X-RLPWWR-C(Sx)-YT*LQRAREVNTKQR-Z (SEQ ID NO: 4391); wherein X is H or acetyl; and Z is OH or NH2.

In some embodiments, the metal-binding compound of the present invention is selected from the group consisting of: X-RLPWWR-C(Sx)-YT*LQRARQGNTKQR-Z (SEQ ID NO: 4392), X-RLPWWR-C(Sx)-YT*LQRARQVNTKQR-Z (SEQ ID NO: 4393), X-RLPWWR-C(Sx)-YT*LQRERDGNTKQR-Z (SEQ ID NO: 4394), X-RLPWWR-C(Sx)-YT*LQRERDVNTKQR-Z (SEQ ID NO: 4395), X-RLPWWR-C(Sx)-YT*LQREREGNTKQR-Z (SEQ ID NO: 4396), X-RLPWWR-C(Sx)-YT*LQREREVNTKQR-Z (SEQ ID NO: 4397), X-RLPWWR-C(Sx)-YT*LQRERQGNTKQR-Z (SEQ ID NO: 4398), X-RLPWWR-C(Sx)-YT*LQRERQVNTKQR-Z (SEQ ID NO: 4399), X-RLPWWR-C(Sx)-YT*LQVARDGNTKQR-Z (SEQ ID NO: 4400), X-RLPWWR-C(Sx)-YT*LQVARDVNTKQR-Z (SEQ ID NO: 4401), X-RLPWWR-C(Sx)-YT*LQVAREGNTKQR-Z (SEQ ID NO: 4402), X-RLPWWR-C(Sx)-YT*LQVAREVNTKQR-Z (SEQ ID NO: 4403), X-RLPWWR-C(Sx)-YT*LQVARQGNTKQR-Z (SEQ ID NO: 4404), X-RLPWWR-C(Sx)-YT*LQVARQVNTKQR-Z (SEQ ID NO: 4405), X-RLPWWR-C(Sx)-YT*LQVERDGNTKQR-Z (SEQ ID NO: 4406), X-RLPWWR-C(Sx)-YT*LQVERDVNTKQR-Z (SEQ ID NO: 4407), X-RLPWWR-C(Sx)-YT*LQVEREGNTKQR-Z (SEQ ID NO: 4408), X-RLPWWR-C(Sx)-YT*LQVEREVNTKQR-Z (SEQ ID NO: 4409), X-RLPWWR-C(Sx)-YT*LQVERQGNTKQR-Z (SEQ ID NO: 4410), X-RLPWWR-C(Sx)-YT*LQVERQVNTKQR-Z (SEQ ID NO: 4411), X-RLPWWR-C(Sx)-YT*LRRARDGNTKQR-Z (SEQ ID NO: 4412), X-RLPWWR-C(Sx)-YT*LRRARDVNTKQR-Z (SEQ ID NO: 4413), X-RLPWWR-C(Sx)-YT*LRRAREGNTKQR-Z (SEQ ID NO: 4414), X-RLPWWR-C(Sx)-YT*LRRAREVNTKQR-Z (SEQ ID NO: 4415), X-RLPWWR-C(Sx)-YT*LRRARQGNTKQR-Z (SEQ ID NO: 4416), X-RLPWWR-C(Sx)-YT*LRRARQVNTKQR-Z (SEQ ID NO: 4417), X-RLPWWR-C(Sx)-YT*LRRERDGNTKQR-Z (SEQ ID NO: 4418), X-RLPWWR-C(Sx)-YT*LRRERDVNTKQR-Z (SEQ ID NO: 4419), X-RLPWWR-C(Sx)-YT*LRREREGNTKQR-Z (SEQ ID NO: 4420), X-RLPWWR-C(Sx)-YT*LRREREVNTKQR-Z (SEQ ID NO: 4421), X-RLPWWR-C(Sx)-YT*LRRERQGNTKQR-Z (SEQ ID NO: 4422), X-RLPWWR-C(Sx)-YT*LRRERQVNTKQR-Z (SEQ ID NO: 4423), X-RLPWWR-C(Sx)-YT*LRVARDGNTKQR-Z (SEQ ID NO: 4424), X-RLPWWR-C(Sx)-YT*LRVARDVNTKQR-Z (SEQ ID NO: 4425), and X-RLPWWR-C(Sx)-YT*LRVAREGNTKQR-Z (SEQ ID NO: 4426); wherein X— is H or acetyl; and Z is OH or NH2.

In some embodiments, the metal-binding compound of the present invention is selected from the group consisting of: X-RLPWWR-C(Sx)-YT*LRVAREVNTKQR-Z (SEQ ID NO: 4427), X-RLPWWR-C(Sx)-YT*LRVARQGNTKQR-Z (SEQ ID NO: 4428), X-RLPWWR-C(Sx)-YT*LRVARQVNTKQR-Z (SEQ ID NO: 4429), X-RLPWWR-C(Sx)-YT*LRVERDGNTKQR-Z (SEQ ID NO: 4430), X-RLPWWR-C(Sx)-YT*LRVERDVNTKQR-Z (SEQ ID NO: 4431), X-RLPWWR-C(Sx)-YT*LRVEREGNTKQR-Z (SEQ ID NO: 4432), X-RLPWWR-C(Sx)-YT*LRVEREVNTKQR-Z (SEQ ID NO: 4433), X-RLPWWR-C(Sx)-YT*LRVERQGNTKQR-Z (SEQ ID NO: 4434), X-RLPWWR-C(Sx)-YT*LRVERQVNTKQR-Z (SEQ ID NO: 4435), X-RLPWWR-C(Sx)-YT*LVRARDGNTKQR-Z (SEQ ID NO: 4436), X-RLPWWR-C(Sx)-YT*LVRARDVNTKQR-Z (SEQ ID NO: 4437), X-RLPWWR-C(Sx)-YT*LVRAREGNTKQR-Z (SEQ ID NO: 4438), X-RLPWWR-C(Sx)-YT*LVRAREVNTKQR-Z (SEQ ID NO: 4439), X-RLPWWR-C(Sx)-YT*LVRARQGNTKQR-Z (SEQ ID NO: 4440), X-RLPWWR-C(Sx)-YT*LVRARQVNTKQR-Z (SEQ ID NO: 4441), X-RLPWWR-C(Sx)-YT*LVRERDGNTKQR-Z (SEQ ID NO: 4442), X-RLPWWR-C(Sx)-YT*LVRERDVNTKQR-Z (SEQ ID NO: 4443), X-RLPWWR-C(Sx)-YT*LVREREGNTKQR-Z (SEQ ID NO: 4444), X-RLPWWR-C(Sx)-YT*LVREREVNTKQR-Z (SEQ ID NO: 4445), X-RLPWWR-C(Sx)-YT*LVRERQGNTKQR-Z (SEQ ID NO: 4446), X-RLPWWR-C(Sx)-YT*LVRERQVNTKQR-Z (SEQ ID NO: 4447), X-RLPWWR-C(Sx)-YT*LVVARDGNTKQR-Z (SEQ ID NO: 4448), X-RLPWWR-C(Sx)-YT*LVVARDVNTKQR-Z (SEQ ID NO: 4449), X-RLPWWR-C(Sx)-YT*LVVAREGNTKQR-Z (SEQ ID NO: 4450), X-RLPWWR-C(Sx)-YT*LVVAREVNTKQR-Z (SEQ ID NO: 4451), X-RLPWWR-C(Sx)-YT*LVVARQGNTKQR-Z (SEQ ID NO: 4452), X-RLPWWR-C(Sx)-YT*LVVARQVNTKQR-Z (SEQ ID NO: 4453), X-RLPWWR-C(Sx)-YT*LVVERDGNTKQR-Z (SEQ ID NO: 4454), X-RLPWWR-C(Sx)-YT*LVVERDVNTKQR-Z (SEQ ID NO: 4455), X-RLPWWR-C(Sx)-YT*LVVEREGNTKQR-Z (SEQ ID NO: 4456), X-RLPWWR-C(Sx)-YT*LVVEREVNTKQR-Z (SEQ ID NO: 4457), X-RLPWWR-C(Sx)-YT*LVVERQGNTKQR-Z (SEQ ID NO: 4458), X-RLPWWR-C(Sx)-YT*LVVERQVNTKQR-Z (SEQ ID NO: 4459), X-RLPWWR-C(Sx)-YT*WQRARDGNTKQR-Z (SEQ ID NO: 4460), X-RLPWWR-C(Sx)-YT*WQRARDVNTKQR-Z (SEQ ID NO: 4461), X-RLPWWR-C(Sx)-YT*WQRAREGNTKQR-Z (SEQ ID NO: 4462), X-RLPWWR-C(Sx)-YT*WQRAREVNTKQR-Z (SEQ ID NO: 4463), X-RLPWWR-C(Sx)-YT*WQRARQGNTKQR-Z (SEQ ID NO: 4464), X-RLPWWR-C(Sx)-YT*WQRARQVNTKQR-Z (SEQ ID NO: 4465), X-RLPWWR-C(Sx)-YT*WQRERDGNTKQR-Z (SEQ ID NO: 4466), X-RLPWWR-C(Sx)-YT*WQRERDVNTKQR-Z (SEQ ID NO: 4467), X-RLPWWR-C(Sx)-YT*WQREREGNTKQR-Z (SEQ ID NO: 4468), X-RLPWWR-C(Sx)-YT*WQREREVNTKQR-Z (SEQ ID NO: 4469), X-RLPWWR-C(Sx)-YT*WQRERQGNTKQR-Z (SEQ ID NO: 4470), X-RLPWWR-C(Sx)-YT*WQRERQVNTKQR-Z (SEQ ID NO: 4471), X-RLPWWR-C(Sx)-YT*WQVARDGNTKQR-Z (SEQ ID NO: 4472), X-RLPWWR-C(Sx)-YT*WQVARDVNTKQR-Z (SEQ ID NO: 4473), X-RLPWWR-C(Sx)-YT*WQVAREGNTKQR-Z (SEQ ID NO: 4474), X-RLPWWR-C(Sx)-YT*WQVAREVNTKQR-Z (SEQ ID NO: 4475), X-RLPWWR-C(Sx)-YT*WQVARQGNTKQR-Z (SEQ ID NO: 4476), X-RLPWWR-C(Sx)-YT*WQVARQVNTKQR-Z (SEQ ID NO: 4477), X-RLPWWR-C(Sx)-YT*WQVERDGNTKQR-Z (SEQ ID NO: 4478), X-RLPWWR-C(Sx)-YT*WQVERDVNTKQR-Z (SEQ ID NO: 4479), X-RLPWWR-C(Sx)-YT*WQVEREGNTKQR-Z (SEQ ID NO: 4480), and X-RLPWWR-C(Sx)-YT*WQVEREVNTKQR-Z (SEQ ID NO: 4481); wherein X is H or acetyl; and Z is OH or NH2.

In some embodiments, the metal-binding compound of the present invention is selected from the group consisting of: X-RLPWWR-C(Sx)-YT*WQVERQGNTKQR-Z (SEQ ID NO: 4482), X-RLPWWR-C(Sx)-YT*WQVERQVNTKQR-Z (SEQ ID NO: 4483), X-RLPWWR-C(Sx)-YT*WRRARDGNTKQR-Z (SEQ ID NO: 4484), X-RLPWWR-C(Sx)-YT*WRRARDVNTKQR-Z (SEQ ID NO: 4485), X-RLPWWR-C(Sx)-YT*WRRAREGNTKQR-Z (SEQ ID NO: 4486), X-RLPWWR-C(Sx)-YT*WRRAREVNTKQR-Z (SEQ ID NO: 4487), X-RLPWWR-C(Sx)-YT*WRRARQGNTKQR-Z (SEQ ID NO: 4488), X-RLPWWR-C(Sx)-YT*WRRARQVNTKQR-Z (SEQ ID NO: 4489), X-RLPWWR-C(Sx)-YT*WRRERDGNTKQR-Z (SEQ ID NO: 4490), X-RLPWWR-C(Sx)-YT*WRRERDVNTKQR-Z (SEQ ID NO: 4491), X-RLPWWR-C(Sx)-YT*WRREREGNTKQR-Z (SEQ ID NO: 4492), X-RLPWWR-C(Sx)-YT*WRREREVNTKQR-Z (SEQ ID NO: 4493), X-RLPWWR-C(Sx)-YT*WRRERQGNTKQR-Z (SEQ ID NO: 4494), X-RLPWWR-C(Sx)-YT*WRRERQVNTKQR-Z (SEQ ID NO: 4495), X-RLPWWR-C(Sx)-YT*WRVARDGNTKQR-Z (SEQ ID NO: 4496), X-RLPWWR-C(Sx)-YT*WRVARDVNTKQR-Z (SEQ ID NO: 4497), X-RLPWWR-C(Sx)-YT*WRVAREGNTKQR-Z (SEQ ID NO: 4498), X-RLPWWR-C(Sx)-YT*WRVAREVNTKQR-Z (SEQ ID NO: 4499), X-RLPWWR-C(Sx)-YT*WRVARQGNTKQR-Z (SEQ ID NO: 4500), X-RLPWWR-C(Sx)-YT*WRVARQVNTKQR-Z (SEQ ID NO: 4501), X-RLPWWR-C(Sx)-YT*WRVERDGNTKQR-Z (SEQ ID NO: 4502), X-RLPWWR-C(Sx)-YT*WRVERDVNTKQR-Z (SEQ ID NO: 4503), X-RLPWWR-C(Sx)-YT*WRVEREGNTKQR-Z (SEQ ID NO: 4504), X-RLPWWR-C(Sx)-YT*WRVEREVNTKQR-Z (SEQ ID NO: 4505), X-RLPWWR-C(Sx)-YT*WRVERQGNTKQR-Z (SEQ ID NO: 4506), X-RLPWWR-C(Sx)-YT*WRVERQVNTKQR-Z (SEQ ID NO: 4507), X-RLPWWR-C(Sx)-YT*WVRARDGNTKQR-Z (SEQ ID NO: 4508), X-RLPWWR-C(Sx)-YT*WVRARDVNTKQR-Z (SEQ ID NO: 4509), X-RLPWWR-C(Sx)-YT*WVRAREGNTKQR-Z (SEQ ID NO: 4510), X-RLPWWR-C(Sx)-YT*WVRAREVNTKQR-Z (SEQ ID NO: 4511), X-RLPWWR-C(Sx)-YT*WVRARQGNTKQR-Z (SEQ ID NO: 4512), X-RLPWWR-C(Sx)-YT*WVRARQVNTKQR-Z (SEQ ID NO: 4513), X-RLPWWR-C(Sx)-YT*WVRERDGNTKQR-Z (SEQ ID NO: 4514), X-RLPWWR-C(Sx)-YT*WVRERDVNTKQR-Z (SEQ ID NO: 4515), X-RLPWWR-C(Sx)-YT*WVREREGNTKQR-Z (SEQ ID NO: 4516), X-RLPWWR-C(Sx)-YT*WVREREVNTKQR-Z (SEQ ID NO: 4517), X-RLPWWR-C(Sx)-YT*WVRERQGNTKQR-Z (SEQ ID NO: 4518), X-RLPWWR-C(Sx)-YT*WVRERQVNTKQR-Z (SEQ ID NO: 4519), X-RLPWWR-C(Sx)-YT*WVVARDGNTKQR-Z (SEQ ID NO: 4520), X-RLPWWR-C(Sx)-YT*WVVARDVNTKQR-Z (SEQ ID NO: 4521), X-RLPWWR-C(Sx)-YT*WVVAREGNTKQR-Z (SEQ ID NO: 4522), X-RLPWWR-C(Sx)-YT*WVVAREVNTKQR-Z (SEQ ID NO: 4523), X-RLPWWR-C(Sx)-YT*WVVARQGNTKQR-Z (SEQ ID NO: 4524), X-RLPWWR-C(Sx)-YT*WVVARQVNTKQR-Z (SEQ ID NO: 4525), X-RLPWWR-C(Sx)-YT*WVVERDGNTKQR-Z (SEQ ID NO: 4526), X-RLPWWR-C(Sx)-YT*WVVERDVNTKQR-Z (SEQ ID NO: 4527), X-RLPWWR-C(Sx)-YT*WVVEREGNTKQR-Z (SEQ ID NO: 4528), X-RLPWWR-C(Sx)-YT*WVVEREVNTKQR-Z (SEQ ID NO: 4529), X-RLPWWR-C(Sx)-YT*WVVERQGNTKQR-Z (SEQ ID NO: 4530), and X-RLPWWR-C(Sx)-YT*WVVERQVNTKQR-Z (SEQ ID NO: 4531); wherein X— is H or acetyl; and Z is OH or NH2.

In some embodiments, the metal-binding compound of the present invention is selected from the group consisting of: X-RLR-C(Sx)-AS*FRRR-Z (SEQ ID NO: 4532), X-RNGKR-C(Sx)-PT*HTSRVGT-Z (SEQ ID NO: 4533), X-RNGKRT*P-C(Sx)-HTSRVGT-Z (SEQ ID NO: 4534), X-RNGKRTP-C(Sx)-HT*SRVGT-Z (SEQ ID NO: 4535), X-RNGKRTPT*H-C(Sx)-SRVGT-Z (SEQ ID NO: 4536), X-RNGKRTPTHT*S-C(Sx)-VGT-Z (SEQ ID NO: 4537), X-RNPWWR-C(Sx)-YT*WVVERDVNTKQR-Z (SEQ ID NO: 4538), X-RNR-C(Sx)-AS*FRRR-Z (SEQ ID NO: 4539), X-RPASVPPS*P-C(Sx)-LSRHS[pS]HQRR-Z (SEQ ID NO: 4540), X-RPASVPPS*P-C(Sx)-LSRHS[pS]HQR-Z (SEQ ID NO: 4541), X-RPASVPPSP-C(Sx)-LS*RHS[pS]HQRR-Z (SEQ ID NO: 4542), X-RPASVPPSP-C(Sx)-LS*RHS[pS]HQR-Z (SEQ ID NO: 4543), X-RPHFP-C(Sx)-FS*YSASGTA-Z (SEQ ID NO: 4544), X-RPPWWR-C(Sx)-YT*WVVERDVNTKQR-Z (SEQ ID NO: 4545), X-RPR-C(Sx)-AS*FRRR-Z (SEQ ID NO: 4546), X-RQPWWR-C(Sx)-YT*WVVERDVNTKQR-Z (SEQ ID NO: 4547), X-RQR-C(Sx)-AS*FRRR-Z (SEQ ID NO: 4548), X-RQREGFGRQS*M-C(Sx)-EKR-Z (SEQ ID NO: 4549), X-RR[Nle]-C(Sx)-AS*FRRR-Z (SEQ ID NO: 4550), X-RRA-C(Sx)-AS*FRRR-Z (SEQ ID NO: 4551), X-RR-C(Sx)-AS*FRRR-Z (SEQ ID NO: 4552), X-RR-C(Sx)-AS*FRR-Z (SEQ ID NO: 4553), X-RR-C(Sx)-AS*FR-Z (SEQ ID NO: 4554), X-RR-C(Sx)-LS*FAEG-Z (SEQ ID NO: 4555), X-RR-C(Sx)-LS*FAEPG-Z (SEQ ID NO: 4556), X-RRD-C(Sx)-AS*FRRR-Z (SEQ ID NO: 4557), X-RRE-C(Sx)-AS*FRRR-Z (SEQ ID NO: 4558), X-RRF-C(Sx)-AS*FRRR-Z (SEQ ID NO: 4559), X-RRG-C(Sx)-AS*FRRR-Z (SEQ ID NO: 4560), X-RRGGR-C(Sx)-RS*RSPDRRRR-Z (SEQ ID NO: 4561), X-RRGGRRRS*R-C(Sx)-PDRRRR-Z (SEQ ID NO: 4562), X-RRGGRRRSRS*P-C(Sx)-RRRR-Z (SEQ ID NO: 4563), X-RRH-C(Sx)-AS*FRRR-Z (SEQ ID NO: 4564), X-RRHYY-C(Sx)-DT*HTNTYYLRTFGHNTRR-Z (SEQ ID NO: 4565), X-RRHYYYDT*H-C(Sx)-NTYYLRTFGHNTRR-Z (SEQ ID NO: 4566), X-RRHYYYDTH-C(Sx)-NT*YYLRTFGHNTRR-Z (SEQ ID NO: 4567), X-RRHYYYDTHTNT*Y-C(Sx)-LRTFGHNTRR-Z (SEQ ID NO: 4568), X-RRHYYYDTHTNTYY-C(Sx)-RT*FGHNTRR-Z (SEQ ID NO: 4569), X-RRHYYYDTHTNTYYLRT*F-C(Sx)-HNTRR-Z (SEQ ID NO: 4570), X-RRI-C(Sx)-AS*FRRR-Z (SEQ ID NO: 4571), X-RRK-C(Sx)-AS*FRRR-Z (SEQ ID NO: 4572), X-RRK-C(Sx)-AT*RR[Nle]RF-Z (SEQ ID NO: 4573), X-RRK-C(Sx)-AT*RRMRF-Z (SEQ ID NO: 4574), X-RRK-C(Sx)-RT*[Nle]R[Nle]RF-Z (SEQ ID NO: 4575), X-RRK-C(Sx)-RT*MRMRF-Z (SEQ ID NO: 4576), X-RRKIAT*R-C(Sx)-[Nle]RF-Z (SEQ ID NO: 4577), X-RRKIAT*R-C(Sx)-MRF-Z (SEQ ID NO: 4578), X-RRKIRT*[Nle]-C(Sx)-[Nle]RF-Z (SEQ ID NO: 4579), X-RRKIRT*M-C(Sx)-MRF-Z (SEQ ID NO: 4580), X-RRL-C(Sx)-AS*FRRR-Z (SEQ ID NO: 4581), X-RRN-C(Sx)-AS*FRRR-Z (SEQ ID NO: 4582), X-RRPASVPPS*P-C(Sx)-LSRHS[pS]HQRR-Z (SEQ ID NO: 4583), X-RRPASVPPS*P-C(Sx)-LSRHS[pS]HQR-Z (SEQ ID NO: 4584), X-RRPASVPPSP-C(Sx)-LS*RHS[pS]HQRR-Z (SEQ ID NO: 4585), X-RRPASVPPSP-C(Sx)-LS*RHS[pS]HQR-Z (SEQ ID NO: 4586), X-RRP-C(Sx)-AS*FRRR-Z (SEQ ID NO: 4587), and X-RRPWWR-C(Sx)-YT*WVVERDVNTKQR-Z (SEQ ID NO: 4588); wherein X— is H or acetyl; and Z is OH or NH2.

In some embodiments, the metal-binding compound of the present invention is selected from the group consisting of: X-RRQ-C(Sx)-AS*FRRR-Z (SEQ ID NO: 4589), X-RRR-C(Sx)-[Nle]S*FRRR-Z (SEQ ID NO: 4590), X-RRR-C(Sx)-A[Nle]FRRR-Z (SEQ ID NO: 4591), X-RRR-C(Sx)-AS*[Nle]RRR-Z (SEQ ID NO: 4592), X-RRR-C(Sx)-AS*ARRR-Z (SEQ ID NO: 4593), X-RRR-C(Sx)-AS*DRRR-Z (SEQ ID NO: 4594), X-RRR-C(Sx)-AS*ERRR-Z (SEQ ID NO: 4595), X-RRR-C(Sx)-AS*F[Nle]RR-Z (SEQ ID NO: 4596), X-RRR-C(Sx)-AS*FARR-Z (SEQ ID NO: 4597), X-RRR-C(Sx)-AS*FDRR-Z (SEQ ID NO: 4598), X-RRR-C(Sx)-AS*FERR-Z (SEQ ID NO: 4599), X-RRR-C(Sx)-AS*FFRR-Z (SEQ ID NO: 4600), X-RRR-C(Sx)-AS*FGRR-Z (SEQ ID NO: 4601), X-RRR-C(Sx)-AS*FHRR-Z (SEQ ID NO: 4602), X-RRR-C(Sx)-AS*FIRR-Z (SEQ ID NO: 4603), X-RRR-C(Sx)-AS*FKRR-Z (SEQ ID NO: 4604), X-RRR-C(Sx)-AS*FLRR-Z (SEQ ID NO: 4605), X-RRR-C(Sx)-AS*FNRR-Z (SEQ ID NO: 4606), X-RRR-C(Sx)-AS*FPRR-Z (SEQ ID NO: 4607), X-RRR-C(Sx)-AS*FQRR-Z (SEQ ID NO: 4608), X-RRR-C(Sx)-AS*FR[Nle]R-Z (SEQ ID NO: 4609), X-RRR-C(Sx)-AS*FRAR-Z (SEQ ID NO: 4610), X-RRR-C(Sx)-AS*FRDR-Z (SEQ ID NO: 4611), X-RRR-C(Sx)-AS*FRER-Z (SEQ ID NO: 4612), X-RRR-C(Sx)-AS*FRFR-Z (SEQ ID NO: 4613), X-RRR-C(Sx)-AS*FRGR-Z (SEQ ID NO: 4614), X-RRR-C(Sx)-AS*FRHR-Z (SEQ ID NO: 4615), X-RRR-C(Sx)-AS*FRIR-Z (SEQ ID NO: 4616), X-RRR-C(Sx)-AS*FRKR-Z (SEQ ID NO: 4617), X-RRR-C(Sx)-AS*FRLR-Z (SEQ ID NO: 4618), X-RRR-C(Sx)-AS*FRNR-Z (SEQ ID NO: 4619), X-RRR-C(Sx)-AS*FRPR-Z (SEQ ID NO: 4620), X-RRR-C(Sx)-AS*FRQR-Z (SEQ ID NO: 4621), X-RRR-C(Sx)-AS*FRR[Nle]-Z (SEQ ID NO: 4622), X-RRR-C(Sx)-AS*FRRA-Z (SEQ ID NO: 4623), X-RRR-C(Sx)-AS*FRRD-Z (SEQ ID NO: 4624), X-RRR-C(Sx)-AS*FRRE-Z (SEQ ID NO: 4625), X-RRR-C(Sx)-AS*FRRF-Z (SEQ ID NO: 4626), X-RRR-C(Sx)-AS*FRRG-Z (SEQ ID NO: 4627), X-RRR-C(Sx)-AS*FRRH-Z (SEQ ID NO: 4628), X-RRR-C(Sx)-AS*FRRI-Z (SEQ ID NO: 4629), X-RRR-C(Sx)-AS*FRRK-Z (SEQ ID NO: 4630), X-RRR-C(Sx)-AS*FRRL-Z (SEQ ID NO: 4631), X-RRR-C(Sx)-AS*FRRN-Z (SEQ ID NO: 4632), X-RRR-C(Sx)-AS*FRRP-Z (SEQ ID NO: 4633), X-RRR-C(Sx)-AS*FRRQ-Z (SEQ ID NO: 4634), X-RRR-C(Sx)-AS*FRRR-Z (SEQ ID NO: 4635), X-RRR-C(Sx)-AS*FRRV-Z (SEQ ID NO: 4636), X-RRR-C(Sx)-AS*FRRW-Z (SEQ ID NO: 4637), X-RRR-C(Sx)-AS*FRR-Z (SEQ ID NO: 4638), X-RRR-C(Sx)-AS*FRVR-Z (SEQ ID NO: 4639), X-RRR-C(Sx)-AS*FRWR-Z (SEQ ID NO: 4640), X-RRR-C(Sx)-AS*FR-Z (SEQ ID NO: 4641), X-RRR-C(Sx)-AS*FVRR-Z (SEQ ID NO: 4642), X-RRR-C(Sx)-AS*FWRR-Z (SEQ ID NO: 4643), X-RRR-C(Sx)-AS*GRRR-Z (SEQ ID NO: 4644), X-RRR-C(Sx)-AS*HRRR-Z (SEQ ID NO: 4645), X-RRR-C(Sx)-AS*IRRR-Z (SEQ ID NO: 4646), X-RRR-C(Sx)-AS*KRRR-Z (SEQ ID NO: 4647), X-RRR-C(Sx)-AS*LRRR-Z (SEQ ID NO: 4648), X-RRR-C(Sx)-AS*NRRR-Z (SEQ ID NO: 4649), X-RRR-C(Sx)-AS*PRRR-Z (SEQ ID NO: 4650), X-RRR-C(Sx)-AS*QRRR-Z (SEQ ID NO: 4651), X-RRR-C(Sx)-AS*RRRR-Z (SEQ ID NO: 4652), X-RRR-C(Sx)-AS*VRRR-Z (SEQ ID NO: 4653), X-RRR-C(Sx)-AS*WRRR-Z (SEQ ID NO: 4654), X-RRR-C(Sx)-DS*FRRR-Z (SEQ ID NO: 4655), X-RRR-C(Sx)-ES*FRRR-Z (SEQ ID NO: 4656), X-RRR-C(Sx)-FS*FRRR-Z (SEQ ID NO: 4657), X-RRR-C(Sx)-FS*LRRKA-Z (SEQ ID NO: 4658), X-RRR-C(Sx)-GS*FRRR-Z (SEQ ID NO: 4659), X-RRR-C(Sx)-HS*FRRR-Z (SEQ ID NO: 4660), X-RRR-C(Sx)-IS*FRRR-Z (SEQ ID NO: 4661), X-RRR-C(Sx)-KS*FRRR-Z (SEQ ID NO: 4662), X-RRR-C(Sx)-LS*FRRR-Z (SEQ ID NO: 4663), X-RRR-C(Sx)-NS*FRRR-Z (SEQ ID NO: 4664), X-RRR-C(Sx)-PS*FRRR-Z (SEQ ID NO: 4665), X-RRR-C(Sx)-QS*FRRR-Z (SEQ ID NO: 4666), X-RRR-C(Sx)-RS*FRRR-Z (SEQ ID NO: 4667), X-RRR-C(Sx)-VS*FRRR-Z (SEQ ID NO: 4668), X-RRR-C(Sx)-WS*FRRR-Z (SEQ ID NO: 4669), X-RRRLS-C(Sx)-VS*LTGVSTI-Z (SEQ ID NO: 1157), X-RRRLSNVS*L-C(Sx)-GVSTI-Z (SEQ ID NO: 1156), X-RRRPASVPPS*P-C(Sx)-LSRHS[pS]HQRR-Z (SEQ ID NO: 4670), X-RRRPASVPPS*P-C(Sx)-LSRHS[pS]HQR-Z (SEQ ID NO: 4671), X-RRRPASVPPSP-C(Sx)-LS*RHS[pS]HQRR-Z (SEQ ID NO: 4672), X-RRRPASVPPSP-C(Sx)-LS*RHS[pS]HQR-Z (SEQ ID NO: 4673), X-RRRQFS*L-C(Sx)-RKA-Z (SEQ ID NO: 4674), X-RRV-C(Sx)-AS*FRRR-Z (SEQ ID NO: 4675), X-RRVSGNNS*P-C(Sx)-LSNGG-Z (SEQ ID NO: 1112), X-RRVSGNNSP-C(Sx)-LS*NGG-Z (SEQ ID NO: 4676), X-RRW-C(Sx)-AS*FRRR-Z (SEQ ID NO: 4677), X-RSKSAPTS*P-C(Sx)-DQEIK-Z (SEQ ID NO: 1052), X-RSRSRS*R-C(Sx)-R-Z (SEQ ID NO: 4678), X-RSRSRSRS*R-C(Sx)-R-Z (SEQ ID NO: 4679), X-RSRSRSRSNS*R-C(Sx)-RSYSPRRS-Z (SEQ ID NO: 4680), X-RSRSRSRSNSR-C(Sx)-RS*YSPRRS-Z (SEQ ID NO: 4681), X-RSRSRSRSNSRS*R-C(Sx)-YSPRRS-Z (SEQ ID NO: 4682), X-RSRSRSRSNSRSR-C(Sx)-YS*PRRS-Z (SEQ ID NO: 4683), X-RSRSRSRSRS*R-C(Sx)-RAR-Z (SEQ ID NO: 4684), X-RSRSRSRSRS*R-C(Sx)-RGR-Z (SEQ ID NO: 4685), X-RSRSRSRSRS*R-C(Sx)-R-Z (SEQ ID NO: 4686), X-RSRSRSRSRSNSRSRS*Y-C(Sx)-PR-Z (SEQ ID NO: 4687), X-RSRSRSRSRSRS*R-C(Sx)-R-Z (SEQ ID NO: 4688), X-RTDSLAAT*P-C(Sx)-AAK-Z (SEQ ID NO: 1111), X-RVG-C(Sx)-AS*SLENTVDLHISNSHP-Z (SEQ ID NO: 4689), X-RVGGASS*L-C(Sx)-NTVDLHISNSHP-Z (SEQ ID NO: 4690), X-RVGGASSL-C(Sx)-NT*VDLHISNSHP-Z (SEQ ID NO: 4691), X-RVPWWR-C(Sx)-YT*WVVERDVNTKQR-Z (SEQ ID NO: 4692), X-RVR-C(Sx)-AS*FRRR-Z (SEQ ID NO: 4693), X-RWPWWR-C(Sx)-YT*WVVERDVNTKQR-Z (SEQ ID NO: 4694), and X-RWR-C(Sx)-AS*FRRR-Z (SEQ ID NO: 4695); wherein X— is H or acetyl; and Z is OH or NH2.

In some embodiments, the metal-binding compound of the present invention is selected from the group consisting of: X-S*R-C(Sx)-LSVSSLPGLED-Z (SEQ ID NO: 4696), X-S*R-C(Sx)-SSPHQ[pS]EDEEE-Z (SEQ ID NO: 4697), X-SAKS*R-C(Sx)-QTAPVP[Nle]PD-Z (SEQ ID NO: 4698), X-SAKSR-C(Sx)-QT*APVP[Nle]PD-Z (SEQ ID NO: 4699), X-SAKSRLQT*A-C(Sx)-VP[Nle]PD-Z (SEQ ID NO: 4700), X-SANLLS*P-C(Sx)-PA-Z (SEQ ID NO: 4701), X-SEGLS*M-C(Sx)-NYIGLINRIKK-Z (SEQ ID NO: 4702), X-SFITPPTT*P-C(Sx)-LRRHT-Z (SEQ ID NO: 1092), X-SGDED-C(Sx)-SS*IADMDFSAL-Z (SEQ ID NO: 4703), X-SGDEDFSS*I-C(Sx)-DMDFSALLSQ-Z (SEQ ID NO: 4704), X-SGDY[Nle]P[Nle]S*P-C(Sx)-SVSAP-Z (SEQ ID NO: 4705), X-SGLS*R-C(Sx)-DSPEPFGHY-Z (SEQ ID NO: 4706), X-SGLSR-C(Sx)-DS*PEPFGHY-Z (SEQ ID NO: 4707), X-SGLSRRDS*P-C(Sx)-PFGHY-Z (SEQ ID NO: 4708), X-SGQL-C(Sx)-DS*[Nle]ANSFV-Z (SEQ ID NO: 4709), X-SGQLIDS*[Nle]-C(Sx)-NSFV-Z (SEQ ID NO: 4710), X-SGQLIDS[Nle]ANS*F-C(Sx)-G-Z (SEQ ID NO: 4711), X-SGQLIDS[Nle]-C(Sx)-NS*FV-Z (SEQ ID NO: 4712), X-SGRPR-C(Sx)-TS*FAESSKP-Z (SEQ ID NO: 4713), X-SGRPRTTS*F-C(Sx)-ESSKP-Z (SEQ ID NO: 4714), X-SGVEV-C(Sx)-VT*PTRTEII-Z (SEQ ID NO: 1061), X-SGVEVRVT*P-C(Sx)-RTEII-Z (SEQ ID NO: 1060), X-SGVEVRVTP-C(Sx)-RT*EII-Z (SEQ ID NO: 4715), X-SIKS*E-C(Sx)-ISPPRDR[Nle]T-Z (SEQ ID NO: 4716), X-SIKSE-C(Sx)-IS*PPRDR[Nle]T-Z (SEQ ID NO: 4717), X-SIKSEPIS*P-C(Sx)-RDR[Nle]T-Z (SEQ ID NO: 4718), X-SIMQQ-C(Sx)-DS*PHVVKYYKKK-Z (SEQ ID NO: 4719), X-SKS*S-C(Sx)-NGTSGDREDK-Z (SEQ ID NO: 4720), X-SLD-C(Sx)-SS*LSLSLSSANSLYDD-Z (SEQ ID NO: 4721), X-SLDSSSLS*L-C(Sx)-LSSANSLYDD-Z (SEQ ID NO: 4722), X-SLDSSSLSL-C(Sx)-LS*SANSLYDD-Z (SEQ ID NO: 4723), X-SNGRR-C(Sx)-RS*PTQSFRF-Z (SEQ ID NO: 1037), X-SNGRRKRS*P-C(Sx)-QSFRF-Z (SEQ ID NO: 1036), X-SNGRRKRSP-C(Sx)-QS*FRF-Z (SEQ ID NO: 4724), X-SNSLKKSS*A-C(Sx)-LKKIL-Z (SEQ ID NO: 1180), X-SNT*GQ-C(Sx)-ISPG[Nle]KRRI-Z (SEQ ID NO: 4725), X-SNTGQAIS*P-C(Sx)-[Nle]KRRI-Z (SEQ ID NO: 4726), X-SNTGQAIS*PG-C(Sx)-KRRI-Z (SEQ ID NO: 4727), X-SNTGQ-C(Sx)-IS*PG[Nle]KRRI-Z (SEQ ID NO: 4728), X-SPAR-C(Sx)-SS*YSEANEP-Z (SEQ ID NO: 4729), X-SPLSDPSS*P-C(Sx)-ASEDF-Z (SEQ ID NO: 1182), X-SPLSDPSSP-C(Sx)-AS*EDF-Z (SEQ ID NO: 4730), X-SPS-C(Sx)-RS*RSRSRSRSPGRPSK-Z (SEQ ID NO: 4731), X-SPSRRS*R-C(Sx)-RSRSRSPGRPSK-Z (SEQ ID NO: 4732), X-SPSRRSR-C(Sx)-RS*RSRSPGRPSK-Z (SEQ ID NO: 4733), X-SPSRRSRS*R-C(Sx)-RSRSPGRPSK-Z (SEQ ID NO: 4734), X-SPSRRSRSR-C(Sx)-RS*RSPGRPSK-Z (SEQ ID NO: 4735), X-SPSRRSRSRS*R-C(Sx)-RSPGRPSK-Z (SEQ ID NO: 4736), X-SPSRRSRSRSR-C(Sx)-RS*PGRPSK-Z (SEQ ID NO: 4737), X-SPSRRSRSRSRS*R-C(Sx)-PGRPSK-Z (SEQ ID NO: 4738), X-SPSRRSRSRSRSRS*P-C(Sx)-RPSK-Z (SEQ ID NO: 4739), X-SR-C(Sx)-LS*VSSLPGLED-Z (SEQ ID NO: 4740), X-SR-C(Sx)-SS*PHQ[pS]EDEEE-Z (SEQ ID NO: 4741), X-SRDPVART*S-C(Sx)-LQTPA-Z (SEQ ID NO: 1104), X-SRLRD-C(Sx)-PS*APLEAPE-Z (SEQ ID NO: 1171), X-SRRS*G-C(Sx)-SSPSYVAVT-Z (SEQ ID NO: 4742), X-SRRSG-C(Sx)-SS*PSYVAVT-Z (SEQ ID NO: 1063), X-SRRSGLSS*P-C(Sx)-YVAVT-Z (SEQ ID NO: 1062), X-SRSHSAKT*P-C(Sx)-FSVQS-Z (SEQ ID NO: 1069), X-SRSHSAKT*PG-C(Sx)-SVQS-Z (SEQ ID NO: 4743), X-SRSHSAKTP-C(Sx)-FS*VQS-Z (SEQ ID NO: 4744), X-SRSHS-C(Sx)-KT*PGFSVQS-Z (SEQ ID NO: 4745), X-SRSRS*R-C(Sx)-RAPGRPSKG-Z (SEQ ID NO: 4746), X-SRSRS*R-C(Sx)-RGPGRPSKG-Z (SEQ ID NO: 4747), X-SRSRS*R-C(Sx)-RSPGRPSKG-Z (SEQ ID NO: 4748), X-SRSRSNSRSRS*Y-C(Sx)-PRRSRGS-Z (SEQ ID NO: 4749), X-SRSRSNSRSRSYS*P-C(Sx)-RSRGS-Z (SEQ ID NO: 4750), X-SRSRSNSRSRSYSP-C(Sx)-RS*RGS-Z (SEQ ID NO: 4751), X-SRSRSR-C(Sx)-RS*PGRPSKG-Z (SEQ ID NO: 4752), X-SRSRSRSNSRSRSYS*P-C(Sx)-RSR-Z (SEQ ID NO: 4753), X-SRSRSRSNSRSRSYSP-C(Sx)-RS*R-Z (SEQ ID NO: 4754), X-SRSRSRSRS*P-C(Sx)-RPSKGR-Z (SEQ ID NO: 4755), X-SRSRSRSRS*P-C(Sx)-RPSKG-Z (SEQ ID NO: 4756), X-SRSRSRSRSNSRSRS*Y-C(Sx)-PRR-Z (SEQ ID NO: 4757), X-SRSRSRSRSNSRSRSYS*P-C(Sx)-R-Z (SEQ ID NO: 4758), X-SSKIRRLS*A-C(Sx)-KQQ-Z (SEQ ID NO: 4759), X-SSKRAKAKTTKKRPQRAT*S-C(Sx)-VFS-Z (SEQ ID NO: 4760), X-SSKRAKAKTTKKRPQRATS*N-C(Sx)-FS-Z (SEQ ID NO: 998), X-SSKRAKAKTTKKRPQRATSN-C(Sx)-FS*-Z (SEQ ID NO: 4761), X-SSLET-C(Sx)-ST*QELYSIP-Z (SEQ ID NO: 4762), X-SSPS-C(Sx)-RS*RSRSRSRS-Z (SEQ ID NO: 4763), X-SSPSRR-C(Sx)-RS*RSRSRS-Z (SEQ ID NO: 4764), X-SSPSRRS*R-C(Sx)-RSRSRS-Z (SEQ ID NO: 4765), X-SSPSRRSR-C(Sx)-RS*RSRS-Z (SEQ ID NO: 4766), X-SSPSRRSRSRS*R-C(Sx)-RA-Z (SEQ ID NO: 4767), X-SSPSRRSRSRS*R-C(Sx)-RG-Z (SEQ ID NO: 4768), X-SSPSRRSRSRS*R-C(Sx)-RS-Z (SEQ ID NO: 4769), X-SSPSRRSRSRSR-C(Sx)-RS*-Z (SEQ ID NO: 4770), and X-SSPSRRSRSRSRS*R-C(Sx)-Z (SEQ ID NO: 4771); wherein X— is H or acetyl; and Z is OH or NH2.

In some embodiments, the metal-binding compound of the present invention is selected from the group consisting of: X-SSPSRRSRSRSRSRS*P-C(Sx)-RPSKGR-Z (SEQ ID NO: 4772), X-SSPSRRSRSRSRSRS*P-C(Sx)-RPSKG-Z (SEQ ID NO: 4773), X-STPSEPLS*P-C(Sx)-SSLGE-Z (SEQ ID NO: 4774), X-STPSEPLSP-C(Sx)-SS*LGE-Z (SEQ ID NO: 4775), X-STSVT-C(Sx)-HS*PSSPVGSKKK-Z (SEQ ID NO: 4776), X-SYYGRDRS*P-C(Sx)-RRATA-Z (SEQ ID NO: 4777), X-TDGEDADYT-C(Sx)-FT*NQQ-Z (SEQ ID NO: 4778), X-TEERL-C(Sx)-SS*PVYEDAA-Z (SEQ ID NO: 1035), X-TFDS*L-C(Sx)-SSPSSATPH-Z (SEQ ID NO: 4779), X-TFDSL-C(Sx)-SS*PSSATPH-Z (SEQ ID NO: 1065), X-TFDSLPSS*P-C(Sx)-SATPH-Z (SEQ ID NO: 1064), X-TFGNKLDT*F-C(Sx)-GSP-Z (SEQ ID NO: 4780), X-TFGNKLDTF-C(Sx)-GS*P-Z (SEQ ID NO: 4781), X-TFLPVPEYI-C(Sx)-QS*VPK-Z (SEQ ID NO: 4782), X-TKLKPPRT*P-C(Sx)-PPSRK-Z (SEQ ID NO: 1093), X-TPPEE-C(Sx)-PS*PSASSLG-Z (SEQ ID NO: 1027), X-TPPEELPS*P-C(Sx)-ASSLG-Z (SEQ ID NO: 1026), X-TPPEELPSP-C(Sx)-AS*SLG-Z (SEQ ID NO: 4783), X-TPPQEHIS*P-C(Sx)-ITNEV-Z (SEQ ID NO: 1053), X-TPPQEHISP-C(Sx)-IT*NEV-Z (SEQ ID NO: 4784), X-TPVVS-C(Sx)-AT*PTLPGQG-Z (SEQ ID NO: 4785), X-TTGT*K-C(Sx)-NTPTSSVPSKKK-Z (SEQ ID NO: 4786), X-TTGTK-C(Sx)-NT*PTSSVPSKKK-Z (SEQ ID NO: 4787), X-TTLHR-C(Sx)-VS*PGAPQRP-Z (SEQ ID NO: 1140), X-TTLHRNVS*P-C(Sx)-APQRP-Z (SEQ ID NO: 1139), X-TTLHRNVS*PG-C(Sx)-PQRP-Z (SEQ ID NO: 4788), X-TVGNKLDT*F-C(Sx)-GSP-Z (SEQ ID NO: 4789), X-TVGNKLDTF-C(Sx)-GS*P-Z (SEQ ID NO: 4790), X-VA-C(Sx)-QT*P[pT]PTRFLKN-Z (SEQ ID NO: 1023), X-VADQTPT*P-C(Sx)-RFLKN-Z (SEQ ID NO: 1022), X-VAKTTKKRPQRATS*N-C(Sx)-FS-Z (SEQ ID NO: 4791), X-VAKTTKKRPQRATSN-C(Sx)-FS*-Z (SEQ ID NO: 4792), X-VANPLMA-C(Sx)-GT*LTRRHQNGRF-Z (SEQ ID NO: 4793), X-V-C(Sx)-DS*LRRKAK-Z (SEQ ID NO: 4794), X-V-C(Sx)-LS*PGPF-Z (SEQ ID NO: 4795), X-VDGFT-C(Sx)-KS*VRKAQRQ-Z (SEQ ID NO: 4796), X-VDGFTRKS*V-C(Sx)-KAQRQ-Z (SEQ ID NO: 4797), X-VDLHISNS*H-C(Sx)-LSLTSDQYK-Z (SEQ ID NO: 4798), X-VDLHISNSH-C(Sx)-LS*LTSDQYK-Z (SEQ ID NO: 4799), X-VDLHISNSHPL-C(Sx)-LT*SDQYK-Z (SEQ ID NO: 4800), X-VEVDP[Nle]LT*P-C(Sx)-ERHLN-Z (SEQ ID NO: 4801), X-VEVDP-C(Sx)-LT*PEERHLN-Z (SEQ ID NO: 1045), X-VKAEPAHT*A-C(Sx)-SVAAK-Z (SEQ ID NO: 1173), X-VKRPQRAT*S-C(Sx)-VFAMF-Z (SEQ ID NO: 4802), X-VKS*P-C(Sx)-KEKAKSPEK-Z (SEQ ID NO: 4803), X-VKSPAKEKAKS*P-C(Sx)-K-Z (SEQ ID NO: 4804), X-VKSPAKEK-C(Sx)-KS*PEK-Z (SEQ ID NO: 4805), X-VLLR-C(Sx)-SS*RRIRR-Z (SEQ ID NO: 4806), X-VLPWWR-C(Sx)-YT*WVVERDVNTKQR-Z (SEQ ID NO: 4807), X-VLSRQ-C(Sx)-TS*PVSGING-Z (SEQ ID NO: 1051), X-VLSRQITS*P-C(Sx)-SGING-Z (SEQ ID NO: 1050), X-VNTRA-C(Sx)-PS*QHSSPAV-Z (SEQ ID NO: 1177), X-VP-C(Sx)-LT*PGGRR-Z (SEQ ID NO: 4808), X-VQGI-C(Sx)-FS*QPTSPDHAKKK-Z (SEQ ID NO: 4809), X-VQREGFGRQS*M-C(Sx)-EKR-Z (SEQ ID NO: 4810), X-VRR-C(Sx)-AS*FRRR-Z (SEQ ID NO: 4811), X-VSGQL-C(Sx)-DS*MANSFVGTRSYKKK-Z (SEQ ID NO: 1003), X-VSYEDPPT*A-C(Sx)-AAVEW-Z (SEQ ID NO: 1075), and X-WAKTTKKRPQRATS*N-C(Sx)-FS-Z (SEQ ID NO: 4812); wherein X is H or acetyl; and Z is OH or NH2.

In some embodiments, the metal-binding compound of the present invention is selected from the group consisting of: X-WAKTTKKRPQRATSN-C(Sx)-FS*-Z (SEQ ID NO: 4813), X-WANPLMA-C(Sx)-GT*LTRRHQNGRF-Z (SEQ ID NO: 4814), X-W-C(Sx)-DS*LRRKAK-Z (SEQ ID NO: 4815), X-W-C(Sx)-LS*PGPF-Z (SEQ ID NO: 4816), X-WD-C(Sx)-DS*DDDDDAAAKKK-Z (SEQ ID NO: 1195), X-WD-C(Sx)-DS*DDDDDAAA-Z (SEQ ID NO: 1194), X-WKRPQRAT*S-C(Sx)-VFAMF-Z (SEQ ID NO: 4817), X-WLLR-C(Sx)-SS*RRIRR-Z (SEQ ID NO: 4818), X-WLPWWR-C(Sx)-YT*WVVERDVNTKQR-Z (SEQ ID NO: 4819), X-WQREGFGRQS*M-C(Sx)-EKR-Z (SEQ ID NO: 4820), X-WR-C(Sx)-YT*LRRARQGNTKQR-Z (SEQ ID NO: 4821), X-WRR-C(Sx)-AS*FRRR-Z (SEQ ID NO: 4822), X-Y[Nle]P[Nle]SPKS*V-C(Sx)-APQQI-Z (SEQ ID NO: 4823), X-YANWMAAS*I-C(Sx)-LDGKKK-Z (SEQ ID NO: 4824), X-YANWM-C(Sx)-AS*IYLDGKKK-Z (SEQ ID NO: 4825), X-YKRRYVET*P-C(Sx)-VHISS-Z (SEQ ID NO: 1097), X-YRRAAVPPSPSLS*R-C(Sx)-SSPHQ[pS]EDEEE-Z (SEQ ID NO: 4826), X-YRRAAVPPSPSLSR-C(Sx)-SS*PHQ[pS]EDEEE-Z (SEQ ID NO: 4827), X-YSHKGHLS*E-C(Sx)-LVTKW-Z (SEQ ID NO: 1168), X-[Nle]EEEEY*I-C(Sx)-Z (SEQ ID NO: 4828), X-[Nle]EEEY*I-C(Sx)-IV-Z (SEQ ID NO: 4829), X-[Nle]EPIY*I-C(Sx)-VP-Z (SEQ ID NO: 4830), X-[Nle]VSETDDY*A-C(Sx)-IIDEE-Z (SEQ ID NO: 4831), X-ADEY*L-C(Sx)-PQQ-Z (SEQ ID NO: 4832), X-ADPDHDHTGFLTEY*V-C(Sx)-TRWRR-Z (SEQ ID NO: 4833), X-AEE-C(Sx)-IY*GEFEAKKKK-Z (SEQ ID NO: 4834), X-AEEEEY*I-C(Sx)-Z (SEQ ID NO: 4835), X-AEEEY*I-C(Sx)-IV-Z (SEQ ID NO: 4836), X-AENAEY*L-C(Sx)-VA-Z (SEQ ID NO: 945), X-AGKEQTYY*Q-C(Sx)-RHLGD-Z (SEQ ID NO: 981), X-AKAA-C(Sx)-GY*VKPQIKQVV-Z (SEQ ID NO: 4837), X-AKAADGY*V-C(Sx)-PQIKQVV-Z (SEQ ID NO: 4838), X-AKA-C(Sx)-DGY*VKPQIKQVV-Z (SEQ ID NO: 4839), X-ARKRERAY*SA[dP]-C(Sx)-G-Z (SEQ ID NO: 4840), X-ARKRERAY*SD[dP]-C(Sx)-G-Z (SEQ ID NO: 4841), X-ARKRERAY*SE-C(Sx)-G-Z (SEQ ID NO: 4842), X-ARKRERAY*SF[Nle]-C(Sx)-G-Z (SEQ ID NO: 4843), X-ARPLVEFY*E-C(Sx)-IKKYE-Z (SEQ ID NO: 927), X-AVLADVSY*L-C(Sx)-AMEKS-Z (SEQ ID NO: 926), X-AVSETDDY*A-C(Sx)-IIDEE-Z (SEQ ID NO: 4844), X-C(Sx)-FY*TLRRARQGNTKQR-Z (SEQ ID NO: 4845), X-DADEY*L-C(Sx)-PQQG-Z (SEQ ID NO: 939), X-DE-C(Sx)-DY*EEPDEP-Z (SEQ ID NO: 4846), X-DEEDY*E-C(Sx)-PDEP-Z (SEQ ID NO: 4847), X-DEEEEY*I-C(Sx)-Z (SEQ ID NO: 4848), X-DEEEY*I-C(Sx)-IV-Z (SEQ ID NO: 4849), X-DEPE-C(Sx)-DY*EEVLEPED-Z (SEQ ID NO: 4850), X-DEPE-C(Sx)-DY*FEWLEPED-Z (SEQ ID NO: 4851), X-DEPEGDY*E-C(Sx)-VLEPED-Z (SEQ ID NO: 4852), X-DEPEGDY*F-C(Sx)-WLEPED-Z (SEQ ID NO: 4853), X-DGDGQVNY*E-C(Sx)-FVQMM-Z (SEQ ID NO: 929), X-DGSDHPYY*N-C(Sx)-IPSKM-Z (SEQ ID NO: 978), X-DLI[Nle]K-C(Sx)-DY*ELVSTKPTR-Z (SEQ ID NO: 4854), X-DLI[Nle]KEDY*E-C(Sx)-VSTKPTR-Z (SEQ ID NO: 4855), X-DLIMK-C(Sx)-DY*ELVSTKPTR-Z (SEQ ID NO: 4856), X-DLIMKEDY*E-C(Sx)-VSTKPTR-Z (SEQ ID NO: 4857), X-DNDP-C(Sx)-EY*ITLDED-Z (SEQ ID NO: 4858), X-DNDP-C(Sx)-RY*IRTEDE-Z (SEQ ID NO: 4859), X-DNDPDEY*I-C(Sx)-LDED-Z (SEQ ID NO: 4860), X-DNDPDRY*I-C(Sx)-TEDE-Z (SEQ ID NO: 4861), X-DRHEQEDEPE-C(Sx)-DY*EEVLEPE-Z (SEQ ID NO: 4862), X-DRHEQEDEPE-C(Sx)-DY*FEWLEPE-Z (SEQ ID NO: 4863), X-DRHEQEDEPEGDY*E-C(Sx)-VLEPE-Z (SEQ ID NO: 4864), X-DRHEQEDEPEGDY*F-C(Sx)-WLEPE-Z (SEQ ID NO: 4865), and X-DVSETDDY*A-C(Sx)-IIDEE-Z (SEQ ID NO: 4866); wherein X is H or acetyl; and Z is OH or NH2.

In some embodiments, the metal-binding compound of the present invention is selected from the group consisting of: X-DVVDADEY*L-C(Sx)-PQQGF-Z (SEQ ID NO: 938), X-ED-C(Sx)-DY*EWPSA-Z (SEQ ID NO: 4867), X-EDE-C(Sx)-IY*EELDEP-Z (SEQ ID NO: 4868), X-EDEDIY*E-C(Sx)-LDEP-Z (SEQ ID NO: 4869), X-EDEG-C(Sx)-RY*LKLEED-Z (SEQ ID NO: 4870), X-EDEGDRY*L-C(Sx)-LEED-Z (SEQ ID NO: 4871), X-EDGGDDIY*E-C(Sx)-IIKVE-Z (SEQ ID NO: 986), X-EDPDY*E-C(Sx)-PSA-Z (SEQ ID NO: 4872), X-EDPDY*F-C(Sx)-FG-Z (SEQ ID NO: 251), X-EDPDY*F-C(Sx)-FK-Z (SEQ ID NO: 252), X-EDPDY*F-C(Sx)-FP-Z (SEQ ID NO: 253), X-EDPDY*F-C(Sx)-IG-Z (SEQ ID NO: 254), X-EDPDY*F-C(Sx)-IK-Z (SEQ ID NO: 255), X-EDPDY*F-C(Sx)-IP-Z (SEQ ID NO: 256), X-EDPDY*F-C(Sx)-MG-Z (SEQ ID NO: 257), X-EDPDY*F-C(Sx)-MK-Z (SEQ ID NO: 258), X-EDPDY*F-C(Sx)-MP-Z (SEQ ID NO: 259), X-EDPDY*F-C(Sx)-VG-Z (SEQ ID NO: 260), X-EDPDY*F-C(Sx)-VK-Z (SEQ ID NO: 261), X-EDPDY*F-C(Sx)-VP-Z (SEQ ID NO: 262), X-EDPDY*I-C(Sx)-FG-Z (SEQ ID NO: 263), X-EDPDY*I-C(Sx)-FK-Z (SEQ ID NO: 264), X-EDPDY*I-C(Sx)-FP-Z (SEQ ID NO: 265), X-EDPDY*I-C(Sx)-IG-Z (SEQ ID NO: 266), X-EDPDY*I-C(Sx)-IK-Z (SEQ ID NO: 267), X-EDPDY*I-C(Sx)-IP-Z (SEQ ID NO: 268), X-EDPDY*I-C(Sx)-MG-Z (SEQ ID NO: 269), X-EDPDY*I-C(Sx)-MK-Z (SEQ ID NO: 270), X-EDPDY*I-C(Sx)-MP-Z (SEQ ID NO: 271), X-EDPDY*I-C(Sx)-VG-Z (SEQ ID NO: 272), X-EDPDY*I-C(Sx)-VK-Z (SEQ ID NO: 273), X-EDPDY*I-C(Sx)-VP-Z (SEQ ID NO: 274), X-EDPDY*V-C(Sx)-FG-Z (SEQ ID NO: 275), X-EDPDY*V-C(Sx)-FK-Z (SEQ ID NO: 276), X-EDPDY*V-C(Sx)-FP-Z (SEQ ID NO: 277), X-EDPDY*V-C(Sx)-IG-Z (SEQ ID NO: 278), X-EDPDY*V-C(Sx)-IK-Z (SEQ ID NO: 279), X-EDPDY*V-C(Sx)-IP-Z (SEQ ID NO: 280), X-EDPDY*V-C(Sx)-MG-Z (SEQ ID NO: 281), X-EDPDY*V-C(Sx)-MK-Z (SEQ ID NO: 282), X-EDPDY*V-C(Sx)-MP-Z (SEQ ID NO: 283), X-EDPDY*V-C(Sx)-VG-Z (SEQ ID NO: 284), X-EDPDY*V-C(Sx)-VK-Z (SEQ ID NO: 285), X-EDPDY*V-C(Sx)-VP-Z (SEQ ID NO: 286), X-EDPEY*F-C(Sx)-FG-Z (SEQ ID NO: 287), X-EDPEY*F-C(Sx)-FK-Z (SEQ ID NO: 288), X-EDPEY*F-C(Sx)-FP-Z (SEQ ID NO: 289), X-EDPEY*F-C(Sx)-IG-Z (SEQ ID NO: 290), X-EDPEY*F-C(Sx)-IK-Z (SEQ ID NO: 291), X-EDPEY*F-C(Sx)-IP-Z (SEQ ID NO: 292), X-EDPEY*F-C(Sx)-MG-Z (SEQ ID NO: 293), X-EDPEY*F-C(Sx)-MK-Z (SEQ ID NO: 294), X-EDPEY*F-C(Sx)-MP-Z (SEQ ID NO: 295), X-EDPEY*F-C(Sx)-VG-Z (SEQ ID NO: 296), X-EDPEY*F-C(Sx)-VK-Z (SEQ ID NO: 297), X-EDPEY*F-C(Sx)-VP-Z (SEQ ID NO: 298), X-EDPEY*I-C(Sx)-FG-Z (SEQ ID NO: 299), X-EDPEY*I-C(Sx)-FK-Z (SEQ ID NO: 300), X-EDPEY*I-C(Sx)-FP-Z (SEQ ID NO: 301), X-EDPEY*I-C(Sx)-IG-Z (SEQ ID NO: 302), X-EDPEY*I-C(Sx)-IK-Z (SEQ ID NO: 303), X-EDPEY*I-C(Sx)-IP-Z (SEQ ID NO: 304), X-EDPEY*I-C(Sx)-MG-Z (SEQ ID NO: 305), X-EDPEY*I-C(Sx)-MK-Z (SEQ ID NO: 306), X-EDPEY*I-C(Sx)-MP-Z (SEQ ID NO: 307), X-EDPEY*I-C(Sx)-VG-Z (SEQ ID NO: 308), X-EDPEY*I-C(Sx)-VK-Z (SEQ ID NO: 309), X-EDPEY*I-C(Sx)-VP-Z (SEQ ID NO: 310), X-EDPEY*V-C(Sx)-FG-Z (SEQ ID NO: 311), X-EDPEY*V-C(Sx)-FK-Z (SEQ ID NO: 312), X-EDPEY*V-C(Sx)-FP-Z (SEQ ID NO: 313), X-EDPEY*V-C(Sx)-IG-Z (SEQ ID NO: 314), X-EDPEY*V-C(Sx)-IK-Z (SEQ ID NO: 315), X-EDPEY*V-C(Sx)-IP-Z (SEQ ID NO: 316), X-EDPEY*V-C(Sx)-MG-Z (SEQ ID NO: 317), X-EDPEY*V-C(Sx)-MK-Z (SEQ ID NO: 318), X-EDPEY*V-C(Sx)-MP-Z (SEQ ID NO: 319), X-EDPEY*V-C(Sx)-VG-Z (SEQ ID NO: 320), X-EDPEY*V-C(Sx)-VK-Z (SEQ ID NO: 321), and X-EDPEY*V-C(Sx)-VP-Z (SEQ ID NO: 322); wherein X— is H or acetyl; and Z is OH or NH2.

In some embodiments, the metal-binding compound of the present invention is selected from the group consisting of: X-EDPIY*F-C(Sx)-FG-Z (SEQ ID NO: 323), X-EDPIY*F-C(Sx)-FK-Z (SEQ ID NO: 324), X-EDPIY*F-C(Sx)-FP-Z (SEQ ID NO: 325), X-EDPIY*F-C(Sx)-IG-Z (SEQ ID NO: 326), X-EDPIY*F-C(Sx)-IK-Z (SEQ ID NO: 327), X-EDPIY*F-C(Sx)-IP-Z (SEQ ID NO: 328), X-EDPIY*F-C(Sx)-MG-Z (SEQ ID NO: 329), X-EDPIY*F-C(Sx)-MK-Z (SEQ ID NO: 330), X-EDPIY*F-C(Sx)-MP-Z (SEQ ID NO: 331), X-EDPIY*F-C(Sx)-VG-Z (SEQ ID NO: 332), X-EDPIY*F-C(Sx)-VK-Z (SEQ ID NO: 333), X-EDPIY*F-C(Sx)-VP-Z (SEQ ID NO: 334), X-EDPIY*I-C(Sx)-FG-Z (SEQ ID NO: 335), X-EDPIY*I-C(Sx)-FK-Z (SEQ ID NO: 336), X-EDPIY*I-C(Sx)-FP-Z (SEQ ID NO: 337), X-EDPIY*I-C(Sx)-IG-Z (SEQ ID NO: 338), X-EDPIY*I-C(Sx)-IK-Z (SEQ ID NO: 339), X-EDPIY*I-C(Sx)-IP-Z (SEQ ID NO: 340), X-EDPIY*I-C(Sx)-MG-Z (SEQ ID NO: 341), X-EDPIY*I-C(Sx)-MK-Z (SEQ ID NO: 342), X-EDPIY*I-C(Sx)-MP-Z (SEQ ID NO: 343), X-EDPIY*I-C(Sx)-VG-Z (SEQ ID NO: 344), X-EDPIY*I-C(Sx)-VK-Z (SEQ ID NO: 345), X-EDPIY*V-C(Sx)-FG-Z (SEQ ID NO: 347), X-EDPIY*V-C(Sx)-FK-Z (SEQ ID NO: 348), X-EDPIY*V-C(Sx)-FP-Z (SEQ ID NO: 349), X-EDPIY*V-C(Sx)-IG-Z (SEQ ID NO: 350), X-EDPIY*V-C(Sx)-IK-Z (SEQ ID NO: 351), X-EDPIY*V-C(Sx)-IP-Z (SEQ ID NO: 352), X-EDPIY*V-C(Sx)-MG-Z (SEQ ID NO: 353), X-EDPIY*V-C(Sx)-MK-Z (SEQ ID NO: 354), X-EDPIY*V-C(Sx)-MP-Z (SEQ ID NO: 355), X-EDPIY*V-C(Sx)-VG-Z (SEQ ID NO: 356), X-EDPIY*V-C(Sx)-VK-Z (SEQ ID NO: 357), X-EDPIY*V-C(Sx)-VP-Z (SEQ ID NO: 358), X-EDSFLQRY*S-C(Sx)-DPTGA-Z (SEQ ID NO: 940), X-EEEEEY*I-C(Sx)-Z (SEQ ID NO: 4873), X-EEEEY*[Nle]-C(Sx)-VG-Z (SEQ ID NO: 4874), X-EEEEY*[Nle]Q[dP]-C(Sx)-G-Z (SEQ ID NO: 4875), X-EEEEY*[Nle]Q-C(Sx)-VG-Z (SEQ ID NO: 4876), X-EEEEY*A-C(Sx)-VG-Z (SEQ ID NO: 4877), X-EEEEY*AQ[dP]-C(Sx)-G-Z (SEQ ID NO: 4878), X-EEEEY*AQ-C(Sx)-VG-Z (SEQ ID NO: 4879), X-EEEEY*D-C(Sx)-VG-Z (SEQ ID NO: 4880), X-EEEEY*DQ[dP]-C(Sx)-G-Z (SEQ ID NO: 4881), X-EEEEY*DQ-C(Sx)-VG-Z (SEQ ID NO: 4882), X-EEEEY*E-C(Sx)-VG-Z (SEQ ID NO: 4883), X-EEEEY*EQ[dP]-C(Sx)-G-Z (SEQ ID NO: 4884), X-EEEEY*EQ-C(Sx)-VG-Z (SEQ ID NO: 4885), X-EEEEY*F-C(Sx)-VG-Z (SEQ ID NO: 4886), X-EEEEY*FQ[dP]-C(Sx)-G-Z (SEQ ID NO: 4887), X-EEEEY*FQ-C(Sx)-VG-Z (SEQ ID NO: 4888), X-EEEEY*G-C(Sx)-VG-Z (SEQ ID NO: 4889), X-EEEEY*GG[dP]-C(Sx)-G-Z (SEQ ID NO: 4890), X-EEEEY*GG-C(Sx)-VG-Z (SEQ ID NO: 4891), X-EEEEY*GGG-C(Sx)-G-Z (SEQ ID NO: 4892), X-EEEEY*GQ[dP]-C(Sx)-G-Z (SEQ ID NO: 4893), X-EEEEY*GQ-C(Sx)-VG-Z (SEQ ID NO: 4894), X-EEEEY*H-C(Sx)-VG-Z (SEQ ID NO: 4895), X-EEEEY*HQ[dP]-C(Sx)-G-Z (SEQ ID NO: 4896), X-EEEEY*HQ-C(Sx)-VG-Z (SEQ ID NO: 4897), X-EEEEY*I[Nle][dP]-C(Sx)-G-Z (SEQ ID NO: 4898), X-EEEEY*I[Nle]-C(Sx)-VG-Z (SEQ ID NO: 4899), X-EEEEY*IA[dP]-C(Sx)-G-Z (SEQ ID NO: 4900), X-EEEEY*IA-C(Sx)-VG-Z (SEQ ID NO: 4901), X-EEEEY*I-C(Sx)-EV-Z (SEQ ID NO: 4902), X-EEEEY*I-C(Sx)-IV-Z (SEQ ID NO: 1), X-EEEEY*ID[dP]-C(Sx)-G-Z (SEQ ID NO: 4903), X-EEEEY*ID-C(Sx)-VG-Z (SEQ ID NO: 4904), X-EEEEY*IE[dP]-C(Sx)-G-Z (SEQ ID NO: 4905), X-EEEEY*IE-C(Sx)-VG-Z (SEQ ID NO: 4906), X-EEEEY*IF[dP]-C(Sx)-G-Z (SEQ ID NO: 4907), X-EEEEY*IF-C(Sx)-VG-Z (SEQ ID NO: 4908), X-EEEEY*IG[dP]-C(Sx)-G-Z (SEQ ID NO: 4909), X-EEEEY*IG-C(Sx)-VG-Z (SEQ ID NO: 4910), X-EEEEY*IH[dP]-C(Sx)-G-Z (SEQ ID NO: 4911), X-EEEEY*IH-C(Sx)-VG-Z (SEQ ID NO: 4912), X-EEEEY*II[dP]-C(Sx)-G-Z (SEQ ID NO: 4913), X-EEEEY*II-C(Sx)-VG-Z (SEQ ID NO: 4914), X-EEEEY*IK[dP]-C(Sx)-G-Z (SEQ ID NO: 4915), X-EEEEY*IK-C(Sx)-VG-Z (SEQ ID NO: 4916), X-EEEEY*IL[dP]-C(Sx)-G-Z (SEQ ID NO: 4917), X-EEEEY*IL-C(Sx)-VG-Z (SEQ ID NO: 4918), X-EEEEY*IN[dP]-C(Sx)-G-Z (SEQ ID NO: 4919), X-EEEEY*IN-C(Sx)-VG-Z (SEQ ID NO: 4920), X-EEEEY*IP[dP]-C(Sx)-G-Z (SEQ ID NO: 4921), X-EEEEY*IP-C(Sx)-VG-Z (SEQ ID NO: 4922), X-EEEEY*IQ[dP]-C(Sx)-G-Z (SEQ ID NO: 4923), X-EEEEY*IQ[Nle]-C(Sx)-G-Z (SEQ ID NO: 4924), X-EEEEY*IQA-C(Sx)-G-Z (SEQ ID NO: 4925), X-EEEEY*IQ-C(Sx)-VG-Z (SEQ ID NO: 4926), X-EEEEY*IQD-C(Sx)-G-Z (SEQ ID NO: 4927), X-EEEEY*IQE-C(Sx)-G-Z (SEQ ID NO: 4928), X-EEEEY*IQF-C(Sx)-G-Z (SEQ ID NO: 4929), X-EEEEY*IQG-C(Sx)-G-Z (SEQ ID NO: 4930), X-EEEEY*IQH-C(Sx)-G-Z (SEQ ID NO: 4931), X-EEEEY*IQI-C(Sx)-G-Z (SEQ ID NO: 4932), X-EEEEY*IQK-C(Sx)-G-Z (SEQ ID NO: 4933), X-EEEEY*IQL-C(Sx)-G-Z (SEQ ID NO: 4934), X-EEEEY*IQN-C(Sx)-G-Z (SEQ ID NO: 4935), X-EEEEY*IQP-C(Sx)-G-Z (SEQ ID NO: 4936), X-EEEEY*IQQ-C(Sx)-G-Z (SEQ ID NO: 4937), X-EEEEY*IQR-C(Sx)-G-Z (SEQ ID NO: 4938), X-EEEEY*IQV-C(Sx)-G-Z (SEQ ID NO: 4939); and X-EEEEY*IQW-C(Sx)-G-Z (SEQ ID NO: 4940); wherein X— is H or acetyl; and Z is OH or NH2.

In some embodiments, the metal-binding compound of the present invention is selected from the group consisting of: X-EEEEY*IR[dP]-C(Sx)-G-Z (SEQ ID NO: 4941), X-EEEEY*IR-C(Sx)-VG-Z (SEQ ID NO: 4942), X-EEEEY*IV[dP]-C(Sx)-G-Z (SEQ ID NO: 4943), X-EEEEY*IV-C(Sx)-VG-Z (SEQ ID NO: 4944), X-EEEEY*IW[dP]-C(Sx)-G-Z (SEQ ID NO: 4945), X-EEEEY*IW-C(Sx)-VG-Z (SEQ ID NO: 4946), X-EEEEY*K-C(Sx)-VG-Z (SEQ ID NO: 4947), X-EEEEY*KQ[dP]-C(Sx)-G-Z (SEQ ID NO: 4948), X-EEEEY*KQ-C(Sx)-VG-Z (SEQ ID NO: 4949), X-EEEEY*L-C(Sx)-VG-Z (SEQ ID NO: 4950), X-EEEEY*LQ[dP]-C(Sx)-G-Z (SEQ ID NO: 4951), X-EEEEY*LQ-C(Sx)-VG-Z (SEQ ID NO: 4952), X-EEEEY*N-C(Sx)-VG-Z (SEQ ID NO: 4953), X-EEEEY*NQ[dP]-C(Sx)-G-Z (SEQ ID NO: 4954), X-EEEEY*NQ-C(Sx)-VG-Z (SEQ ID NO: 4955), X-EEEEY*P-C(Sx)-VG-Z (SEQ ID NO: 4956), X-EEEEY*PQ[dP]-C(Sx)-G-Z (SEQ ID NO: 4957), X-EEEEY*PQ-C(Sx)-VG-Z (SEQ ID NO: 4958), X-EEEEY*Q-C(Sx)-VG-Z (SEQ ID NO: 4959), X-EEEEY*QQ[dP]-C(Sx)-G-Z (SEQ ID NO: 4960), X-EEEEY*QQ-C(Sx)-VG-Z (SEQ ID NO: 4961), X-EEEEY*R-C(Sx)-VG-Z (SEQ ID NO: 4962), X-EEEEY*RQ[dP]-C(Sx)-G-Z (SEQ ID NO: 4963), X-EEEEY*RQ-C(Sx)-VG-Z (SEQ ID NO: 4964), X-EEEEY*V-C(Sx)-VG-Z (SEQ ID NO: 4965), X-EEEEY*VQ[dP]-C(Sx)-G-Z (SEQ ID NO: 4966), X-EEEEY*VQ-C(Sx)-VG-Z (SEQ ID NO: 4967), X-EEEEY*W-C(Sx)-VG-Z (SEQ ID NO: 4968), X-EEEEY*WQ[dP]-C(Sx)-G-Z (SEQ ID NO: 4969), X-EEEEY*WQ-C(Sx)-VG-Z (SEQ ID NO: 4970), X-EEEIY*F-C(Sx)-FWG-Z (SEQ ID NO: 922), X-EEEY*I-C(Sx)-[Nle]V-Z (SEQ ID NO: 4971), X-EEEY*I-C(Sx)-AV-Z (SEQ ID NO: 4972), X-EEEY*I-C(Sx)-DV-Z (SEQ ID NO: 4973), X-EEEY*I-C(Sx)-FV-Z (SEQ ID NO: 4974), X-EEEY*I-C(Sx)-GV-Z (SEQ ID NO: 4975), X-EEEY*I-C(Sx)-HV-Z (SEQ ID NO: 4976), X-EEEY*I-C(Sx)-I[Nle]-Z (SEQ ID NO: 4977), X-EEEY*I-C(Sx)-IA-Z (SEQ ID NO: 4978), X-EEEY*I-C(Sx)-ID-Z (SEQ ID NO: 4979), X-EEEY*I-C(Sx)-IE-Z (SEQ ID NO: 4980), X-EEEY*I-C(Sx)-IF-Z (SEQ ID NO: 4981), X-EEEY*I-C(Sx)-IG-Z (SEQ ID NO: 4982), X-EEEY*I-C(Sx)-IH-Z (SEQ ID NO: 4983), X-EEEY*I-C(Sx)-II-Z (SEQ ID NO: 4984), X-EEEY*I-C(Sx)-IK-Z (SEQ ID NO: 4985), X-EEEY*I-C(Sx)-IL-Z (SEQ ID NO: 4986), X-EEEY*I-C(Sx)-IM-Z (SEQ ID NO: 4987), X-EEEY*I-C(Sx)-IN-Z (SEQ ID NO: 4988), X-EEEY*I-C(Sx)-IP-Z (SEQ ID NO: 4989), X-EEEY*I-C(Sx)-IQ-Z (SEQ ID NO: 4990), X-EEEY*I-C(Sx)-IR-Z (SEQ ID NO: 4991), X-EEEY*I-C(Sx)-IW-Z (SEQ ID NO: 4992), X-EEEY*I-C(Sx)-KV-Z (SEQ ID NO: 4993), X-EEEY*I-C(Sx)-LV-Z (SEQ ID NO: 4994), X-EEEY*I-C(Sx)-MV-Z (SEQ ID NO: 4995), X-EEEY*I-C(Sx)-NV-Z (SEQ ID NO: 4996), X-EEEY*I-C(Sx)-PV-Z (SEQ ID NO: 4997), X-EEEY*I-C(Sx)-QV-Z (SEQ ID NO: 4998), X-EEEY*I-C(Sx)-RV-Z (SEQ ID NO: 4999), X-EEEY*I-C(Sx)-VV-Z (SEQ ID NO: 5000), X-EEEY*I-C(Sx)-WV-Z (SEQ ID NO: 5001), X-EEPDY*E-C(Sx)-QP-Z (SEQ ID NO: 5002), X-EEPDY*F-C(Sx)-FG-Z (SEQ ID NO: 359), X-EEPDY*F-C(Sx)-FK-Z (SEQ ID NO: 360), X-EEPDY*F-C(Sx)-FP-Z (SEQ ID NO: 361), X-EEPDY*F-C(Sx)-IG-Z (SEQ ID NO: 362), X-EEPDY*F-C(Sx)-IK-Z (SEQ ID NO: 363), X-EEPDY*F-C(Sx)-IP-Z (SEQ ID NO: 364), X-EEPDY*F-C(Sx)-MG-Z (SEQ ID NO: 365), X-EEPDY*F-C(Sx)-MK-Z (SEQ ID NO: 366), X-EEPDY*F-C(Sx)-MP-Z (SEQ ID NO: 367), X-EEPDY*F-C(Sx)-VG-Z (SEQ ID NO: 368), X-EEPDY*F-C(Sx)-VK-Z (SEQ ID NO: 369), X-EEPDY*F-C(Sx)-VP-Z (SEQ ID NO: 370), X-EEPDY*I-C(Sx)-FG-Z (SEQ ID NO: 371), X-EEPDY*I-C(Sx)-FK-Z (SEQ ID NO: 372), X-EEPDY*I-C(Sx)-FP-Z (SEQ ID NO: 373), X-EEPDY*I-C(Sx)-IG-Z (SEQ ID NO: 374), X-EEPDY*I-C(Sx)-IK-Z (SEQ ID NO: 375), X-EEPDY*I-C(Sx)-IP-Z (SEQ ID NO: 376), X-EEPDY*I-C(Sx)-MG-Z (SEQ ID NO: 377), X-EEPDY*I-C(Sx)-MK-Z (SEQ ID NO: 378), X-EEPDY*I-C(Sx)-MP-Z (SEQ ID NO: 379), X-EEPDY*I-C(Sx)-VG-Z (SEQ ID NO: 380), X-EEPDY*I-C(Sx)-VK-Z (SEQ ID NO: 381), X-EEPDY*V-C(Sx)-FG-Z (SEQ ID NO: 383), X-EEPDY*V-C(Sx)-FK-Z (SEQ ID NO: 384), X-EEPDY*V-C(Sx)-FP-Z (SEQ ID NO: 385), X-EEPDY*V-C(Sx)-IG-Z (SEQ ID NO: 386), X-EEPDY*V-C(Sx)-IK-Z (SEQ ID NO: 387), X-EEPDY*V-C(Sx)-IP-Z (SEQ ID NO: 388), X-EEPDY*V-C(Sx)-MG-Z (SEQ ID NO: 389), X-EEPDY*V-C(Sx)-MK-Z (SEQ ID NO: 390), X-EEPDY*V-C(Sx)-MP-Z (SEQ ID NO: 391), X-EEPDY*V-C(Sx)-VG-Z (SEQ ID NO: 392), X-EEPDY*V-C(Sx)-VK-Z (SEQ ID NO: 393), X-EEPDY*V-C(Sx)-VP-Z (SEQ ID NO: 394), X-EEPEY*F-C(Sx)-FG-Z (SEQ ID NO: 395), X-EEPEY*F-C(Sx)-FK-Z (SEQ ID NO: 396), X-EEPEY*F-C(Sx)-FP-Z (SEQ ID NO: 397), X-EEPEY*F-C(Sx)-IG-Z (SEQ ID NO: 398), X-EEPEY*F-C(Sx)-IK-Z (SEQ ID NO: 399), X-EEPEY*F-C(Sx)-IP-Z (SEQ ID NO: 400), X-EEPEY*F-C(Sx)-MG-Z (SEQ ID NO: 401), X-EEPEY*F-C(Sx)-MK-Z (SEQ ID NO: 402), X-EEPEY*F-C(Sx)-MP-Z (SEQ ID NO: 403), X-EEPEY*F-C(Sx)-VG-Z (SEQ ID NO: 404), X-EEPEY*F-C(Sx)-VK-Z (SEQ ID NO: 405), and X-EEPEY*F-C(Sx)-VP-Z (SEQ ID NO: 406); wherein X— is H or acetyl; and Z is OH or NH2.

In some embodiments, the metal-binding compound of the present invention is selected from the group consisting of: X-EEPEY*I-C(Sx)-FG-Z (SEQ ID NO: 407), X-EEPEY*I-C(Sx)-FK-Z (SEQ ID NO: 408), X-EEPEY*I-C(Sx)-FP-Z (SEQ ID NO: 409), X-EEPEY*I-C(Sx)-IG-Z (SEQ ID NO: 410), X-EEPEY*I-C(Sx)-IK-Z (SEQ ID NO: 411), X-EEPEY*I-C(Sx)-IP-Z (SEQ ID NO: 412), X-EEPEY*I-C(Sx)-MG-Z (SEQ ID NO: 413), X-EEPEY*I-C(Sx)-MK-Z (SEQ ID NO: 414), X-EEPEY*I-C(Sx)-MP-Z (SEQ ID NO: 415), X-EEPEY*I-C(Sx)-VG-Z (SEQ ID NO: 416), X-EEPEY*I-C(Sx)-VK-Z (SEQ ID NO: 417), X-EEPEY*V-C(Sx)-FG-Z (SEQ ID NO: 419), X-EEPEY*V-C(Sx)-FK-Z (SEQ ID NO: 420), X-EEPEY*V-C(Sx)-FP-Z (SEQ ID NO: 421), X-EEPEY*V-C(Sx)-IG-Z (SEQ ID NO: 422), X-EEPEY*V-C(Sx)-IK-Z (SEQ ID NO: 423), X-EEPEY*V-C(Sx)-IP-Z (SEQ ID NO: 424), X-EEPEY*V-C(Sx)-MG-Z (SEQ ID NO: 425), X-EEPEY*V-C(Sx)-MK-Z (SEQ ID NO: 426), X-EEPEY*V-C(Sx)-MP-Z (SEQ ID NO: 427), X-EEPEY*V-C(Sx)-VG-Z (SEQ ID NO: 428), X-EEPEY*V-C(Sx)-VK-Z (SEQ ID NO: 429), and X-EEPEY*V-C(Sx)-VP-Z (SEQ ID NO: 430), X-EEPIY*F-C(Sx)-FG-Z (SEQ ID NO: 458), X-EEPIY*F-C(Sx)-FK-Z (SEQ ID NO: 459), X-EEPIY*F-C(Sx)-FP-Z (SEQ ID NO: 461), X-EEPIY*F-C(Sx)-IG-Z (SEQ ID NO: 462), X-EEPIY*F-C(Sx)-IK-Z (SEQ ID NO: 463), X-EEPIY*F-C(Sx)-IP-Z (SEQ ID NO: 465), X-EEPIY*F-C(Sx)-MG-Z (SEQ ID NO: 469), X-EEPIY*F-C(Sx)-MK-Z (SEQ ID NO: 470), X-EEPIY*F-C(Sx)-MP-Z (SEQ ID NO: 472), X-EEPIY*F-C(Sx)-VG-Z (SEQ ID NO: 473), X-EEPIY*F-C(Sx)-VK-Z (SEQ ID NO: 474), X-EEPIY*I-C(Sx)-FG-Z (SEQ ID NO: 483), X-EEPIY*I-C(Sx)-FK-Z (SEQ ID NO: 484), X-EEPIY*I-C(Sx)-IG-Z (SEQ ID NO: 487), X-EEPIY*I-C(Sx)-IK-Z (SEQ ID NO: 488), X-EEPIY*I-C(Sx)-MG-Z (SEQ ID NO: 494), X-EEPIY*I-C(Sx)-MK-Z (SEQ ID NO: 495), X-EEPIY*V-C(Sx)-FG-Z (SEQ ID NO: 531), X-EEPIY*V-C(Sx)-FK-Z (SEQ ID NO: 532), X-EEPIY*V-C(Sx)-FP-Z (SEQ ID NO: 534), X-EEPIY*V-C(Sx)-IG-Z (SEQ ID NO: 535), X-EEPIY*V-C(Sx)-IK-Z (SEQ ID NO: 536), X-EEPIY*V-C(Sx)-IP-Z (SEQ ID NO: 538), X-EEPIY*V-C(Sx)-MG-Z (SEQ ID NO: 542), X-EEPIY*V-C(Sx)-MK-Z (SEQ ID NO: 543), X-EEPIY*V-C(Sx)-MP-Z (SEQ ID NO: 545), X-EEPIY*V-C(Sx)-VG-Z (SEQ ID NO: 546), X-EEPIY*V-C(Sx)-VK-Z (SEQ ID NO: 547), X-EEPPD-C(Sx)-QY*[pY]NDFPGK-Z (SEQ ID NO: 974), X-EEPPD-C(Sx)-QY*YNDFPGK-Z (SEQ ID NO: 973), X-EGRNPGFY*V-C(Sx)-ANPMP-Z (SEQ ID NO: 967), X-EGSFESRY*Q-C(Sx)-PFEDF-Z (SEQ ID NO: 965), X-EPIQEANY*V-C(Sx)-MTPGT-Z (SEQ ID NO: 955), X-EPPDHQYY*N-C(Sx)-FPGKE-Z (SEQ ID NO: 975), X-EQEDEPE-C(Sx)-DY*EEVLE-Z (SEQ ID NO: 5003), X-EQEDEPE-C(Sx)-DY*FEWLE-Z (SEQ ID NO: 5004), X-EQEDEPEGDY*E-C(Sx)-VLE-Z (SEQ ID NO: 5005), X-EQEDEPEGDY*F-C(Sx)-WLE-Z (SEQ ID NO: 5006), X-EVSETDDY*A-C(Sx)-IIDEE-Z (SEQ ID NO: 5007), X-FAGKE-C(Sx)-TY*[pY]QGRHLG-Z (SEQ ID NO: 980), X-FAGKE-C(Sx)-TY*YQGRHLG-Z (SEQ ID NO: 979), X-FD[Nle]DR-C(Sx)-IY*RMSFFRK-Z (SEQ ID NO: 5008), X-FD[Nle]DRDIY*R-C(Sx)-SFFRK-Z (SEQ ID NO: 5009), X-FDKDGNGY*I-C(Sx)-AAELR-Z (SEQ ID NO: 930), X-FDMDR-C(Sx)-IY*RMSFFRK-Z (SEQ ID NO: 5010), X-FDMDRDIY*R-C(Sx)-SFFRK-Z (SEQ ID NO: 5011), X-FEEEEY*I-C(Sx)-Z (SEQ ID NO: 5012), X-FEEEY*I-C(Sx)-IV-Z (SEQ ID NO: 5013), X-FTDRL-C(Sx)-QY*ISTRGLG-Z (SEQ ID NO: 919), X-FTDRLQQY*I-C(Sx)-TRGLG-Z (SEQ ID NO: 920), and X-FVSETDDY*A-C(Sx)-IIDEE-Z (SEQ ID NO: 5014); wherein X is H or acetyl; and Z is OH or NH2.

In some embodiments, the metal-binding compound of the present invention is selected from the group consisting of: X-FYEEIKKY*E-C(Sx)-LETEE-Z (SEQ ID NO: 928), X-GAEDPEY*I-C(Sx)-IPAKKKG-Z (SEQ ID NO: 5015), X-GAEEPDY*I-C(Sx)-FGAKKKG-Z (SEQ ID NO: 5016), X-GAEEPEY*I-C(Sx)-FGAKKKG-Z (SEQ ID NO: 5017), X-GAEEPEY*I-C(Sx)-VKAKKKG-Z (SEQ ID NO: 5018), X-GAEEPIY*I-C(Sx)-[Nle]PAKKKG-Z (SEQ ID NO: 5019), X-GAEEPIY*I-C(Sx)-FGAKKKG-Z (SEQ ID NO: 5020), X-GAEEPIY*V-C(Sx)-FGAKKKG-Z (SEQ ID NO: 5021), X-GAEEPIY*V-C(Sx)-VKAKKKG-Z (SEQ ID NO: 5022), X-GAEEPIY*V-C(Sx)-VPAKKKG-Z (SEQ ID NO: 5023), X-GAGEE-C(Sx)-DY*[pY][pY]IWAGKKKK-Z (SEQ ID NO: 914), X-GC(Xx)-NY*RGYSLGNWV-Z (SEQ ID NO: 921), X-GDGSD-C(Sx)-PY*[pY]NSIPSK-Z (SEQ ID NO: 977), X-GDGSD-C(Sx)-PY*YNSIPSK-Z (SEQ ID NO: 976), X-GEEEEY*I-C(Sx)-Z (SEQ ID NO: 5024), X-GEEEY*I-C(Sx)-IV-Z (SEQ ID NO: 5025), X-GGPEPGPY*A-C(Sx)-PSVNT-Z (SEQ ID NO: 936), X-GLPWWR-C(Sx)-YT*WVVERDVNTKQR-Z (SEQ ID NO: 5026), X-GSNQEEAY*V-C(Sx)-[Nle]SSFY-Z (SEQ ID NO: 5027), X-GVSETDDY*A-C(Sx)-IIDEE-Z (SEQ ID NO: 5028), X-HDLGEDIY*D-C(Sx)-VP-Z (SEQ ID NO: 985), X-HEEEEY*I-C(Sx)-Z (SEQ ID NO: 5029), X-HEEEY*I-C(Sx)-IV-Z (SEQ ID NO: 5030), X-HRIDG-C(Sx)-TY*VIKRVKY*-Z (SEQ ID NO: 5031), X-HRIDGKTY*V-C(Sx)-KRVKY-Z (SEQ ID NO: 5032), X-HVNATY*V-C(Sx)-VKSVAP-Z (SEQ ID NO: 5033), X-HVSETDDY*A-C(Sx)-IIDEE-Z (SEQ ID NO: 5034), X-IEEEEY*I-C(Sx)-Z (SEQ ID NO: 5035), X-IEEEY*I-C(Sx)-IV-Z (SEQ ID NO: 5036), X-IENEEQEY*V-C(Sx)-TVKSS-Z (SEQ ID NO: 925), X-IGTAE-C(Sx)-DY*GALYEGR-Z (SEQ ID NO: 966), X-ISLDNPDY*Q-C(Sx)-DFFPK-Z (SEQ ID NO: 944), X-IVSETDDY*A-C(Sx)-IIDEE-Z (SEQ ID NO: 5037), X-KAEEEEY*I-C(Sx)-LVA-Z (SEQ ID NO: 996), X-KEEEEY*I-C(Sx)-Z (SEQ ID NO: 5038), X-KEEEY*I-C(Sx)-IV-Z (SEQ ID NO: 5039), X-KEVHKSGY*L-C(Sx)-SERLI-Z (SEQ ID NO: 950), X-KGDKQVEY*L-C(Sx)-LDLDS-Z (SEQ ID NO: 957), X-KKAEEEEY*I-C(Sx)-LVA-Z (SEQ ID NO: 995), X-KKEEEIY*F-C(Sx)-FWG-Z (SEQ ID NO: 923), X-KKGE-C(Sx)-IY*AAPFA-Z (SEQ ID NO: 5040), X-KKKAEEEEY*I-C(Sx)-LVA-Z (SEQ ID NO: 994), X-KKKAEEEEY*I-C(Sx)-LV-Z (SEQ ID NO: 5041), X-KKKAEEEEY*I-C(Sx)-L-Z (SEQ ID NO: 910), X-KKKAEEEEY*I-C(Sx)-Z (SEQ ID NO: 911), X-KKKAEPLPPSY*V-C(Sx)-AS-Z (SEQ ID NO: 5042), X-KKKDEQIY*W-C(Sx)-IA-Z (SEQ ID NO: 997), X-KKKDNVLINTY*S-C(Sx)-VLKIS-Z (SEQ ID NO: 5043), X-KKKEEEIY*F-C(Sx)-FWG-Z (SEQ ID NO: 917), X-KKKKE-C(Sx)-IY*FFFG-Z (SEQ ID NO: 5044), X-KKKKEEEIY*F-C(Sx)-FWG-Z (SEQ ID NO: 918), X-KKKLSRSLY*F-C(Sx)-PLLH-Z (SEQ ID NO: 5045), X-KKKLTIDRY*L-C(Sx)-IVHAV-Z (SEQ ID NO: 5046), and X-KKKNHIGHTGY*L-C(Sx)-TVTVS-Z (SEQ ID NO: 5047); wherein X— is H or acetyl; and Z is OH or NH2.

In some embodiments, the metal-binding compound of the present invention is selected from the group consisting of: X-KKKQAATGFGSP-C(Sx)-QY*SAD-Z (SEQ ID NO: 5048), X-KKKRAHEEIY*F-C(Sx)-FWG-Z (SEQ ID NO: 916), X-KKKSNPALLSSP-C(Sx)-YY*SAA-Z (SEQ ID NO: 5049), X-KKKTSF[Nle][Nle][pT]PY*V-C(Sx)-TRY-Z (SEQ ID NO: 5050), X-KKSR-C(Sx)-DY*[Nle]T[Nle]QIG-Z (SEQ ID NO: 5051), X-KKTNSSEGLSMGNY*I-C(Sx)-LINR-Z (SEQ ID NO: 5052), X-KVSETDDY*A-C(Sx)-IIDEE-Z (SEQ ID NO: 5053), X-LEEEEY*I-C(Sx)-Z (SEQ ID NO: 5054), X-LEEEY*I-C(Sx)-IV-Z (SEQ ID NO: 5055), X-LHQEDNDY*I-C(Sx)-ASLIK-Z (SEQ ID NO: 969), X-LHQEDNDY*IN-C(Sx)-SLIK-Z (SEQ ID NO: 970), X-LMLRLQDY*E-C(Sx)-KTKKA-Z (SEQ ID NO: 951), X-LSRDLIMK-C(Sx)-DY*ELVSTKPTR-Z (SEQ ID NO: 5056), X-LSRDLIMKEDY*E-C(Sx)-VSTKPTR-Z (SEQ ID NO: 5057), X-LSRDLIMKEDY*E-C(Sx)-VSTK-Z (SEQ ID NO: 5058), X-LVSETDDY*A-C(Sx)-IIDEE-Z (SEQ ID NO: 5059), X-MAEEKKAY*K-C(Sx)-AKERE-Z (SEQ ID NO: 932), X-MEEEEY*I-C(Sx)-Z (SEQ ID NO: 5060), X-MEEEY*I-C(Sx)-IV-Z (SEQ ID NO: 5061), X-MK-C(Sx)-DY*ELVSTKPTRTSKVR-Z (SEQ ID NO: 5062), X-MQDNS-C(Sx)-TY*GKIWEGS-Z (SEQ ID NO: 963), X-MRHVSISY*D-C(Sx)-PPTPG-Z (SEQ ID NO: 953), X-NEEEEY*I-C(Sx)-Z (SEQ ID NO: 5063), X-NEEEY*I-C(Sx)-IV-Z (SEQ ID NO: 5064), X-NFANFSAY*P-C(Sx)-EEDMI-Z (SEQ ID NO: 949), X-NHIG-C(Sx)-TGY*LNTVTVSAKKK-Z (SEQ ID NO: 5065), X-NKVDD-C(Sx)-NY*AIKRIRL-Z (SEQ ID NO: 5066), X-NKVDDCNY*A-C(Sx)-KRIRL-Z (SEQ ID NO: 5067), X-NVSETDDY*A-C(Sx)-IIDEE-Z (SEQ ID NO: 5068), X-PAFDNLYY*W-C(Sx)-QDPPE-Z (SEQ ID NO: 990), X-PEEEEY*I-C(Sx)-Z (SEQ ID NO: 5069), X-PEEEY*I-C(Sx)-IV-Z (SEQ ID NO: 5070), X-PTAENPEY*L-C(Sx)-LDVPV-Z (SEQ ID NO: 991), X-PTGEAPTY*V-C(Sx)-TQQIP-Z (SEQ ID NO: 983), X-PVPEY*I-C(Sx)-QSV-Z (SEQ ID NO: 941), X-PVSETDDY*A-C(Sx)-IIDEE-Z (SEQ ID NO: 5071), X-QALDNPEY*H-C(Sx)-ASNGP-Z (SEQ ID NO: 992), X-QEEEEY*I-C(Sx)-Z (SEQ ID NO: 5072), X-QEEEY*I-C(Sx)-IV-Z (SEQ ID NO: 5073), X-QLAAK-C(Sx)-AY*LQILSEE-Z (SEQ ID NO: 5074), X-QLAAKLAY*L-C(Sx)-ILSEE-Z (SEQ ID NO: 5075), X-QLTFALRY*L-C(Sx)-FFTKA-Z (SEQ ID NO: 961), X-QVSETDDY*A-C(Sx)-IIDEE-Z (SEQ ID NO: 5076), X-RAEEEEY*I-C(Sx)-LVA-Z (SEQ ID NO: 909), X-RAHEEIY*F-C(Sx)-FWG-Z (SEQ ID NO: 924), X-R-C(Sx)-DY*[Nle]TMQIGKK-Z (SEQ ID NO: 5077), X-REEEEY*I-C(Sx)-Z (SEQ ID NO: 5078), X-REEEY*I-C(Sx)-IV-Z (SEQ ID NO: 5079), X-RLDGE-C(Sx)-IY*IRHSNLM-Z (SEQ ID NO: 948), X-RNEEENIY*S-C(Sx)-PHDST-Z (SEQ ID NO: 959), X-RNEGVATY*A-C(Sx)-AVLFR-Z (SEQ ID NO: 937), X-RQGSSDIY*S-C(Sx)-PEGKL-Z (SEQ ID NO: 982), X-RRAEEEEY*I-C(Sx)-LVA-Z (SEQ ID NO: 908), X-RRHY*Y-C(Sx)-DTHTNTYYLRTFGHNTRR-Z (SEQ ID NO: 5080), X-RRHYYYDTHTNTY*Y-C(Sx)-RTFGHNTRR-Z (SEQ ID NO: 5081), X-RRRAEEEEY*I-C(Sx)-LVA-Z (SEQ ID NO: 993), X-RVSETDDY*A-C(Sx)-IIDEE-Z (SEQ ID NO: 5082), X-S[Nle]SETDDY*A-C(Sx)-IIDEE-Z (SEQ ID NO: 5083), X-SASETDDY*A-C(Sx)-IIDEE-Z (SEQ ID NO: 5084), X-SDSETDDY*A-C(Sx)-IIDEE-Z (SEQ ID NO: 5085), X-SEELDENY*V-C(Sx)-MNPNS-Z (SEQ ID NO: 954), X-SEGLSM-C(Sx)-NY*IGLINRIKK-Z (SEQ ID NO: 5086), X-SESETDDY*A-C(Sx)-IIDEE-Z (SEQ ID NO: 5087), X-SFSETDDY*A-C(Sx)-IIDEE-Z (SEQ ID NO: 5088), X-SGSETDDY*A-C(Sx)-IIDEE-Z (SEQ ID NO: 5089), X-SHDSEENY*V-C(Sx)-MNPNL-Z (SEQ ID NO: 956), X-SHSETDDY*A-C(Sx)-IIDEE-Z (SEQ ID NO: 5090), X-SISETDDY*A-C(Sx)-IIDEE-Z (SEQ ID NO: 5091), X-SKSETDDY*A-C(Sx)-IIDEE-Z (SEQ ID NO: 5092), X-SLSETDDY*A-C(Sx)-IIDEE-Z (SEQ ID NO: 5093), X-SNSETDDY*A-C(Sx)-IIDEE-Z (SEQ ID NO: 5094), X-SPAFD-C(Sx)-LY*[pY]WDQDPP-Z (SEQ ID NO: 989), X-SPAFD-C(Sx)-LY*YWDQDPP-Z (SEQ ID NO: 988), and X-SPATDLY*Q-C(Sx)-PPG-Z (SEQ ID NO: 5095); wherein X is H or acetyl; and Z is OH or NH2.

In some embodiments, the metal-binding compound of the present invention is selected from the group consisting of: X-SPQPEY*V-C(Sx)-QPDVR-Z (SEQ ID NO: 987), X-SPSETDDY*A-C(Sx)-IIDEE-Z (SEQ ID NO: 5096), X-SQSETDDY*A-C(Sx)-IIDEE-Z (SEQ ID NO: 5097), X-SRSETDDY*A-C(Sx)-IIDEE-Z (SEQ ID NO: 5098), X-SSEPVGIY*Q-C(Sx)-FEKKT-Z (SEQ ID NO: 962), X-SSHKG-C(Sx)-HY*KH-Z (SEQ ID NO: 935), X-SSHKGFHY*K-C(Sx)-G-Z (SEQ ID NO: 934), X-SSTDR-C(Sx)-PY*EKVSAGN-Z (SEQ ID NO: 960), X-STAENAEY*L-C(Sx)-VAPQS-Z (SEQ ID NO: 946), X-STAEN-C(Sx)-EY*LRVAPQS-Z (SEQ ID NO: 947), X-SV[Nle]ETDDY*A-C(Sx)-IIDEE-Z (SEQ ID NO: 5099), X-SVAETDDY*A-C(Sx)-IIDEE-Z (SEQ ID NO: 5100), X-SVDETDDY*A-C(Sx)-IIDEE-Z (SEQ ID NO: 5101), X-SVEETDDY*A-C(Sx)-IIDEE-Z (SEQ ID NO: 5102), X-SVFETDDY*A-C(Sx)-IIDEE-Z (SEQ ID NO: 5103), X-SVGETDDY*A-C(Sx)-IIDEE-Z (SEQ ID NO: 5104), X-SVHETDDY*A-C(Sx)-IIDEE-Z (SEQ ID NO: 5105), X-SVIETDDY*A-C(Sx)-IIDEE-Z (SEQ ID NO: 5106), X-SVKETDDY*A-C(Sx)-IIDEE-Z (SEQ ID NO: 5107), X-SVLETDDY*A-C(Sx)-IIDEE-Z (SEQ ID NO: 5108), X-SVNETDDY*A-C(Sx)-IIDEE-Z (SEQ ID NO: 5109), X-SVPETDDY*A-C(Sx)-IIDEE-Z (SEQ ID NO: 5110), X-SVQETDDY*A-C(Sx)-IIDEE-Z (SEQ ID NO: 5111), X-SVRETDDY*A-C(Sx)-IIDEE-Z (SEQ ID NO: 5112), X-SVS[Nle]TDDY*A-C(Sx)-IIDEE-Z (SEQ ID NO: 5113), X-SVSATDDY*A-C(Sx)-IIDEE-Z (SEQ ID NO: 5114), X-SVSDTDDY*A-C(Sx)-IIDEE-Z (SEQ ID NO: 5115), X-SVSE[Nle]DDY*A-C(Sx)-IIDEE-Z (SEQ ID NO: 5116), X-SVSEADDY*A-C(Sx)-IIDEE-Z (SEQ ID NO: 5117), X-SVSEDDDY*A-C(Sx)-IIDEE-Z (SEQ ID NO: 5118), X-SVSEEDDY*A-C(Sx)-IIDEE-Z (SEQ ID NO: 5119), X-SVSEFDDY*A-C(Sx)-IIDEE-Z (SEQ ID NO: 5120), X-SVSEGDDY*A-C(Sx)-IIDEE-Z (SEQ ID NO: 5121), X-SVSEHDDY*A-C(Sx)-IIDEE-Z (SEQ ID NO: 5122), X-SVSEIDDY*A-C(Sx)-IIDEE-Z (SEQ ID NO: 5123), X-SVSEKDDY*A-C(Sx)-IIDEE-Z (SEQ ID NO: 5124), X-SVSELDDY*A-C(Sx)-IIDEE-Z (SEQ ID NO: 5125), X-SVSENDDY*A-C(Sx)-IIDEE-Z (SEQ ID NO: 5126), X-SVSEPDDY*A-C(Sx)-IIDEE-Z (SEQ ID NO: 5127), X-SVSEQDDY*A-C(Sx)-IIDEE-Z (SEQ ID NO: 5128), and X-SVSERDDY*A-C(Sx)-IIDEE-Z (SEQ ID NO: 5129); wherein X— is H or acetyl; and Z is OH or NH2.

In some embodiments, the metal-binding compound of the present invention is selected from the group consisting of: X-SVSET[Nle]DY*A-C(Sx)-IIDEE-Z (SEQ ID NO: 5130), X-SVSETADY*A-C(Sx)-IIDEE-Z (SEQ ID NO: 5131), X-SVSETD[Nle]Y*A-C(Sx)-IIDEE-Z (SEQ ID NO: 5132), X-SVSETDAY*A-C(Sx)-IIDEE-Z (SEQ ID NO: 5133), X-SVSETDDY*[Nle]-C(Sx)-IIDEE-Z (SEQ ID NO: 5134), X-SVSETDDY*A-C(Sx)-[Nle]IDEE-Z (SEQ ID NO: 5135), X-SVSETDDY*A-C(Sx)-AIDEE-Z (SEQ ID NO: 5136), X-SVSETDDY*A-C(Sx)-DIDEE-Z (SEQ ID NO: 5137), X-SVSETDDY*A-C(Sx)-EIDEE-Z (SEQ ID NO: 5138), X-SVSETDDY*A-C(Sx)-FIDEE-Z (SEQ ID NO: 5139), X-SVSETDDY*A-C(Sx)-GIDEE-Z (SEQ ID NO: 5140), X-SVSETDDY*A-C(Sx)-HIDEE-Z (SEQ ID NO: 5141), X-SVSETDDY*A-C(Sx)-I[Nle]DEE-Z (SEQ ID NO: 5142), X-SVSETDDY*A-C(Sx)-IADEE-Z (SEQ ID NO: 5143), X-SVSETDDY*A-C(Sx)-IDDEE-Z (SEQ ID NO: 5144), X-SVSETDDY*A-C(Sx)-IEDEE-Z (SEQ ID NO: 5145), X-SVSETDDY*A-C(Sx)-IFDEE-Z (SEQ ID NO: 5146), X-SVSETDDY*A-C(Sx)-IGDEE-Z (SEQ ID NO: 5147), X-SVSETDDY*A-C(Sx)-IHDEE-Z (SEQ ID NO: 5148), X-SVSETDDY*A-C(Sx)-II[Nle]EE-Z (SEQ ID NO: 5149), X-SVSETDDY*A-C(Sx)-IIAEE-Z (SEQ ID NO: 5150), X-SVSETDDY*A-C(Sx)-IID[Nle]E-Z (SEQ ID NO: 5151), X-SVSETDDY*A-C(Sx)-IIDAE-Z (SEQ ID NO: 5152), X-SVSETDDY*A-C(Sx)-IIDDE-Z (SEQ ID NO: 5153), X-SVSETDDY*A-C(Sx)-IIDE[Nle]-Z (SEQ ID NO: 5154), X-SVSETDDY*A-C(Sx)-IIDEA-Z (SEQ ID NO: 5155), X-SVSETDDY*A-C(Sx)-IIDED-Z (SEQ ID NO: 5156), X-SVSETDDY*A-C(Sx)-IIDEF-Z (SEQ ID NO: 5157), X-SVSETDDY*A-C(Sx)-IIDEG-Z (SEQ ID NO: 5158), X-SVSETDDY*A-C(Sx)-IIDEH-Z (SEQ ID NO: 5159), X-SVSETDDY*A-C(Sx)-IIDEI-Z (SEQ ID NO: 5160), X-SVSETDDY*A-C(Sx)-IIDEK-Z (SEQ ID NO: 5161), X-SVSETDDY*A-C(Sx)-IIDEL-Z (SEQ ID NO: 5162), X-SVSETDDY*A-C(Sx)-IIDEN-Z (SEQ ID NO: 5163), X-SVSETDDY*A-C(Sx)-IIDEP-Z (SEQ ID NO: 5164), X-SVSETDDY*A-C(Sx)-IIDEQ-Z (SEQ ID NO: 5165), X-SVSETDDY*A-C(Sx)-IIDER-Z (SEQ ID NO: 5166), X-SVSETDDY*A-C(Sx)-IIDEV-Z (SEQ ID NO: 5167), X-SVSETDDY*A-C(Sx)-IIDEW-Z (SEQ ID NO: 5168), X-SVSETDDY*A-C(Sx)-IIDFE-Z (SEQ ID NO: 5169), X-SVSETDDY*A-C(Sx)-IIDGE-Z (SEQ ID NO: 5170), and X-SVSETDDY*A-C(Sx)-IIDHE-Z (SEQ ID NO: 5171); wherein X is H or acetyl; and Z is OH or NH2.

In some embodiments, the metal-binding compound of the present invention is selected from the group consisting of: X-SVSETDDY*A-C(Sx)-IIDIE-Z (SEQ ID NO: 5172), X-SVSETDDY*A-C(Sx)-IIDKE-Z (SEQ ID NO: 5173), X-SVSETDDY*A-C(Sx)-IIDLE-Z (SEQ ID NO: 5174), X-SVSETDDY*A-C(Sx)-IIDNE-Z (SEQ ID NO: 5175), X-SVSETDDY*A-C(Sx)-IIDPE-Z (SEQ ID NO: 5176), X-SVSETDDY*A-C(Sx)-IIDQE-Z (SEQ ID NO: 5177), X-SVSETDDY*A-C(Sx)-IIDRE-Z (SEQ ID NO: 5178), X-SVSETDDY*A-C(Sx)-IIDVE-Z (SEQ ID NO: 5179), X-SVSETDDY*A-C(Sx)-IIDWE-Z (SEQ ID NO: 5180), X-SVSETDDY*A-C(Sx)-IIEEE-Z (SEQ ID NO: 5181), X-SVSETDDY*A-C(Sx)-IIFEE-Z (SEQ ID NO: 5182), X-SVSETDDY*A-C(Sx)-IIGEE-Z (SEQ ID NO: 5183), X-SVSETDDY*A-C(Sx)-IIHEE-Z (SEQ ID NO: 5184), X-SVSETDDY*A-C(Sx)-IIIEE-Z (SEQ ID NO: 5185), X-SVSETDDY*A-C(Sx)-IIKEE-Z (SEQ ID NO: 5186), X-SVSETDDY*A-C(Sx)-IILEE-Z (SEQ ID NO: 5187), X-SVSETDDY*A-C(Sx)-IINEE-Z (SEQ ID NO: 5188), X-SVSETDDY*A-C(Sx)-IIPEE-Z (SEQ ID NO: 5189), X-SVSETDDY*A-C(Sx)-IIQEE-Z (SEQ ID NO: 5190), X-SVSETDDY*A-C(Sx)-IIREE-Z (SEQ ID NO: 5191), X-SVSETDDY*A-C(Sx)-IIVEE-Z (SEQ ID NO: 5192), X-SVSETDDY*A-C(Sx)-IIWEE-Z (SEQ ID NO: 5193), X-SVSETDDY*A-C(Sx)-IKDEE-Z (SEQ ID NO: 5194), X-SVSETDDY*A-C(Sx)-ILDEE-Z (SEQ ID NO: 5195), X-SVSETDDY*A-C(Sx)-INDEE-Z (SEQ ID NO: 5196), X-SVSETDDY*A-C(Sx)-IPDEE-Z (SEQ ID NO: 5197), X-SVSETDDY*A-C(Sx)-IQDEE-Z (SEQ ID NO: 5198), X-SVSETDDY*A-C(Sx)-IRDEE-Z (SEQ ID NO: 5199), X-SVSETDDY*A-C(Sx)-IVDEE-Z (SEQ ID NO: 5200), X-SVSETDDY*A-C(Sx)-IWDEE-Z (SEQ ID NO: 5201), X-SVSETDDY*A-C(Sx)-KIDEE-Z (SEQ ID NO: 5202), X-SVSETDDY*A-C(Sx)-LIDEE-Z (SEQ ID NO: 5203), X-SVSETDDY*A-C(Sx)-NIDEE-Z (SEQ ID NO: 5204), X-SVSETDDY*A-C(Sx)-PIDEE-Z (SEQ ID NO: 5205), X-SVSETDDY*A-C(Sx)-QIDEE-Z (SEQ ID NO: 5206), X-SVSETDDY*A-C(Sx)-RIDEE-Z (SEQ ID NO: 5207), X-SVSETDDY*A-C(Sx)-VIDEE-Z (SEQ ID NO: 5208), X-SVSETDDY*A-C(Sx)-WIDEE-Z (SEQ ID NO: 5209), X-SVSETDDY*D-C(Sx)-IIDEE-Z (SEQ ID NO: 5210), X-SVSETDDY*E-C(Sx)-IIDEE-Z (SEQ ID NO: 5211), X-SVSETDDY*F-C(Sx)-IIDEE-Z (SEQ ID NO: 5212), X-SVSETDDY*G-C(Sx)-IIDEE-Z (SEQ ID NO: 5213), X-SVSETDDY*H-C(Sx)-IIDEE-Z (SEQ ID NO: 5214), X-SVSETDDY*I-C(Sx)-IIDEE-Z (SEQ ID NO: 5215), X-SVSETDDY*K-C(Sx)-IIDEE-Z (SEQ ID NO: 5216), X-SVSETDDY*L-C(Sx)-IIDEE-Z (SEQ ID NO: 5217), X-SVSETDDY*N-C(Sx)-IIDEE-Z (SEQ ID NO: 5218), X-SVSETDDY*P-C(Sx)-IIDEE-Z (SEQ ID NO: 5219), X-SVSETDDY*Q-C(Sx)-IIDEE-Z (SEQ ID NO: 5220), X-SVSETDDY*R-C(Sx)-IIDEE-Z (SEQ ID NO: 5221), X-SVSETDDY*V-C(Sx)-IIDEE-Z (SEQ ID NO: 5222), X-SVSETDDY*W-C(Sx)-IIDEE-Z (SEQ ID NO: 5223), X-SVSETDEY*A-C(Sx)-IIDEE-Z (SEQ ID NO: 5224), X-SVSETDFY*A-C(Sx)-IIDEE-Z (SEQ ID NO: 5225), X-SVSETDGY*A-C(Sx)-IIDEE-Z (SEQ ID NO: 5226), X-SVSETDHY*A-C(Sx)-IIDEE-Z (SEQ ID NO: 5227), X-SVSETDIY*A-C(Sx)-IIDEE-Z (SEQ ID NO: 5228), X-SVSETDKY*A-C(Sx)-IIDEE-Z (SEQ ID NO: 5229), X-SVSETDLY*A-C(Sx)-IIDEE-Z (SEQ ID NO: 5230), X-SVSETDNY*A-C(Sx)-IIDEE-Z (SEQ ID NO: 5231), X-SVSETDPY*A-C(Sx)-IIDEE-Z (SEQ ID NO: 5232), X-SVSETDQY*A-C(Sx)-IIDEE-Z (SEQ ID NO: 5233), X-SVSETDRY*A-C(Sx)-IIDEE-Z (SEQ ID NO: 5234), X-SVSETDVY*A-C(Sx)-IIDEE-Z (SEQ ID NO: 5235), and X-SVSETDWY*A-C(Sx)-IIDEE-Z (SEQ ID NO: 5236); wherein X is H or acetyl; and Z is OH or NH2.

In some embodiments, the metal-binding compound of the present invention is selected from the group consisting of: X-SVSETEDY*A-C(Sx)-IIDEE-Z (SEQ ID NO: 5237), X-SVSETFDY*A-C(Sx)-IIDEE-Z (SEQ ID NO: 5238), X-SVSETGDY*A-C(Sx)-IIDEE-Z (SEQ ID NO: 5239), X-SVSETHDY*A-C(Sx)-IIDEE-Z (SEQ ID NO: 5240), X-SVSETIDY*A-C(Sx)-IIDEE-Z (SEQ ID NO: 5241), X-SVSETKDY*A-C(Sx)-IIDEE-Z (SEQ ID NO: 5242), X-SVSETLDY*A-C(Sx)-IIDEE-Z (SEQ ID NO: 5243), X-SVSETNDY*A-C(Sx)-IIDEE-Z (SEQ ID NO: 5244), X-SVSETPDY*A-C(Sx)-IIDEE-Z (SEQ ID NO: 5245), X-SVSETQDY*A-C(Sx)-IIDEE-Z (SEQ ID NO: 5246), X-SVSETRDY*A-C(Sx)-IIDEE-Z (SEQ ID NO: 5247), X-SVSETVDY*A-C(Sx)-IIDEE-Z (SEQ ID NO: 5248), X-SVSETWDY*A-C(Sx)-IIDEE-Z (SEQ ID NO: 5249), X-SVSEVDDY*A-C(Sx)-IIDEE-Z (SEQ ID NO: 5250), X-SVSEWDDY*A-C(Sx)-IIDEE-Z (SEQ ID NO: 5251), X-SVSFTDDY*A-C(Sx)-IIDEE-Z (SEQ ID NO: 5252), X-SVSGTDDY*A-C(Sx)-IIDEE-Z (SEQ ID NO: 5253), X-SVSHTDDY*A-C(Sx)-IIDEE-Z (SEQ ID NO: 5254), X-SVSITDDY*A-C(Sx)-IIDEE-Z (SEQ ID NO: 5255), X-SVSKTDDY*A-C(Sx)-IIDEE-Z (SEQ ID NO: 5256), X-SVSLTDDY*A-C(Sx)-IIDEE-Z (SEQ ID NO: 5257), X-SVSNTDDY*A-C(Sx)-IIDEE-Z (SEQ ID NO: 5258), X-SVSPTDDY*A-C(Sx)-IIDEE-Z (SEQ ID NO: 5259), X-SVSQTDDY*A-C(Sx)-IIDEE-Z (SEQ ID NO: 5260), X-SVSRTDDY*A-C(Sx)-IIDEE-Z (SEQ ID NO: 5261), X-SVSVTDDY*A-C(Sx)-IIDEE-Z (SEQ ID NO: 5262), X-SVSWTDDY*A-C(Sx)-IIDEE-Z (SEQ ID NO: 5263), X-SVVETDDY*A-C(Sx)-IIDEE-Z (SEQ ID NO: 5264), X-SVWETDDY*A-C(Sx)-IIDEE-Z (SEQ ID NO: 5265), X-SWSETDDY*A-C(Sx)-IIDEE-Z (SEQ ID NO: 5266), X-TDGEDADY*T-C(Sx)-FTNQQ-Z (SEQ ID NO: 933), X-TDVE-C(Sx)-TY*ADFIASG-Z (SEQ ID NO: 964), X-TEADGELY*V-C(Sx)-NTPSG-Z (SEQ ID NO: 952), X-TEERLPSS*P-C(Sx)-YEDAA-Z (SEQ ID NO: 1034), X-TENDDDVY*R-C(Sx)-LEELA-Z (SEQ ID NO: 984), X-TFLPV-C(Sx)-EY*INQSVPK-Z (SEQ ID NO: 943), X-TFLPVPEY*I-C(Sx)-QSVPK-Z (SEQ ID NO: 942), X-TG[Nle]FPRNY*V-C(Sx)-PVNRN-Z (SEQ ID NO: 5267), X-TLMEKDSY*P-C(Sx)-FLKSP-Z (SEQ ID NO: 972), X-TPENN-C(Sx)-EY*AKVSGV-Z (SEQ ID NO: 5268), X-TQEQYELY*S-C(Sx)-MGSTF-Z (SEQ ID NO: 931), X-TVDGKEIY*N-C(Sx)-IRRKT-Z (SEQ ID NO: 971), X-VADERVDY*V-C(Sx)-VDQQK-Z (SEQ ID NO: 958), X-VEEEEY*I-C(Sx)-Z (SEQ ID NO: 5269), X-VEEEY*I-C(Sx)-IV-Z (SEQ ID NO: 5270), X-VQPSPARSSS*Y-C(Sx)-EANE-Z (SEQ ID NO: 5271), X-VSETDDY*A-C(Sx)-IIDEEDT-Z (SEQ ID NO: 5272), X-VTRRT-C(Sx)-DY*FL-Z (SEQ ID NO: 968), X-VVSETDDY*A-C(Sx)-IIDEE-Z (SEQ ID NO: 5273), X-VY*S-C(Sx)-DY[pY]RLFNPSKKK-Z (SEQ ID NO: 5274), X-VY*S-C(Sx)-DYYRLFNPSKKK-Z (SEQ ID NO: 5275), X-VYS-C(Sx)-DY*[pY]RLFNPSKKK-Z (SEQ ID NO: 913), X-VYS-C(Sx)-DY*YRLFNPSKKK-Z (SEQ ID NO: 912), X-WEEEEY*I-C(Sx)-Z (SEQ ID NO: 5276), X-WEEEY*I-C(Sx)-IV-Z (SEQ ID NO: 5277), and X-WVSETDDY*A-C(Sx)-IIDEE-Z (SEQ ID NO: 5278); wherein X— is H or acetyl; and Z is OH or NH2.

In some embodiments, the metal-binding compound of the present invention is selected from the group consisting of: X-EPDY*I-C(Sx)-FG-Z (SEQ ID NO: 5279); X-PDY*I-C(Sx)-FG-Z (SEQ ID NO: 5280); X-KKHTDDGY*M-C(Sx)-MSPGVA-Z (SEQ ID NO: 5281); X-KKHTD-C(Sx)-GY*MPMSPGVA-Z (SEQ ID NO: 5282); X-KKHT-C(Sx)-DGY*MPMSPGVA-Z (SEQ ID NO: 5283); X-KKHTDDGY*[Nle]-C(Sx)-[Nle]SPGVA-Z (SEQ ID NO: 5284); X-KKHTD-C(Sx)-GY*[Nle]P[Nle]SPGVA-Z (SEQ ID NO: 5285); X-KKHT-C(Sx)-DGY*[Nle]P[Nle]SPGVA-Z (SEQ ID NO: 5286); X-KKAEEEEY*I-C(Sx)-LV-Z (SEQ ID NO: 5287); X-RRRAEEEEY*I-C(Sx)-LV-Z (SEQ ID NO: 5288); X-RRAEEEEY*I-C(Sx)-LV-Z (SEQ ID NO: 5289); X-[Nle]-C(Sx)-DY*[Nle]TMQIGKK-Z (SEQ ID NO: 5290); X-A-C(Sx)-DY*[Nle]TMQIGKK-Z (SEQ ID NO: 5291); X-D-C(Sx)-DY*[Nle]TMQIGKK-Z (SEQ ID NO: 5292); X-E-C(Sx)-DY*[Nle]TMQIGKK-Z (SEQ ID NO: 5293); X-F-C(Sx)-DY*[Nle]TMQIGKK-Z (SEQ ID NO: 5294); X-G-C(Sx)-DY*[Nle]TMQIGKK-Z (SEQ ID NO: 5295); X-H-C(Sx)-DY*[Nle]TMQIGKK-Z (SEQ ID NO: 5296); X-I-C(Sx)-DY*[Nle]TMQIGKK-Z (SEQ ID NO: 5297); X-K-C(Sx)-DY*[Nle]TMQIGKK-Z (SEQ ID NO: 5298); X-L-C(Sx)-DY*[Nle]TMQIGKK-Z (SEQ ID NO: 5299); X-N-C(Sx)-DY*[Nle]TMQIGKK-Z (SEQ ID NO: 5300); X-P-C(Sx)-DY*[Nle]TMQIGKK-Z (SEQ ID NO: 5301); X-Q-C(Sx)-DY*[Nle]TMQIGKK-Z (SEQ ID NO: 5302); X-R-C(Sx)-[Nle]Y*[Nle]TMQIGKK-Z (SEQ ID NO: 5303); X-R-C(Sx)-AY*[Nle]TMQIGKK-Z (SEQ ID NO: 5304); X-R-C(Sx)-DY*[Nle][Nle]MQIGKK-Z (SEQ ID NO: 5305); X-R-C(Sx)-DY*[Nle]AMQIGKK-Z (SEQ ID NO: 5306); X-R-C(Sx)-DY*[Nle]DMQIGKK-Z (SEQ ID NO: 5307); X-R-C(Sx)-DY*[Nle]EMQIGKK-Z (SEQ ID NO: 5308); X-R-C(Sx)-DY*[Nle]FMQIGKK-Z (SEQ ID NO: 5309); X-R-C(Sx)-DY*[Nle]GMQIGKK-Z (SEQ ID NO: 5310); X-R-C(Sx)-DY*[Nle]HMQIGKK-Z (SEQ ID NO: 5311); X-R-C(Sx)-DY*[Nle]IMQIGKK-Z (SEQ ID NO: 5312); X-R-C(Sx)-DY*[Nle]KMQIGKK-Z (SEQ ID NO: 5313); X-R-C(Sx)-DY*[Nle]LMQIGKK-Z (SEQ ID NO: 5314); X-R-C(Sx)-DY*[Nle]NMQIGKK-Z (SEQ ID NO: 5315); X-R-C(Sx)-DY*[Nle]PMQIGKK-Z (SEQ ID NO: 5316); X-R-C(Sx)-DY*[Nle]QMQIGKK-Z (SEQ ID NO: 5317); X-R-C(Sx)-DY*[Nle]RMQIGKK-Z (SEQ ID NO: 5318); wherein X— is H or acetyl; and Z is OH or NH2.

In some embodiments, the metal-binding compound of the present invention is selected from the group consisting of: X-R-C(Sx)-DY*[Nle]T[Nle]QIGKK-Z (SEQ ID NO: 5319); X-R-C(Sx)-DY*[Nle]TAQIGKK-Z (SEQ ID NO: 5320); X-R-C(Sx)-DY*[Nle]TDQIGKK-Z (SEQ ID NO: 5321); X-R-C(Sx)-DY*[Nle]TEQIGKK-Z (SEQ ID NO: 5322); X-R-C(Sx)-DY*[Nle]TFQIGKK-Z (SEQ ID NO: 5323); X-R-C(Sx)-DY*[Nle]TGQIGKK-Z (SEQ ID NO: 5324); X-R-C(Sx)-DY*[Nle]THQIGKK-Z (SEQ ID NO: 5325); X-R-C(Sx)-DY*[Nle]TIQIGKK-Z (SEQ ID NO: 5326); X-R-C(Sx)-DY*[Nle]TKQIGKK-Z (SEQ ID NO: 5327); X-R-C(Sx)-DY*[Nle]TLQIGKK-Z (SEQ ID NO: 5328); X-R-C(Sx)-DY*[Nle]TM[Nle]IGKK-Z (SEQ ID NO: 5329); X-R-C(Sx)-DY*[Nle]TMAIGKK-Z (SEQ ID NO: 5330); X-R-C(Sx)-DY*[Nle]TMDIGKK-Z (SEQ ID NO: 5331); X-R-C(Sx)-DY*[Nle]TMEIGKK-Z (SEQ ID NO: 5332); X-R-C(Sx)-DY*[Nle]TMFIGKK-Z (SEQ ID NO: 5333); X-R-C(Sx)-DY*[Nle]TMGIGKK-Z (SEQ ID NO: 5334); X-R-C(Sx)-DY*[Nle]TMHIGKK-Z (SEQ ID NO: 5335); X-R-C(Sx)-DY*[Nle]TMIIGKK-Z (SEQ ID NO: 5336); X-R-C(Sx)-DY*[Nle]TMKIGKK-Z (SEQ ID NO: 5337); X-R-C(Sx)-DY*[Nle]TMLIGKK-Z (SEQ ID NO: 5338); X-R-C(Sx)-DY*[Nle]TMNIGKK-Z (SEQ ID NO: 5339); X-R-C(Sx)-DY*[Nle]TMPIGKK-Z (SEQ ID NO: 5340); X-R-C(Sx)-DY*[Nle]TMQ[Nle]GKK-Z (SEQ ID NO: 5341); X-R-C(Sx)-DY*[Nle]TMQAGKK-Z (SEQ ID NO: 5342); X-R-C(Sx)-DY*[Nle]TMQDGKK-Z (SEQ ID NO: 5343); X-R-C(Sx)-DY*[Nle]TMQEGKK-Z (SEQ ID NO: 5344); X-R-C(Sx)-DY*[Nle]TMQFGKK-Z (SEQ ID NO: 5345); X-R-C(Sx)-DY*[Nle]TMQGGKK-Z (SEQ ID NO: 5346); X-R-C(Sx)-DY*[Nle]TMQHGKK-Z (SEQ ID NO: 5347); X-R-C(Sx)-DY*[Nle]TMQI[Nle]KK-Z (SEQ ID NO: 5348); X-R-C(Sx)-DY*[Nle]TMQIAKK-Z (SEQ ID NO: 5349); X-R-C(Sx)-DY*[Nle]TMQIDKK-Z (SEQ ID NO: 5350); X-R-C(Sx)-DY*[Nle]TMQIEKK-Z (SEQ ID NO: 5351); X-R-C(Sx)-DY*[Nle]TMQIFKK-Z (SEQ ID NO: 5352); X-R-C(Sx)-DY*[Nle]TMQIG[Nle]K-Z (SEQ ID NO: 5353); X-R-C(Sx)-DY*[Nle]TMQIGAK-Z (SEQ ID NO: 5354); wherein X— is H or acetyl; and Z is OH or NH2.

In some embodiments, the metal-binding compound of the present invention is selected from the group consisting of: X-R-C(Sx)-DY*[Nle]TMQIGDK-Z (SEQ ID NO: 5355); X-R-C(Sx)-DY*[Nle]TMQIGEK-Z (SEQ ID NO: 5356); X-R-C(Sx)-DY*[Nle]TMQIGFK-Z (SEQ ID NO: 5357); X-R-C(Sx)-DY*[Nle]TMQIGGK-Z (SEQ ID NO: 5358); X-R-C(Sx)-DY*[Nle]TMQIGHK-Z (SEQ ID NO: 5359); X-R-C(Sx)-DY*[Nle]TMQIGIK-Z (SEQ ID NO: 5360); X-R-C(Sx)-DY*[Nle]TMQIGK[Nle]-Z (SEQ ID NO: 5361); X-R-C(Sx)-DY*[Nle]TMQIGKA-Z (SEQ ID NO: 5362); X-R-C(Sx)-DY*[Nle]TMQIGKD-Z (SEQ ID NO: 5363); X-R-C(Sx)-DY*[Nle]TMQIGKE-Z (SEQ ID NO: 5364); X-R-C(Sx)-DY*[Nle]TMQIGKF-Z (SEQ ID NO: 5365); X-R-C(Sx)-DY*[Nle]TMQIGKG-Z (SEQ ID NO: 5366); X-R-C(Sx)-DY*[Nle]TMQIGKH-Z (SEQ ID NO: 5367); X-R-C(Sx)-DY*[Nle]TMQIGKI-Z (SEQ ID NO: 5368); X-R-C(Sx)-DY*[Nle]TMQIGKL-Z (SEQ ID NO: 5369); X-R-C(Sx)-DY*[Nle]TMQIGKN-Z (SEQ ID NO: 5370); X-R-C(Sx)-DY*[Nle]TMQIGKP-Z (SEQ ID NO: 5371); X-R-C(Sx)-DY*[Nle]TMQIGKQ-Z (SEQ ID NO: 5372); X-R-C(Sx)-DY*[Nle]TMQIGKR-Z (SEQ ID NO: 5373); X-R-C(Sx)-DY*[Nle]TMQIGKV-Z (SEQ ID NO: 5374); X-R-C(Sx)-DY*[Nle]TMQIGKW-Z (SEQ ID NO: 5375); X-R-C(Sx)-DY*[Nle]TMQIGLK-Z (SEQ ID NO: 5376); X-R-C(Sx)-DY*[Nle]TMQIGNK-Z (SEQ ID NO: 5377); X-R-C(Sx)-DY*[Nle]TMQIGPK-Z (SEQ ID NO: 5378); X-R-C(Sx)-DY*[Nle]TMQIGQK-Z (SEQ ID NO: 5379); X-R-C(Sx)-DY*[Nle]TMQIGRK-Z (SEQ ID NO: 5380); X-R-C(Sx)-DY*[Nle]TMQIGVK-Z (SEQ ID NO: 5381); X-R-C(Sx)-DY*[Nle]TMQIGWK-Z (SEQ ID NO: 5382); X-R-C(Sx)-DY*[Nle]TMQIHKK-Z (SEQ ID NO: 5383); X-R-C(Sx)-DY*[Nle]TMQIIKK-Z (SEQ ID NO: 5384); X-R-C(Sx)-DY*[Nle]TMQIKKK-Z (SEQ ID NO: 5385); X-R-C(Sx)-DY*[Nle]TMQILKK-Z (SEQ ID NO: 5386); X-R-C(Sx)-DY*[Nle]TMQINKK-Z (SEQ ID NO: 5387); X-R-C(Sx)-DY*[Nle]TMQIPKK-Z (SEQ ID NO: 5388); X-R-C(Sx)-DY*[Nle]TMQIQKK-Z (SEQ ID NO: 5389); X-R-C(Sx)-DY*[Nle]TMQIRKK-Z (SEQ ID NO: 5390); X-R-C(Sx)-DY*[Nle]TMQIVKK-Z (SEQ ID NO: 5391); wherein X— is H or acetyl; and Z is OH or NH2.

In some embodiments, the metal-binding compound of the present invention is selected from the group consisting of: X-R-C(Sx)-DY*[Nle]TMQIWKK-Z (SEQ ID NO: 5392); X-R-C(Sx)-DY*[Nle]TMQKGKK-Z (SEQ ID NO: 5393); X-R-C(Sx)-DY*[Nle]TMQLGKK-Z (SEQ ID NO: 5394); X-R-C(Sx)-DY*[Nle]TMQNGKK-Z (SEQ ID NO: 5395); X-R-C(Sx)-DY*[Nle]TMQPGKK-Z (SEQ ID NO: 5396); X-R-C(Sx)-DY*[Nle]TMQQGKK-Z (SEQ ID NO: 5397); X-R-C(Sx)-DY*[Nle]TMQRGKK-Z (SEQ ID NO: 5398); X-R-C(Sx)-DY*[Nle]TMQVGKK-Z (SEQ ID NO: 5399); X-R-C(Sx)-DY*[Nle]TMQWGKK-Z (SEQ ID NO: 5400); X-R-C(Sx)-DY*[Nle]TMRIGKK-Z (SEQ ID NO: 5401); X-R-C(Sx)-DY*[Nle]TMVIGKK-Z (SEQ ID NO: 5402); X-R-C(Sx)-DY*[Nle]TMWIGKK-Z (SEQ ID NO: 5403); X-R-C(Sx)-DY*[Nle]TNQIGKK-Z (SEQ ID NO: 5404); X-R-C(Sx)-DY*[Nle]TPQIGKK-Z (SEQ ID NO: 5405); X-R-C(Sx)-DY*[Nle]TQQIGKK-Z (SEQ ID NO: 5406); X-R-C(Sx)-DY*[Nle]TRQIGKK-Z (SEQ ID NO: 5407); X-R-C(Sx)-DY*[Nle]TVQIGKK-Z (SEQ ID NO: 5408); X-R-C(Sx)-DY*[Nle]TWQIGKK-Z (SEQ ID NO: 5409); X-R-C(Sx)-DY*[Nle]VMQIGKK-Z (SEQ ID NO: 5410); X-R-C(Sx)-DY*[Nle]WMQIGKK-Z (SEQ ID NO: 5411); X-R-C(Sx)-DY*ATMQIGKK-Z (SEQ ID NO: 5412); X-R-C(Sx)-DY*DTMQIGKK-Z (SEQ ID NO: 5413); X-R-C(Sx)-DY*ETMQIGKK-Z (SEQ ID NO: 5414); X-R-C(Sx)-DY*FTMQIGKK-Z (SEQ ID NO: 5415); X-R-C(Sx)-DY*GTMQIGKK-Z (SEQ ID NO: 5416); X-R-C(Sx)-DY*HTMQIGKK-Z (SEQ ID NO: 5417); X-R-C(Sx)-DY*ITMQIGKK-Z (SEQ ID NO: 5418); X-R-C(Sx)-DY*KTMQIGKK-Z (SEQ ID NO: 5419); X-R-C(Sx)-DY*LTMQIGKK-Z (SEQ ID NO: 5420); X-R-C(Sx)-DY*NTMQIGKK-Z (SEQ ID NO: 5421); X-R-C(Sx)-DY*PTMQIGKK-Z (SEQ ID NO: 5422); X-R-C(Sx)-DY*QTMQIGKK-Z (SEQ ID NO: 5423); X-R-C(Sx)-DY*RTMQIGKK-Z (SEQ ID NO: 5424); X-R-C(Sx)-DY*VTMQIGKK-Z (SEQ ID NO: 5425); X-R-C(Sx)-DY*WTMQIGKK-Z (SEQ ID NO: 5426); X-R-C(Sx)-EY*[Nle]TMQIGKK-Z (SEQ ID NO: 5427); X-R-C(Sx)-FY*[Nle]TMQIGKK-Z (SEQ ID NO: 5428); X-R-C(Sx)-GY*[Nle]TMQIGKK-Z (SEQ ID NO: 5429); wherein X— is H or acetyl; and Z is OH or NH2.

In some embodiments, the metal-binding compound of the present invention is selected from the group consisting of: X-R-C(Sx)-HY*[Nle]TMQIGKK-Z (SEQ ID NO: 5430); X-R-C(Sx)-IY*[Nle]TMQIGKK-Z (SEQ ID NO: 5431); X-R-C(Sx)-KY*[Nle]TMQIGKK-Z (SEQ ID NO: 5432); X-R-C(Sx)-LY*[Nle]TMQIGKK-Z (SEQ ID NO: 5433); X-R-C(Sx)-NY*[Nle]TMQIGKK-Z (SEQ ID NO: 5434); X-R-C(Sx)-PY*[Nle]TMQIGKK-Z (SEQ ID NO: 5435); X-R-C(Sx)-QY*[Nle]TMQIGKK-Z (SEQ ID NO: 5436); X-R-C(Sx)-RY*[Nle]TMQIGKK-Z (SEQ ID NO: 5437); X-R-C(Sx)-VY*[Nle]TMQIGKK-Z (SEQ ID NO: 5438); X-V-C(Sx)-DY*[Nle]TMQIGKK-Z (SEQ ID NO: 5439); X-KKHTDDGYM-C(Sx)-MS*PGVA-Z (SEQ ID NO: 5440); X-KKHT*D-C(Sx)-GYMPMSPGVA-Z (SEQ ID NO: 5441); X-KKHTDDGY[Nle]-C(Sx)-[Nle]S*PGVA-Z (SEQ ID NO: 5442); X-KKHT*D-C(Sx)-GY[Nle]P[Nle]SPGVA-Z (SEQ ID NO: 5443); X-KKKKVKKRPQRAHSD-C(Sx)-FS*-Z (SEQ ID NO: 3473); X-EDFSS*I-C(Sx)-DMDFSALLSQISS (SEQ ID NO: 1720); X-GDQDYLS*L-C(Sx)-VG-Z (SEQ ID NO: 651); X-KKRAARATS*-C(Sx)-VFA-Z (SEQ ID NO: 5444); X-ARKRRRHPS*G-C(Sx)-PTA-Z (SEQ ID NO: 1299); X-ARKRRRHPS*G-C(Sx)-PT-Z (SEQ ID NO: 5445); X-ARKRRRHPS*G-C(Sx)-P-Z (SEQ ID NO: 5446); X-ARKRRRHPS*G[dP]-C(Sx)-Z (SEQ ID NO: 5447); X-ARKRRRHPS*GP-C(Sx)-Z (SEQ ID NO: 5448); X-ARKRRRHPS*G-C(Sx)-Z (SEQ ID NO: 5449); X-RRRQFS*L-C(Sx)-Z (SEQ ID NO: 5450); X-KKERLLDDRHDS*G-C(Sx)-DDMKDEE-Z (SEQ ID NO: 5451); X-KKERLLDDRHDS*G-C(Sx)-DEMKDEE-Z (SEQ ID NO: 5452); X-KKERLLDDRHDS*G-C(Sx)-DAMKDEE-Z (SEQ ID NO: 5453); X-KKERLLDDRHDDG-C(Sx)-DS*MKDEE-Z (SEQ ID NO: 5454); X-KKERLLDDRHDEG-C(Sx)-DS*MKDEE-Z (SEQ ID NO: 5455); X-KKERLLDDRHDAG-C(Sx)-DS*MKDEE-Z (SEQ ID NO: 5456); wherein X— is H or acetyl; and Z is OH or NH2.

In some embodiments, the metal-binding compound of the present invention is selected from the group consisting of: X-[Nle]KR-C(Sx)-AS*FKKFA-Z (SEQ ID NO: 5457); X-AKR-C(Sx)-AS*FKKFA-Z (SEQ ID NO: 5458); X-DKR-C(Sx)-AS*FKKFA-Z (SEQ ID NO: 5459); X-EKR-C(Sx)-AS*FKKFA-Z (SEQ ID NO: 5460); X-FKR-C(Sx)-AS*FKKFA-Z (SEQ ID NO: 5461); X-GKR-C(Sx)-AS*FKKFA-Z (SEQ ID NO: 5462); X-HKR-C(Sx)-AS*FKKFA-Z (SEQ ID NO: 5463); X-IKR-C(Sx)-AS*FKKFA-Z (SEQ ID NO: 5464); X-KKR-C(Sx)-AS*FKKFA-Z (SEQ ID NO: 5465); X-L[Nle]R-C(Sx)-AS*FKKFA-Z (SEQ ID NO: 5466); X-LAR-C(Sx)-AS*FKKFA-Z (SEQ ID NO: 5467); X-LDR-C(Sx)-AS*FKKFA-Z (SEQ ID NO: 5468); X-LER-C(Sx)-AS*FKKFA-Z (SEQ ID NO: 5469); X-LFR-C(Sx)-AS*FKKFA-Z (SEQ ID NO: 5470); X-LGR-C(Sx)-AS*FKKFA-Z (SEQ ID NO: 5471); X-LHR-C(Sx)-AS*FKKFA-Z (SEQ ID NO: 5472); X-LIR-C(Sx)-AS*FKKFA-Z (SEQ ID NO: 5473); X-LK[Nle]-C(Sx)-AS*FKKFA-Z (SEQ ID NO: 5474); X-LKA-C(Sx)-AS*FKKFA-Z (SEQ ID NO: 5475); X-LKD-C(Sx)-AS*FKKFA-Z (SEQ ID NO: 5476); X-LKE-C(Sx)-AS*FKKFA-Z (SEQ ID NO: 5477); X-LKF-C(Sx)-AS*FKKFA-Z (SEQ ID NO: 5478); X-LKG-C(Sx)-AS*FKKFA-Z (SEQ ID NO: 5479); X-LKH-C(Sx)-AS*FKKFA-Z (SEQ ID NO: 5480); X-LKI-C(Sx)-AS*FKKFA-Z (SEQ ID NO: 5481); X-LKK-C(Sx)-AS*FKKFA-Z (SEQ ID NO: 5482); X-LKL-C(Sx)-AS*FKKFA-Z (SEQ ID NO: 5483); X-LKN-C(Sx)-AS*FKKFA-Z (SEQ ID NO: 5484); X-LKP-C(Sx)-AS*FKKFA-Z (SEQ ID NO: 5485); X-LKQ-C(Sx)-AS*FKKFA-Z (SEQ ID NO: 5486); wherein X— is H or acetyl; and Z is OH or NH2.

In some embodiments, the metal-binding compound of the present invention is selected from the group consisting of: X-LKR-C(Sx)-[Nle]S*FKKFA-Z (SEQ ID NO: 5487); X-LKR-C(Sx)-AS*[Nle]KKFA-Z (SEQ ID NO: 5488); X-LKR-C(Sx)-AS*AKKFA-Z (SEQ ID NO: 5489); X-LKR-C(Sx)-AS*DKKFA-Z (SEQ ID NO: 5490); X-LKR-C(Sx)-AS*EKKFA-Z (SEQ ID NO: 5491); X-LKR-C(Sx)-AS*F[Nle]KFA-Z (SEQ ID NO: 5492); X-LKR-C(Sx)-AS*FAKFA-Z (SEQ ID NO: 5493); X-LKR-C(Sx)-AS*FDKFA-Z (SEQ ID NO: 5494); X-LKR-C(Sx)-AS*FEKFA-Z (SEQ ID NO: 5495); X-LKR-C(Sx)-AS*FFKFA-Z (SEQ ID NO: 5496); X-LKR-C(Sx)-AS*FGKFA-Z (SEQ ID NO: 5497); X-LKR-C(Sx)-AS*FHKFA-Z (SEQ ID NO: 5498); X-LKR-C(Sx)-AS*FIKFA-Z (SEQ ID NO: 5499); X-LKR-C(Sx)-AS*FK[Nle]FA-Z (SEQ ID NO: 5500); X-LKR-C(Sx)-AS*FKAFA-Z (SEQ ID NO: 5501); X-LKR-C(Sx)-AS*FKDFA-Z (SEQ ID NO: 5502); X-LKR-C(Sx)-AS*FKEFA-Z (SEQ ID NO: 5503); X-LKR-C(Sx)-AS*FKFFA-Z (SEQ ID NO: 5504); X-LKR-C(Sx)-AS*FKGFA-Z (SEQ ID NO: 5505); X-LKR-C(Sx)-AS*FKHFA-Z (SEQ ID NO: 5506); X-LKR-C(Sx)-AS*FKIFA-Z (SEQ ID NO: 5507); X-LKR-C(Sx)-AS*FKK[Nle]A-Z (SEQ ID NO: 5508); X-LKR-C(Sx)-AS*FKKAA-Z (SEQ ID NO: 5509); X-LKR-C(Sx)-AS*FKKDA-Z (SEQ ID NO: 5510); X-LKR-C(Sx)-AS*FKKEA-Z (SEQ ID NO: 5511); X-LKR-C(Sx)-AS*FKKF[Nle]-Z (SEQ ID NO: 5512); X-LKR-C(Sx)-AS*FKKFD-Z (SEQ ID NO: 5513); X-LKR-C(Sx)-AS*FKKFE-Z (SEQ ID NO: 5514); X-LKR-C(Sx)-AS*FKKFF-Z (SEQ ID NO: 5515); X-LKR-C(Sx)-AS*FKKFG-Z (SEQ ID NO: 5516); X-LKR-C(Sx)-AS*FKKFH-Z (SEQ ID NO: 5517); X-LKR-C(Sx)-AS*FKKFI-Z (SEQ ID NO: 5518); X-LKR-C(Sx)-AS*FKKFK-Z (SEQ ID NO: 5519); X-LKR-C(Sx)-AS*FKKFL-Z (SEQ ID NO: 5520); X-LKR-C(Sx)-AS*FKKFN-Z (SEQ ID NO: 5521); X-LKR-C(Sx)-AS*FKKFP-Z (SEQ ID NO: 5522); X-LKR-C(Sx)-AS*FKKFQ-Z (SEQ ID NO: 5523); X-LKR-C(Sx)-AS*FKKFR-Z (SEQ ID NO: 5524); X-LKR-C(Sx)-AS*FKKFV-Z (SEQ ID NO: 5525); wherein X— is H or acetyl; and Z is OH or NH2.

In some embodiments, the metal-binding compound of the present invention is selected from the group consisting of: X-LKR-C(Sx)-AS*FKKFW-Z (SEQ ID NO: 5526); X-LKR-C(Sx)-AS*FKKGA-Z (SEQ ID NO: 5527); X-LKR-C(Sx)-AS*FKKHA-Z (SEQ ID NO: 5528); X-LKR-C(Sx)-AS*FKKIA-Z (SEQ ID NO: 5529); X-LKR-C(Sx)-AS*FKKKA-Z (SEQ ID NO: 5530); X-LKR-C(Sx)-AS*FKKLA-Z (SEQ ID NO: 5531); X-LKR-C(Sx)-AS*FKKNA-Z (SEQ ID NO: 5532); X-LKR-C(Sx)-AS*FKKPA-Z (SEQ ID NO: 5533); X-LKR-C(Sx)-AS*FKKQA-Z (SEQ ID NO: 5534); X-LKR-C(Sx)-AS*FKKRA-Z (SEQ ID NO: 5535); X-LKR-C(Sx)-AS*FKKVA-Z (SEQ ID NO: 5536); X-LKR-C(Sx)-AS*FKKWA-Z (SEQ ID NO: 5537); X-LKR-C(Sx)-AS*FKLFA-Z (SEQ ID NO: 5538); X-LKR-C(Sx)-AS*FKNFA-Z (SEQ ID NO: 5539); X-LKR-C(Sx)-AS*FKPFA-Z (SEQ ID NO: 5540); X-LKR-C(Sx)-AS*FKQFA-Z (SEQ ID NO: 5541); X-LKR-C(Sx)-AS*FKRFA-Z (SEQ ID NO: 5542); X-LKR-C(Sx)-AS*FKVFA-Z (SEQ ID NO: 5543); X-LKR-C(Sx)-AS*FKWFA-Z (SEQ ID NO: 5544); X-LKR-C(Sx)-AS*FLKFA-Z (SEQ ID NO: 5545); X-LKR-C(Sx)-AS*FNKFA-Z (SEQ ID NO: 5546); X-LKR-C(Sx)-AS*FPKFA-Z (SEQ ID NO: 5547); X-LKR-C(Sx)-AS*FQKFA-Z (SEQ ID NO: 5548); X-LKR-C(Sx)-AS*FRKFA-Z (SEQ ID NO: 5549); X-LKR-C(Sx)-AS*FVKFA-Z (SEQ ID NO: 5550); X-LKR-C(Sx)-AS*FWKFA-Z (SEQ ID NO: 5551); X-LKR-C(Sx)-AS*GKKFA-Z (SEQ ID NO: 5552); X-LKR-C(Sx)-AS*HKKFA-Z (SEQ ID NO: 5553); X-LKR-C(Sx)-AS*IKKFA-Z (SEQ ID NO: 5554); X-LKR-C(Sx)-AS*KKKFA-Z (SEQ ID NO: 5555); X-LKR-C(Sx)-AS*LKKFA-Z (SEQ ID NO: 5556); X-LKR-C(Sx)-AS*NKKFA-Z (SEQ ID NO: 5557); X-LKR-C(Sx)-AS*PKKFA-Z (SEQ ID NO: 5558); X-LKR-C(Sx)-AS*QKKFA-Z (SEQ ID NO: 5559); X-LKR-C(Sx)-AS*RKKFA-Z (SEQ ID NO: 5560); X-LKR-C(Sx)-AS*VKKFA-Z (SEQ ID NO: 5561); X-LKR-C(Sx)-AS*WKKFA-Z (SEQ ID NO: 5562); X-LKR-C(Sx)-DS*FKKFA-Z (SEQ ID NO: 5563); X-LKR-C(Sx)-ES*FKKFA-Z (SEQ ID NO: 5564); X-LKR-C(Sx)-FS*FKKFA-Z (SEQ ID NO: 5565); X-LKR-C(Sx)-GS*FKKFA-Z (SEQ ID NO: 5566); wherein X— is H or acetyl; and Z is OH or NH2.

In some embodiments, the metal-binding compound of the present invention is selected from the group consisting of: X-LKR-C(Sx)-HS*FKKFA-Z (SEQ ID NO: 5567); X-LKR-C(Sx)-IS*FKKFA-Z (SEQ ID NO: 5568); X-LKR-C(Sx)-KS*FKKFA-Z (SEQ ID NO: 5569); X-LKR-C(Sx)-LS*FKKFA-Z (SEQ ID NO: 5570); X-LKR-C(Sx)-NS*FKKFA-Z (SEQ ID NO: 5571); X-LKR-C(Sx)-PS*FKKFA-Z (SEQ ID NO: 5572); X-LKR-C(Sx)-QS*FKKFA-Z (SEQ ID NO: 5573); X-LKR-C(Sx)-RS*FKKFA-Z (SEQ ID NO: 5574); X-LKR-C(Sx)-VS*FKKFA-Z (SEQ ID NO: 5575); X-LKR-C(Sx)-WS*FKKFA-Z (SEQ ID NO: 5576); X-LKV-C(Sx)-AS*FKKFA-Z (SEQ ID NO: 5577); X-LKW-C(Sx)-AS*FKKFA-Z (SEQ ID NO: 5578); X-LLR-C(Sx)-AS*FKKFA-Z (SEQ ID NO: 5579); X-LNR-C(Sx)-AS*FKKFA-Z (SEQ ID NO: 5580); X-LPR-C(Sx)-AS*FKKFA-Z (SEQ ID NO: 5581); X-LQR-C(Sx)-AS*FKKFA-Z (SEQ ID NO: 5582); X-LRR-C(Sx)-AS*FKKFA-Z (SEQ ID NO: 5583); X-LVR-C(Sx)-AS*FKKFA-Z (SEQ ID NO: 5584); X-LWR-C(Sx)-AS*FKKFA-Z (SEQ ID NO: 5585); X-NKR-C(Sx)-AS*FKKFA-Z (SEQ ID NO: 5586); X-PKR-C(Sx)-AS*FKKFA-Z (SEQ ID NO: 5587); X-QKR-C(Sx)-AS*FKKFA-Z (SEQ ID NO: 5588); X-RKR-C(Sx)-AS*FKKFA-Z (SEQ ID NO: 5589); X-VKR-C(Sx)-AS*FKKFA-Z (SEQ ID NO: 5590); X-WKR-C(Sx)-AS*FKKFA-Z (SEQ ID NO: 5591); X-C(Sx)-PGS*FRR-Z (SEQ ID NO: 5592); X-C(Sx)-AS*FRR-Z (SEQ ID NO: 5593); X-C(Sx)-[Nle]GS*FRRR-Z (SEQ ID NO: 5594); X-C(Sx)-AGS*FRRR-Z (SEQ ID NO: 5595); X-C(Sx)-DGS*FRRR-Z (SEQ ID NO: 5596); X-C(Sx)-EGS*FRRR-Z (SEQ ID NO: 5597); X-C(Sx)-FGS*FRRR-Z (SEQ ID NO: 5598); X-C(Sx)-GGS*FRRR-Z (SEQ ID NO: 5599); X-C(Sx)-HGS*FRRR-Z (SEQ ID NO: 5600); wherein X— is H or acetyl; and Z is OH or NH2.

In some embodiments, the metal-binding compound of the present invention is selected from the group consisting of: X-C(Sx)-IGS*FRRR-Z (SEQ ID NO: 5601); X-C(Sx)-KGS*FRRR-Z (SEQ ID NO: 5602); X-C(Sx)-LGS*FRRR-Z (SEQ ID NO: 5603); X-C(Sx)-NGS*FRRR-Z (SEQ ID NO: 5604); X-C(Sx)-P[Nle]S*FRRR-Z (SEQ ID NO: 5605); X-C(Sx)-PAS*FRRR-Z (SEQ ID NO: 5606); X-C(Sx)-PDS*FRRR-Z (SEQ ID NO: 5607); X-C(Sx)-PES*FRRR-Z (SEQ ID NO: 5608); X-C(Sx)-PFS*FRRR-Z (SEQ ID NO: 5609); X-C(Sx)-PGS*[Nle]RRR-Z (SEQ ID NO: 5610); X-C(Sx)-PGS*ARRR-Z (SEQ ID NO: 5611); X-C(Sx)-PGS*DRRR-Z (SEQ ID NO: 5612); X-C(Sx)-PGS*ERRR-Z (SEQ ID NO: 5613); X-C(Sx)-PGS*F[Nle]RR-Z (SEQ ID NO: 5614); X-C(Sx)-PGS*FARR-Z (SEQ ID NO: 5615); X-C(Sx)-PGS*FDRR-Z (SEQ ID NO: 5616); X-C(Sx)-PGS*FERR-Z (SEQ ID NO: 5617); X-C(Sx)-PGS*FFRR-Z (SEQ ID NO: 5618); X-C(Sx)-PGS*FGRR-Z (SEQ ID NO: 5619); X-C(Sx)-PGS*FHRR-Z (SEQ ID NO: 5620); X-C(Sx)-PGS*FIRR-Z (SEQ ID NO: 5621); X-C(Sx)-PGS*FKRR-Z (SEQ ID NO: 5622); X-C(Sx)-PGS*FLRR-Z (SEQ ID NO: 5623); X-C(Sx)-PGS*FNRR-Z (SEQ ID NO: 5624); X-C(Sx)-PGS*FPRR-Z (SEQ ID NO: 5625); X-C(Sx)-PGS*FQRR-Z (SEQ ID NO: 5626); X-C(Sx)-PGS*FR[Nle]R-Z (SEQ ID NO: 5627); X-C(Sx)-PGS*FRAR-Z (SEQ ID NO: 5628); X-C(Sx)-PGS*FRDR-Z (SEQ ID NO: 5629); X-C(Sx)-PGS*FRER-Z (SEQ ID NO: 5630); X-C(Sx)-PGS*FRFR-Z (SEQ ID NO: 5631); X-C(Sx)-PGS*FRGR-Z (SEQ ID NO: 5632); X-C(Sx)-PGS*FRHR-Z (SEQ ID NO: 5633); X-C(Sx)-PGS*FRIR-Z (SEQ ID NO: 5634); X-C(Sx)-PGS*FRKR-Z (SEQ ID NO: 5635); X-C(Sx)-PGS*FRLR-Z (SEQ ID NO: 5636); wherein X— is H or acetyl; and Z is OH or NH2.

In some embodiments, the metal-binding compound of the present invention is selected from the group consisting of: X-C(Sx)-PGS*FRNR-Z (SEQ ID NO: 5637); X-C(Sx)-PGS*FRPR-Z (SEQ ID NO: 5638); X-C(Sx)-PGS*FRQR-Z (SEQ ID NO: 5639); X-C(Sx)-PGS*FRR[Nle]-Z (SEQ ID NO: 5640); X-C(Sx)-PGS*FRRA-Z (SEQ ID NO: 5641); X-C(Sx)-PGS*FRRD-Z (SEQ ID NO: 5642); X-C(Sx)-PGS*FRRE-Z (SEQ ID NO: 5643); X-C(Sx)-PGS*FRRF-Z (SEQ ID NO: 5644); X-C(Sx)-PGS*FRRG-Z (SEQ ID NO: 5645); X-C(Sx)-PGS*FRRH-Z (SEQ ID NO: 5646); X-C(Sx)-PGS*FRRI-Z (SEQ ID NO: 5647); X-C(Sx)-PGS*FRRK-Z (SEQ ID NO: 5648); X-C(Sx)-PGS*FRRL-Z (SEQ ID NO: 5649); X-C(Sx)-PGS*FRRN-Z (SEQ ID NO: 5650); X-C(Sx)-PGS*FRRP-Z (SEQ ID NO: 5651); X-C(Sx)-PGS*FRRQ-Z (SEQ ID NO: 5652); X-C(Sx)-PGS*FRRR-Z (SEQ ID NO: 5653); X-C(Sx)-PGS*FRRV-Z (SEQ ID NO: 5654); X-C(Sx)-PGS*FRRW-Z (SEQ ID NO: 5655); X-C(Sx)-PGS*FRVR-Z (SEQ ID NO: 5656); X-C(Sx)-PGS*FRWR-Z (SEQ ID NO: 5657); X-C(Sx)-PGS*FVRR-Z (SEQ ID NO: 5658); X-C(Sx)-PGS*FWRR-Z (SEQ ID NO: 5659); X-C(Sx)-PGS*GRRR-Z (SEQ ID NO: 5660); X-C(Sx)-PGS*HRRR-Z (SEQ ID NO: 5661); X-C(Sx)-PGS*IRRR-Z (SEQ ID NO: 5662); X-C(Sx)-PGS*KRRR-Z (SEQ ID NO: 5663); X-C(Sx)-PGS*LRRR-Z (SEQ ID NO: 5664); X-C(Sx)-PGS*NRRR-Z (SEQ ID NO: 5665); X-C(Sx)-PGS*PRRR-Z (SEQ ID NO: 5666); X-C(Sx)-PGS*QRRR-Z (SEQ ID NO: 5667); X-C(Sx)-PGS*RRRR-Z (SEQ ID NO: 5668); X-C(Sx)-PGS*VRRR-Z (SEQ ID NO: 5669); X-C(Sx)-PGS*WRRR-Z (SEQ ID NO: 5670); X-C(Sx)-PHS*FRRR-Z (SEQ ID NO: 5671); X-C(Sx)-PIS*FRRR-Z (SEQ ID NO: 5672); X-C(Sx)-PKS*FRRR-Z (SEQ ID NO: 5673); X-C(Sx)-PLS*FRRR-Z (SEQ ID NO: 5674); X-C(Sx)-PNS*FRRR-Z (SEQ ID NO: 5675); wherein X— is H or acetyl; and Z is OH or NH2.

In some embodiments, the metal-binding compound of the present invention is selected from the group consisting of: X-C(Sx)-PPS*FRRR-Z (SEQ ID NO: 5676); X-C(Sx)-PQS*FRRR-Z (SEQ ID NO: 5677); X-C(Sx)-PRS*FRRR-Z (SEQ ID NO: 5678); X-C(Sx)-PVS*FRRR-Z (SEQ ID NO: 5679); X-C(Sx)-QGS*FRRR-Z (SEQ ID NO: 5680); X-C(Sx)-RGS*FRRR-Z (SEQ ID NO: 5681); X-C(Sx)-VGS*FRRR-Z (SEQ ID NO: 5682); X-C(Sx)-[Nle]S*FRRR-Z (SEQ ID NO: 5683); X-C(Sx)-AS*FRRR-Z (SEQ ID NO: 5684); X-C(Sx)-DS*FRRR-Z (SEQ ID NO: 5685); X-C(Sx)-ES*FRRR-Z (SEQ ID NO: 5686); X-C(Sx)-FS*FRRR-Z (SEQ ID NO: 5687); X-C(Sx)-GS*[Nle]RRR-Z (SEQ ID NO: 5688); X-C(Sx)-GS*ARRR-Z (SEQ ID NO: 5689); X-C(Sx)-GS*DRRR-Z (SEQ ID NO: 5690); X-C(Sx)-GS*ERRR-Z (SEQ ID NO: 5691); X-C(Sx)-GS*F[Nle]RR-Z (SEQ ID NO: 5692); X-C(Sx)-GS*FARR-Z (SEQ ID NO: 5693); X-C(Sx)-GS*FDRR-Z (SEQ ID NO: 5694); X-C(Sx)-GS*FERR-Z (SEQ ID NO: 5695); X-C(Sx)-GS*FFRR-Z (SEQ ID NO: 5696); X-C(Sx)-GS*FGRR-Z (SEQ ID NO: 5697); X-C(Sx)-GS*FHRR-Z (SEQ ID NO: 5698); X-C(Sx)-GS*FIRR-Z (SEQ ID NO: 5699); X-C(Sx)-GS*FKRR-Z (SEQ ID NO: 5700); X-C(Sx)-GS*FLRR-Z (SEQ ID NO: 5701); X-C(Sx)-GS*FNRR-Z (SEQ ID NO: 5702); X-C(Sx)-GS*FPRR-Z (SEQ ID NO: 5703); X-C(Sx)-GS*FQRR-Z (SEQ ID NO: 5704); X-C(Sx)-GS*FR[Nle]R-Z (SEQ ID NO: 5705); X-C(Sx)-GS*FRAR-Z (SEQ ID NO: 5706); X-C(Sx)-GS*FRDR-Z (SEQ ID NO: 5707); X-C(Sx)-GS*FRER-Z (SEQ ID NO: 5708); X-C(Sx)-GS*FRFR-Z (SEQ ID NO: 5709); wherein X— is H or acetyl; and Z is OH or NH2.

In some embodiments, the metal-binding compound of the present invention is selected from the group consisting of: X-C(Sx)-GS*FRGR-Z (SEQ ID NO: 5710); X-C(Sx)-GS*FRHR-Z (SEQ ID NO: 5711); X-C(Sx)-GS*FRIR-Z (SEQ ID NO: 5712); X-C(Sx)-GS*FRKR-Z (SEQ ID NO: 5713); X-C(Sx)-GS*FRLR-Z (SEQ ID NO: 5714); X-C(Sx)-GS*FRNR-Z (SEQ ID NO: 5715); X-C(Sx)-GS*FRPR-Z (SEQ ID NO: 5716); X-C(Sx)-GS*FRQR-Z (SEQ ID NO: 5717); X-C(Sx)-GS*FRR[Nle]-Z (SEQ ID NO: 5718); X-C(Sx)-GS*FRRA-Z (SEQ ID NO: 5719); X-C(Sx)-GS*FRRD-Z (SEQ ID NO: 5720); X-C(Sx)-GS*FRRE-Z (SEQ ID NO: 5721); X-C(Sx)-GS*FRRF-Z (SEQ ID NO: 5722); X-C(Sx)-GS*FRRG-Z (SEQ ID NO: 5723); X-C(Sx)-GS*FRRH-Z (SEQ ID NO: 5724); X-C(Sx)-GS*FRRI-Z (SEQ ID NO: 5725); X-C(Sx)-GS*FRRK-Z (SEQ ID NO: 5726); X-C(Sx)-GS*FRRL-Z (SEQ ID NO: 5727); X-C(Sx)-GS*FRRN-Z (SEQ ID NO: 5728); X-C(Sx)-GS*FRRP-Z (SEQ ID NO: 5729); X-C(Sx)-GS*FRRQ-Z (SEQ ID NO: 5730); X-C(Sx)-GS*FRRR-Z (SEQ ID NO: 5731); X-C(Sx)-GS*FRRV-Z (SEQ ID NO: 5732); X-C(Sx)-GS*FRRW-Z (SEQ ID NO: 5733); X-C(Sx)-GS*FRVR-Z (SEQ ID NO: 5734); X-C(Sx)-GS*FRWR-Z (SEQ ID NO: 5735); X-C(Sx)-GS*FVRR-Z (SEQ ID NO: 5736); X-C(Sx)-GS*FWRR-Z (SEQ ID NO: 5737); X-C(Sx)-GS*GRRR-Z (SEQ ID NO: 5738); X-C(Sx)-GS*HRRR-Z (SEQ ID NO: 5739); X-C(Sx)-GS*IRRR-Z (SEQ ID NO: 5740); X-C(Sx)-GS*KRRR-Z (SEQ ID NO: 5741); X-C(Sx)-GS*LRRR-Z (SEQ ID NO: 5742); X-C(Sx)-GS*NRRR-Z (SEQ ID NO: 5743); X-C(Sx)-GS*PRRR-Z (SEQ ID NO: 5744); X-C(Sx)-GS*QRRR-Z (SEQ ID NO: 5745); X-C(Sx)-GS*RRRR-Z (SEQ ID NO: 5746); X-C(Sx)-GS*VRRR-Z (SEQ ID NO: 5747); X-C(Sx)-GS*WRRR-Z (SEQ ID NO: 5748); X-C(Sx)-HS*FRRR-Z (SEQ ID NO: 5749); X-C(Sx)-IS*FRRR-Z (SEQ ID NO: 5750); wherein X— is H or acetyl; and Z is OH or NH2.

In some embodiments, the metal-binding compound of the present invention is selected from the group consisting of: X-C(Sx)-KS*FRRR-Z (SEQ ID NO: 5751); X-C(Sx)-NS*FRRR-Z (SEQ ID NO: 5752); X-C(Sx)-PS*FRRR-Z (SEQ ID NO: 5753); X-C(Sx)-QS*FRRR-Z (SEQ ID NO: 5754); X-C(Sx)-RS*FRRR-Z (SEQ ID NO: 5755); X-C(Sx)-VS*FRRR-Z (SEQ ID NO: 5756); X-[Nle]-C(Sx)-GS*FRRR-Z (SEQ ID NO: 5757); X-A-C(Sx)-GS*FRRR-Z (SEQ ID NO: 5758); X-D-C(Sx)-GS*FRRR-Z (SEQ ID NO: 5759); X-E-C(Sx)-GS*FRRR-Z (SEQ ID NO: 5760); X-F-C(Sx)-GS*FRRR-Z (SEQ ID NO: 5761); X-G-C(Sx)-GS*FRRR-Z (SEQ ID NO: 5762); X-H-C(Sx)-GS*FRRR-Z (SEQ ID NO: 5763); X-I-C(Sx)-GS*FRRR-Z (SEQ ID NO: 5764); X-K-C(Sx)-GS*FRRR-Z (SEQ ID NO: 5765); X-L-C(Sx)-GS*FRRR-Z (SEQ ID NO: 5766); X-N-C(Sx)-GS*FRRR-Z (SEQ ID NO: 5767); X-Q-C(Sx)-GS*FRRR-Z (SEQ ID NO: 5768); X-R-C(Sx)-GS*FRRR-Z (SEQ ID NO: 5769); X-V-C(Sx)-GS*FRRR-Z (SEQ ID NO: 5770); X-P-C(Sx)-GS*FRRR-Z (SEQ ID NO: 5771); X-LD-C(Sx)-AS*LSLSLSSANSLYDD-Z (SEQ ID NO: 5772); X-SGDED-C(Sx)-AS*IADMDFSAL-Z (SEQ ID NO: 5773); X-SGDEDFAS*I-C(Sx)-DMDFSALLSQ-Z (SEQ ID NO: 5774); X-EDFAS*I-C(Sx)-DMDFSALLSQISS (SEQ ID NO: 5775); X-SLDSSSLAL-C(Sx)-LS*AANSLYDD-Z (SEQ ID NO: 5776); X-SLDSSSLS*L-C(Sx)-LASANSLYDD-Z (SEQ ID NO: 5777); X-DLGYAKDVDQGS*L-C(Sx)-TSFVGTLQY-Z (SEQ ID NO: 5778); X-RRKDLHDDEEDEA[Nle]S*I-C(Sx)-A-Z (SEQ ID NO: 5779); X-KKKVSGQLIDS*[Nle]-C(Sx)-NSFVGTRSY-Z (SEQ ID NO: 3646); X-KKKVSGQLIDS[Nle]-C(Sx)-NS*FVGTRSY-Z (SEQ ID NO: 3656); wherein X— is H or acetyl; and Z is OH or NH2.

In some embodiments, C(Sx) represents an amino acid of formula (I):

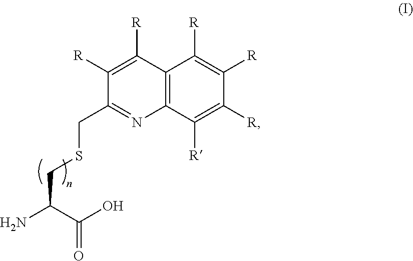

each R is independently hydrogen, or —SO₂X′, wherein at least one R group is —SO₂X′;

### X′ is —OR″ or —NR′R′″;

R′ is hydroxyl, amino, or thiol;
R″ is C₁₋₆ alkyl;
R′″ is hydrogen or alkyl; and
n is 1, 2 or 3.

In some embodiments, R′ is hydroxyl.

In some embodiments, at least one R is hydrogen. In some embodiments, all instances of R represent hydrogen.

In some embodiments, one and only one R is —SO₂X′.

In some embodiments, n is 1.

In some embodiments, R′ is hydroxyl and one and only one R is —SO₂X′.

In some embodiments, C(Sx) represents an amino acid of formula (II):

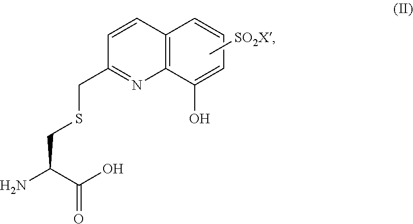

wherein

### X′ is —OR″ or —NR″R′″;

R″ is C₁₋₆ alkyl; and
R′″ is hydrogen or C₁₋₆ alkyl.

In some embodiments. C(Sx) represents an amino acid of formula (III):

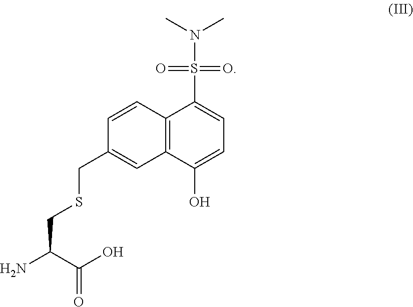 [SMILES: CN(C)S(=O)(=O)c1ccc(O)c2cc(CSC[C@H](N)C(=O)O)ccc12]

The metal-binding compounds of the present invention undergo chelation-enhanced fluorescence (CHEF) upon binding to Mg²⁺. The fluorescence of the amino acid residues in accordance with the present invention increases by at least about 100%, when bound to Mg²⁺. In some embodiments, fluorescence of the amino acid residues increases by at least about 200%, when bound to Mg²⁺. In some embodiments, fluorescence of the amino acid residues increases by at least about 400%, when bound to Mg²⁺.

The compounds of the invention can be synthesized using peptide synthesis (solid phase or solution phase). Standard peptide synthesis is well-known in the art. See, for example, Fmoc Solid Phase Peptide Synthesis—A Practical Approach, Oxford University Press, 2003, Eds W. C. Chan and P. D. White (ISBN 0 19 963 724 5); and The Chemical Synthesis of Peptides, Clarendon Press, Oxford, 1994, Jones, J. (ISBN 0 19 855839 2).

In some embodiments, the metal binding amino acid residue (e.g., —C(Sx)-) of the chemosensors is −2 or +2 residues from the amino acid (e.g., tyrosine, serine or threonine) that is phosphorylated by the kinase. In some embodiments, the metal binding amino acid residue of the chemosensors is −3 or +3 residues from the amino acid that is phosphorylated by the kinase. Negative (−) positions indicate the compound is located on the N-terminal side of the amino acid that is phosphorylated by the kinase, while positive (+) positions indicate the compound is located on the C-terminal side of the amino acid that is phosphorylated by the kinase.

The metal binding amino acid residue comprises a fluorophore that can be used as readout when positioned appropriately within the optimized peptide substrate to create fluorescence-based kinase assays. Examples of fluorophores include, but are not limited to, 5-FAM (5-Carboxyfluorescein; Caliper EZ Reader, Perkin Elmer and IMAP from Molecular Devices), Coumarin and Fluorescein (Z-Lyte, Thermo Fisher), pyrene, perylene, borodiazaindacene (BODIPY™), cyanine dye 2, cyanine dye 3, cyanine dye 5 and Alexa dyes.

In some embodiments, the metal binding amino acid residue is an amino acid of formula I, II, or III.

In some embodiments, the metal binding amino acid residue is Sox, C-Sox, or C-Clk.

In some embodiments, the metal binding amino acid residue is C-Sox.

Phosphorylation sites in accordance with the present invention include hydroxyl-containing amino acids within kinase recognition motifs. Examples include naturally occurring hydroxyl-containing amino acid residues, such as serine, threonine, tyrosine and histidine and non-naturally occurring hydroxyl-containing amino acid residues.

The peptide sensors according to the present invention have at least one kinase recognition sequence. In some cases, the residues on one side of the side chain to be phosphorylated are more important, however, it is clear that more residues might confer additional specificity. This specificity could play an important role in any assays that assess kinases in complex media, in particular in live cells or cell lysates where all cellular enzymes have the potential to interact with the substrate peptide. Added recognition elements can target the sensor more specifically to the desired kinase in competitive assays where several different kinases or isozymes of one kinase are present. Including additional amino acid sequence or altering the order of residues in the sequence can also improve the kinetic properties of the substrate, for example resulting in lower Km, higher Vmax or kcat etc. values.

### Exemplary Synthetic Procedures

The chemosensor peptides were constructed in a variety of ways.

In the one approach, Fmoc-based solid-phase peptide synthesis (SPPS) was utilized to assemble the intact peptide that includes an appropriately placed cysteine protected with an acid-labile group. After selective on-resin sulfhydryl group deprotection with acid, the free thiol is alkylated with Sox-Br. Standard TFA cleavage from the resin and concomitant removal of all side chain protecting groups reveal the desired chemosensor with excellent conversion to the alkylated product (>95%). The solid support-based alkylation is particularly valuable when utilizing automated SPPS or SPOT34 synthesis to generate libraries of peptides in a rapid manner.

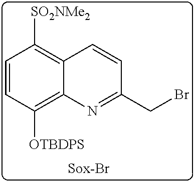

In some instances, peptides were synthesized on Fmoc-PAL-PEG-PS resin (Applied Biosystems, 0.19 mmol/g) using on-resin alkylation. The resin was swelled in CH₂Cl₂ (DCM) (5 min) and then DMF (5 min) prior to synthesis. The amino acids were coupled according to the following procedure: Fmoc deprotection (20% 4-methylpiperidine in DMF, 3-5 min), rinsing step (DMF), coupling step (amino acid/PyBOP/HOBt/DIEA, 6:6:6:6, 0.15 M in DMF, 30-45 min), rinsing step (DMF, then DCM). The coupling was repeated if necessary as determined by the TNBS test. At the end of the synthesis, the Fmoc group was removed with 20% 4-methylpiperidine in DMF, and the resin was rinsed with DMF. The resin-attached free amines were capped by exposure to Ac₂O (20 equiv.) and pyridine (20 equiv.) in DMF for 30 min. The resin was rinsed with DMF and then DCM, and subjected to 20% 4-methylpiperidine in DMF. The resin was finally washed with DMF, DCM, and MeOH, and dried under vacuum.

Resin-attached peptides (50 mg, 0.0095 mmol, 1 equiv) incorporating Cys-(Mmt) (Mmt=4-methoxytrityl) were swelled in DCM and then DMF. The Mmt protecting group was removed from the resin-bound peptide by bubbling N2 through a solution of 1% TFA and 5% TIS in DCM. The resin was washed with DCM and DMF. Anhydrous DMF was added to the resin followed by freshly distilled tetramethylguanidine (0.0475 mmol, 5 equiv.). The mixture was incubated for 2-3 min. Sox-Br (3 equiv.) was dissolved in anhydrous DMF and added to the resin. After ca. 12 h of reaction time, the excess reagents were drained and the resin was washed with DMF, DCM, MeOH, and DCM.

Alternatively, a building block approach may be used to prepare a chemosensor peptide. In this case, the synthesis of the building block, Fmoc-C(Sox[TBDPS])-OH, may commence with the allylation of commercially available amino acid, followed by removal of the p-methoxytrityl (Mmt) masking group. The sulfhydryl is then alkylated with Sox-Br in excellent yield (95%). Lastly, Pd(II)-assisted deallylation produces the desired amino acid that was subsequently used in standard Fmoc-based SPPS (described above) to produce sensors in excellent yields. Peptide identities and purities were confirmed via matrix-assisted laser desorption ionization time-of-flight (MALDI TOF) or electrospray ionization mass spectrometry together with high performance liquid chromatography analysis. The synthesis of C-Sox, and therefore of chemosensor peptides, is facile since a chiral starting material is used.

Peptides were purified by preparative reversed-phase HPLC using dual wavelength detection (228 nm: amide; 360 nm: C-Sox). Peptide concentrations were then determined using UV-vis spectrophotometry based on the C-Sox-chromophore extinction coefficient of 8247M-1 cm-1 at 355 nM in 0.1 M NaOH and 1 mM EDTA.

### Assays

Fluorescence of the peptides was measured in the presence and absence of Mg²⁺. The reporter functionality is the unnatural chromophore (e.g., C-Sox) that undergoes chelation-enhanced fluorescence (CHEF) upon metal binding. The Mg²⁺ affinity of the chemosensors is low (K=100-300 mM) when the phosphate group is not present, while phosphorylation significantly increases Mg²⁺ affinity (K_{D}=0.1-20 mM). Thus, at a selected Mg²⁺ concentration, a large portion of phosphopeptide exists in the bound, fluorescent state. In the presence of saturated MgCl₂, the Sox peptides exhibit a maximum emission at 485 nm with a maximum excitation at 360 nm. In addition, these peptides maintain striking luminescence properties of the fluorophore. The extinction coefficient and quantum yield values for representative chemosensor-Mg²⁺ complexes are determined following quantitative amino acid analysis.

To solve for enzyme kinetic parameters for this reaction, determination of the initial rate of product formation from the increase in fluorescence intensity is necessary. With this sensor, a correction for the decrease in fluorescence intensity due to the starting material being consumed is needed to determine the rate of product formation from the initial slope. The fluorescence intensity at any given point can be determined from the following equation:

I(t)=f_{S}s(t)+f_{P}P(t) (1)

where I(t) is the fluorescence intensity. S(t) is the amount of substrate in μM, P(t) is the amount of product in IM, f_{S} is the fluorescence intensity per μM of substrate, and f_{P} is fluorescence intensity per μM of product. The amount of substrate and product at any given point are related by:

S(t)+P(t)=S₀ (2)

where S₀ is the initial amount of substrate. Substitution of (2) into (1) followed by rearrangement, yields:

$$P ( t ) = \frac{I ( t ) - {f}_{S} {S}_{0}}{{f}_{P} - {f}_{S}} \qquad ( 3 )$$

The initial velocity of the reaction is the change in the amount of product over time, so taking the derivative of (3) with respect to time gives:

$$v = \frac{d P ( t )}{d t} = \frac{\frac{d I ( t )}{d t}}{{f}_{P} - {f}_{S}} \qquad ( 4 )$$

The initial slope of the reaction, dI(t)/dt, was measured within the first 5% of substrate turnover. The constants f_{P} and f_{S} were calculated from the slope of a line of fluorescence intensity versus concentration of P and S, respectively. A linear fit of a Hanes plot ([S]/V vs. V) was used to find Kₘ and Vₘₐₓ.

The sensors of the present invention can be used in a method for detecting kinase activity.

In some embodiments, the method for detecting kinase activity, comprising the steps of:

a) providing a compound of the invention;

b) contacting the compound with a sample comprising Mg²⁺, a phosphate source, and a kinase; and

c) analyzing for the presence of a phosphorylated peptide product:

In some embodiments, the method for detecting kinase activity, comprising the step of:

determining phosphorylation of a compound of the invention using fluorescence of the compound.

Serine/threonine and tyrosine kinases can be used in the present invention. Exemplary Ser/Thr kinases include cAMP dependent protein kinase, protein kinase C, Ca/calmodulin-dependent kinases, AMP activated kinase, s6 kinases, eIF-2 kinases, p34^{cdc2} protein kinase, mitogen-activated protein kinases, casein kinase-2, casein kinase-1, glycogen synthase kinase-3, AURORA, Akt, Erk, ink, CDK2, and exemplary Tyr-specific protein kinases include Src Abl, insulin receptor kinase and EGFR among others.

For in vitro applications, the concentration of kinase can range from about 0.5 nM to about 100 nM, typically not more than about 500 nM, and preferably not more than about 250 nM. The concentrations of sensor can vary, but usually ranges between about 0.01 μM to 0.1 mM. Adenosine 5′-triphosphate (ATP) is the preferred source of phosphate, in stock solutions of about 10-100 mM. Because most kinases have Kₘ values for ATP in the range of about 10-150 μM, saturating concentrations of ATP are used to arrive at values of Kₘ and Vₘₐₓ for the substrates. Since this assay format can be used with any ATP concentration, 1 mM or ˜physiological concentrations are used unless being titrated. For in vivo applications, when the sensor is internalized into a cell, sufficient kinases, Mg²⁺ and phosphate sources exist in the cytosol. For in vivo sensing, a cellular internalization sequence can be included in the sensor design. Suitable cellular internalization sequences include Penetratins. HIV-Tat domains and poly-arginine sequences (Lindgren, M. et al. Trends Pharmacol. Sci. 2000, 21, 99-103; Wadia, J. S. et al. Curr Opin. Biotech. 2002, 13, 52-56; Carrigan, C. N. Analyt. Biochem. 2005, 341, 290-298).

For applications in which the kinase is dependent on cofactors, a source of cofactor is also included in the sample. For example, for certain PKC family enzymes, sources of Ca²⁺, phospholipid and diacylglycerol are needed.

The sensors of the present invention can be used to measure a kinase reaction continuously, as the metal-binding amino acid residues do not experience photobleaching.

### EXAMPLES

General Experimental

A fluorescence-based assay may be performed to determine the activity of a given protein kinase. This involves incubating the protein kinase with a suitable fluorescent peptide substrate and the co-substrates in an appropriate buffer and monitoring the change in fluorescence over time. Generally, a reaction mixture is prepared in the absence of a substrate peptide, which is then added to initiate the assay. The assay may be performed in a fluorimeter, where usually a single reaction is monitored at a time, or in a plate reader where multiple reactions may be monitored simultaneously.

This experimental may be modified based on various optimized reaction conditions as detailed in Examples 1-22.

Microtiter plates can be 96-, 384- or 1536-well formats. The final reaction volume is based on the manufacturers recommendations for each plate product, which may also vary based on the type of experiment, for example, 25-125 uL for Corning 96-well Half Area Flat Bottom Polystyrene NBSTM Microplates (Cat #3642) or 5-50 uL for Corning 384-well Low Volume Flat Bottom Polystyrene NBSTM Microplates (Cat #3824).

The reactions in each plate are incubated at the specified temperature and the fluorescence signal is monitored in kinetic mode using an appropriate microplate reader with an excitation wavelength of 365 nm and an emission wavelength of 485 nm Somewhat lower or higher wavelengths can also be used as long as the resulting signal is sufficient. Readings are made at preset intervals (e.g., every 5-30 seconds) over the desired time period (typically 30 min to 2 hours). Although the assay is most commonly monitored kinetically, it is also possible to stop the reactions using 5M Guanidine Hydrochloride to read the reactions in end point mode.

Setting up a reaction initiated with the peptide substrate:

1. Prepare Solution A (Assay reaction mixture comprises all reagents but the Sox-based Peptide Substrate) and incubate on ice:
2. Assay Reaction mixture:
i. Add 40 μL of the reaction mixture to each well of the 96-well plate, pre-warmed at 30° C.
ii. Incubate reactions at 30° C. in the plate reader for 5 minutes.
iii. Initiate the reactions with 10 μL of the 5× peptide solution, pre-warmed to 30° C.
iv. Monitor the change in fluorescence emission continuously at 485 nm during the linear phase of the reaction (conversion of the first 5-10% of the substrate to product) where readings are taken every 5-30 seconds (most plate readers have a setting that allows measurements to be determined at set time intervals).

Alternatively, solution B (Assay reaction mixture comprises all reagents but the protein kinase enzyme) or solution C (Assay reaction mixture comprises all reagents but the ATP) could be used and the reactions would be initiated in step 4 by addition of the enzyme or ATP respectively.

### Example 1: Receptor Tyrosine Kinase Activity

### Reaction Conditions:

### 50 mM HEPES, pH 7.4

### 10 mM MgCl₂

### 0.5 mM EGTA

### 10 μM Sox-based Peptide Substrate

### 1 mM ATP

### 1 mM DTT

### 0.01% Brij-35

5 nM Carna Bioscience kinase (or alternative enzyme sources)
30 minute reaction at 30° C.

### * +3 to 300 μM MnCl₂

* Used for kinases that require MnCl₂

### Receptor-type Tyrosine Kinases:

### ALK (cat #08-518, lot #09CBS-0698F)

### EML4-ALK (cat #08-516, lot #09CBS-0483D

### NPM1-ALK (cat #08-517, lot #08CBS-1302D)

### ALK (G1269A) (cat #08-537, lot #12CBS-0530B)

### ALK (T1151_L1152 ins T)(cat #08-539, lot #12CBS-0810B)

### AXL (cat #08-107, lot #09CBS-0686J)

### EGFR (cat #08-115, lot #13CBS-0005P)*

EGFR (d746-750) (cat #08-527, lot #11CBS-0259E)*
EGFR (d746-750/T790M) (cat #08-528, lot #11CBS-0285H)*

### EGFR (L858R) (cat #08-502, lot #09CBS-0597E)*

### EGFR (T790M′L858R) (cat #08-510, lot #11CBS-0004B)*

### EPHA1 (cat #08-119, lot #10CBS-0048B)

### EPHA2 (cat #08-121, lot #08CBS-0480L)

### EPHA4 (cat #08-123, lot #08CBS-1014B)

### EPHA7 (cat #08-126, lot #07CBS-2615J)

### EPHA8 (cat #08-127, lot #07CBS-1584G)

### EPHB1 (cat #08-128, lot #09CBS-1144B)

### EPHB2 (cat #08-129, lot #07CBS-2306G)

### EPHB3 (cat #08-130, lot #06CBS-3285L)

### EPHB4 (cat #08-131, lot #07CBS-3269F)

### FGFR1 (cat #08-133, lot #12CBS-0123J)

### FGFR2 (cat #08-134, lot #07CBS-2468F)

### FGFR3 (cat #08-135, lot #12CBS-0744K)

### FGFR3 (K650E) (cat #08-501, lot #07CBS-1740H)

### FGFR3 (G697C) (cat #10-1-0000-1, lot #001)*

### FGFR4 (cat #08-136, lot #12CBS-0076F)

### FLT1 (cat #08-189, lot #09CBS-0092E)

### FLT4 (cat #08-190, lot #13CBS-0858B)

### HER2 (cat #08-016, lot #10CBS-0748T)*

### HER4 (cat #08-118, lot #08CBS-0652P)

### IGF1R (cat #08-141, lot #07CBS-0753B)

### INSR (cat #08-142, lot #07CBS-0620K)

### KDR (cat #08-191, lot #13CBS-0442G)

### KIT (cat #08-156, lot #11CBS-1067L)

### LTK (cat #08-106, lot #06CBS-2044L)

### MET (cat #08-151, lot #10CBS-1118N)

### MUSK (cat #08-153, lot #07CBS-0004J)*

### PDGFRα (cat #08-157, lot #009BS-1164B)

### PDGFRβ (cat #08-158, lot #08CBS-0282H)

### RET (cat #08-159, lot #13CBS-0134B)

### RON (cat #08-152, lot #08CBS-0903B)

### TIE2 (cat #08-185, lot #07CBS-2698H)

### TRKA (cat #08-186, lot #13CBS-0565M)

### TRKB (cat #08-187, lot #09CBS-0265G)

### TRKC (cat #08-197, lot #08CBS-0549F)

*Indicated enzyme obtained from ProQinase (other sources of kinases have also been validated)

### Example 2: Receptor Tyrosine Kinase Activity

### Reaction Conditions:

### 20 mM HEPES, pH 7.4

### 5 mM MgCl₂

### 0.1 mM EGTA

### 10 μM Sox-based Peptide Substrate

### 1 mM ATP

### 0.2 mM DTT

### 0.01% Brij-35

5 nM Carna Bioscience kinase

### Example 3: CMGC Serine/Threonine Kinase Activity

### Reaction Conditions:

### 50 mM HEPES, pH 7.4

### 10 mM MgCl₂

### 0.5 mM EGTA

### 10 μM Sox-based Peptide Substrate

### 1 mM ATP

### 1 mM DTT

### 0.01% Brij-35

5 nM Carna Bioscience kinase

### CMGC Serine Threonine Kinases:

### DYRK1A (cat #04-130, lot #10CBS-0742H)

### DYRK1B (cat #04-131, lot #13CBS-0831D)

### ERK1 (cat #04-142, lot #11CBS-0072E)

### ERK2 (cat #04-143, lot #10CBS-0740E)

### ERK5 (cat #04-146, lot #13CBS-0805B)

### ERK7 (cat #04-147, lot #09CBS-0079E)

### JNK1 (cat #04-163, lot #09CBS-0154B)

### JNK2 (cat #04-164, lot #07CBS-1214G)

### JNK3 (cat #04-150, lot #09CBS-0155G)

p38α (cat #04-152, lot #07CBS-0319H)
p38β (cat #04-153, lot #07CBS-0172D)
p38γ (cat #04-155, lot #07CBS-2630B)
p38δ (cat #04-154, lot #07CBS-2631B)

### MK2 (cat #07-142, lot #10CBS-0101B)

### Example 4: AGC Serine/Threonine Kinase Activity AQT0074 (Ac-ARKRERAYSF-C(Sx)-HHA-amide)

### Reaction Conditions:

### 50 mM HEPES, pH 7.4

### 10 mM MgCl₂

### 0.5 mM EGTA

### 10 μM Sox-based Peptide Substrate

### 1 mM ATP

### 1 mM DTT

### 0.01% Brij-35

5 nM Carna Bioscience kinase

### *20 mM HEPES, pH 7.4

### 10 mM MgCl₂

### 0.1 mM EGTA

### 10 μM Peptide

### 0.1 mM ATP

### 1 mM DTT

### 0.01%0/Brij-35

### 0.3 mM CaCl₂

0.5 μg/ml phosphatidylserine
0.1 μg/ml diacylglycerol
5 nM Carna Bioscience kinase

### AGC Serine/Threonine Kinases:

### AKT1 (cat #01-101, lot #08CBS-0149J)

### AKT2 (cat #01-102, lot #07CBS-3271B)

### AKT3 (cat #01-103, lot #09CBS-0605G)

### CRIK (cat #01-104, lot #11CBS-1034B)

p70S6K (cat #01-176, lot #14CBS-0617B)

### PASK (cat #02-128, lot #07CBS-1805E)

### PHKG2 (cat #02-153, lot #10CBS-0646B)

### PKACα (cat #01-127, lot #10CBS-0315N)

### PKACγ (cat #01-129, lot #12CBS-0329B)

### PKCα (cat #01-133, lot #09CBS-0233H)*

### PKCδ (cat #01-135, lot #07CBS-2223B)*

### PKCγ (cat #01-137, lot #07CBS-2676F)*

### SGK (cat #01-158, lot #09CBS-0646F)

### SGK2 (cat #01-159, lot #08CBS-0830B)

### SGK3 (cat #01-160, lot #08CBS-0586B)

### RSK1 (cat #01-149, lot #08CBS-0354K)

### Example 5: ABL Substrate Time Course

### Reaction Conditions:

### 20 mM HEPES, pH 7.4

### 5 mM MgCl₂

### 1 mM ATP

### 1 mM DTT

### 0.01% Brij-35

### 3.3 nM Protein Kinase:

ABL, amino acids 2-1130 Carna Bioscience (cat #08-001, lot #14CBS-0812 E)
ARG, amino acids 2-52, 74-1182 Carna Bioscience (cat #08-102, lot #07CBS-1313 B)
ABL(T3151), amino acids 2-1130 Carna Bioscience (cat #08-093, lot #08CBS-1142 B)
ABL(E255K), amino acids 2-1130 Carna Bioscience (cat #08-094, lot #08CBS-0324 B)
PDGFRβ, amino acids 557-1106 Carna Bioscience (cat #08-158, lot #08CBS-0282 H)
KIT, amino acids 544-976 Carna Bioscience (cat #08-156, lot #11CBS-1067 L)

### 10 μM Sox-based Peptide Substrate:

### AQT0030 (Ac-KKGEAIYA-C(Sx)-PFAKKK-NH₂)

### AQT0031 (Ac-KKGE-C(Sx)-IYAAPFAKKK-NH₂)

### AQT0032 (Ac-E-C(Sx)-IYAAPFAKKK-NH₂)

### AQT0033 (Ac-GAPE-C(Sx)-IYATPGAKKK-NH₂)

### AQT0034 (Ac-GAPEVIYA-C(Sx)-PGAKKK-NH₂)

### AQT0035 (Ac-E-C(Sx)-IYAAPF-NH₂

### AQT0036 (Ac-E-C(Sx)-IYA-NH₂

### AQT0037 (Ac-E-C(Sx)-IY-NH₂)

### AQT0038 (Ac-C(Sx)-IY-NH₂)

### AQT0039 (Ac-KKGEAIYA-C(Sx)-PF-NH₂)

### Reaction was run at 30′C.

### Example 6: Soluble Tyrosine Kinase Activity

### Reaction Conditions:

### 20 mM HEPES, pH 7.4

### 5 mM MgCl₂

### 0.1 mM EGTA

### 10 μM Sox-based Peptide Substrate

### 1 mM ATP

### 0.2 mM DTT

### 0.01% Brij-35

5 nM Carna Bioscience kinase
30 minute reaction at 30° C.

### * +3-300 μM MnCl₂

### Soluble Tyrosine Kinases:

### ABL (cat #08-001, lot #14CBS-0812E)

### ABL (E255K) (cat #08-094, lot #08CBS-0324B)

### ABL (T3151) (cat #08-093, lot #11CBS-0498J)

### ACK (cat #08-196, lot #13CBS-0886B)

### ARG (cat #08-102, lot #07CBS-1313B)

### BLK (cat #08-164, lot #08CBS-1084B)

### BMX (cat #08-179, lot #08CBS-0426G)

### BRK (cat #08-165, lot #07CBS-2947D)

### BTK (cat #08-180, lot #14CBS-0619F)

### CSK (cat #08-111, lot #12CBS-0040E) *

### FAK (cat #08-137, lot #12CBS-0121E)

### FER (cat #08-139, lot #10CBS-0584B)

### FGR (cat #08-166, lot #10CBS-0459B)

### FRK (cat #08-167, lot #08CBS-1152G)

### FYN (cat #08-168, lot #11CBS-1043E)

### HCK (cat #08-169, lot #11CBS-0575B)

### ITK (cat #08-181, lot #13CBS-0356L)

### JAK1 (cat #08-144, lot #11CBS-0144T)

### JAK2 (cat #08-514, lot #08CBS-1002D)

### JAK3 (cat #08-046, lot #11CBS-0891E)

### LCK (cat #08-170, lot #10CBS-0924P)

### SRC (cat #08-173, lot #10CBS-1134F)

### SYK (cat #08-176, lot #13CBS-0926E)

### YES (cat #08-175, lot #09CBS-0412E)

### Example 7: EGFR Assay Optimization

Reaction Conditions (except where titrated):

### 20 mM HEPES, pH 7.4

### 1 mM ATP

### 1 mM DTT

### 0.01% Brij-35

### 0.1 mM EGTA

### 10 mM MgCl₂

### 3 μM MnCl₂

### 30 μM AQT0001 (Ac-EEEEYI-C(Sx)-IV-NH2) Sox-based Peptide Substrate

2 nM EGFR, amino acids 669-1210

### Carna Bioscience (cat. & lot #: 08-115, 13CBS-0005P)

Reaction was run at 30° C. for 2 hours

### Example 8: Screen of Tyr Kinase Substrates with Tec

### Reaction Conditions:

### 50 mM HEPES, pH 7.5

### 1 mM ATP

### 1 mM DTT

### 0.01% Brij-35

### 0.5 mM EGTA

### 10 mM MgCl₂

### 10 μM Sox-based Peptide Substrate

5 nM BTK, amino acids 2-659, Carna Bioscience (cat. & lot #: 08-080, 14CBS-0619F)
Reaction was run at 30′C for 60 minutes.

### Example 9: Serine/Threonine Kinase Activity Measured Using AQT's Panel of Substrates

### Reaction Conditions:

### 50 mM HEPES, pH 7.5

### 1 mM ATP

### 1 mM DTT

### 0.01% Brij-35

### 0.5 mM EGTA

### 10 mM MgCl₂

### 10 μM Sox-based Peptide Substrate

5 nM AKT1, amino acids 104-480, Carna Bioscience (cat. & lot #: 01-101, 08CBS-0149J)
5 nM AKT2, amino acids 120-481, Carna Bioscience (cat. & lot #: 01-102, 07CBS-3271B)
5 nM AKT3, amino acids 108-479, Carna Bioscience (cat. & lot #: 01-103, 09CBS-0605G)

### Reaction run at 30° C. for 60 min.

### Example 10: CHEK1 (CAMK Group) Serine/Threonine Kinase Activity

### Reaction Conditions:

### 50 mM HEPES, pH 7.5

### 1 mM ATP

### 1 mM DTT

### 0.01% Brij-35

### 0.5 mM EGTA

### 10 mM MgCl₂

10 μM CHK Sox-based peptide substrate
5 nM CHK1, amino acids 1-476, CARNA BS (cat. & lot #: 02-117, 08CBS-8008H)
Reaction was run at 30° C. for 120 minutes.

### Example 11: CHEK2 (CAMK Group) Serine/Threonine Kinase Activity

### Reaction Conditions:

### 50 mM HEPES, pH 7.5

### 1 mM ATP

### 1 mM DTT

### 0.01% Brij-35

### 0.5 mM EGTA

### 10 mM MgCl₂

### 10 μM CHK Sox-based Peptide Substrate

5 nM CHK2, amino acids 1-543, CARNA BS (cat. & lot #: 02-162, 10CBS-0386B)
Reaction was run at 30′C for 120 minutes.

### Example 12: Comparison of a Panel of CSx and Sox-Pro (Y4) RTK Substrates for TrkA

### Reaction Conditions:

### 50 mM HEPES, pH 7.5

### 1 mM ATP

### 1 mM DTT

### 0.01% Brij-35

### 0.5 mM EGTA

### 10 mM MgCl₂

### 10 μM Sox-based Peptide Substrate

5 nM* TRKA, amino acids 436-790

### Carna Bioscience (cat. & lot #: 08-186, 13CBS-0585M)

Reaction was run at 30° C. for 180 minutes.

### Example 13: EGFR Substrate Optimization (Mutant EGFRs)

### Reaction Conditions:

50 mM HEPES, pH 7.5 5% glycerol 1 mM ATP 1 mM DTT 0.01% Brij-35 0.1% CHAPS 0.5 mM EGTA 10 mM MgCl2 250 μM MnCl2 10 μM Sox-based Peptide Substrate 5 nM EGFR:
Wild-type, amino acids 668-1210. BPS BS (cat. & lot #: 40187, 120321-GC)
Mutant (L858R/T790M), amino acids 668-1210. BPS BS (cat. & lot #: 40350, 150804-G2)
Mutant (L858R/T790M), amino acids 669-1210, CARNA BS (cat. & lot #: 08-510, 11CBS-0004B)
Mutant (L861Q), amino acids 669-1210, CARNA BS (cat. & lot #: 08-513, 10CBS-0896B)
Mutant (T790M), amino acids 669-1210, CARNA BS (cat. & lot #: 08-522, 07CBS-0923B)
Mutant (L858R), amino acids 669-1210, CARNA BS (cat. & lot #: 08-502, 09CBS-0597E)
Mutant (d746-750), amino acids 669-745, 751-1210, CARNA BS (cat. & lot #: 08-527, 11CBS-0259E)
Mutant (d746-750T790M), amino acids 669-745, 751-1210, CARNA BS (cat. & lot #: 08-528, 11CBS-0285H) Reaction was run at 30′C for 120 minutes.

### Example 14: Broad AGC Serine/Threonine Kinase Activity Measured Using PKA-S4 Including SGK1/2/3 and PRKACA/B

### Reaction Conditions:

### 50 mM HEPES, pH 7.5

### 1 mM ATP

### 1 mM DTT

### 0.01% Brij-35

### 0.5 mM EGTA

### 10 mM MgCl₂

### 10 μM PKA-S4 Sox-based Peptide Substrate

5 nM kinase
Reaction was run at 30° C. for 120 minutes.

### Example 15: Substrates for SGK1, SGK2, SGK3 Identified from AQT's CSx Panel

### Reaction Conditions:

### 50 mM HEPES, pH 7.5

### 1 mM ATP

### 1 mM DTT

### 0.01% Brij-35

### 0.5 mM EGTA

### 10 mM MgCl₂

### 10 μM Sox-based Peptide Substrate

5 nM SGK, amino acids 61-431. Carna Bioscience (cat. & lot #: 01-158, 09CBS-0646F)
5 nM SGK2, amino acids 1-367, Carna Bioscience (cat. & lot #: 01-159, 08CBS-0830B)
5 nM SGK3, amino acids 119-496, Carna Bioscience (cat. & lot #: 01-160, 08CBS-0586B)
Reaction was run at 30′C for 120 minutes.

### Example 16: AQT's Panel of CSx and Sox-Pro (Y4) RTK Substrates for TrkA

### Reaction Conditions:

### 50 mM HEPES, pH 7.5

### 1 mM ATP

### 1 mM DTT

### 0.01% Brij-35

### 0.5 mM EGTA

### 10 mM MgCl₂

### 10 μM Sox-based Peptide Substrate

5 nM* TRKA, amino acids 436-790, Carna Bioscience (cat. & lot #: 08-186, 13CBS-0585M)
Reaction was run at 30° C. for 180 minutes.

### Example 17: Substrates for TRK RTKs

### Reaction Conditions:

### 50 mM HEPES, pH 7.5

### 1 mM ATP

### 1 mM DT

### 0.01% Brij-35

### 0.5 mM EGTA

### 10 mM MgCl₂

### 10 μM Sox-based Peptide Substrate

5 nM TRKA, amino acids 442-796, BPS BS (cat. & lot #: 40280, 151113-G)
5 nM TRKB, amino acids 456-822, BPS BS (cat. & lot #: 40281, 130826)
5 nM TRKC, amino acids 456-825. BPS BS (cat. & lot #: 40282, 140205-G1)
Reaction was run at 30° C. for 150 minutes.

### Example 18: FGFR Substrate Optimization (Mutant FGFRs)

### Reaction Conditions:

### 50 mM HEPES, pH 7.5

### 1 mM ATP

### 1 mM DTT

### 0.01% Brij-35

### 0.5 mM EGTA

### 10 mM MgCl₂

### 10 μM Sox-based Peptide Substrate

5 nM FGFR1, amino acids 396-820, CARNA BS (cat. & lot #: 08-133, 12CBS-0123J)
5 nM FGFR2, amino acids 399-821, CARNA BS (cat. & lot #: 08-134, 07CBS-2468F)
5 nM FGFR3, amino acids 436-806, CARNA BS (cat. & lot #: 08-135, 12CBS-0744K)
5 nM FGFR4, amino acids 460-802, CARNA BS (cat. & lot #: 08-136, 12CBS-0076F)
5 nM FGFR (K650E), amino acids 436-806, CARNA BS (cat. & lot #: 08-501, 07CBS-1740H)
5 nM FGFR (G697C), amino acids 397-806 Proqinase (cat. & lot #: 1071-0000-1, 001)
5 nM FGFR (K650M), amino acids 436-806, CARNA BS (cat. & lot #: 08-199, 08CBS-0670B)
5 nM FGFR (V555L), amino acids 436-806, CARNA BS (cat. & lot #: 08-548, 15CBS-0370B)
5 nM FGFR (V555M), amino acids 436-806, CARNA BS (cat. & lot #: 08-543, 12CBS-0565E)
Reaction was run at 30° C. for 90 minutes.

### Example 19: Fmoc-C(Sox[TBDPS])-OH Synthesis

a. Fmoc-Cys-OAlly

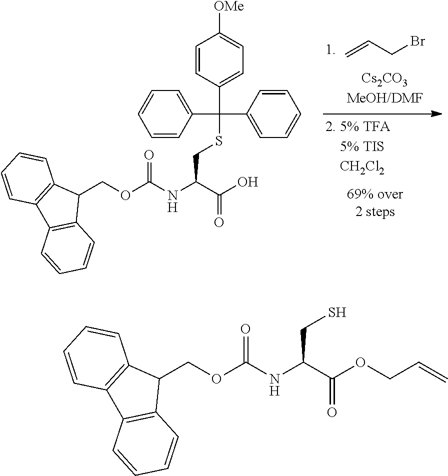

To an oven-dried, one-necked, 500 mL round-bottomed flask equipped with a stir bar and under positive N2 pressure was added Fmoc-Cys(Mmt)-OH (8.92 mmol, 5.49 g, 1 equiv. dissolved in 50 mL CH2Cl2) followed by MeOH (50 mL). The colorless solution then was allowed to stir for 3 min at room temperature. To this solution was added Cs₂CO₃ (4.46 mmol, 1.45 g, 0.5 equiv.) and mixture was allowed to stir at room temperature under N2 for 45 min at which time the solvent was removed in vacuo. The resulting white, fluffy solid was dissolved in DMF (100 mL, anhydrous) and immediately following this, allyl bromide (26.75 mmol, 3.24 g, 3 equiv.) was added. The reaction mixture was stirred at room temperature under positive N2 pressure for 5 hours. During this time, the reaction was monitored by TLC (Rf=0.23, 20% EtOAc in hexanes). Upon completion, the reaction mixture was diluted with EtOAc (1 L), washed with 2% NaHCO₃ (200 mL), H₂O (250 mL×3), brine (250 mL×3), dried (Na₂SO₄), and concentrated in vacuo.

The crude product from the previous step was dissolved in degassed, anhydrous CH2Cl2 (90 mL). To the slightly yellow solution were added TIS (5 mL) and TFA (5 mL). The resulting red solution was allowed to react at room temperature under N² for 2.5 hrs and was monitored by TLC (Rf=0.28, 20% EtOAc in hexanes). The reaction mixture turns a clear yellow color upon completion. The reaction then was diluted in CH2Cl2 (300 mL), washed with 5% NaHCO₃ (250 mL×2). H₂O (250 mL), brine (300 mL), dried (Na2SO4) and concentrated in vacuo. Flash column chromatography (SiO₂; diameter: 70 mm; length: 23 cm; packing: 20% EtOAc in hexanes; load crude product in CH2Cl2; eluent: 20% EtOAc in hexanes) was used to purify the product, which was isolated as a fluffy white solid. Yield: 69% (2.36 g), 1H NMR (400 MHz, CDCl₃, 20° C.) δ ppm: 1.38 (t, J=8.90 Hz, 1H), 3.10-2.94 (m, 2H), 4.24 (t, J=6.83 Hz, 1H), 4.49-4.38 (m, 2H), 4.78-4.62 (m, 3H), 5.29 (d, J=10.38 Hz, 1H), 5.37 (d, J=17.15 Hz, 1H), 5.72 (d, J=6.97 Hz, 1H), 5.98-5.88 (m, 1H), 7.33 (t, J=7.45 Hz, 2H), 7.41 (t, J=7.47 Hz, 2H), 7.61 (d, J=7.25 Hz, 2H), 7.77 (d, J=7.48 Hz, 2H), 13C NMR (100 MHz, CDCl₃, 20° C.) δ ppm: 27.1, 47.1, 55.2, 66.5, 67.1, 119.4, 120.0, 125.0, 127.1, 127.7, 131.2, 141.3, 143.7, 155.6, 169.6. HRMS (ESI): calcd for C21H21NNaO4S [M+Na]+: 406.1089, found: 406.1098.

b. Fmoc-Cys(Sox[TBDPS])-OAllyl

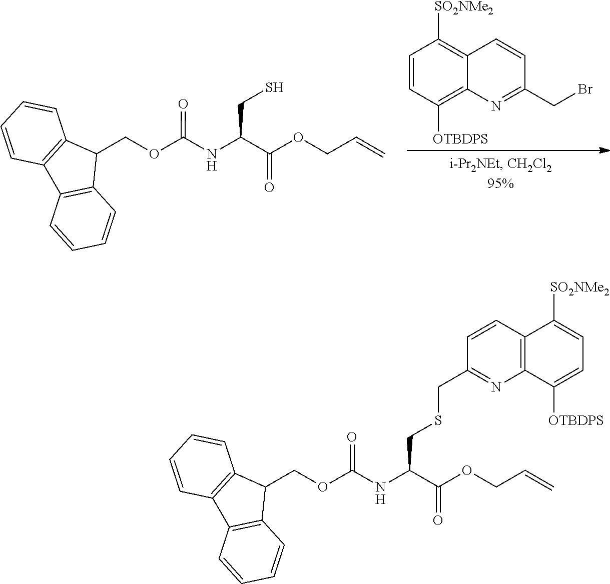

To an oven-dried, one-necked, 500 mL round-bottomed flask equipped with a stir bar and under positive N2 pressure was added Fmoc-Cys-OAllyl (4.98 mmol, 1.91 g, 1 equiv.) dissolved in anhydrous CH2Cl2 (70 mL). To this colorless solution was added Sox-Br₂ (4.98 mmol, 2.9 g, 1 equiv.) followed by freshly distilled DIEA (7.46 mmol, 1.3 mL, 1.5 equiv.). The reaction (pale yellow in color) was stirred at room temperature under the positive N2 pressure overnight and was monitored by TLC (Rf=0.36, 1:2 EtOAc in hexanes) until completion. The crude reaction was diluted with CH2Cl2 (400 mL), washed with sat'd. NH₄Cl (2×100 mL), H₂O (100 mL), brine (100 mL), dried (Na2SO4) and concentrated in vacuo. Flash column chromatography (SiO2; diameter: 70 mm; length: 26 cm; packing: 10% EtOAc in hexanes; load crude product in CH2Cl2; eluent: 10, 20, 30% EtOAc in hexanes) was used to isolate the product as a white solid. Yield: 95% (3.43 g).

1H NMR (400 MHz, CDCl₃, 20° C.) δ ppm: 1.18 (s, 9H), 2.82-2.67 (m, 2H), 2.72 (s, 6H), 3.57 (m, 2H), 4.24 (t, J=7.04 Hz, 1H), 4.45-4.34 (m, 2H), 4.68-4.48 (m, 3H), 5.22 (dd, J=10.42, 1.09 Hz, 1H), 5.28 (dd, J=17.19, 1.23 Hz, 1H), 5.51 (d, J=8.13 Hz, 1H), 5.89-5.78 (m, 1H), 7.11 (d, J=8.32 Hz, 1H), 7.34-7.29 (m, 6H), 7.45-7.35 (m, 4H), 7.53 (d, J=8.97 Hz, 1H), 7.61 (dd, J=7.21, 2.86 Hz, 2H), 7.78-7.75 (m, 6H), 7.99 (d, J=8.32 Hz, 1H), 8.95 (d, J=8.96 Hz, 1H), 13C NMR (100 MHz. CDCl₃, 20° C.) δ ppm: 20.0, 26.4, 34.1, 37.2, 38.2, 46.9, 53.3, 66.2, 67.1, 115.3, 118.9, 119.9, 122.3, 123.7, 125.0, 125.1, 127.0, 127.6, 127.6, 129.6, 131.1, 131.6, 133.3, 133.3, 134.2, 134.8, 140.3, 141.1, 143.6, 143.6, 155.5, 156.8, 157.1, 170.1.

HRMS (ESI): calcd for C49H51N3NaO7S2Si [M+Na]+: 908.2835, found: 908.2827.

c. Fmoc-Cys(Sox[TBDPS])-OH

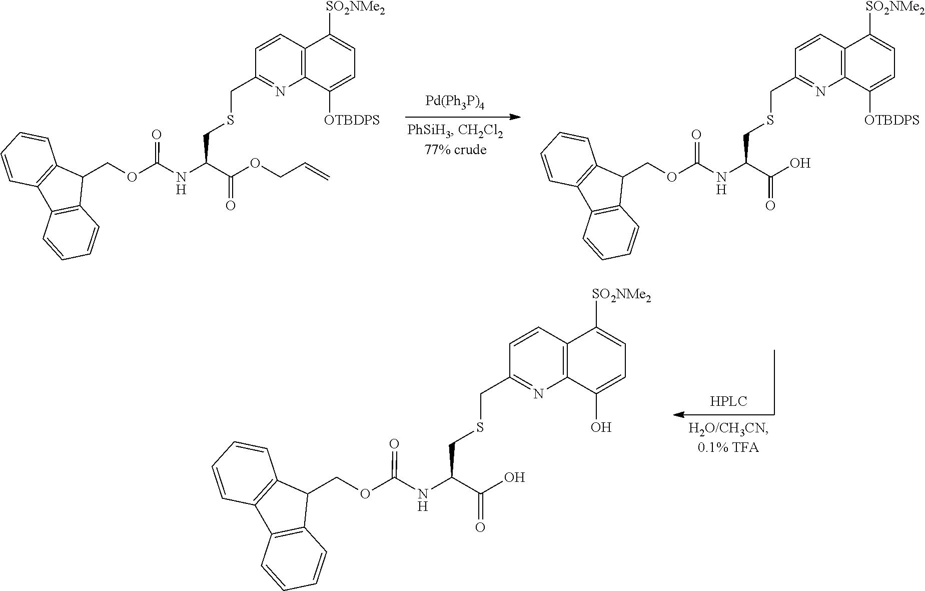

To an oven-dried, one-necked, 250 mL round-bottomed flask equipped with a stir bar and under positive N2 pressure was added Fmoc-Cys(Sox[TBPDS])-OAllyl (3.62 mmol, 3.21 g, 1 equiv.) dissolved in anhydrous and degassed CH2Cl2 (100 mL). To the pale red solution was added PhSiH3 (90.47 mmol, 11.2 mL, 25 equiv.) followed by Pd(PPh3)4 (0.145 mmol, 167.26 mg, 0.04 equiv.). After 10 min the reaction turned a deep red color. The resulting mixture was stirred at room temperature under the positive N2 pressure for 3 hours and was monitored by TLC (Rf=0.36, 10% MeOH in CH2Cl2). Upon completion of the reaction, the solvent was removed in vacuo. The crude mixture was passed through a short flash column (SiO2; diameter: 70 mm; length: 7 cm; packing: CH2Cl2; load crude product in CH2Cl2; eluent: 1, 2, 3, 4, 5, 10, 15% MeOH in CH2Cl2) to obtain 77%6 recovery of product. Analytical HPLC revealed one peak (tR=32.1 min; loading: dissolve 3 mg in DMSO; injection: 12 μL, method: 5% B (5 min)→20% B (1 min)→95% B (30 min), Abs: 280 nm and 316 nm) that was determined by ESI-MS to be Fmoc-C(Sox)-OH (calcd for C30H30N307S2 [M+H]+: 608.15, found: 608.1) since the acidic conditions of HPLC solvents remove the TBDPS protecting group. 1H NMR, prior to HPLC analysis, shows that the TBDPS protecting group is still present in the crude material. The amino acid was used in SPPS without further purification.

HRMS (ESI) of Fmoc-C(Sox[TBDPS])-OH: calcd for C46H47N3NaO7S2Si [M+Na]+: 868.2522, found: 868.2540.

### Example 20: Peptide Synthesis

a. Coupling Chemistry and Conditions

All peptides were synthesized using the standard Fmoc-based amino acid protection chemistry using a variety of methods. For example, in some cases, peptides were synthesized on Fmoc-PAL-PEG-PS resin (Applied Biosystems, 0.19 mmol/g) using either the on-resin alkylation (vide infra) or the Fmoc-C(Sox[TBDPS])-OH building block. In other cases, peptides were synthesized on Fmoc-Gly-NovaSyn TGT resin (Novabiochem, 0.20 mmol/g) using the C-Sox building block. The resin was swelled in CH2Cl2 (5 min.) and then DMF (5 min) prior to synthesis. All the amino acids except for Fmoc-C(Sox[TBDPS])-OH were coupled according to the following procedure: Fmoc deprotection (20% 4-methylpiperidine in DMF, 3×5 min), rinsing step (DMF, 5×), coupling step (amino acid/PyBOP/HOBt/DIEA, 6:6:6:6, 0.15 M in DMF, 30-45 min), rinsing step (DMF, 5 x; CH2Cl2, 5×). Fmoc-C(Sox[TBDPS])-OH was coupled in the following manner: amino acid/PyAOP/HOAt/DIEA, 2:2:2:5, 0.15 M in DMF, 2-12 hr. The coupling was repeated if necessary (amino acid/PyAOP/HOAt/DIEA, 1:1:1:3, 0.15 M in DMF, 2-12 hr) as determined by the TNBS test for free amines. It is important to wash the resin rigorously (DMF followed by CH2Cl2) to remove excess amino acid before performing any tests for free amines. This is particularly necessary after coupling of Fmoc-C(Sox[TBDPS])-OH due to its deep red color, which does not affect its coupling efficiency. At the end of the synthesis, the Fmoc group was removed with 20% 4-methylpiperidine in DMF (3×5 min.) and the resin was rinsed with DMF (5×). The resin-attached free amines were capped by exposure to Ac2O (20 equiv.) and pyridine (20 equiv.) in DMF for 30 min. The resin was rinsed with DMF (5×), CH2Cl2 (5×) and subjected to 20% 4-methylpiperidine in DMF (3×5 min.) to remove any Sox aryl esters that might have formed during acetylation. The resin was finally washed with DMF, CH2Cl2, MeOH (5× each) and dried under vacuum. Different variations on the above methods may be required to improve yield and purity of the desired chemosensor.

b. On-Resin Alkylation of Peptides with Sox-Br

Resin-bound peptides (50 mg, 0.0095 mmol, 1 equiv.) incorporating Cys(Mmt) were swelled in CH2Cl2, then DMF (5 min each). The Mmt protecting group was removed from the resin-bound peptide by bubbling N2 through a solution of 1% TFA, 5% TIS in CH₂Cl₂ (4×20 min or until most of the yellow color due to the Mmt cation has disappeared). The resin then was subjected to rigorous washing with CH2Cl2 (5×) and DMF (5×). Anhydrous DMF (200 μL) was added to the resin followed by freshly distilled tetramethylguanidine (5.96 μL, 0.0475 mmol, 5 equiv.). The mixture was incubated for 2-3 min. Sox-Br (17 mg, 0.0285 mmol, 3 equiv.) was dissolved in anhydrous DMF (150 μL) and added to the resin. After ca. 12 hours of reaction time, the excess reagents were drained and the resin washed with DMF, CH₂C₂, MeOH, CH₂Cl₂ (5× each). Different variations on the above methods may be required to improve yield and purity of the desired chemosensor.

c. Side Chain Deprotection and Cleavage from Resin

The resin cleavage and protecting group removal was achieved by exposing the resin-bound peptides to TFA/EDT/H2O/TIS (94:2.5:2.5:1% v/v) for sequences containing easily oxidized residues (e.g. Cys, Met, Trp) or TFA/H₂O/TIS (95:2.5:2.5% v/v) for sequences without such residues (C-Sox does not require EDT in the cleavage cocktail). The resulting solution was concentrated under a stream of N2 and precipitated by addition of cold Et2O. The pellet was triturated with cold Et2O (3×), redissolved in water, filtered and lyophilized. The peptides were purified by preparative reverse-phase HPLC using UV detection at either 228 nm (amide bond absorption) and 280 nm (Fmoc, Trp, and/or Tyr absorption) or 228 nm and 316 nm (Sox absorption). Only fractions showing a single peak of correct mass by analytical HPLC were used in further experiments.

Characterization of Purified Peptides.

The purity of the synthetic peptides was assessed by reversed-phase HPLC and identity was confirmed by ESI-MS, prior to characterizing specificity (testing with a panel of protein kinases) and enzyme kinetic properties with the target kinase.

### Example 21: Representative Peptides

The following peptides were synthesized as previous described, and characterized as followed:

Ac-EEPITI-C(Sx)-VP-amide (AQT0026-Sx): MW: calc. (mono MW): 1366.6 Da; exp (avg): 1367.6 Da; HPLC-purity: expected 95+%; measured: 99.85%; Mass Spec. TOF: 684.3; Tandem Mass Spec.: 114.0, 171.0, 227.9; 510.2; 580.2; 673.3; 682.5; 786.4; 952.4; 1066.5; 1195.6; 1252.5.

Ac-EDPEYI-C(Sx)-IP-amide (AQT0099-BBCSx): MW: calc. (mono MW): 1382.5 Da; exp (avg): 1383.5 Da; HPLC-purity: expected 95+%; measured: 95.51%; Mass Spec. TOF: 892.2; Tandem Mass Spec.; 114.1, 69.1, 286.2, 548.9, 694.4, 805.9.

Ac-GRPRAATF-C(SY)-EG-amide (AQT0076-Sx): MW: calc. (mono MW): 1468.6 Da; exp (avg): 1469.5 Da; HPLC-purity: expected 95+%; measured: 96.35%; Mass Spec. TOF: 735.3; Tandem Mass Spec.: 119.0, 227.1, 268.9, 285.1, 296.0, 633.3, 733.8, 870.4, 967.4, 1375.5.

Ac-C(S)-LSPGPF-amide (AQT0082-Sx): MW: calc. (mono MW): 1024.4 Da; exp (avg): 1025.2 Da; HPLC-purity: expected 95+%; measured: 99.28%; Mass Spec. TOF: 1025.41; Tandem Mass Spec.: 399.2, 415.2, 502.2, 615.3, 1007.4, 1024.4.

While the invention has been described with reference to certain embodiments, other features may be included without departing from the spirit and scope of the invention. It is therefore intended that the foregoing detailed description be regarded as illustrative rather than limiting, and that it be understood that it is the following claims, including all equivalents, that are intended to define the spirit and scope of this invention

1.-112. (canceled)

113. A method for detecting kinase activity, comprising the steps of:
a) providing a compound;
b) contacting the compound with a sample comprising Mg²⁺, a phosphate source, and a kinase; and
c) analyzing for the presence of a phosphorylated peptide product;
wherein:
the compound is an oligopeptide comprising a fluorophore; and a S, T or Y residue at the +2 or −2 position relative to the fluorophore.

114.-117. (canceled)

118. A method, comprising the step of:
determining phosphorylation of a compound using fluorescence of the compound; wherein:
the compound is an oligopeptide comprising a fluorophore; and a (phospho)S, a (phospho)T, or a (phospho)Y residue at the +2 or −2 position relative to the fluorophore.

119.-120. (canceled)

121. A compound selected from the group consisting of: X-EEPIY*V-C(Sx)-FG-Z (SEQ ID NO: 531), X-EEPIY*V-C(Sx)-VK-Z (SEQ ID NO: 547), X-EEPEY*I-C(Sx)-FG-Z (SEQ ID NO: 407), X-EEPEY*I-C(Sx)-FK-Z (SEQ ID NO: 408), X-EEPEY*I-C(Sx)-FP-Z (SEQ ID NO: 409), X-EEPEY*I-C(Sx)-IG-Z (SEQ ID NO: 410), X-EEPEY*I-C(Sx)-IK-Z (SEQ ID NO: 411), X-EEPEY*I-C(Sx)-IP-Z (SEQ ID NO: 412), X-EEPEY*I-C(Sx)-MG-Z (SEQ ID NO: 413), X-EEPEY*I-C(Sx)-MK-Z (SEQ ID NO: 414), X-EEPEY*I-C(Sx)-MP-Z (SEQ ID NO: 415), X-EEPEY*I-C(Sx)-VG-Z (SEQ ID NO: 416), X-EEPEY*I-C(Sx)-VK-Z (SEQ ID NO: 417), X-EEPEY*V-C(Sx)-FG-Z (SEQ ID NO: 419), X-EEPEY*V-C(Sx)-FK-Z (SEQ ID NO: 420), X-EEPEY*V-C(Sx)-FP-Z (SEQ ID NO: 421), X-EEPEY*V-C(Sx)-IG-Z (SEQ ID NO: 422), X-EEPEY*V-C(Sx)-IK-Z (SEQ ID NO: 423), X-EEPEY*V-C(Sx)-IP-Z (SEQ ID NO: 424), X-EEPEY*V-C(Sx)-MG-Z (SEQ ID NO: 425), X-EEPEY*V-C(Sx)-MK-Z (SEQ ID NO: 426), X-EEPEY*V-C(Sx)-MP-Z (SEQ ID NO: 427), X-EEPEY*V-C(Sx)-VG-Z (SEQ ID NO: 428), X-EEPEY*V-C(Sx)-VK-Z (SEQ ID NO: 429), and X-EEPEY*V-C(Sx)-VP-Z (SEQ ID NO: 430), X-EEPIY*F-C(Sx)-FG-Z (SEQ ID NO: 458), X-EEPIY*F-C(Sx)-FK-Z (SEQ ID NO: 459), X-EEPIY*F-C(Sx)-FP-Z (SEQ ID NO: 461), X-EEPIY*F-C(Sx)-IG-Z (SEQ ID NO: 462), X-EEPIY*F-C(Sx)-IK-Z (SEQ ID NO: 463), X-EEPIY*F-C(Sx)-IP-Z (SEQ ID NO: 465), X-EEPIY*F-C(Sx)-MG-Z (SEQ ID NO: 469), X-EEPIY*F-C(Sx)-MK-Z (SEQ ID NO: 470), X-EEPIY*F-C(Sx)-MP-Z (SEQ ID NO: 472), X-EEPIY*F-C(Sx)-VG-Z (SEQ ID NO: 473), X-EEPIY*F-C(Sx)-VK-Z (SEQ ID NO: 474), X-EEPIY*I-C(Sx)-FG-Z (SEQ ID NO: 483), X-EEPIY*I-C(Sx)-FK-Z (SEQ ID NO: 484), X-EEPIY*I-C(Sx)-IG-Z (SEQ ID NO: 487), X-EEPIY*I-C(Sx)-IK-Z (SEQ ID NO: 488), X-EEPIY*I-C(Sx)-MG-Z (SEQ ID NO: 494), X-EEPIY*I-C(Sx)-MK-Z (SEQ ID NO: 495), X-EEPIY*V-C(Sx)-FK-Z (SEQ ID NO: 532), X-EEPIY*V-C(Sx)-FP-Z (SEQ ID NO: 534), X-EEPIY*V-C(Sx)-IG-Z (SEQ ID NO: 535), X-EEPIY*V-C(Sx)-IK-Z (SEQ ID NO: 536), X-EEPIY*V-C(Sx)-IP-Z (SEQ ID NO: 538), X-EEPIY*V-C(Sx)-MG-Z (SEQ ID NO: 542), X-EEPIY*V-C(Sx)-MK-Z (SEQ ID NO: 543), X-EEPIY*V-C(Sx)-MP-Z (SEQ ID NO: 545), X-EEPIY*V-C(Sx)-VG-Z (SEQ ID NO: 546), X-EEPPD-C(Sx)-QY*[pY]NDFPGK-Z (SEQ ID NO: 974), X-EEPPD-C(Sx)-QY*YNDFPGK-Z (SEQ ID NO: 973), X-EGRNPGFY*V-C(Sx)-ANPMP-Z (SEQ ID NO: 967), X-EGSFESRY*Q-C(Sx)-PFEDF-Z (SEQ ID NO: 965), X-EPIQEANY*V-C(Sx)-MTPGT-Z (SEQ ID NO: 955), X-EPPDHQYY*N-C(Sx)-FPGKE-Z (SEQ ID NO: 975), X-EQEDEPE-C(Sx)-DY*EEVLE-Z (SEQ ID NO: 5003), X-EQEDEPE-C(Sx)-DY*FEWLE-Z (SEQ ID NO: 5004), X-EQEDEPEGDY*E-C(Sx)-VLE-Z (SEQ ID NO: 5005), X-EQEDEPEGDY*F-C(Sx)-WLE-Z (SEQ ID NO: 5006), X-EVSETDDY*A-C(Sx)-DEE-Z (SEQ ID NO: 5007), X-FAGKE-C(Sx)-TY*[pY]QGRHLG-Z (SEQ ID NO: 980), X-FAGKE-C(Sx)-TY*YQGRHLG-Z (SEQ ID NO: 979), X-FD[Nle]DR-C(Sx)-IY*RMSFFRK-Z (SEQ ID NO: 5008), X-FD[Nle]DRDIY*R-C(Sx)-SFFRK-Z (SEQ ID NO: 5009), X-FDKDGNGY*I-C(Sx)-AAELR-Z (SEQ ID NO: 930), X-FDMDR-C(Sx)-IY*RMSFFRK-Z (SEQ ID NO: 5010), X-FDMDRDIY*R-C(Sx)-SFFRK-Z (SEQ ID NO: 5011), X-FEEEEY*I-C(Sx)-Z (SEQ ID NO: 5012), X-FEEEY*I-C(Sx)-IV-Z (SEQ ID NO: 5013), X-FTDRL-C(Sx)-QY*ISTRGLG-Z (SEQ ID NO: 919), X-FTDRLQQY*I-C(Sx)-TRGLG-Z (SEQ ID NO: 920), and X-FVSETDDY*A-C(Sx)-IIDEE-Z (SEQ ID NO: 5014); or
a compound selected from the group consisting of: X-C(Sx)-HS*LPRFNR-Z (SEQ ID NO: 1409), X-C(Sx)-FS*LRRKAA-Z (SEQ ID NO: 1361), X-C(Sx)-FS*LRRKAD-Z (SEQ ID NO: 1362), X-C(Sx)-FS*LRRKAE-Z (SEQ ID NO: 1363), X-C(Sx)-FS*LRRKAF-Z (SEQ ID NO: 1364), X-C(Sx)-FS*LRRKAG-Z (SEQ ID NO: 1365), X-C(Sx)-FS*LRRKAH-Z (SEQ ID NO: 1366), X-C(Sx)-FS*LRRKAI-Z (SEQ ID NO: 1367), X-C(Sx)-FS*LRRKAL-Z (SEQ ID NO: 1368), X-C(Sx)-FS*LRRKAN-Z (SEQ ID NO: 1369), X-C(Sx)-FS*LRRKAP-Z (SEQ ID NO: 1370), X-C(Sx)-FS*LRRKAQ-Z (SEQ ID NO: 1371), X-C(Sx)-FS*LRRKAR-Z (SEQ ID NO: 1372), X-C(Sx)-FS*LRRKAV-Z (SEQ ID NO: 1373), X-C(Sx)-FS*LRRKAW-Z (SEQ ID NO: 1374), X-C(Sx)-FS*LRRKDK-Z (SEQ ID NO: 1375), X-C(Sx)-FS*LRRKEK-Z (SEQ ID NO: 1376), X-C(Sx)-FS*LRRKFK-Z (SEQ ID NO: 1377), X-C(Sx)-FS*LRRKGK-Z (SEQ ID NO: 1378), X-C(Sx)-FS*LRRKHK-Z (SEQ ID NO: 1379), X-C(Sx)-FS*LRRKIK-Z (SEQ ID NO: 1380), X-C(Sx)-FS*LRRKKK-Z (SEQ ID NO: 1381), X-C(Sx)-FS*LRRKLK-Z (SEQ ID NO: 1382), X-C(Sx)-FS*LRRKNK-Z (SEQ ID NO: 1383), X-C(Sx)-FS*LRRKPK-Z (SEQ ID NO: 1384), X-C(Sx)-FS*LRRKQK-Z (SEQ ID NO: 1385), X-C(Sx)-FS*LRRKRK-Z (SEQ ID NO: 1386), X-C(Sx)-FS*LRRKVK-Z (SEQ ID NO: 1387), X-C(Sx)-FS*LRRKWK-Z (SEQ ID NO: 1388), X-C(Sx)-FS*LRRLAK-Z (SEQ ID NO: 1389), X-C(Sx)-FS*LRRNAK-Z (SEQ ID NO: 1390), X-C(Sx)-FS*LRRPAK-Z (SEQ ID NO: 1391), X-C(Sx)-FS*LRRQAK-Z (SEQ ID NO: 1392), X-C(Sx)-FS*LRRRAK-Z (SEQ ID NO: 1393), X-C(Sx)-FS*LRRVAK-Z (SEQ ID NO: 1394), X-C(Sx)-FS*LRRWAK-Z (SEQ ID NO: 1395), X-C(Sx)-FS*LRVKAK-Z (SEQ ID NO: 1396), X-C(Sx)-FS*LRWKAK-Z (SEQ ID NO: 1397), X-C(Sx)-FS*LVRKAK-Z (SEQ ID NO: 1398), X-C(Sx)-FS*LWRKAK-Z (SEQ ID NO: 1399), X-C(Sx)-FS*NRRKAK-Z (SEQ ID NO: 1400), X-C(Sx)-FS*PRRKAK-Z (SEQ ID NO: 1401), X-C(Sx)-FS*QRRKAK-Z (SEQ ID NO: 1402), X-C(Sx)-FS*RRRKAK-Z (SEQ ID NO: 1403), X-C(Sx)-FS*VRRKAK-Z (SEQ ID NO: 1404), X-C(Sx)-FS*WRRKAK-Z (SEQ ID NO: 1405), X-C(Sx)-GS*LRRKAK-Z (SEQ ID NO: 1406), X-C(Sx)-HS*LKRGNR-Z (SEQ ID NO: 1407), X-C(Sx)-HS*LKRKNR-Z (SEQ ID NO: 1408), X-C(Sx)-HS*LPRKNK-Z (SEQ ID NO: 1410), X-C(Sx)-HS*LRRKAK-Z (SEQ ID NO: 1411), X-C(Sx)-IS*LRRKAK-Z (SEQ ID NO: 1412), X-C(Sx)-KS*LRRKAK-Z (SEQ ID NO: 1413), X-C(Sx)-KS*PAKEKAKSPEK-Z (SEQ ID NO: 1414), X-C(Sx)-LS*FAEG-Z (SEQ ID NO: 1415), X-C(Sx)-LS*FAEPG-Z (SEQ ID NO: 1416), X-C(Sx)-LS*FRRR-Z (SEQ ID NO: 1019), X-C(Sx)-LS*LRRKAK-Z (SEQ ID NO: 1417), X-C(Sx)-LS*QEAFADLWKK-Z (SEQ ID NO: 1418), X-C(Sx)-NS*LRRKAK-Z (SEQ ID NO: 1419), X-C(Sx)-PS*LRRKAK-Z (SEQ ID NO: 1420), X-C(Sx)-QS*LRRKAK-Z (SEQ ID NO: 1421), X-C(Sx)-QS*QEAFADLWKK-Z (SEQ ID NO: 1422), X-C(Sx)-QS*QETFS*DLWKK-Z (SEQ ID NO: 1423), and X-C(Sx)-RS*LRRKAK-Z (SEQ ID NO: 1424); or
a compound selected from the group consisting of: X-EDPEY*I-C(Sx)-IP-Z (SEQ ID NO: 304), X-DVVDADEY*L-C(Sx)-PQQGF-Z (SEQ ID NO: 938), X-ED-C(Sx)-DY*EWPSA-Z (SEQ ID NO: 4867), X-EDE-C(Sx)-IY*EELDEP-Z (SEQ ID NO: 4868), X-EDEDIY*E-C(Sx)-LDEP-Z (SEQ ID NO: 4869), X-EDEG-C(Sx)-RY*LKLEED-Z (SEQ ID NO: 4870), X-EDEGDRY*L-C(Sx)-LEED-Z (SEQ ID NO: 4871), X-EDGGDDIY*E-C(Sx)-IIKVE-Z (SEQ ID NO: 986), X-EDPDY*E-C(Sx)-PSA-Z (SEQ ID NO: 4872), X-EDPDY*F-C(Sx)-FG-Z (SEQ ID NO: 251), X-EDPDY*F-C(Sx)-FK-Z (SEQ ID NO: 252), X-EDPDY*F-C(Sx)-FP-Z (SEQ ID NO: 253), X-EDPDY*F-C(Sx)-IG-Z (SEQ ID NO: 254), X-EDPDY*F-C(Sx)-IK-Z (SEQ ID NO: 255), X-EDPDY*F-C(Sx)-IP-Z (SEQ ID NO: 256), X-EDPDY*F-C(Sx)-MG-Z (SEQ ID NO: 257), X-EDPDY*F-C(Sx)-MK-Z (SEQ ID NO: 258), X-EDPDY*F-C(Sx)-MP-Z (SEQ ID NO: 259), X-EDPDY*F-C(Sx)-VG-Z (SEQ ID NO: 260), X-EDPDY*F-C(Sx)-VK-Z (SEQ ID NO: 261), X-EDPDY*F-C(Sx)-VP-Z (SEQ ID NO: 262), X-EDPDY*I-C(Sx)-FG-Z (SEQ ID NO: 263), X-EDPDY*I-C(Sx)-FK-Z (SEQ ID NO: 264), X-EDPDY*I-C(Sx)-FP-Z (SEQ ID NO: 265), X-EDPDY*I-C(Sx)-IG-Z (SEQ ID NO: 266), X-EDPDY*I-C(Sx)-IK-Z (SEQ ID NO: 267), X-EDPDY*I-C(Sx)-IP-Z (SEQ ID NO: 268), X-EDPDY*I-C(Sx)-MG-Z (SEQ ID NO: 269), X-EDPDY*I-C(Sx)-MK-Z (SEQ ID NO: 270), X-EDPDY*I-C(Sx)-MP-Z (SEQ ID NO: 271), X-EDPDY*I-C(Sx)-VG-Z (SEQ ID NO: 272), X-EDPDY*I-C(Sx)-VK-Z (SEQ ID NO: 273), X-EDPDY*I-C(Sx)-VP-Z (SEQ ID NO: 274), X-EDPDY*V-C(Sx)-FG-Z (SEQ ID NO: 275), X-EDPDY*V-C(Sx)-FK-Z (SEQ ID NO: 276), X-EDPDY*V-C(Sx)-FP-Z (SEQ ID NO: 277), X-EDPDY*V-C(Sx)-IG-Z (SEQ ID NO: 278), X-EDPDY*V-C(Sx)-IK-Z (SEQ ID NO: 279), X-EDPDY*V-C(Sx)-IP-Z (SEQ ID NO: 280), X-EDPDY*V-C(Sx)-MG-Z (SEQ ID NO: 281), X-EDPDY*V-C(Sx)-MK-Z (SEQ ID NO: 282), X-EDPDY*V-C(Sx)-MP-Z (SEQ ID NO: 283), X-EDPDY*V-C(Sx)-VG-Z (SEQ ID NO: 284), X-EDPDY*V-C(Sx)-VK-Z (SEQ ID NO: 285), X-EDPDY*V-C(Sx)-VP-Z (SEQ ID NO: 286), X-EDPEY*F-C(Sx)-FG-Z (SEQ ID NO: 287), X-EDPEY*F-C(Sx)-FK-Z (SEQ ID NO: 288), X-EDPEY*F-C(Sx)-FP-Z (SEQ ID NO: 289), X-EDPEY*F-C(Sx)-IG-Z (SEQ ID NO: 290), X-EDPEY*F-C(Sx)-IK-Z (SEQ ID NO: 291), X-EDPEY*F-C(Sx)-IP-Z (SEQ ID NO: 292), X-EDPEY*F-C(Sx)-MG-Z (SEQ ID NO: 293), X-EDPEY*F-C(Sx)-MK-Z (SEQ ID NO: 294), X-EDPEY*F-C(Sx)-MP-Z (SEQ ID NO: 295), X-EDPEY*F-C(Sx)-VG-Z (SEQ ID NO: 296), X-EDPEY*F-C(Sx)-VK-Z (SEQ ID NO: 297), X-EDPEY*F-C(Sx)-VP-Z (SEQ ID NO: 298), X-EDPEY*I-C(Sx)-FG-Z (SEQ ID NO: 299), X-EDPEY*I-C(Sx)-FK-Z (SEQ ID NO: 300), X-EDPEY*I-C(Sx)-FP-Z (SEQ ID NO: 301), X-EDPEY*I-C(Sx)-IG-Z (SEQ ID NO: 302), X-EDPEY*I-C(Sx)-IK-Z (SEQ ID NO: 303), X-EDPEY*I-C(Sx)-MG-Z (SEQ ID NO: 305), X-EDPEY*I-C(Sx)-MK-Z (SEQ ID NO: 306), X-EDPEY*I-C(Sx)-MP-Z (SEQ ID NO: 307), X-EDPEY*I-C(Sx)-VG-Z (SEQ ID NO: 308), X-EDPEY*I-C(Sx)-VK-Z (SEQ ID NO: 309), X-EDPEY*I-C(Sx)-VP-Z (SEQ ID NO: 310), X-EDPEY*V-C(Sx)-FG-Z (SEQ ID NO: 311), X-EDPEY*V-C(Sx)-FK-Z (SEQ ID NO: 312), X-EDPEY*V-C(Sx)-FP-Z (SEQ ID NO: 313), X-EDPEY*V-C(Sx)-IG-Z (SEQ ID NO: 314), X-EDPEY*V-C(Sx)-IK-Z (SEQ ID NO: 315), X-EDPEY*V-C(Sx)-IP-Z (SEQ ID NO: 316), X-EDPEY*V-C(Sx)-MG-Z (SEQ ID NO: 317), X-EDPEY*V-C(Sx)-MK-Z (SEQ ID NO: 318), X-EDPEY*V-C(Sx)-MP-Z (SEQ ID NO: 319), X-EDPEY*V-C(Sx)-VG-Z (SEQ ID NO: 320), X-EDPEY*V-C(Sx)-VK-Z (SEQ ID NO: 321), and X-EDPEY*V-C(Sx)-VP-Z (SEQ ID NO: 322); or
a compound selected from the group consisting of: X-KKKKVKKRPQRADS*D-C(Sx)-FA-Z (SEQ ID NO: 3457), X-KKKKRKKRPQRATSD-C(Sx)-FS*-Z (SEQ ID NO: 3429), X-KKKKRKKRPQRATSN-C(Sx)-FS*-Z (SEQ ID NO: 3430), X-KKKKTKKRPQRADS*D-C(Sx)-FA-Z (SEQ ID NO: 3431), X-KKKKTKKRPQRADS*D-C(Sx)-FS-Z (SEQ ID NO: 3432), X-KKKKTKKRPQRADS*N-C(Sx)-FA-Z (SEQ ID NO: 3433), X-KKKKTKKRPQRADS*N-C(Sx)-FS-Z (SEQ ID NO: 3434), X-KKKKTKKRPQRADSD-C(Sx)-FS*-Z (SEQ ID NO: 3435), X-KKKKTKKRPQRADSN-C(Sx)-FS*-Z (SEQ ID NO: 3436), X-KKKKTKKRPQRAES*D-C(Sx)-FA-Z (SEQ ID NO: 3437), X-KKKKTKKRPQRAES*D-C(Sx)-FS-Z (SEQ ID NO: 3438), X-KKKKTKKRPQRAES*N-C(Sx)-FA-Z (SEQ ID NO: 3439), X-KKKKTKKRPQRAES*N-C(Sx)-FS-Z (SEQ ID NO: 3440), X-KKKKTKKRPQRAESD-C(Sx)-FS*-Z (SEQ ID NO: 3441), X-KKKKTKKRPQRAESN-C(Sx)-FS*-Z (SEQ ID NO: 3442), X-KKKKTKKRPQRAHS*D-C(Sx)-FA-Z (SEQ ID NO: 3443), X-KKKKTKKRPQRAHS*D-C(Sx)-FS-Z (SEQ ID NO: 3444), X-KKKKTKKRPQRAHS*N-C(Sx)-FA-Z (SEQ ID NO: 3445), X-KKKKTKKRPQRAHS*N-C(Sx)-FS-Z (SEQ ID NO: 3446), X-KKKKTKKRPQRAHSD-C(Sx)-FS*-Z (SEQ ID NO: 3447), X-KKKKTKKRPQRAHSN-C(Sx)-FS*-Z (SEQ ID NO: 3448), X-KKKKTKKRPQRATS*D-C(Sx)-FA-Z (SEQ ID NO: 3449), X-KKKKTKKRPQRATS*D-C(Sx)-FS-Z (SEQ ID NO: 3450), X-KKKKTKKRPQRATS*N-C(Sx)-FA-Z (SEQ ID NO: 3451), X-KKKKTKKRPQRATS*N-C(Sx)-FS-Z (SEQ ID NO: 3452), X-KKKKTKKRPQRATSD-C(Sx)-FS*-Z (SEQ ID NO: 3453), X-KKKKTKKRPQRATSN-C(Sx)-FS*-Z (SEQ ID NO: 3454), X-KKKKVKKRPQRA[hE]S*D-C(Sx)-FS-Z (SEQ ID NO: 3455), X-KKKKVKKRPQRA[hE]SD-C(Sx)-FS*-Z (SEQ ID NO: 3456), X-KKKKVKKRPQRADS*D-C(Sx)-FS-Z (SEQ ID NO: 3458), X-KKKKVKKRPQRADS*N-C(Sx)-FA-Z (SEQ ID NO: 3459), X-KKKKVKKRPQRADS*N-C(Sx)-FS-Z (SEQ ID NO: 3460), X-KKKKVKKRPQRADSD-C(Sx)-FS*-Z (SEQ ID NO: 3461), and X-KKKKVKKRPQRADSN-C(Sx)-FS*-Z (SEQ ID NO: 3462); or
a compound selected from the group consisting of: X-EEPIY*I-C(Sx)-VP-Z (SEQ ID NO: 30), X-GAEEPIY*I-C(Sx)-VPAKKKG-Z (SEQ ID NO: 42), X-EEEIY*F-C(Sx)-dP-(2-Nal)-G-Z (SEQ ID NO: 16), X-EEEIY*F-C(Sx)-Z (SEQ ID NO: 17), X-EEIY*F-C(Sx)-Z (SEQ ID NO: 18), X-EIY*F-C(Sx)-Z (SEQ ID NO: 19), X-IY*F-C(Sx)-Z (SEQ ID NO: 20), X-Y*F-C(Sx)-Z (SEQ ID NO: 21), X-EEEEY*I-C(Sx)-dP-(2-Nal)-G-Z (SEQ ID NO: 22), X-EEEEY*I-C(Sx)-VG-Z (SEQ ID NO: 23), X-DPQEY*I-C(Sx)-LP-Z (SEQ ID NO: 24), X-EDEDY*E-C(Sx)-VG-Z (SEQ ID NO: 25), X-EAEAIY*A-C(Sx)-dP-(2-Nal)-G-Z (SEQ ID NO: 26), X-EAEAIY*A-C(Sx)-PF-Z (SEQ ID NO: 27), X-EAEAIY*A-C(Sx)-P-Z (SEQ ID NO: 28), X-EAEAIY*A-C(Sx)-Z (SEQ ID NO: 29), X-DEQIY*W-C(Sx)-IA-Z (SEQ ID NO: 31), X-KKGEAIY*A-C(Sx)-dP-(2-Nal)-G-Z (SEQ ID NO: 32), X-KKGEAIY*A-C(Sx)-PFAKKK-Z (SEQ ID NO: 33), X-KKGE-C(Sx)-IY*AAPFAKKK-Z (SEQ ID NO: 34), X-E-C(Sx)-IY*AAPF-Z (SEQ ID NO: 35), X-E-C(Sx)-IY*A-Z (SEQ ID NO: 36), X-E-C(Sx)-IY*-Z (SEQ ID NO: 37), X-C(Sx)-IY*-Z (SEQ ID NO: 38), X-KKGEAIY*A-C(Sx)-PF-Z (SEQ ID NO: 39), X-PE-C(Sx)-IY*ATPG-Z (SEQ ID NO: 40), and X-PEVIY*A-C(Sx)-PG-Z (SEQ ID NO: 41); or
a compound selected from the group consisting of: X-C(Sx)-KT*PKKAKKL-Z (SEQ ID NO: 212), X-C(Sx)-QS*PKKG-Z (SEQ ID NO: 207), X-KKKVLTQMGSPSIR-C(Sx)-SS*VS-Z (SEQ ID NO: 139), X-KKKVLTQMGSPSI-C(Sx)-SS*[pS]VS-Z (SEQ ID NO: 140), X-SGRSRRKS*P-C(Sx)-PSPHP-Z (SEQ ID NO: 141), X-SGRSR-C(Sx)-KS*PRPSPHP-Z (SEQ ID NO: 142), X-SGRSR-C(Sx)-KS*VRRSPHP-Z (SEQ ID NO: 143), X-KKLNRTLS*F-C(Sx)-EPG-Z (SEQ ID NO: 144), X-KKRPQRATS*N-C(Sx)-FAM-Z (SEQ ID NO: 145), X-RGRSRRDS*F-C(Sx)-VSPHY-Z (SEQ ID NO: 146), X-RKRSRRKS*F-C(Sx)-VLSSL-Z (SEQ ID NO: 147), X-AKRRRLSSLRASTSK-C(Sx)-ES*SQK-Z (SEQ ID NO: 148), X-AKRRRLSSLRASTSKSES*S-C(Sx)-K-Z (SEQ ID NO: 149), X-KMQPPR-C(Sx)-RS*SIMSIT-Z (SEQ ID NO: 150), X-KMQPPRSRS*S-C(Sx)-MSIT-Z (SEQ ID NO: 151), X-SGRSRPLS*P-C(Sx)-PSPHY-Z (SEQ ID NO: 152), X-LIDS*M-C(Sx)-NSFVG-Z (SEQ ID NO: 153), X-LIDSM-C(Sx)-NS*FVG-Z (SEQ ID NO: 154), X-PKRSR-C(Sx)-SS*YPNGHRD-Z (SEQ ID NO: 155), X-PKRSRSSS*V-C(Sx)-PSHSY-Z (SEQ ID NO: 156), X-SGLEDDDY*V-C(Sx)-PGGHW-Z (SEQ ID NO: 157), X-ALKLVRYPS*F-C(Sx)-ITAKKK-Z (SEQ ID NO: 158), X-AMRLERQDS*I-C(Sx)-YPKKK-Z (SEQ ID NO: 159), X-KKKVSRSGLYRSPS*M-C(Sx)-ENLNRPR-Z (SEQ ID NO: 160), X-KKVSRSGLYRSPS*M-C(Sx)-E-Z (SEQ ID NO: 161), X-KKVSRSGLYR-C(Sx)-PS*MPE-Z (SEQ ID NO: 162), X-GRPRTSS*F-C(Sx)-EPG-Z (SEQ ID NO: 163), X-LRREILS*R-C(Sx)-PSYRK-Z (SEQ ID NO: 164), X-LRREILSR-C(Sx)-PS*YRK-Z (SEQ ID NO: 165), X-LRRE-C(Sx)-LS*RRPSYRK-Z (SEQ ID NO: 166), X-LRREILSRRPS*Y-C(Sx)-K-Z (SEQ ID NO: 167), X-FQREGFGRQS*M-C(Sx)-EKR-Z (SEQ ID NO: 168), X-FQREGFG-C(Sx)-QS*MSEKR-Z (SEQ ID NO: 169), X-RS*R-C(Sx)-RSRSRSRSRSR-Z (SEQ ID NO: 170), X-RSR-C(Sx)-RS*RSRSRSRSR-Z (SEQ ID NO: 171), X-RSRSRS*R-C(Sx)-RSRSRSR-Z (SEQ ID NO: 172), X-RSRSRSR-C(Sx)-RS*RSRSR-Z (SEQ ID NO: 173), X-RSRSRSRSRS*R-C(Sx)-RSR-Z (SEQ ID NO: 174), X-RSRSRSRSRSR-C(Sx)-RS*R-Z (SEQ ID NO: 175), X-ILLSELS*R-C(Sx)-RIR-Z (SEQ ID NO: 176), X-ILLSESS*R-C(Sx)-RIR-Z (SEQ ID NO: 177), X-ILLR-C(Sx)-SS*RRIRR-Z (SEQ ID NO: 178), X-ILLS-C(Sx)-SS*RRRIRSGNKLGRIGRN-Z (SEQ ID NO: 179), X-ILLS-C(Sx)-SS*RRRIRSGNKLGRIGRN-Z (SEQ ID NO: 179), X-GKRGD-C(Sx)-KS*VSRSSSS-Z (SEQ ID NO: 180), X-SGRSR-C(Sx)-DS*PRPGTHK-Z (SEQ ID NO: 181), X-SKRSR-C(Sx)-KS*PRRSLSY-Z (SEQ ID NO: 182), X-R-C(Sx)-LS*FRRR-Z (SEQ ID NO: 183), X-PLSRT*L-C(Sx)-VAAKK-Z (SEQ ID NO: 184), X-KKKKK-C(Sx)-FS*FKKSFKLSGFSFKKNKK-Z (SEQ ID NO: 185), X-KKKKKRFS*F-C(Sx)-KSFKLSGFSFKKNKK-Z (SEQ ID NO: 186), X-KKKKKRFSF-C(Sx)-KS*FKLSGFSFKKNKK-Z (SEQ ID NO: 187), X-KKKKKRFSFKKS*F-C(Sx)-LSGFSFKKNKK-Z (SEQ ID NO: 188), X-KKKKKRFSFKKSF-C(Sx)-LS*GFSFKKNKK-Z (SEQ ID NO: 189), X-KKKKKRFSFKKSFKLSGFS*F-C(Sx)-KNKK-Z (SEQ ID NO: 190), X-KGTLVQTKGTGASGS*F-C(Sx)-LNKK-Z (SEQ ID NO: 191), X-RRR-C(Sx)-SS*LRA-Z (SEQ ID NO: 192), X-RR-C(Sx)-SS*LRA-Z (SEQ ID NO: 193), X-RRLSS*L-C(Sx)-A-Z (SEQ ID NO: 194), X-RT-C(Sx)-RS*GSVYEPLKI-Z (SEQ ID NO: 195), X-RFAVRDMRQT*V-C(Sx)-VGVIKAVDKK-Z (SEQ ID NO: 196), X-AAKI-C(Sx)-AS*FRGHMARKK-Z (SEQ ID NO: 197), X-RR-C(Sx)-RT*GRGRRGIFR-Z (SEQ ID NO: 198), X-QK-C(Sx)-PS*QRSKYL-Z (SEQ ID NO: 199), X-STASQDVANRFARKGS*L-C(Sx)-QKNV-Z (SEQ ID NO: 200), X-MFAVRDRRQT*V-C(Sx)-KGVIKAVDAV-Z (SEQ ID NO: 201), X-RLGRDK-C(Sx)-KT*LRQIRQ-Z (SEQ ID NO: 202), X-RLGRDKYKT*L-C(Sx)-QIRQ-Z (SEQ ID NO: 203), X-ERMRPRKRQGS*V-C(Sx)RRV-Z (SEQ ID NO: 204), X-HAT*P-C(Sx)-KKKRK-Z (SEQ ID NO: 205), X-GGG-C(Sx)-AT*PKKAKKL-Z (SEQ ID NO: 206), X-RRRFRPAS*P-C(Sx)-RGPPK-Z (SEQ ID NO: 208), X-RRRFRPAS*P-C(Sx)-RGPPK-Z (SEQ ID NO: 208), X-IPTS*P-C(Sx)-TTTYFFF-Z (SEQ ID NO: 209), X-I-C(Sx)-TS*PITTTYFFF-Z (SEQ ID NO: 210), X-IPTS*P-C(Sx)-TTTYFFFKKK-Z (SEQ ID NO: 211), and X-GGG-C(Sx)-AT*PKKAKKL-Z (SEQ ID NO: 206); or
a compound selected from the group consisting of: X-DANPLMA-C(Sx)-GT*LTRRHQNGRF-Z (SEQ ID NO: 1200), X-DANPLMA-C(Sx)-GT*LTRRHANGRF-Z (SEQ ID NO: 1534), X-DANPLMA-C(Sx)-GT*LTRRHDNGRF-Z (SEQ ID NO: 1535), X-DANPLMA-C(Sx)-GT*LTRRHENGRF-Z (SEQ ID NO: 1536), X-DANPLMA-C(Sx)-GT*LTRRHFNGRF-Z (SEQ ID NO: 1537), X-DANPLMA-C(Sx)-GT*LTRRHGNGRF-Z (SEQ ID NO: 1538), X-DANPLMA-C(Sx)-GT*LTRRHHNGRF-Z (SEQ ID NO: 1539), X-DANPLMA-C(Sx)-GT*LTRRHINGRF-Z (SEQ ID NO: 1540), X-DANPLMA-C(Sx)-GT*LTRRHKNGRF-Z (SEQ ID NO: 1541), X-DANPLMA-C(Sx)-GT*LTRRHLNGRF-Z (SEQ ID NO: 1542), X-DANPLMA-C(Sx)-GT*LTRRHNNGRF-Z (SEQ ID NO: 1543), X-DANPLMA-C(Sx)-GT*LTRRHPNGRF-Z (SEQ ID NO: 1544), X-DANPLMA-C(Sx)-GT*LTRRHQ[Nle]GRF-Z (SEQ ID NO: 1545), X-DANPLMA-C(Sx)-GT*LTRRHQAGRF-Z (SEQ ID NO: 1546), X-DANPLMA-C(Sx)-GT*LTRRHQDGRF-Z (SEQ ID NO: 1547), X-DANPLMA-C(Sx)-GT*LTRRHQEGRF-Z (SEQ ID NO: 1548), X-DANPLMA-C(Sx)-GT*LTRRHQFGRF-Z (SEQ ID NO: 1549), X-DANPLMA-C(Sx)-GT*LTRRHQGGRF-Z (SEQ ID NO: 1550), X-DANPLMA-C(Sx)-GT*LTRRHQHGRF-Z (SEQ ID NO: 1551), X-DANPLMA-C(Sx)-GT*LTRRHQIGRF-Z (SEQ ID NO: 1552), X-DANPLMA-C(Sx)-GT*LTRRHQKGRF-Z (SEQ ID NO: 1553), X-DANPLMA-C(Sx)-GT*LTRRHQLGRF-Z (SEQ ID NO: 1554), X-DANPLMA-C(Sx)-GT*LTRRHQN[Nle]RF-Z (SEQ ID NO: 1555), X-DANPLMA-C(Sx)-GT*LTRRHQNARF-Z (SEQ ID NO: 1556), X-DANPLMA-C(Sx)-GT*LTRRHQNDRF-Z (SEQ ID NO: 1557), X-DANPLMA-C(Sx)-GT*LTRRHQNERF-Z (SEQ ID NO: 1558), X-DANPLMA-C(Sx)-GT*LTRRHQNFRF-Z (SEQ ID NO: 1559), X-DANPLMA-C(Sx)-GT*LTRRHQNG[Nle]F-Z (SEQ ID NO: 1560), X-DANPLMA-C(Sx)-GT*LTRRHQNGAF-Z (SEQ ID NO: 1561), X-DANPLMA-C(Sx)-GT*LTRRHQNGDF-Z (SEQ ID NO: 1562), X-DANPLMA-C(Sx)-GT*LTRRHQNGEF-Z (SEQ ID NO: 1563), X-DANPLMA-C(Sx)-GT*LTRRHQNGFF-Z (SEQ ID NO: 1564), X-DANPLMA-C(Sx)-GT*LTRRHQNGGF-Z (SEQ ID NO: 1565), X-DANPLMA-C(Sx)-GT*LTRRHQNGHF-Z (SEQ ID NO: 1566), X-DANPLMA-C(Sx)-GT*LTRRHQNGIF-Z (SEQ ID NO: 1567), X-DANPLMA-C(Sx)-GT*LTRRHQNGKF-Z (SEQ ID NO: 1568), X-DANPLMA-C(Sx)-GT*LTRRHQNGLF-Z (SEQ ID NO: 1569), X-DANPLMA-C(Sx)-GT*LTRRHQNGNF-Z (SEQ ID NO: 1570), X-DANPLMA-C(Sx)-GT*LTRRHQNGPF-Z (SEQ ID NO: 1571), X-DANPLMA-C(Sx)-GT*LTRRHQNGQF-Z (SEQ ID NO: 1572), X-DANPLMA-C(Sx)-GT*LTRRHQNGR[Nle]-Z (SEQ ID NO: 1573), X-DANPLMA-C(Sx)-GT*LTRRHQNGRA-Z (SEQ ID NO: 1574), X-DANPLMA-C(Sx)-GT*LTRRHQNGRD-Z (SEQ ID NO: 1575), X-DANPLMA-C(Sx)-GT*LTRRHQNGRE-Z (SEQ ID NO: 1576), X-DANPLMA-C(Sx)-GT*LTRRHQNGRG-Z (SEQ ID NO: 1577), X-DANPLMA-C(Sx)-GT*LTRRHQNGRH-Z (SEQ ID NO: 1578), X-DANPLMA-C(Sx)-GT*LTRRHQNGRI-Z (SEQ ID NO: 1579), X-DANPLMA-C(Sx)-GT*LTRRHQNGRK-Z (SEQ ID NO: 1580), X-DANPLMA-C(Sx)-GT*LTRRHQNGRL-Z (SEQ ID NO: 1581), X-DANPLMA-C(Sx)-GT*LTRRHQNGRN-Z (SEQ ID NO: 1582), and X-DANPLMA-C(Sx)-GT*LTRRHQNGRP-Z (SEQ ID NO: 1583); or
a compound selected from the group consisting of: X-C(Sx)-FS*LRRKAK-Z (SEQ ID NO: 220), X-HMRSAMS*G-C(Sx)-HLVKRR-Z (SEQ ID NO: 213), X-HMRS-C(Sx)-MS*GLHLVKRR-Z (SEQ ID NO: 214), X-AMARAAS*A-C(Sx)-ALARRR-Z (SEQ ID NO: 215), X-KKKALSRQFS*V-C(Sx)-Z (SEQ ID NO: 216), X-GRTGRRNS*I-C(Sx)-Z (SEQ ID NO: 217), X-FLAKRRRLSS*L-C(Sx)-A-Z (SEQ ID NO: 218), X-KKRNRTLT*V-C(Sx)-Z (SEQ ID NO: 219), X-KEIYNT*I-C(Sx)-Z (SEQ ID NO: 221), X-RKIS*A-C(Sx)-EFDRPLR-Z (SEQ ID NO: 222), X-R-C(Sx)-IS*ASEFDRPLR-Z (SEQ ID NO: 223), X-RKIS*A-C(Sx)-EFDRPLR-Z (SEQ ID NO: 222), X-RKRS*R-C(Sx)-E-Z (SEQ ID NO: 224), X-RARTLS*F-C(Sx)-EPG-Z (SEQ ID NO: 225), X-KRRE-C(Sx)-LS*RRP[pS]YR-Z (SEQ ID NO: 226), X-RRAAEE-C(Sx)-DS*RAG[pS]PQL-Z (SEQ ID NO: 227), X-RYR-C(Sx)-PT*GMYY-Z (SEQ ID NO: 228), X-RYRHPT*R-C(Sx)-YY-Z (SEQ ID NO: 229), X-KPDRKKRY*TV-C(Sx)-GNPY-Z (SEQ ID NO: 230), X-RRRLS*F-C(Sx)-EPG-Z (SEQ ID NO: 231), X-PL-C(Sx)-RT*LSVAGLPGKK-Z (SEQ ID NO: 232), X-KKLNRT*L-C(Sx)-VA-Z (SEQ ID NO: 233), X-KKALRRQET*V-C(Sx)-AL-Z (SEQ ID NO: 234), X-LSNLYHQGKFLQT*F-C(Sx)-GSPLYRRR-Z (SEQ ID NO: 235), X-RISDELMDAT*F-C(Sx)-DQEAK-Z (SEQ ID NO: 236), X-DELMEFS*F-C(Sx)-DQEAKV-Z (SEQ ID NO: 237), X-SGEDT*L-C(Sx)-DSDDED-Z (SEQ ID NO: 238), X-GRHD[pS]GLDS*M-C(Sx)-Z (SEQ ID NO: 239), X-KRRRALS[pS]VAS*L-C(Sx)-Z (SEQ ID NO: 240), X-RRKDLHDDEEDEAMS*I-C(Sx)-A-Z (SEQ ID NO: 241), X-RRA-C(Sx)-DS*DDDDD-Z (SEQ ID NO: 242), X-LGKEF-C(Sx)-RS*YRLRYSRDGRR-Z (SEQ ID NO: 243), X-LGKEFSRS*Y-C(Sx)LRYSRDGRR-Z (SEQ ID NO: 244), X-GRPRTSS*F-C(Sx)-EG-Z (SEQ ID NO: 245), X-PRTSS*F-C(Sx)-E-Z (SEQ ID NO: 246), X-KRRRLAS*L-C(Sx)-A-Z (SEQ ID NO: 247), X-KKRNRTLS*V-C(Sx)-Z (SEQ ID NO: 248), X-RKKFGES-C(Sx)-KT*KTKEFL-Z (SEQ ID NO: 249), and X-RKKFGESEKT*K-C(Sx)-KEFL-Z (SEQ ID NO: 250); or
a compound selected from the group consisting of: X-RLPWWR-C(Sx)-YT*WVRARQVNTKQR-Z (SEQ ID NO: 4513), X-RLPWWR-C(Sx)-YT*WQVERQGNTKQR-Z (SEQ ID NO: 4482), X-RLPWWR-C(Sx)-YT*WQVERQVNTKQR-Z (SEQ ID NO: 4483), X-RLPWWR-C(Sx)-YT*WRRARDGNTKQR-Z (SEQ ID NO: 4484), X-RLPWWR-C(Sx)-YT*WRRARDVNTKQR-Z (SEQ ID NO: 4485), X-RLPWWR-C(Sx)-YT*WRRAREGNTKQR-Z (SEQ ID NO: 4486), X-RLPWWR-C(Sx)-YT*WRRAREVNTKQR-Z (SEQ ID NO: 4487), X-RLPWWR-C(Sx)-YT*WRRARQGNTKQR-Z (SEQ ID NO: 4488), X-RLPWWR-C(Sx)-YT*WRRARQVNTKQR-Z (SEQ ID NO: 4489), X-RLPWWR-C(Sx)-YT*WRRERDGNTKQR-Z (SEQ ID NO: 4490), X-RLPWWR-C(Sx)-YT*WRRERDVNTKQR-Z (SEQ ID NO: 4491), X-RLPWWR-C(Sx)-YT*WRREREGNTKQR-Z (SEQ ID NO: 4492), X-RLPWWR-C(Sx)-YT*WRREREVNTKQR-Z (SEQ ID NO: 4493), X-RLPWWR-C(Sx)-YT*WRRERQGNTKQR-Z (SEQ ID NO: 4494), X-RLPWWR-C(Sx)-YT*WRRERQVNTKQR-Z (SEQ ID NO: 4495), X-RLPWWR-C(Sx)-YT*WRVARDGNTKQR-Z (SEQ ID NO: 4496), X-RLPWWR-C(Sx)-YT*WRVARDVNTKQR-Z (SEQ ID NO: 4497), X-RLPWWR-C(Sx)-YT*WRVAREGNTKQR-Z (SEQ ID NO: 4498), X-RLPWWR-C(Sx)-YT*WRVAREVNTKQR-Z (SEQ ID NO: 4499), X-RLPWWR-C(Sx)-YT*WRVARQGNTKQR-Z (SEQ ID NO: 4500), X-RLPWWR-C(Sx)-YT*WRVARQVNTKQR-Z (SEQ ID NO: 4501), X-RLPWWR-C(Sx)-YT*WRVERDGNTKQR-Z (SEQ ID NO: 4502), X-RLPWWR-C(Sx)-YT*WRVERDVNTKQR-Z (SEQ ID NO: 4503), X-RLPWWR-C(Sx)-YT*WRVEREGNTKQR-Z (SEQ ID NO: 4504), X-RLPWWR-C(Sx)-YT*WRVEREVNTKQR-Z (SEQ ID NO: 4505), X-RLPWWR-C(Sx)-YT*WRVERQGNTKQR-Z (SEQ ID NO: 4506), X-RLPWWR-C(Sx)-YT*WRVERQVNTKQR-Z (SEQ ID NO: 4507), X-RLPWWR-C(Sx)-YT*WVRARDGNTKQR-Z (SEQ ID NO: 4508), X-RLPWWR-C(Sx)-YT*WVRARDVNTKQR-Z (SEQ ID NO: 4509), X-RLPWWR-C(Sx)-YT*WVRAREGNTKQR-Z (SEQ ID NO: 4510), X-RLPWWR-C(Sx)-YT*WVRAREVNTKQR-Z (SEQ ID NO: 4511), X-RLPWWR-C(Sx)-YT*WVRARQGNTKQR-Z (SEQ ID NO: 4512), X-RLPWWR-C(Sx)-YT*WVRERDGNTKQR-Z (SEQ ID NO: 4514), X-RLPWWR-C(Sx)-YT*WVRERDVNTKQR-Z (SEQ ID NO: 4515), X-RLPWWR-C(Sx)-YT*WVREREGNTKQR-Z (SEQ ID NO: 4516), X-RLPWWR-C(Sx)-YT*WVREREVNTKQR-Z (SEQ ID NO: 4517), X-RLPWWR-C(Sx)-YT*WVRERQGNTKQR-Z (SEQ ID NO: 4518), X-RLPWWR-C(Sx)-YT*WVRERQVNTKQR-Z (SEQ ID NO: 4519), X-RLPWWR-C(Sx)-YT*WVVARDGNTKQR-Z (SEQ ID NO: 4520), X-RLPWWR-C(Sx)-YT*WVVARDVNTKQR-Z (SEQ ID NO: 4521), X-RLPWWR-C(Sx)-YT*WVVAREGNTKQR-Z (SEQ ID NO: 4522), X-RLPWWR-C(Sx)-YT*WVVAREVNTKQR-Z (SEQ ID NO: 4523), X-RLPWWR-C(Sx)-YT*WVVARQGNTKQR-Z (SEQ ID NO: 4524), X-RLPWWR-C(Sx)-YT*WVVARQVNTKQR-Z (SEQ ID NO: 4525), X-RLPWWR-C(Sx)-YT*WVVERDGNTKQR-Z (SEQ ID NO: 4526), X-RLPWWR-C(Sx)-YT*WVVERDVNTKQR-Z (SEQ ID NO: 4527), X-RLPWWR-C(Sx)-YT*WVVEREGNTKQR-Z (SEQ ID NO: 4528), X-RLPWWR-C(Sx)-YT*WVVEREVNTKQR-Z (SEQ ID NO: 4529), X-RLPWWR-C(Sx)-YT*WVVERQGNTKQR-Z (SEQ ID NO: 4530), and X-RLPWWR-C(Sx)-YT*WVVERQVNTKQR-Z (SEQ ID NO: 4531); or
a compound selected from the group consisting of: X-LKP-C(Sx)-AS*FKKFA-Z (SEQ ID NO: 5485), X-[Nle]KR-C(Sx)-AS*FKKFA-Z (SEQ ID NO: 5457); X-AKR-C(Sx)-AS*FKKFA-Z (SEQ ID NO: 5458); X-DKR-C(Sx)-AS*FKKFA-Z (SEQ ID NO: 5459); X-EKR-C(Sx)-AS*FKKFA-Z (SEQ ID NO: 5460); X-FKR-C(Sx)-AS*FKKFA-Z (SEQ ID NO: 5461); X-GKR-C(Sx)-AS*FKKFA-Z (SEQ ID NO: 5462); X-HKR-C(Sx)-AS*FKKFA-Z (SEQ ID NO: 5463); X-IKR-C(Sx)-AS*FKKFA-Z (SEQ ID NO: 5464); X-KKR-C(Sx)-AS*FKKFA-Z (SEQ ID NO: 5465); X-L[Nle]R-C(Sx)-AS*FKKFA-Z (SEQ ID NO: 5466); X-LAR-C(Sx)-AS*FKKFA-Z (SEQ ID NO: 5467); X-LDR-C(Sx)-AS*FKKFA-Z (SEQ ID NO: 5468); X-LER-C(Sx)-AS*FKKFA-Z (SEQ ID NO: 5469); X-LFR-C(Sx)-AS*FKKFA-Z (SEQ ID NO: 5470); X-LGR-C(Sx)-AS*FKKFA-Z (SEQ ID NO: 5471); X-LHR-C(Sx)-AS*FKKFA-Z (SEQ ID NO: 5472); X-LIR-C(Sx)-AS*FKKFA-Z (SEQ ID NO: 5473); X-LK[Nle]-C(Sx)-AS*FKKFA-Z (SEQ ID NO: 5474); X-LKA-C(Sx)-AS*FKKFA-Z (SEQ ID NO: 5475); X-LKD-C(Sx)-AS*FKKFA-Z (SEQ ID NO: 5476); X-LKE-C(Sx)-AS*FKKFA-Z (SEQ ID NO: 5477); X-LKF-C(Sx)-AS*FKKFA-Z (SEQ ID NO: 5478); X-LKG-C(Sx)-AS*FKKFA-Z (SEQ ID NO: 5479); X-LKH-C(Sx)-AS*FKKFA-Z (SEQ ID NO: 5480); X-LKI-C(Sx)-AS*FKKFA-Z (SEQ ID NO: 5481); X-LKK-C(Sx)-AS*FKKFA-Z (SEQ ID NO: 5482); X-LKL-C(Sx)-AS*FKKFA-Z (SEQ ID NO: 5483); X-LKN-C(Sx)-AS*FKKFA-Z (SEQ ID NO: 5484); X-LKQ-C(Sx)-AS*FKKFA-Z (SEQ ID NO: 5486); or
a compound selected from the group consisting of: X-KKKKVKKRPQRAHS*D-C(Sx)-FS-Z (SEQ ID NO: 3470), X-KKKKVKKRPQRAES*D-C(Sx)-FA-Z (SEQ ID NO: 3463), X-KKKKVKKRPQRAES*D-C(Sx)-FS-Z (SEQ ID NO: 3464), X-KKKKVKKRPQRAES*N-C(Sx)-FA-Z (SEQ ID NO: 3465), X-KKKKVKKRPQRAES*N-C(Sx)-FS-Z (SEQ ID NO: 3466), X-KKKKVKKRPQRAESD-C(Sx)-FS*-Z (SEQ ID NO: 3467), X-KKKKVKKRPQRAESN-C(Sx)-FS*-Z (SEQ ID NO: 3468), X-KKKKVKKRPQRAHS*D-C(Sx)-FA-Z (SEQ ID NO: 3469), X-KKKKVKKRPQRAHS*N-C(Sx)-FA-Z (SEQ ID NO: 3471), X-KKKKVKKRPQRAHS*N-C(Sx)-FS-Z (SEQ ID NO: 3472), X-KKKKVKKRPQRAHSD-C(Sx)-FS*-Z (SEQ ID NO: 3473), X-KKKKVKKRPQRAHSN-C(Sx)-FS*-Z (SEQ ID NO: 3474), X-KKKKVKKRPQRATS*D-C(Sx)-FA-Z (SEQ ID NO: 3475), X-KKKKVKKRPQRATS*D-C(Sx)-FS-Z (SEQ ID NO: 3476), X-KKKKVKKRPQRATS*N-C(Sx)-FA-Z (SEQ ID NO: 3477), X-KKKKVKKRPQRATS*N-C(Sx)-FS-Z (SEQ ID NO: 3478), X-KKKKVKKRPQRATSD-C(Sx)-FS*-Z (SEQ ID NO: 3479), X-KKKKVKKRPQRATSN-C(Sx)-FS*-Z (SEQ ID NO: 3480), X-KKKLAPNPSFS*P-C(Sx)-PGFTP-Z (SEQ ID NO: 1102), X-KKKLIDS[Nle]ANS*F-C(Sx)-GTRSY-Z (SEQ ID NO: 3481), X-KKKLIDS[Nle]ANSF-C(Sx)-GT*RSY-Z (SEQ ID NO: 3482), X-KKKLPPVFSGT*P-C(Sx)-GSGAG-Z (SEQ ID NO: 3483), X-KKKLPPVFSGTP-C(Sx)-GS*GAG-Z (SEQ ID NO: 3484), X-KKKLSNSQGQS*P-C(Sx)-VPFPA-Z (SEQ ID NO: 3485), X-KKKLTDPRLLS*P-C(Sx)-QPALQ-Z (SEQ ID NO: 3486), X-KKKNVH[Nle]VSTT*L-C(Sx)-VDSR-Z (SEQ ID NO: 3487), X-KKKPLSPFPVS*P-C(Sx)-AGPGS-Z (SEQ ID NO: 3488), X-KKKPQPLRS*P-C(Sx)-LDNPT-Z (SEQ ID NO: 3489), X-KKKPQRAT*S-C(Sx)-VFAMF-Z (SEQ ID NO: 3490), X-KKKPSAPA-C(Sx)-ST*PGIRDSA-Z (SEQ ID NO: 3491), X-KKKPSAPALST*P-C(Sx)-IRDSA-Z (SEQ ID NO: 3492), X-KKKPSAPALST*PG-C(Sx)-RDSA-Z (SEQ ID NO: 3493), X-KKKQ[Nle]AGTPIT*P-C(Sx)-KDGFT-Z (SEQ ID NO: 3494), X-KKKQAATGFGS*P-C(Sx)-QYSAD-Z (SEQ ID NO: 3495), X-KKKQGPAPVGT*P-C(Sx)-FNRQH-Z (SEQ ID NO: 3496), X-KKKQLSKWPGS*P-C(Sx)-SRSSD-Z (SEQ ID NO: 3497), X-KKKQNRSGAMS*P-C(Sx)-SWNSD-Z (SEQ ID NO: 3498), X-KKKQS[Nle]VVPQT*P-C(Sx)-HTARV-Z (SEQ ID NO: 3499), X-KKKQS[Nle]VVPQTP-C(Sx)-HT*ARV-Z (SEQ ID NO: 3500), X-KKKQSAQS-C(Sx)-AT*PVVSVAT-Z (SEQ ID NO: 3501), X-KKKQSAQSLAT*P-C(Sx)-VSVAT-Z (SEQ ID NO: 3502), X-KKKQSAQSLATP-C(Sx)-VS*VAT-Z (SEQ ID NO: 3503), X-KKKRPASVNGS*P-C(Sx)-ATSES-Z (SEQ ID NO: 3504), X-KKKRPASVNGSP-C(Sx)-AT*SES-Z (SEQ ID NO: 3505), X-KKKS[Nle]DGLFGT*I-C(Sx)-YLP-Z (SEQ ID NO: 3506), X-KKKSALQG-C(Sx)-NS*PG[Nle]LSLG-Z (SEQ ID NO: 3507), X-KKKSALQGFNS*P-C(Sx)-[Nle]LSLG-Z (SEQ ID NO: 3508), X-KKKSASGSAFS*P-C(Sx)-PGSAA-Z (SEQ ID NO: 3509), X-KKKSAVNGTSS*A-C(Sx)-TNLEA-Z (SEQ ID NO: 3510), and X-KKKSGYS*S-C(Sx)-GSPGTPGSR-Z (SEQ ID NO: 3511); or
a compound selected from the group consisting of: X-SR-C(Sx)-SS*PHQ[pS]EDEEE-Z (SEQ ID NO: 4741), X-S*R-C(Sx)-LSVSSLPGLED-Z (SEQ ID NO: 4696), X-S*R-C(Sx)-SSPHQ[pS]EDEEE-Z (SEQ ID NO: 4697), X-SAKS*R-C(Sx)-QTAPVP[Nle]PD-Z (SEQ ID NO: 4698), X-SAKSR-C(Sx)-QT*APVP[Nle]PD-Z (SEQ ID NO: 4699), X-SAKSRLQT*A-C(Sx)-VP[Nle]PD-Z (SEQ ID NO: 4700), X-SANLLS*P-C(Sx)-PA-Z (SEQ ID NO: 4701), X-SEGLS*M-C(Sx)-NYIGLINRIKK-Z (SEQ ID NO: 4702), X-SFITPPTT*P-C(Sx)-LRRHT-Z (SEQ ID NO: 1092), X-SGDED-C(Sx)-SS*IADMDFSAL-Z (SEQ ID NO: 4703), X-SGDEDFSS*I-C(Sx)-DMDFSALLSQ-Z (SEQ ID NO: 4704), X-SGDY[Nle]P[Nle]S*P-C(Sx)-SVSAP-Z (SEQ ID NO: 4705), X-SGLS*R-C(Sx)-DSPEPFGHY-Z (SEQ ID NO: 4706), X-SGLSR-C(Sx)-DS*PEPFGHY-Z (SEQ ID NO: 4707), X-SGLSRRDS*P-C(Sx)-PFGHY-Z (SEQ ID NO: 4708), X-SGQL-C(Sx)-DS*[Nle]ANSFV-Z (SEQ ID NO: 4709), X-SGQLIDS*[Nle]-C(Sx)-NSFV-Z (SEQ ID NO: 4710), X-SGQLIDS[Nle]ANS*F-C(Sx)-G-Z (SEQ ID NO: 4711), X-SGQLIDS[Nle]-C(Sx)-NS*FV-Z (SEQ ID NO: 4712), X-SGRPR-C(Sx)-TS*FAESSKP-Z (SEQ ID NO: 4713), X-SGRPRTTS*F-C(Sx)-ESSKP-Z (SEQ ID NO: 4714), X-SGVEV-C(Sx)-VT*PTRTEII-Z (SEQ ID NO: 1061), X-SGVEVRVT*P-C(Sx)-RTEII-Z (SEQ ID NO: 1060), X-SGVEVRVTP-C(Sx)-RT*EII-Z (SEQ ID NO: 4715), X-SIKS*E-C(Sx)-ISPPRDR[Nle]T-Z (SEQ ID NO: 4716), X-SIKSE-C(Sx)-IS*PPRDR[Nle]T-Z (SEQ ID NO: 4717), X-SIKSEPIS*P-C(Sx)-RDR[Nle]T-Z (SEQ ID NO: 4718), X-SIMQQ-C(Sx)-DS*PHVVKYYKKK-Z (SEQ ID NO: 4719), X-SKS*S-C(Sx)-NGTSGDREDK-Z (SEQ ID NO: 4720), X-SLD-C(Sx)-SS*LSLSLSSANSLYDD-Z (SEQ ID NO: 4721), X-SLDSSSLS*L-C(Sx)-LSSANSLYDD-Z (SEQ ID NO: 4722), X-SLDSSSLSL-C(Sx)-LS*SANSLYDD-Z (SEQ ID NO: 4723), X-SNGRR-C(Sx)-RS*PTQSFRF-Z (SEQ ID NO: 1037), X-SNGRRKRS*P-C(Sx)-QSFRF-Z (SEQ ID NO: 1036), X-SNGRRKRSP-C(Sx)-QS*FRF-Z (SEQ ID NO: 4724), X-SNSLKKSS*A-C(Sx)-LKKIL-Z (SEQ ID NO: 1180), X-SNT*GQ-C(Sx)-ISPG[Nle]KRRI-Z (SEQ ID NO: 4725), X-SNTGQAIS*P-C(Sx)-[Nle]KRRI-Z (SEQ ID NO: 4726), X-SNTGQAIS*PG-C(Sx)-KRRI-Z (SEQ ID NO: 4727), X-SNTGQ-C(Sx)-IS*PG[Nle]KRRI-Z (SEQ ID NO: 4728), X-SPAR-C(Sx)-SS*YSEANEP-Z (SEQ ID NO: 4729), X-SPLSDPSS*P-C(Sx)-ASEDF-Z (SEQ ID NO: 1182), X-SPLSDPSSP-C(Sx)-AS*EDF-Z (SEQ ID NO: 4730), X-SPS-C(Sx)-RS*RSRSRSRSPGRPSK-Z (SEQ ID NO: 4731), X-SPSRRS*R-C(Sx)-RSRSRSPGRPSK-Z (SEQ ID NO: 4732), X-SPSRRSR-C(Sx)-RS*RSRSPGRPSK-Z (SEQ ID NO: 4733), X-SPSRRSRS*R-C(Sx)-RSRSPGRPSK-Z (SEQ ID NO: 4734), X-SPSRRSRSR-C(Sx)-RS*RSPGRPSK-Z (SEQ ID NO: 4735), X-SPSRRSRSRS*R-C(Sx)-RSPGRPSK-Z (SEQ ID NO: 4736), X-SPSRRSRSRSR-C(Sx)-RS*PGRPSK-Z (SEQ ID NO: 4737), X-SPSRRSRSRSRS*R-C(Sx)-PGRPSK-Z (SEQ ID NO: 4738), X-SPSRRSRSRSRSRS*P-C(Sx)-RPSK-Z (SEQ ID NO: 4739), X-SR-C(Sx)-LS*VSSLPGLED-Z (SEQ ID NO: 4740), X-SRDPVART*S-C(Sx)-LQTPA-Z (SEQ ID NO: 1104), X-SRLRD-C(Sx)-PS*APLEAPE-Z (SEQ ID NO: 1171), X-SRRS*G-C(Sx)-SSPSYVAVT-Z (SEQ ID NO: 4742), X-SRRSG-C(Sx)-SS*PSYVAVT-Z (SEQ ID NO: 1063), X-SRRSGLSS*P-C(Sx)-YVAVT-Z (SEQ ID NO: 1062), X-SRSHSAKT*P-C(Sx)-FSVQS-Z (SEQ ID NO: 1069), X-SRSHSAKT*PG-C(Sx)-SVQS-Z (SEQ ID NO: 4743), X-SRSHSAKTP-C(Sx)-FS*VQS-Z (SEQ ID NO: 4744), X-SRSHS-C(Sx)-KT*PGFSVQS-Z (SEQ ID NO: 4745), X-SRSRS*R-C(Sx)-RAPGRPSKG-Z (SEQ ID NO: 4746), X-SRSRS*R-C(Sx)-RGPGRPSKG-Z (SEQ ID NO: 4747), X-SRSRS*R-C(Sx)-RSPGRPSKG-Z (SEQ ID NO: 4748), X-SRSRSNSRSRS*Y-C(Sx)-PRRSRGS-Z (SEQ ID NO: 4749), X-SRSRSNSRSRSYS*P-C(Sx)-RSRGS-Z (SEQ ID NO: 4750), X-SRSRSNSRSRSYSP-C(Sx)-RS*RGS-Z (SEQ ID NO: 4751), X-SRSRSR-C(Sx)-RS*PGRPSKG-Z (SEQ ID NO: 4752), X-SRSRSRSNSRSRSYS*P-C(Sx)-RSR-Z (SEQ ID NO: 4753), X-SRSRSRSNSRSRSYSP-C(Sx)-RS*R-Z (SEQ ID NO: 4754), X-SRSRSRSRS*P-C(Sx)-RPSKGR-Z (SEQ ID NO: 4755), X-SRSRSRSRS*P-C(Sx)-RPSKG-Z (SEQ ID NO: 4756), X-SRSRSRSRSNSRSRS*Y-C(Sx)-PRR-Z (SEQ ID NO: 4757), X-SRSRSRSRSNSRSRSYS*P-C(Sx)-R-Z (SEQ ID NO: 4758), X-SSKIRRLS*A-C(Sx)-KQQ-Z (SEQ ID NO: 4759), X-SSKRAKAKTTKKRPQRAT*S-C(Sx)-VFS-Z (SEQ ID NO: 4760), X-SSKRAKAKTTKKRPQRATS*N-C(Sx)-FS-Z (SEQ ID NO: 998), X-SSKRAKAKTTKKRPQRATSN-C(Sx)-FS*-Z (SEQ ID NO: 4761), X-SSLET-C(Sx)-ST*QELYSIP-Z (SEQ ID NO: 4762), X-SSPS-C(Sx)-RS*RSRSRSRS-Z (SEQ ID NO: 4763), X-SSPSRR-C(Sx)-RS*RSRSRS-Z (SEQ ID NO: 4764), X-SSPSRRS*R-C(Sx)-RSRSRS-Z (SEQ ID NO: 4765), X-SSPSRRSR-C(Sx)-RS*RSRS-Z (SEQ ID NO: 4766), X-SSPSRRSRSRS*R-C(Sx)-RA-Z (SEQ ID NO: 4767), X-SSPSRRSRSRS*R-C(Sx)-RG-Z (SEQ ID NO: 4768), X-SSPSRRSRSRS*R-C(Sx)-RS-Z (SEQ ID NO: 4769), X-SSPSRRSRSRSR-C(Sx)-RS*-Z (SEQ ID NO: 4770), and X-SSPSRRSRSRSRS*R-C(Sx)-Z (SEQ ID NO: 4771); or
a compound selected from the group consisting of: X-KKSR-C(Sx)-DY*[Nle]T[Nle]QIG-Z (SEQ ID NO: 5051), X-KKKQAATGFGSP-C(Sx)-QY*SAD-Z (SEQ ID NO: 5048), X-KKKRAHEEIY*F-C(Sx)-FWG-Z (SEQ ID NO: 916), X-KKKSNPALLSSP-C(Sx)-YY*SAA-Z (SEQ ID NO: 5049), X-KKKTSF[Nle][Nle][pT]PY*V-C(Sx)-TRY-Z (SEQ ID NO: 5050), X-KKTNSSEGLSMGNY*I-C(Sx)-LINR-Z (SEQ ID NO: 5052), X-KVSETDDY*A-C(Sx)-IIDEE-Z (SEQ ID NO: 5053), X-LEEEEY*I-C(Sx)-Z (SEQ ID NO: 5054), X-LEEEY*I-C(Sx)-IV-Z (SEQ ID NO: 5055), X-LHQEDNDY*I-C(Sx)-ASLIK-Z (SEQ ID NO: 969), X-LHQEDNDY*IN-C(Sx)-SLIK-Z (SEQ ID NO: 970), X-LMLRLQDY*E-C(Sx)-KTKKA-Z (SEQ ID NO: 951), X-LSRDLIMK-C(Sx)-DY*ELVSTKPTR-Z (SEQ ID NO: 5056), X-LSRDLIMKEDY*E-C(Sx)-VSTKPTR-Z (SEQ ID NO: 5057), X-LSRDLIMKEDY*E-C(Sx)-VSTK-Z (SEQ ID NO: 5058), X-LVSETDDY*A-C(Sx)-IIDEE-Z (SEQ ID NO: 5059), X-MAEEKKAY*K-C(Sx)-AKERE-Z (SEQ ID NO: 932), X-MEEEEY*I-C(Sx)-Z (SEQ ID NO: 5060), X-MEEEY*I-C(Sx)-IV-Z (SEQ ID NO: 5061), X-MK-C(Sx)-DY*ELVSTKPTRTSKVR-Z (SEQ ID NO: 5062), X-MQDNS-C(Sx)-TY*GKIWEGS-Z (SEQ ID NO: 963), X-MRHVSISY*D-C(Sx)-PPTPG-Z (SEQ ID NO: 953), X-NEEEEY*I-C(Sx)-Z (SEQ ID NO: 5063), X-NEEEY*I-C(Sx)-IV-Z (SEQ ID NO: 5064), X-NFANFSAY*P-C(Sx)-EEDMI-Z (SEQ ID NO: 949), X-NHIG-C(Sx)-TGY*LNTVTVSAKKK-Z (SEQ ID NO: 5065), X-NKVDD-C(Sx)-NY*AIKRIRL-Z (SEQ ID NO: 5066), X-NKVDDCNY*A-C(Sx)-KRIRL-Z (SEQ ID NO: 5067), X-NVSETDDY*A-C(Sx)-IIDEE-Z (SEQ ID NO: 5068), X-PAFDNLYY*W-C(Sx)-QDPPE-Z (SEQ ID NO: 990), X-PEEEEY*I-C(Sx)-Z (SEQ ID NO: 5069), X-PEEEY*I-C(Sx)-IV-Z (SEQ ID NO: 5070), X-PTAENPEY*L-C(Sx)-LDVPV-Z (SEQ ID NO: 991), X-PTGEAPTY*V-C(Sx)-TQQIP-Z (SEQ ID NO: 983), X-PVPEY*I-C(Sx)-QSV-Z (SEQ ID NO: 941), X-PVSETDDY*A-C(Sx)-IIDEE-Z (SEQ ID NO: 5071), X-QALDNPEY*H-C(Sx)-ASNGP-Z (SEQ ID NO: 992), X-QEEEEY*I-C(Sx)-Z (SEQ ID NO: 5072), X-QEEEY*I-C(Sx)-IV-Z (SEQ ID NO: 5073), X-QLAAK-C(Sx)-AY*LQILSEE-Z (SEQ ID NO: 5074), X-QLAAKLAY*L-C(Sx)-ILSEE-Z (SEQ ID NO: 5075), X-QLTFALRY*L-C(Sx)-FFTKA-Z (SEQ ID NO: 961), X-QVSETDDY*A-C(Sx)-IIDEE-Z (SEQ ID NO: 5076), X-RAEEEEY*I-C(Sx)-LVA-Z (SEQ ID NO: 909), X-RAHEEIY*F-C(Sx)-FWG-Z (SEQ ID NO: 924), X-R-C(Sx)-DY*[Nle]TMQIGKK-Z (SEQ ID NO: 5077), X-REEEEY*I-C(Sx)-Z (SEQ ID NO: 5078), X-REEEY*I-C(Sx)-IV-Z (SEQ ID NO: 5079), X-RLDGE-C(Sx)-IY*IRHSNLM-Z (SEQ ID NO: 948), X-RNEEENIY*S-C(Sx)-PHDST-Z (SEQ ID NO: 959), X-RNEGVATY*A-C(Sx)-AVLFR-Z (SEQ ID NO: 937), X-RQGSSDIY*S-C(Sx)-PEGKL-Z (SEQ ID NO: 982), X-RRAEEEEY*I-C(Sx)-LVA-Z (SEQ ID NO: 908), X-RRHY*Y-C(Sx)-DTHTNTYYLRTFGHNTRR-Z (SEQ ID NO: 5080), X-RRHYYYDTHTNTY*Y-C(Sx)-RTFGHNTRR-Z (SEQ ID NO: 5081), X-RRRAEEEEY*I-C(Sx)-LVA-Z (SEQ ID NO: 993), X-RVSETDDY*A-C(Sx)-IIDEE-Z (SEQ ID NO: 5082), X-S[Nle]SETDDY*A-C(Sx)-IIDEE-Z (SEQ ID NO: 5083), X-SASETDDY*A-C(Sx)-IIDEE-Z (SEQ ID NO: 5084), X-SDSETDDY*A-C(Sx)-IIDEE-Z (SEQ ID NO: 5085), X-SEELDENY*V-C(Sx)-MNPNS-Z (SEQ ID NO: 954), X-SEGLSM-C(Sx)-NY*IGLINRIKK-Z (SEQ ID NO: 5086), X-SESETDDY*A-C(Sx)-IIDEE-Z (SEQ ID NO: 5087), X-SFSETDDY*A-C(Sx)-IIDEE-Z (SEQ ID NO: 5088), X-SGSETDDY*A-C(Sx)-IIDEE-Z (SEQ ID NO: 5089), X-SHDSEENY*V-C(Sx)-MNPNL-Z (SEQ ID NO: 956), X-SHSETDDY*A-C(Sx)-IIDEE-Z (SEQ ID NO: 5090), X-SISETDDY*A-C(Sx)-IIDEE-Z (SEQ ID NO: 5091), X-SKSETDDY*A-C(Sx)-IIDEE-Z (SEQ ID NO: 5092), X-SLSETDDY*A-C(Sx)-IIDEE-Z (SEQ ID NO: 5093), X-SNSETDDY*A-C(Sx)-IIDEE-Z (SEQ ID NO: 5094), X-SPAFD-C(Sx)-LY*[pY]WDQDPP-Z (SEQ ID NO: 989), X-SPAFD-C(Sx)-LY*YWDQDPP-Z (SEQ ID NO: 988), and X-SPATDLY*Q-C(Sx)-PPG-Z (SEQ ID NO: 5095); or
a compound selected from the group consisting of: X-GDQDYLS*L-C(Sx)-VG-Z (SEQ ID NO: 651), X-R-C(Sx)-HY*[Nle]TMQIGKK-Z (SEQ ID NO: 5430); X-R-C(Sx)-IY*[Nle]TMQIGKK-Z (SEQ ID NO: 5431); X-R-C(Sx)-KY*[Nle]TMQIGKK-Z (SEQ ID NO: 5432); X-R-C(Sx)-LY*[Nle]TMQIGKK-Z (SEQ ID NO: 5433); X-R-C(Sx)-NY*[Nle]TMQIGKK-Z (SEQ ID NO: 5434); X-R-C(Sx)-PY*[Nle]TMQIGKK-Z (SEQ ID NO: 5435); X-R-C(Sx)-QY*[Nle]TMQIGKK-Z (SEQ ID NO: 5436); X-R-C(Sx)-RY*[Nle]TMQIGKK-Z (SEQ ID NO: 5437); X-R-C(Sx)-VY*[Nle]TMQIGKK-Z (SEQ ID NO: 5438); X-V-C(Sx)-DY*[Nle]TMQIGKK-Z (SEQ ID NO: 5439); X-KKHTDDGYM-C(Sx)-MS*PGVA-Z (SEQ ID NO: 5440); X-KKHT*D-C(Sx)-GYMPMSPGVA-Z (SEQ ID NO: 5441); X-KKHTDDGY[Nle]-C(Sx)-[Nle]S*PGVA-Z (SEQ ID NO: 5442); X-KKHT*D-C(Sx)-GY[Nle]P[Nle]SPGVA-Z (SEQ ID NO: 5443); X-KKKKVKKRPQRAHSD-C(Sx)-FS*-Z (SEQ ID NO: 3473); X-EDFSS*I-C(Sx)-DMDFSALLSQISS (SEQ ID NO: 1720); X-KKRAARATS*-C(Sx)-VFA-Z (SEQ ID NO: 5444); X-ARKRRRHPS*G-C(Sx)-PTA-Z (SEQ ID NO: 1299); X-ARKRRRHPS*G-C(Sx)-PT-Z (SEQ ID NO: 5445); X-ARKRRRHPS*G-C(Sx)-P-Z (SEQ ID NO: 5446); X-ARKRRRHPS*G[dP]-C(Sx)-Z (SEQ ID NO: 5447); X-ARKRRRHPS*GP-C(Sx)-Z (SEQ ID NO: 5448); X-ARKRRRHPS*G-C(Sx)-Z (SEQ ID NO: 5449); X-RRRQFS*L-C(Sx)-Z (SEQ ID NO: 5450); X-KKERLLDDRHDS*G-C(Sx)-DDMKDEE-Z (SEQ ID NO: 5451); X-KKERLLDDRHDS*G-C(Sx)-DEMKDEE-Z (SEQ ID NO: 5452); X-KKERLLDDRHDS*G-C(Sx)-DAMKDEE-Z (SEQ ID NO: 5453); X-KKERLLDDRHDDG-C(Sx)-DS*MKDEE-Z (SEQ ID NO: 5454); X-KKERLLDDRHDEG-C(Sx)-DS*MKDEE-Z (SEQ ID NO: 5455); X-KKERLLDDRHDAG-C(Sx)-DS*MKDEE-Z (SEQ ID NO: 5456); or
a compound selected from the group consisting of: X-KKRAARAT*S-C(Sx)-VFA-Z (SEQ ID NO: 3679), X-KKNPQRAT*S-C(Sx)-VFAMF-Z (SEQ ID NO: 3672), X-KKNVH[Nle]V-C(Sx)-TT*LPVDSRKK-Z (SEQ ID NO: 3673), X-KKPPQRAT*S-C(Sx)-VFAMF-Z (SEQ ID NO: 3674), X-KKPSVNPSIS*P-C(Sx)-HGVAR-Z (SEQ ID NO: 3675), X-KKQPQRAT*S-C(Sx)-VFAMF-Z (SEQ ID NO: 3676), X-KKR[Nle]QRAT*S-C(Sx)-VFAMF-Z (SEQ ID NO: 3677), X-KKRAA-C(Sx)-AT*SDVFA-Z (SEQ ID NO: 3678), X-KKRAARATS*D-C(Sx)-FA-Z (SEQ ID NO: 3680), X-KKRAAR-C(Sx)-TS*DVFA-Z (SEQ ID NO: 3681), X-KKRDQRAT*S-C(Sx)-VFAMF-Z (SEQ ID NO: 3682), X-KKREQRAT*S-C(Sx)-VFAMF-Z (SEQ ID NO: 3683), X-KKRFQRAT*S-C(Sx)-VFAMF-Z (SEQ ID NO: 3684), X-KKRGQRAT*S-C(Sx)-VFAMF-Z (SEQ ID NO: 3685), X-KKRHQRAT*S-C(Sx)-VFAMF-Z (SEQ ID NO: 3686), X-KKRIQRAT*S-C(Sx)-VFAMF-Z (SEQ ID NO: 3687), X-KKRKQRAT*S-C(Sx)-VFAMF-Z (SEQ ID NO: 3688), X-KKRLQRAT*S-C(Sx)-VFAMF-Z (SEQ ID NO: 3689), X-KKRNQRAT*S-C(Sx)-VFAMF-Z (SEQ ID NO: 3690), X-KKRP[Nle]RAT*S-C(Sx)-VFAMF-Z (SEQ ID NO: 3691), X-KKRPDRAT*S-C(Sx)-VFAMF-Z (SEQ ID NO: 3692), X-KKRPERAT*S-C(Sx)-VFAMF-Z (SEQ ID NO: 3693), X-KKRPFRAT*S-C(Sx)-VFAMF-Z (SEQ ID NO: 3694), X-KKRPGRAT*S-C(Sx)-VFAMF-Z (SEQ ID NO: 3695), X-KKRPHRAT*S-C(Sx)-VFAMF-Z (SEQ ID NO: 3696), X-KKRPIRAT*S-C(Sx)-VFAMF-Z (SEQ ID NO: 3697), X-KKRPKRAT*S-C(Sx)-VFAMF-Z (SEQ ID NO: 3698), X-KKRPLRAT*S-C(Sx)-VFAMF-Z (SEQ ID NO: 3699), X-KKRPNRAT*S-C(Sx)-VFAMF-Z (SEQ ID NO: 3700), X-KKRPPRAT*S-C(Sx)-VFAMF-Z (SEQ ID NO: 3701), X-KKRPQ[Nle]AT*S-C(Sx)-VFAMF-Z (SEQ ID NO: 3702), X-KKRPQDAT*S-C(Sx)-VFAMF-Z (SEQ ID NO: 3703), X-KKRPQEAT*S-C(Sx)-VFAMF-Z (SEQ ID NO: 3704), X-KKRPQFAT*S-C(Sx)-VFAMF-Z (SEQ ID NO: 3705), X-KKRPQGAT*S-C(Sx)-VFAMF-Z (SEQ ID NO: 3706), X-KKRPQHAT*S-C(Sx)-VFAMF-Z (SEQ ID NO: 3707), X-KKRPQIAT*S-C(Sx)-VFAMF-Z (SEQ ID NO: 3708), X-KKRPQKAT*S-C(Sx)-VFAMF-Z (SEQ ID NO: 3709), X-KKRPQLAT*S-C(Sx)-VFAMF-Z (SEQ ID NO: 3710), X-KKRPQNAT*S-C(Sx)-VFAMF-Z (SEQ ID NO: 3711), X-KKRPQPAT*S-C(Sx)-VFAMF-Z (SEQ ID NO: 3712), X-KKRPQQAT*S-C(Sx)-VFAMF-Z (SEQ ID NO: 3713), X-KKRPQR[Nle]T*S-C(Sx)-VFAMF-Z (SEQ ID NO: 3714), X-KKRPQRAT*[Nle]-C(Sx)-VFAMF-Z (SEQ ID NO: 3715), X-KKRPQRAT*D-C(Sx)-VFAMF-Z (SEQ ID NO: 3716), X-KKRPQRAT*E-C(Sx)-VFAMF-Z (SEQ ID NO: 3717), X-KKRPQRAT*F-C(Sx)-VFAMF-Z (SEQ ID NO: 3718), X-KKRPQRAT*G-C(Sx)-VFAMF-Z (SEQ ID NO: 3719), X-KKRPQRAT*H-C(Sx)-VFAMF-Z (SEQ ID NO: 3720), X-KKRPQRAT*I-C(Sx)-VFAMF-Z (SEQ ID NO: 3721), X-KKRPQRAT*K-C(Sx)-VFAMF-Z (SEQ ID NO: 3722), X-KKRPQRAT*L-C(Sx)-VFAMF-Z (SEQ ID NO: 3723), X-KKRPQRAT*N-C(Sx)-VFAMF-Z (SEQ ID NO: 3724), X-KKRPQRAT*P-C(Sx)-VFAMF-Z (SEQ ID NO: 3725), X-KKRPQRAT*Q-C(Sx)-VFAMF-Z (SEQ ID NO: 3726), and X-KKRPQRAT*R-C(Sx)-VFAMF-Z (SEQ ID NO: 3727);
wherein:
X is H or acetyl;
Z is OH or NH2;
S* represents S or (phospho)S;
T* represents T or (phospho)T;
Y* represents Y or (phospho)Y;
C(Sx) represents an amino acid of formula (I): 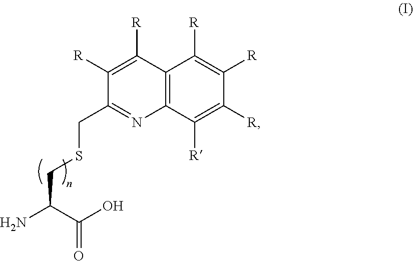
each R is independently hydrogen, or —SO₂X′, wherein at least one R group is —SO₂X′; X′ is —OR″ or —NR″R′″;
R′ is hydroxyl, amino, or thiol;
R″ is C₁₋₆ alkyl;
R′″ is hydrogen or alkyl; and
n is 1, 2 or 3.

122. The compound of claim 1, wherein the compound is X-EEPIY*V-C(Sx)-FG-Z (SEQ ID NO: 531), or X-EEPIY*V-C(Sx)-VK-Z (SEQ ID NO: 547).

123. The compound of claim 121, wherein the compound is X-C(Sx)-HS*LPRFNR-Z (SEQ ID NO: 1409).

124. The compound of claim 121, wherein the compound is X-EDPEY*I-C(Sx)-IP-Z (SEQ ID NO: 304).

125. The compound of claim 121, wherein the compound is X-KKKKVKKRPQRADS*D-C(Sx)-FA-Z (SEQ ID NO: 3457).

126. The compound of claim 121, wherein the compound is X-EEPIY*I-C(Sx)-VP-Z (SEQ ID NO: 30), or X-GAEEPIY*I-C(Sx)-VPAKKKG-Z (SEQ ID NO: 42).

127. The compound of claim 121, wherein the compound is X-C(Sx)-KT*PKKAKKL-Z (SEQ ID NO: 212), or X-C(Sx)-QS*PKKG-Z (SEQ ID NO: 207).

128. The compound of claim 121, wherein the compound is X-DANPLMA-C(Sx)-GT*LTRRHQNGRF-Z (SEQ ID NO: 1200).

129. The compound of claim 121, wherein the compound is X-C(Sx)-FS*LRRKAK-Z (SEQ ID NO: 220).

130. The compound of claim 121, wherein the compound is X-RLPWWR-C(Sx)-YT*WVRARQVNTKQR-Z (SEQ ID NO: 4513).

131. The compound of claim 121, wherein the compound is X-LKP-C(Sx)-AS*FKKFA-Z (SEQ ID NO: 5485).

132. The compound of claim 121, wherein the compound is X-KKKKVKKRPQRAHS*D-C(Sx)-FS-Z (SEQ ID NO: 3470).

133. The compound of claim 121, wherein the compound is X-SR-C(Sx)-SS*PHQ[pS]EDEEE-Z (SEQ ID NO: 4741),

134. The compound of claim 121, wherein the compound is X-KKSR-C(Sx)-DY*[Nle]T[Nle]QIG-Z (SEQ ID NO: 5051),

135. The compound of claim 121, wherein the compound is X-GDQDYLS*L-C(Sx)-VG-Z (SEQ ID NO: 651).

136. The compound of claim 121, wherein the compound is X-KKRAARAT*S-C(Sx)-VFA-Z (SEQ ID NO: 3679).

137. The compound of claim 121, wherein C(Sx) represents an amino acid of formula (II): 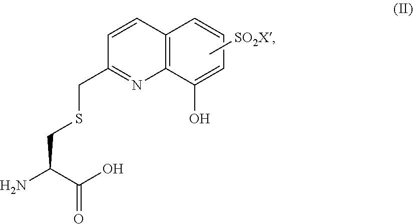
X′ is —OR″ or —NR″R′″;
R″ is C₁₋₆ alkyl; and
R′″ is hydrogen or C₁₋₆ alkyl.

138. The compound of claim 137, wherein C(Sx) represents an amino acid of formula (II): 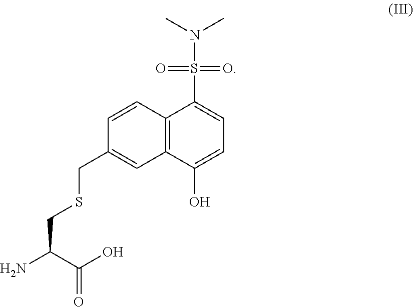 [SMILES: CN(C)S(=O)(=O)c1ccc(O)c2cc(CSC[C@H](N)C(=O)O)ccc12]
